(19) United States
(12) Patent Application Publication
(10) Pub. No.: US 2024/0199584 A1
(43) Pub. Date: Jun. 20, 2024

(54) KRAS INHIBITORS

(71) Applicant: Bristol-Myers Squibb Company, Princeton, NJ (US)

(72) Inventors: Brian Edward Fink, Yardley, PA (US); Khehyong Ngu, Pennington, NJ (US); Pravin S. Shirude, Bangalore (IN); Manoranjan Panda, Bangalore (IN); Prasada Rao Jalagam, Bangalore (IN); Laxmi Narayan Nanda, Bangalore (IN); Vishweshwaraiah Baligar, Bangalore (IN); Balaji Seshadri, Bangalore (IN); Excel Merlin Soosairaj, Bangalore (IN); Maximilian David Palkowitz, Cambridge, MA (US); Amit Kumar Chattopadhyay, Bangalore (IN)

(21) Appl. No.: 18/450,847

(22) Filed: Aug. 16, 2023

Related U.S. Application Data
(60) Provisional application No. 63/498,576, filed on Apr. 27, 2023.
(60) Provisional application No. 63/493,037, filed on Mar. 30, 2023.
(60) Provisional application No. 63/482,229, filed on Jan. 30, 2023.
(60) Provisional application No. 63/476,436, filed on Dec. 21, 2022.
(60) Provisional application No. 63/384,884, filed on Nov. 23, 2022.
(60) Provisional application No. 63/378,352, filed on Oct. 4, 2022.
(60) Provisional application No. 63/371,567, filed on Aug. 16, 2022.

(51) Int. Cl. C07D 403/12; A61K 31/498; A61K 31/4985; A61P 35/00; C07D 403/14; C07D 405/14; C07D 413/14

(52) U.S. Cl. CPC C07D 403/12; A61K 31/498; A61K 31/4985; A61P 35/00; C07D 403/14; C07D 405/14; C07D 413/14

(57) ABSTRACT

The present disclosure provides KRAS inhibitors. Methods of treating cancers using the compounds are also provided.

### CROSS-REFERENCE TO RELATED APPLICATIONS

This application claims the priority benefit of U.S. Provisional Application Nos. 63/498,576, filed Apr. 27, 2023; 63/493,037, filed Mar. 30, 2023; 63/482,229, filed Jan. 30, 2023; 63/476,436, filed Dec. 21, 2022; 63/384,884, filed Nov. 23, 2022, 63/378,352, filed Oct. 4, 2022, and 63/371,567, filed Aug. 16, 2022; all of which are incorporated by reference herein in their entireties.

### FIELD

The present disclosure provides KRAS inhibitors. Methods of treating cancers using the inhibitors are also provided.

### BACKGROUND

The KRAS oncogene is a member of the RAS family of GTPases that are involved in numerous cellular signaling processes. KRAS mutations are gain-of-function mutations that are present in up to 30% of all tumors, including as many as 90% of pancreatic cancers. Single nucleotide substitutions that result in missense mutations at codons 12 and 13 of the KRAS primary amino acid sequence comprise approximately 40% of KRAS driver mutations in lung adenocarcinoma, with a G12C transversion being the most common activating mutation. KRAS G12C mutations occur in about 13% of lung adenocarcinomas and about 3% of colorectal adenocarcinomas and are also present in cancers of the breast, bladder, cervix, ovaries, pancreas and uterus. KRAS G12D mutations occur in 28% of all pancreatic ductal adenocarcinoma patients, 13% of all colorectal carcinoma patients, 4% of all non-small cell lung carcinoma patients and 3% of all gastric carcinoma patients. See, for example, https://www.mycancergenome.org/content/alteration/kras-g12d/. Due to the clinical significance of this protein, many attempts have been made to develop RAS inhibitors, but such attempts have been mostly unsuccessful. Accordingly, agents that inhibit mutant KRAS are desired.

### SUMMARY

In a first aspect, the present disclosure provides a compound of formula (I):

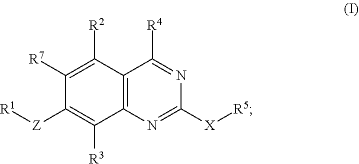

or a pharmaceutically acceptable salt thereof, wherein:
Z is a bond, O, NR^{e} or CR^{e}R^{f}, wherein R^{e} and R^{f} are independently hydrogen or C₁-C₃alkyl;

R¹ is aryl or heteroaryl, wherein the aryl and the heteroaryl are optionally substituted with one, two, three, four, or five substituents independently selected from the group consisting of C₂-C₄alkenyl, C₁-C₃alkoxy, C₁-C₃alkyl, C₂-C₄alkynyl, C₂-C₄alkynyloxy, amino, aminoC₁-C₃alkyl, cyano, cyanoC₁-C₃alkoxy, C₃-C₈cycloalkyl (e.g., C₃-C₄cycloalkyl) optionally substituted with one, two, or three halo groups, halo, haloC₁-C₃alkyl, haloC₁-C₃alkoxy, hydroxy, hydroxyC₁-C₃alkyl, heteroaryl, heterocyclyl, and phenyl, wherein the heteroaryl, heterocyclyl, and phenyl are optionally substituted with one, two, or three substituents independently selected from the group consisting of C₁-C₃alkoxy, C₁-C₃alkyl, halo, and haloC₁-C₃alkyl;
R², R³, and R⁷ are independently selected from the group consisting of hydrogen, C₁-C₃alkoxy, C₁-C₃alkyl, cyano, halo, haloC₁-C₃alkyl, —C(O)NH₂, —C(O)NH(C₁-C₃alkyl), —C(O)N(C₁-C₃alkyl)₂, and hydroxy;
R⁴ is —NHR⁵⁰ or

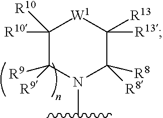

wherein
R⁵⁰ is a five membered ring optionally containing one or two heteroatoms independently selected from the group consisting of nitrogen and oxygen, wherein the ring is optionally substituted with one or two groups independently selected from C₁-C₃alkyl and oxo;
n′ is 0, 1, 2, or 3;
R⁸, R^{8′}, R⁹, R^{9′}, R¹⁰, R^{10′}, R¹³, and R^{13′} are each independently selected from the group consisting of hydrogen, C₁-C₃alkoxy, C₁-C₆alkoxyC₁-C₆alkyl, C₁-C₃alkyl, C₃-C₆cycloalkyl, cyano, halo, haloC₁-C₃alkyl, hydroxy, and hydroxyC₁-C₃alkyl; or
R⁸ and R⁹, together form a C₁-C₃alkylene; or
R⁸ and R¹⁰ together form C₁-C₃alkylene; or
R⁸ and R¹³ together form C₁-C₃alkylene; or
R⁹ and R¹³ together form C₁-C₃alkylene; or
R¹⁰ and R¹³ together form C₁-C₃alkylene; or
R⁸ and R^{8′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R⁹ and R^{9′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R¹⁰ and R^{10′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R¹³ and R^{13′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy;

W¹ is CR¹¹R¹², NR¹⁷, NR^{15″}C(O), C(O)NR^{14″}, N(C(O)(CH₂)ₙOR¹⁵), O, CH₂O, OCH₂, SO₂, SO₂NR^{15′}, or P(O)CH₃; wherein
n is 0 or 1;
R¹¹ and R¹² are independently selected from the group consisting of hydrogen, C₁-C₃alkoxy, C₁-C₆alkoxyC₁-C₆alkyl, C₁-C₃alkyl, cyano, dimethylphosphino; dimethylsulfonamide, halo, hydroxy, and methylsulfonyl; or
R¹¹ and R¹², together with the atoms to which they are attached, form a four- to six-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R¹¹ and R¹³, together with the atoms to which they are attached, form a three-, four- or five-membered ring optionally containing one or two heteroatoms independently selected from nitrogen and oxygen, wherein the ring optionally contains one or two double bonds, and wherein the ring is optionally substituted with two groups selected from the group consisting of C₁-C₃alkyl, halo, haloC₁-C₃alkyl, and hydroxy; or

R¹³ and R¹⁵, together form CH₂;
R^{15′} is C₁-C₃alkyl or C₁-C₆alkoxyC₁-C₆alkyl;
R^{15″} is hydrogen or C₁-C₃alkyl;
R¹⁷ is selected from the group consisting of C₁-C₃alkylcarbonyl, C₃-C₆cycloalkylcarbonyl, haloC₁-C₃alkylcarbonyl, methylsulfonyl, and tetrahydropyranylcarbonyl, wherein the C₃-C₆cycloalkyl and the tetrahydropyranyl are optionally substituted with one or two substituents independently selected from the group consisting of cyano, halo, and hydroxy;
X is O or NR¹⁶, wherein R¹⁶ is hydrogen or C₁-C₃alkyl;
R⁵ is selected from the group consisting of hydrogen, C₁-C₆alkoxyC₁-C₆alkyl, C₁-C₆alkyl, aryl, arylC₁-C₆alkyl, carboxyC₁-C₆alkyl, C₃-C₆cycloalkyl, C₃-C₆cycloalkylC₁-C₆alkyl, di(C₁-C₃alkyl)aminoC₂-C₆alkyl, haloC₁-C₆alkyl, heteroaryl, heteroarylC₁-C₆alkyl, heterocyclyl, heterocyclylC₁-C₆alkyl, hydroxyC₁-C₆alkyl, NR^{a}R^{b}—C(O)—C₁-C₆alkyl), NR^{a}R^{b}C₁-C₆alkyl, wherein the aryl, the aryl part of the arylC₁-C₆alkyl, the C₃-C₆cycloalkyl, the cycloalkyl part of the C₃-C₆cycloalkylC₁-C₆alkyl, the heteroaryl, the heteroaryl part of the heteroarylC₁-C₆alkyl, the heterocyclyl, the heterocyclyl part of the heterocyclylC₁-C₆alkyl, are optionally substituted with one, two, three, or four groups independently selected from C₁-C₃alkoxy, C₁-C₃alkyl, deuterated C₁-C₃alkyl, C₃-C₆cycloalkyl, (C₁-C₆alkyl)amino, (C₁-C₆alkyl)aminoC₁-C₃alkyl, amino, aminoC₁-C₃alkyl, carboxy, cyano, di(C₁-C₆alkyl)amino, di(C₁-C₆alkyl)aminoC₁-C₃alkyl, halo, haloC₁-C₃alkoxy, haloC₁-C₃alkyl, heterocyclyl, heterocyclylC₁-C₃alkyl, hydroxy, hydroxyC₁-C₃alkyl, nitro, and oxo; wherein the heterocyclyl and the heterocyclyl part of the heterocyclylC₁-C₃alkyl is further optionally substituted with one, two, or three groups independently selected from the group consisting of C₁-C₃alkoxy, C₁-C₃alkyl, halo, and haloC₁-C₃alkyl; or
R⁵ and R¹⁶, together with the nitrogen atom to which they are attached, form a heterocyclic group optionally substituted with one, two, three, four, or five groups independently selected from the group consisting of one, two, three, or four groups independently selected from the group consisting of C₁-C₃alkoxy, C₁-C₃alkoxyalkyl, C₁-C₃alkyl, amino, aminoC₁-C₃alkyl, hydroxy, and hydroxyC₁-C₃alkyl; and
one of R^{a} and R^{b} is selected from the group consisting of hydrogen and C₁-C₃alkyl and the other is selected from the group consisting of hydrogen, C₁-C₃alkyl, C₁-C₃alkoxycarbonyl, C₁-C₃alkylcarbonyl, arylC₁-C₆alkyl, C₃-C₆cycloalkyl, and C₃-C₆cycloalkylC₁-C₆alkyl.

In certain aspects, R⁷ is chloro.

In some aspects, R⁷ is hydrogen.

In certain aspects, the present disclosure provides a compound of formula (II):

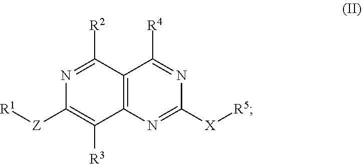

or a pharmaceutically acceptable salt thereof, wherein:
Z is a bond, O, NR^{e} or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from the group consisting of C₂-C₄alkenyl, C₁-C₃alkoxy, C₁-C₃alkyl, C₂-C₄alkynyl, C₂-C₄alkynyloxy, amino, aminoC₁-C₃alkyl, cyano, cyanoC₁-C₃alkoxy, C₃-C₈cycloalkyl (for example, C₃-C₄ cycloalkyl) optionally substituted with one, two, or three halo groups, halo, haloC₁-C₃alkyl, haloC₁-C₃alkoxy, hydroxy, hydroxyC₁-C₃alkyl, heteroaryl, heterocyclyl, and phenyl, wherein the heteroaryl, heterocyclyl, and phenyl are optionally substituted with one, two, or three substituents independently selected from the group consisting of C₁-C₃alkoxy, C₁-C₃alkyl, halo, and haloC₁-C₃alkyl;

R² and R³ are independently selected from the group consisting of hydrogen, C₁-C₃alkoxy, C₁-C₃alkyl, cyano, halo, haloC₁-C₃alkyl, —C(O)NH₂, —C(O)NH(C₁-C₃alkyl), —C(O)N(C₁-C₃alkyl)₂, and hydroxy;
R⁴ is —NHR⁵⁰ or

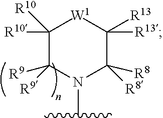

wherein
R⁵⁰ is a five membered ring optionally containing one or two heteroatoms independently selected from the group consisting of nitrogen and oxygen, wherein the ring is optionally substituted with one or two groups independently selected from C₁-C₃alkyl and oxo;

n′ is 0, 1, 2, 3;

R⁸, R^{8′}, R⁹, R^{9′}, R¹⁰, R^{10′}, R¹³, and R^{13′} are each independently selected from the group consisting of hydrogen, C₁-C₃alkoxy, C₁-C₆alkoxyC₁-C₆alkyl, C₁-C₃alkyl, C₃-C₆cycloalkyl, cyano, halo, haloC₁-C₃alkyl, hydroxy, and hydroxyC₁-C₃alkyl; or
R⁸ and R⁹, together form a C₁-C₃alkylene; or
R⁸ and R¹⁰ together form C₁-C₃alkylene; or
R⁸ and R¹³ together form C₁-C₃alkylene; or
R⁹ and R¹³ together form C₁-C₃alkylene; or
R¹⁰ and R¹³ together form C₁-C₃alkylene; or
R⁸ and R_{8′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R⁹ and R^{9′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R¹⁰ and R^{10′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R¹³ and R^{13′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy;
W¹ is CR¹¹R¹², NR¹⁷, NR^{15″}C(O), C(O)NR^{15″}, N(C(O)(CH₂)ₙOR¹⁵), O, CH₂O, OCH₂, SO₂, SO₂NR^{15′}, or P(O)CH₃; wherein
n is 0 or 1;
R¹¹ and R¹² are independently selected from the group consisting of hydrogen, C₁-C₃alkoxy, C₁-C₆alkoxyC₁-C₆alkyl, C₁-C₃alkyl, cyano, dimethylphosphino; dimethylsulfonamide, halo, hydroxy, and methylsulfonyl; or

R¹¹ and R¹², together with the atoms to which they are attached, form a four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or

R¹¹ and R¹³, together with the atoms to which they are attached, form a three-, four- or five-membered ring optionally containing one or two heteroatoms independently selected from nitrogen and oxygen, wherein the ring optionally contains one or two double bonds, and wherein the ring is optionally substituted with two groups selected from the group consisting of C₁-C₃alkyl, halo, haloC₁-C₃alkyl, and hydroxy; or

R¹³ and R¹⁵, together form CH₂;
R^{15′} is C₁-C₃alkyl or C₁-C₆alkoxyC₁-C₆alkyl;
R^{15″} is hydrogen or C₁-C₃alkyl;
R¹⁷ is selected from the group consisting of C₁-C₃alkylcarbonyl, C₃-C₆cycloalkylcarbonyl, haloC₁-C₃alkylcarbonyl, methylsulfonyl, and tetrahydropyranylcarbonyl, wherein the C₃-C₆cycloalkyl and the tetrahydropyranyl are optionally substituted with one or two substituents independently selected from the group consisting of cyano, halo, and hydroxy;
X is O or NR¹⁶, wherein R¹⁶ is hydrogen or C₁-C₃alkyl;
R⁵ is selected from the group consisting of hydrogen, C₁-C₆alkoxyC₁-C₆alkyl, C₁-C₆alkyl, aryl, arylC₁-C₆alkyl, carboxyC₁-C₆alkyl, C₃-C₆cycloalkyl, C₃-C₆cycloalkylC₁-C₆alkyl, di(C₁-C₃alkyl)aminoC₂-C₆alkyl, haloC₁-C₆alkyl, heteroaryl, heteroarylC₁-C₆alkyl, heterocyclyl, heterocyclylC₁-C₆alkyl, hydroxyC₁-C₆alkyl, NR^{a}R^{b}—C(O)—C₁-C₆alkyl), NR^{a}R^{b}C₁-C₆alkyl, wherein the aryl, the aryl part of the arylC₁-C₆alkyl, the C₃-C₆cycloalkyl, the cycloalkyl part of the C₃-C₆cycloalkylC₁-C₆alkyl, the heteroaryl, the heteroaryl part of the heteroarylC₁-C₆alkyl, the heterocyclyl, the heterocyclyl part of the heterocyclylC₁-C₆alkyl, are optionally substituted with one, two, three, or four groups independently selected from the group consisting of C₁-C₃alkoxy, C₁-C₃alkyl, deuterated C₁-C₃alkyl, C₃-C₆cycloalkyl, (C₁-C₆alkyl)amino, (C₁-C₆alkyl)aminoC₁-C₃alkyl, amino, aminoC₁-C₃alkyl, carboxy, cyano, di(C₁-C₆alkyl)amino, di(C₁-C₆alkyl)aminoC₁-C₃alkyl, halo, haloC₁-C₃alkoxy, haloC₁-C₃alkyl, heterocyclyl, heterocyclylC₁-C₃alkyl, hydroxy, hydroxyC₁-C₃alkyl, nitro, and oxo; wherein the heterocyclyl and the heterocyclyl part of the heterocyclylC₁-C₃alkyl is further optionally substituted with one, two, or three groups independently selected from the group consisting of C₁-C₃alkoxy, C₁-C₃alkyl, halo, and haloC₁-C₃alkyl; or
R⁵ and R¹⁶, together with the nitrogen atom to which they are attached, form a heterocyclic group optionally substituted with one, two, three, four, or five groups independently selected from the group consisting of one, two, three, or four groups independently selected from the group consisting of C₁-C₃alkoxy, C₁-C₃alkoxyalkyl, C₁-C₃alkyl, amino, aminoC₁-C₃alkyl, hydroxy, and hydroxyC₁-C₃alkyl; and
one of R^{a} and R^{b} is selected from hydrogen and C₁-C₃alkyl and the other is selected from the group consisting of hydrogen, C₁-C₃alkyl, C₁-C₃alkoxycarbonyl, C₁-C₃alkylcarbonyl, arylC₁-C₆alkyl, C₃-C₆cycloalkyl, and C₃-C₆cycloalkylC₁-C₆alkyl.

In some aspects, the present disclosure provides a compound of formula (II) wherein R² is methoxy.

In some aspects, the present disclosure provides a compound of formula (I) or (II), or a pharmaceutically acceptable salt thereof, wherein R⁴ is —NHR⁵⁰, and R⁵⁰ is a five membered ring optionally containing one or two heteroatoms independently selected from the group consisting of nitrogen and oxygen, wherein the ring is optionally substituted with one or two groups independently selected from C₁-C₃alkyl and oxo.

In some aspects, the present disclosure provides a compound of formula (I) or (II), or a pharmaceutically acceptable salt thereof, wherein W¹ is CR¹¹R¹², N(C(O)(CH₂)ₙOR¹⁵), O, SO₂, SO₂NR^{15′}, or P(O)CH₃, wherein R¹¹, R¹², n, R¹⁵, and R^{15′} are as defined above.

In some aspects, the present disclosure provides a compound of formula (I) or (II), or a pharmaceutically acceptable salt thereof, wherein W¹ is CR¹¹R¹².

In some aspects, the present disclosure provides a compound of formula (I) or (II), or a pharmaceutically acceptable salt thereof, wherein W¹ is NR¹⁷.

In some aspects, the present disclosure provides a compound of formula (I) or (II), or a pharmaceutically acceptable salt thereof, wherein W¹ is NR^{15″}C(O).

In some aspects, the present disclosure provides a compound of formula (I) or (II), or a pharmaceutically acceptable salt thereof, wherein W¹ is C(O)NR^{15″}.

In some aspects, the present disclosure provides a compound of formula (I) or (II), or a pharmaceutically acceptable salt thereof, wherein W¹ is N(C(O)(CH₂)ₙOR¹⁵).

In some aspects, the present disclosure provides a compound of formula (I) or (II), or a pharmaceutically acceptable salt thereof, wherein W¹ is O.

In some aspects, the present disclosure provides a compound of formula (I) or (II), or a pharmaceutically acceptable salt thereof, wherein W¹ is CH₂O.

In some aspects, the present disclosure provides a compound of formula (I) or (II), or a pharmaceutically acceptable salt thereof, wherein W¹ is OCH₂.

In some aspects, the present disclosure provides a compound of formula (I) or (II), or a pharmaceutically acceptable salt thereof, wherein W¹ is SO₂.

In some aspects, the present disclosure provides a compound of formula (I) or (II), or a pharmaceutically acceptable salt thereof, wherein W¹ is SO₂NR^{15′}.

In some aspects, the present disclosure provides a compound of formula (I) or (II), or a pharmaceutically acceptable salt thereof, wherein W¹ is P(O)CH₃.

In some aspects, the present disclosure provides a compound of formula (I) or (II), or a pharmaceutically acceptable salt thereof, wherein R⁴ is

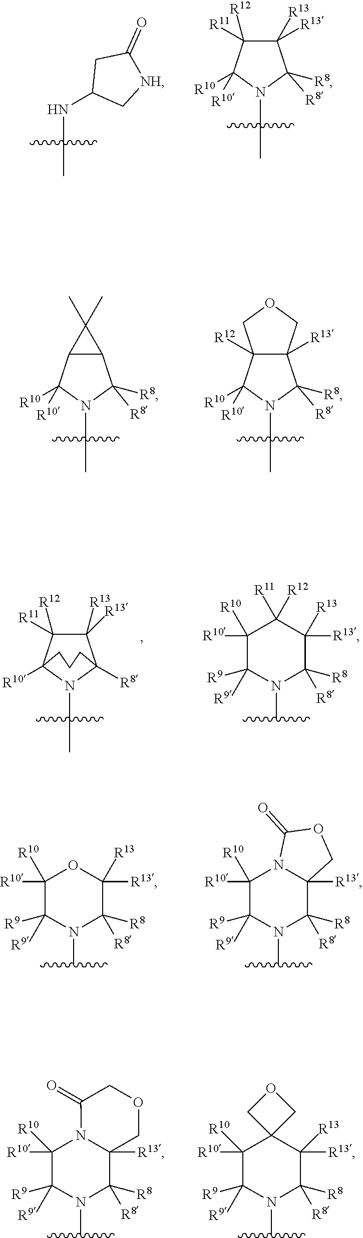

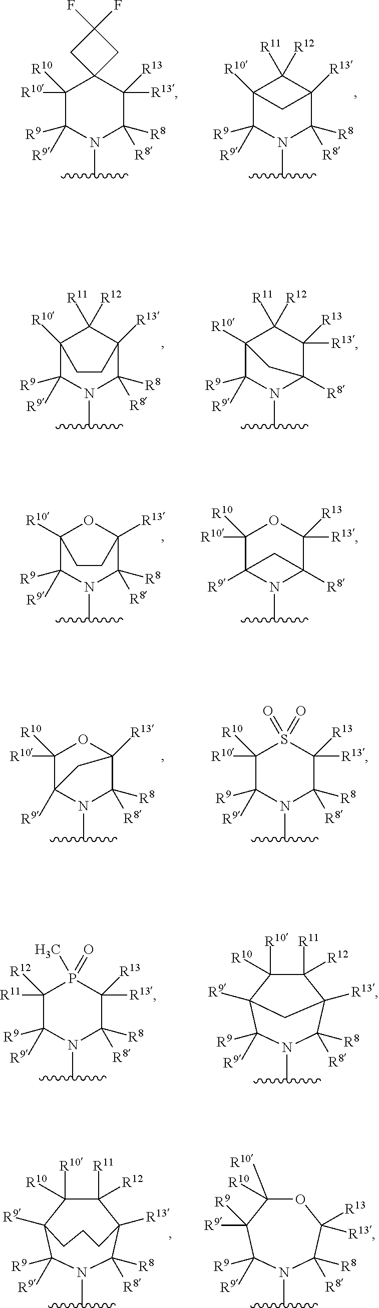

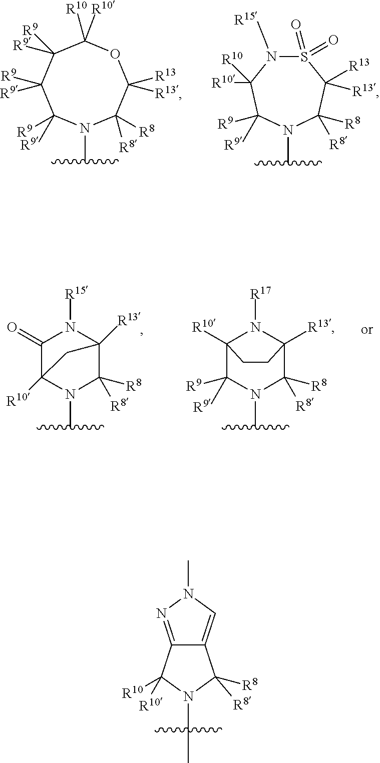

wherein [symbol] represents the point of attachment to the core of formula (I) or (II).

In some aspects, the present disclosure provides a compound of formula (I) or (II), or a pharmaceutically acceptable salt thereof, wherein R⁴ is

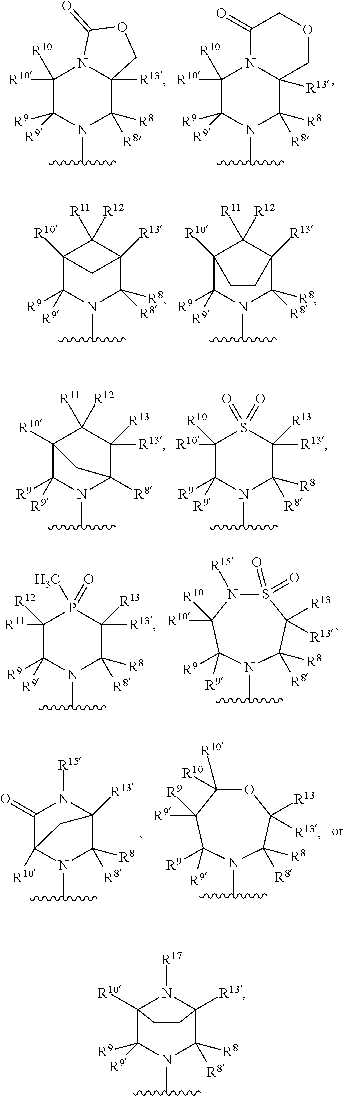

wherein [symbol] represents the point of attachment to the core of formula (II).

In some aspects, the present disclosure provides a compound of formula (I) or (II), or a pharmaceutically acceptable salt thereof, wherein R² is hydrogen.

In some aspects, the present disclosure provides a compound of formula (I) or (II), or a pharmaceutically acceptable salt thereof, wherein R³ is halo.

In some aspects, the present disclosure provides a compound of formula (I) or (II), or a pharmaceutically acceptable salt thereof, wherein X is O.

In some aspects, the present disclosure provides a compound of formula (I) or (II), or a pharmaceutically acceptable salt thereof, wherein R⁵ is selected from the group consisting of:

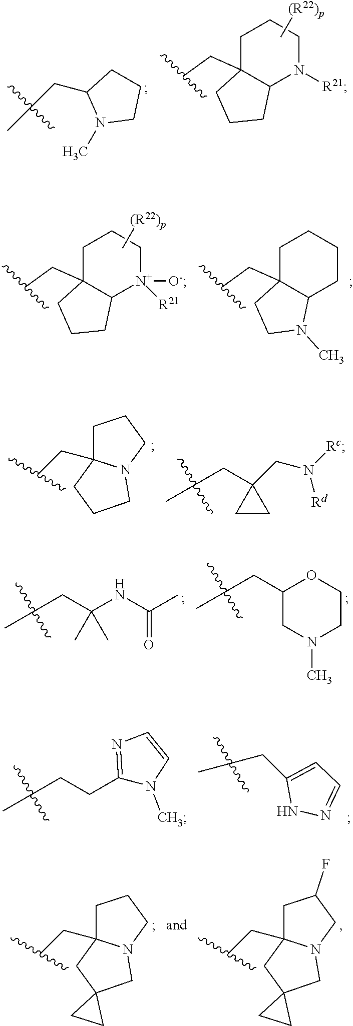

wherein each ring is optionally substituted with 1, 2, or 3 groups independently selected from the group consisting of C₁-C₃alkoxy, C₁-C₃alkoxyC₁-C₃alkyl, C₁-C₃alkyl, deuterated C₁-C₃alkyl, C₃-C₆cycloalkyl, benzyl, halo, haloC₁-C₃alkyl, hydroxy, hydroxyC₁-C₃alkyl, and oxo; and wherein
R^{c} and R^{d}, together with the nitrogen atom to which they are attached, form a five- to ten-membered ring monocyclic or bicyclic ring optionally containing one additional heteroatom selected from nitrogen, oxygen, and sulfur, wherein the ring is optionally substituted with one, two, or three groups independently selected from C₁-C₃alkoxy, C₁-C₃alkoxyC₁-C₃alkyl, C₁-C₃alkyl, benzyl, halo, haloC₁-C₃alkyl, hydroxy, hydroxyC₁-C₃alkyl, and oxo; or
one of R^{c} and R^{d} is selected from hydrogen and C₁-C₃alkyl and the other is selected from hydrogen, C₁-C₃alkyl, C₁-C₃alkoxycarbonyl, and C₁-C₃alkylcarbonyl.

In some aspects, the present disclosure provides a compound of formula (I) or (II), or a pharmaceutically acceptable salt thereof, wherein R⁵ is —(C₁-C₃alkyl)-R⁶, wherein R⁶ is a three to six-membered monocyclic ring system, an eight- or nine-membered bicyclic fused saturated ring system, a ten-membered tricyclic saturated ring system, or a twelve-membered tetracyclic saturated ring system, wherein each ring system optionally contains one or more nitrogen, oxygen and/or sulfur atoms, and wherein each ring system is optionally substituted with one to four groups independently selected from the group consisting of C₁-C₃alkyl, halo, oxo, and (4- to 6-membered heterocyclyl)C₁-C₃alkyl; wherein the heterocyclyl part of the (4- to 6-membered heterocyclyl)C₁-C₃alkyl is further optionally substituted with a halo group.

In some aspects, the present disclosure provides a compound of formula (I) or (II), or a pharmaceutically acceptable salt thereof, wherein R⁵ is

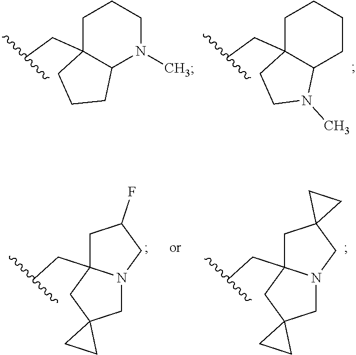 [SMILES: *CC12CCCC1N(C)CCC2 ; *CC12CCCCC1N(C)CC2 ; ";" ; *CC12CC(F)CN1CC1(CC1)C2 ; *CC12CC3(CC3)CN1CC1(CC1)C2]

and [symbol] represents the point of attachment to X.

In some aspects, the present disclosure provides a compound of formula (I) or (II), or a pharmaceutically acceptable salt thereof, salt thereof, wherein R⁵ is

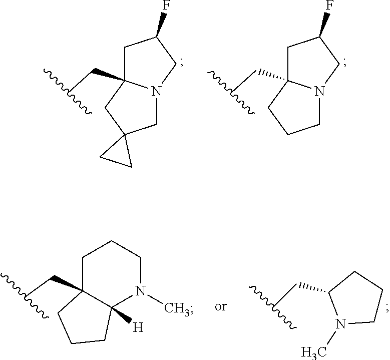

[symbol]

represents the point of attachment to X.

In some aspects, the present disclosure provides a compound of formula (I) or (II), or a pharmaceutically acceptable salt thereof, wherein R⁵ is

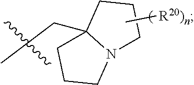

wherein n is 0, 1, or 2;
each R²⁰ is halo; and
[symbol] represents the point of attachment to X.

In some aspects, the present disclosure provides a compound of formula (I) or (II), or a pharmaceutically acceptable salt thereof, wherein R⁵ is

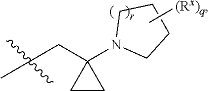

wherein r is 1 or 2; q is 0, 1, or 2; R^{x} is selected from the group consisting of C₁-C₃alkoxy, C₁-C₃alkoxyC₁-C₃alkyl, C₁-C₃alkyl, benzyl, halo, haloC₁-C₃alkyl, hydroxy, hydroxyC₁-C₃alkyl, and oxo, and [symbol] denotes the point of attachment to X.

In some aspect, the present disclosure provides a compound of formula (I) or (II), or a pharmaceutically acceptable salt thereof, wherein R⁵ is

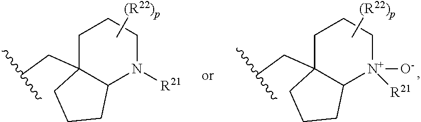

wherein R²¹ is selected from the group consisting of C₁-C₃alkyl, deuterated C₁-C₃alkyl, and C₃-C₆cycloalkyl, R²² is halo; p is 0 or 1; and [symbol] denotes the point of attachment to X.

In some aspects, the present disclosure provides a compound of formula (I) or (II), or a pharmaceutically acceptable salt thereof, wherein R⁵ is

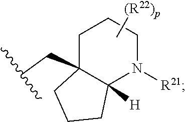

wherein
[symbol] represents the point of attachment to X;
p is 0 or 1;
R²¹ is selected from the group consisting of C₁-C₃alkyl, deuterated C₁-C₃alkyl, and C₃-C₆cycloalkyl; and
R²² is halo; p is 0 or 1; and [symbol] denotes the point of attachment to X.

In some aspects, the present disclosure provides a compound of formula (I) or (II), or a pharmaceutically acceptable salt thereof, wherein R⁵ is

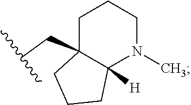 [SMILES: *C[C@]12CCC[C@H]1N(C)CCC2]

wherein
[symbol] represents the point of attachment to X.

In some aspects, the present disclosure provides a compound of formula (I) or (II), or a pharmaceutically acceptable salt thereof, wherein Z is a bond.

In some aspects, the present disclosure provides a compound of formula (I) or (II), or a pharmaceutically acceptable salt thereof, wherein R¹ is a monocyclic heteroaryl ring containing one, two, or three nitrogen atoms, wherein the ring is optionally substituted with one, two, three, four, or five substituents independently selected from the group consisting of C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkyl, haloC₁-C₃alkoxy, hydroxy, and hydroxyC₁-C₃alkyl.

In some aspects, the present disclosure provides a compound of formula (I) or (II), or a pharmaceutically acceptable salt thereof, wherein R¹ is

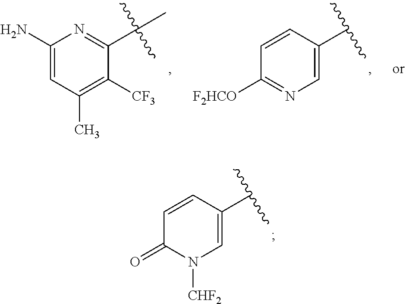

wherein [symbol] denotes the point of attachment to the core of formula (I) or (II).

In some aspects, the present disclosure provides a compound of formula (I) or (II), or a pharmaceutically acceptable salt thereof, wherein R¹ is C₆-C₁₀aryl optionally substituted with one, two, three, four, or five substituents independently selected from the group consisting of C₁-C₃alkoxy, C₁-C₃alkyl, C₂-C₄alkenyl, C₂-C₄alkynyl, amino, aminoC₁-C₃alkyl, cyano, C₃-C₅cycloalkyl (e.g., cyclopropyl, cyclobutyl, cyclopentyl, bicyclo[1.1.1]pentanyl), halo, haloC₁-C₃alkyl, hydroxy, and hydroxyC₁-C₃alkyl.

In some aspects, the present disclosure provides a compound of formula (I) or (II), or a pharmaceutically acceptable salt thereof, wherein R¹ is aryl or heteroaryl, wherein the aryl and heteroaryl are optionally substituted with one, two, three, four, or five substituents independently selected from the group consisting of C₂-C₄alkenyl, C₁-C₃alkoxy, C₁-C₃alkyl, C₂-C₄alkynyl, C₂-C₄alkynyloxy, amino, cyano, cyanoC₁-C₃alkoxy, C₃-C₄cycloalkyl optionally substituted with one or two halo groups, halo, haloC₁-C₃alkoxy, 4- to 6-membered heterocyclyl, and hydroxy.

In some aspects, the present disclosure provides a compound of formula (I) or (II), or a pharmaceutically acceptable salt thereof, wherein R¹ is naphthyl, wherein the naphthyl is substituted with one, two, or three groups independently selected from the group consisting of C₂-C₄alkynyl, halo, and hydroxy.

In some aspects, the present disclosure provides a compound of formula (I) or (II), or a pharmaceutically acceptable salt thereof wherein R¹ is

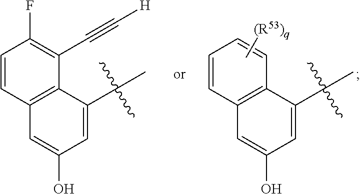

wherein R⁵³ is C₁-C₃alkyl, halo, haloC₁-C₃alkyl, haloC₁-C₃alkoxy, a 4- to 6-membered heterocyclyl, or hydroxy; q is an integer of 0 to 4; and; wherein [symbol] denotes the point of attachment to the core of formula (I) or (II).

In some aspects, the present disclosure provides a compound of formula (I) or (II), or a pharmaceutically acceptable salt thereof, wherein R¹ is phenyl, wherein the phenyl is substituted with one, two, or three groups independently selected from the group consisting of C₁-C₃alkyl, C₃-C₅cycloalkyl, heterocyclyl, and hydroxy.

In some aspects, the present disclosure provides a compound of formula (I) or (II), or a pharmaceutically acceptable salt thereof, wherein R¹ is isoquinolinyl, wherein the isoquinolinyl is substituted with one, two, or three groups independently selected from the group consisting of haloC₁-C₃alkyl and haloC₁-C₃alkoxy. In some of these aspects, R¹ is

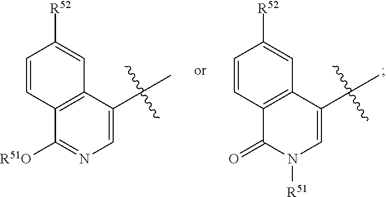

wherein R⁵¹ is haloC₁-C₃alkyl; R⁵² is hydrogen or haloC₁-C₃alkyl; and [symbol] denotes the point of attachment to the core of formula (I) or (II).

In some aspects, the present disclosure provides a compound of formula (I) or (II), or a pharmaceutically acceptable salt thereof, wherein R¹ is indolyl substituted with haloC₁-C₃alkyl. In some of these aspects, R¹ is

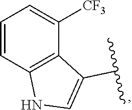

wherein [symbol] denotes the point of attachment to the core of formula (I) or (II).

In some aspects, the present disclosure provides a compound of formula (I) or (II), or a pharmaceutically acceptable salt thereof, wherein R¹ is indazolyl substituted with one, two, or three substituents selected from C₁-C₃alkyl and halo. In some of these aspects, R¹ is

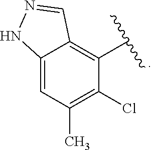 [SMILES: *c1c(Cl)c(C)cc2[nH]ncc12]

In some aspects, the present disclosure provides a compound of formula (I) or (II), or a pharmaceutically acceptable salt thereof, wherein:

R¹ is

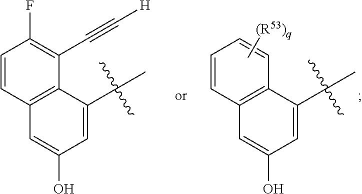

wherein R⁵³ is C₁-C₃alkyl, halo, haloC₁-C₃alkyl, haloC₁-C₃alkoxy, a 4- to 6-membered heterocyclyl, or hydroxy; and q is an integer of 0 to 4;
R⁴ is

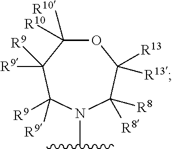

and
R⁵ is

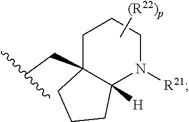

wherein
[symbol] represents the point of attachment to X;
p is 0 or 1;
R²¹ is selected from the group consisting of C₁-C₃alkyl, deuterated C₁-C₃alkyl, and C₃-C₆cycloalkyl; and
R²² is halo; p is 0 or 1;
   wherein [symbol] denotes the point of attachment to the parent molecular moiety.

In some aspects, the present disclosure provides a compound of formula (I) or (II), or a pharmaceutically acceptable salt thereof, wherein:
R¹ is

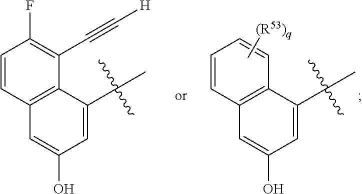

wherein R⁵³ is C₁-C₃alkyl, halo, haloC₁-C₃alkyl, haloC₁-C₃alkoxy, a 4- to 6-membered heterocyclyl, or hydroxy; q is an integer of 0 to 4;
R⁴ is

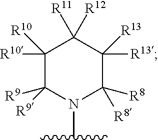

and
R⁵ is

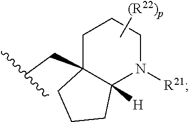

wherein
[symbol] represents the point of attachment to X;
p is 0 or 1;
R²¹ is selected from the group consisting of C₁-C₃alkyl, deuterated C₁-C₃alkyl, and C₃-C₆cycloalkyl; and
R²² is halo; p is 0 or 1;
   wherein [symbol] denotes the point of attachment to the parent molecular moiety.

In some aspects, the present disclosure provides a compound of formula (I) or (II), or a pharmaceutically acceptable salt thereof, wherein:

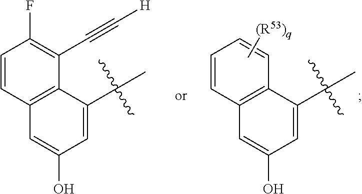

R¹ is
   wherein R⁵³ is C₁-C₃alkyl, halo, haloC₁-C₃alkyl, haloC₁-C₃alkoxy, a 4- to 6-membered heterocyclyl, or hydroxyl; q is an integer of 0 to 4;
R⁴ is

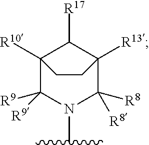

and
R⁵ is

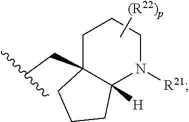

wherein
[symbol] represents the point of attachment to X;
p is 0 or 1;
R²¹ is selected from the group consisting of C₁-C₃alkyl, deuterated C₁-C₃alkyl, and C₃-C₆cycloalkyl; and
R²² is halo; p is 0 or 1;
   wherein [symbol] denotes the point of attachment to the parent molecular moiety.

In some aspects, the present disclosure provides a compound of formula (I) or (II), or a pharmaceutically acceptable salt thereof, wherein:
R¹ is

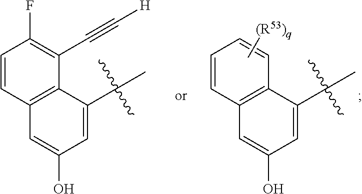

wherein R⁵³ is C₁-C₃alkyl, halo, haloC₁-C₃alkyl, haloC₁-C₃alkoxy, a 4- to 6-membered heterocyclyl, or hydroxy; and q is an integer of 0 to 4;
R⁴ is

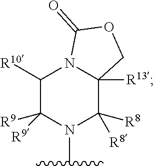

and
R⁵ is

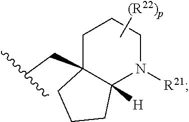

wherein
[symbol] represents the point of attachment to X;
p is 0 or 1;
R²¹ is selected from the group consisting of C₁-C₃alkyl, deuterated C₁-C₃alkyl, and C₃-C₆cycloalkyl; and
R²² is halo; p is 0 or 1;
   wherein [symbol] denotes the point of attachment to the parent molecular moiety.

In some aspects, the present disclosure provides a compound of formula (I) or (II), or a pharmaceutically acceptable salt thereof, wherein:

R¹ is

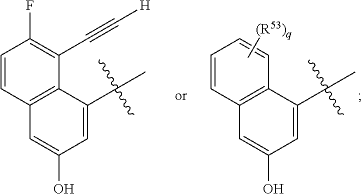

wherein R⁵³ is C₁-C₃alkyl, halo, haloC₁-C₃alkyl, haloC₁-C₃alkoxy, a 4- to 6-membered heterocyclyl, or hydroxy; and q is an integer of 0 to 4;
R⁴ is

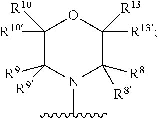

and
R⁵ is

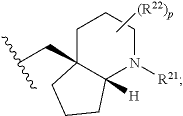

wherein
[symbol] represents the point of attachment to X;
p is 0 or 1;
R²¹ is selected from the group consisting of C₁-C₃alkyl, deuterated C₁-C₃alkyl, and C₃-C₆cycloalkyl; and
R²² is halo; p is 0 or 1;
   wherein [symbol] denotes the point of attachment to the parent molecular moiety.

In some aspects, the present disclosure provides a pharmaceutical composition comprising a compound of formula (I) or (II), or a pharmaceutically acceptable salt thereof, and one or more pharmaceutically acceptable excipients.

In some aspects, the present disclosure provides an oral dosage form comprising a compound of formula (I) or (II), or a pharmaceutically acceptable salt thereof, and one or more pharmaceutically acceptable excipients.

In some aspects of the method, the compound is an atropisomer of a compound of any of the prior aspects. In certain aspects, the compound is a stable atropisomer as described herein.

In another aspect, the present disclosure provides a method of inhibiting cell proliferation, in vitro or in vivo, the method comprising contacting a cell with a therapeutically effective amount of a compound of formula (I) or (II), or a pharmaceutically acceptable salt thereof, or a pharmaceutical composition thereof as defined herein.

In another aspect, the present disclosure provides a method for treating cancer in a subject, the method comprising administering a therapeutically effective amount of a compound or pharmaceutical composition of the present disclosure or a pharmaceutically acceptable salt thereof to a subject in need thereof.

In another aspect, the present disclosure provides a method of treating a KRAS G12D-associated disease or disorder associated with KRAS G12A, KRAS G12C, KRAS G12D, KRAS G12R, KRAS G12S, KRAS G12V, KRAS G13D and/or KRAS Q61H in a subject in need of such treatment, the method comprising administering to the subject a therapeutically effective amount of a compound of formula (I) or (II), or a pharmaceutically acceptable salt thereof, or a pharmaceutical composition thereof as defined herein.

In another aspect, the present disclosure provides a method for treating a cancer susceptible to KRAS G12A, KRAS G12C, KRAS G12D, KRAS G12R, KRAS G12S, KRAS G12V, KRAS G13D and/or KRAS Q61H inhibition in a subject in need thereof, the method comprising administering to the subject a compound of formula (I) or (II), or a pharmaceutically acceptable salt thereof.

In another aspect, the present disclosure provides a method for treating a cancer in a subject in need thereof, the method comprising administering to the subject a compound of formula (I), or a pharmaceutically acceptable salt thereof, wherein the cancer is pancreatic cancer, colorectal cancer, lung cancer, gastric cancer, breast cancer, bladder cancer, cervical cancer, ovarian cancer, cancer of the uterus or a combination thereof.

In another aspect, the present disclosure provides a compound of formula (I), or a pharmaceutically acceptable salt thereof, for use in the inhibition of inhibiting KRAS G12A, KRAS G12C, KRAS G12D, KRAS G12R, KRAS G12S, KRAS G12V, KRAS G13D and/or KRAS Q61H.

In another aspect, the present disclosure provides a compound of formula (I) or (II), or a pharmaceutically acceptable salt thereof, or a pharmaceutical composition thereof as defined herein, for use in the treatment of a disease or disorder associated with KRAS G12A, KRAS G12C, KRAS G12D, KRAS G12R, KRAS G12S, KRAS G12V, KRAS G13D and/or KRAS Q61H.

In another aspect, the present disclosure provides a use of a compound of formula (I) or (II), or a pharmaceutically acceptable salt thereof, as defined herein in the manufacture of a medicament for the treatment of cancer.

In another aspect, the present disclosure provides the use of a compound of formula (I) or (II), or a pharmaceutically acceptable salt thereof, as defined herein in the manufacture of a medicament for the inhibition of activity of inhibiting KRAS G12A, KRAS G12C, KRAS G12D, KRAS G12R, KRAS G12S, KRAS G12V, KRAS G13D and/or KRAS Q61H.

In another aspect, the present disclosure provides the use of a compound of formula (I) or (II), or a pharmaceutically acceptable salt thereof, as defined herein, in the manufacture of a medicament for the treatment of a disease or disorder associated with KRAS G12A, KRAS G12C, KRAS G12D, KRAS G12R, KRAS G12S, KRAS G12V, KRAS G13D and/or KRAS Q61H.

In another aspect, the present disclosure provides a compound selected from the group consisting of:

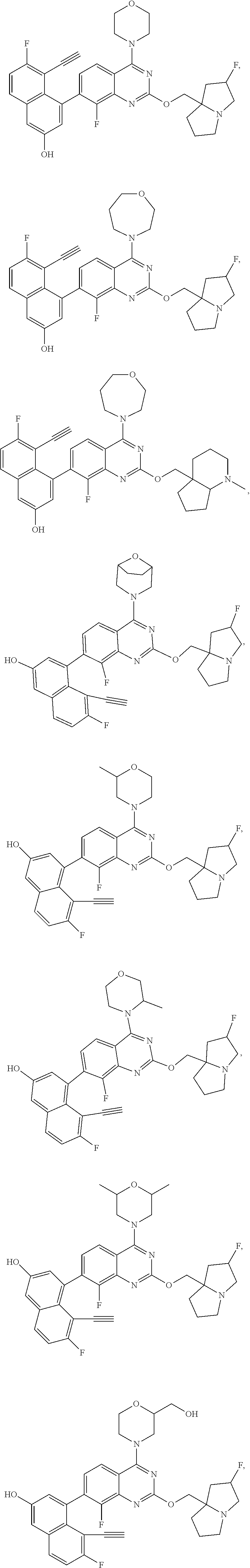

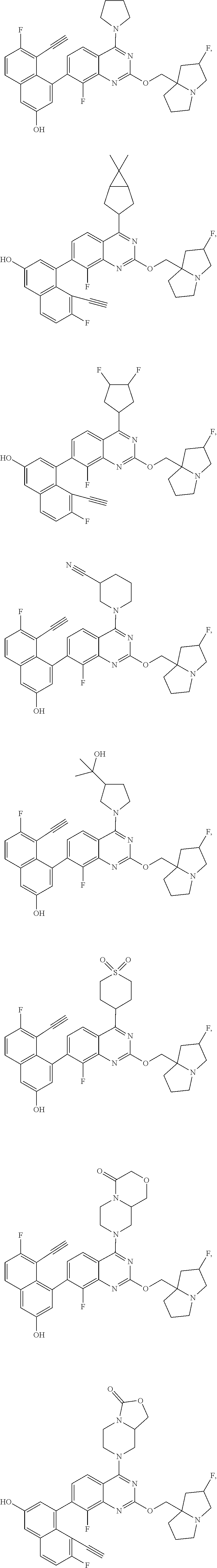

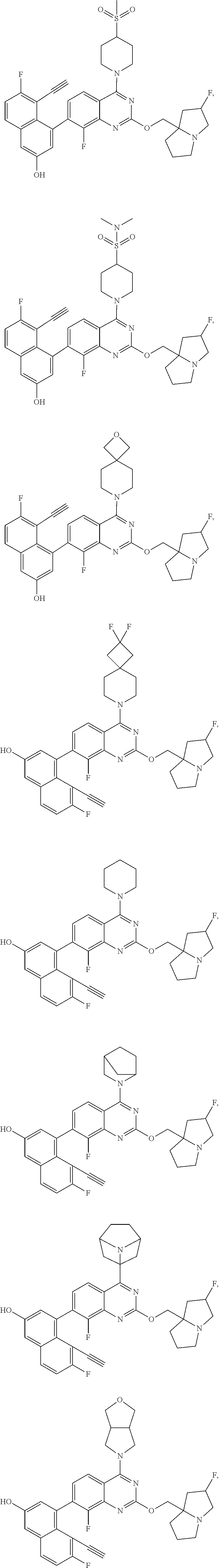

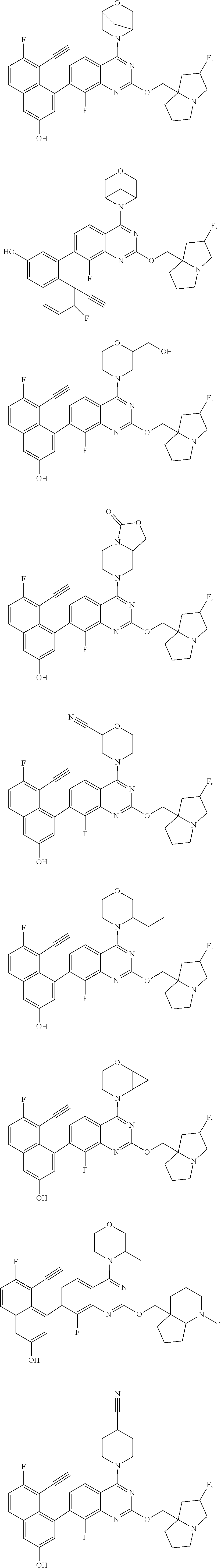

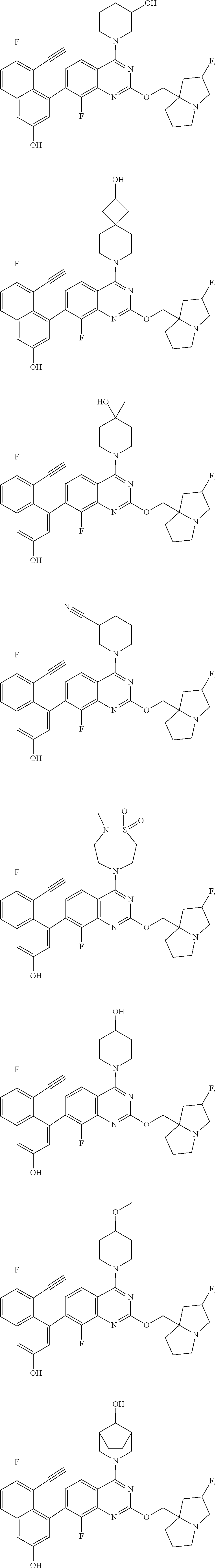

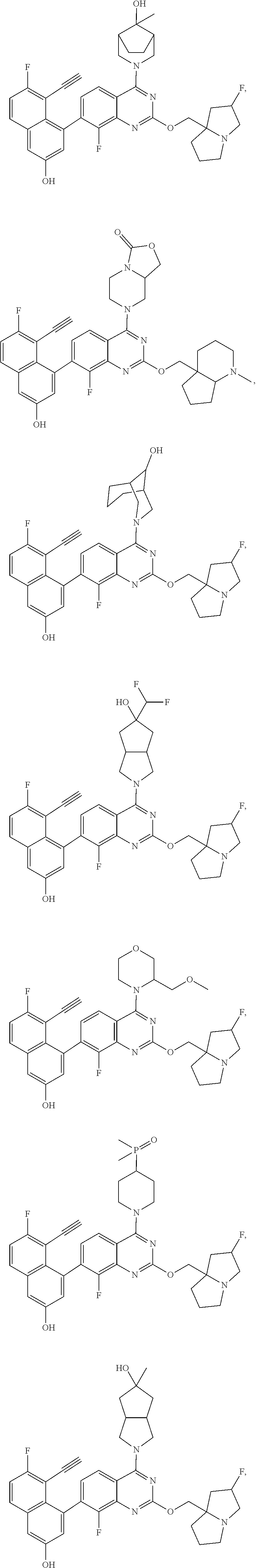

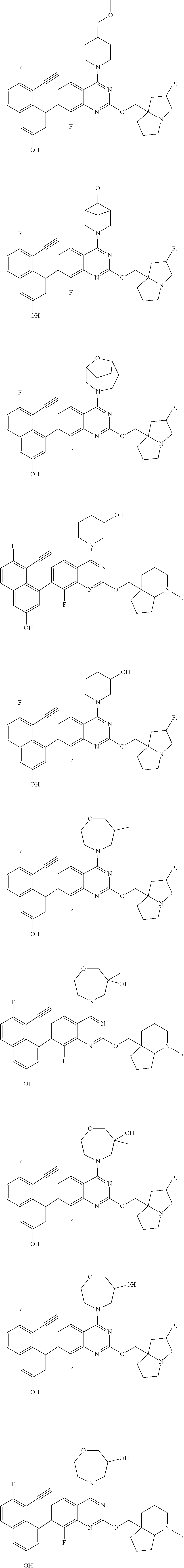

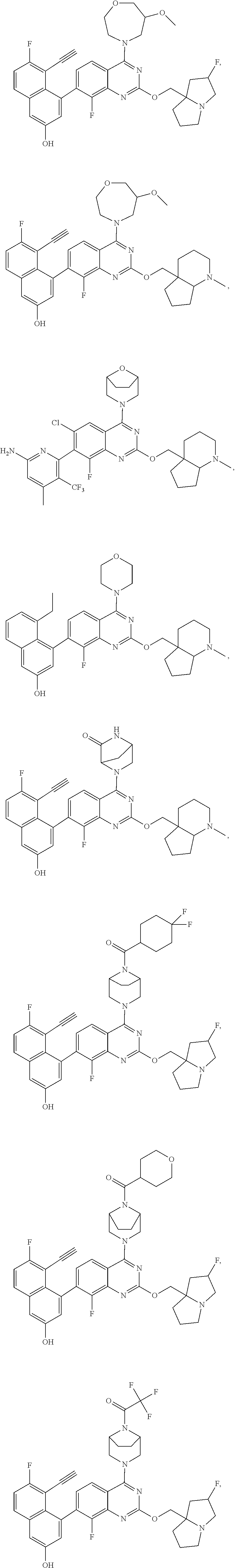

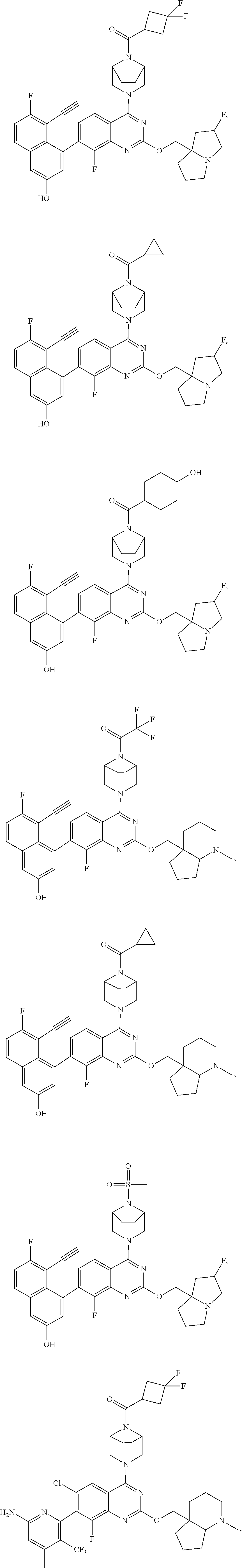

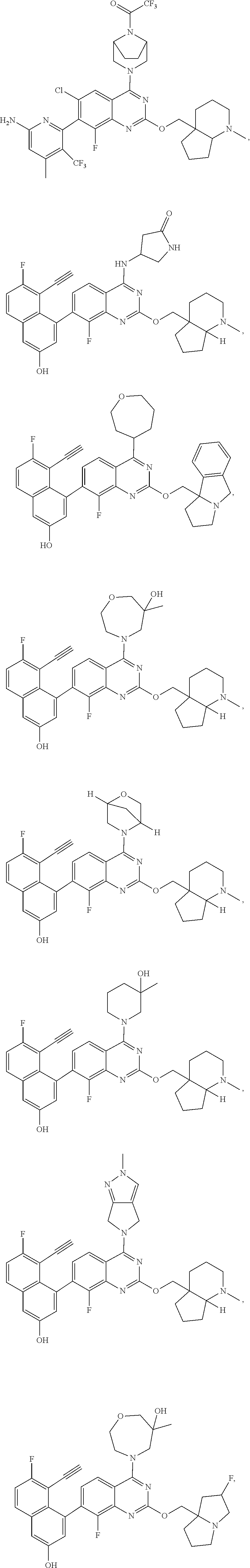

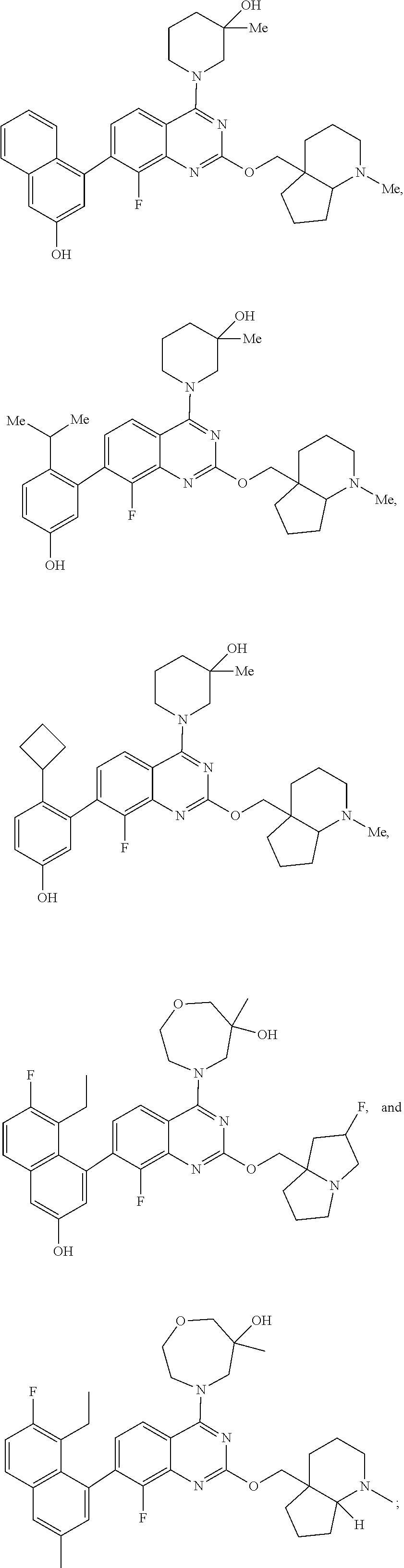 [SMILES: CN1CCCC2(COc3nc(N4CCCC(C)(O)C4)c4ccc(-c5cc(O)cc6ccccc56)c(F)c4n3)CCCC12 ; CC(C)c1ccc(O)cc1-c1ccc2c(N3CCCC(C)(O)C3)nc(OCC34CCCC3N(C)CCC4)nc2c1F ; CN1CCCC2(COc3nc(N4CCCC(C)(O)C4)c4ccc(-c5cc(O)ccc5C5CCC5)c(F)c4n3)CCCC12 ; CCc1c(F)ccc2cc(O)cc(-c3ccc4c(N5CCOCC(C)(O)C5)nc(OCC56CCCN5CC(F)C6)nc4c3F)c12 ; "and" ; CCc1c(F)ccc2cc(C)cc(-c3ccc4c(N5CCOCC(C)(O)C5)nc(OCC56CCCC5N(C)CCC6)nc4c3F)c12]

or a pharmaceutically acceptable salt thereof.

In some aspects, the present disclosure provides a compound selected from the group consisting of:

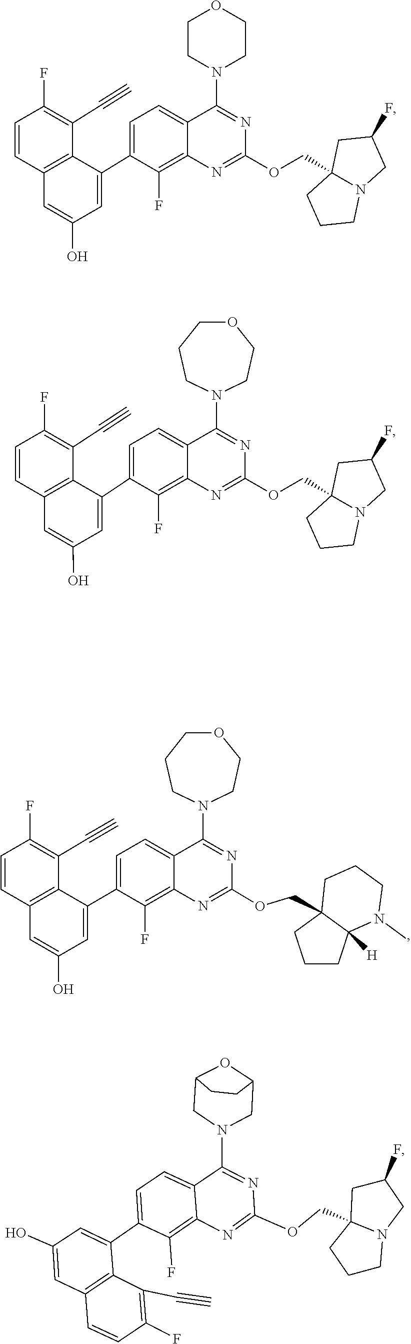 [SMILES: C#Cc1c(F)ccc2cc(O)cc(-c3ccc4c(N5CCOCC5)nc(OC[C@@]56CCCN5C[C@H](F)C6)nc4c3F)c12 ; C#Cc1c(F)ccc2cc(O)cc(-c3ccc4c(N5CCCOCC5)nc(OC[C@@]56CCCN5C[C@H](F)C6)nc4c3F)c12 ; C#Cc1c(F)ccc2cc(O)cc(-c3ccc4c(N5CCCOCC5)nc(OC[C@]56CCC[C@H]5N(C)CCC6)nc4c3F)c12 ; C#Cc1c(F)ccc2cc(O)cc(-c3ccc4c(N5CC6CCC(C5)O6)nc(OC[C@@]56CCCN5C[C@H](F)C6)nc4c3F)c12]

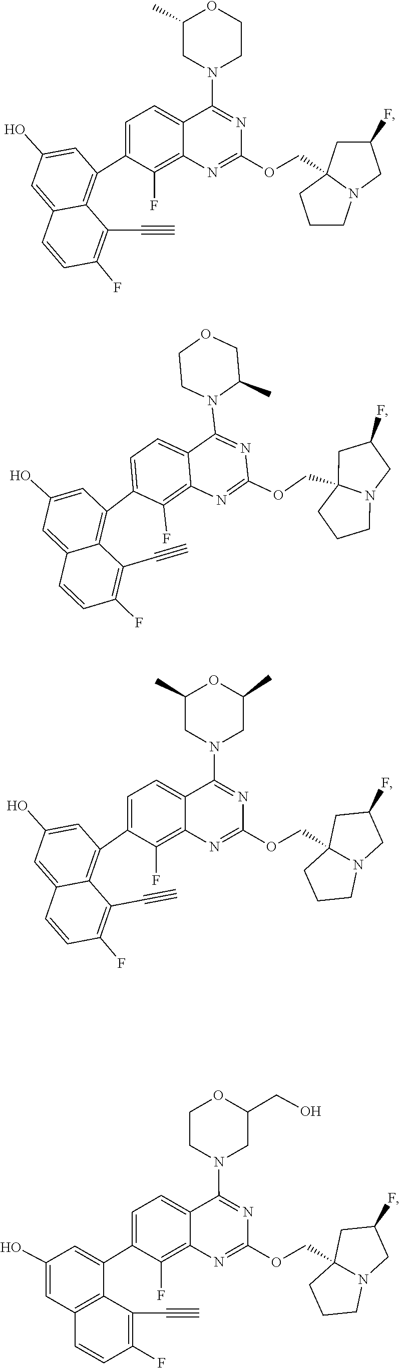 [SMILES: C#Cc1c(F)ccc2cc(O)cc(-c3ccc4c(N5CCO[C@@H](C)C5)nc(OC[C@@]56CCCN5C[C@H](F)C6)nc4c3F)c12 ; C#Cc1c(F)ccc2cc(O)cc(-c3ccc4c(N5CCOC[C@H]5C)nc(OC[C@@]56CCCN5C[C@H](F)C6)nc4c3F)c12 ; C#Cc1c(F)ccc2cc(O)cc(-c3ccc4c(N5C[C@@H](C)O[C@@H](C)C5)nc(OC[C@@]56CCCN5C[C@H](F)C6)nc4c3F)c12 ; C#Cc1c(F)ccc2cc(O)cc(-c3ccc4c(N5CCOC(CO)C5)nc(OC[C@@]56CCCN5C[C@H](F)C6)nc4c3F)c12]

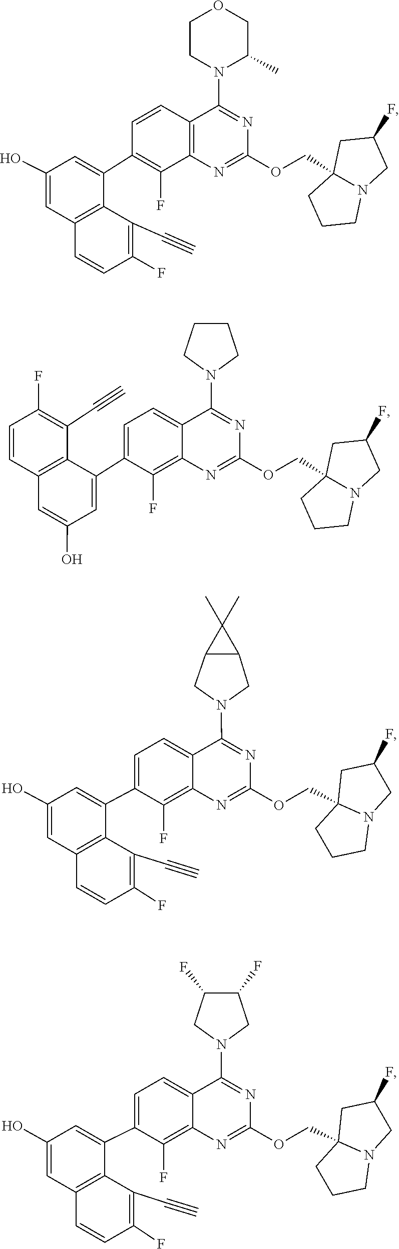 [SMILES: C#Cc1c(F)ccc2cc(O)cc(-c3ccc4c(N5CCOC[C@@H]5C)nc(OC[C@@]56CCCN5C[C@H](F)C6)nc4c3F)c12 ; C#Cc1c(F)ccc2cc(O)cc(-c3ccc4c(N5CCCC5)nc(OC[C@@]56CCCN5C[C@H](F)C6)nc4c3F)c12 ; C#Cc1c(F)ccc2cc(O)cc(-c3ccc4c(N5CC6C(C5)C6(C)C)nc(OC[C@@]56CCCN5C[C@H](F)C6)nc4c3F)c12 ; C#Cc1c(F)ccc2cc(O)cc(-c3ccc4c(N5C[C@@H](F)[C@@H](F)C5)nc(OC[C@@]56CCCN5C[C@H](F)C6)nc4c3F)c12]

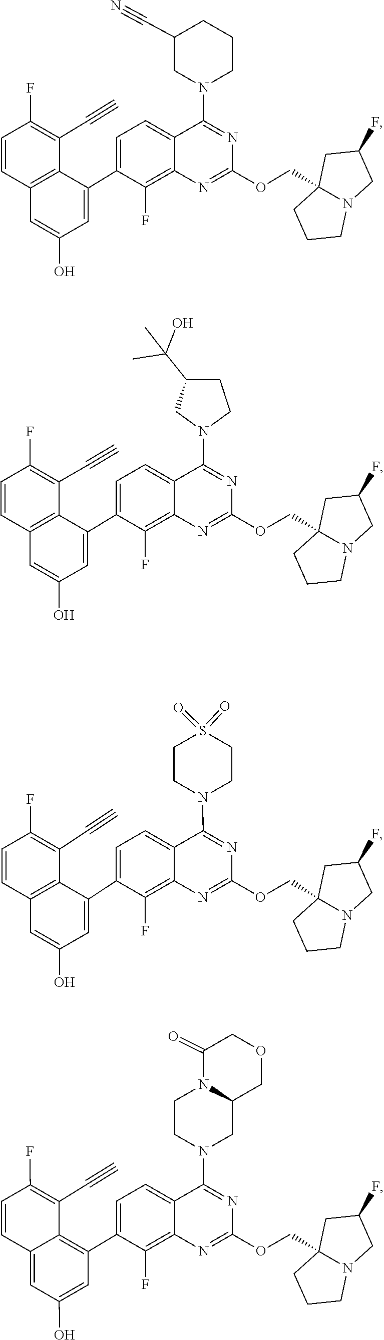 [SMILES: C#Cc1c(F)ccc2cc(O)cc(-c3ccc4c(N5CCCC(C#N)C5)nc(OC[C@@]56CCCN5C[C@H](F)C6)nc4c3F)c12 ; C#Cc1c(F)ccc2cc(O)cc(-c3ccc4c(N5CC[C@@H](C(C)(C)O)C5)nc(OC[C@@]56CCCN5C[C@H](F)C6)nc4c3F)c12 ; C#Cc1c(F)ccc2cc(O)cc(-c3ccc4c(N5CCS(=O)(=O)CC5)nc(OC[C@@]56CCCN5C[C@H](F)C6)nc4c3F)c12 ; C#Cc1c(F)ccc2cc(O)cc(-c3ccc4c(N5CCN6C(=O)COC[C@H]6C5)nc(OC[C@@]56CCCN5C[C@H](F)C6)nc4c3F)c12]

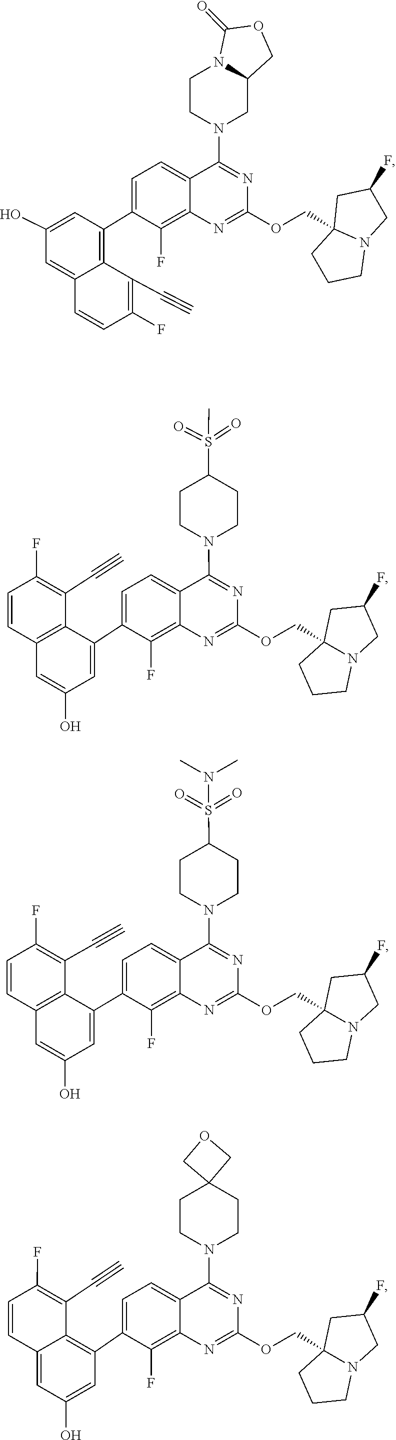 [SMILES: C#Cc1c(F)ccc2cc(O)cc(-c3ccc4c(N5CCN6C(=O)OC[C@H]6C5)nc(OC[C@@]56CCCN5C[C@H](F)C6)nc4c3F)c12 ; C#Cc1c(F)ccc2cc(O)cc(-c3ccc4c(N5CCC(S(C)(=O)=O)CC5)nc(OC[C@@]56CCCN5C[C@H](F)C6)nc4c3F)c12 ; C#Cc1c(F)ccc2cc(O)cc(-c3ccc4c(N5CCC(S(=O)(=O)N(C)C)CC5)nc(OC[C@@]56CCCN5C[C@H](F)C6)nc4c3F)c12 ; C#Cc1c(F)ccc2cc(O)cc(-c3ccc4c(N5CCC6(CC5)COC6)nc(OC[C@@]56CCCN5C[C@H](F)C6)nc4c3F)c12]

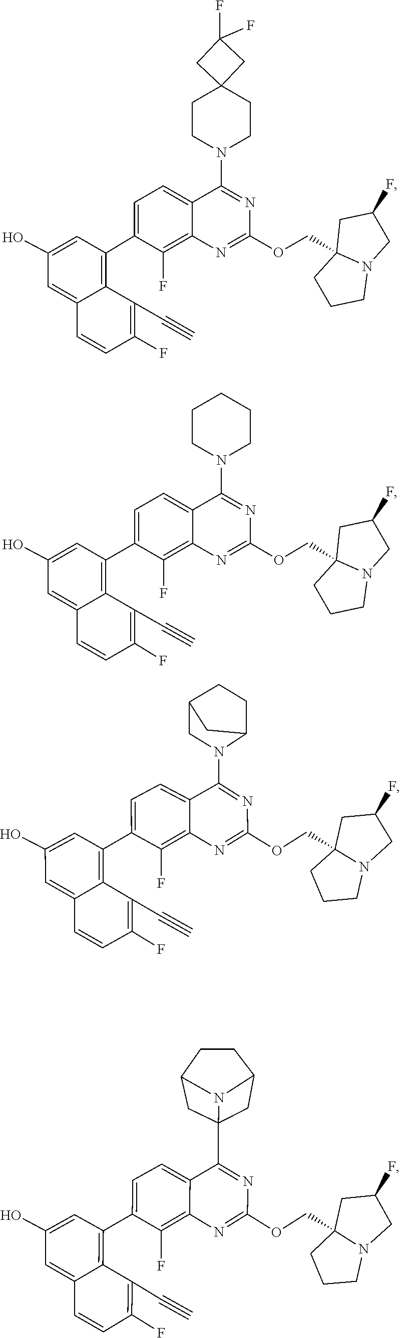 [SMILES: C#Cc1c(F)ccc2cc(O)cc(-c3ccc4c(N5CCC6(CC5)CC(F)(F)C6)nc(OC[C@@]56CCCN5C[C@H](F)C6)nc4c3F)c12 ; C#Cc1c(F)ccc2cc(O)cc(-c3ccc4c(N5CCCCC5)nc(OC[C@@]56CCCN5C[C@H](F)C6)nc4c3F)c12 ; C#Cc1c(F)ccc2cc(O)cc(-c3ccc4c(N5CC6CCC5C6)nc(OC[C@@]56CCCN5C[C@H](F)C6)nc4c3F)c12 ; C#Cc1c(F)ccc2cc(O)cc(-c3ccc4c(C56CC7CCC(C5)N76)nc(OC[C@@]56CCCN5C[C@H](F)C6)nc4c3F)c12]

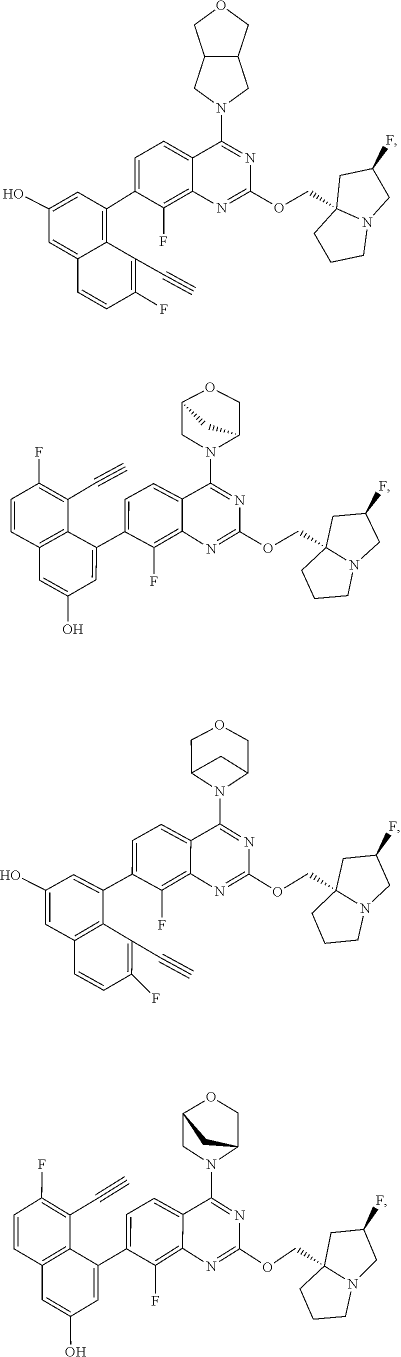 [SMILES: C#Cc1c(F)ccc2cc(O)cc(-c3ccc4c(N5CC6COCC6C5)nc(OC[C@@]56CCCN5C[C@H](F)C6)nc4c3F)c12 ; C#Cc1c(F)ccc2cc(O)cc(-c3ccc4c(N5C[C@@H]6C[C@H]5CO6)nc(OC[C@@]56CCCN5C[C@H](F)C6)nc4c3F)c12 ; C#Cc1c(F)ccc2cc(O)cc(-c3ccc4c(N5C6COCC5C6)nc(OC[C@@]56CCCN5C[C@H](F)C6)nc4c3F)c12 ; C#Cc1c(F)ccc2cc(O)cc(-c3ccc4c(N5C[C@H]6C[C@@H]5CO6)nc(OC[C@@]56CCCN5C[C@H](F)C6)nc4c3F)c12]

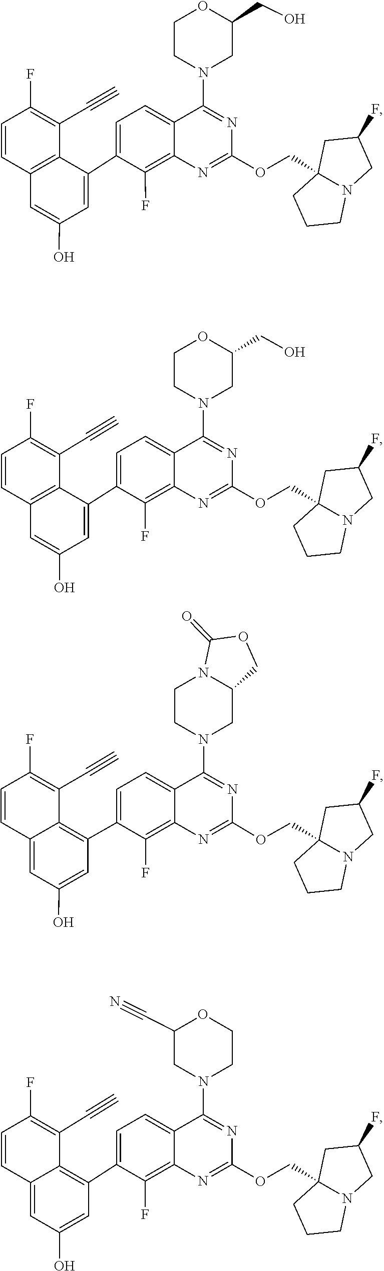 [SMILES: C#Cc1c(F)ccc2cc(O)cc(-c3ccc4c(N5CCO[C@@H](CO)C5)nc(OC[C@@]56CCCN5C[C@H](F)C6)nc4c3F)c12 ; C#Cc1c(F)ccc2cc(O)cc(-c3ccc4c(N5CCO[C@H](CO)C5)nc(OC[C@@]56CCCN5C[C@H](F)C6)nc4c3F)c12 ; C#Cc1c(F)ccc2cc(O)cc(-c3ccc4c(N5CCN6C(=O)OC[C@@H]6C5)nc(OC[C@@]56CCCN5C[C@H](F)C6)nc4c3F)c12 ; C#Cc1c(F)ccc2cc(O)cc(-c3ccc4c(N5CCOC(C#N)C5)nc(OC[C@@]56CCCN5C[C@H](F)C6)nc4c3F)c12]

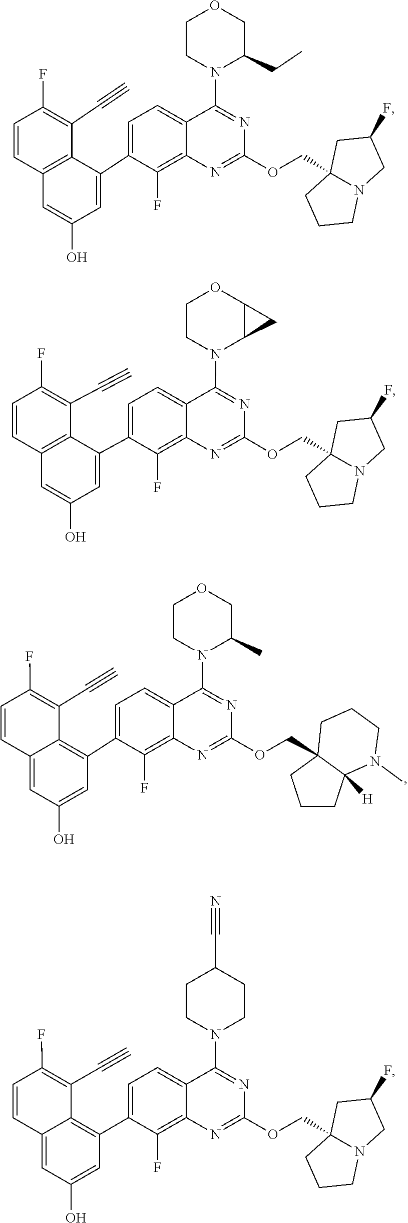

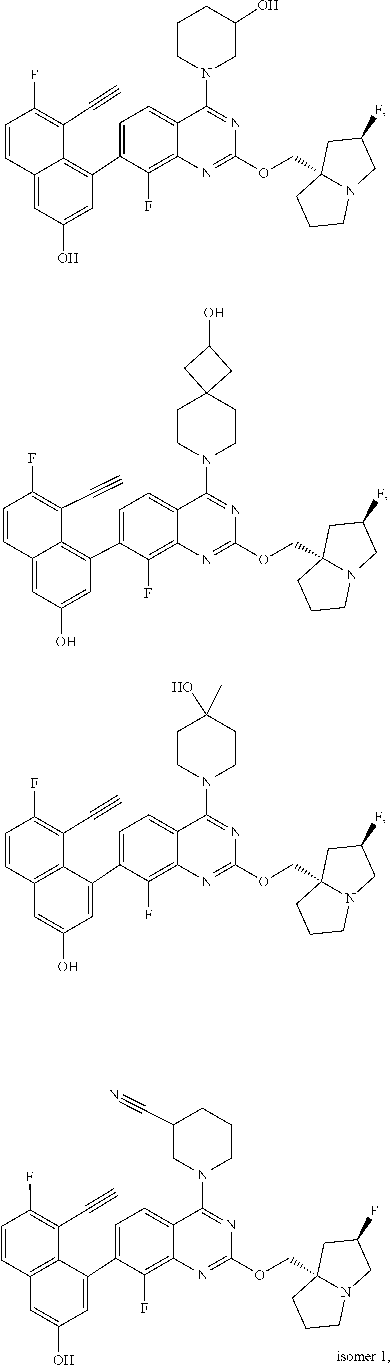 [SMILES: C#Cc1c(F)ccc2cc(O)cc(-c3ccc4c(N5CCCC(O)C5)nc(OC[C@@]56CCCN5C[C@H](F)C6)nc4c3F)c12 ; C#Cc1c(F)ccc2cc(O)cc(-c3ccc4c(N5CCC6(CC5)CC(O)C6)nc(OC[C@@]56CCCN5C[C@H](F)C6)nc4c3F)c12 ; C#Cc1c(F)ccc2cc(O)cc(-c3ccc4c(N5CCC(C)(O)CC5)nc(OC[C@@]56CCCN5C[C@H](F)C6)nc4c3F)c12 ; C#Cc1c(F)ccc2cc(O)cc(-c3ccc4c(N5CCCC(C#N)C5)nc(OC[C@@]56CCCN5C[C@H](F)C6)nc4c3F)c12]

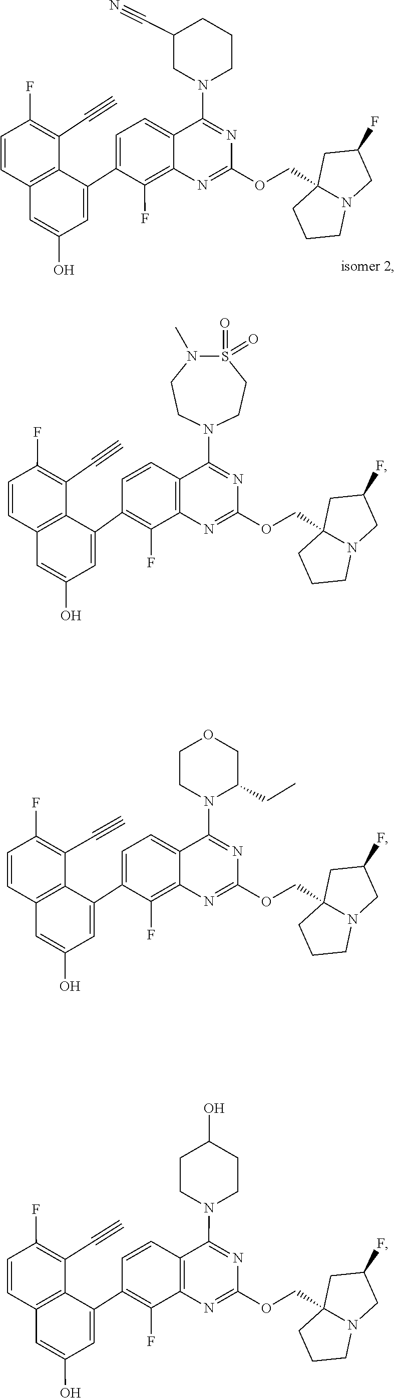 [SMILES: C#Cc1c(F)ccc2cc(O)cc(-c3ccc4c(N5CCCC(C#N)C5)nc(OC[C@@]56CCCN5C[C@H](F)C6)nc4c3F)c12 ; C#Cc1c(F)ccc2cc(O)cc(-c3ccc4c(N5CCN(C)S(=O)(=O)CC5)nc(OC[C@@]56CCCN5C[C@H](F)C6)nc4c3F)c12 ; C#Cc1c(F)ccc2cc(O)cc(-c3ccc4c(N5CCOC[C@@H]5CC)nc(OC[C@@]56CCCN5C[C@H](F)C6)nc4c3F)c12 ; C#Cc1c(F)ccc2cc(O)cc(-c3ccc4c(N5CCC(O)CC5)nc(OC[C@@]56CCCN5C[C@H](F)C6)nc4c3F)c12]

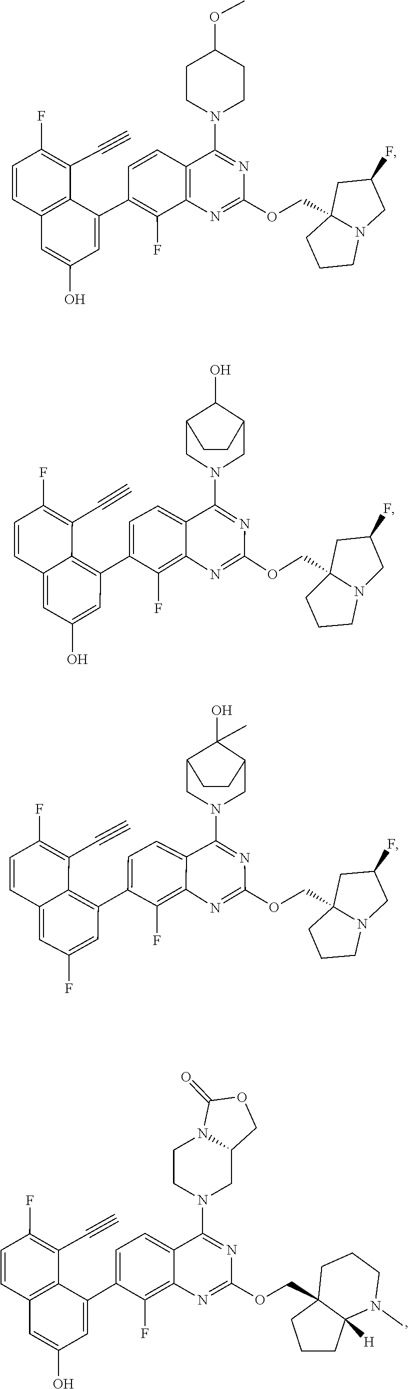 [SMILES: C#Cc1c(F)ccc2cc(O)cc(-c3ccc4c(N5CCC(OC)CC5)nc(OC[C@@]56CCCN5C[C@H](F)C6)nc4c3F)c12 ; C#Cc1c(F)ccc2cc(O)cc(-c3ccc4c(N5CC6CCC(C5)C6O)nc(OC[C@@]56CCCN5C[C@H](F)C6)nc4c3F)c12 ; C#Cc1c(F)ccc2cc(F)cc(-c3ccc4c(N5CC6CCC(C5)C6(C)O)nc(OC[C@@]56CCCN5C[C@H](F)C6)nc4c3F)c12 ; C#Cc1c(F)ccc2cc(O)cc(-c3ccc4c(N5CCN6C(=O)OC[C@@H]6C5)nc(OC[C@]56CCC[C@H]5N(C)CCC6)nc4c3F)c12]

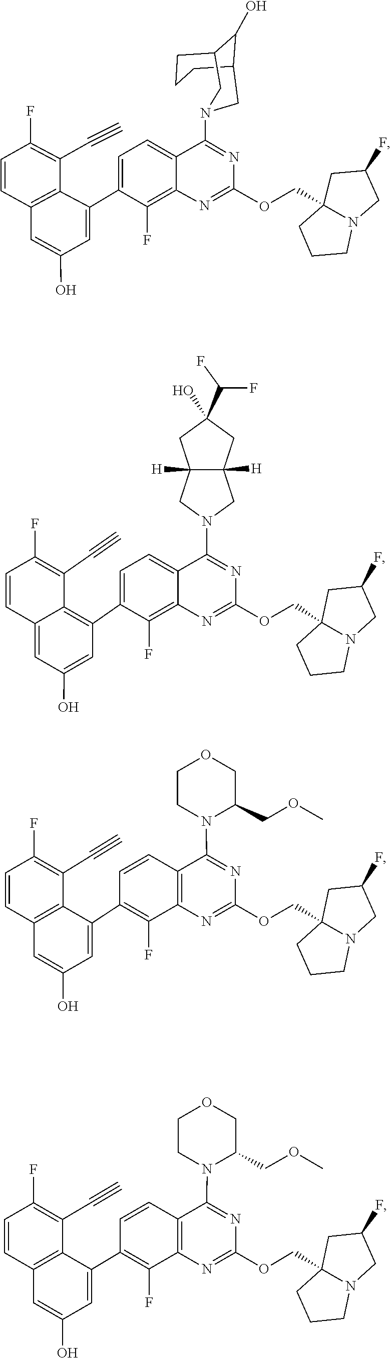 [SMILES: C#Cc1c(F)ccc2cc(O)cc(-c3ccc4c(N5CC6CCCC(C5)C6O)nc(OC[C@@]56CCCN5C[C@H](F)C6)nc4c3F)c12 ; C#Cc1c(F)ccc2cc(O)cc(-c3ccc4c(N5C[C@@H]6C[C@@](O)(C(F)F)C[C@@H]6C5)nc(OC[C@@]56CCCN5C[C@H](F)C6)nc4c3F)c12 ; C#Cc1c(F)ccc2cc(O)cc(-c3ccc4c(N5CCOC[C@H]5COC)nc(OC[C@@]56CCCN5C[C@H](F)C6)nc4c3F)c12 ; C#Cc1c(F)ccc2cc(O)cc(-c3ccc4c(N5CCOC[C@@H]5COC)nc(OC[C@@]56CCCN5C[C@H](F)C6)nc4c3F)c12]

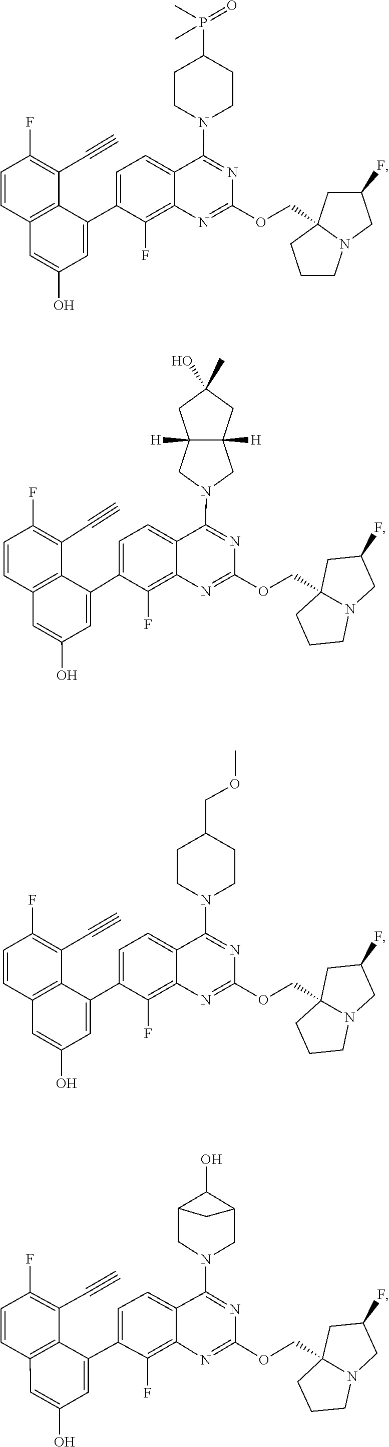 [SMILES: C#Cc1c(F)ccc2cc(O)cc(-c3ccc4c(N5CCC(P(C)(C)=O)CC5)nc(OC[C@@]56CCCN5C[C@H](F)C6)nc4c3F)c12 ; C#Cc1c(F)ccc2cc(O)cc(-c3ccc4c(N5C[C@@H]6C[C@](C)(O)C[C@@H]6C5)nc(OC[C@@]56CCCN5C[C@H](F)C6)nc4c3F)c12 ; C#Cc1c(F)ccc2cc(O)cc(-c3ccc4c(N5CCC(COC)CC5)nc(OC[C@@]56CCCN5C[C@H](F)C6)nc4c3F)c12 ; C#Cc1c(F)ccc2cc(O)cc(-c3ccc4c(N5CC6CC(C5)C6O)nc(OC[C@@]56CCCN5C[C@H](F)C6)nc4c3F)c12]

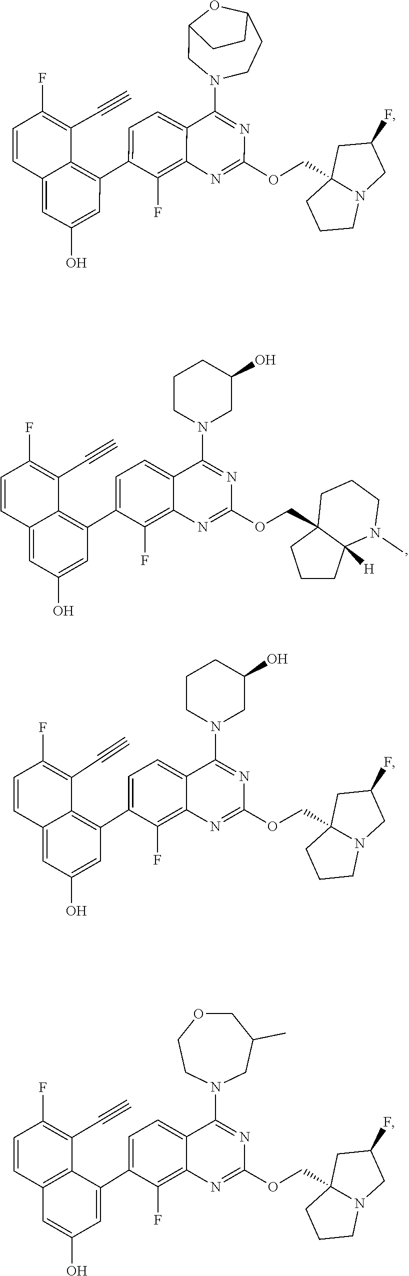 [SMILES: C#Cc1c(F)ccc2cc(O)cc(-c3ccc4c(N5CCC6CCC(C5)O6)nc(OC[C@@]56CCCN5C[C@H](F)C6)nc4c3F)c12 ; C#Cc1c(F)ccc2cc(O)cc(-c3ccc4c(N5CCC[C@@H](O)C5)nc(OC[C@]56CCC[C@H]5N(C)CCC6)nc4c3F)c12 ; C#Cc1c(F)ccc2cc(O)cc(-c3ccc4c(N5CCC[C@@H](O)C5)nc(OC[C@@]56CCCN5C[C@H](F)C6)nc4c3F)c12 ; C#Cc1c(F)ccc2cc(O)cc(-c3ccc4c(N5CCOCC(C)C5)nc(OC[C@@]56CCCN5C[C@H](F)C6)nc4c3F)c12]

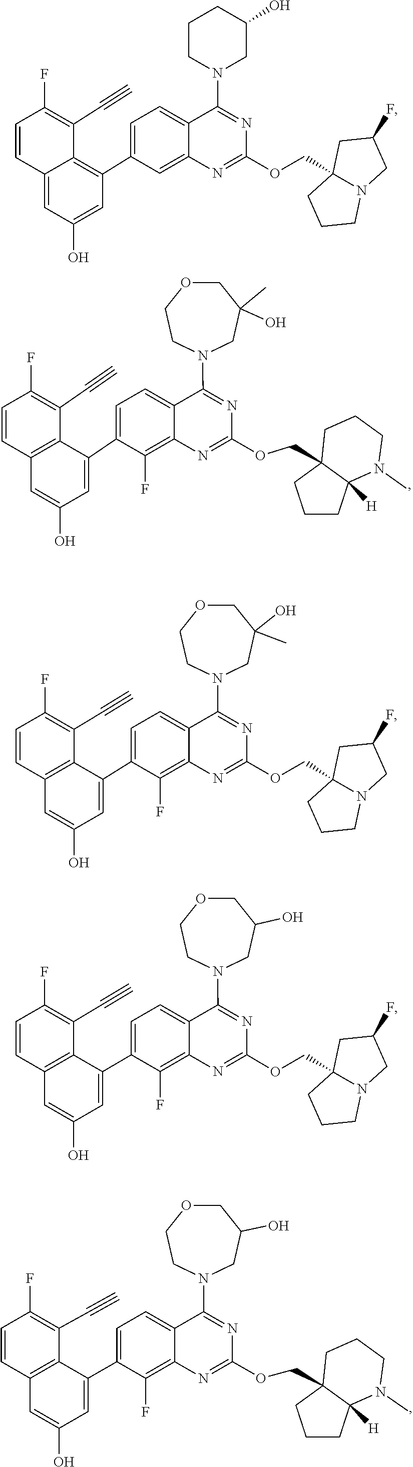 [SMILES: C#Cc1c(F)ccc2cc(O)cc(-c3ccc4c(N5CCC[C@H](O)C5)nc(OC[C@@]56CCCN5C[C@H](F)C6)nc4c3)c12 ; C#Cc1c(F)ccc2cc(O)cc(-c3ccc4c(N5CCOCC(C)(O)C5)nc(OC[C@]56CCC[C@H]5N(C)CCC6)nc4c3F)c12 ; C#Cc1c(F)ccc2cc(O)cc(-c3ccc4c(N5CCOCC(C)(O)C5)nc(OC[C@@]56CCCN5C[C@H](F)C6)nc4c3F)c12 ; C#Cc1c(F)ccc2cc(O)cc(-c3ccc4c(N5CCOCC(O)C5)nc(OC[C@@]56CCCN5C[C@H](F)C6)nc4c3F)c12 ; C#Cc1c(F)ccc2cc(O)cc(-c3ccc4c(N5CCOCC(O)C5)nc(OC[C@]56CCC[C@H]5N(C)CCC6)nc4c3F)c12]

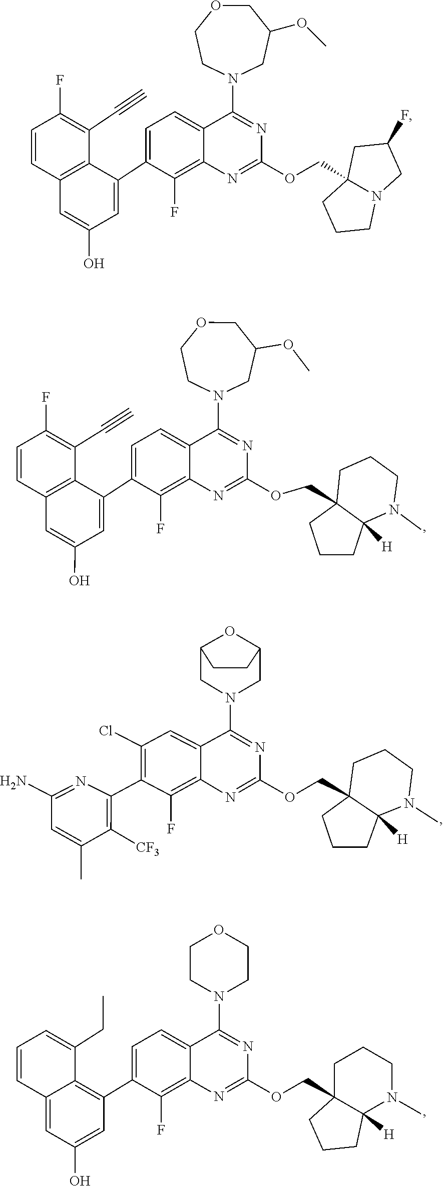 [SMILES: C#Cc1c(F)ccc2cc(O)cc(-c3ccc4c(N5CCOCC(OC)C5)nc(OC[C@@]56CCCN5C[C@H](F)C6)nc4c3F)c12 ; C#Cc1c(F)ccc2cc(O)cc(-c3ccc4c(N5CCOCC(OC)C5)nc(OC[C@]56CCC[C@H]5N(C)CCC6)nc4c3F)c12 ; Cc1cc(N)nc(-c2c(Cl)cc3c(N4CC5CCC(C4)O5)nc(OC[C@]45CCC[C@H]4N(C)CCC5)nc3c2F)c1C(F)(F)F ; CCc1cccc2cc(O)cc(-c3ccc4c(N5CCOCC5)nc(OC[C@]56CCC[C@H]5N(C)CCC6)nc4c3F)c12]

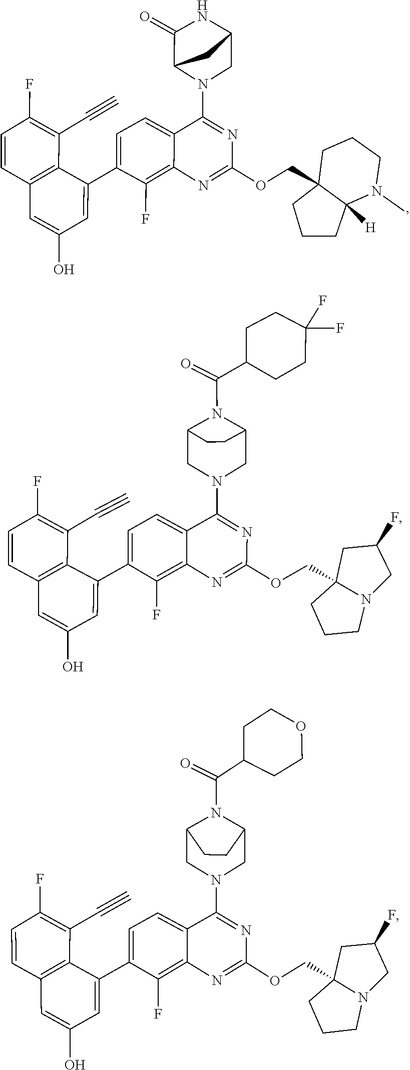 [SMILES: C#Cc1c(F)ccc2cc(O)cc(-c3ccc4c(N5C[C@@H]6C[C@H]5C(=O)N6)nc(OC[C@]56CCC[C@H]5N(C)CCC6)nc4c3F)c12 ; C#Cc1c(F)ccc2cc(O)cc(-c3ccc4c(N5CC6CCC(C5)N6C(=O)C5CCC(F)(F)CC5)nc(OC[C@@]56CCCN5C[C@H](F)C6)nc4c3F)c12 ; C#Cc1c(F)ccc2cc(O)cc(-c3ccc4c(N5CC6CCC(C5)N6C(=O)C5CCOCC5)nc(OC[C@@]56CCCN5C[C@H](F)C6)nc4c3F)c12]

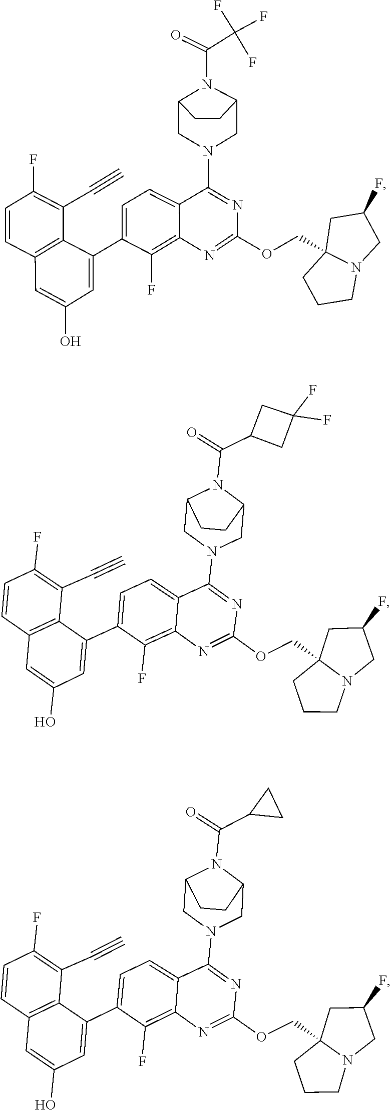 [SMILES: C#Cc1c(F)ccc2cc(O)cc(-c3ccc4c(N5CC6CCC(C5)N6C(=O)C(F)(F)F)nc(OC[C@@]56CCCN5C[C@H](F)C6)nc4c3F)c12 ; C#Cc1c(F)ccc2cc(O)cc(-c3ccc4c(N5CC6CCC(C5)N6C(=O)C5CC(F)(F)C5)nc(OC[C@@]56CCCN5C[C@H](F)C6)nc4c3F)c12 ; C#Cc1c(F)ccc2cc(O)cc(-c3ccc4c(N5CC6CCC(C5)N6C(=O)C5CC5)nc(OC[C@@]56CCCN5C[C@H](F)C6)nc4c3F)c12]

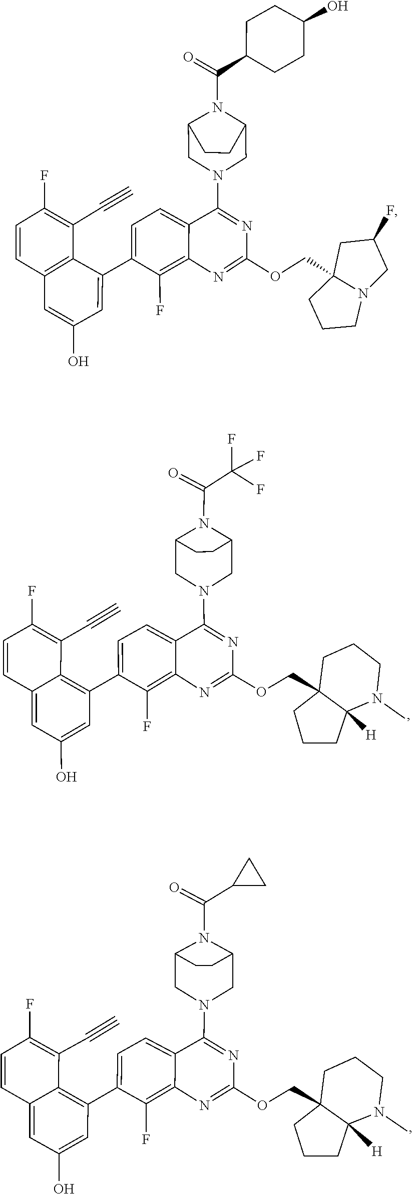 [SMILES: C#Cc1c(F)ccc2cc(O)cc(-c3ccc4c(N5CC6CCC(C5)N6C(=O)[C@H]5CC[C@@H](O)CC5)nc(OC[C@@]56CCCN5C[C@H](F)C6)nc4c3F)c12 ; C#Cc1c(F)ccc2cc(O)cc(-c3ccc4c(N5CC6CCC(C5)N6C(=O)C(F)(F)F)nc(OC[C@]56CCC[C@H]5N(C)CCC6)nc4c3F)c12 ; C#Cc1c(F)ccc2cc(O)cc(-c3ccc4c(N5CC6CCC(C5)N6C(=O)C5CC5)nc(OC[C@]56CCC[C@H]5N(C)CCC6)nc4c3F)c12]

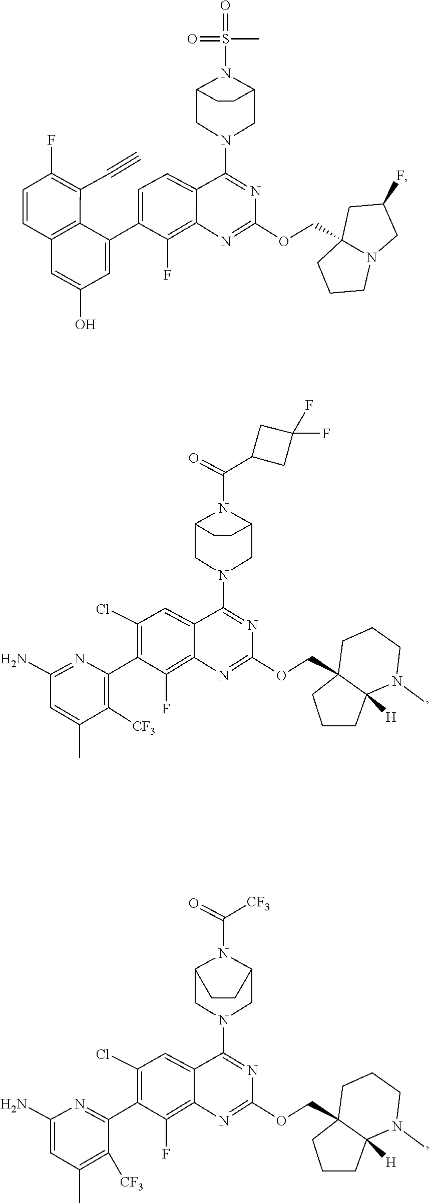 [SMILES: C#Cc1c(F)ccc2cc(O)cc(-c3ccc4c(N5CC6CCC(C5)N6S(C)(=O)=O)nc(OC[C@@]56CCCN5C[C@H](F)C6)nc4c3F)c12 ; Cc1cc(N)nc(-c2c(Cl)cc3c(N4CC5CCC(C4)N5C(=O)C4CC(F)(F)C4)nc(OC[C@]45CCC[C@H]4N(C)CCC5)nc3c2F)c1C(F)(F)F ; Cc1cc(N)nc(-c2c(Cl)cc3c(N4CC5CCC(C4)N5C(=O)C(F)(F)F)nc(OC[C@]45CCC[C@H]4N(C)CCC5)nc3c2F)c1C(F)(F)F]

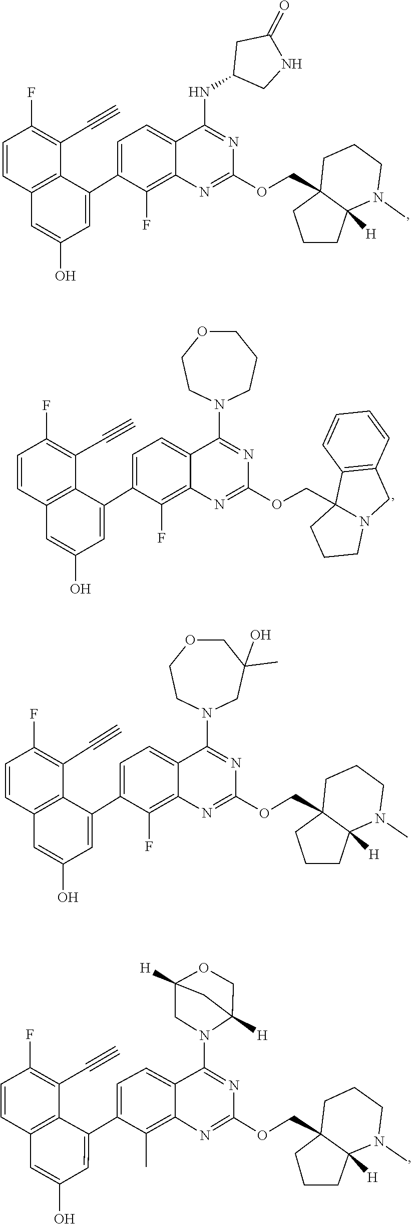 [SMILES: C#Cc1c(F)ccc2cc(O)cc(-c3ccc4c(N[C@H]5CNC(=O)C5)nc(OC[C@]56CCC[C@H]5N(C)CCC6)nc4c3F)c12 ; C#Cc1c(F)ccc2cc(O)cc(-c3ccc4c(N5CCCOCC5)nc(OCC56CCCN5Cc5ccccc56)nc4c3F)c12 ; C#Cc1c(F)ccc2cc(O)cc(-c3ccc4c(N5CCOCC(C)(O)C5)nc(OC[C@]56CCC[C@H]5N(C)CCC6)nc4c3F)c12 ; C#Cc1c(F)ccc2cc(O)cc(-c3ccc4c(N5C[C@H]6C[C@@H]5CO6)nc(OC[C@]56CCC[C@H]5N(C)CCC6)nc4c3C)c12]

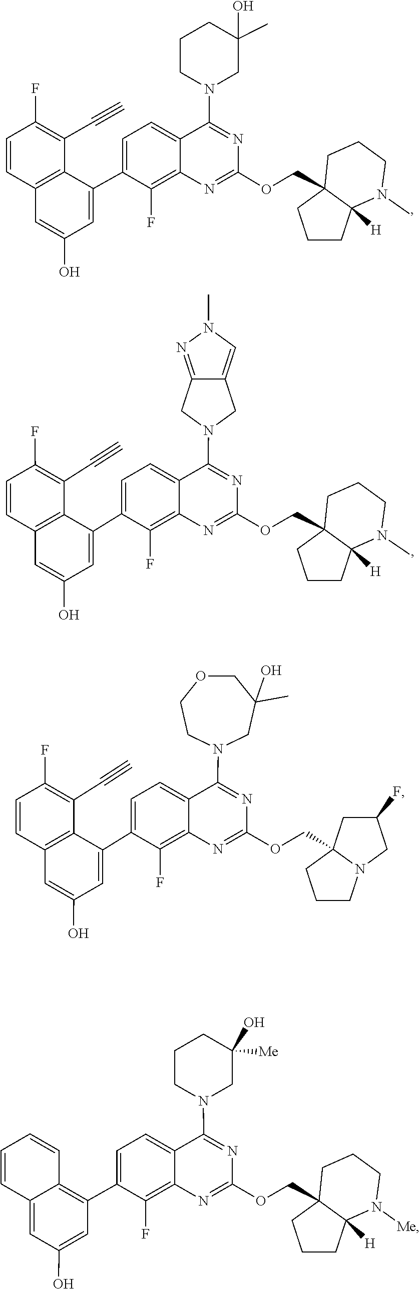 [SMILES: C#Cc1c(F)ccc2cc(O)cc(-c3ccc4c(N5CCCC(C)(O)C5)nc(OC[C@]56CCC[C@H]5N(C)CCC6)nc4c3F)c12 ; C#Cc1c(F)ccc2cc(O)cc(-c3ccc4c(N5Cc6cn(C)nc6C5)nc(OC[C@]56CCC[C@H]5N(C)CCC6)nc4c3F)c12 ; C#Cc1c(F)ccc2cc(O)cc(-c3ccc4c(N5CCOCC(C)(O)C5)nc(OC[C@@]56CCCN5C[C@H](F)C6)nc4c3F)c12 ; CN1CCC[C@@]2(COc3nc(N4CCC[C@@](C)(O)C4)c4ccc(-c5cc(O)cc6ccccc56)c(F)c4n3)CCC[C@@H]12]

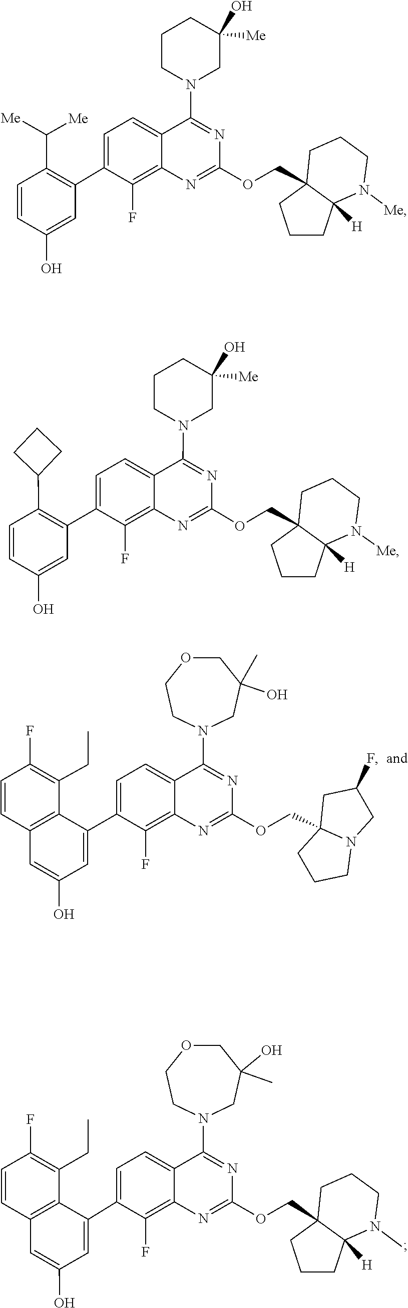 [SMILES: CC(C)c1ccc(O)cc1-c1ccc2c(N3CCC[C@@](C)(O)C3)nc(OC[C@]34CCC[C@H]3N(C)CCC4)nc2c1F ; CN1CCC[C@@]2(COc3nc(N4CCC[C@@](C)(O)C4)c4ccc(-c5cc(O)ccc5C5CCC5)c(F)c4n3)CCC[C@@H]12 ; CCc1c(F)ccc2cc(O)cc(-c3ccc4c(N5CCOCC(C)(O)C5)nc(OC[C@@]56CCCN5C[C@H](F)C6)nc4c3F)c12 ; CCc1c(F)ccc2cc(O)cc(-c3ccc4c(N5CCOCC(C)(O)C5)nc(OC[C@]56CCC[C@H]5N(C)CCC6)nc4c3F)c12]

or a pharmaceutically acceptable salt thereof.

In some aspects, the present disclosure provides a compound selected from the group consisting of:

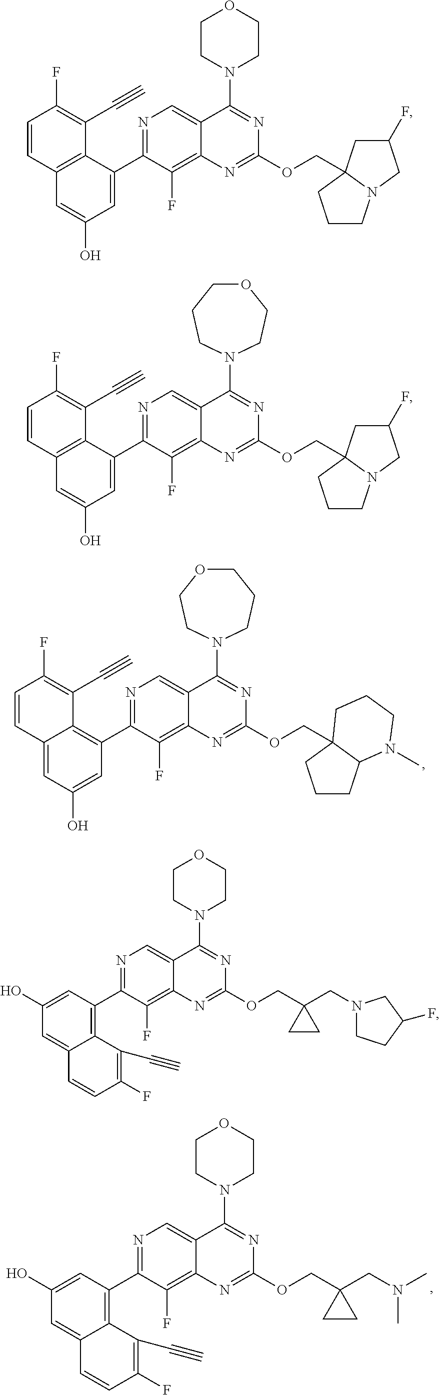 [SMILES: C#Cc1c(F)ccc2cc(O)cc(-c3ncc4c(N5CCOCC5)nc(OCC56CCCN5CC(F)C6)nc4c3F)c12 ; C#Cc1c(F)ccc2cc(O)cc(-c3ncc4c(N5CCCOCC5)nc(OCC56CCCN5CC(F)C6)nc4c3F)c12 ; C#Cc1c(F)ccc2cc(O)cc(-c3ncc4c(N5CCCOCC5)nc(OCC56CCCC5N(C)CCC6)nc4c3F)c12 ; C#Cc1c(F)ccc2cc(O)cc(-c3ncc4c(N5CCOCC5)nc(OCC5(CN6CCC(F)C6)CC5)nc4c3F)c12 ; C#Cc1c(F)ccc2cc(O)cc(-c3ncc4c(N5CCOCC5)nc(OCC5(CN(C)C)CC5)nc4c3F)c12]

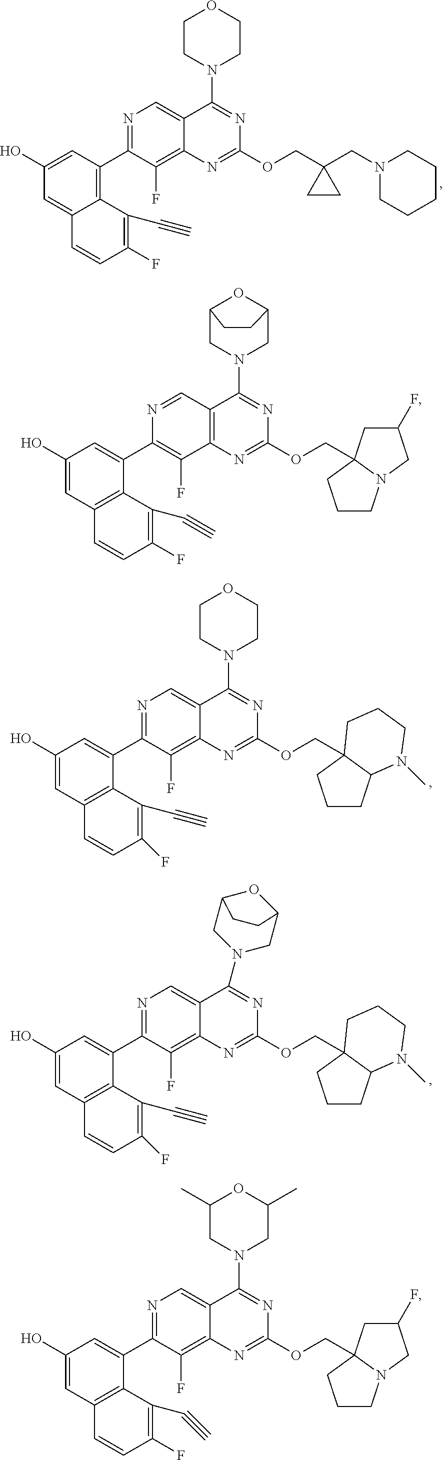

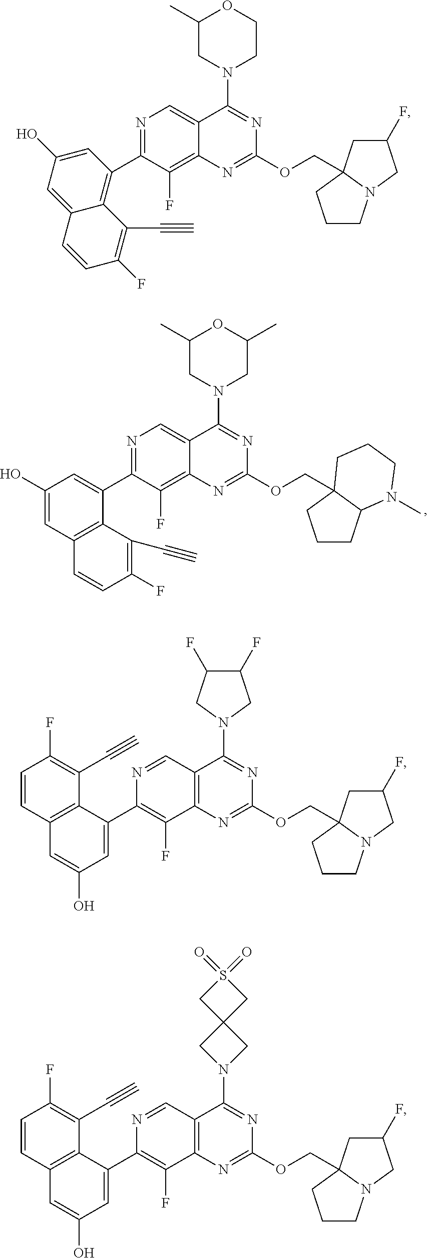

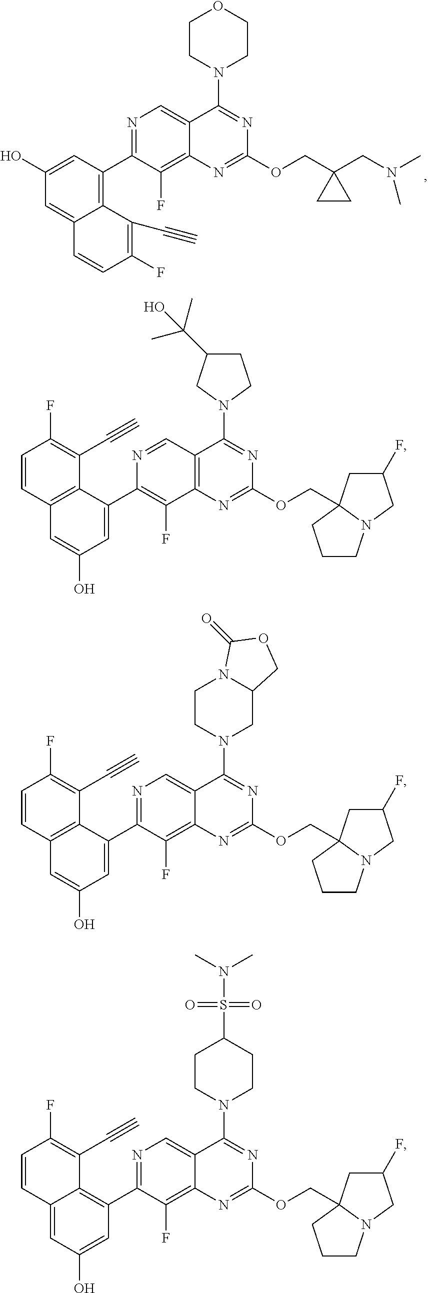

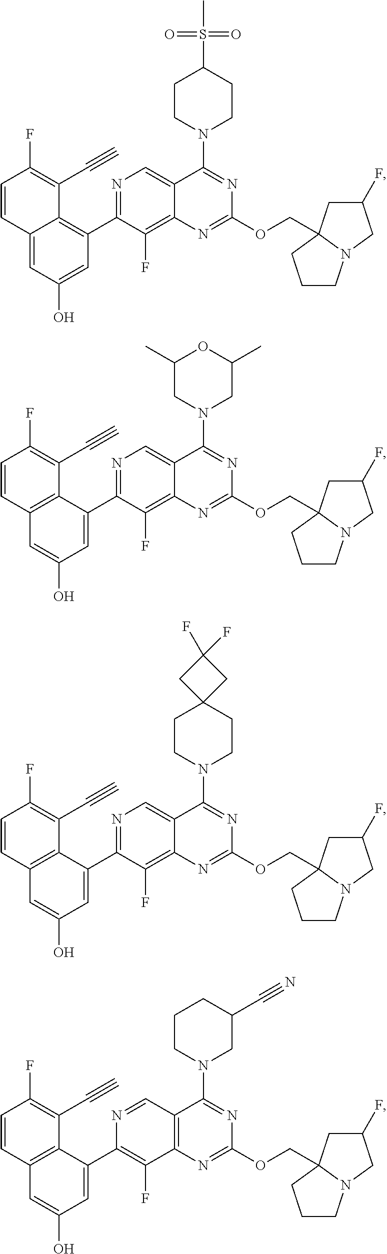 [SMILES: C#Cc1c(F)ccc2cc(O)cc(-c3ncc4c(N5CCC(S(C)(=O)=O)CC5)nc(OCC56CCCN5CC(F)C6)nc4c3F)c12 ; C#Cc1c(F)ccc2cc(O)cc(-c3ncc4c(N5CC(C)OC(C)C5)nc(OCC56CCCN5CC(F)C6)nc4c3F)c12 ; C#Cc1c(F)ccc2cc(O)cc(-c3ncc4c(N5CCC6(CC5)CC(F)(F)C6)nc(OCC56CCCN5CC(F)C6)nc4c3F)c12 ; C#Cc1c(F)ccc2cc(O)cc(-c3ncc4c(N5CCCC(C#N)C5)nc(OCC56CCCN5CC(F)C6)nc4c3F)c12]

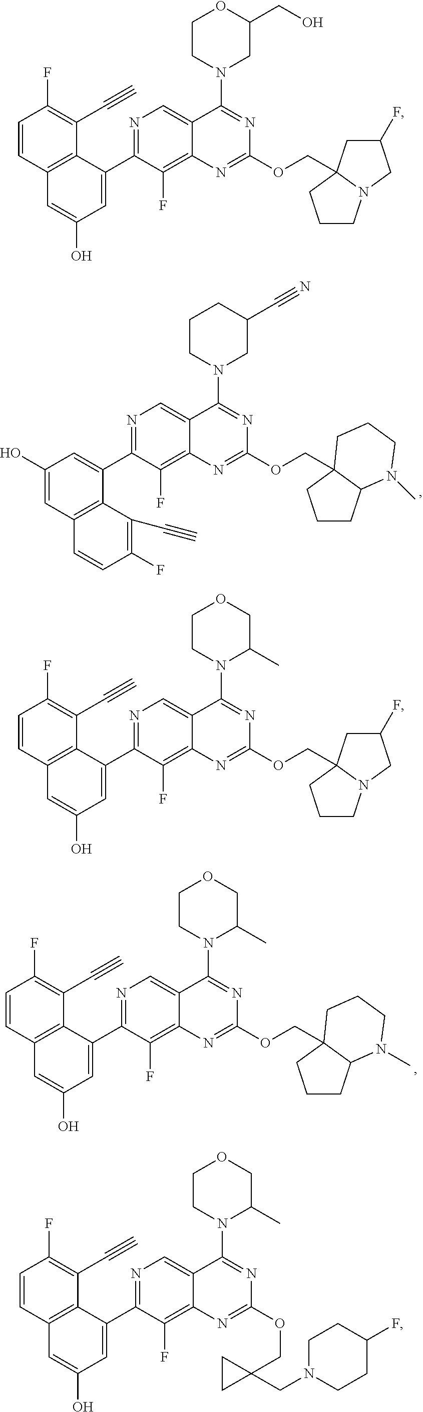 [SMILES: C#Cc1c(F)ccc2cc(O)cc(-c3ncc4c(N5CCOC(CO)C5)nc(OCC56CCCN5CC(F)C6)nc4c3F)c12 ; C#Cc1c(F)ccc2cc(O)cc(-c3ncc4c(N5CCCC(C#N)C5)nc(OCC56CCCC5N(C)CCC6)nc4c3F)c12 ; C#Cc1c(F)ccc2cc(O)cc(-c3ncc4c(N5CCOCC5C)nc(OCC56CCCN5CC(F)C6)nc4c3F)c12 ; C#Cc1c(F)ccc2cc(O)cc(-c3ncc4c(N5CCOCC5C)nc(OCC56CCCC5N(C)CCC6)nc4c3F)c12 ; C#Cc1c(F)ccc2cc(O)cc(-c3ncc4c(N5CCOCC5C)nc(OCC5(CN6CCC(F)CC6)CC5)nc4c3F)c12]

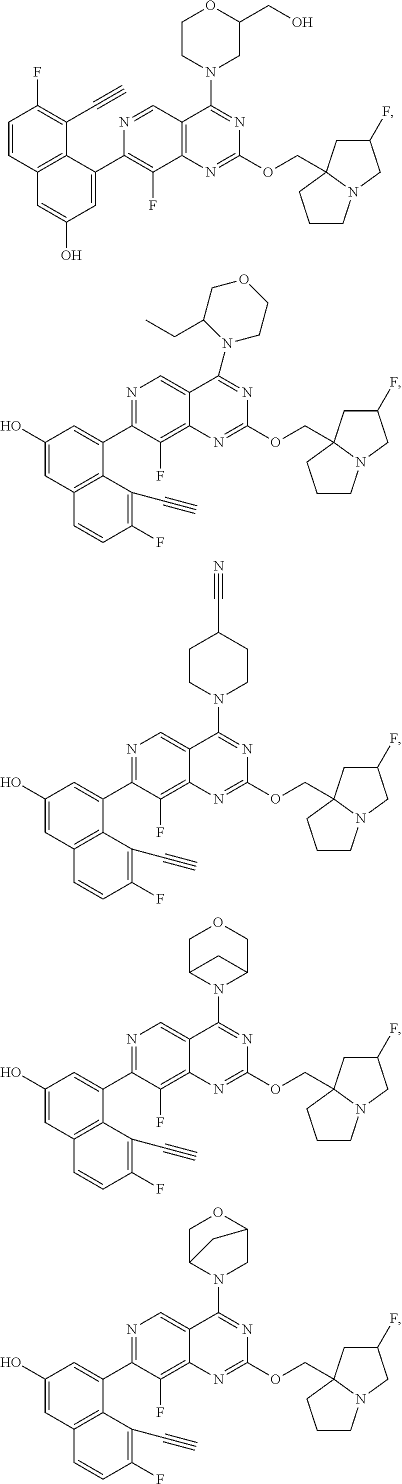 [SMILES: C#Cc1c(F)ccc2cc(O)cc(-c3ncc4c(N5CCOC(CO)C5)nc(OCC56CCCN5CC(F)C6)nc4c3F)c12 ; C#Cc1c(F)ccc2cc(O)cc(-c3ncc4c(N5CCOCC5CC)nc(OCC56CCCN5CC(F)C6)nc4c3F)c12 ; C#Cc1c(F)ccc2cc(O)cc(-c3ncc4c(N5CCC(C#N)CC5)nc(OCC56CCCN5CC(F)C6)nc4c3F)c12 ; C#Cc1c(F)ccc2cc(O)cc(-c3ncc4c(N5C6COCC5C6)nc(OCC56CCCN5CC(F)C6)nc4c3F)c12 ; C#Cc1c(F)ccc2cc(O)cc(-c3ncc4c(N5CC6CC5CO6)nc(OCC56CCCN5CC(F)C6)nc4c3F)c12]

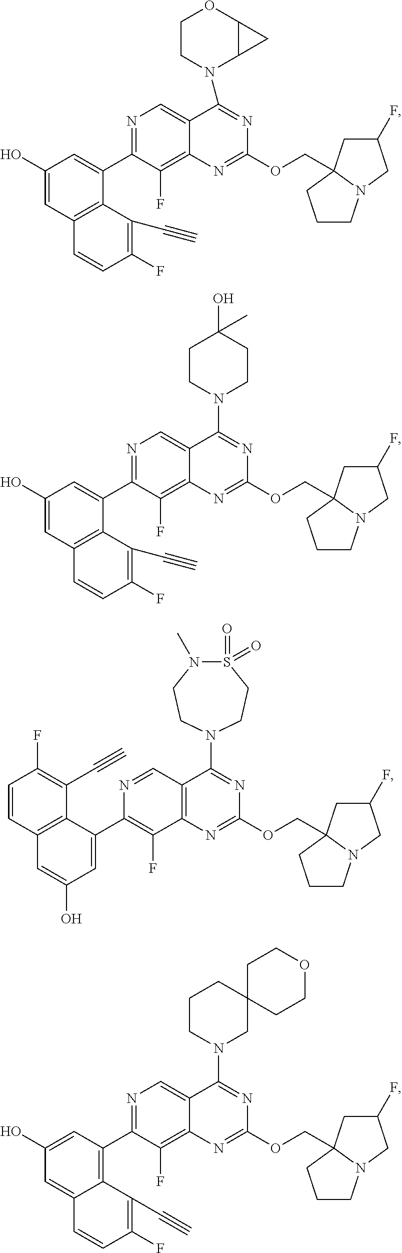

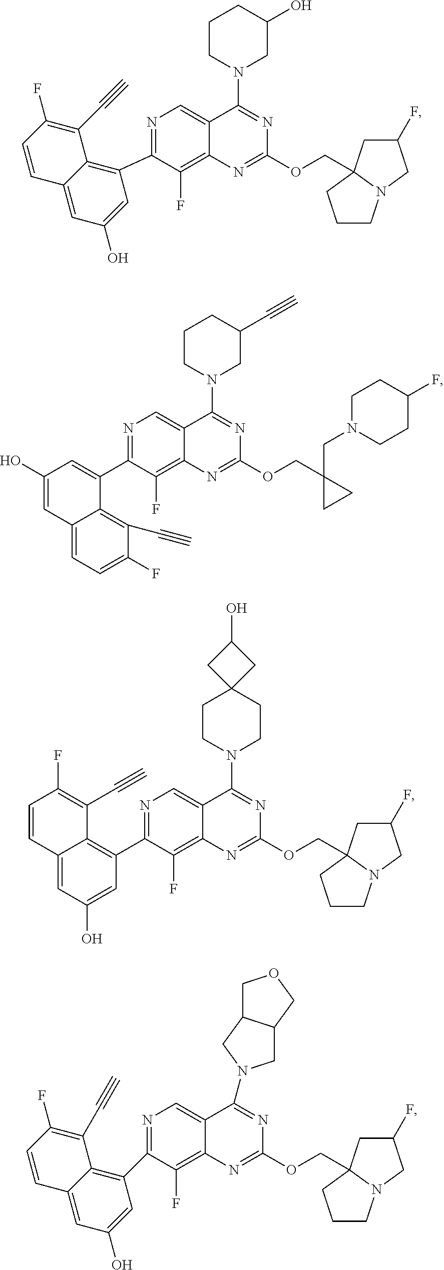 [SMILES: C#Cc1c(F)ccc2cc(O)cc(-c3ncc4c(N5CCCC(O)C5)nc(OCC56CCCN5CC(F)C6)nc4c3F)c12 ; C#Cc1c(F)ccc2cc(O)cc(-c3ncc4c(N5CCCC(C#C)C5)nc(OCC5(CN6CCC(F)CC6)CC5)nc4c3F)c12 ; C#Cc1c(F)ccc2cc(O)cc(-c3ncc4c(N5CCC6(CC5)CC(O)C6)nc(OCC56CCCN5CC(F)C6)nc4c3F)c12 ; C#Cc1c(F)ccc2cc(O)cc(-c3ncc4c(N5CC6COCC6C5)nc(OCC56CCCN5CC(F)C6)nc4c3F)c12]

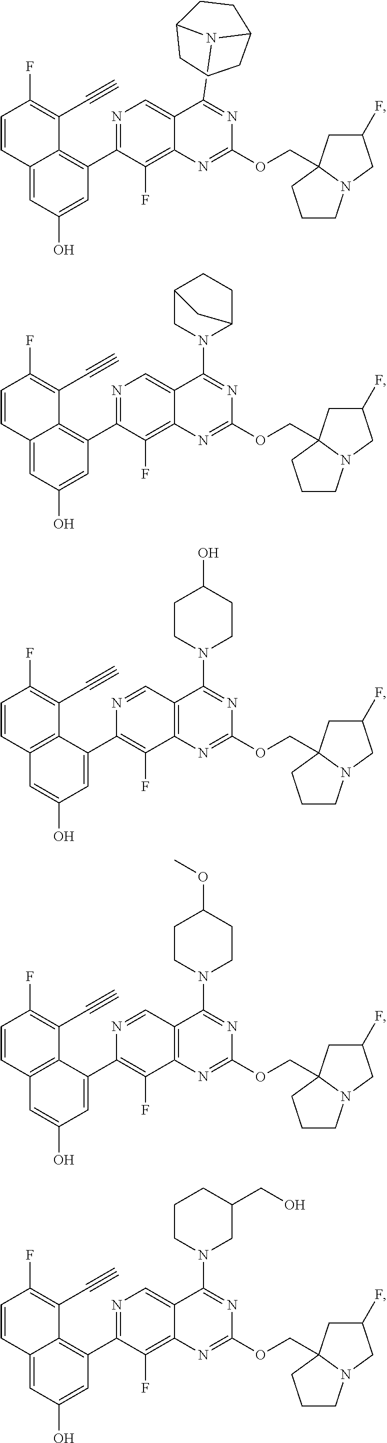 [SMILES: C#Cc1c(F)ccc2cc(O)cc(-c3ncc4c(N5C6CCCC5CC6)nc(OCC56CCCN5CC(F)C6)nc4c3F)c12 ; C#Cc1c(F)ccc2cc(O)cc(-c3ncc4c(N5CC6CCC5C6)nc(OCC56CCCN5CC(F)C6)nc4c3F)c12 ; C#Cc1c(F)ccc2cc(O)cc(-c3ncc4c(N5CCC(O)CC5)nc(OCC56CCCN5CC(F)C6)nc4c3F)c12 ; C#Cc1c(F)ccc2cc(O)cc(-c3ncc4c(N5CCC(OC)CC5)nc(OCC56CCCN5CC(F)C6)nc4c3F)c12 ; C#Cc1c(F)ccc2cc(O)cc(-c3ncc4c(N5CCCC(CO)C5)nc(OCC56CCCN5CC(F)C6)nc4c3F)c12]

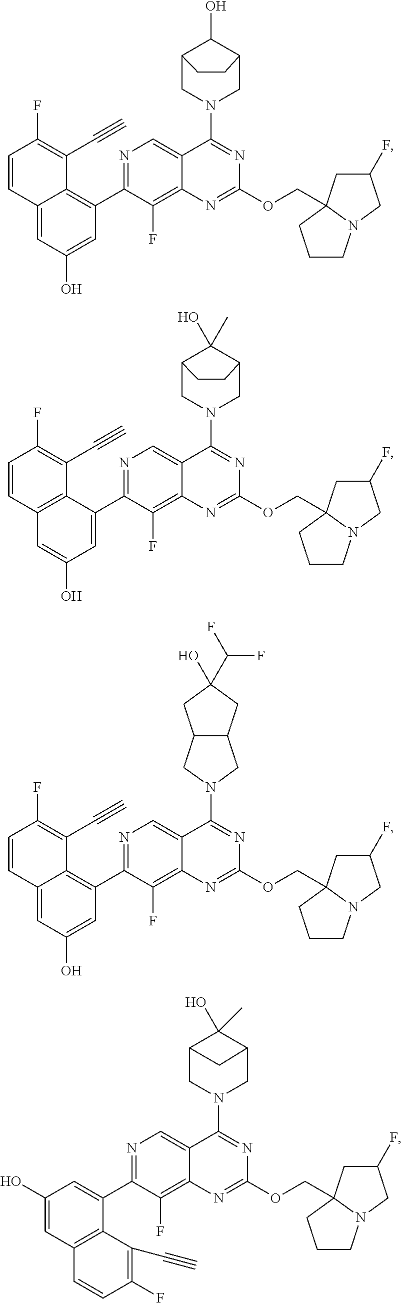 [SMILES: C#Cc1c(F)ccc2cc(O)cc(-c3ncc4c(N5CC6CCC(C5)C6O)nc(OCC56CCCN5CC(F)C6)nc4c3F)c12 ; C#Cc1c(F)ccc2cc(O)cc(-c3ncc4c(N5CC6CCC(C5)C6(C)O)nc(OCC56CCCN5CC(F)C6)nc4c3F)c12 ; C#Cc1c(F)ccc2cc(O)cc(-c3ncc4c(N5CC6CC(O)(C(F)F)CC6C5)nc(OCC56CCCN5CC(F)C6)nc4c3F)c12 ; C#Cc1c(F)ccc2cc(O)cc(-c3ncc4c(N5CC6CC(C5)C6(C)O)nc(OCC56CCCN5CC(F)C6)nc4c3F)c12]

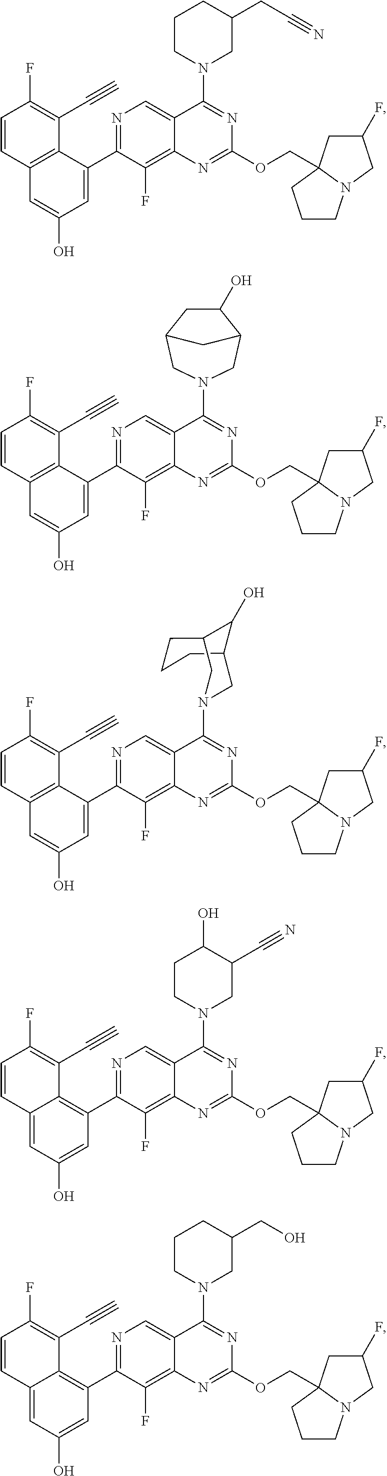 [SMILES: C#Cc1c(F)ccc2cc(O)cc(-c3ncc4c(N5CCCC(CC#N)C5)nc(OCC56CCCN5CC(F)C6)nc4c3F)c12 ; C#Cc1c(F)ccc2cc(O)cc(-c3ncc4c(N5CC6CC(O)C(C6)C5)nc(OCC56CCCN5CC(F)C6)nc4c3F)c12 ; C#Cc1c(F)ccc2cc(O)cc(-c3ncc4c(N5CC6CCCC(C5)C6O)nc(OCC56CCCN5CC(F)C6)nc4c3F)c12 ; C#Cc1c(F)ccc2cc(O)cc(-c3ncc4c(N5CCC(O)C(C#N)C5)nc(OCC56CCCN5CC(F)C6)nc4c3F)c12 ; C#Cc1c(F)ccc2cc(O)cc(-c3ncc4c(N5CCCC(CO)C5)nc(OCC56CCCN5CC(F)C6)nc4c3F)c12]

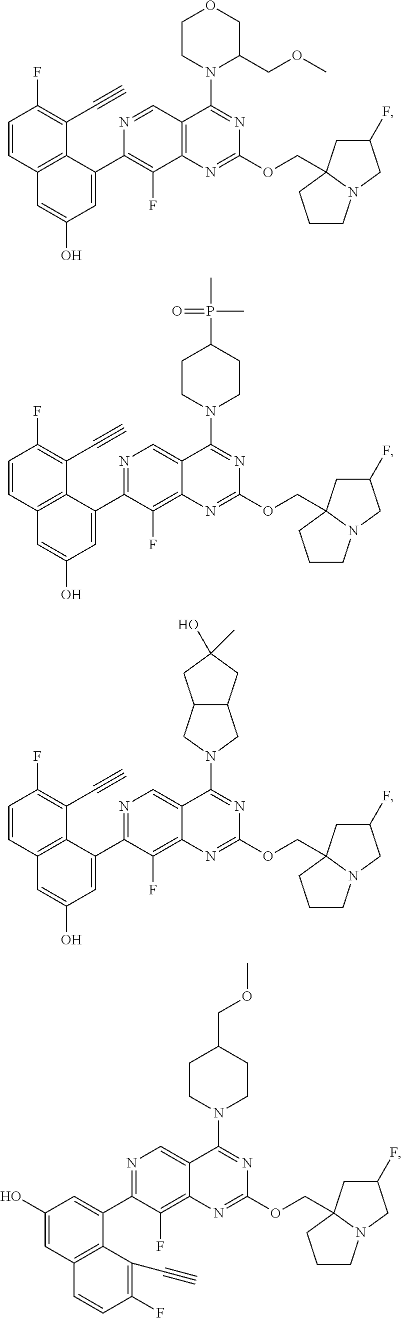 [SMILES: C#Cc1c(F)ccc2cc(O)cc(-c3ncc4c(N5CCOCC5COC)nc(OCC56CCCN5CC(F)C6)nc4c3F)c12 ; C#Cc1c(F)ccc2cc(O)cc(-c3ncc4c(N5CCC(P(C)(C)=O)CC5)nc(OCC56CCCN5CC(F)C6)nc4c3F)c12 ; C#Cc1c(F)ccc2cc(O)cc(-c3ncc4c(N5CC6CC(C)(O)CC6C5)nc(OCC56CCCN5CC(F)C6)nc4c3F)c12 ; C#Cc1c(F)ccc2cc(O)cc(-c3ncc4c(N5CCC(COC)CC5)nc(OCC56CCCN5CC(F)C6)nc4c3F)c12]

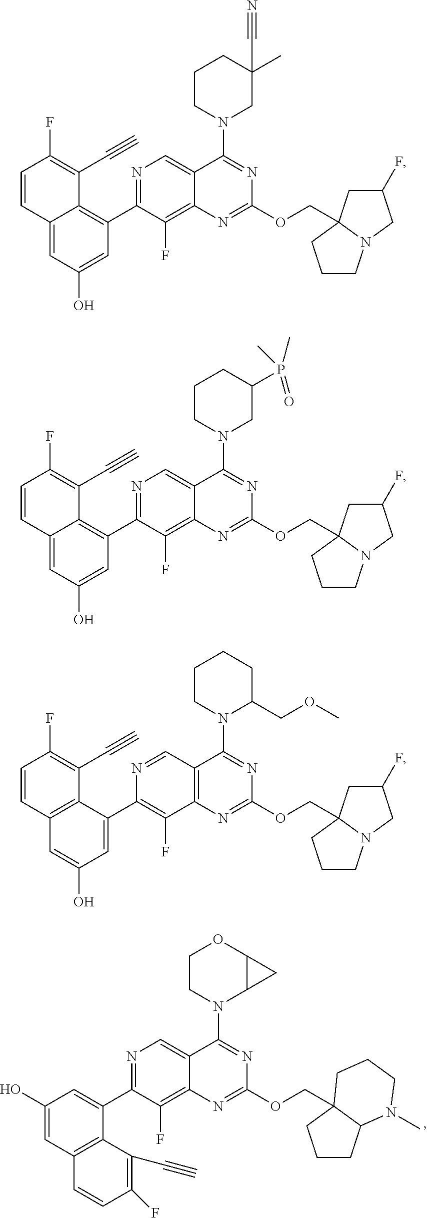 [SMILES: C#Cc1c(F)ccc2cc(O)cc(-c3ncc4c(N5CCCC(C)(C#N)C5)nc(OCC56CCCN5CC(F)C6)nc4c3F)c12 ; C#Cc1c(F)ccc2cc(O)cc(-c3ncc4c(N5CCCC(P(C)(C)=O)C5)nc(OCC56CCCN5CC(F)C6)nc4c3F)c12 ; C#Cc1c(F)ccc2cc(O)cc(-c3ncc4c(N5CCCCC5COC)nc(OCC56CCCN5CC(F)C6)nc4c3F)c12 ; C#Cc1c(F)ccc2cc(O)cc(-c3ncc4c(N5CCOC6CC65)nc(OCC56CCCC5N(C)CCC6)nc4c3F)c12]

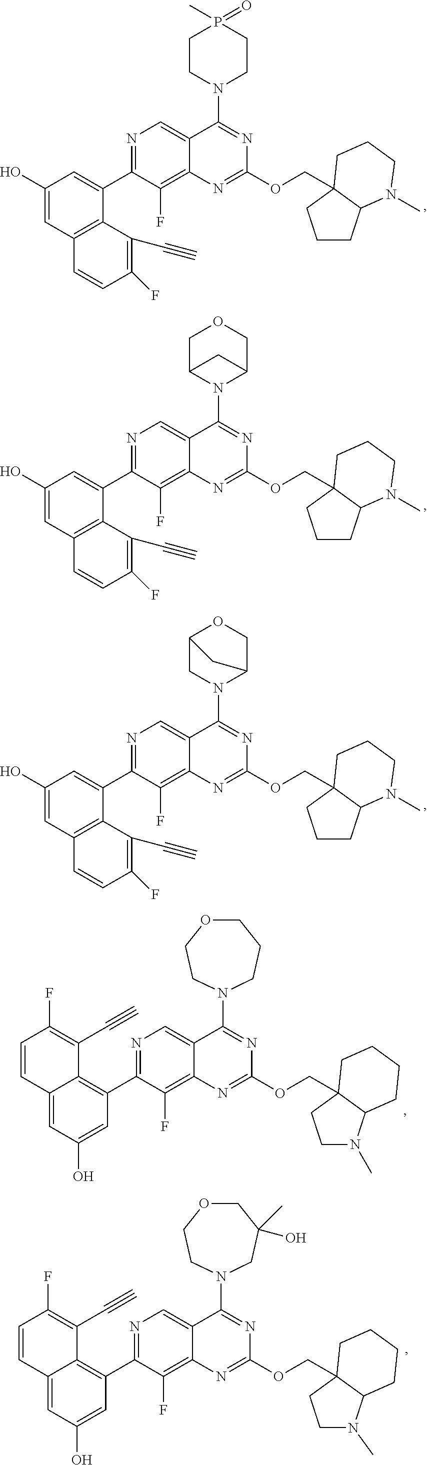 [SMILES: C#Cc1c(F)ccc2cc(O)cc(-c3ncc4c(N5CCP(C)(=O)CC5)nc(OCC56CCCC5N(C)CCC6)nc4c3F)c12 ; C#Cc1c(F)ccc2cc(O)cc(-c3ncc4c(N5C6COCC5C6)nc(OCC56CCCC5N(C)CCC6)nc4c3F)c12 ; C#Cc1c(F)ccc2cc(O)cc(-c3ncc4c(N5CC6CC5CO6)nc(OCC56CCCC5N(C)CCC6)nc4c3F)c12 ; C#Cc1c(F)ccc2cc(O)cc(-c3ncc4c(N5CCCOCC5)nc(OCC56CCCCC5N(C)CC6)nc4c3F)c12 ; C#Cc1c(F)ccc2cc(O)cc(-c3ncc4c(N5CCOCC(C)(O)C5)nc(OCC56CCCCC5N(C)CC6)nc4c3F)c12]

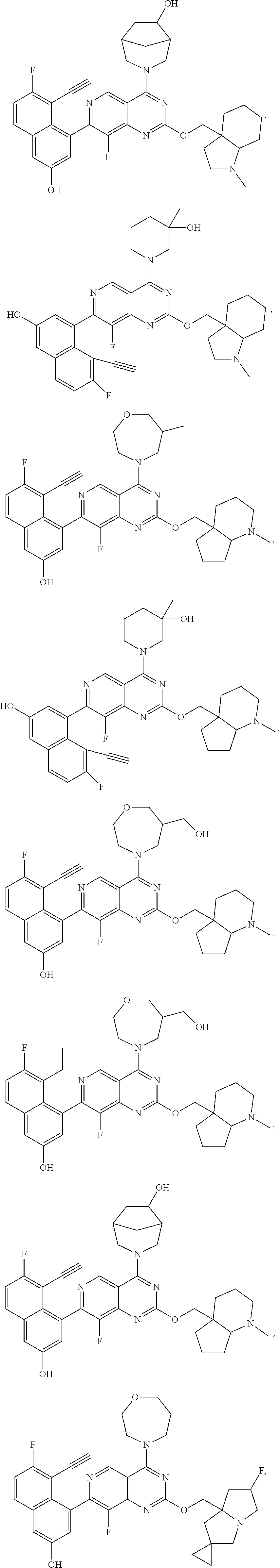

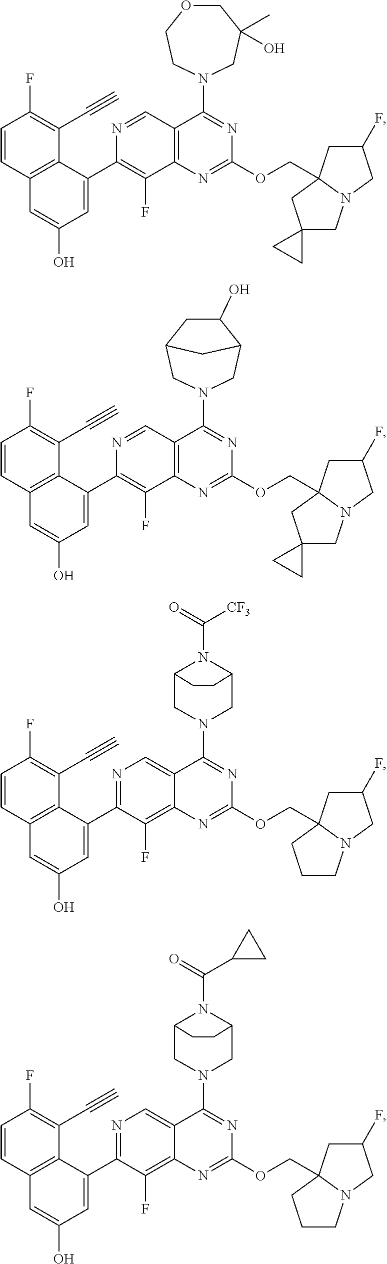 [SMILES: C#Cc1c(F)ccc2cc(O)cc(-c3ncc4c(N5CCOCC(C)(O)C5)nc(OCC56CC(F)CN5CC5(CC5)C6)nc4c3F)c12 ; C#Cc1c(F)ccc2cc(O)cc(-c3ncc4c(N5CC6CC(O)C(C6)C5)nc(OCC56CC(F)CN5CC5(CC5)C6)nc4c3F)c12 ; C#Cc1c(F)ccc2cc(O)cc(-c3ncc4c(N5CC6CCC(C5)N6C(=O)C(F)(F)F)nc(OCC56CCCN5CC(F)C6)nc4c3F)c12 ; C#Cc1c(F)ccc2cc(O)cc(-c3ncc4c(N5CC6CCC(C5)N6C(=O)C5CC5)nc(OCC56CCCN5CC(F)C6)nc4c3F)c12]

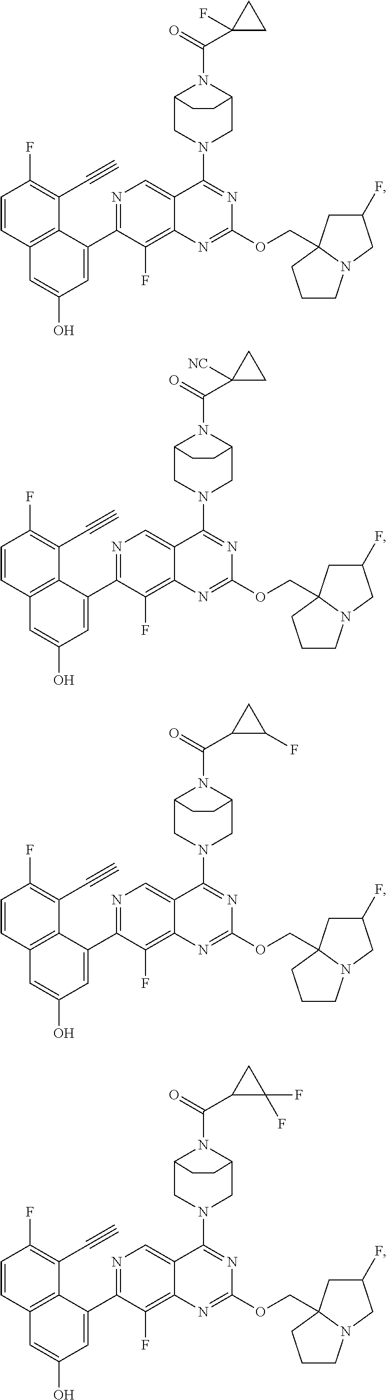 [SMILES: C#Cc1c(F)ccc2cc(O)cc(-c3ncc4c(N5CC6CCC(C5)N6C(=O)C5(F)CC5)nc(OCC56CCCN5CC(F)C6)nc4c3F)c12 ; C#Cc1c(F)ccc2cc(O)cc(-c3ncc4c(N5CC6CCC(C5)N6C(=O)C5(C#N)CC5)nc(OCC56CCCN5CC(F)C6)nc4c3F)c12 ; C#Cc1c(F)ccc2cc(O)cc(-c3ncc4c(N5CC6CCC(C5)N6C(=O)C5CC5F)nc(OCC56CCCN5CC(F)C6)nc4c3F)c12 ; C#Cc1c(F)ccc2cc(O)cc(-c3ncc4c(N5CC6CCC(C5)N6C(=O)C5CC5(F)F)nc(OCC56CCCN5CC(F)C6)nc4c3F)c12]

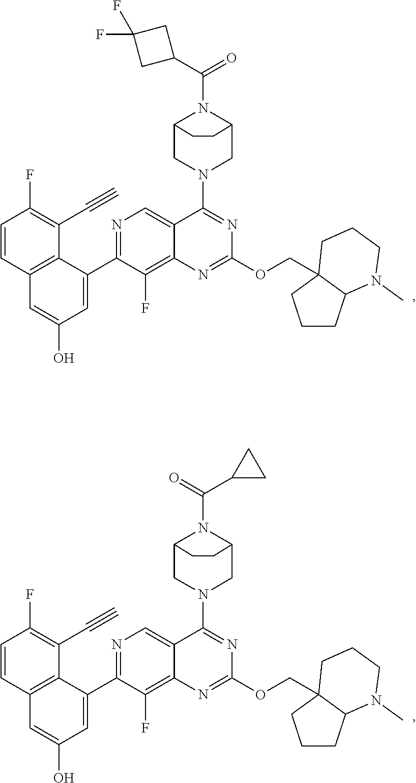 [SMILES: C#Cc1c(F)ccc2cc(O)cc(-c3ncc4c(N5CC6CCC(C5)N6C(=O)C5CC(F)(F)C5)nc(OCC56CCCC5N(C)CCC6)nc4c3F)c12 ; C#Cc1c(F)ccc2cc(O)cc(-c3ncc4c(N5CC6CCC(C5)N6C(=O)C5CC5)nc(OCC56CCCC5N(C)CCC6)nc4c3F)c12]

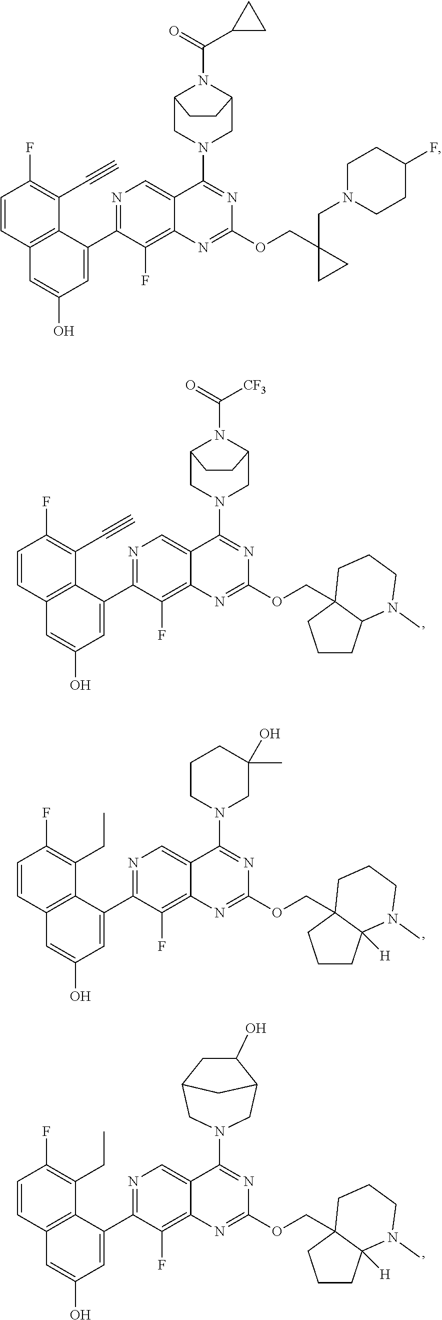 [SMILES: C#Cc1c(F)ccc2cc(O)cc(-c3ncc4c(N5CC6CCC(C5)N6C(=O)C5CC5)nc(OCC5(CN6CCC(F)CC6)CC5)nc4c3F)c12 ; C#Cc1c(F)ccc2cc(O)cc(-c3ncc4c(N5CC6CCC(C5)N6C(=O)C(F)(F)F)nc(OCC56CCCC5N(C)CCC6)nc4c3F)c12 ; CCc1c(F)ccc2cc(O)cc(-c3ncc4c(N5CCCC(C)(O)C5)nc(OCC56CCCC5N(C)CCC6)nc4c3F)c12 ; CCc1c(F)ccc2cc(O)cc(-c3ncc4c(N5CC6CC(O)C(C6)C5)nc(OCC56CCCC5N(C)CCC6)nc4c3F)c12]

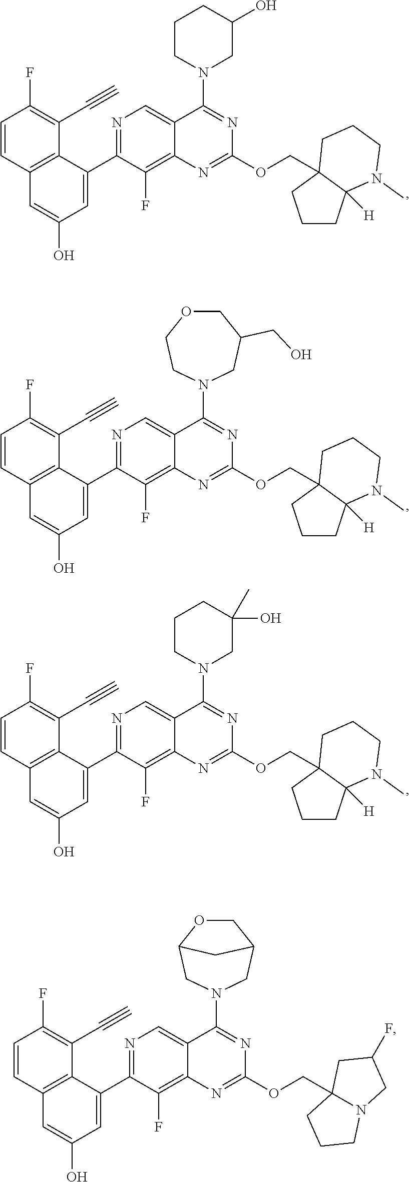 [SMILES: C#Cc1c(F)ccc2cc(O)cc(-c3ncc4c(N5CCCC(O)C5)nc(OCC56CCCC5N(C)CCC6)nc4c3F)c12 ; C#Cc1c(F)ccc2cc(O)cc(-c3ncc4c(N5CCOCC(CO)C5)nc(OCC56CCCC5N(C)CCC6)nc4c3F)c12 ; C#Cc1c(F)ccc2cc(O)cc(-c3ncc4c(N5CCCC(C)(O)C5)nc(OCC56CCCC5N(C)CCC6)nc4c3F)c12 ; C#Cc1c(F)ccc2cc(O)cc(-c3ncc4c(N5CC6COC(C6)C5)nc(OCC56CCCN5CC(F)C6)nc4c3F)c12]

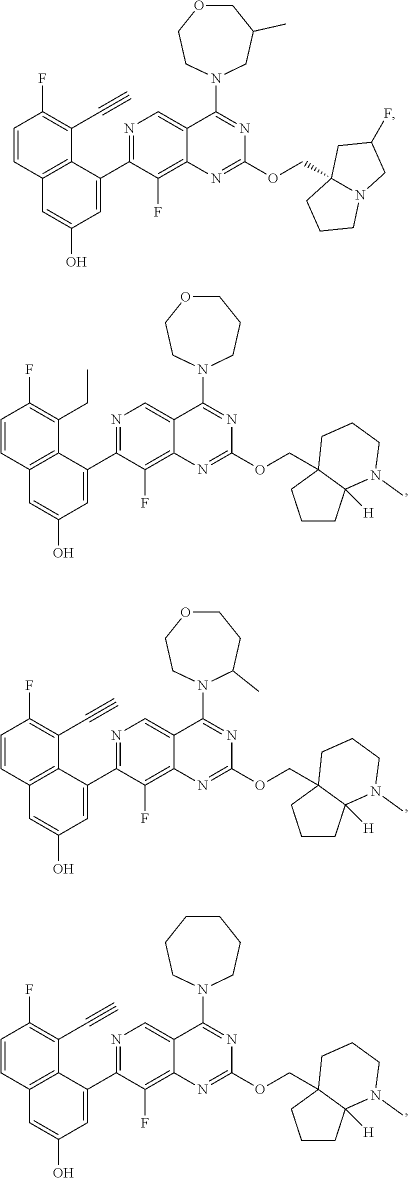 [SMILES: C#Cc1c(F)ccc2cc(O)cc(-c3ncc4c(N5CCOCC(C)C5)nc(OC[C@@]56CCCN5CC(F)C6)nc4c3F)c12 ; CCc1c(F)ccc2cc(O)cc(-c3ncc4c(N5CCCOCC5)nc(OCC56CCCC5N(C)CCC6)nc4c3F)c12 ; C#Cc1c(F)ccc2cc(O)cc(-c3ncc4c(N5CCOCCC5C)nc(OCC56CCCC5N(C)CCC6)nc4c3F)c12 ; C#Cc1c(F)ccc2cc(O)cc(-c3ncc4c(N5CCCCCC5)nc(OCC56CCCC5N(C)CCC6)nc4c3F)c12]

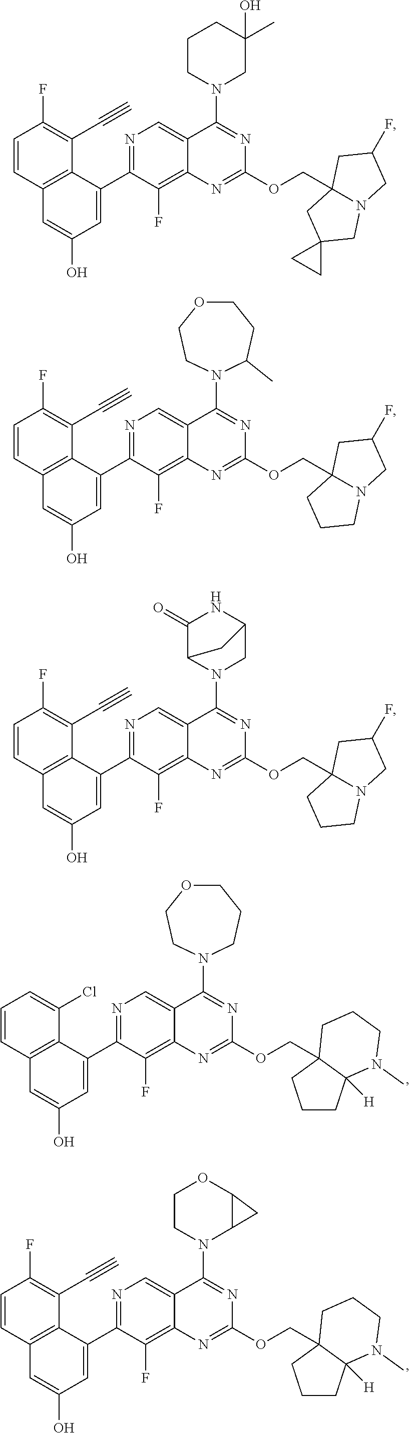 [SMILES: C#Cc1c(F)ccc2cc(O)cc(-c3ncc4c(N5CCCC(C)(O)C5)nc(OCC56CC(F)CN5CC5(CC5)C6)nc4c3F)c12 ; C#Cc1c(F)ccc2cc(O)cc(-c3ncc4c(N5CCOCCC5C)nc(OCC56CCCN5CC(F)C6)nc4c3F)c12 ; C#Cc1c(F)ccc2cc(O)cc(-c3ncc4c(N5CC6CC5C(=O)N6)nc(OCC56CCCN5CC(F)C6)nc4c3F)c12 ; CN1CCCC2(COc3nc(N4CCCOCC4)c4cnc(-c5cc(O)cc6cccc(Cl)c56)c(F)c4n3)CCCC12 ; C#Cc1c(F)ccc2cc(O)cc(-c3ncc4c(N5CCOC6CC65)nc(OCC56CCCC5N(C)CCC6)nc4c3F)c12]

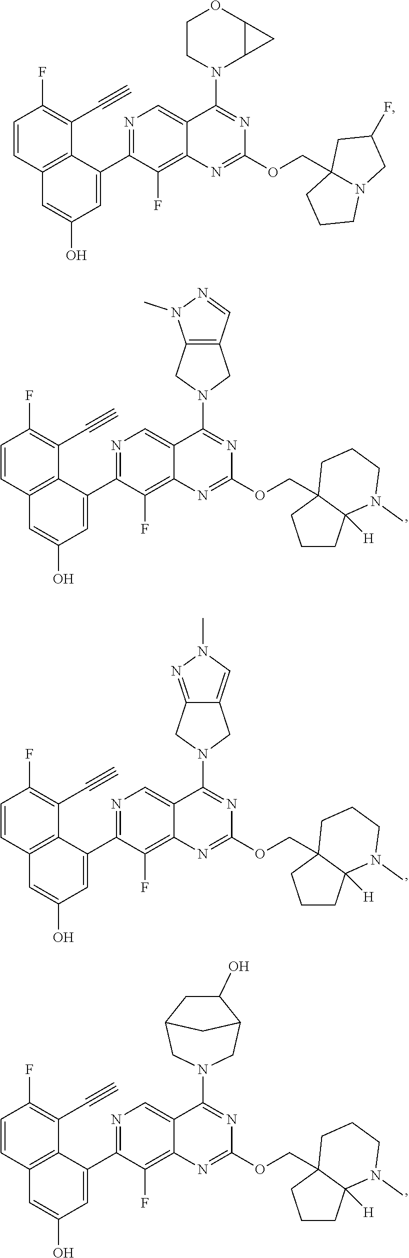 [SMILES: C#Cc1c(F)ccc2cc(O)cc(-c3ncc4c(N5CCOC6CC65)nc(OCC56CCCN5CC(F)C6)nc4c3F)c12 ; C#Cc1c(F)ccc2cc(O)cc(-c3ncc4c(N5Cc6cnn(C)c6C5)nc(OCC56CCCC5N(C)CCC6)nc4c3F)c12 ; C#Cc1c(F)ccc2cc(O)cc(-c3ncc4c(N5Cc6cn(C)nc6C5)nc(OCC56CCCC5N(C)CCC6)nc4c3F)c12 ; C#Cc1c(F)ccc2cc(O)cc(-c3ncc4c(N5CC6CC(O)C(C6)C5)nc(OCC56CCCC5N(C)CCC6)nc4c3F)c12]

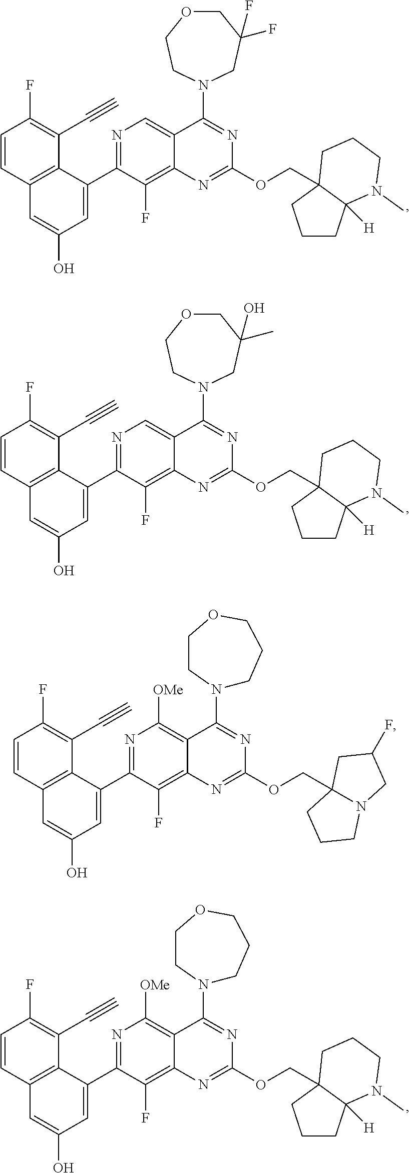 [SMILES: C#Cc1c(F)ccc2cc(O)cc(-c3ncc4c(N5CCOCC(F)(F)C5)nc(OCC56CCCC5N(C)CCC6)nc4c3F)c12 ; C#Cc1c(F)ccc2cc(O)cc(-c3ncc4c(N5CCOCC(C)(O)C5)nc(OCC56CCCC5N(C)CCC6)nc4c3F)c12 ; C#Cc1c(F)ccc2cc(O)cc(-c3nc(OC)c4c(N5CCCOCC5)nc(OCC56CCCN5CC(F)C6)nc4c3F)c12 ; C#Cc1c(F)ccc2cc(O)cc(-c3nc(OC)c4c(N5CCCOCC5)nc(OCC56CCCC5N(C)CCC6)nc4c3F)c12]

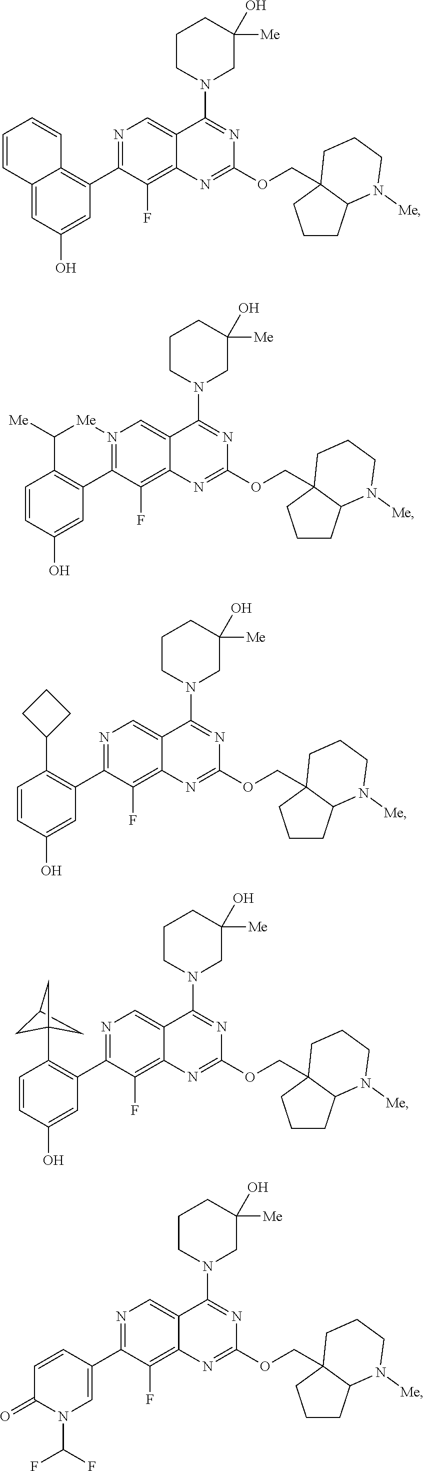

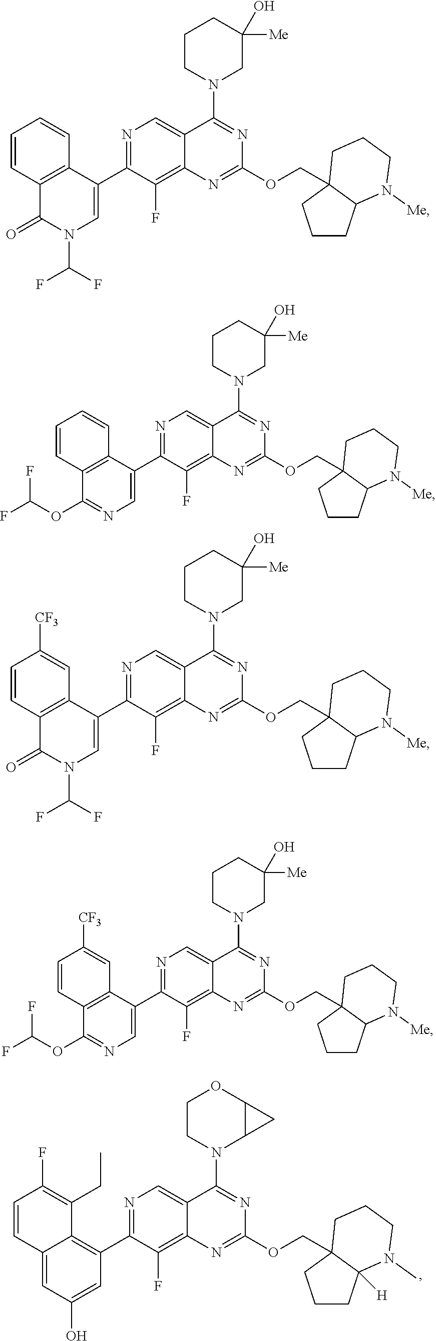 [SMILES: CN1CCCC2(COc3nc(N4CCCC(C)(O)C4)c4cnc(-c5cn(C(F)F)c(=O)c6ccccc56)c(F)c4n3)CCCC12 ; CN1CCCC2(COc3nc(N4CCCC(C)(O)C4)c4cnc(-c5cnc(OC(F)F)c6ccccc56)c(F)c4n3)CCCC12 ; CN1CCCC2(COc3nc(N4CCCC(C)(O)C4)c4cnc(-c5cn(C(F)F)c(=O)c6ccc(C(F)(F)F)cc56)c(F)c4n3)CCCC12 ; CN1CCCC2(COc3nc(N4CCCC(C)(O)C4)c4cnc(-c5cnc(OC(F)F)c6ccc(C(F)(F)F)cc56)c(F)c4n3)CCCC12 ; CCc1c(F)ccc2cc(O)cc(-c3ncc4c(N5CCOC6CC65)nc(OCC56CCCC5N(C)CCC6)nc4c3F)c12]

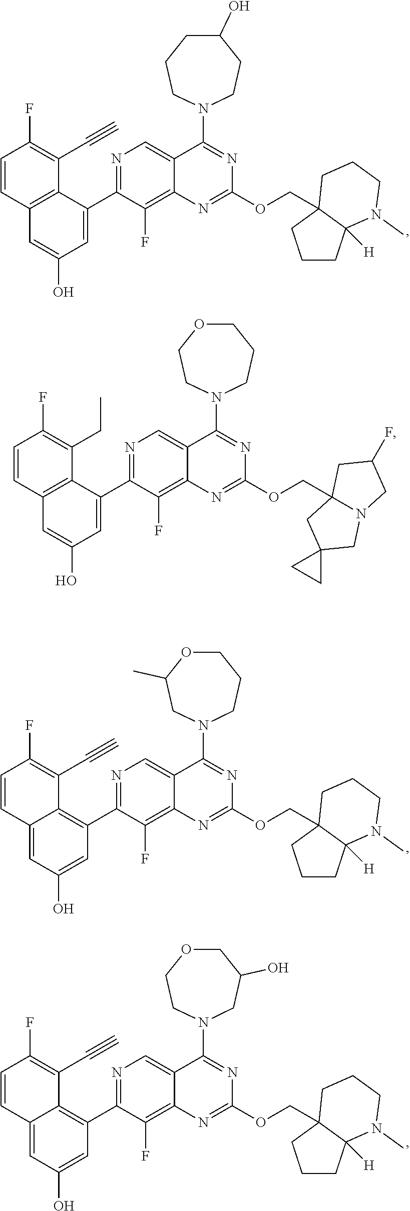 [SMILES: C#Cc1c(F)ccc2cc(O)cc(-c3ncc4c(N5CCCC(O)CC5)nc(OCC56CCCC5N(C)CCC6)nc4c3F)c12 ; CCc1c(F)ccc2cc(O)cc(-c3ncc4c(N5CCCOCC5)nc(OCC56CC(F)CN5CC5(CC5)C6)nc4c3F)c12 ; C#Cc1c(F)ccc2cc(O)cc(-c3ncc4c(N5CCCOC(C)C5)nc(OCC56CCCC5N(C)CCC6)nc4c3F)c12 ; C#Cc1c(F)ccc2cc(O)cc(-c3ncc4c(N5CCOCC(O)C5)nc(OCC56CCCC5N(C)CCC6)nc4c3F)c12]

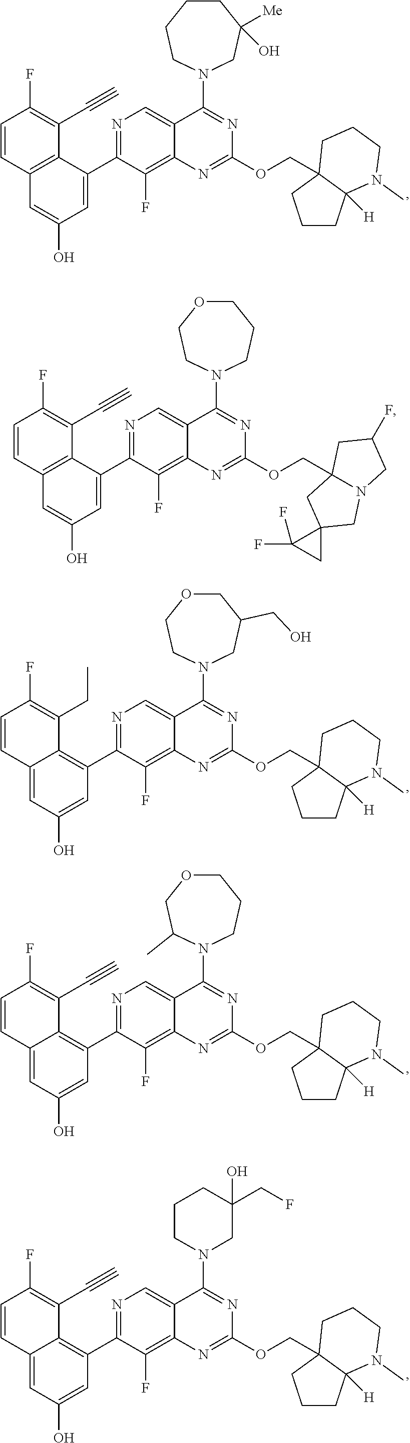 [SMILES: C#Cc1c(F)ccc2cc(O)cc(-c3ncc4c(N5CCCCC(C)(O)C5)nc(OCC56CCCC5N(C)CCC6)nc4c3F)c12 ; C#Cc1c(F)ccc2cc(O)cc(-c3ncc4c(N5CCCOCC5)nc(OCC56CC(F)CN5CC5(C6)CC5(F)F)nc4c3F)c12 ; CCc1c(F)ccc2cc(O)cc(-c3ncc4c(N5CCOCC(CO)C5)nc(OCC56CCCC5N(C)CCC6)nc4c3F)c12 ; C#Cc1c(F)ccc2cc(O)cc(-c3ncc4c(N5CCCOCC5C)nc(OCC56CCCC5N(C)CCC6)nc4c3F)c12 ; C#Cc1c(F)ccc2cc(O)cc(-c3ncc4c(N5CCCC(O)(CF)C5)nc(OCC56CCCC5N(C)CCC6)nc4c3F)c12]

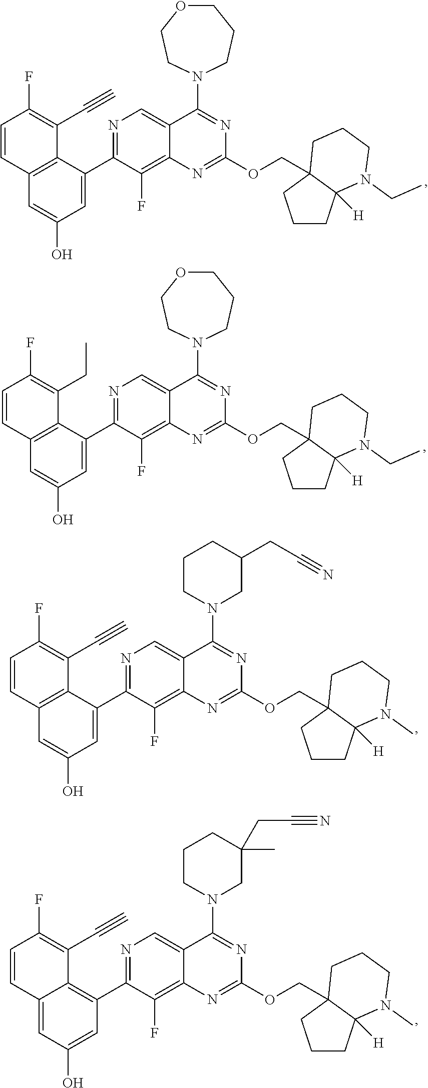 [SMILES: C#Cc1c(F)ccc2cc(O)cc(-c3ncc4c(N5CCCOCC5)nc(OCC56CCCC5N(CC)CCC6)nc4c3F)c12 ; CCc1c(F)ccc2cc(O)cc(-c3ncc4c(N5CCCOCC5)nc(OCC56CCCC5N(CC)CCC6)nc4c3F)c12 ; C#Cc1c(F)ccc2cc(O)cc(-c3ncc4c(N5CCCC(CC#N)C5)nc(OCC56CCCC5N(C)CCC6)nc4c3F)c12 ; C#Cc1c(F)ccc2cc(O)cc(-c3ncc4c(N5CCCC(C)(CC#N)C5)nc(OCC56CCCC5N(C)CCC6)nc4c3F)c12]

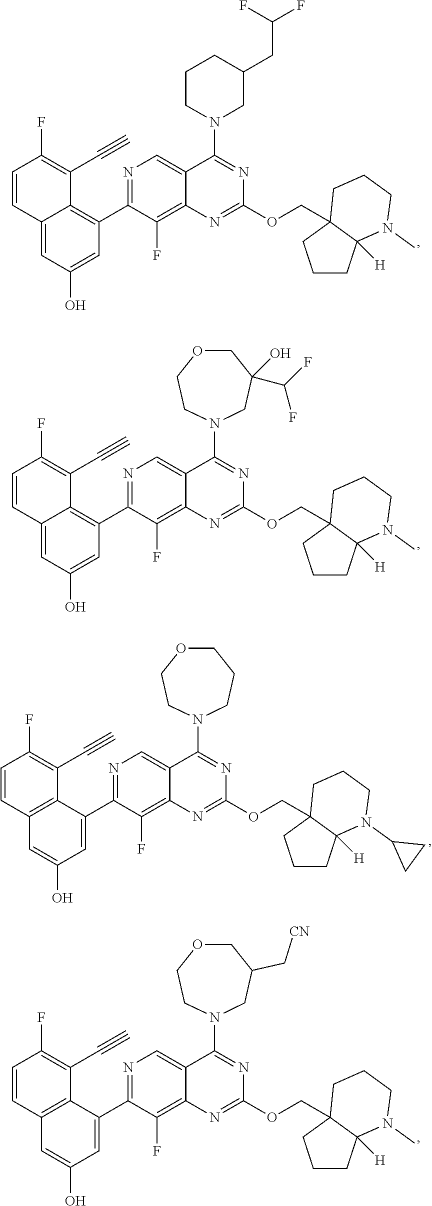 [SMILES: C#Cc1c(F)ccc2cc(O)cc(-c3ncc4c(N5CCCC(CC(F)F)C5)nc(OCC56CCCC5N(C)CCC6)nc4c3F)c12 ; C#Cc1c(F)ccc2cc(O)cc(-c3ncc4c(N5CCOCC(O)(C(F)F)C5)nc(OCC56CCCC5N(C)CCC6)nc4c3F)c12 ; C#Cc1c(F)ccc2cc(O)cc(-c3ncc4c(N5CCCOCC5)nc(OCC56CCCC5N(C5CC5)CCC6)nc4c3F)c12 ; C#Cc1c(F)ccc2cc(O)cc(-c3ncc4c(N5CCOCC(CC#N)C5)nc(OCC56CCCC5N(C)CCC6)nc4c3F)c12]

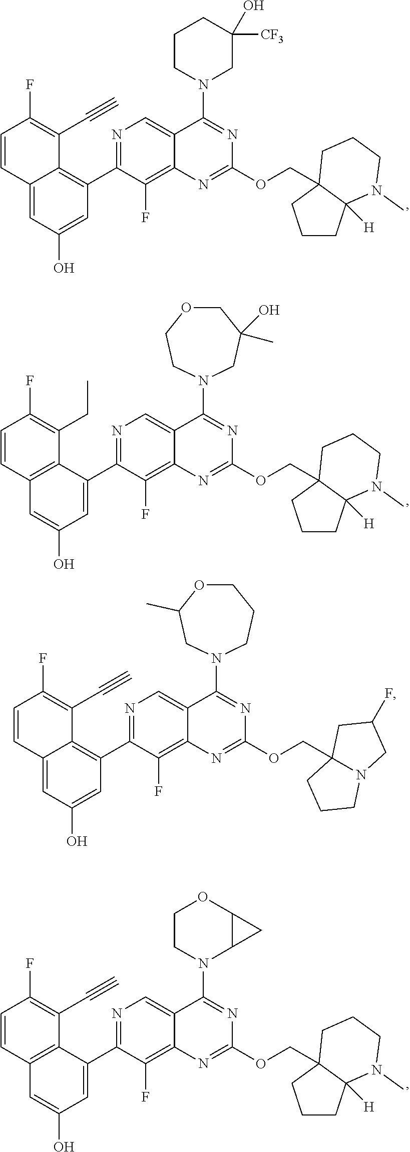 [SMILES: C#Cc1c(F)ccc2cc(O)cc(-c3ncc4c(N5CCCC(O)(C(F)(F)F)C5)nc(OCC56CCCC5N(C)CCC6)nc4c3F)c12 ; CCc1c(F)ccc2cc(O)cc(-c3ncc4c(N5CCOCC(C)(O)C5)nc(OCC56CCCC5N(C)CCC6)nc4c3F)c12 ; C#Cc1c(F)ccc2cc(O)cc(-c3ncc4c(N5CCCOC(C)C5)nc(OCC56CCCN5CC(F)C6)nc4c3F)c12 ; C#Cc1c(F)ccc2cc(O)cc(-c3ncc4c(N5CCOC6CC65)nc(OCC56CCCC5N(C)CCC6)nc4c3F)c12]

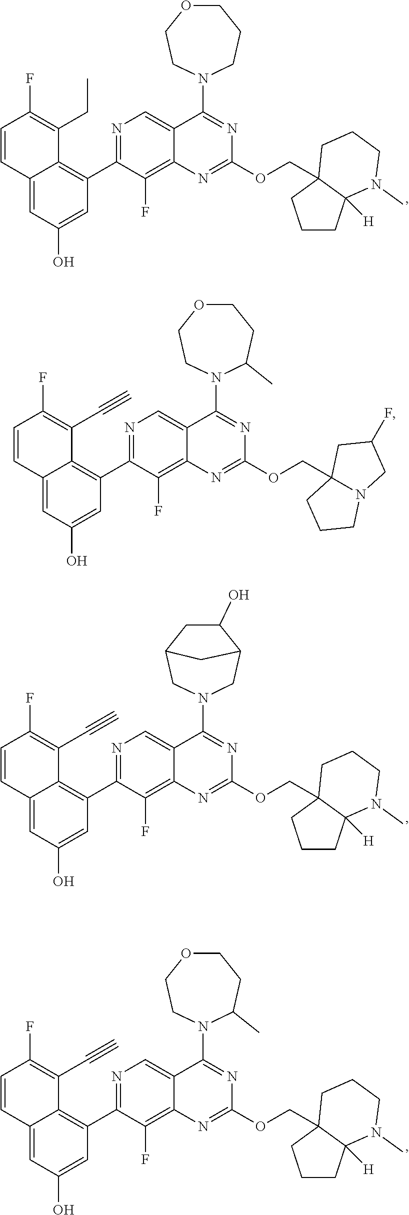 [SMILES: CCc1c(F)ccc2cc(O)cc(-c3ncc4c(N5CCCOCC5)nc(OCC56CCCC5N(C)CCC6)nc4c3F)c12 ; C#Cc1c(F)ccc2cc(O)cc(-c3ncc4c(N5CCOCCC5C)nc(OCC56CCCN5CC(F)C6)nc4c3F)c12 ; C#Cc1c(F)ccc2cc(O)cc(-c3ncc4c(N5CC6CC(O)C(C6)C5)nc(OCC56CCCC5N(C)CCC6)nc4c3F)c12 ; C#Cc1c(F)ccc2cc(O)cc(-c3ncc4c(N5CCOCCC5C)nc(OCC56CCCC5N(C)CCC6)nc4c3F)c12]

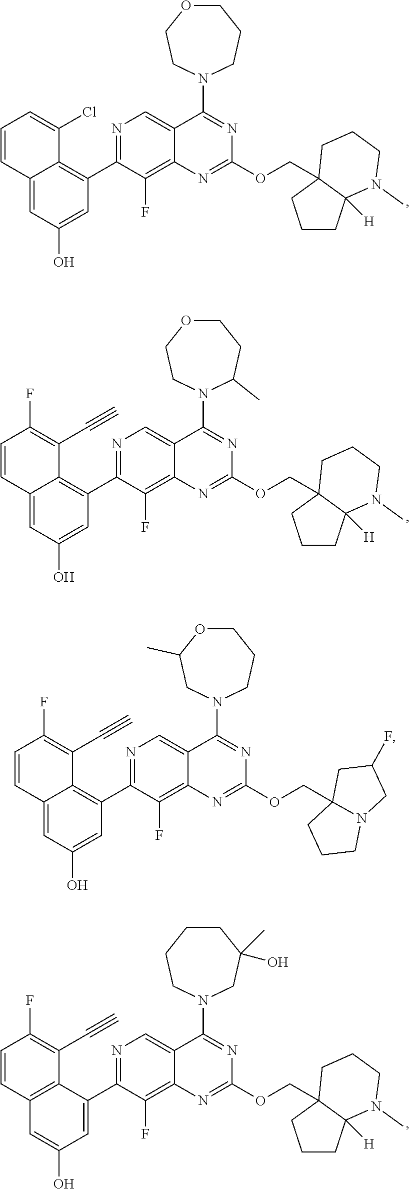 [SMILES: CN1CCCC2(COc3nc(N4CCCOCC4)c4cnc(-c5cc(O)cc6cccc(Cl)c56)c(F)c4n3)CCCC12 ; C#Cc1c(F)ccc2cc(O)cc(-c3ncc4c(N5CCOCCC5C)nc(OCC56CCCC5N(C)CCC6)nc4c3F)c12 ; C#Cc1c(F)ccc2cc(O)cc(-c3ncc4c(N5CCCOC(C)C5)nc(OCC56CCCN5CC(F)C6)nc4c3F)c12 ; C#Cc1c(F)ccc2cc(O)cc(-c3ncc4c(N5CCCCC(C)(O)C5)nc(OCC56CCCC5N(C)CCC6)nc4c3F)c12]

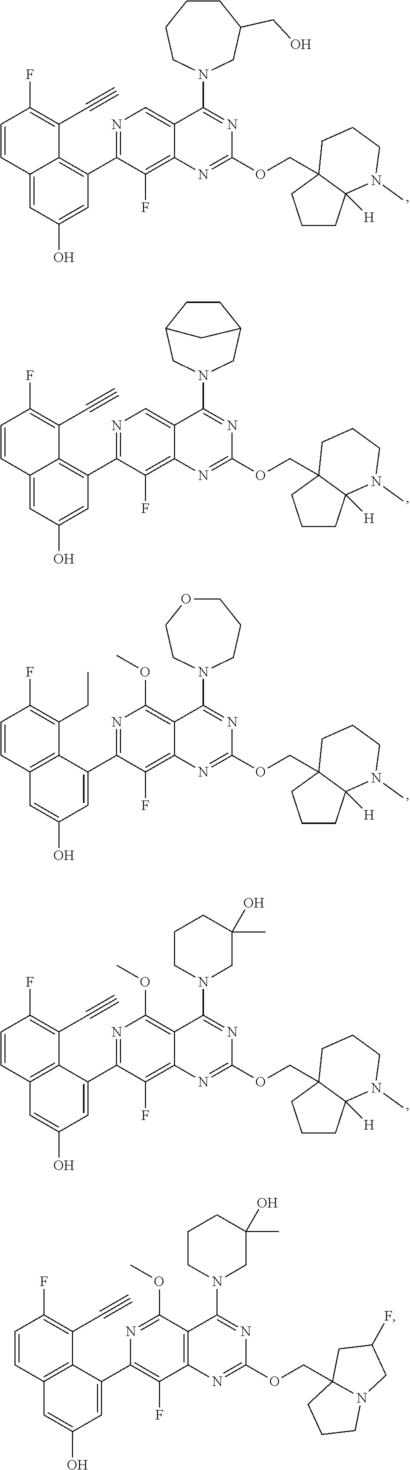 [SMILES: C#Cc1c(F)ccc2cc(O)cc(-c3ncc4c(N5CCCCC(CO)C5)nc(OCC56CCCC5N(C)CCC6)nc4c3F)c12 ; C#Cc1c(F)ccc2cc(O)cc(-c3ncc4c(N5CC6CCC(C6)C5)nc(OCC56CCCC5N(C)CCC6)nc4c3F)c12 ; CCc1c(F)ccc2cc(O)cc(-c3nc(OC)c4c(N5CCCOCC5)nc(OCC56CCCC5N(C)CCC6)nc4c3F)c12 ; C#Cc1c(F)ccc2cc(O)cc(-c3nc(OC)c4c(N5CCCC(C)(O)C5)nc(OCC56CCCC5N(C)CCC6)nc4c3F)c12 ; C#Cc1c(F)ccc2cc(O)cc(-c3nc(OC)c4c(N5CCCC(C)(O)C5)nc(OCC56CCCN5CC(F)C6)nc4c3F)c12]

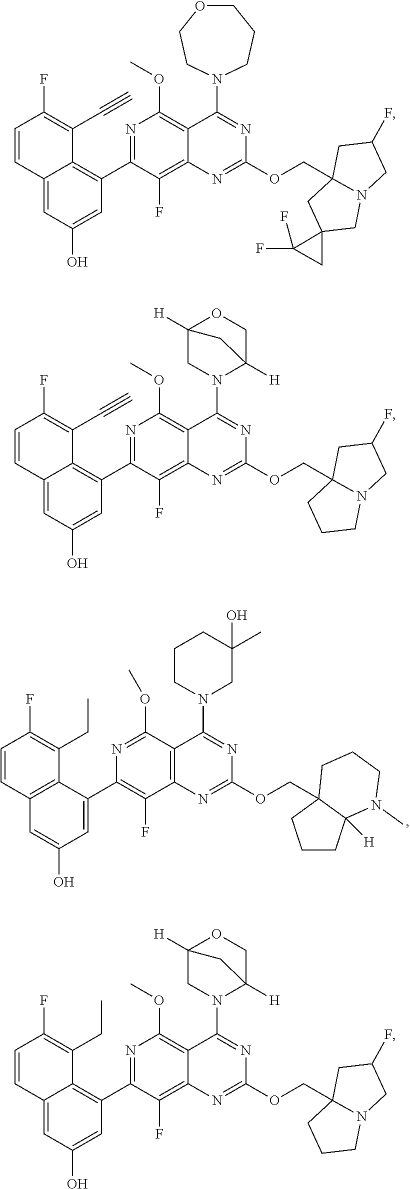 [SMILES: C#Cc1c(F)ccc2cc(O)cc(-c3nc(OC)c4c(N5CCCOCC5)nc(OCC56CC(F)CN5CC5(C6)CC5(F)F)nc4c3F)c12 ; C#Cc1c(F)ccc2cc(O)cc(-c3nc(OC)c4c(N5CC6CC5CO6)nc(OCC56CCCN5CC(F)C6)nc4c3F)c12 ; CCc1c(F)ccc2cc(O)cc(-c3nc(OC)c4c(N5CCCC(C)(O)C5)nc(OCC56CCCC5N(C)CCC6)nc4c3F)c12 ; CCc1c(F)ccc2cc(O)cc(-c3nc(OC)c4c(N5CC6CC5CO6)nc(OCC56CCCN5CC(F)C6)nc4c3F)c12]

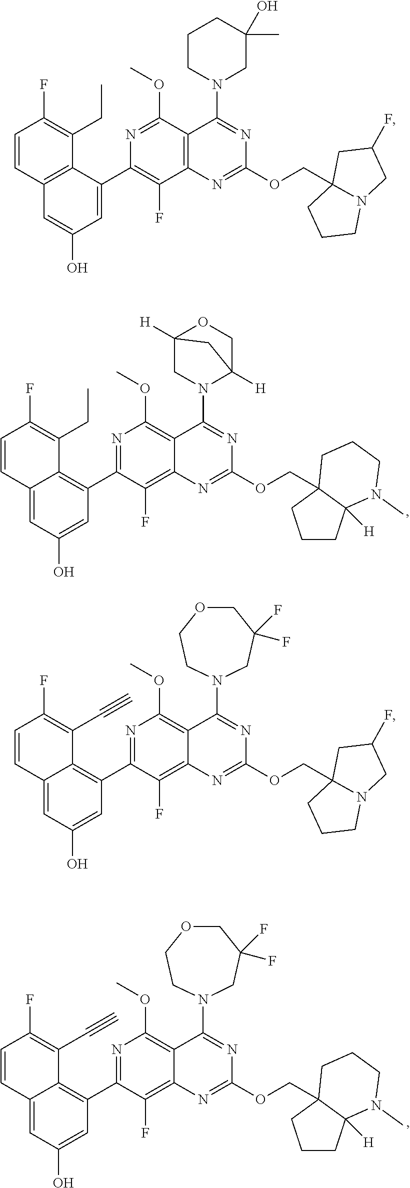 [SMILES: CCc1c(F)ccc2cc(O)cc(-c3nc(OC)c4c(N5CCCC(C)(O)C5)nc(OCC56CCCN5CC(F)C6)nc4c3F)c12 ; CCc1c(F)ccc2cc(O)cc(-c3nc(OC)c4c(N5CC6CC5CO6)nc(OCC56CCCC5N(C)CCC6)nc4c3F)c12 ; C#Cc1c(F)ccc2cc(O)cc(-c3nc(OC)c4c(N5CCOCC(F)(F)C5)nc(OCC56CCCN5CC(F)C6)nc4c3F)c12 ; C#Cc1c(F)ccc2cc(O)cc(-c3nc(OC)c4c(N5CCOCC(F)(F)C5)nc(OCC56CCCC5N(C)CCC6)nc4c3F)c12]

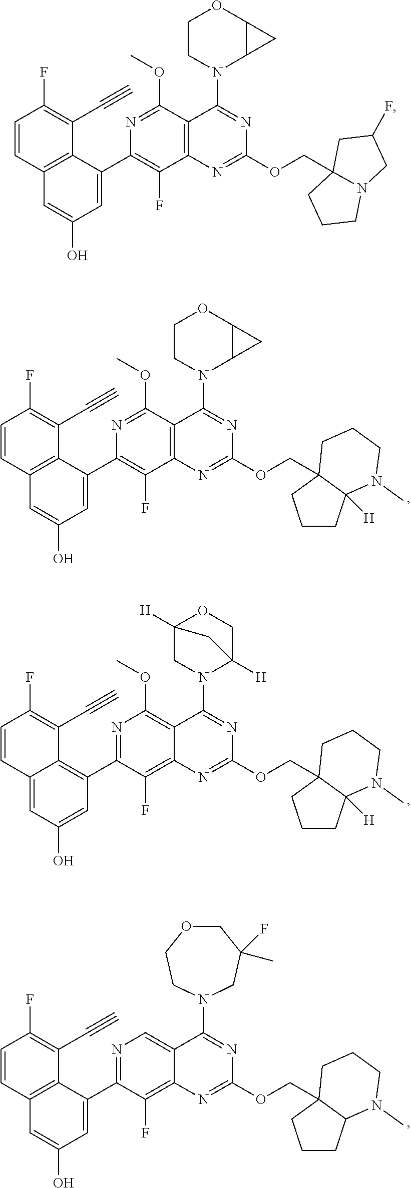 [SMILES: C#Cc1c(F)ccc2cc(O)cc(-c3nc(OC)c4c(N5CCOC6CC65)nc(OCC56CCCN5CC(F)C6)nc4c3F)c12 ; C#Cc1c(F)ccc2cc(O)cc(-c3nc(OC)c4c(N5CCOC6CC65)nc(OCC56CCCC5N(C)CCC6)nc4c3F)c12 ; C#Cc1c(F)ccc2cc(O)cc(-c3nc(OC)c4c(N5CC6CC5CO6)nc(OCC56CCCC5N(C)CCC6)nc4c3F)c12 ; C#Cc1c(F)ccc2cc(O)cc(-c3ncc4c(N5CCOCC(C)(F)C5)nc(OCC56CCCC5N(C)CCC6)nc4c3F)c12]

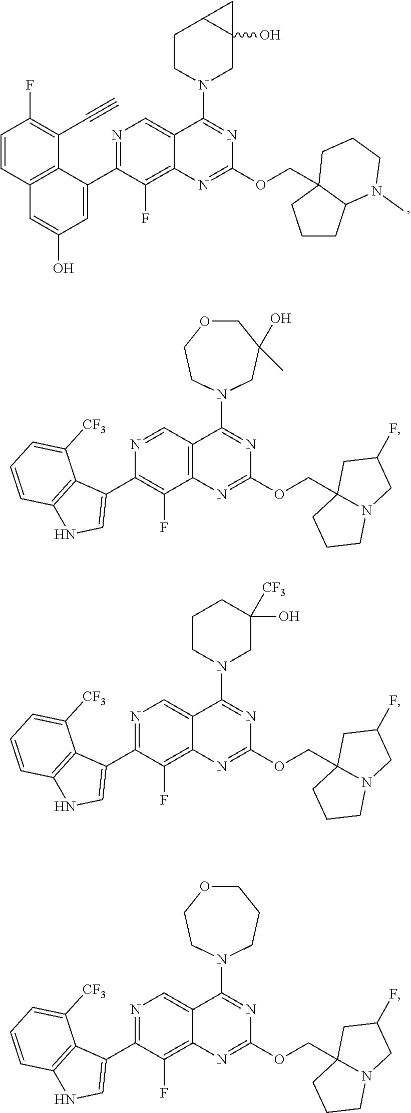 [SMILES: C#Cc1c(F)ccc2cc(O)cc(-c3ncc4c(N5CCC6CC6(O)C5)nc(OCC56CCCC5N(C)CCC6)nc4c3F)c12 ; CC1(O)COCCN(c2nc(OCC34CCCN3CC(F)C4)nc3c(F)c(-c4c[nH]c5cccc(C(F)(F)F)c45)ncc23)C1 ; OC1(C(F)(F)F)CCCN(c2nc(OCC34CCCN3CC(F)C4)nc3c(F)c(-c4c[nH]c5cccc(C(F)(F)F)c45)ncc23)C1 ; Fc1c(-c2c[nH]c3cccc(C(F)(F)F)c23)ncc2c(N3CCCOCC3)nc(OCC34CCCN3CC(F)C4)nc12]

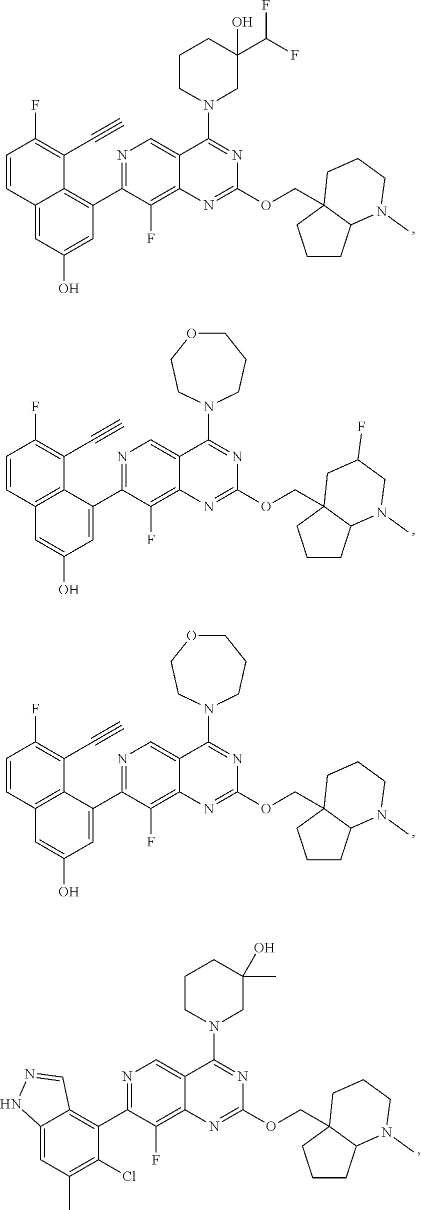 [SMILES: C#Cc1c(F)ccc2cc(O)cc(-c3ncc4c(N5CCCC(O)(C(F)F)C5)nc(OCC56CCCC5N(C)CCC6)nc4c3F)c12 ; C#Cc1c(F)ccc2cc(O)cc(-c3ncc4c(N5CCCOCC5)nc(OCC56CCCC5N(C)CC(F)C6)nc4c3F)c12 ; C#Cc1c(F)ccc2cc(O)cc(-c3ncc4c(N5CCCOCC5)nc(OCC56CCCC5N(C)CCC6)nc4c3F)c12 ; Cc1cc2[nH]ncc2c(-c2ncc3c(N4CCCC(C)(O)C4)nc(OCC45CCCC4N(C)CCC5)nc3c2F)c1Cl]

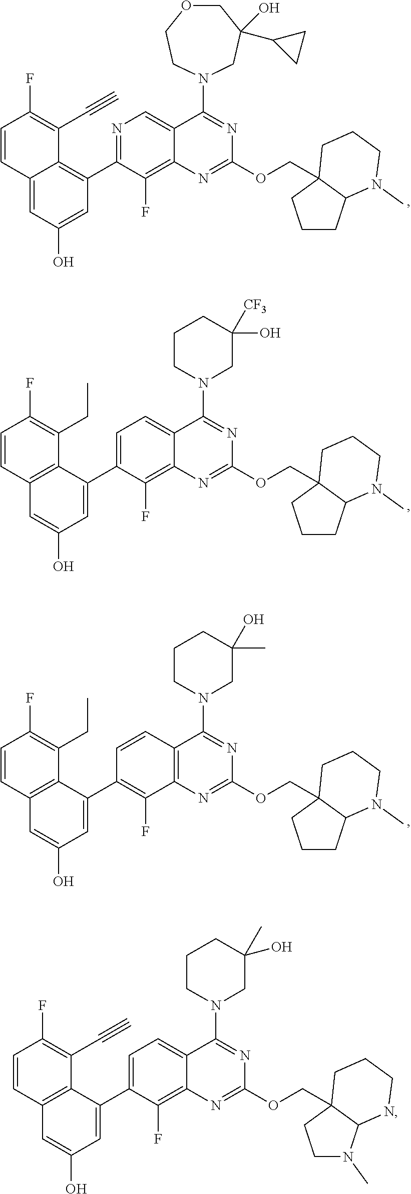

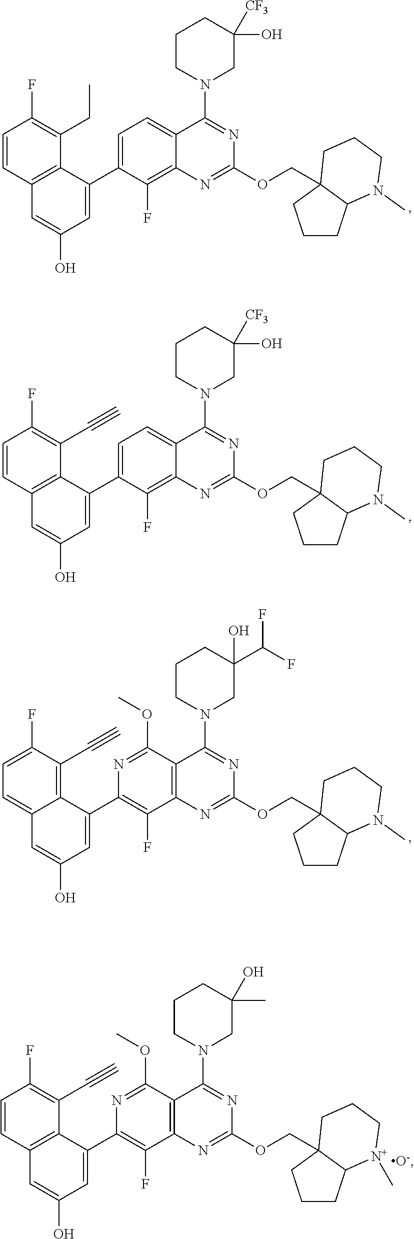 [SMILES: CCc1c(F)ccc2cc(O)cc(-c3ccc4c(N5CCCC(O)(C(F)(F)F)C5)nc(OCC56CCCC5N(C)CCC6)nc4c3F)c12 ; C#Cc1c(F)ccc2cc(O)cc(-c3ccc4c(N5CCCC(O)(C(F)(F)F)C5)nc(OCC56CCCC5N(C)CCC6)nc4c3F)c12 ; C#Cc1c(F)ccc2cc(O)cc(-c3nc(OC)c4c(N5CCCC(O)(C(F)F)C5)nc(OCC56CCCC5N(C)CCC6)nc4c3F)c12 ; C#Cc1c(F)ccc2cc(O)cc(-c3nc(OC)c4c(N5CCCC(C)(O)C5)nc(OCC56CCCC5[NH+](C)CCC6)nc4c3F)c12]

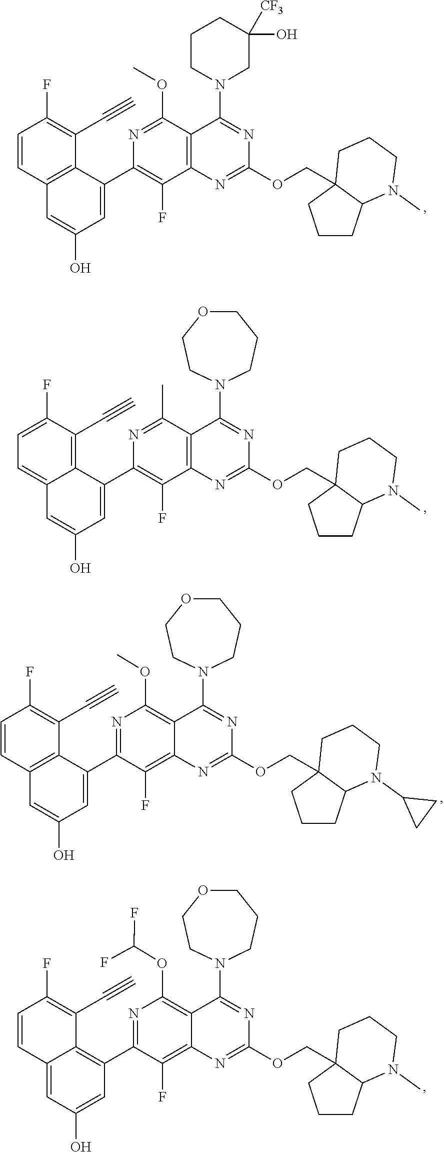 [SMILES: C#Cc1c(F)ccc2cc(O)cc(-c3nc(OC)c4c(N5CCCC(O)(C(F)(F)F)C5)nc(OCC56CCCC5N(C)CCC6)nc4c3F)c12 ; C#Cc1c(F)ccc2cc(O)cc(-c3nc(C)c4c(N5CCCOCC5)nc(OCC56CCCC5N(C)CCC6)nc4c3F)c12 ; C#Cc1c(F)ccc2cc(O)cc(-c3nc(OC)c4c(N5CCCOCC5)nc(OCC56CCCC5N(C5CC5)CCC6)nc4c3F)c12 ; C#Cc1c(F)ccc2cc(O)cc(-c3nc(OC(F)F)c4c(N5CCCOCC5)nc(OCC56CCCC5N(C)CCC6)nc4c3F)c12]

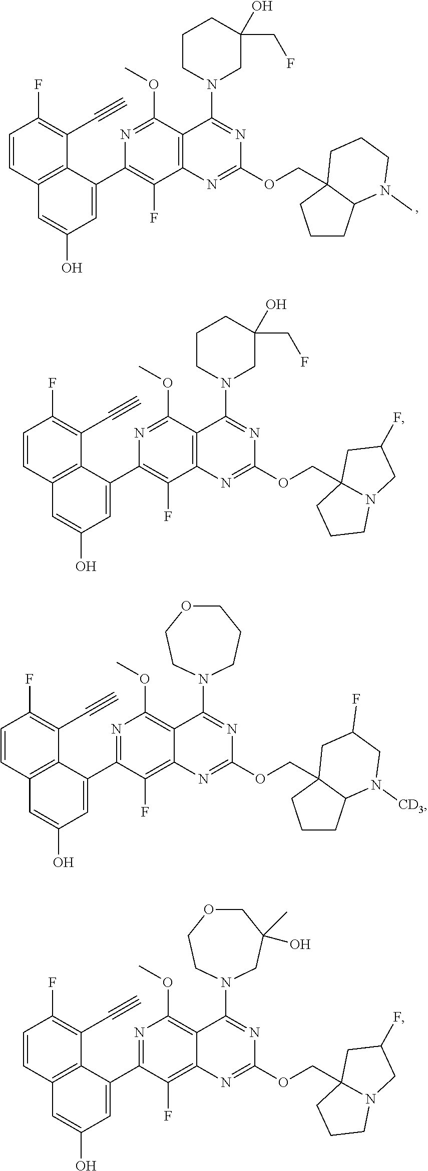 [SMILES: C#Cc1c(F)ccc2cc(O)cc(-c3nc(OC)c4c(N5CCCC(O)(CF)C5)nc(OCC56CCCC5N(C)CCC6)nc4c3F)c12 ; C#Cc1c(F)ccc2cc(O)cc(-c3nc(OC)c4c(N5CCCC(O)(CF)C5)nc(OCC56CCCN5CC(F)C6)nc4c3F)c12 ; [2H]C([2H])([2H])N1CC(F)CC2(COc3nc(N4CCCOCC4)c4c(OC)nc(-c5cc(O)cc6ccc(F)c(C#C)c56)c(F)c4n3)CCCC12 ; C#Cc1c(F)ccc2cc(O)cc(-c3nc(OC)c4c(N5CCOCC(C)(O)C5)nc(OCC56CCCN5CC(F)C6)nc4c3F)c12]

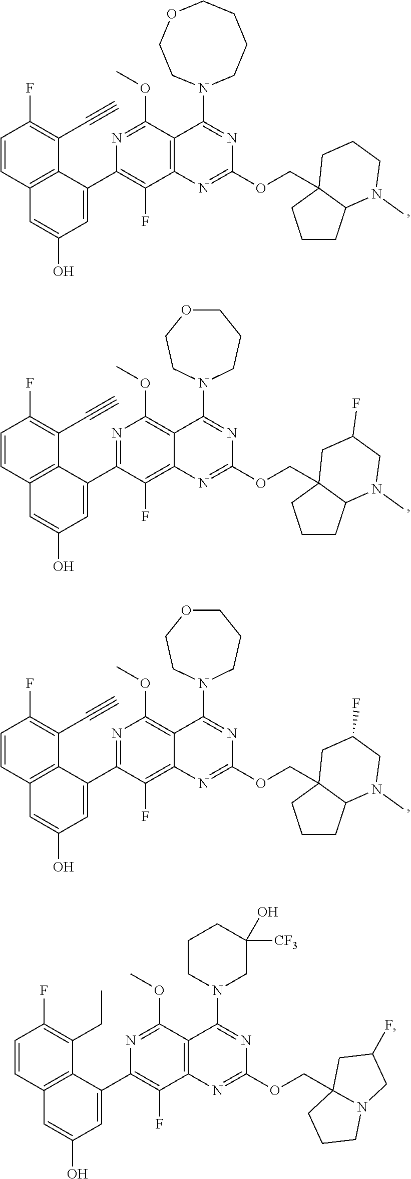 [SMILES: C#Cc1c(F)ccc2cc(O)cc(-c3nc(OC)c4c(N5CCCCOCC5)nc(OCC56CCCC5N(C)CCC6)nc4c3F)c12 ; C#Cc1c(F)ccc2cc(O)cc(-c3nc(OC)c4c(N5CCCOCC5)nc(OCC56CCCC5N(C)CC(F)C6)nc4c3F)c12 ; C#Cc1c(F)ccc2cc(O)cc(-c3nc(OC)c4c(N5CCCOCC5)nc(OCC56CCCC5N(C)C[C@@H](F)C6)nc4c3F)c12 ; CCc1c(F)ccc2cc(O)cc(-c3nc(OC)c4c(N5CCCC(O)(C(F)(F)F)C5)nc(OCC56CCCN5CC(F)C6)nc4c3F)c12]

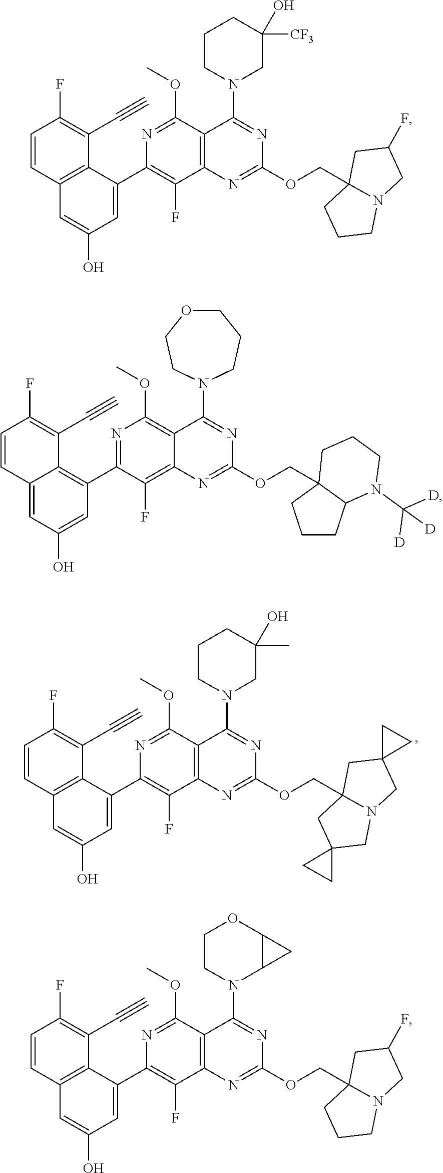

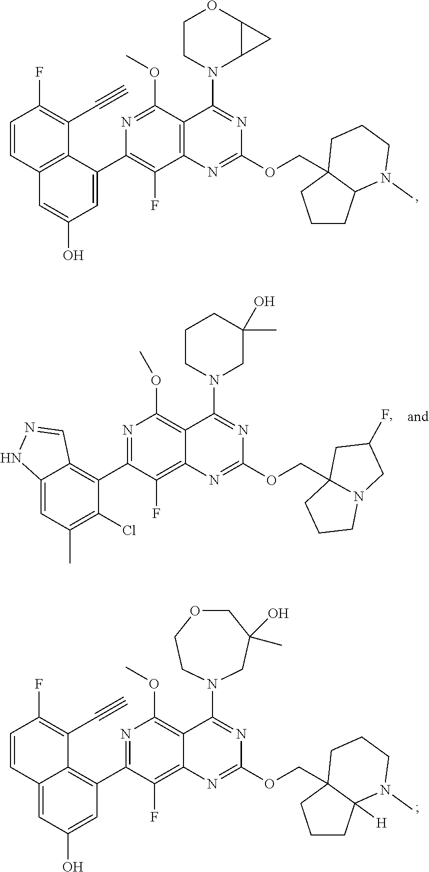 [SMILES: C#Cc1c(F)ccc2cc(O)cc(-c3nc(OC)c4c(N5CCOC6CC65)nc(OCC56CCCC5N(C)CCC6)nc4c3F)c12 ; COc1nc(-c2c(Cl)c(C)cc3[nH]ncc23)c(F)c2nc(OCC34CCCN3CC(F)C4)nc(N3CCCC(C)(O)C3)c12 ; C#Cc1c(F)ccc2cc(O)cc(-c3nc(OC)c4c(N5CCOCC(C)(O)C5)nc(OCC56CCCC5N(C)CCC6)nc4c3F)c12]

or a pharmaceutically acceptable salt thereof.

In some aspects, the present disclosure provides a compound selected from the group consisting of:

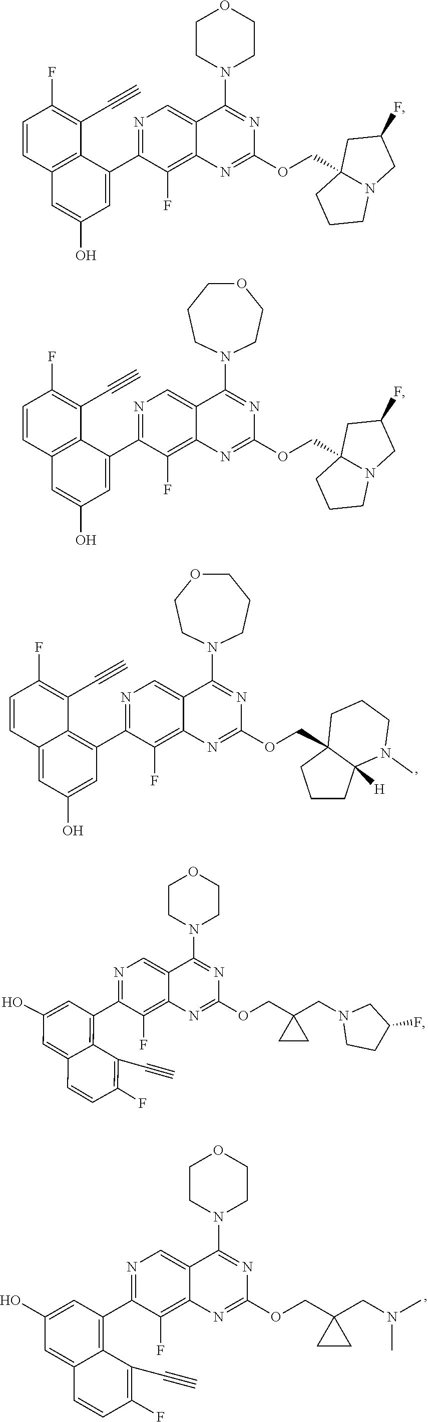 [SMILES: C#Cc1c(F)ccc2cc(O)cc(-c3ncc4c(N5CCOCC5)nc(OC[C@@]56CCCN5C[C@H](F)C6)nc4c3F)c12 ; C#Cc1c(F)ccc2cc(O)cc(-c3ncc4c(N5CCCOCC5)nc(OC[C@@]56CCCN5C[C@H](F)C6)nc4c3F)c12 ; C#Cc1c(F)ccc2cc(O)cc(-c3ncc4c(N5CCCOCC5)nc(OC[C@]56CCC[C@H]5N(C)CCC6)nc4c3F)c12 ; C#Cc1c(F)ccc2cc(O)cc(-c3ncc4c(N5CCOCC5)nc(OCC5(CN6CC[C@@H](F)C6)CC5)nc4c3F)c12 ; C#Cc1c(F)ccc2cc(O)cc(-c3ncc4c(N5CCOCC5)nc(OCC5(CN(C)C)CC5)nc4c3F)c12]

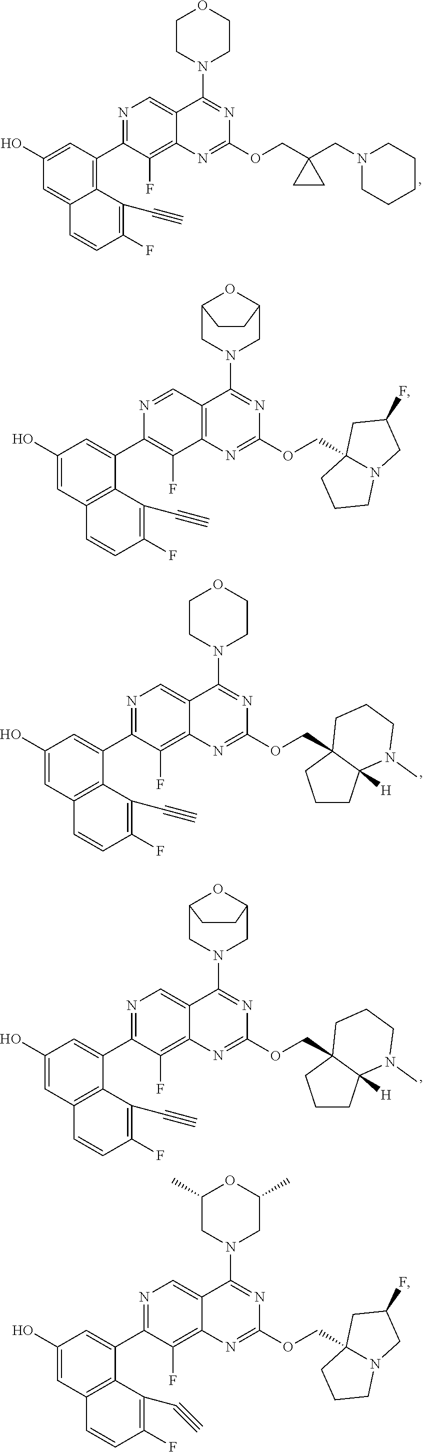

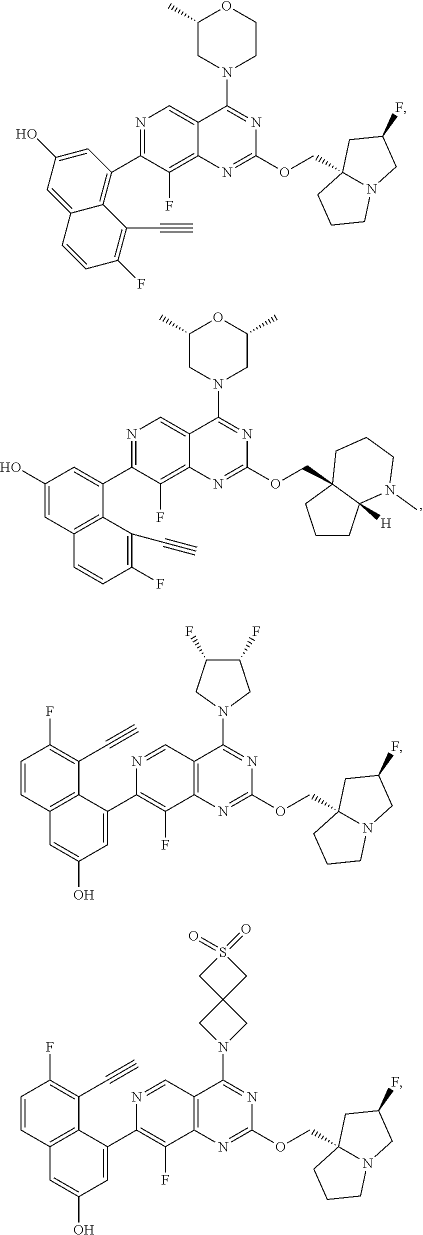

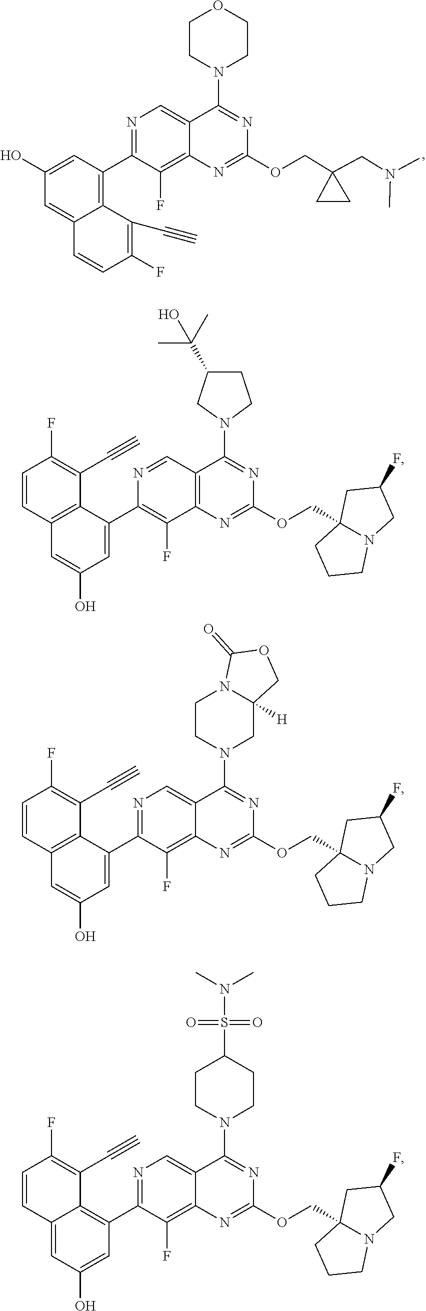

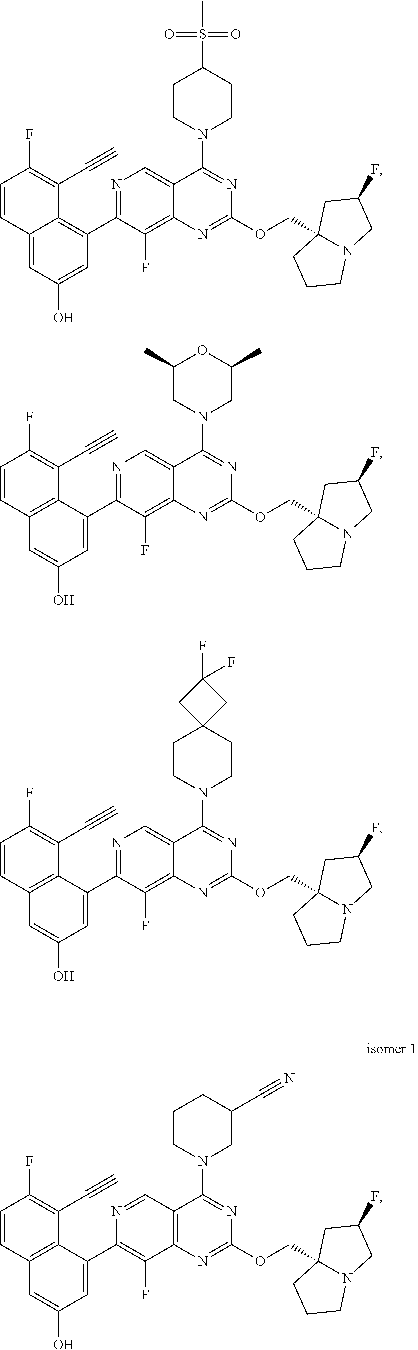 [SMILES: C#Cc1c(F)ccc2cc(O)cc(-c3ncc4c(N5CCC(S(C)(=O)=O)CC5)nc(OC[C@@]56CCCN5C[C@H](F)C6)nc4c3F)c12 ; C#Cc1c(F)ccc2cc(O)cc(-c3ncc4c(N5C[C@@H](C)O[C@@H](C)C5)nc(OC[C@@]56CCCN5C[C@H](F)C6)nc4c3F)c12 ; C#Cc1c(F)ccc2cc(O)cc(-c3ncc4c(N5CCC6(CC5)CC(F)(F)C6)nc(OC[C@@]56CCCN5C[C@H](F)C6)nc4c3F)c12 ; C#Cc1c(F)ccc2cc(O)cc(-c3ncc4c(N5CCCC(C#N)C5)nc(OC[C@@]56CCCN5C[C@H](F)C6)nc4c3F)c12]

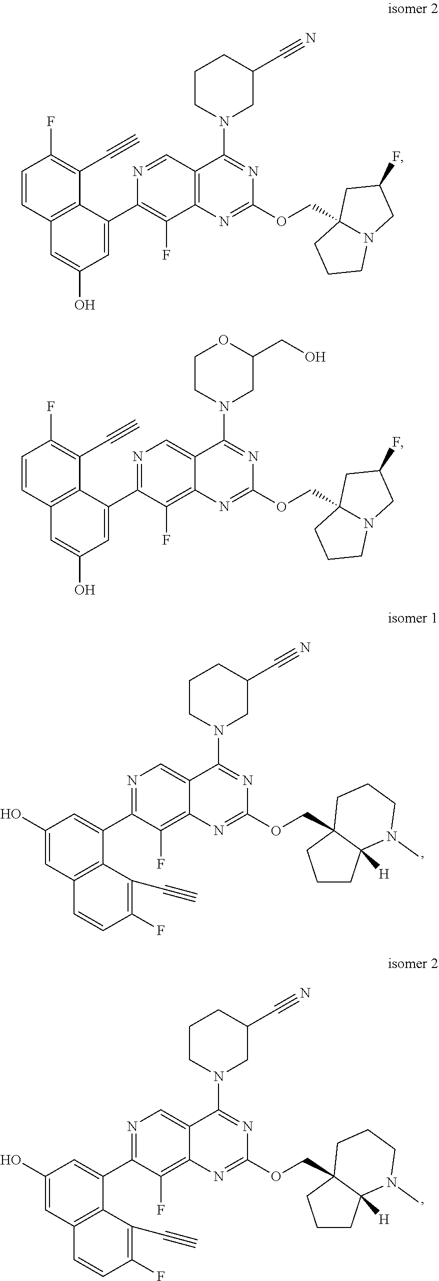 [SMILES: C#Cc1c(F)ccc2cc(O)cc(-c3ncc4c(N5CCCC(C#N)C5)nc(OC[C@@]56CCCN5C[C@H](F)C6)nc4c3F)c12 ; C#Cc1c(F)ccc2cc(O)cc(-c3ncc4c(N5CCOC(CO)C5)nc(OC[C@@]56CCCN5C[C@H](F)C6)nc4c3F)c12 ; C#Cc1c(F)ccc2cc(O)cc(-c3ncc4c(N5CCCC(C#N)C5)nc(OC[C@]56CCC[C@H]5N(C)CCC6)nc4c3F)c12 ; C#Cc1c(F)ccc2cc(O)cc(-c3ncc4c(N5CCCC(C#N)C5)nc(OC[C@]56CCC[C@H]5N(C)CCC6)nc4c3F)c12]

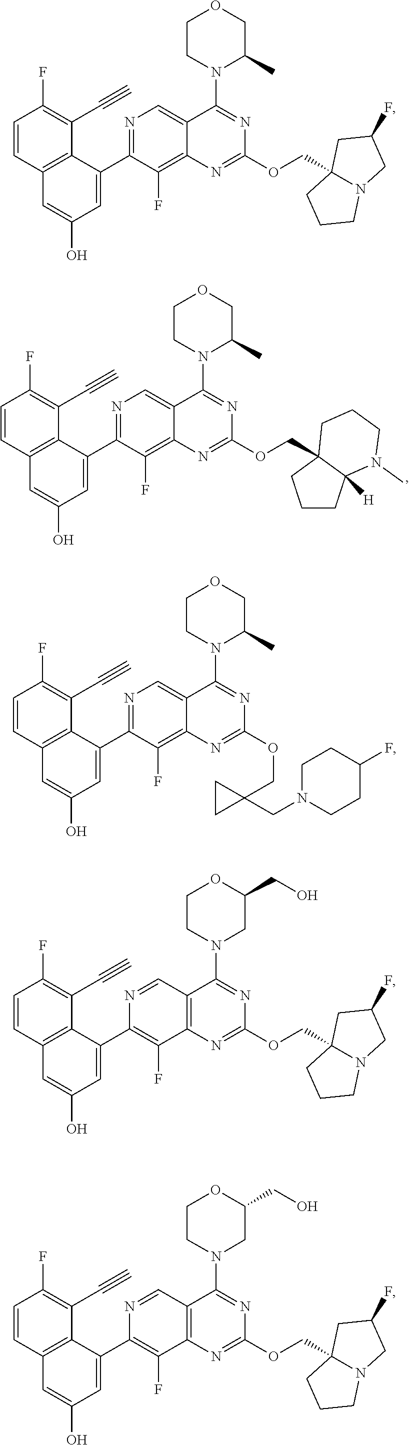 [SMILES: C#Cc1c(F)ccc2cc(O)cc(-c3ncc4c(N5CCOC[C@H]5C)nc(OC[C@@]56CCCN5C[C@H](F)C6)nc4c3F)c12 ; C#Cc1c(F)ccc2cc(O)cc(-c3ncc4c(N5CCOC[C@H]5C)nc(OC[C@]56CCC[C@H]5N(C)CCC6)nc4c3F)c12 ; C#Cc1c(F)ccc2cc(O)cc(-c3ncc4c(N5CCOC[C@H]5C)nc(OCC5(CN6CCC(F)CC6)CC5)nc4c3F)c12 ; C#Cc1c(F)ccc2cc(O)cc(-c3ncc4c(N5CCO[C@@H](CO)C5)nc(OC[C@@]56CCCN5C[C@H](F)C6)nc4c3F)c12 ; C#Cc1c(F)ccc2cc(O)cc(-c3ncc4c(N5CCO[C@H](CO)C5)nc(OC[C@@]56CCCN5C[C@H](F)C6)nc4c3F)c12]

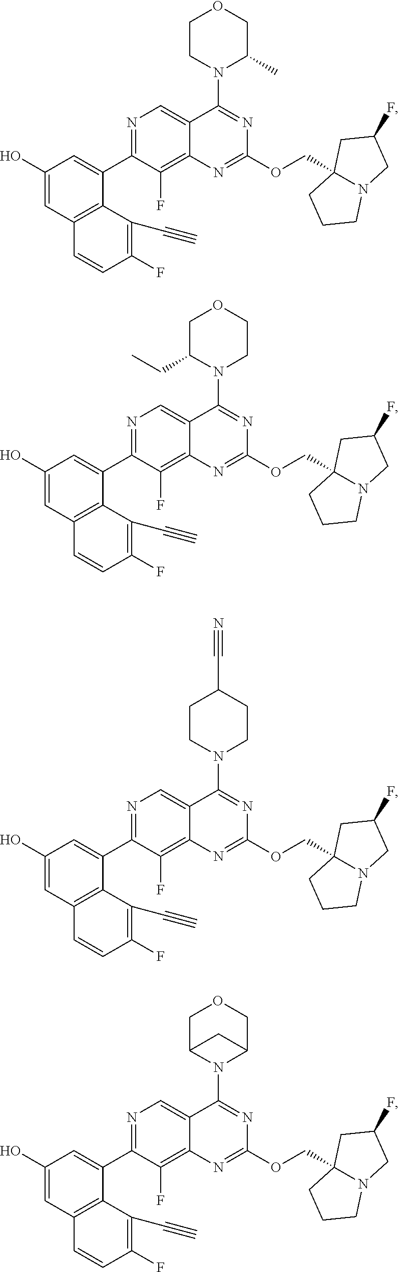 [SMILES: C#Cc1c(F)ccc2cc(O)cc(-c3ncc4c(N5CCOC[C@@H]5C)nc(OC[C@@]56CCCN5C[C@H](F)C6)nc4c3F)c12 ; C#Cc1c(F)ccc2cc(O)cc(-c3ncc4c(N5CCOC[C@H]5CC)nc(OC[C@@]56CCCN5C[C@H](F)C6)nc4c3F)c12 ; C#Cc1c(F)ccc2cc(O)cc(-c3ncc4c(N5CCC(C#N)CC5)nc(OC[C@@]56CCCN5C[C@H](F)C6)nc4c3F)c12 ; C#Cc1c(F)ccc2cc(O)cc(-c3ncc4c(N5C6COCC5C6)nc(OC[C@@]56CCCN5C[C@H](F)C6)nc4c3F)c12]

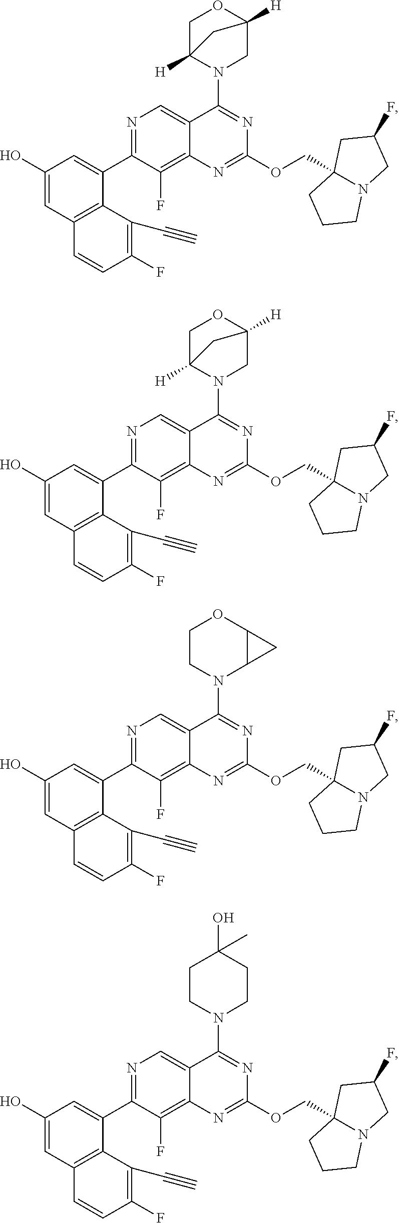 [SMILES: C#Cc1c(F)ccc2cc(O)cc(-c3ncc4c(N5C[C@@H]6C[C@H]5CO6)nc(OC[C@@]56CCCN5C[C@H](F)C6)nc4c3F)c12 ; C#Cc1c(F)ccc2cc(O)cc(-c3ncc4c(N5C[C@H]6C[C@@H]5CO6)nc(OC[C@@]56CCCN5C[C@H](F)C6)nc4c3F)c12 ; C#Cc1c(F)ccc2cc(O)cc(-c3ncc4c(N5CCOC6CC65)nc(OC[C@@]56CCCN5C[C@H](F)C6)nc4c3F)c12 ; C#Cc1c(F)ccc2cc(O)cc(-c3ncc4c(N5CCC(C)(O)CC5)nc(OC[C@@]56CCCN5C[C@H](F)C6)nc4c3F)c12]

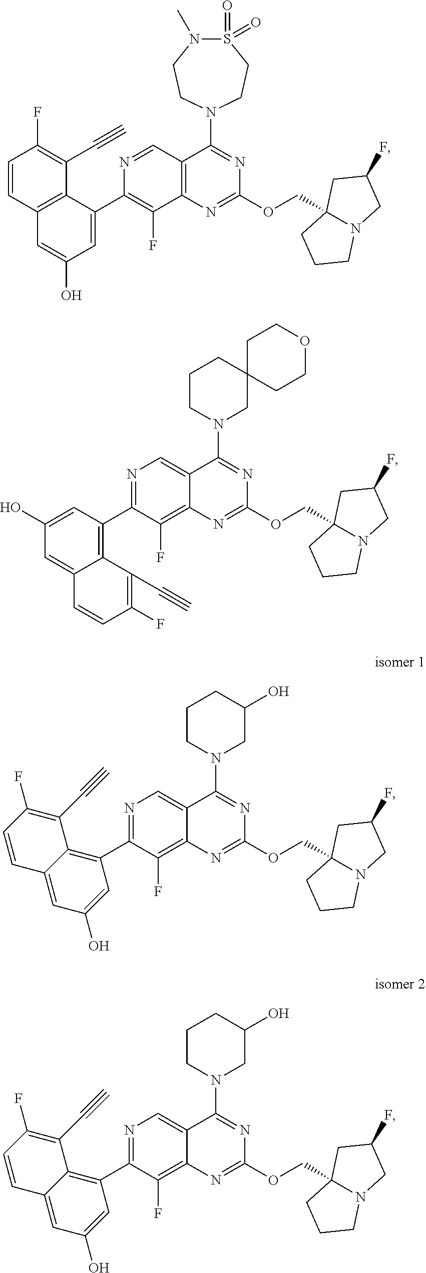 [SMILES: C#Cc1c(F)ccc2cc(O)cc(-c3ncc4c(N5CCN(C)S(=O)(=O)CC5)nc(OC[C@@]56CCCN5C[C@H](F)C6)nc4c3F)c12 ; C#Cc1c(F)ccc2cc(O)cc(-c3ncc4c(N5CCCC6(CCOCC6)C5)nc(OC[C@@]56CCCN5C[C@H](F)C6)nc4c3F)c12 ; C#Cc1c(F)ccc2cc(O)cc(-c3ncc4c(N5CCCC(O)C5)nc(OC[C@@]56CCCN5C[C@H](F)C6)nc4c3F)c12 ; C#Cc1c(F)ccc2cc(O)cc(-c3ncc4c(N5CCCC(O)C5)nc(OC[C@@]56CCCN5C[C@H](F)C6)nc4c3F)c12]

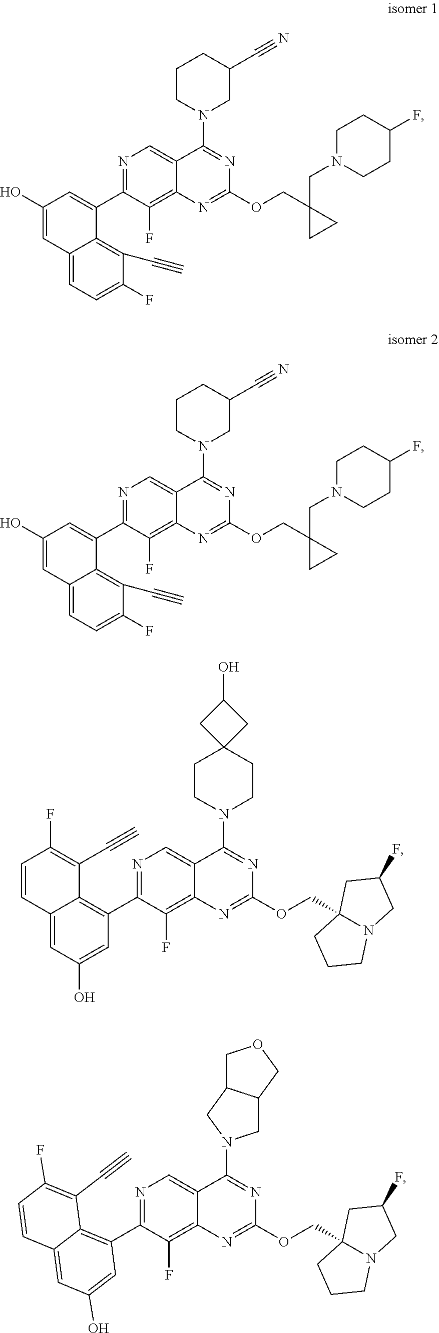 [SMILES: C#Cc1c(F)ccc2cc(O)cc(-c3ncc4c(N5CCCC(C#N)C5)nc(OCC5(CN6CCC(F)CC6)CC5)nc4c3F)c12 ; C#Cc1c(F)ccc2cc(O)cc(-c3ncc4c(N5CCCC(C#N)C5)nc(OCC5(CN6CCC(F)CC6)CC5)nc4c3F)c12 ; C#Cc1c(F)ccc2cc(O)cc(-c3ncc4c(N5CCC6(CC5)CC(O)C6)nc(OC[C@@]56CCCN5C[C@H](F)C6)nc4c3F)c12 ; C#Cc1c(F)ccc2cc(O)cc(-c3ncc4c(N5CC6COCC6C5)nc(OC[C@@]56CCCN5C[C@H](F)C6)nc4c3F)c12]

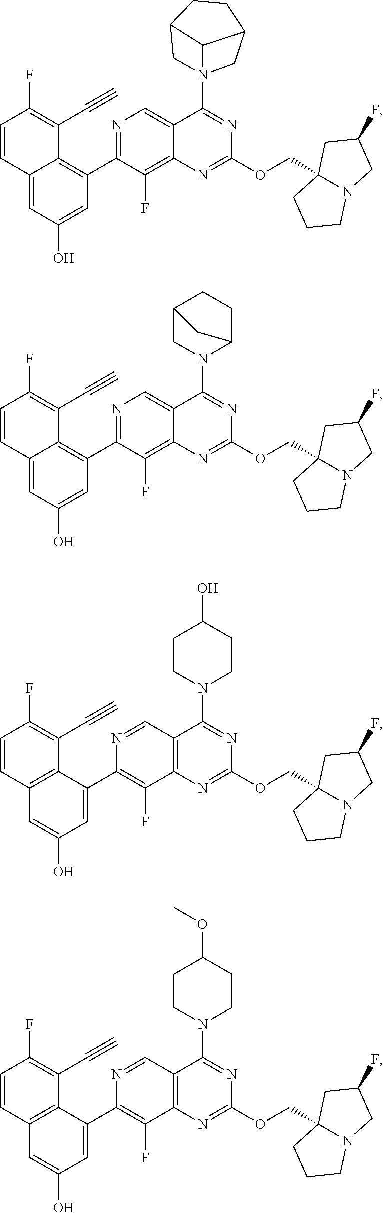

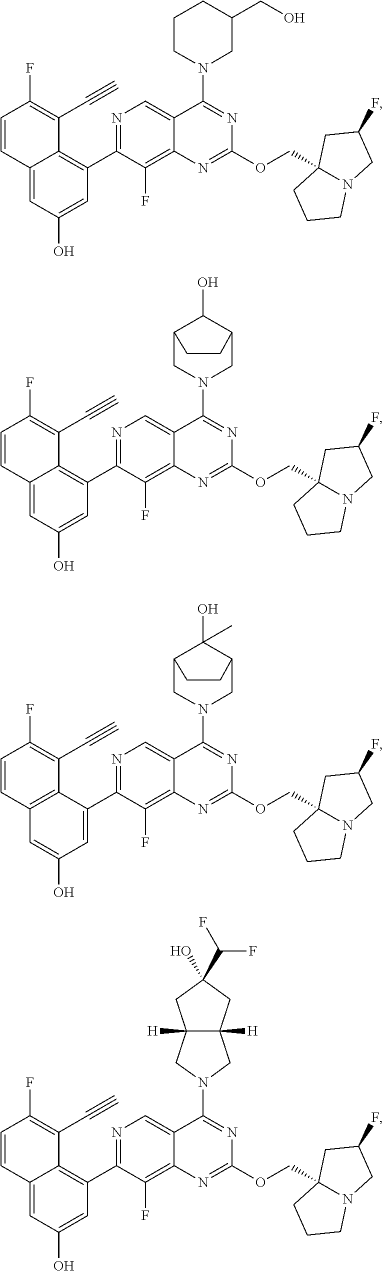 [SMILES: C#Cc1c(F)ccc2cc(O)cc(-c3ncc4c(N5CCCC(CO)C5)nc(OC[C@@]56CCCN5C[C@H](F)C6)nc4c3F)c12 ; C#Cc1c(F)ccc2cc(O)cc(-c3ncc4c(N5CC6CCC(C5)C6O)nc(OC[C@@]56CCCN5C[C@H](F)C6)nc4c3F)c12 ; C#Cc1c(F)ccc2cc(O)cc(-c3ncc4c(N5CC6CCC(C5)C6(C)O)nc(OC[C@@]56CCCN5C[C@H](F)C6)nc4c3F)c12 ; C#Cc1c(F)ccc2cc(O)cc(-c3ncc4c(N5C[C@@H]6C[C@@](O)(C(F)F)C[C@@H]6C5)nc(OC[C@@]56CCCN5C[C@H](F)C6)nc4c3F)c12]

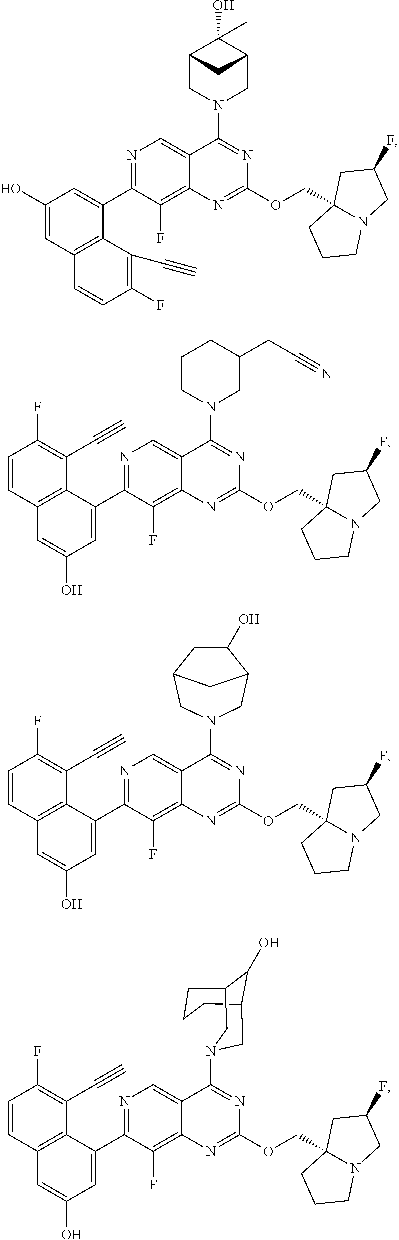 [SMILES: C#Cc1c(F)ccc2cc(O)cc(-c3ncc4c(N5C[C@H]6C[C@@H](C5)[C@@]6(C)O)nc(OC[C@@]56CCCN5C[C@H](F)C6)nc4c3F)c12 ; C#Cc1c(F)ccc2cc(O)cc(-c3ncc4c(N5CCCC(CC#N)C5)nc(OC[C@@]56CCCN5C[C@H](F)C6)nc4c3F)c12 ; C#Cc1c(F)ccc2cc(O)cc(-c3ncc4c(N5CC6CC(O)C(C6)C5)nc(OC[C@@]56CCCN5C[C@H](F)C6)nc4c3F)c12 ; C#Cc1c(F)ccc2cc(O)cc(-c3ncc4c(N5CC6CCCC(C5)C6O)nc(OC[C@@]56CCCN5C[C@H](F)C6)nc4c3F)c12]

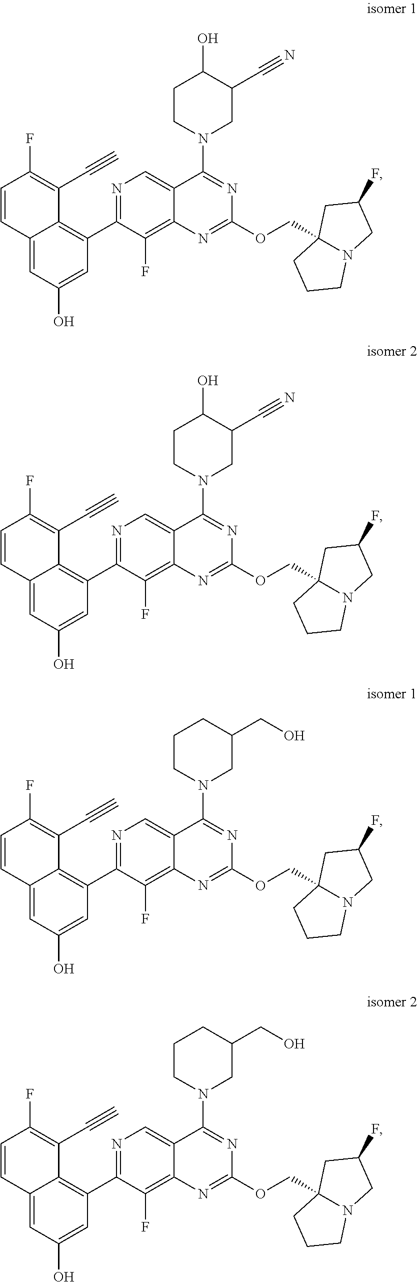 [SMILES: C#Cc1c(F)ccc2cc(O)cc(-c3ncc4c(N5CCC(O)C(C#N)C5)nc(OC[C@@]56CCCN5C[C@H](F)C6)nc4c3F)c12 ; C#Cc1c(F)ccc2cc(O)cc(-c3ncc4c(N5CCC(O)C(C#N)C5)nc(OC[C@@]56CCCN5C[C@H](F)C6)nc4c3F)c12 ; C#Cc1c(F)ccc2cc(O)cc(-c3ncc4c(N5CCCC(CO)C5)nc(OC[C@@]56CCCN5C[C@H](F)C6)nc4c3F)c12 ; C#Cc1c(F)ccc2cc(O)cc(-c3ncc4c(N5CCCC(CO)C5)nc(OC[C@@]56CCCN5C[C@H](F)C6)nc4c3F)c12]

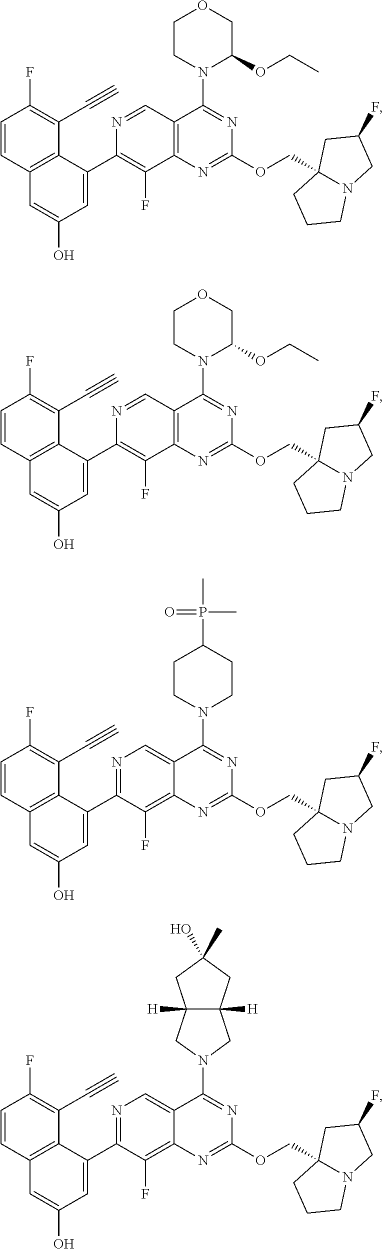 [SMILES: C#Cc1c(F)ccc2cc(O)cc(-c3ncc4c(N5CCOC[C@H]5OCC)nc(OC[C@@]56CCCN5C[C@H](F)C6)nc4c3F)c12 ; C#Cc1c(F)ccc2cc(O)cc(-c3ncc4c(N5CCOC[C@@H]5OCC)nc(OC[C@@]56CCCN5C[C@H](F)C6)nc4c3F)c12 ; C#Cc1c(F)ccc2cc(O)cc(-c3ncc4c(N5CCC(P(C)(C)=O)CC5)nc(OC[C@@]56CCCN5C[C@H](F)C6)nc4c3F)c12 ; C#Cc1c(F)ccc2cc(O)cc(-c3ncc4c(N5C[C@@H]6C[C@](C)(O)C[C@@H]6C5)nc(OC[C@@]56CCCN5C[C@H](F)C6)nc4c3F)c12]

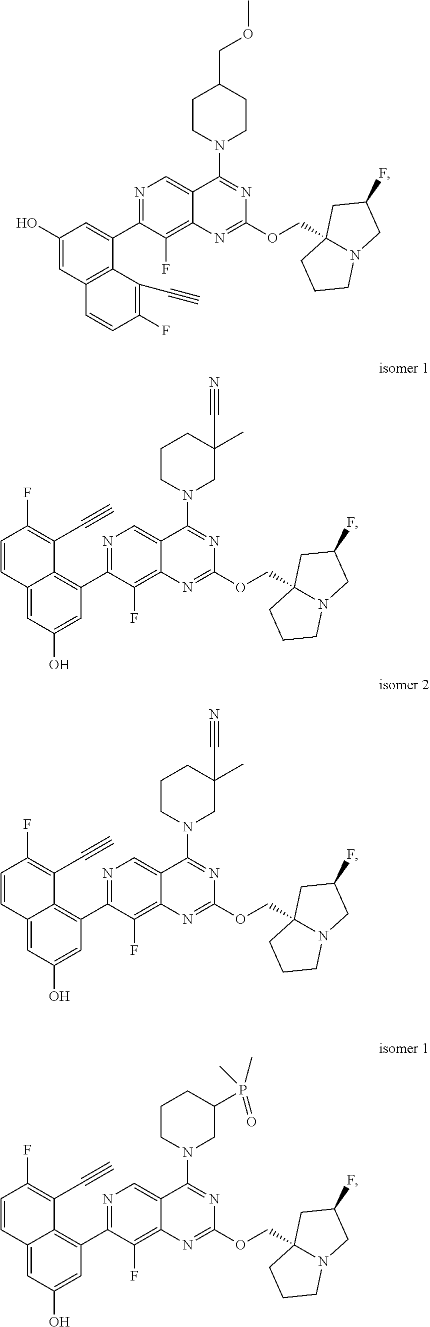 [SMILES: C#Cc1c(F)ccc2cc(O)cc(-c3ncc4c(N5CCC(COC)CC5)nc(OC[C@@]56CCCN5C[C@H](F)C6)nc4c3F)c12 ; C#Cc1c(F)ccc2cc(O)cc(-c3ncc4c(N5CCCC(C)(C#N)C5)nc(OC[C@@]56CCCN5C[C@H](F)C6)nc4c3F)c12 ; C#Cc1c(F)ccc2cc(O)cc(-c3ncc4c(N5CCCC(C)(C#N)C5)nc(OC[C@@]56CCCN5C[C@H](F)C6)nc4c3F)c12 ; C#Cc1c(F)ccc2cc(O)cc(-c3ncc4c(N5CCCC(P(C)(C)=O)C5)nc(OC[C@@]56CCCN5C[C@H](F)C6)nc4c3F)c12]

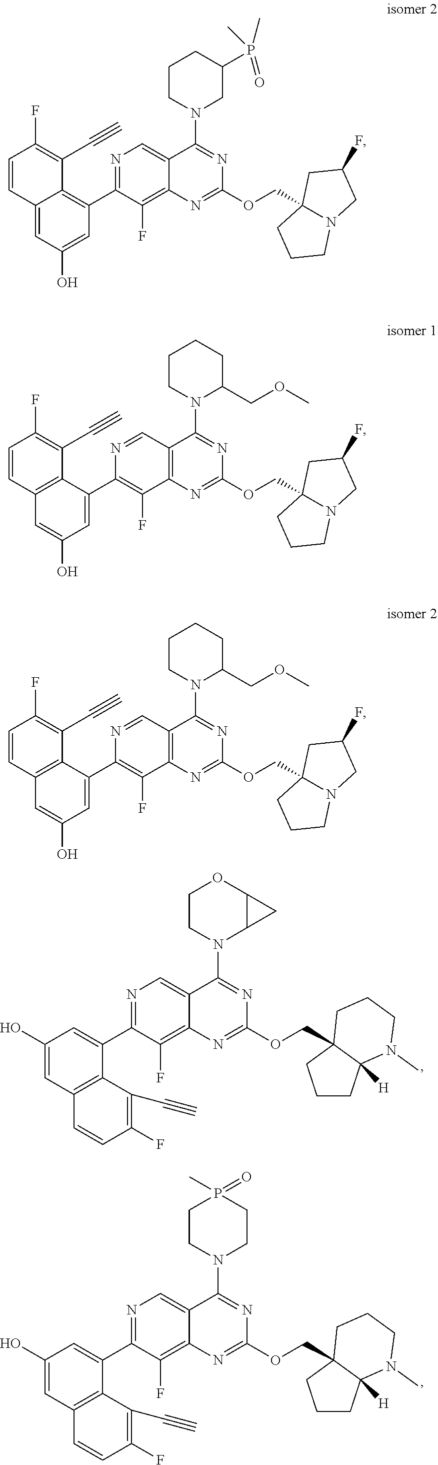

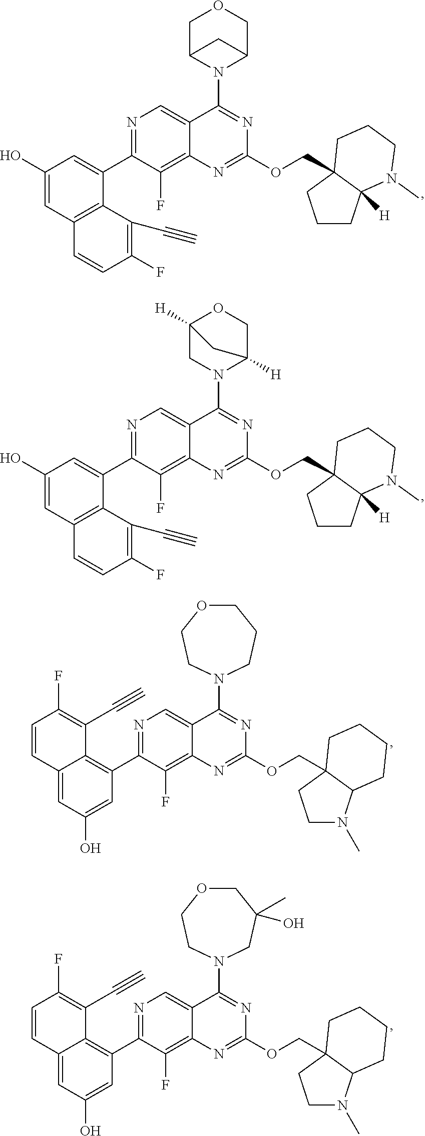

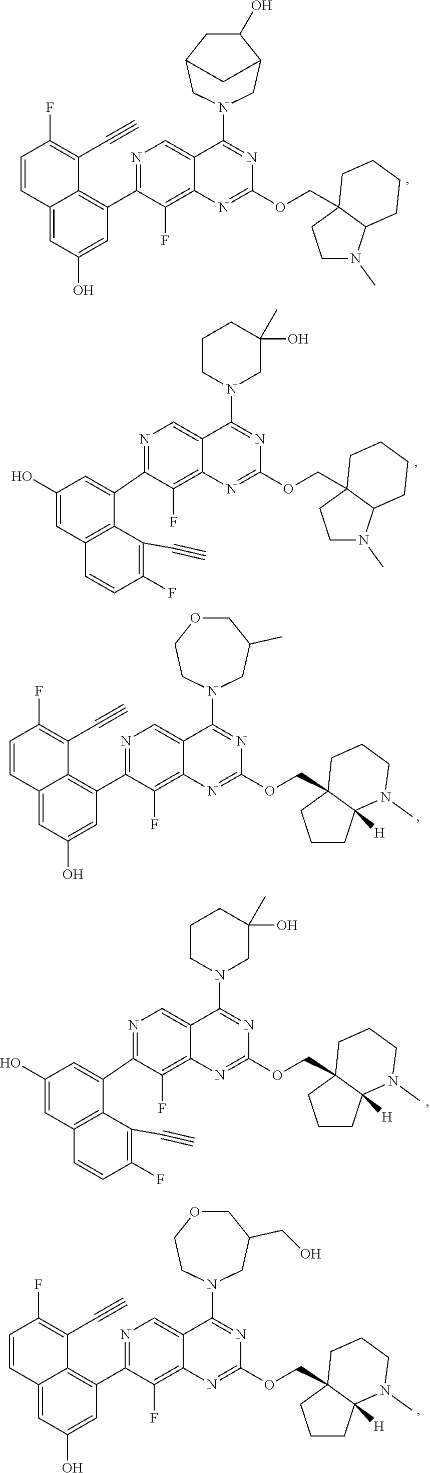

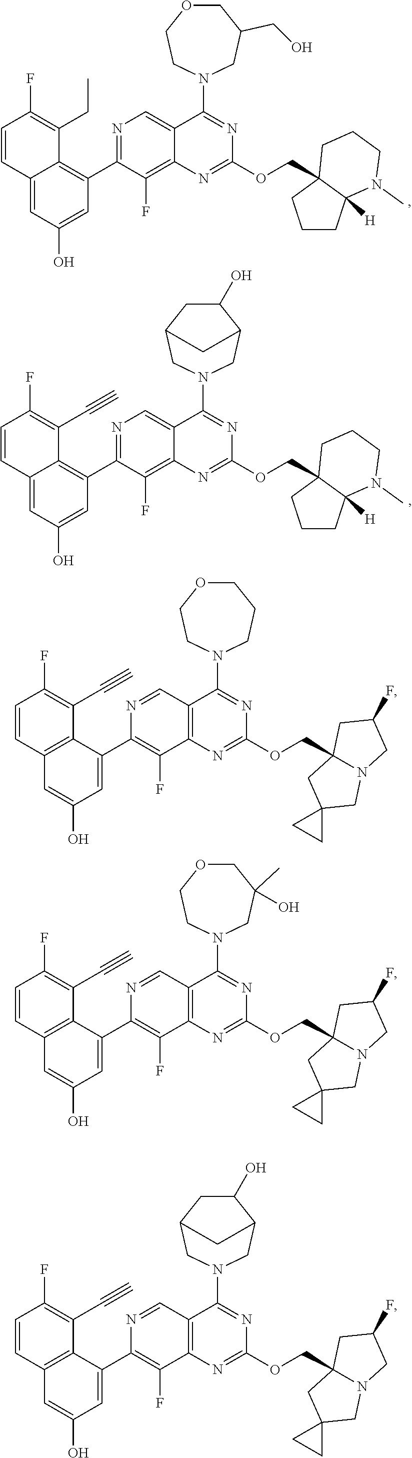

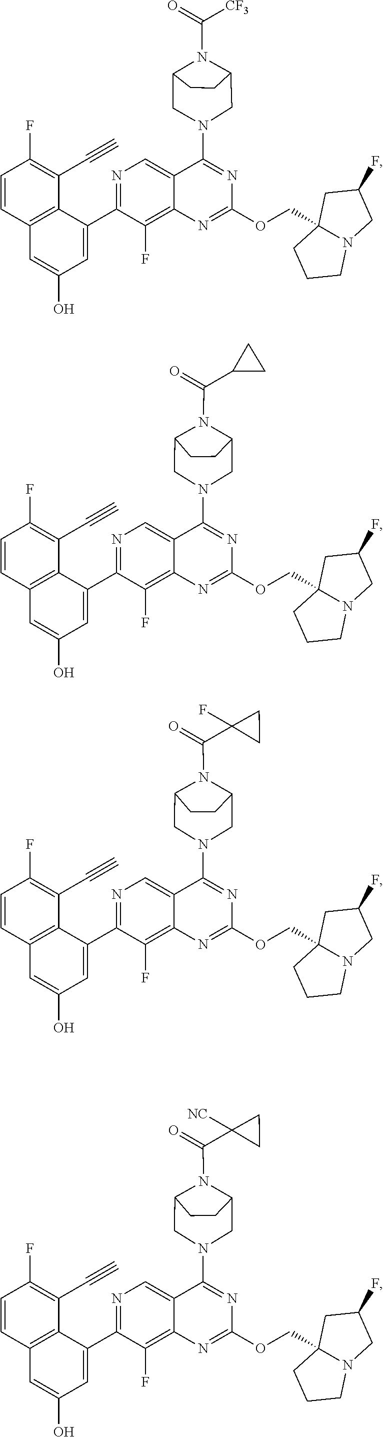 [SMILES: C#Cc1c(F)ccc2cc(O)cc(-c3ncc4c(N5CC6CCC(C5)N6C(=O)C(F)(F)F)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)C5CC5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)C5(F)CC5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)C5(C#N)CC5)nc(OC[C@@]56CCCN5C[C@H](F)C6)nc4c3F)c12]

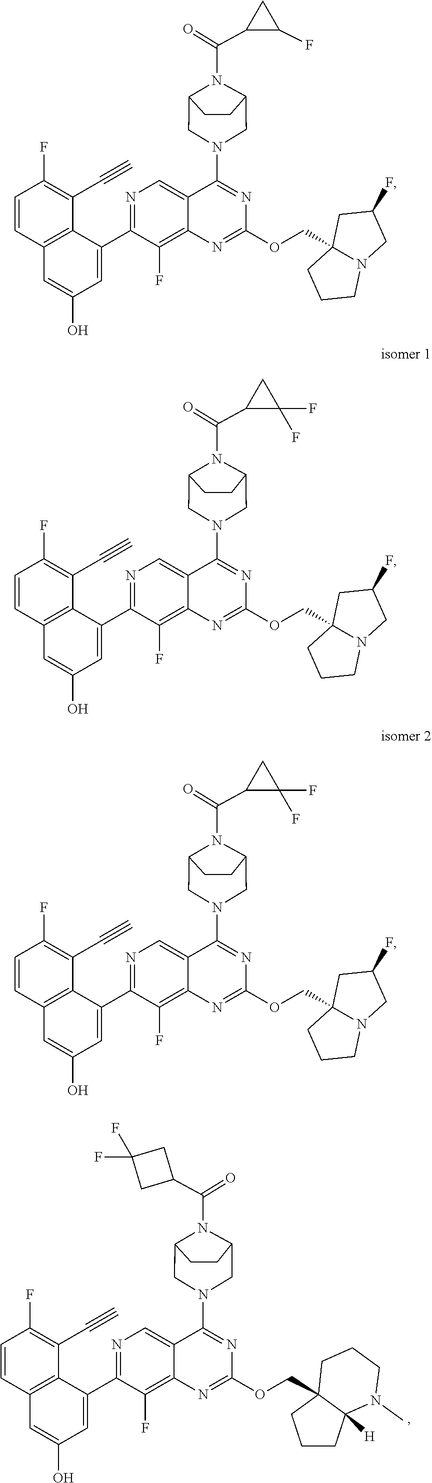 [SMILES: C#Cc1c(F)ccc2cc(O)cc(-c3ncc4c(N5CC6CCC(C5)N6C(=O)C5CC5F)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)C5CC5(F)F)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)C5CC5(F)F)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)C5CC(F)(F)C5)nc(OC[C@]56CCC[C@H]5N(C)CCC6)nc4c3F)c12]

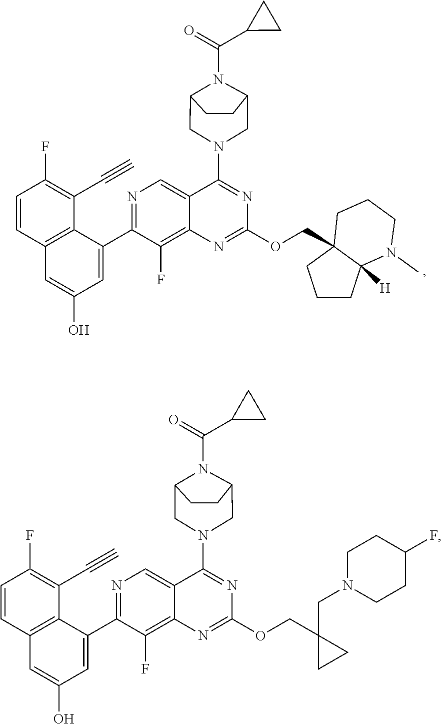 [SMILES: C#Cc1c(F)ccc2cc(O)cc(-c3ncc4c(N5CC6CCC(C5)N6C(=O)C5CC5)nc(OC[C@]56CCC[C@H]5N(C)CCC6)nc4c3F)c12 ; C#Cc1c(F)ccc2cc(O)cc(-c3ncc4c(N5CC6CCC(C5)N6C(=O)C5CC5)nc(OCC5(CN6CCC(F)CC6)CC5)nc4c3F)c12]

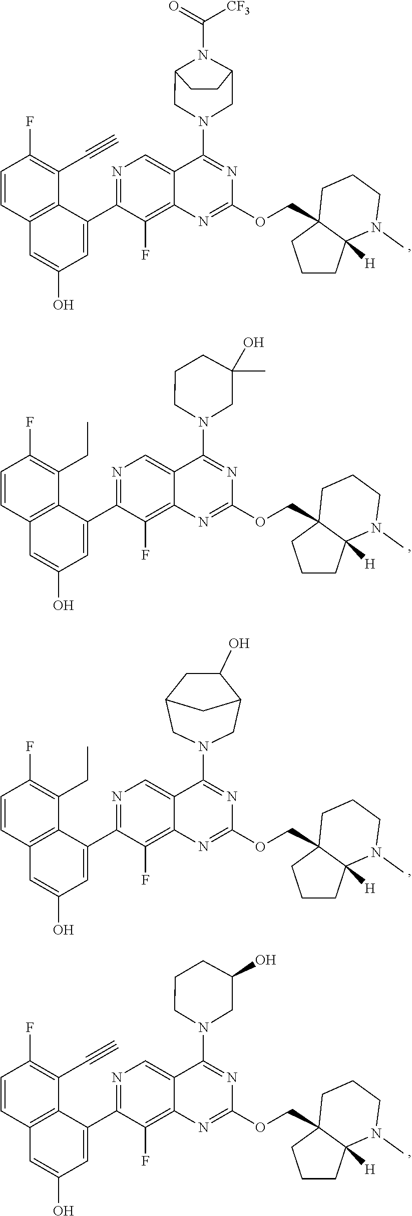 [SMILES: C#Cc1c(F)ccc2cc(O)cc(-c3ncc4c(N5CC6CCC(C5)N6C(=O)C(F)(F)F)nc(OC[C@]56CCC[C@H]5N(C)CCC6)nc4c3F)c12 ; CCc1c(F)ccc2cc(O)cc(-c3ncc4c(N5CCCC(C)(O)C5)nc(OC[C@]56CCC[C@H]5N(C)CCC6)nc4c3F)c12 ; CCc1c(F)ccc2cc(O)cc(-c3ncc4c(N5CC6CC(O)C(C6)C5)nc(OC[C@]56CCC[C@H]5N(C)CCC6)nc4c3F)c12 ; C#Cc1c(F)ccc2cc(O)cc(-c3ncc4c(N5CCC[C@@H](O)C5)nc(OC[C@]56CCC[C@H]5N(C)CCC6)nc4c3F)c12]

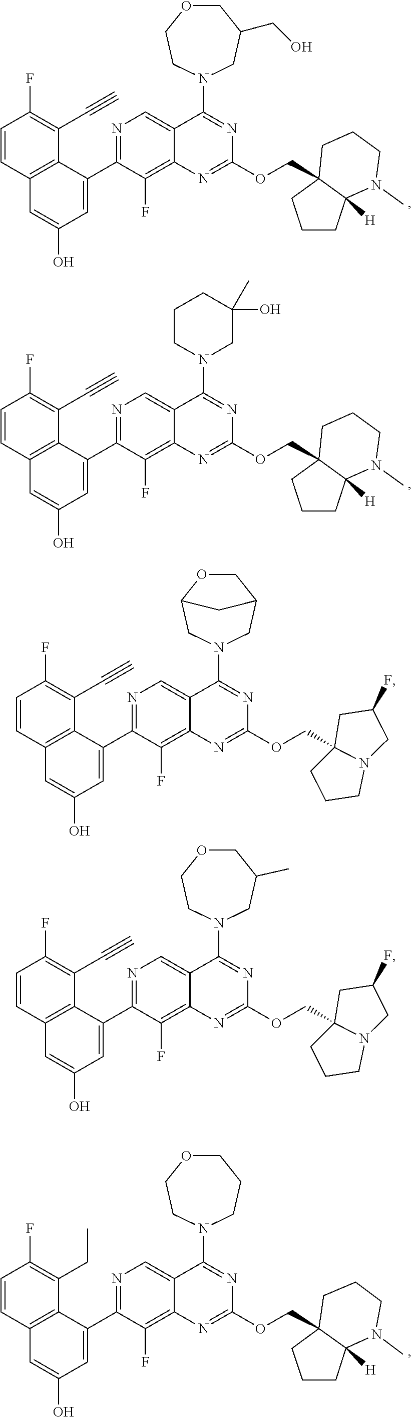 [SMILES: C#Cc1c(F)ccc2cc(O)cc(-c3ncc4c(N5CCOCC(CO)C5)nc(OC[C@]56CCC[C@H]5N(C)CCC6)nc4c3F)c12 ; C#Cc1c(F)ccc2cc(O)cc(-c3ncc4c(N5CCCC(C)(O)C5)nc(OC[C@]56CCC[C@H]5N(C)CCC6)nc4c3F)c12 ; C#Cc1c(F)ccc2cc(O)cc(-c3ncc4c(N5CC6COC(C6)C5)nc(OC[C@@]56CCCN5C[C@H](F)C6)nc4c3F)c12 ; C#Cc1c(F)ccc2cc(O)cc(-c3ncc4c(N5CCOCC(C)C5)nc(OC[C@@]56CCCN5C[C@H](F)C6)nc4c3F)c12 ; CCc1c(F)ccc2cc(O)cc(-c3ncc4c(N5CCCOCC5)nc(OC[C@]56CCC[C@H]5N(C)CCC6)nc4c3F)c12]

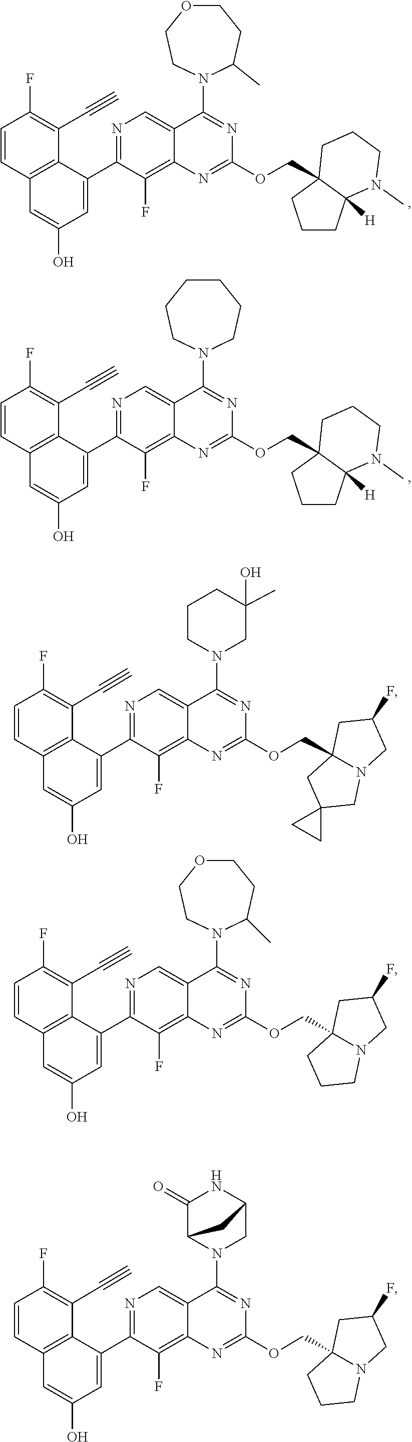 [SMILES: C#Cc1c(F)ccc2cc(O)cc(-c3ncc4c(N5CCOCCC5C)nc(OC[C@]56CCC[C@H]5N(C)CCC6)nc4c3F)c12 ; C#Cc1c(F)ccc2cc(O)cc(-c3ncc4c(N5CCCCCC5)nc(OC[C@]56CCC[C@H]5N(C)CCC6)nc4c3F)c12 ; C#Cc1c(F)ccc2cc(O)cc(-c3ncc4c(N5CCCC(C)(O)C5)nc(OC[C@@]56C[C@@H](F)CN5CC5(CC5)C6)nc4c3F)c12 ; C#Cc1c(F)ccc2cc(O)cc(-c3ncc4c(N5CCOCCC5C)nc(OC[C@@]56CCCN5C[C@H](F)C6)nc4c3F)c12 ; C#Cc1c(F)ccc2cc(O)cc(-c3ncc4c(N5C[C@@H]6C[C@H]5C(=O)N6)nc(OC[C@@]56CCCN5C[C@H](F)C6)nc4c3F)c12]

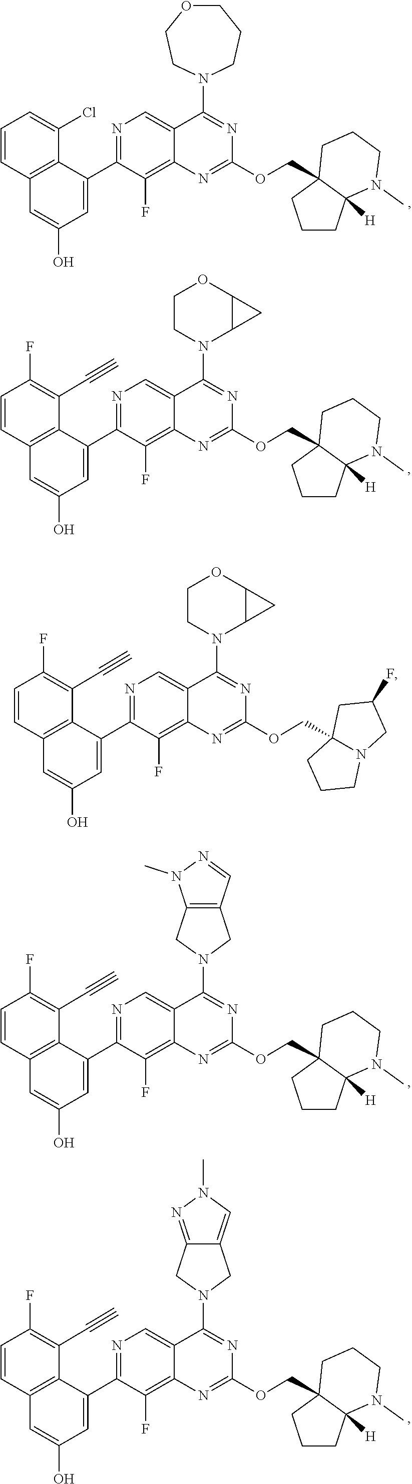 [SMILES: CN1CCC[C@@]2(COc3nc(N4CCCOCC4)c4cnc(-c5cc(O)cc6cccc(Cl)c56)c(F)c4n3)CCC[C@@H]12 ; C#Cc1c(F)ccc2cc(O)cc(-c3ncc4c(N5CCOC6CC65)nc(OC[C@]56CCC[C@H]5N(C)CCC6)nc4c3F)c12 ; C#Cc1c(F)ccc2cc(O)cc(-c3ncc4c(N5CCOC6CC65)nc(OC[C@@]56CCCN5C[C@H](F)C6)nc4c3F)c12 ; C#Cc1c(F)ccc2cc(O)cc(-c3ncc4c(N5Cc6cnn(C)c6C5)nc(OC[C@]56CCC[C@H]5N(C)CCC6)nc4c3F)c12 ; C#Cc1c(F)ccc2cc(O)cc(-c3ncc4c(N5Cc6cn(C)nc6C5)nc(OC[C@]56CCC[C@H]5N(C)CCC6)nc4c3F)c12]

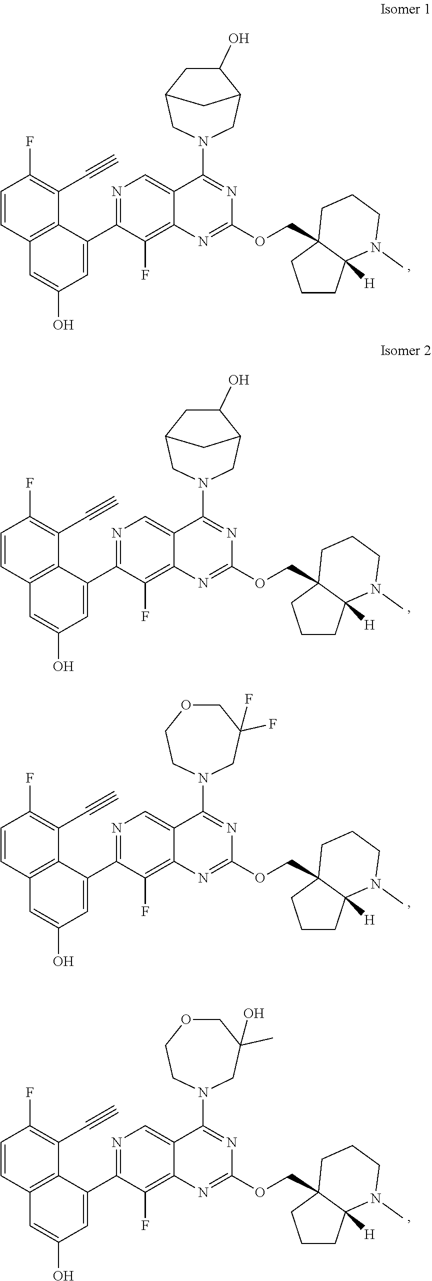 [SMILES: C#Cc1c(F)ccc2cc(O)cc(-c3ncc4c(N5CC6CC(O)C(C6)C5)nc(OC[C@]56CCC[C@H]5N(C)CCC6)nc4c3F)c12 ; C#Cc1c(F)ccc2cc(O)cc(-c3ncc4c(N5CC6CC(O)C(C6)C5)nc(OC[C@]56CCC[C@H]5N(C)CCC6)nc4c3F)c12 ; C#Cc1c(F)ccc2cc(O)cc(-c3ncc4c(N5CCOCC(F)(F)C5)nc(OC[C@]56CCC[C@H]5N(C)CCC6)nc4c3F)c12 ; C#Cc1c(F)ccc2cc(O)cc(-c3ncc4c(N5CCOCC(C)(O)C5)nc(OC[C@]56CCC[C@H]5N(C)CCC6)nc4c3F)c12]

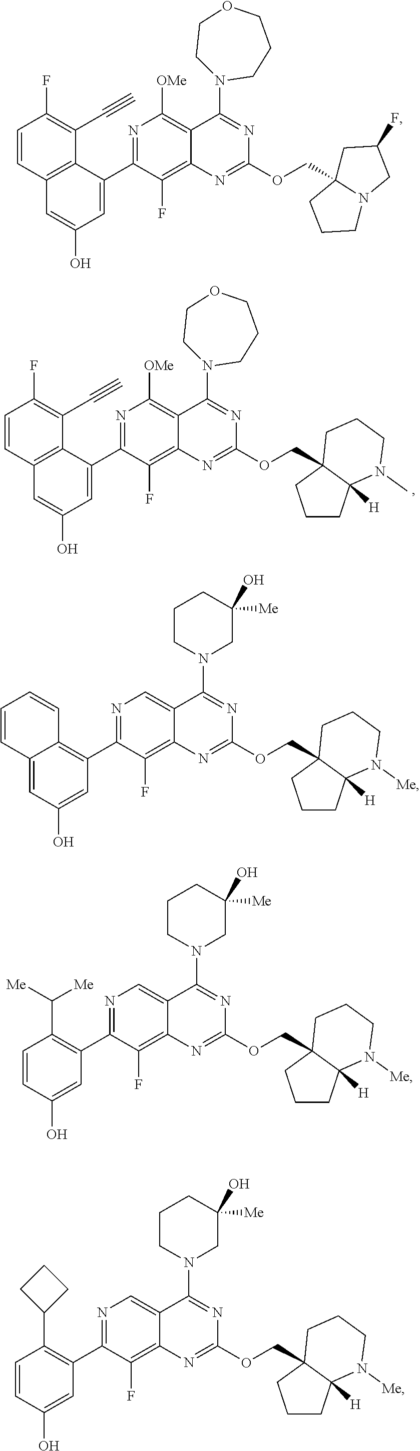 [SMILES: C#Cc1c(F)ccc2cc(O)cc(-c3nc(OC)c4c(N5CCCOCC5)nc(OC[C@@]56CCCN5C[C@H](F)C6)nc4c3F)c12 ; C#Cc1c(F)ccc2cc(O)cc(-c3nc(OC)c4c(N5CCCOCC5)nc(OC[C@]56CCC[C@H]5N(C)CCC6)nc4c3F)c12 ; CN1CCC[C@@]2(COc3nc(N4CCC[C@@](C)(O)C4)c4cnc(-c5cc(O)cc6ccccc56)c(F)c4n3)CCC[C@@H]12 ; CC(C)c1ccc(O)cc1-c1ncc2c(N3CCC[C@@](C)(O)C3)nc(OC[C@]34CCC[C@H]3N(C)CCC4)nc2c1F ; CN1CCC[C@@]2(COc3nc(N4CCC[C@@](C)(O)C4)c4cnc(-c5cc(O)ccc5C5CCC5)c(F)c4n3)CCC[C@@H]12]

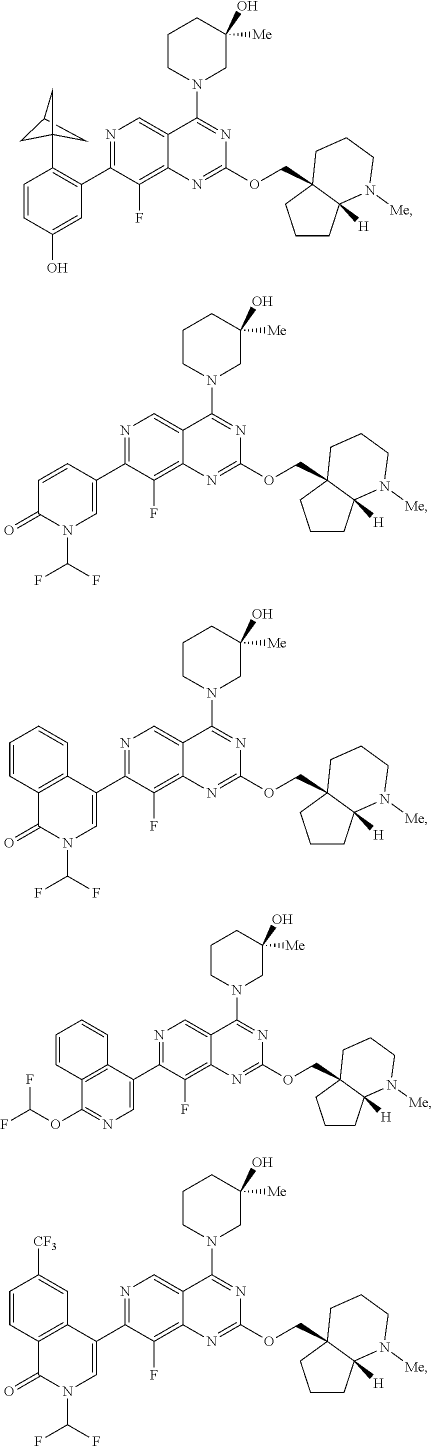 [SMILES: CN1CCC[C@@]2(COc3nc(N4CCC[C@@](C)(O)C4)c4cnc(-c5cc(O)ccc5C56CC(C5)C6)c(F)c4n3)CCC[C@@H]12 ; CN1CCC[C@@]2(COc3nc(N4CCC[C@@](C)(O)C4)c4cnc(-c5ccc(=O)n(C(F)F)c5)c(F)c4n3)CCC[C@@H]12 ; CN1CCC[C@@]2(COc3nc(N4CCC[C@@](C)(O)C4)c4cnc(-c5cn(C(F)F)c(=O)c6ccccc56)c(F)c4n3)CCC[C@@H]12 ; CN1CCC[C@@]2(COc3nc(N4CCC[C@@](C)(O)C4)c4cnc(-c5cnc(OC(F)F)c6ccccc56)c(F)c4n3)CCC[C@@H]12 ; CN1CCC[C@@]2(COc3nc(N4CCC[C@@](C)(O)C4)c4cnc(-c5cn(C(F)F)c(=O)c6ccc(C(F)(F)F)cc56)c(F)c4n3)CCC[C@@H]12]

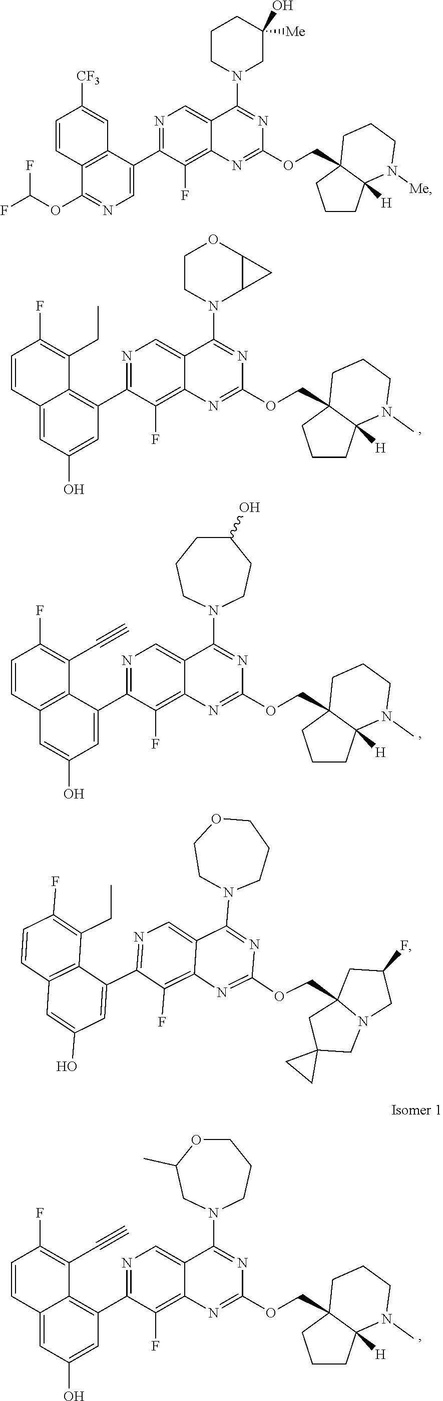 [SMILES: CN1CCC[C@@]2(COc3nc(N4CCC[C@@](C)(O)C4)c4cnc(-c5cnc(OC(F)F)c6ccc(C(F)(F)F)cc56)c(F)c4n3)CCC[C@@H]12 ; CCc1c(F)ccc2cc(O)cc(-c3ncc4c(N5CCOC6CC65)nc(OC[C@]56CCC[C@H]5N(C)CCC6)nc4c3F)c12 ; C#Cc1c(F)ccc2cc(O)cc(-c3ncc4c(N5CCCC(O)CC5)nc(OC[C@]56CCC[C@H]5N(C)CCC6)nc4c3F)c12 ; CCc1c(F)ccc2cc(O)cc(-c3ncc4c(N5CCCOCC5)nc(OC[C@@]56C[C@@H](F)CN5CC5(CC5)C6)nc4c3F)c12 ; C#Cc1c(F)ccc2cc(O)cc(-c3ncc4c(N5CCCOC(C)C5)nc(OC[C@]56CCC[C@H]5N(C)CCC6)nc4c3F)c12]

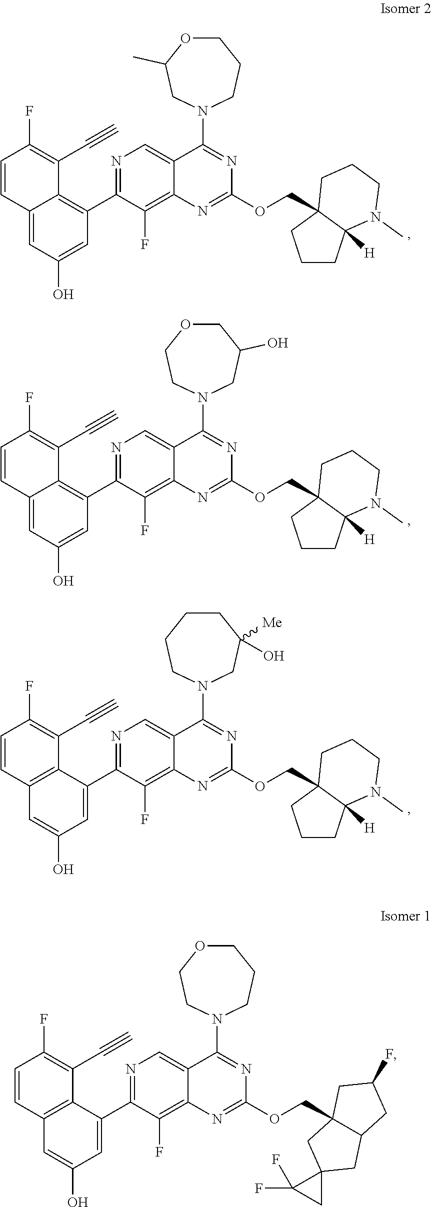 [SMILES: C#Cc1c(F)ccc2cc(O)cc(-c3ncc4c(N5CCCOC(C)C5)nc(OC[C@]56CCC[C@H]5N(C)CCC6)nc4c3F)c12 ; C#Cc1c(F)ccc2cc(O)cc(-c3ncc4c(N5CCOCC(O)C5)nc(OC[C@]56CCC[C@H]5N(C)CCC6)nc4c3F)c12 ; C#Cc1c(F)ccc2cc(O)cc(-c3ncc4c(N5CCCCC(C)(O)C5)nc(OC[C@]56CCC[C@H]5N(C)CCC6)nc4c3F)c12 ; C#Cc1c(F)ccc2cc(O)cc(-c3ncc4c(N5CCCOCC5)nc(OC[C@@]56C[C@@H](F)CC5CC5(CC5(F)F)C6)nc4c3F)c12]

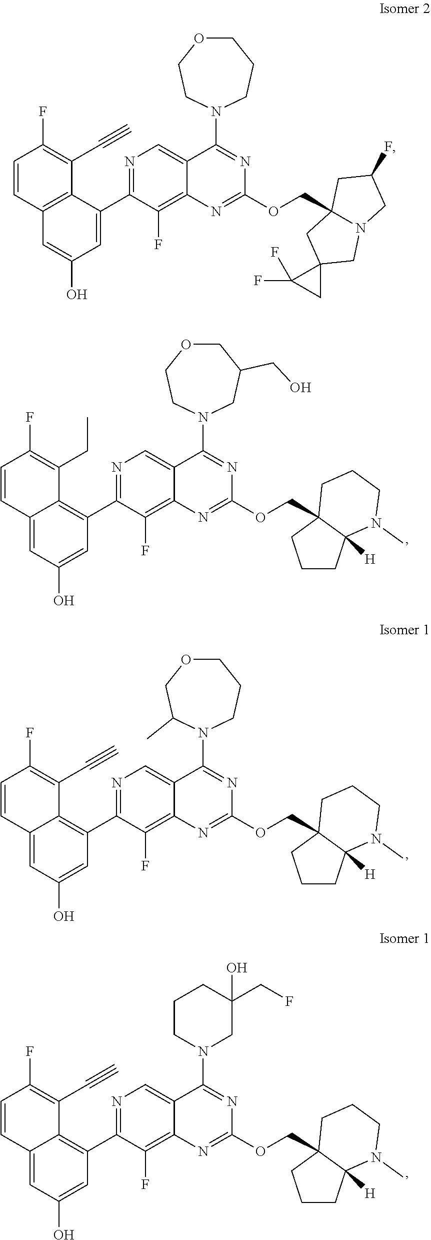 [SMILES: C#Cc1c(F)ccc2cc(O)cc(-c3ncc4c(N5CCCOCC5)nc(OC[C@@]56C[C@@H](F)CN5CC5(CC5(F)F)C6)nc4c3F)c12 ; CCc1c(F)ccc2cc(O)cc(-c3ncc4c(N5CCOCC(CO)C5)nc(OC[C@]56CCC[C@H]5N(C)CCC6)nc4c3F)c12 ; C#Cc1c(F)ccc2cc(O)cc(-c3ncc4c(N5CCCOCC5C)nc(OC[C@]56CCC[C@H]5N(C)CCC6)nc4c3F)c12 ; C#Cc1c(F)ccc2cc(O)cc(-c3ncc4c(N5CCCC(O)(CF)C5)nc(OC[C@]56CCC[C@H]5N(C)CCC6)nc4c3F)c12]

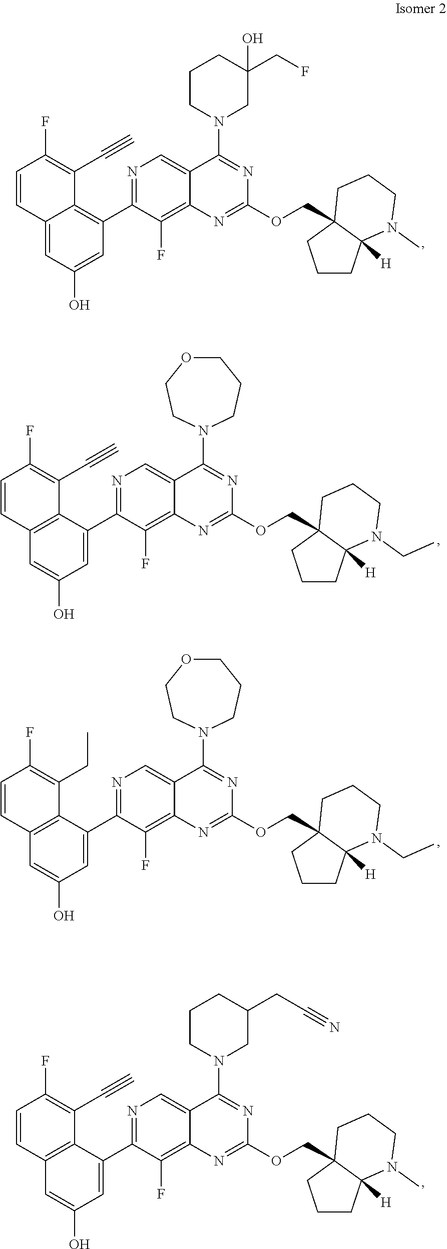 [SMILES: C#Cc1c(F)ccc2cc(O)cc(-c3ncc4c(N5CCCC(O)(CF)C5)nc(OC[C@]56CCC[C@H]5N(C)CCC6)nc4c3F)c12 ; C#Cc1c(F)ccc2cc(O)cc(-c3ncc4c(N5CCCOCC5)nc(OC[C@]56CCC[C@H]5N(CC)CCC6)nc4c3F)c12 ; CCc1c(F)ccc2cc(O)cc(-c3ncc4c(N5CCCOCC5)nc(OC[C@]56CCC[C@H]5N(CC)CCC6)nc4c3F)c12 ; C#Cc1c(F)ccc2cc(O)cc(-c3ncc4c(N5CCCC(CC#N)C5)nc(OC[C@]56CCC[C@H]5N(C)CCC6)nc4c3F)c12]

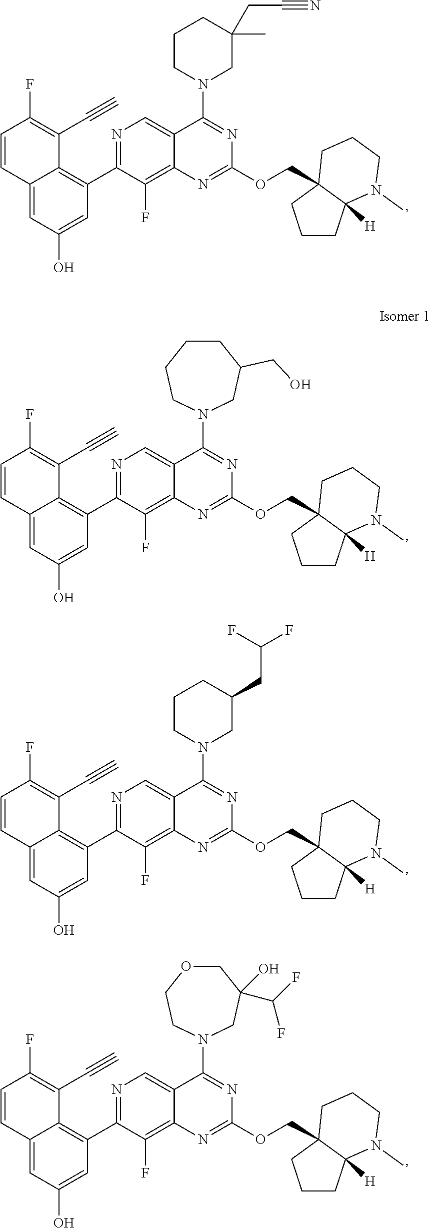 [SMILES: C#Cc1c(F)ccc2cc(O)cc(-c3ncc4c(N5CCCC(C)(CC#N)C5)nc(OC[C@]56CCC[C@H]5N(C)CCC6)nc4c3F)c12 ; C#Cc1c(F)ccc2cc(O)cc(-c3ncc4c(N5CCCCC(CO)C5)nc(OC[C@]56CCC[C@H]5N(C)CCC6)nc4c3F)c12 ; C#Cc1c(F)ccc2cc(O)cc(-c3ncc4c(N5CCC[C@@H](CC(F)F)C5)nc(OC[C@]56CCC[C@H]5N(C)CCC6)nc4c3F)c12 ; C#Cc1c(F)ccc2cc(O)cc(-c3ncc4c(N5CCOCC(O)(C(F)F)C5)nc(OC[C@]56CCC[C@H]5N(C)CCC6)nc4c3F)c12]

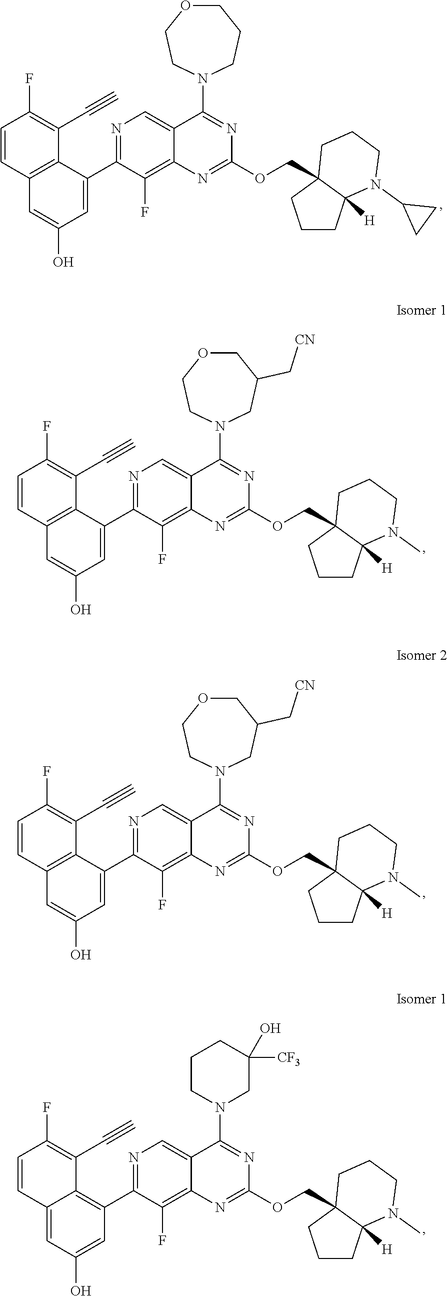 [SMILES: C#Cc1c(F)ccc2cc(O)cc(-c3ncc4c(N5CCCOCC5)nc(OC[C@]56CCC[C@H]5N(C5CC5)CCC6)nc4c3F)c12 ; C#Cc1c(F)ccc2cc(O)cc(-c3ncc4c(N5CCOCC(CC#N)C5)nc(OC[C@]56CCC[C@H]5N(C)CCC6)nc4c3F)c12 ; C#Cc1c(F)ccc2cc(O)cc(-c3ncc4c(N5CCOCC(CC#N)C5)nc(OC[C@]56CCC[C@H]5N(C)CCC6)nc4c3F)c12 ; C#Cc1c(F)ccc2cc(O)cc(-c3ncc4c(N5CCCC(O)(C(F)(F)F)C5)nc(OC[C@]56CCC[C@H]5N(C)CCC6)nc4c3F)c12]

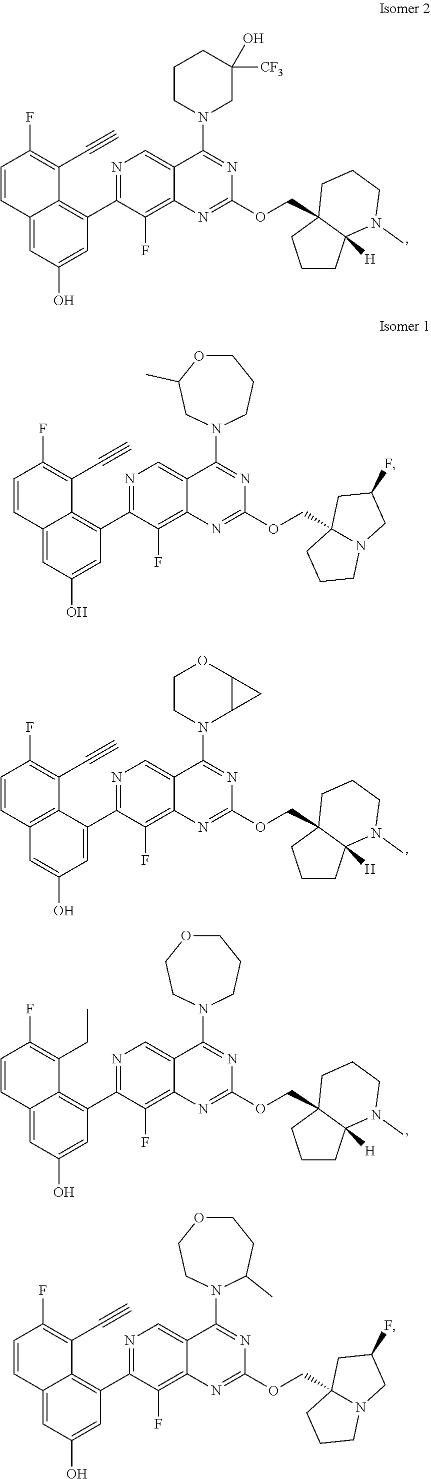 [SMILES: C#Cc1c(F)ccc2cc(O)cc(-c3ncc4c(N5CCCC(O)(C(F)(F)F)C5)nc(OC[C@]56CCC[C@H]5N(C)CCC6)nc4c3F)c12 ; C#Cc1c(F)ccc2cc(O)cc(-c3ncc4c(N5CCCOC(C)C5)nc(OC[C@@]56CCCN5C[C@H](F)C6)nc4c3F)c12 ; C#Cc1c(F)ccc2cc(O)cc(-c3ncc4c(N5CCOC6CC65)nc(OC[C@]56CCC[C@H]5N(C)CCC6)nc4c3F)c12 ; CCc1c(F)ccc2cc(O)cc(-c3ncc4c(N5CCCOCC5)nc(OC[C@]56CCC[C@H]5N(C)CCC6)nc4c3F)c12 ; C#Cc1c(F)ccc2cc(O)cc(-c3ncc4c(N5CCOCCC5C)nc(OC[C@@]56CCCN5C[C@H](F)C6)nc4c3F)c12]

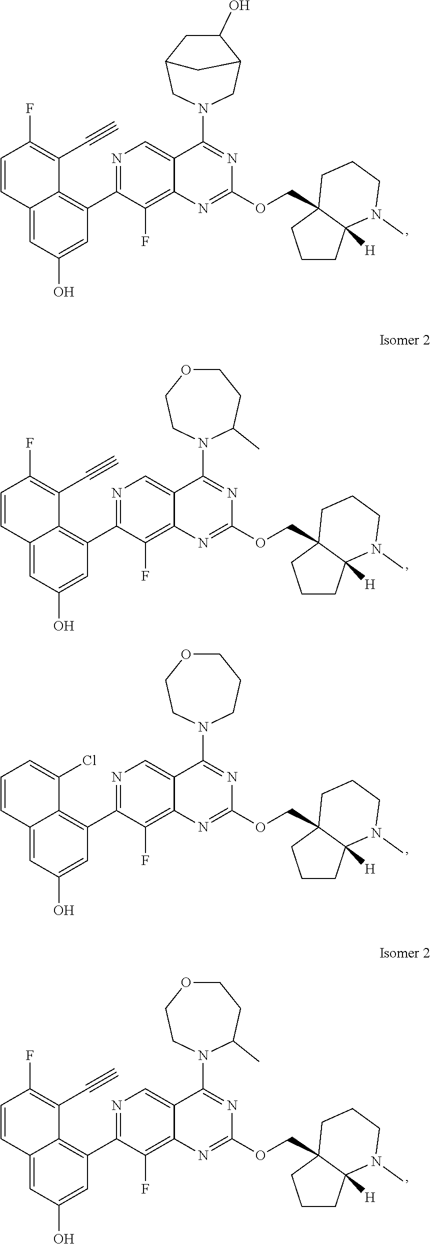 [SMILES: C#Cc1c(F)ccc2cc(O)cc(-c3ncc4c(N5CC6CC(O)C(C6)C5)nc(OC[C@]56CCC[C@H]5N(C)CCC6)nc4c3F)c12 ; C#Cc1c(F)ccc2cc(O)cc(-c3ncc4c(N5CCOCCC5C)nc(OC[C@]56CCC[C@H]5N(C)CCC6)nc4c3F)c12 ; CN1CCC[C@@]2(COc3nc(N4CCCOCC4)c4cnc(-c5cc(O)cc6cccc(Cl)c56)c(F)c4n3)CCC[C@@H]12 ; C#Cc1c(F)ccc2cc(O)cc(-c3ncc4c(N5CCOCCC5C)nc(OC[C@]56CCC[C@H]5N(C)CCC6)nc4c3F)c12]

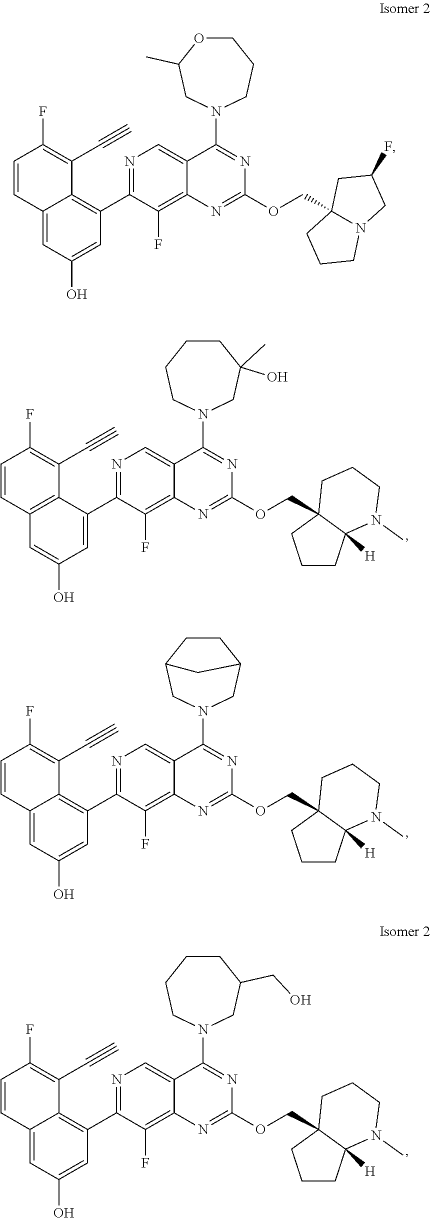 [SMILES: C#Cc1c(F)ccc2cc(O)cc(-c3ncc4c(N5CCCOC(C)C5)nc(OC[C@@]56CCCN5C[C@H](F)C6)nc4c3F)c12 ; C#Cc1c(F)ccc2cc(O)cc(-c3ncc4c(N5CCCCC(C)(O)C5)nc(OC[C@]56CCC[C@H]5N(C)CCC6)nc4c3F)c12 ; C#Cc1c(F)ccc2cc(O)cc(-c3ncc4c(N5CC6CCC(C6)C5)nc(OC[C@]56CCC[C@H]5N(C)CCC6)nc4c3F)c12 ; C#Cc1c(F)ccc2cc(O)cc(-c3ncc4c(N5CCCCC(CO)C5)nc(OC[C@]56CCC[C@H]5N(C)CCC6)nc4c3F)c12]

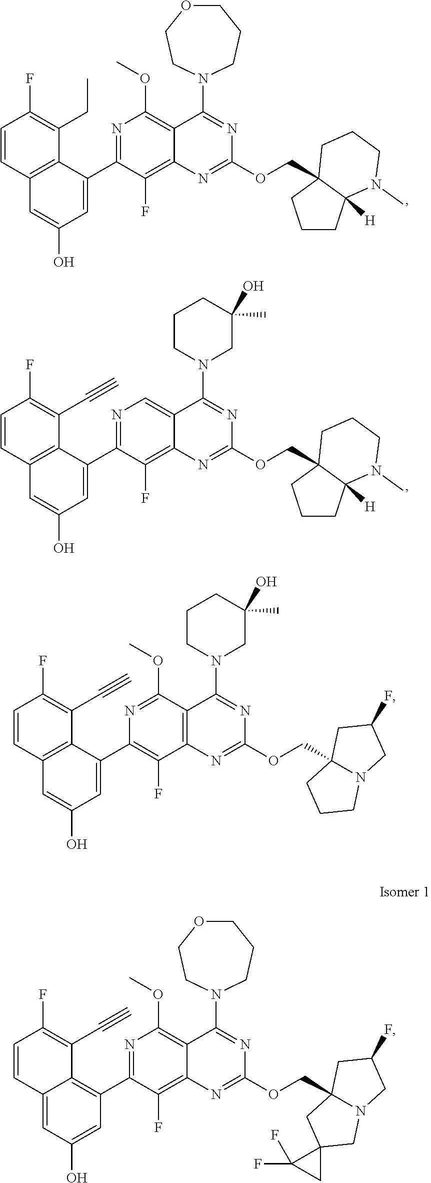 [SMILES: CCc1c(F)ccc2cc(O)cc(-c3nc(OC)c4c(N5CCCOCC5)nc(OC[C@]56CCC[C@H]5N(C)CCC6)nc4c3F)c12 ; C#Cc1c(F)ccc2cc(O)cc(-c3ncc4c(N5CCC[C@@](C)(O)C5)nc(OC[C@]56CCC[C@H]5N(C)CCC6)nc4c3F)c12 ; C#Cc1c(F)ccc2cc(O)cc(-c3nc(OC)c4c(N5CCC[C@@](C)(O)C5)nc(OC[C@@]56CCCN5C[C@H](F)C6)nc4c3F)c12 ; C#Cc1c(F)ccc2cc(O)cc(-c3nc(OC)c4c(N5CCCOCC5)nc(OC[C@@]56C[C@@H](F)CN5CC5(CC5(F)F)C6)nc4c3F)c12]

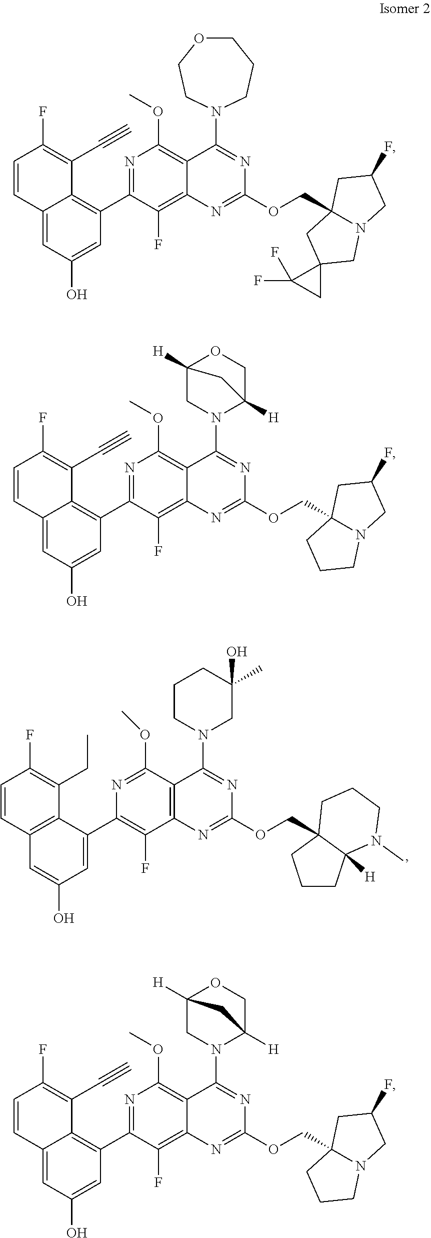 [SMILES: C#Cc1c(F)ccc2cc(O)cc(-c3nc(OC)c4c(N5CCCOCC5)nc(OC[C@@]56C[C@@H](F)CN5CC5(CC5(F)F)C6)nc4c3F)c12 ; C#Cc1c(F)ccc2cc(O)cc(-c3nc(OC)c4c(N5C[C@H]6C[C@@H]5CO6)nc(OC[C@@]56CCCN5C[C@H](F)C6)nc4c3F)c12 ; CCc1c(F)ccc2cc(O)cc(-c3nc(OC)c4c(N5CCC[C@@](C)(O)C5)nc(OC[C@]56CCC[C@H]5N(C)CCC6)nc4c3F)c12 ; C#Cc1c(F)ccc2cc(O)cc(-c3nc(OC)c4c(N5C[C@H]6C[C@@H]5CO6)nc(OC[C@@]56CCCN5C[C@H](F)C6)nc4c3F)c12]

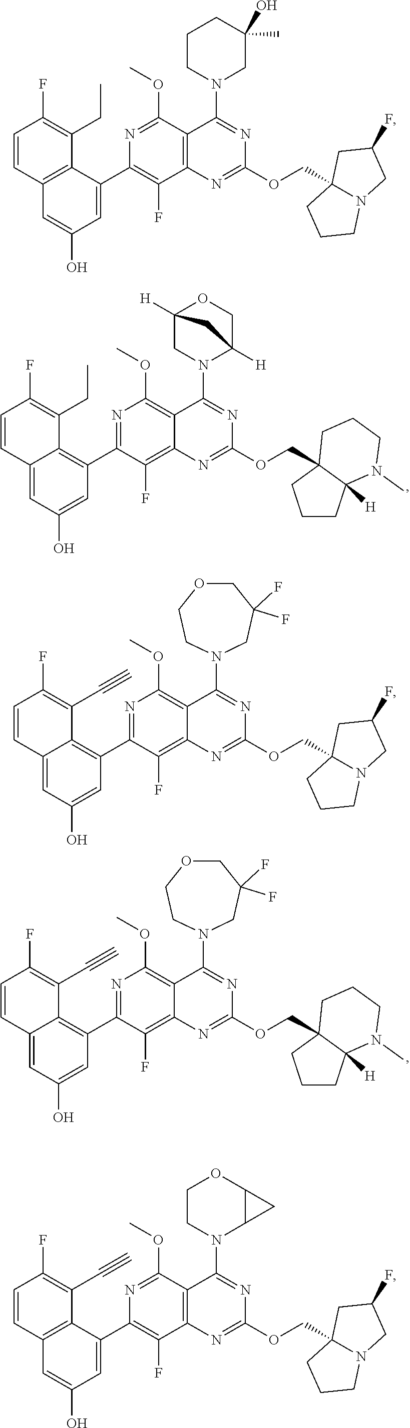 [SMILES: CCc1c(F)ccc2cc(O)cc(-c3nc(OC)c4c(N5CCC[C@@](C)(O)C5)nc(OC[C@@]56CCCN5C[C@H](F)C6)nc4c3F)c12 ; CCc1c(F)ccc2cc(O)cc(-c3nc(OC)c4c(N5C[C@H]6C[C@@H]5CO6)nc(OC[C@]56CCC[C@H]5N(C)CCC6)nc4c3F)c12 ; C#Cc1c(F)ccc2cc(O)cc(-c3nc(OC)c4c(N5CCOCC(F)(F)C5)nc(OC[C@@]56CCCN5C[C@H](F)C6)nc4c3F)c12 ; C#Cc1c(F)ccc2cc(O)cc(-c3nc(OC)c4c(N5CCOCC(F)(F)C5)nc(OC[C@]56CCC[C@H]5N(C)CCC6)nc4c3F)c12 ; C#Cc1c(F)ccc2cc(O)cc(-c3nc(OC)c4c(N5CCOC6CC65)nc(OC[C@@]56CCCN5C[C@H](F)C6)nc4c3F)c12]

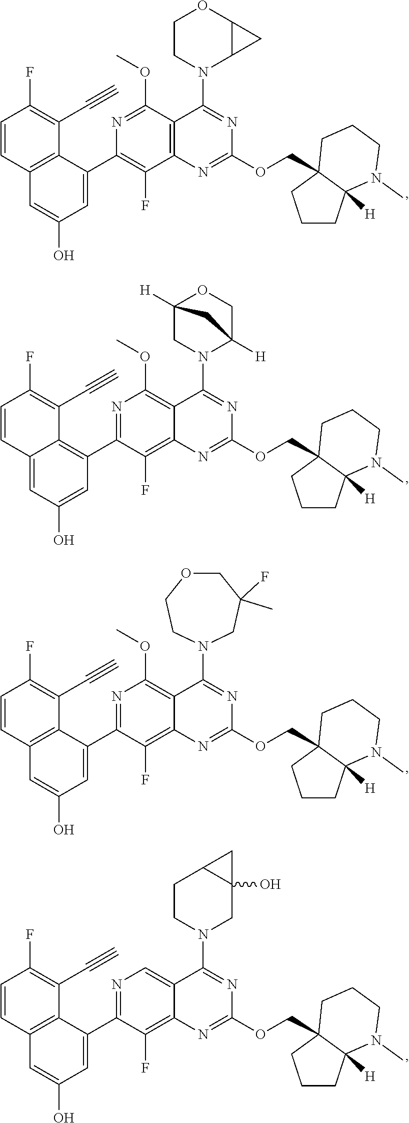 [SMILES: C#Cc1c(F)ccc2cc(O)cc(-c3nc(OC)c4c(N5CCOC6CC65)nc(OC[C@]56CCC[C@H]5N(C)CCC6)nc4c3F)c12 ; C#Cc1c(F)ccc2cc(O)cc(-c3nc(OC)c4c(N5C[C@H]6C[C@@H]5CO6)nc(OC[C@]56CCC[C@H]5N(C)CCC6)nc4c3F)c12 ; C#Cc1c(F)ccc2cc(O)cc(-c3nc(OC)c4c(N5CCOCC(C)(F)C5)nc(OC[C@]56CCC[C@H]5N(C)CCC6)nc4c3F)c12 ; C#Cc1c(F)ccc2cc(O)cc(-c3ncc4c(N5CCC6CC6(O)C5)nc(OC[C@]56CCC[C@H]5N(C)CCC6)nc4c3F)c12]

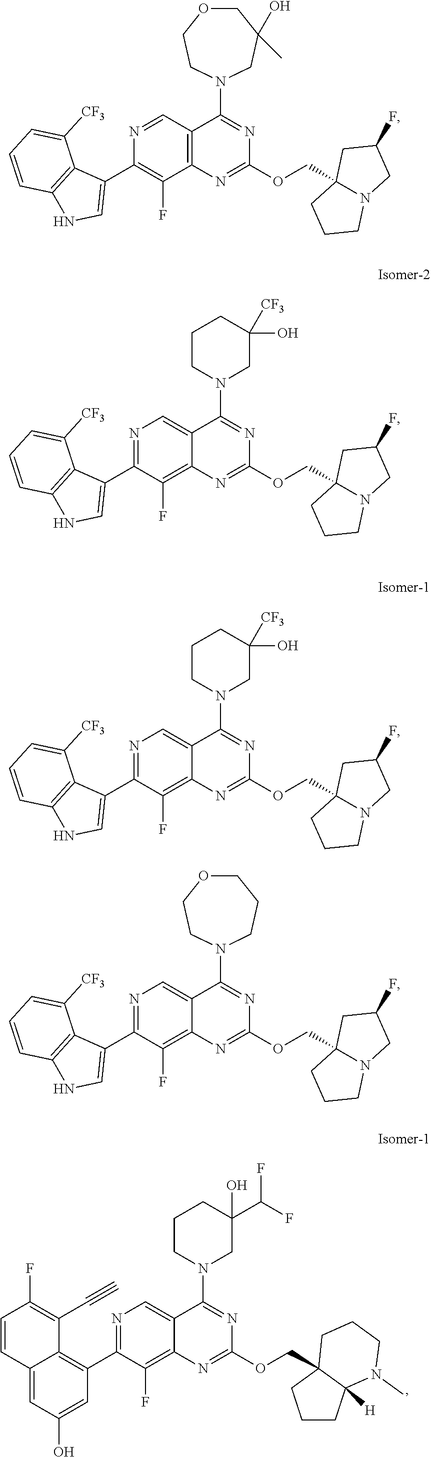 [SMILES: CC1(O)COCCN(c2nc(OC[C@@]34CCCN3C[C@H](F)C4)nc3c(F)c(-c4c[nH]c5cccc(C(F)(F)F)c45)ncc23)C1 ; OC1(C(F)(F)F)CCCN(c2nc(OC[C@@]34CCCN3C[C@H](F)C4)nc3c(F)c(-c4c[nH]c5cccc(C(F)(F)F)c45)ncc23)C1 ; OC1(C(F)(F)F)CCCN(c2nc(OC[C@@]34CCCN3C[C@H](F)C4)nc3c(F)c(-c4c[nH]c5cccc(C(F)(F)F)c45)ncc23)C1 ; Fc1c(-c2c[nH]c3cccc(C(F)(F)F)c23)ncc2c(N3CCCOCC3)nc(OC[C@@]34CCCN3C[C@H](F)C4)nc12 ; C#Cc1c(F)ccc2cc(O)cc(-c3ncc4c(N5CCCC(O)(C(F)F)C5)nc(OC[C@]56CCC[C@H]5N(C)CCC6)nc4c3F)c12]

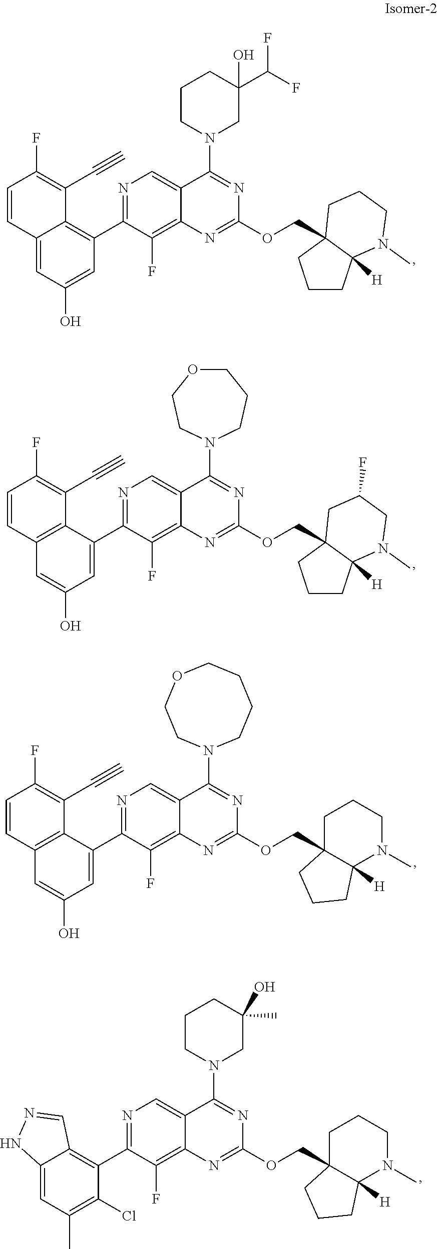 [SMILES: C#Cc1c(F)ccc2cc(O)cc(-c3ncc4c(N5CCCC(O)(C(F)F)C5)nc(OC[C@]56CCC[C@H]5N(C)CCC6)nc4c3F)c12 ; C#Cc1c(F)ccc2cc(O)cc(-c3ncc4c(N5CCCOCC5)nc(OC[C@]56CCC[C@H]5N(C)C[C@@H](F)C6)nc4c3F)c12 ; C#Cc1c(F)ccc2cc(O)cc(-c3ncc4c(N5CCCCOCC5)nc(OC[C@]56CCC[C@H]5N(C)CCC6)nc4c3F)c12 ; Cc1cc2[nH]ncc2c(-c2ncc3c(N4CCC[C@@](C)(O)C4)nc(OC[C@]45CCC[C@H]4N(C)CCC5)nc3c2F)c1Cl]

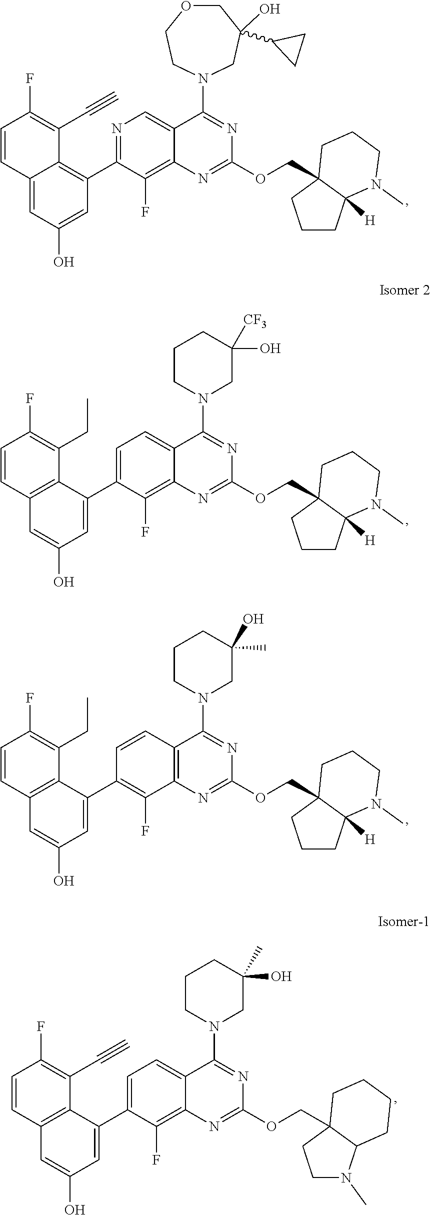 [SMILES: C#Cc1c(F)ccc2cc(O)cc(-c3ncc4c(N5CCOCC(O)(C6CC6)C5)nc(OC[C@]56CCC[C@H]5N(C)CCC6)nc4c3F)c12 ; CCc1c(F)ccc2cc(O)cc(-c3ccc4c(N5CCCC(O)(C(F)(F)F)C5)nc(OC[C@]56CCC[C@H]5N(C)CCC6)nc4c3F)c12 ; CCc1c(F)ccc2cc(O)cc(-c3ccc4c(N5CCC[C@@](C)(O)C5)nc(OC[C@]56CCC[C@H]5N(C)CCC6)nc4c3F)c12 ; C#Cc1c(F)ccc2cc(O)cc(-c3ccc4c(N5CCC[C@@](C)(O)C5)nc(OCC56CCCCC5N(C)CC6)nc4c3F)c12]

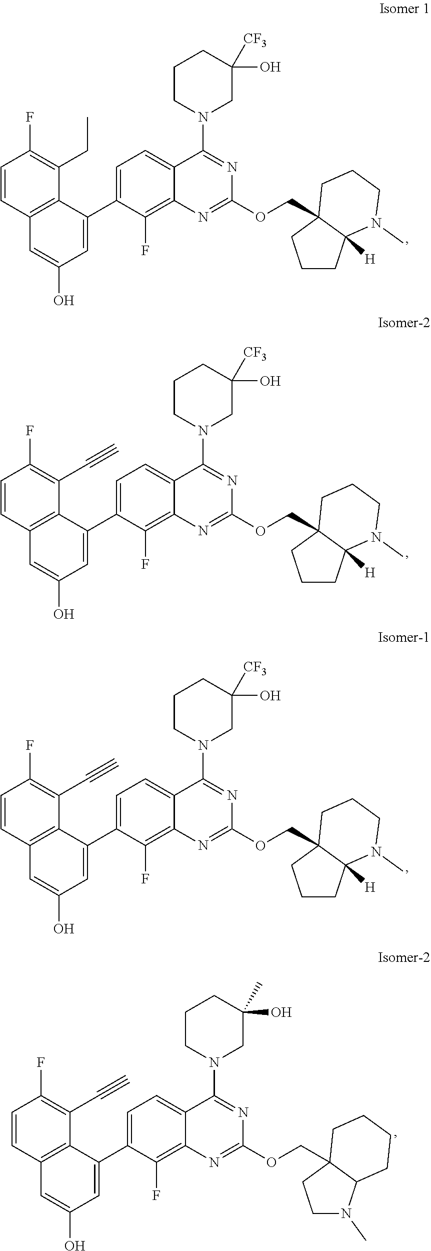 [SMILES: CCc1c(F)ccc2cc(O)cc(-c3ccc4c(N5CCCC(O)(C(F)(F)F)C5)nc(OC[C@]56CCC[C@H]5N(C)CCC6)nc4c3F)c12 ; C#Cc1c(F)ccc2cc(O)cc(-c3ccc4c(N5CCCC(O)(C(F)(F)F)C5)nc(OC[C@]56CCC[C@H]5N(C)CCC6)nc4c3F)c12 ; C#Cc1c(F)ccc2cc(O)cc(-c3ccc4c(N5CCCC(O)(C(F)(F)F)C5)nc(OC[C@]56CCC[C@H]5N(C)CCC6)nc4c3F)c12 ; C#Cc1c(F)ccc2cc(O)cc(-c3ccc4c(N5CCC[C@@](C)(O)C5)nc(OCC56CCCCC5N(C)CC6)nc4c3F)c12]

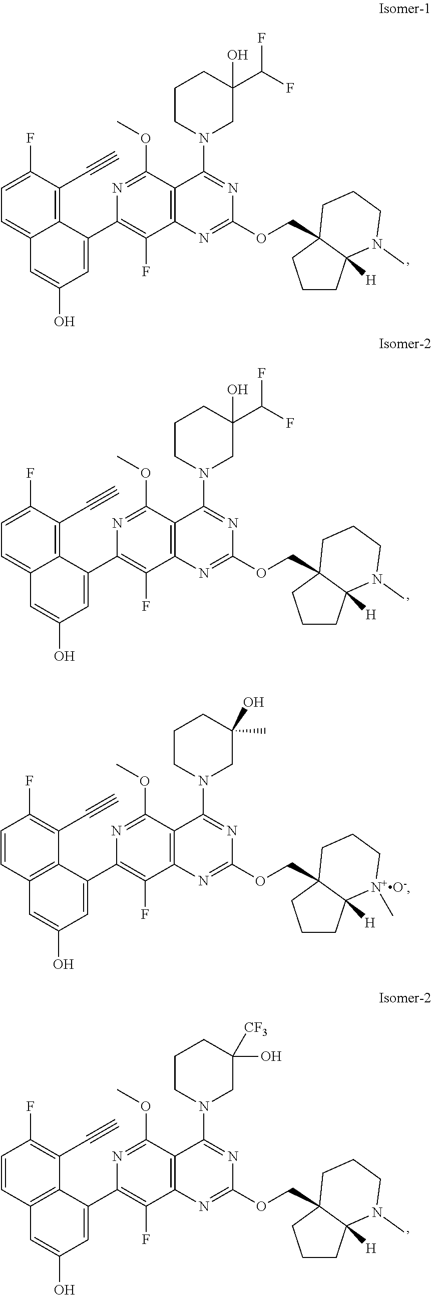

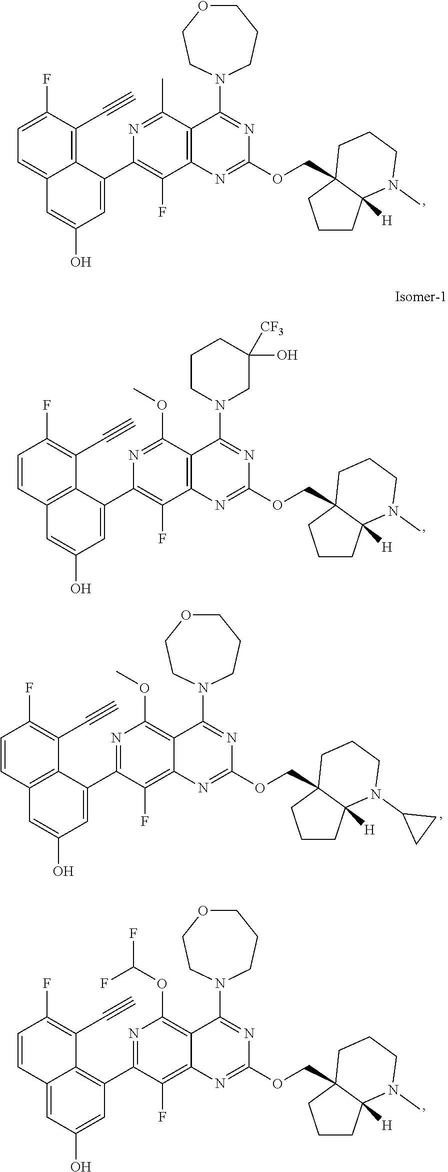 [SMILES: C#Cc1c(F)ccc2cc(O)cc(-c3nc(C)c4c(N5CCCOCC5)nc(OC[C@]56CCC[C@H]5N(C)CCC6)nc4c3F)c12 ; C#Cc1c(F)ccc2cc(O)cc(-c3nc(OC)c4c(N5CCCC(O)(C(F)(F)F)C5)nc(OC[C@]56CCC[C@H]5N(C)CCC6)nc4c3F)c12 ; C#Cc1c(F)ccc2cc(O)cc(-c3nc(OC)c4c(N5CCCOCC5)nc(OC[C@]56CCC[C@H]5N(C5CC5)CCC6)nc4c3F)c12 ; C#Cc1c(F)ccc2cc(O)cc(-c3nc(OC(F)F)c4c(N5CCCOCC5)nc(OC[C@]56CCC[C@H]5N(C)CCC6)nc4c3F)c12]

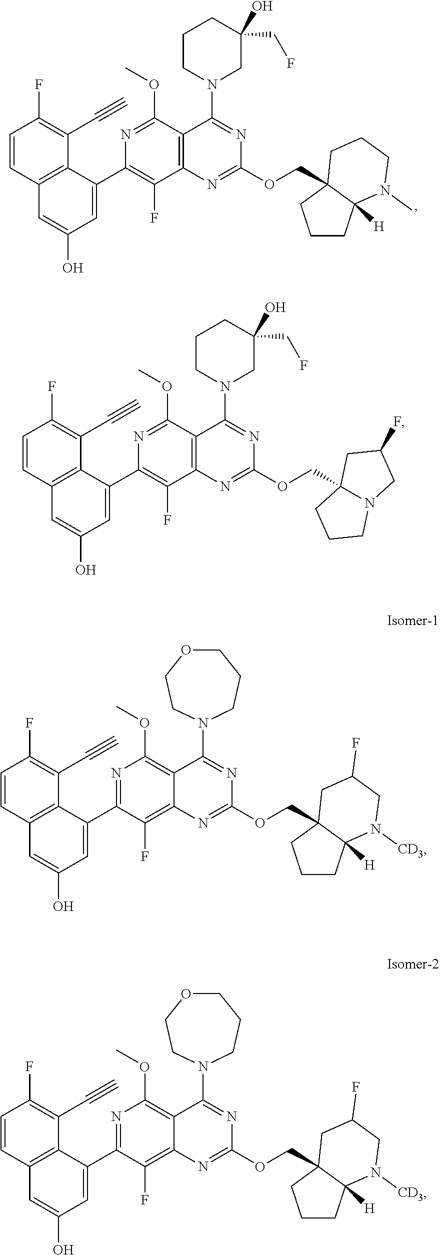 [SMILES: C#Cc1c(F)ccc2cc(O)cc(-c3nc(OC)c4c(N5CCC[C@](O)(CF)C5)nc(OC[C@]56CCC[C@H]5N(C)CCC6)nc4c3F)c12 ; C#Cc1c(F)ccc2cc(O)cc(-c3nc(OC)c4c(N5CCC[C@](O)(CF)C5)nc(OC[C@@]56CCCN5C[C@H](F)C6)nc4c3F)c12 ; [2H]C([2H])([2H])N1CC(F)C[C@@]2(COc3nc(N4CCCOCC4)c4c(OC)nc(-c5cc(O)cc6ccc(F)c(C#C)c56)c(F)c4n3)CCC[C@@H]12 ; [2H]C([2H])([2H])N1CC(F)C[C@@]2(COc3nc(N4CCCOCC4)c4c(OC)nc(-c5cc(O)cc6ccc(F)c(C#C)c56)c(F)c4n3)CCC[C@@H]12]

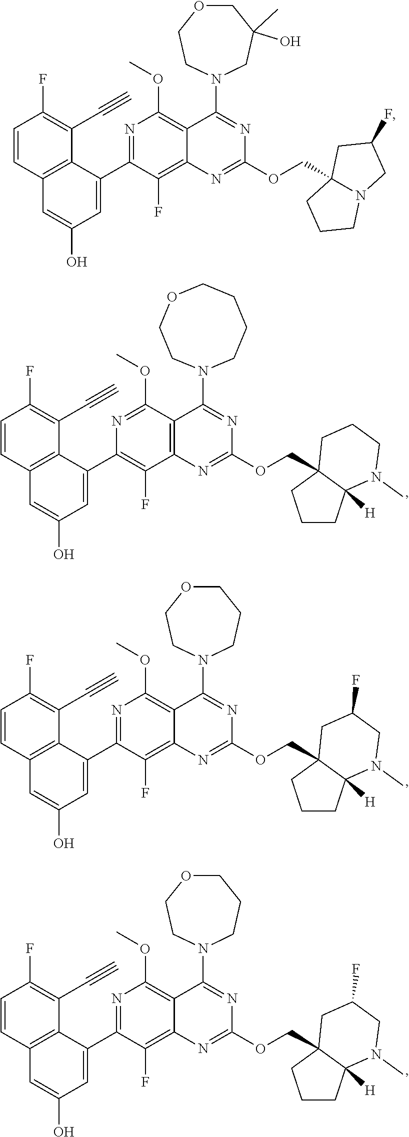 [SMILES: C#Cc1c(F)ccc2cc(O)cc(-c3nc(OC)c4c(N5CCOCC(C)(O)C5)nc(OC[C@@]56CCCN5C[C@H](F)C6)nc4c3F)c12 ; C#Cc1c(F)ccc2cc(O)cc(-c3nc(OC)c4c(N5CCCCOCC5)nc(OC[C@]56CCC[C@H]5N(C)CCC6)nc4c3F)c12 ; C#Cc1c(F)ccc2cc(O)cc(-c3nc(OC)c4c(N5CCCOCC5)nc(OC[C@]56CCC[C@H]5N(C)C[C@H](F)C6)nc4c3F)c12 ; C#Cc1c(F)ccc2cc(O)cc(-c3nc(OC)c4c(N5CCCOCC5)nc(OC[C@]56CCC[C@H]5N(C)C[C@@H](F)C6)nc4c3F)c12]

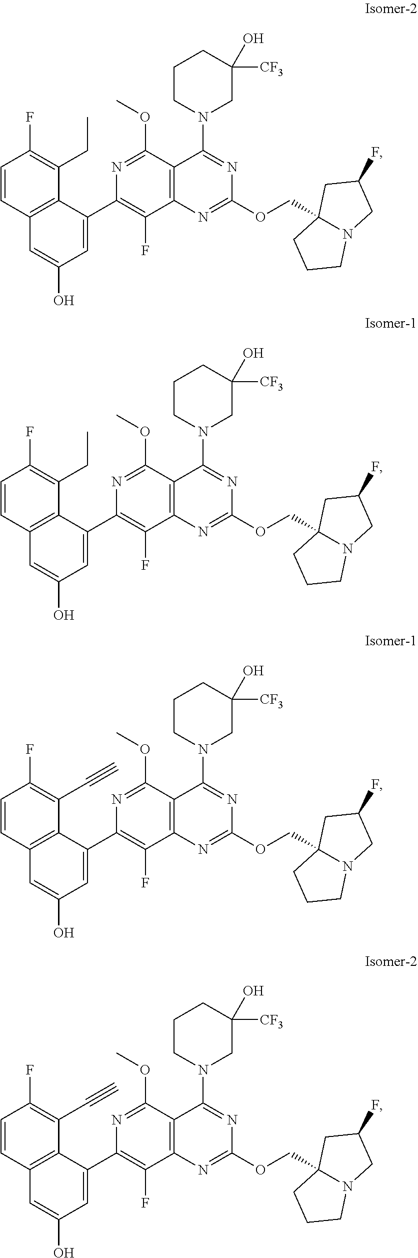 [SMILES: CCc1c(F)ccc2cc(O)cc(-c3nc(OC)c4c(N5CCCC(O)(C(F)(F)F)C5)nc(OC[C@@]56CCCN5C[C@H](F)C6)nc4c3F)c12 ; CCc1c(F)ccc2cc(O)cc(-c3nc(OC)c4c(N5CCCC(O)(C(F)(F)F)C5)nc(OC[C@@]56CCCN5C[C@H](F)C6)nc4c3F)c12 ; C#Cc1c(F)ccc2cc(O)cc(-c3nc(OC)c4c(N5CCCC(O)(C(F)(F)F)C5)nc(OC[C@@]56CCCN5C[C@H](F)C6)nc4c3F)c12 ; C#Cc1c(F)ccc2cc(O)cc(-c3nc(OC)c4c(N5CCCC(O)(C(F)(F)F)C5)nc(OC[C@@]56CCCN5C[C@H](F)C6)nc4c3F)c12]

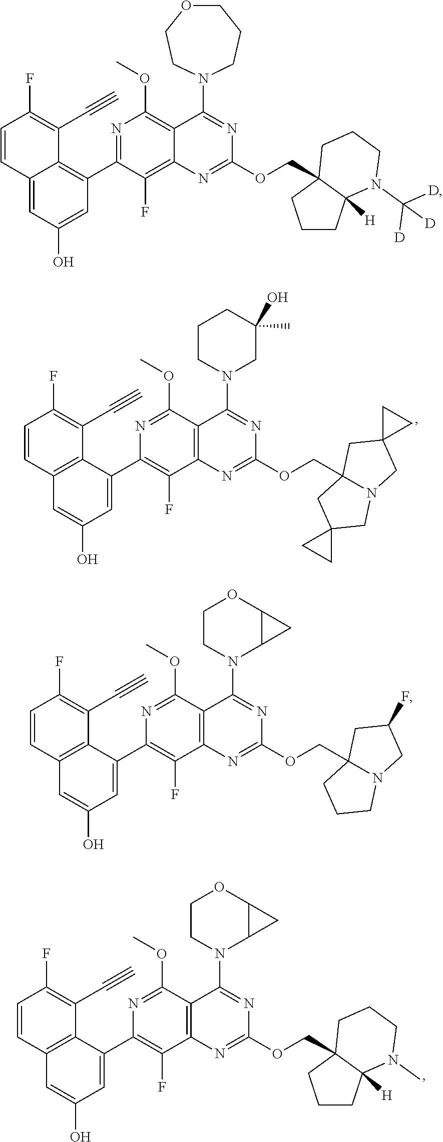

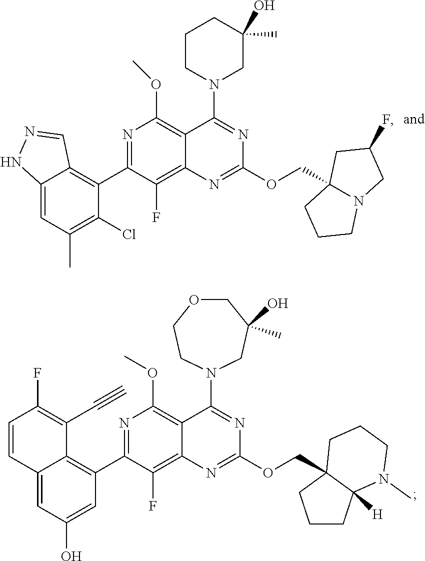 [SMILES: COc1nc(-c2c(Cl)c(C)cc3[nH]ncc23)c(F)c2nc(OC[C@@]34CCCN3C[C@H](F)C4)nc(N3CCC[C@@](C)(O)C3)c12 ; C#Cc1c(F)ccc2cc(O)cc(-c3nc(OC)c4c(N5CCOC[C@@](C)(O)C5)nc(OC[C@]56CCC[C@H]5N(C)CCC6)nc4c3F)c12]

or a pharmaceutically acceptable salt thereof.

In some aspects, the present disclosure provides a compound selected from the group consisting of:
4-(2-{[(2R,7aS)-2-fluoro-hexahydro-1H-pyrrolizin-7a-yl]methoxy}-8-fluoro-4-(morpholin-4-yl)pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-(1,4-oxazepan-4-yl)pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-4-(1,4-oxazepan-4-yl)pyrido[4,3-d]pyrimidin-7-yl)-5-ethynyl-6-fluoronaphthalen-2-ol;
5-ethynyl-6-fluoro-4-{8-fluoro-2-[(1-{[(3R)-3-fluoropyrrolidin-1-yl]methyl}cyclopropyl)methoxy]-4-(morpholin-4-yl)pyrido[4,3-d]pyrimidin-7-yl}naphthalen-2-ol;
4-[2-({1-[(dimethylamino)methyl]cyclopropyl}methoxy)-8-fluoro-4-(morpholin-4-yl)pyrido[4,3-d]pyrimidin-7-yl]-5-ethynyl-6-fluoronaphthalen-2-ol;
5-ethynyl-6-fluoro-4-[8-fluoro-4-(morpholin-4-yl)-2-({1-[(piperidin-1-yl)methyl]cyclopropyl}methoxy)pyrido[4,3-d]pyrimidin-7-yl]naphthalen-2-ol;
4-(2-{[(2R,7aS)-2-fluoro-hexahydro-1H-pyrrolizin-7a-yl]methoxy}-8-fluoro-4-{8-oxa-3-azabicyclo[3.2.1]octan-3-yl}pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-4-(morpholin-4-yl)pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-4-{8-oxa-3-azabicyclo[3.2.1]octan-3-yl}pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-4-[(2R,6S)-2,6-dimethylmorpholin-4-yl]-8-fluoropyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(2S)-2-methylmorpholin-4-yl]pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-4-[(2R,6S)-2,6-dimethylmorpholin-4-yl]-8-fluoropyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-4-[(3R,4S)-3,4-difluoropyrrolidin-1-yl]-8-fluoropyrido[4,3-d]pyrimidin-7-yl)-5-ethynyl-6-fluoronaphthalen-2-ol;
6-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-2¿6-thia-6-azaspiro[3.3]heptane-2,2-dione;
4-[2-({1-[(dimethylamino)methyl]cyclopropyl}methoxy)-8-fluoro-4-(morpholin-4-yl)pyrido[4,3-d]pyrimidin-7-yl]-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(3R)-3-(2-hydroxypropan-2-yl)pyrrolidin-1-yl]pyrido[4,3-d]pyrimidin-7-yl)-5-ethynyl-6-fluoronaphthalen-2-ol;
(8aR)-7-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-hexahydro-1H-[1,3]oxazolo[3,4-a]piperazin-3-one;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-N,N-dimethylpiperidine-4-sulfonamide;
4-(2-{[(2R,7aS)-2-fluoro-hexahydro-1H-pyrrolizin-7a-yl]methoxy}-8-fluoro-4-(4-methanesulfonylpiperidin-1-yl)pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-4-[(2R,6S)-2,6-dimethylmorpholin-4-yl]-8-fluoropyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-4-{2,2-difluoro-7-azaspiro[3.5]nonan-7-yl}-8-fluoropyrido[4,3-d]pyrimidin-7-yl)-5-ethynyl-6-fluoronaphthalen-2-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)piperidine-3-carbonitrile;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)piperidine-3-carbonitrile;
4-(2-{[(2R,7aS)-2-fluoro-hexahydro-1H-pyrrolizin-7a-yl]methoxy}-8-fluoro-4-[2-(hydroxymethyl)morpholin-4-yl]pyrido[4,3-d]pyrimidin-7-yl)-5-ethynyl-6-fluoronaphthalen-2-ol;
1-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoropyrido[4,3-d]pyrimidin-4-yl)piperidine-3-carbonitrile isomer 1;
1-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoropyrido[4,3-d]pyrimidin-4-yl)piperidine-3-carbonitrile isomer 2;
4-(2-{[(2R,7aS)-2-fluoro-hexahydro-1H-pyrrolizin-7a-yl]methoxy}-8-fluoro-4-[(3R)-3-methylmorpholin-4-yl]pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-4-[(3R)-3-methylmorpholin-4-yl]pyrido[4,3-d]pyrimidin-7-yl)-5-ethynyl-6-fluoronaphthalen-2-ol;
5-ethynyl-6-fluoro-4-[8-fluoro-2-({1-[(4-fluoropiperidin-1-yl)methyl]cyclopropyl}methoxy)-4-[(3R)-3-methylmorpholin-4-yl]pyrido[4,3-d]pyrimidin-7-yl]naphthalen-2-ol;
4-(2-{[(2R,7aS)-2-fluoro-hexahydro-1H-pyrrolizin-7a-yl]methoxy}-8-fluoro-4-[(2R)-2-(hydroxymethyl)morpholin-4-yl]pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(2S)-2-(hydroxymethyl)morpholin-4-yl]pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(3S)-3-methylmorpholin-4-yl]pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-4-[(3R)-3-ethylmorpholin-4-yl]-8-fluoropyrido[4,3-d]pyrimidin-7-yl)-5-ethynyl-6-fluoronaphthalen-2-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)piperidine-4-carbonitrile;
4-(2-{[(2R,7aS)-2-fluoro-hexahydro-1H-pyrrolizin-7a-yl]methoxy}-8-fluoro-4-{3-oxa-6-azabicyclo[3.1.1]heptan-6-yl}pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(1S,4S)-2-oxa-5-azabicyclo[2.2.1]heptan-5-yl]pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(1R,4R)-2-oxa-5-azabicyclo[2.2.1]heptan-5-yl]pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-{2-oxa-5-azabicyclo[4.1.0]heptan-5-yl}pyrido[4,3-d]pyrimidin-7-yl)-5-ethynyl-6-fluoronaphthalen-2-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-4-methylpiperidin-4-ol;
5-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-2-methyl-1¿6,2,5-thiadiazepane-1,1-dione;
4-(2-{[(2R,7aS)-2-fluoro-hexahydro-1H-pyrrolizin-7a-yl]methoxy}-8-fluoro-4-{9-oxa-2-azaspiro[5.5]undecan-2-yl}pyrido[4,3-d]pyrimidin-7-yl)-5-ethynyl-6-fluoronaphthalen-2-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)piperidin-3-ol isomer 1;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)piperidin-3-ol isomer 2;
1-[7-(8-ethynyl-7-fluoro-3-hydroxynaphthalen-1-yl)-8-fluoro-2-({1-[(4-fluoropiperidin-1-yl)methyl]cyclopropyl}methoxy)pyrido[4,3-d]pyrimidin-4-yl]piperidine-3-carbonitrile isomer 1;
1-[7-(8-ethynyl-7-fluoro-3-hydroxynaphthalen-1-yl)-8-fluoro-2-({1-[(4-fluoropiperidin-1-yl)methyl]cyclopropyl}methoxy)pyrido[4,3-d]pyrimidin-4-yl]piperidine-3-carbonitrile isomer 2;
7-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-7-azaspiro[3.5]nonan-2-ol;
4-(2-{[(2R,7aS)-2-fluoro-hexahydro-1H-pyrrolizin-7a-yl]methoxy}-8-fluoro-4-{hexahydro-1H-furo[3,4-c]pyrrol-5-yl}pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-4-{8-azabicyclo[3.2.1]octan-8-yl}-8-fluoropyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-4-{2-azabicyclo[2.2.1]heptan-2-yl}-8-fluoropyrido[4,3-d]pyrimidin-7-yl)-5-ethynyl-6-fluoronaphthalen-2-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)piperidin-4-ol;
4-(2-{[(2R,7aS)-2-fluoro-hexahydro-1H-pyrrolizin-7a-yl]methoxy}-8-fluoro-4-(4-methoxypiperidin-1-yl)pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[3-(hydroxymethyl)piperidin-1-yl]pyrido[4,3-d]pyrimidin-7-yl)-5-ethynyl-6-fluoronaphthalen-2-ol;
3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-3-azabicyclo[3.2.1]octan-8-ol;
3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-8-methyl-3-azabicyclo[3.2.1]octan-8-ol;
(3aR,5R,6aS)-2-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-5-(difluoromethyl)-octahydrocyclopenta[c]pyrrol-5-ol;
(1R,5S,6R)-3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-6-methyl-3-azabicyclo[3.1.1]heptan-6-ol;
2-[1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)piperidin-3-yl]acetonitrile;
3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-3-azabicyclo[3.2.1]octan-6-ol;
3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-3-azabicyclo[3.3.1]nonan-9-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-4-hydroxypiperidine-3-carbonitrile isomer 1;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-4-hydroxypiperidine-3-carbonitrile isomer 2;
4-(2-{[(2R,7aS)-2-fluoro-hexahydro-1H-pyrrolizin-7a-yl]methoxy}-8-fluoro-4-[3-(hydroxymethyl)piperidin-1-yl]pyrido[4,3-d]pyrimidin-7-yl)-5-ethynyl-6-fluoronaphthalen-2-ol isomer 1;
4-(2-{[(2R,7aS)-2-fluoro-hexahydro-1H-pyrrolizin-7a-yl]methoxy}-8-fluoro-4-[3-(hydroxymethyl)piperidin-1-yl]pyrido[4,3-d]pyrimidin-7-yl)-5-ethynyl-6-fluoronaphthalen-2-ol isomer 2;
4-(2-{[(2R,7aS)-2-fluoro-hexahydro-1H-pyrrolizin-7a-yl]methoxy}-8-fluoro-4-[(3R)-3-(methoxymethyl)morpholin-4-yl]pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(3S)-3-(methoxymethyl)morpholin-4-yl]pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-4-[4-(dimethylphosphoryl)piperidin-1-yl]-8-fluoropyrido[4,3-d]pyrimidin-7-yl)-5-ethynyl-6-fluoronaphthalen-2-ol;
(3aR,5S,6aS)-2-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-5-methyl-octahydrocyclopenta[c]pyrrol-5-ol;
4-(2-{[(2R,7aS)-2-fluoro-hexahydro-1H-pyrrolizin-7a-yl]methoxy}-8-fluoro-4-[4-(methoxymethyl)piperidin-1-yl]pyrido[4,3-d]pyrimidin-7-yl)-5-ethynyl-6-fluoronaphthalen-2-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-3-methylpiperidine-3-carbonitrile isomer 1;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-3-methylpiperidine-3-carbonitrile isomer 2;
4-(2-{[(2R,7aS)-2-fluoro-hexahydro-1H-pyrrolizin-7a-yl]methoxy}-4-[3-(dimethylphosphoryl)piperidin-1-yl]-8-fluoropyrido[4,3-d]pyrimidin-7-yl)-5-ethynyl-6-fluoronaphthalen-2-ol isomer 1;
4-(2-{[(2R,7aS)-2-fluoro-hexahydro-1H-pyrrolizin-7a-yl]methoxy}-4-[3-(dimethylphosphoryl)piperidin-1-yl]-8-fluoropyrido[4,3-d]pyrimidin-7-yl)-5-ethynyl-6-fluoronaphthalen-2-ol isomer 2;
4-(2-{[(2R,7aS)-2-fluoro-hexahydro-1H-pyrrolizin-7a-yl]methoxy}-8-fluoro-4-[2-(methoxymethyl)piperidin-1-yl]pyrido[4,3-d]pyrimidin-7-yl)-5-ethynyl-6-fluoronaphthalen-2-ol isomer 1;
4-(2-{[(2R,7aS)-2-fluoro-hexahydro-1H-pyrrolizin-7a-yl]methoxy}-8-fluoro-4-[2-(methoxymethyl)piperidin-1-yl]pyrido[4,3-d]pyrimidin-7-yl)-5-ethynyl-6-fluoronaphthalen-2-ol isomer 2;
4-(2-{[(4aS,7aR)-1-methyl-octahydro-1H-cyclopenta[b]pyridin-4a-yl]methoxy}-8-fluoro-4-{2-oxa-5-azabicyclo[4.1.0]heptan-5-yl}pyrido[4,3-d]pyrimidin-7-yl)-5-ethynyl-6-fluoronaphthalen-2-ol;
1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-4-methyl-1,4-azaphosphinane 4-oxide;
4-(2-{[(4aS,7aR)-1-methyl-octahydro-1H-cyclopenta[b]pyridin-4a-yl]methoxy}-8-fluoro-4-{3-oxa-6-azabicyclo[3.1.1]heptan-6-yl}pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-4-[(1S,4S)-2-oxa-5-azabicyclo[2.2.1]heptan-5-yl]pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-4-(6-methyl-1,4-oxazepan-4-yl)pyrido[4,3-d]pyrimidin-7-yl)-5-ethynyl-6-fluoronaphthalen-2-ol;
1-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoropyrido[4,3-d]pyrimidin-4-yl)-3-methylpiperidin-3-ol;
4-(2-{[(4aS,7aR)-1-methyl-octahydro-1H-cyclopenta[b]pyridin-4a-yl]methoxy}-8-fluoro-4-[6-(hydroxymethyl)-1,4-oxazepan-4-yl]pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-4-[6-(hydroxymethyl)-1,4-oxazepan-4-yl]pyrido[4,3-d]pyrimidin-7-yl)-5-ethyl-6-fluoronaphthalen-2-ol;
3-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoropyrido[4,3-d]pyrimidin-4-yl)-3-azabicyclo[3.2.1]octan-6-ol;
4-(2-{[(2R,7aS)-2-fluoro-hexahydro-1H-pyrrolizin-7a-yl]methoxy}-8-fluoro-4-(morpholin-4-yl)quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-(1,4-oxazepan-4-yl)quinazolin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-4-(1,4-oxazepan-4-yl)quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-{8-oxa-3-azabicyclo[3.2.1]octan-3-yl}quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(2S)-2-methylmorpholin-4-yl]quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(3R)-3-methylmorpholin-4-yl]quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-4-[(2R,6S)-2,6-dimethylmorpholin-4-yl]-8-fluoroquinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[2-(hydroxymethyl)morpholin-4-yl]quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(3S)-3-methylmorpholin-4-yl]quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-(pyrrolidin-1-yl)quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-4-{6,6-dimethyl-3-azabicyclo[3.1.0]hexan-3-yl}-8-fluoroquinazolin-7-yl)-5-ethynyl-6-fluoronaphthalen-2-ol;
4-(2-{[(2R,7aS)-2-fluoro-hexahydro-1H-pyrrolizin-7a-yl]methoxy}-4-[(3R,4S)-3,4-difluoropyrrolidin-1-yl]-8-fluoroquinazolin-7-yl)-5-ethynyl-6-fluoronaphthalen-2-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)piperidine-3-carbonitrile;
4-(2-{[(2R,7aS)-2-fluoro-hexahydro-1H-pyrrolizin-7a-yl]methoxy}-8-fluoro-4-[(3R)-3-(2-hydroxypropan-2-yl)pyrrolidin-1-yl]quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-1¿6-thiomorpholine-1,1-dione;
(9aR)-8-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-octahydropiperazino[2,1-c]morpholin-4-one;
(8aR)-7-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-hexahydro-1H-[1,3]oxazolo[3,4-a]piperazin-3-one;
4-(2-{[(2R,7aS)-2-fluoro-hexahydro-1H-pyrrolizin-7a-yl]methoxy}-8-fluoro-4-(4-methanesulfonylpiperidin-1-yl)quinazolin-7-yl)-5-ethynyl-6-fluoronaphthalen-2-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-N,N-dimethylpiperidine-4-sulfonamide;
4-(2-{[(2R,7aS)-2-fluoro-hexahydro-1H-pyrrolizin-7a-yl]methoxy}-8-fluoro-4-{2-oxa-7-azaspiro[3.5]nonan-7-yl}quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-4-{2,2-difluoro-7-azaspiro[3.5]nonan-7-yl}-8-fluoroquinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-(piperidin-1-yl)quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-4-{2-azabicyclo[2.2.1]heptan-2-yl}-8-fluoroquinazolin-7-yl)-5-ethynyl-6-fluoronaphthalen-2-ol;
4-(2-{[(2R,7aS)-2-fluoro-hexahydro-1H-pyrrolizin-7a-yl]methoxy}-4-{8-azabicyclo[3.2.1]octan-8-yl}-8-fluoroquinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-{hexahydro-1H-furo[3,4-c]pyrrol-5-yl}quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(1S,4S)-2-oxa-5-azabicyclo[2.2.1]heptan-5-yl]quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-{3-oxa-6-azabicyclo[3.1.1]heptan-6-yl}quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(1R,4R)-2-oxa-5-azabicyclo[2.2.1]heptan-5-yl]quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(2R)-2-(hydroxymethyl)morpholin-4-yl]quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(2S)-2-(hydroxymethyl)morpholin-4-yl]quinazolin-7-yl)-5-ethynyl-6-fluoronaphthalen-2-ol;
(8aS)-7-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-hexahydro-1H-[1,3]oxazolo[3,4-a]piperazin-3-one;
4-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)morpholine-2-carbonitrile;
4-(2-{[(2R,7aS)-2-fluoro-hexahydro-1H-pyrrolizin-7a-yl]methoxy}-4-[(3R)-3-ethylmorpholin-4-yl]-8-fluoroquinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-{2-oxa-5-azabicyclo[4.1.0]heptan-5-yl}quinazolin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-4-[(3R)-3-methylmorpholin-4-yl]quinazolin-7-yl)-5-ethynyl-6-fluoronaphthalen-2-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)piperidine-4-carbonitrile;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)piperidin-3-ol;
7-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-7-azaspiro[3.5]nonan-2-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-4-methylpiperidin-4-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)piperidine-3-carbonitrile isomer 1;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)piperidine-3-carbonitrile isomer 2;
5-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-2-methyl-1¿6,2,5-thiadiazepane-1,1-dione;
4-(2-{[(2R,7aS)-2-fluoro-hexahydro-1H-pyrrolizin-7a-yl]methoxy}-4-[(3S)-3-ethylmorpholin-4-yl]-8-fluoroquinazolin-7-yl)-5-ethynyl-6-fluoronaphthalen-2-ol;
1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)piperidin-4-ol;
4-(2-{[(2R,7aS)-2-fluoro-hexahydro-1H-pyrrolizin-7a-yl]methoxy}-8-fluoro-4-(4-methoxypiperidin-1-yl)quinazolin-7-yl)-5-ethynyl-6-fluoronaphthalen-2-ol;
3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-3-azabicyclo[3.2.1]octan-8-ol;
3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-8-methyl-3-azabicyclo[3.2.1]octan-8-ol;
(8aS)-7-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoroquinazolin-4-yl)-hexahydro-1H-[1,3]oxazolo[3,4-a]piperazin-3-one;
3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-3-azabicyclo[3.3.1]nonan-9-ol;
(3aR,5R,6aS)-2-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-5-(difluoromethyl)-octahydrocyclopenta[c]pyrrol-5-ol;
4-(2-{[(2R,7aS)-2-fluoro-hexahydro-1H-pyrrolizin-7a-yl]methoxy}-8-fluoro-4-[(3R)-3-(methoxymethyl)morpholin-4-yl]quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[(3S)-3-(methoxymethyl)morpholin-4-yl]quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-4-[4-(dimethylphosphoryl)piperidin-1-yl]-8-fluoroquinazolin-7-yl)-5-ethynyl-6-fluoronaphthalen-2-ol;
(3aR,5S,6aS)-2-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-5-methyl-octahydrocyclopenta[c]pyrrol-5-ol;
4-(2-{[(2R,7aS)-2-fluoro-hexahydro-1H-pyrrolizin-7a-yl]methoxy}-8-fluoro-4-[4-(methoxymethyl)piperidin-1-yl]quinazolin-7-yl)-5-ethynyl-6-fluoronaphthalen-2-ol;
3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-3-azabicyclo[3.1.1]heptan-6-ol;
4-(2-{[(2R,7aS)-2-fluoro-hexahydro-1H-pyrrolizin-7a-yl]methoxy}-8-fluoro-4-{9-oxa-3-azabicyclo[4.2.1]nonan-3-yl}quinazolin-7-yl)-5-ethynyl-6-fluoronaphthalen-2-ol;
(3R)-1-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoroquinazolin-4-yl)piperidin-3-ol;
(3R)-1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)piperidin-3-ol;
4-(2-{[(2R,7aS)-2-fluoro-hexahydro-1H-pyrrolizin-7a-yl]methoxy}-8-fluoro-4-(6-methyl-1,4-oxazepan-4-yl)quinazolin-7-yl)-5-ethynyl-6-fluoronaphthalen-2-ol;
(3S)-1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)piperidin-3-ol;
4-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoroquinazolin-4-yl)-6-methyl-1,4-oxazepan-6-ol;
4-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-6-methyl-1,4-oxazepan-6-ol;
4-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-1,4-oxazepan-6-ol;
4-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoroquinazolin-4-yl)-1,4-oxazepan-6-ol;
4-(2-{[(2R,7aS)-2-fluoro-hexahydro-1H-pyrrolizin-7a-yl]methoxy}-8-fluoro-4-(6-methoxy-1,4-oxazepan-4-yl)quinazolin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-4-(6-methoxy-1,4-oxazepan-4-yl)quinazolin-7-yl)-5-ethynyl-6-fluoronaphthalen-2-ol;
6-(2-{[(4aS,7aR)-1-methyl-octahydro-1H-cyclopenta[b]pyridin-4a-yl]methoxy}-6-chloro-8-fluoro-4-{8-oxa-3-azabicyclo[3.2.1]octan-3-yl}quinazolin-7-yl)-4-methyl-5-(trifluoromethyl)pyridin-2-amine;
4-(2-{[(4aS,7aR)-1-methyl-octahydro-1H-cyclopenta[b]pyridin-4a-yl]methoxy}-8-fluoro-4-(morpholin-4-yl)quinazolin-7-yl)-5-ethylnaphthalen-2-ol;
4-(2-{[(4aS,7aR)-1-methyl-octahydro-1H-cyclopenta[b]pyridin-4a-yl]methoxy}-8-fluoro-4-(morpholin-4-yl)quinazolin-7-yl)-5-ethylnaphthalen-2-ol;
(1S,4S)-5-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoroquinazolin-4-yl)-2,5-diazabicyclo[2.2.1]heptan-3-one;
1-[3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-3,8-diazabicyclo[3.2.1]octan-8-yl]-2,2,2-trifluoroethan-1-one;
4-(2-{[(2R,7aS)-2-fluoro-hexahydro-1H-pyrrolizin-7a-yl]methoxy}-4-{8-cyclopropanecarbonyl-3,8-diazabicyclo[3.2.1]octan-3-yl}-8-fluoropyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[8-(1-fluorocyclopropanecarbonyl)-3,8-diazabicyclo[3.2.1]octan-3-yl]pyrido[4,3-d]pyrimidin-7-yl)-5-ethynyl-6-fluoronaphthalen-2-ol;
1-[3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-3,8-diazabicyclo[3.2.1]octane-8-carbonyl]cyclopropane-1-carbonitrile;
4-(2-{[(2R,7aS)-2-fluoro-hexahydro-1H-pyrrolizin-7a-yl]methoxy}-8-fluoro-4-[8-(2-fluorocyclopropanecarbonyl)-3,8-diazabicyclo[3.2.1]octan-3-yl]pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-4-[8-(2,2-difluorocyclopropanecarbonyl)-3,8-diazabicyclo[3.2.1]octan-3-yl]-8-fluoropyrido[4,3-d]pyrimidin-7-yl)-5-ethynyl-6-fluoronaphthalen-2-ol isomer 1;
4-(2-{[(2R,7aS)-2-fluoro-hexahydro-1H-pyrrolizin-7a-yl]methoxy}-4-[8-(2,2-difluorocyclopropanecarbonyl)-3,8-diazabicyclo[3.2.1]octan-3-yl]-8-fluoropyrido[4,3-d]pyrimidin-7-yl)-5-ethynyl-6-fluoronaphthalen-2-ol isomer 2;
4-(2-{[(4aS,7aR)-1-methyl-octahydro-1H-cyclopenta[b]pyridin-4a-yl]methoxy}-4-[8-(3,3-difluorocyclobutanecarbonyl)-3,8-diazabicyclo[3.2.1]octan-3-yl]-8-fluoropyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-4-{8-cyclopropanecarbonyl-3,8-diazabicyclo[3.2.1]octan-3-yl}-8-fluoropyrido[4,3-d]pyrimidin-7-yl)-5-ethynyl-6-fluoronaphthalen-2-ol;
4-(4-{8-cyclopropanecarbonyl-3,8-diazabicyclo[3.2.1]octan-3-yl}-8-fluoro-2-({1-[(4-fluoropiperidin-1-yl)methyl]cyclopropyl}methoxy)pyrido[4,3-d]pyrimidin-7-yl)-5-ethynyl-6-fluoronaphthalen-2-ol;
1-[3-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoropyrido[4,3-d]pyrimidin-4-yl)-3,8-diazabicyclo[3.2.1]octan-8-yl]-2,2,2-trifluoroethan-1-one;
4-(2-{[(2R,7aS)-2-fluoro-hexahydro-1H-pyrrolizin-7a-yl]methoxy}-4-[8-(4,4-difluorocyclohexanecarbonyl)-3,8-diazabicyclo[3.2.1]octan-3-yl]-8-fluoroquinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-[8-(oxane-4-carbonyl)-3,8-diazabicyclo[3.2.1]octan-3-yl]quinazolin-7-yl)-5-ethynyl-6-fluoronaphthalen-2-ol;
1-[3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-3,8-diazabicyclo[3.2.1]octan-8-yl]-2,2,2-trifluoroethan-1-one;
4-(2-{[(2R,7aS)-2-fluoro-hexahydro-1H-pyrrolizin-7a-yl]methoxy}-4-[8-(3,3-difluorocyclobutanecarbonyl)-3,8-diazabicyclo[3.2.1]octan-3-yl]-8-fluoroquinazolin-7-yl)-5-ethynyl-6-fluoronaphthalen-2-ol;
4-(2-{[(2R,7aS)-2-fluoro-hexahydro-1H-pyrrolizin-7a-yl]methoxy}-4-{8-cyclopropanecarbonyl-3,8-diazabicyclo[3.2.1]octan-3-yl}-8-fluoroquinazolin-7-yl)-5-ethynyl-6-7-6fluoronaphthalen-2-ol;
4-(2-{[(2R,7aS)-2-fluoro-hexahydro-1H-pyrrolizin-7a-yl]methoxy}-8-fluoro-4-{8-[(1s,4s)-4-hydroxycyclohexanecarbonyl]-3,8-diazabicyclo[3.2.1]octan-3-yl}quinazolin-7-yl)-5-ethynyl-6-fluoronaphthalen-2-ol;
1-[3-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoroquinazolin-4-yl)-3,8-diazabicyclo[3.2.1]octan-8-yl]-2,2,2-trifluoroethan-1-one;
4-(2-{[(4aS,7aR)-1-methyl-octahydro-1H-cyclopenta[b]pyridin-4a-yl]methoxy}-4-{8-cyclopropanecarbonyl-3,8-diazabicyclo[3.2.1]octan-3-yl}-8-fluoroquinazolin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-{8-methanesulfonyl-3,8-diazabicyclo[3.2.1]octan-3-yl}quinazolin-7-yl)-5-ethynyl-6-fluoronaphthalen-2-ol;
6-(2-{[(4aS,7aR)-1-methyl-octahydro-1H-cyclopenta[b]pyridin-4a-yl]methoxy}-6-chloro-4-[8-(3,3-difluorocyclobutanecarbonyl)-3,8-diazabicyclo[3.2.1]octan-3-yl]-8-fluoroquinazolin-7-yl)-4-methyl-5-(trifluoromethyl)pyridin-2-amine;
1-[3-(2-{[(4aS,7aR)-1-methyl-octahydro-1H-cyclopenta[b]pyridin-4a-yl]methoxy}-7-[6-amino-4-methyl-3-(trifluoromethyl)pyridin-2-yl]-6-chloro-8-fluoroquinazolin-4-yl)-3,8-diazabicyclo[3.2.1]octan-8-yl]-2,2,2-trifluoroethan-1-one;
4-(2-{[(6′R,7′aR)-6′-fluoro-hexahydrospiro[cyclopropane-1,2′-pyrrolizine]-7′a-yl]methoxy}-7-(8-ethynyl-7-fluoro-3-hydroxynaphthalen-1-yl)-8-fluoropyrido[4,3-d]pyrimidin-4-yl)-6-methyl-1,4-oxazepan-6-ol;
1-(2-{[(4aS,7aR)-1-methyl-octahydro-1H-cyclopenta[b]pyridin-4a-yl]methoxy}-7-(8-ethyl-7-fluoro-3-hydroxynaphthalen-1-yl)-8-fluoropyrido[4,3-d]pyrimidin-4-yl)-3-methylpiperidin-3-ol;
3-(2-{[(4aS,7aR)-1-methyl-octahydro-1H-cyclopenta[b]pyridin-4a-yl]methoxy}-7-(8-ethyl-7-fluoro-3-hydroxynaphthalen-1-yl)-8-fluoropyrido[4,3-d]pyrimidin-4-yl)-3-azabicyclo[3.2.1]octan-6-ol;
(3R)-1-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoropyrido[4,3-d]pyrimidin-4-yl)piperidin-3-ol;
4-(2-{[(4aS,7aR)-1-methyl-octahydro-1H-cyclopenta[b]pyridin-4a-yl]methoxy}-8-fluoro-4-[6-(hydroxymethyl)-1,4-oxazepan-4-yl]pyrido[4,3-d]pyrimidin-7-yl)-5-ethynyl-6-fluoronaphthalen-2-ol;
1-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoropyrido[4,3-d]pyrimidin-4-yl)-3-methylpiperidin-3-ol;
4-(2-{[(2R,7aS)-2-fluoro-hexahydro-1H-pyrrolizin-7a-yl]methoxy}-8-fluoro-4-{6-oxa-3-azabicyclo[3.2.1]octan-3-yl}pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-(6-methyl-1,4-oxazepan-4-yl)pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-4-(1,4-oxazepan-4-yl)pyrido[4,3-d]pyrimidin-7-yl)-5-ethyl-6-fluoronaphthalen-2-ol;
4-(2-{[(4aS,7aR)-1-methyl-octahydro-1H-cyclopenta[b]pyridin-4a-yl]methoxy}-8-fluoro-4-(5-methyl-1,4-oxazepan-4-yl)pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-4-(azepan-1-yl)-8-fluoropyrido[4,3-d]pyrimidin-7-yl)-5-ethynyl-6-fluoronaphthalen-2-ol;
1-(2-{[(6′R,7′aR)-6′-fluoro-hexahydrospiro[cyclopropane-1,2′-pyrrolizine]-7′a-yl]methoxy}-7-(8-ethynyl-7-fluoro-3-hydroxynaphthalen-1-yl)-8-fluoropyrido[4,3-d]pyrimidin-4-yl)-3-methylpiperidin-3-ol;
4-(2-{[(2R,7aS)-2-fluoro-hexahydro-1H-pyrrolizin-7a-yl]methoxy}-8-fluoro-4-(5-methyl-1,4-oxazepan-4-yl)pyrido[4,3-d]pyrimidin-7-yl)-5-ethynyl-6-fluoronaphthalen-2-ol;
(1S,4S)-5-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-2,5-diazabicyclo[2.2.1]heptan-3-one;
4-(2-{[(4aS,7aR)-1-methyl-octahydro-1H-cyclopenta[b]pyridin-4a-yl]methoxy}-8-fluoro-4-(1,4-oxazepan-4-yl)pyrido[4,3-d]pyrimidin-7-yl)-5-chloronaphthalen-2-ol;
4-(2-{[(4aS,7aR)-1-methyl-octahydro-1H-cyclopenta[b]pyridin-4a-yl]methoxy}-8-fluoro-4-{2-oxa-5-azabicyclo[4.1.0]heptan-5-yl}pyrido[4,3-d]pyrimidin-7-yl)-5-ethynyl-6-fluoronaphthalen-2-ol;
4-(2-{[(2R,7aS)-2-fluoro-hexahydro-1H-pyrrolizin-7a-yl]methoxy}-8-fluoro-4-{2-oxa-5-azabicyclo[4.1.0]heptan-5-yl}pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-4-{1-methyl-1H,4H,5H,6H-pyrrolo[3,4-c]pyrazol-5-yl}pyrido[4,3-d]pyrimidin-7-yl)-5-ethynyl-6-fluoronaphthalen-2-ol;
5-ethynyl-6-fluoro-4-{8-fluoro-2-[(1-methyl-octahydro-1H-indol-3a-yl)methoxy]-4-(1,4-oxazepan-4-yl)pyrido[4,3-d]pyrimidin-7-yl}naphthalen-2-ol;
4-(2-{[(4aS,7aR)-1-methyl-octahydro-1H-cyclopenta[b]pyridin-4a-yl]methoxy}-8-fluoro-4-{2-methyl-2H,4H,5H,6H-pyrrolo[3,4-c]pyrazol-5-yl}pyrido[4,3-d]pyrimidin-7-yl)-5-ethynyl-6-fluoronaphthalen-2-ol;
3-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoropyrido[4,3-d]pyrimidin-4-yl)-3-azabicyclo[3.2.1]octan-6-ol isomer 1;
3-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoropyrido[4,3-d]pyrimidin-4-yl)-3-azabicyclo[3.2.1]octan-6-ol isomer 2;
4-(2-{[(6′R,7′aR)-6′-fluoro-hexahydrospiro[cyclopropane-1,2′-pyrrolizine]-7′a-yl]methoxy}-8-fluoro-4-(1,4-oxazepan-4-yl)pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-4-(6,6-difluoro-1,4-oxazepan-4-yl)-8-fluoropyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoropyrido[4,3-d]pyrimidin-4-yl)-6-methyl-1,4-oxazepan-6-ol;
(4R)-4-[(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoroquinazolin-4-yl)amino]pyrrolidin-2-one;
4-[2-({1H,2H,3H,5H,9bH-benzo[a]pyrrolizin-9b-yl}methoxy)-8-fluoro-4-(1,4-oxazepan-4-yl)quinazolin-7-yl]-5-ethynyl-6-fluoronaphthalen-2-ol;
4-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoroquinazolin-4-yl)-6-methyl-1,4-oxazepan-6-ol;
4-(2-{[(4aS,7aR)-1-methyl-octahydro-1H-cyclopenta[b]pyridin-4a-yl]methoxy}-8-fluoro-4-[(1R,4R)-2-oxa-5-azabicyclo[2.2.1]heptan-5-yl]quinazolin-7-yl)-5-ethynyl-6-fluoronaphthalen-2-ol;
1-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoroquinazolin-4-yl)-3-methylpiperidin-3-ol;
4-(2-{[(4aS,7aR)-1-methyl-octahydro-1H-cyclopenta[b]pyridin-4a-yl]methoxy}-8-fluoro-4-{2-methyl-2H,4H,5H,6H-pyrrolo[3,4-c]pyrazol-5-yl}quinazolin-7-yl)-5-ethynyl-6-fluoronaphthalen-2-ol;
4-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoroquinazolin-4-yl)-6-methyl-1,4-oxazepan-6-ol;
4-(2-{[(2R,7aS)-2-fluoro-hexahydro-1H-pyrrolizin-7a-yl]methoxy}-8-fluoro-5-methoxy-4-(1,4-oxazepan-4-yl)pyrido[4,3-d]pyrimidin-7-yl)-5-ethynyl-6-fluoronaphthalen-2-ol;
4-(2-{[(4aS,7aR)-1-methyl-octahydro-1H-cyclopenta[b]pyridin-4a-yl]methoxy}-8-fluoro-5-methoxy-4-(1,4-oxazepan-4-yl)pyrido[4,3-d]pyrimidin-7-yl)-5-ethynyl-6-fluoronaphthalen-2-ol;
(3R)-1-(2-{[(4aS,7aR)-1-methyl-octahydro-1H-cyclopenta[b]pyridin-4a-yl]methoxy}-8-fluoro-7-(3-hydroxynaphthalen-1-yl)pyrido[4,3-d]pyrimidin-4-yl)-3-methylpiperidin-3-ol;
(3R)-1-(2-{[(4aS,7aR)-1-methyl-octahydro-1H-cyclopenta[b]pyridin-4a-yl]methoxy}-8-fluoro-7-[5-hydroxy-2-(propan-2-yl)phenyl]pyrido[4,3-d]pyrimidin-4-yl)-3-methylpiperidin-3-ol;
(3R)-1-(2-{[(4aS,7aR)-1-methyl-octahydro-1H-cyclopenta[b]pyridin-4a-yl]methoxy}-7-(2-cyclobutyl-5-hydroxyphenyl)-8-fluoropyrido[4,3-d]pyrimidin-4-yl)-3-methylpiperidin-3-ol;
(3R)-1-(2-{[(4aS,7aR)-1-methyl-octahydro-1H-cyclopenta[b]pyridin-4a-yl]methoxy}-7-(2-{bicyclo[1.1.1]pentan-1-yl}-5-hydroxyphenyl)-8-fluoropyrido[4,3-d]pyrimidin-4-yl)-3-methylpiperidin-3-ol;
5-(2-{[(4aS,7aR)-1-methyl-octahydro-1H-cyclopenta[b]pyridin-4a-yl]methoxy}-8-fluoro-4-[(3R)-3-hydroxy-3-methylpiperidin-1-yl]pyrido[4,3-d]pyrimidin-7-yl)-1-(difluoromethyl)-1,2-dihydropyridin-2-one;
4-(2-{[(4aS,7aR)-1-methyl-octahydro-1H-cyclopenta[b]pyridin-4a-yl]methoxy}-8-fluoro-4-[(3R)-3-hydroxy-3-methylpiperidin-1-yl]pyrido[4,3-d]pyrimidin-7-yl)-2-(difluoromethyl)-1,2-dihydroisoquinolin-1-one;
(3R)-1-(2-{[(4aS,7aR)-1-methyl-octahydro-1H-cyclopenta[b]pyridin-4a-yl]methoxy}-7-[1-(difluoromethoxy)isoquinolin-4-yl]-8-fluoropyrido[4,3-d]pyrimidin-4-yl)-3-methylpiperidin-3-ol;
4-(2-{[(4aS,7aR)-1-methyl-octahydro-1H-cyclopenta[b]pyridin-4a-yl]methoxy}-8-fluoro-4-[(3R)-3-hydroxy-3-methylpiperidin-1-yl]pyrido[4,3-d]pyrimidin-7-yl)-2-(difluoromethyl)-6-(trifluoromethyl)-1,2-dihydroisoquinolin-1-one;
(3R)-1-(2-{[(4aS,7aR)-1-methyl-octahydro-1H-cyclopenta[b]pyridin-4a-yl]methoxy}-7-[1-(difluoromethoxy)-6-(trifluoromethyl)isoquinolin-4-yl]-8-fluoropyrido[4,3-d]pyrimidin-4-yl)-3-methylpiperidin-3-ol;
(3R)-1-(2-{[(4aS,7aR)-1-methyl-octahydro-1H-cyclopenta[b]pyridin-4a-yl]methoxy}-8-fluoro-7-(3-hydroxynaphthalen-1-yl)quinazolin-4-yl)-3-methylpiperidin-3-ol;
(3R)-1-(2-{[(4aS,7aR)-1-methyl-octahydro-1H-cyclopenta[b]pyridin-4a-yl]methoxy}-8-fluoro-7-[5-hydroxy-2-(propan-2-yl)phenyl]quinazolin-4-yl)-3-methylpiperidin-3-ol;
(3R)-1-(2-{[(4aS,7aR)-1-methyl-octahydro-1H-cyclopenta[b]pyridin-4a-yl]methoxy}-7-(2-cyclobutyl-5-hydroxyphenyl)-8-fluoroquinazolin-4-yl)-3-methylpiperidin-3-ol;
4-(4-(2-oxa-5-azabicyclo[4.1.0]heptan-5-yl)-8-fluoro-2-(((4aS,7aR)-1-methyloctahydro-4aH-cyclopenta[b]pyridin-4a-yl)methoxy)pyrido[4,3-d]pyrimidin-7-yl)-5-ethyl-6-fluoronaphthalen-2-ol;
1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)azepan-4-ol;
5-ethyl-6-fluoro-4-(8-fluoro-2-(((6′R,7a′R)-6′-fluorodihydro-1′H,3′H-spiro[cyclopropane-1,2′-pyrrolizin]-7a′(5′H)-yl)methoxy)-4-(1,4-oxazepan-4-yl)pyrido[4,3-d]pyrimidin-7-yl)naphthalen-2-ol;
5-ethynyl-6-fluoro-4-(8-fluoro-4-(2-methyl-1,4-oxazepan-4-yl)-2-(((4aS,7aR)-1-methyloctahydro-4aH-cyclopenta[b]pyridin-4a-yl)methoxy)pyrido[4,3-d]pyrimidin-7-yl)naphthalen-2-ol isomer 1;
5-ethynyl-6-fluoro-4-(8-fluoro-4-(2-methyl-1,4-oxazepan-4-yl)-2-(((4aS,7aR)-1-methyloctahydro-4aH-cyclopenta[b]pyridin-4a-yl)methoxy)pyrido[4,3-d]pyrimidin-7-yl)naphthalen-2-ol isomer 2;
4-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-1,4-oxazepan-6-ol;
1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-3-methylazepan-3-ol;
5-ethynyl-6-fluoro-4-(8-fluoro-4-(1,4-oxazepan-4-yl)-2-(((6′R,7a′R)-2,2,6′-trifluorodihydro-1′H,3′H-spiro[cyclopropane-1,2′-pyrrolizin]-7a′(5′H)-yl)methoxy)pyrido[4,3-d]pyrimidin-7-yl)naphthalen-2-ol isomer 1;
5-ethynyl-6-fluoro-4-(8-fluoro-4-(1,4-oxazepan-4-yl)-2-(((6′R,7a′R)-2,2,6′-trifluorodihydro-1′H,3′H-spiro[cyclopropane-1,2′-pyrrolizin]-7a′(5′H)-yl)methoxy)pyrido[4,3-d]pyrimidin-7-yl)naphthalen-2-ol isomer 2;
5-ethyl-6-fluoro-4-(8-fluoro-4-(6-(hydroxymethyl)-1,4-oxazepan-4-yl)-2-(((4aS,7aR)-1-methyloctahydro-4aH-cyclopenta[b]pyridin-4a-yl)methoxy)pyrido[4,3-d]pyrimidin-7-yl)naphthalen-2-ol;
5-ethynyl-6-fluoro-4-(8-fluoro-4-(3-methyl-1,4-oxazepan-4-yl)-2-(((4aS,7aR)-1-methyloctahydro-4aH-cyclopenta[b]pyridin-4a-yl)methoxy)pyrido[4,3-d]pyrimidin-7-yl)naphthalen-2-ol isomer 1;
1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-3-(fluoromethyl)piperidin-3-ol isomer 1;
1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-3-(fluoromethyl)piperidin-3-ol isomer 2;
4-(2-(((4aS,7aR)-1-ethyloctahydro-4aH-cyclopenta[b]pyridin-4a-yl)methoxy)-8-fluoro-4-(1,4-oxazepan-4-yl)pyrido[4,3-d]pyrimidin-7-yl)-5-ethynyl-6-fluoronaphthalen-2-ol;
5-ethyl-4-(2-(((4aS,7aR)-1-ethyloctahydro-4aH-cyclopenta[b]pyridin-4a-yl)methoxy)-8-fluoro-4-(1,4-oxazepan-4-yl)pyrido[4,3-d]pyrimidin-7-yl)-6-fluoronaphthalen-2-ol;
2-(1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)piperidin-3-yl)acetonitrile;
2-(1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-3-methylpiperidin-3-yl)acetonitrile;
5-ethynyl-6-fluoro-4-(8-fluoro-4-(3-(hydroxymethyl)azepan-1-yl)-2-(((4aS,7aR)-1-methyloctahydro-4aH-cyclopenta[b]pyridin-4a-yl)methoxy)pyrido[4,3-d]pyrimidin-7-yl)naphthalen-2-ol isomer 1;
4-(4-((S)-3-(2,2-difluoroethyl)piperidin-1-yl)-8-fluoro-2-(((4aS,7aR)-1-methyloctahydro-4aH-cyclopenta[b]pyridin-4a-yl)methoxy)pyrido[4,3-d]pyrimidin-7-yl)-5-ethynyl-6-fluoronaphthalen-2-ol;
6-(difluoromethyl)-4-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-1,4-oxazepan-6-ol;
4-(2-(((4aS,7aR)-1-cyclopropyloctahydro-4aH-cyclopenta[b]pyridin-4a-yl)methoxy)-8-fluoro-4-(1,4-oxazepan-4-yl)pyrido[4,3-d]pyrimidin-7-yl)-5-ethynyl-6-fluoronaphthalen-2-ol;
2-(4-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-1,4-oxazepan-6-yl)acetonitrile isomer 1;
2-(4-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-1,4-oxazepan-6-yl)acetonitrile isomer 2;
1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-3-(trifluoromethyl)piperidin-3-ol isomer 1;
1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-3-(trifluoromethyl)piperidin-3-ol isomer 2;
4-(7-(8-ethyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-6-methyl-1,4-oxazepan-6-ol;
5-ethynyl-6-fluoro-4-(8-fluoro-2-(((2R,7aS)-2-fluorotetrahydro-1H-pyrrolizin-7a(5H)-yl)methoxy)-4-(2-methyl-1,4-oxazepan-4-yl)pyrido[4,3-d]pyrimidin-7-yl)naphthalen-2-ol isomer 1;
4-(4-(2-oxa-5-azabicyclo[4.1.0]heptan-5-yl)-8-fluoro-2-(((4aS,7aR)-1-methyloctahydro-4aH-cyclopenta[b]pyridin-4a-yl)methoxy)pyrido[4,3-d]pyrimidin-7-yl)-5-ethynyl-6-fluoronaphthalen-2-ol;
5-ethyl-6-fluoro-4-(8-fluoro-2-(((4aS,7aR)-1-methyloctahydro-4aH-cyclopenta[b]pyridin-4a-yl)methoxy)-4-(1,4-oxazepan-4-yl)pyrido[4,3-d]pyrimidin-7-yl)naphthalen-2-ol;
5-ethynyl-6-fluoro-4-(8-fluoro-2-(((2R,7aS)-2-fluorotetrahydro-1H-pyrrolizin-7a(5H)-yl)methoxy)-4-(5-methyl-1,4-oxazepan-4-yl)pyrido[4,3-d]pyrimidin-7-yl)naphthalen-2-ol;
3-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-3-azabicyclo[3.2.1]octan-6-ol;
5-ethynyl-6-fluoro-4-(8-fluoro-4-(5-methyl-1,4-oxazepan-4-yl)-2-(((4aS,7aR)-1-methyloctahydro-4aH-cyclopenta[b]pyridin-4a-yl)methoxy)pyrido[4,3-d]pyrimidin-7-yl)naphthalen-2-ol;
5-chloro-4-(8-fluoro-2-(((4aS,7aR)-1-methyloctahydro-4aH-cyclopenta[b]pyridin-4a-yl)methoxy)-4-(1,4-oxazepan-4-yl)pyrido[4,3-d]pyrimidin-7-yl)naphthalen-2-ol;
5-ethynyl-6-fluoro-4-(8-fluoro-4-(5-methyl-1,4-oxazepan-4-yl)-2-(((4aS,7aR)-1-methyloctahydro-4aH-cyclopenta[b]pyridin-4a-yl)methoxy)pyrido[4,3-d]pyrimidin-7-yl)naphthalen-2-ol;
5-ethynyl-6-fluoro-4-(8-fluoro-2-(((2R,7aS)-2-fluorotetrahydro-1H-pyrrolizin-7a(5H)-yl)methoxy)-4-(2-methyl-1,4-oxazepan-4-yl)pyrido[4,3-d]pyrimidin-7-yl)naphthalen-2-ol isomer 2;
1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-3-methylazepan-3-ol;
4-(4-(3-azabicyclo[3.2.1]octan-3-yl)-8-fluoro-2-(((4aS,7aR)-1-methyloctahydro-4aH-cyclopenta[b]pyridin-4a-yl)methoxy)pyrido[4,3-d]pyrimidin-7-yl)-5-ethynyl-6-fluoronaphthalen-2-ol;
5-ethynyl-6-fluoro-4-(8-fluoro-4-(3-(hydroxymethyl)azepan-1-yl)-2-(((4aS,7aR)-1-methyloctahydro-4aH-cyclopenta[b]pyridin-4a-yl)methoxy)pyrido[4,3-d]pyrimidin-7-yl)naphthalen-2-ol isomer 2;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol;
4-(7-(8-Ethyl-7-fluoro-3-hydroxynaphthalen-1-yl)-8-fluoro-2-(((4aS,7aR)-1-methyloctahydro-4aH-cyclopenta[b]pyridin-4a-yl)methoxy)quinazolin-4-yl)-6-methyl-1,4-oxazepan-6-ol;
4-(2-{[(4aS,7aR)-1-methyl-octahydro-1H-cyclopenta[b]pyridin-4a-yl]methoxy}-8-fluoro-5-methoxy-4-(1,4-oxazepan-4-yl)pyrido[4,3-d]pyrimidin-7-yl)-5-ethyl-6-fluoronaphthalen-2-ol;
(3R)-1-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoro-5-methoxypyrido[4,3-d]pyrimidin-4-yl)-3-methylpiperidin-3-ol;
(3R)-1-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoro-5-methoxypyrido[4,3-d]pyrimidin-4-yl)-3-methylpiperidin-3-ol;
5-ethynyl-6-fluoro-4-(8-fluoro-5-methoxy-4-(1,4-oxazepan-4-yl)-2-(((6′R,7a′R)-2,2,6′-trifluorodihydro-1′H,3′H-spiro[cyclopropane-1,2′-pyrrolizin]-7a′(5′H)-yl)methoxy)pyrido[4,3-d]pyrimidin-7-yl)naphthalen-2-ol isomer 1;
5-ethynyl-6-fluoro-4-(8-fluoro-5-methoxy-4-(1,4-oxazepan-4-yl)-2-(((6′R,7a′R)-2,2,6′-trifluorodihydro-1′H,3′H-spiro[cyclopropane-1,2′-pyrrolizin]-7a′(5′H)-yl)methoxy)pyrido[4,3-d]pyrimidin-7-yl)naphthalen-2-ol isomer 2;
4-(4-((1R,4R)-2-oxa-5-azabicyclo[2.2.1]heptan-5-yl)-8-fluoro-2-(((2R,7aS)-2-fluorotetrahydro-1H-pyrrolizin-7a(5H)-yl)methoxy)-5-methoxypyrido[4,3-d]pyrimidin-7-yl)-5-ethynyl-6-fluoronaphthalen-2-ol;
(R)-1-(7-(8-ethyl-7-fluoro-3-hydroxynaphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-yl)-3-methylpiperidin-3-ol;
4-(4-((1R,4R)-2-oxa-5-azabicyclo[2.2.1]heptan-5-yl)-8-fluoro-2-(((2R,7aS)-2-fluorotetrahydro-1H-pyrrolizin-7a(5H)-yl)methoxy)-5-methoxypyrido[4,3-d]pyrimidin-7-yl)-5-ethyl-6-fluoronaphthalen-2-ol;
(R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)-5-methoxypyrido[4,3-d]pyrimidin-4-yl)-3-methylpiperidin-3-ol;
4-(4-((1R,4R)-2-oxa-5-azabicyclo[2.2.1]heptan-5-yl)-8-fluoro-5-methoxy-2-(((4aS,7aR)-1-methyloctahydro-4aH-cyclopenta[b]pyridin-4a-yl)methoxy)pyrido[4,3-d]pyrimidin-7-yl)-5-ethyl-6-fluoronaphthalen-2-ol;
4-(4-(6,6-difluoro-1,4-oxazepan-4-yl)-8-fluoro-2-(((2R,7aS)-2-fluorotetrahydro-1H-pyrrolizin-7a(5H)-yl)methoxy)-5-methoxypyrido[4,3-d]pyrimidin-7-yl)-5-ethynyl-6-fluoronaphthalen-2-ol;
4-(4-(6,6-difluoro-1,4-oxazepan-4-yl)-8-fluoro-5-methoxy-2-(((4aS,7aR)-1-methyloctahydro-4aH-cyclopenta[b]pyridin-4a-yl)methoxy)pyrido[4,3-d]pyrimidin-7-yl)-5-ethynyl-6-fluoronaphthalen-2-ol;
4-(4-(2-oxa-5-azabicyclo[4.1.0]heptan-5-yl)-8-fluoro-2-(((2R,7aS)-2-fluorotetrahydro-1H-pyrrolizin-7a(5H)-yl)methoxy)-5-methoxypyrido[4,3-d]pyrimidin-7-yl)-5-ethynyl-6-fluoronaphthalen-2-ol;
4-(4-(2-oxa-5-azabicyclo[4.1.0]heptan-5-yl)-8-fluoro-5-methoxy-2-(((4aS,7aR)-1-methyloctahydro-4aH-cyclopenta[b]pyridin-4a-yl)methoxy)pyrido[4,3-d]pyrimidin-7-yl)-5-ethynyl-6-fluoronaphthalen-2-ol;
4-(4-((1R,4R)-2-oxa-5-azabicyclo[2.2.1]heptan-5-yl)-8-fluoro-5-methoxy-2-(((4aS,7aR)-1-methyloctahydro-4aH-cyclopenta[b]pyridin-4a-yl)methoxy)pyrido[4,3-d]pyrimidin-7-yl)-5-ethynyl-6-fluoronaphthalen-2-ol;
5-ethynyl-6-fluoro-4-(8-fluoro-4-(6-fluoro-6-methyl-1,4-oxazepan-4-yl)-2-(((4aS,7aR)-1-methyloctahydro-4aH-cyclopenta[b]pyridin-4a-yl)methoxy)pyrido[4,3-d]pyrimidin-7-yl)naphthalen-2-ol;
3-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-3-azabicyclo[4.1.0]heptan-1-ol;
4-(8-fluoro-2-(((2R,7aS)-2-fluorotetrahydro-1H-pyrrolizin-7a(5H)-yl)methoxy)-7-(4-(trifluoromethyl)-1H-indol-3-yl)pyrido[4,3-d]pyrimidin-4-yl)-6-methyl-1,4-oxazepan-6-ol;
1-(8-fluoro-2-(((2R,7aS)-2-fluorotetrahydro-1H-pyrrolizin-7a(5H)-yl)methoxy)-7-(4-(trifluoromethyl)-1H-indol-3-yl)pyrido[4,3-d]pyrimidin-4-yl)-3-(trifluoromethyl)piperidin-3-ol;
1-(8-fluoro-2-(((2R,7aS)-2-fluorotetrahydro-1H-pyrrolizin-7a(5H)-yl)methoxy)-7-(4-(trifluoromethyl)-1H-indol-3-yl)pyrido[4,3-d]pyrimidin-4-yl)-3-(trifluoromethyl)piperidin-3-ol;
4-(8-fluoro-2-(((2R,7aS)-2-fluorotetrahydro-1H-pyrrolizin-7a(5H)-yl)methoxy)-7-(4-(trifluoromethyl)-1H-indol-3-yl)pyrido[4,3-d]pyrimidin-4-yl)-1,4-oxazepane;
3-(difluoromethyl)-1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)piperidin-3-ol;
3-(difluoromethyl)-1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)piperidin-3-ol;
5-ethynyl-6-fluoro-4-(8-fluoro-2-(((3S,4aS,7aR)-3-fluoro-1-methyloctahydro-4aH-cyclopenta[b]pyridin-4a-yl)methoxy)-4-(1,4-oxazepan-4-yl)pyrido[4,3-d]pyrimidin-7-yl)naphthalen-2-ol;
5-ethynyl-6-fluoro-4-(8-fluoro-2-(((4aS,7aR)-1-methyloctahydro-4aH-cyclopenta[b]pyridin-4a-yl)methoxy)-4-(1,4-oxazocan-4-yl)pyrido[4,3-d]pyrimidin-7-yl)naphthalen-2-ol;
(3R)-1-(7-(5-chloro-6-methyl-1H-indazol-4-yl)-8-fluoro-2-(((4aS)-1-methyloctahydro-4aH-cyclopenta[b]pyridin-4a-yl)methoxy)pyrido[4,3-d]pyrimidin-4-yl)-3-methylpiperidin-3-ol;
6-cyclopropyl-4-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)pyrido[4,3-d]pyrimidin-4-yl)-1,4-oxazepan-6-ol;
1-(7-(8-ethyl-7-fluoro-3-hydroxynaphthalen-1-yl)-8-fluoro-2-(((4aS,7aR)-1-methyloctahydro-4aH-cyclopenta[b]pyridin-4a-yl)methoxy)quinazolin-4-yl)-3-(trifluoromethyl)piperidin-3-ol;
(R)-1-(7-(8-ethyl-7-fluoro-3-hydroxynaphthalen-1-yl)-8-fluoro-2-(((4aS,7aR)-1-methyloctahydro-4aH-cyclopenta[b]pyridin-4a-yl)methoxy)quinazolin-4-yl)-3-methylpiperidin-3-ol;
(3R)-1-(7-(8-ethynyl-7-fluoro-3-hydroxynaphthalen-1-yl)-8-fluoro-2-((1-methyloctahydro-3aH-indol-3a-yl)methoxy)quinazolin-4-yl)-3-methylpiperidin-3-ol;
1-(7-(8-ethyl-7-fluoro-3-hydroxynaphthalen-1-yl)-8-fluoro-2-(((4aS,7aR)-1-methyloctahydro-4aH-cyclopenta[b]pyridin-4a-yl)methoxy)quinazolin-4-yl)-3-(trifluoromethyl)piperidin-3-ol;
1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)quinazolin-4-yl)-3-(trifluoromethyl)piperidin-3-ol;
1-(7-(8-ethynyl-7-fluoro-3-hydroxynaphthalen-1-yl)-8-fluoro-2-(((4aS,7aR)-1-methyloctahydro-4aH-cyclopenta[b]pyridin-4a-yl)methoxy)quinazolin-4-yl)-3-(trifluoromethyl)piperidin-3-ol;
(3R)-1-(7-(8-ethynyl-7-fluoro-3-hydroxynaphthalen-1-yl)-8-fluoro-2-((1-methyloctahydro-3aH-indol-3a-yl)methoxy)quinazolin-4-yl)-3-methylpiperidin-3-ol;
3-(difluoromethyl)-1-(7-(8-ethynyl-7-fluoro-3-hydroxynaphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-yl)piperidin-3-ol;
3-(difluoromethyl)-1-(7-(8-ethynyl-7-fluoro-3-hydroxynaphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-yl)piperidin-3-ol;
(4aS,7aR)-4a-(((7-(8-ethynyl-7-fluoro-3-hydroxynaphthalen-1-yl)-8-fluoro-4-((R)-3-hydroxy-3-methylpiperidin-1-yl)-5-methoxypyrido[4,3-d]pyrimidin-2-yl)oxy)methyl)-1-methyloctahydro-1H-cyclopenta[b]pyridine 1-oxide;
1-(7-(8-ethynyl-7-fluoro-3-hydroxynaphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-yl)-3-(trifluoromethyl)piperidin-3-ol;
5-ethynyl-6-fluoro-4-(8-fluoro-5-methyl-2-(((4aS,7aR)-1-methyloctahydro-4aH-cyclopenta[b]pyridin-4a-yl)methoxy)-4-(1,4-oxazepan-4-yl)pyrido[4,3-d]pyrimidin-7-yl)naphthalen-2-ol;
1-(7-(8-ethynyl-7-fluoro-3-hydroxynaphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-yl)-3-(trifluoromethyl)piperidin-3-ol;
4-(2-(((4aS,7aR)-1-cyclopropyloctahydro-4aH-cyclopenta[b]pyridin-4a-yl)methoxy)-8-fluoro-5-methoxy-4-(1,4-oxazepan-4-yl)pyrido[4,3-d]pyrimidin-7-yl)-5-ethynyl-6-fluoronaphthalen-2-ol;
4-(5-(difluoromethoxy)-8-fluoro-2-(((4aS,7aR)-1-methyloctahydro-4aH-cyclopenta[b]pyridin-4a-yl)methoxy)-4-(1,4-oxazepan-4-yl)pyrido[4,3-d]pyrimidin-7-yl)-5-ethynyl-6-fluoronaphthalen-2-ol;
(R)-1-(7-(8-ethynyl-7-fluoro-3-hydroxynaphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-yl)-3-(fluoromethyl)piperidin-3-ol;
(R)-1-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)-5-methoxypyrido[4,3-d]pyrimidin-4-yl)-3-(fluoromethyl)piperidin-3-ol;
5-ethynyl-6-fluoro-4-(8-fluoro-2-(((4aS,7aR)-3-fluoro-1-(methyl-d3)octahydro-4aH-cyclopenta[b]pyridin-4a-yl)methoxy)-5-methoxy-4-(1,4-oxazepan-4-yl)pyrido[4,3-d]pyrimidin-7-yl)naphthalen-2-ol;
5-ethynyl-6-fluoro-4-(8-fluoro-2-(((4aS,7aR)-3-fluoro-1-(methyl-d3)octahydro-4aH-cyclopenta[b]pyridin-4a-yl)methoxy)-5-methoxy-4-(1,4-oxazepan-4-yl)pyrido[4,3-d]pyrimidin-7-yl)naphthalen-2-ol;
4-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)-5-methoxypyrido[4,3-d]pyrimidin-4-yl)-6-methyl-1,4-oxazepan-6-ol;
5-ethynyl-6-fluoro-4-(8-fluoro-5-methoxy-2-(((4aS,7aR)-1-methyloctahydro-4aH-cyclopenta[b]pyridin-4a-yl)methoxy)-4-(1,4-oxazocan-4-yl)pyrido[4,3-d]pyrimidin-7-yl)naphthalen-2-ol;
5-ethynyl-6-fluoro-4-(8-fluoro-2-(((3R,4aS,7aR)-3-fluoro-1-methyloctahydro-4aH-cyclopenta[b]pyridin-4a-yl)methoxy)-5-methoxy-4-(1,4-oxazepan-4-yl)pyrido[4,3-d]pyrimidin-7-yl)naphthalen-2-ol;
5-ethynyl-6-fluoro-4-(8-fluoro-2-(((3S,4aS,7aR)-3-fluoro-1-methyloctahydro-4aH-cyclopenta[b]pyridin-4a-yl)methoxy)-5-methoxy-4-(1,4-oxazepan-4-yl)pyrido[4,3-d]pyrimidin-7-yl)naphthalen-2-ol;
1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)-5-methoxypyrido[4,3-d]pyrimidin-4-yl)-3-(trifluoromethyl)piperidin-3-ol;
1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)-5-methoxypyrido[4,3-d]pyrimidin-4-yl)-3-(trifluoromethyl)piperidin-3-ol;
1-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)-5-methoxypyrido[4,3-d]pyrimidin-4-yl)-3-(trifluoromethyl)piperidin-3-ol;
1-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)-5-methoxypyrido[4,3-d]pyrimidin-4-yl)-3-(trifluoromethyl)piperidin-3-ol;
5-ethynyl-6-fluoro-4-(8-fluoro-5-methoxy-2-(((4aS,7aR)-1-(methyl-d3)octahydro-4aH-cyclopenta[b]pyridin-4a-yl)methoxy)-4-(1,4-oxazepan-4-yl)pyrido[4,3-d]pyrimidin-7-yl)naphthalen-2-ol;
(R)-1-(2-((1′H,3′H,5′H-dispiro[cyclopropane-1,2′-pyrrolizine-6′,1″-cyclopropan]-7a′(7′H)-yl)methoxy)-7-(8-ethynyl-7-fluoro-3-hydroxynaphthalen-1-yl)-8-fluoro-5-methoxypyrido[4,3-d]pyrimidin-4-yl)-3-methylpiperidin-3-ol;
4-(4-(2-oxa-5-azabicyclo[4.1.0]heptan-5-yl)-8-fluoro-2-(((2R,7aS)-2-fluorotetrahydro-1H-pyrrolizin-7a(5H)-yl)methoxy)-5-methoxypyrido[4,3-d]pyrimidin-7-yl)-5-ethynyl-6-fluoronaphthalen-2-ol;
4-(4-(2-oxa-5-azabicyclo[4.1.0]heptan-5-yl)-8-fluoro-5-methoxy-2-(((4aS,7aR)-1-methyloctahydro-4aH-cyclopenta[b]pyridin-4a-yl)methoxy)pyrido[4,3-d]pyrimidin-7-yl)-5-ethynyl-6-fluoronaphthalen-2-ol;
(3R)-1-(7-(5-chloro-6-methyl-1H-indazol-4-yl)-8-fluoro-2-(((2R,7aS)-2-fluorotetrahydro-1H-pyrrolizin-7a(5H)-yl)methoxy)-5-methoxypyrido[4,3-d]pyrimidin-4-yl)-3-methylpiperidin-3-ol, and
4-(2-{[(4aS,7aR)-1-methyl-octahydro-1H-cyclopenta[b]pyridin-4a-yl]methoxy}-7-(8-ethyl-7-fluoro-3-hydroxynaphthalen-1-yl)-8-fluoropyrido[4,3-d]pyrimidin-4-yl)-6-methyl-1,4-oxazepan-6-ol;
or a pharmaceutically acceptable salt thereof.

In some aspects, the present disclosure provides an atropisomer of a compound of any of the prior aspects. In certain embodiments, the compound is a stable atropisomer as described herein.

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of Formula (I) or a pharmaceutically acceptable salt thereof” includes, but is not limited to, a compound of Formula (I), two compounds of Formula (I), a pharmaceutically acceptable salt of a compound of Formula (I), a compound of Formula (I) and one or more pharmaceutically acceptable salts of the compound of Formula (I), and two or more pharmaceutically acceptable salts of a compound of Formula (I).

The term “C₂-C₄alkenyl,” as used herein, refers to a group derived from a hydrocarbon containing two to four carbon atoms and one double bond.

The term “C₁-C₃alkoxy,” as used herein, refers to a C₁-C₃alkyl group attached to the parent molecular moiety through an oxygen atom.

The term “C₁-C₆alkoxy,” as used herein, refers to a C₁-C₆alkyl group attached to the parent molecular moiety through an oxygen atom.

The term “C₁-C₃alkoxyC₁-C₃alkyl,” as used herein, refers to a C₁-C₃alkoxy group attached to the parent molecular moiety through a C₁-C₃alkyl group.

The term “C₁-C₆alkoxyC₁-C₆alkyl,” as used herein, refers to a C₁-C₆alkoxy group attached to the parent molecular moiety through a C₁-C₆alkyl group.

The term “C₁-C₃alkoxycarbonyl,” as used herein, refers to a C₁-C₃alkoxy group attached to the parent molecular moiety through a carbonyl group.

The term “C₁-C₃alkyl,” as used herein, refers to a group derived from a straight or branched chain saturated hydrocarbon containing from one to three carbon atoms.

The term “C₁-C₆alkyl,” as used herein, refers to a group derived from a straight or branched chain saturated hydrocarbon containing from one to six carbon atoms.

The term “(C₁-C₆alkyl)amino,” as used herein, refers to R—NH, wherein R is a C₁-C₆alkyl group.

The term “(C₁-C₆alkyl)aminoC₁-C₃alkyl,” as used herein, refers to a(C₁-C₆alkyl)amino group attached to the parent molecular moiety through a C₁-C₃alkyl group.

The term “C₁-C₃alkylcarbonyl,” as used herein, refers to a C₁-C₃alkyl group attached to the parent molecular moiety through a carbonyl group.

The term “C₁-C₃alkylene,” as used herein, refers to a divalent straight chain saturated hydrocarbon containing from one to three carbon atoms,

The term “haloC₁-C₃alkylcarbonyl,” as used herein, refers to a haloC₁-C₃alkyl group attached to the parent molecular moiety through a carbonyl group. In some aspects, the haloC₁-C₃alkylcarbonyl is —C(O)CF₃.

The term “C₂-C₄alkynyl,” as used herein, refers to a group derived from a hydrocarbon containing two to four carbon atoms and one triple bond.

The term “C₂-C₄alkynyloxy,” as used herein, refers to a C₂-C₄alkynyl group attached to the parent molecular moiety through an oxygen atom.

The term “amino,” as used herein, refers to —NH₂.

The term “aminoC₁-C₃alkyl,” as used herein, refers to an amino group attached to the parent molecular moiety through a C₁-C₃alkyl group.

The term “aryl,” as used herein, refers to a phenyl group, or a bicyclic or tricyclic ring system wherein at least one of the rings is a phenyl group. Bicyclic and tricyclic fused ring systems consist of a phenyl group fused to a four- to eight-membered aromatic or non-aromatic monocyclic or bicyclic fused or spirocyclic ring system. The aryl groups of the present disclosure can be attached to the parent molecular moiety through any substitutable carbon atom in the group. Representative examples of aryl groups include, but are not limited to, indanyl, indenyl, naphthyl, phenyl, tetrahydronaphthyl, and 2′,3′-dihydrospiro(cyclopropane-1,1′-indenyl).

The term “arylC₁-C₆alkyl,” as used herein refers to an aryl group attached to the parent molecular moiety through a C₁-C₆alkyl group.

The term “carboxy,” as used herein, refers to —CO₂H.

The term “carboxyC₁-C₆alkyl,” as used herein, refers to a C₁-C₆alkyl group substituted with one, two, or three carboxy groups.

The term “cyano,” as used herein, refers to —CN.

The term “cyanoC₁-C₃alkoxy,” as used herein, refers to a C₁-C₃alkoxy group substituted by a cyano group.

The term “C₃-C₄cycloalkyl,” as used herein, refers to a saturated monocyclic hydrocarbon ring system having three or four carbon atoms and zero heteroatoms (e.g., cyclopropyl, cyclobutyl).

The term “C₃-C₆cycloalkyl,” as used herein, refers to a saturated monocyclic or bicyclic hydrocarbon ring system having three to six carbon atoms and zero heteroatoms (e.g., cyclopropyl, cyclobutyl, cyclopentyl, bicyclo[1.1.1]pentanyl, cyclohexyl).

The term “C₃-C₈cycloalkyl,” as used herein, refers to a saturated monocyclic or bicyclic hydrocarbon ring system having three, four, five, six, seven, or eight carbon atoms and zero heteroatoms (e.g., cyclopropyl, cyclobutyl, cyclopentyl, bicyclo[1.1.1]pentanyl). In some embodiments, the C₃-C₈cycloalkyl is fused to a heterocyclyl or heteroaryl, as described herein.

The term “C₃-C₆cycloalkylC₁-C₆alkyl,” as used herein, refers to a C₃-C₆cycloalkyl (e.g., cyclopropyl, cyclobutyl, cyclopentyl, cyclohexyl) attached to the parent molecular moiety through a C₁-C₆alkyl group.

The term “C₃-C₆cycloalkylcarbonyl,” as used herein, refers to a C₃-C₆cycloalkyl group (e.g., cyclopropyl, cyclobutyl, cyclopentyl, cyclohexyl) attached to the parent molecular moiety through a carbonyl group.

The term “di(C₁-C₆alkyl)amino,” as used herein, refers to —NR^{z}R^{z′}, wherein R^{z} and R^{z′} are the same or different C₁-C₆alkyl groups.

The term “di(C₁-C₃alkyl)aminoC₂-C₆alkyl,” as used herein, refers to —(C₂-C₆alkyl)NR^{z}R^{z′}, wherein R^{z} and R^{z′} are the same or different C₁-C₆alkyl groups.

The term “dimethylphosphino,” as used herein, refers to —P(O)(CH₃)₂.

The term “dimethylsulfonamide,” as used herein, refers to —S(O)₂N(CH₃)₂.

The terms “halo” and “halogen,” as used herein, refer to F, Cl, Br, or I.

The term “haloC₁-C₃alkoxy,” as used herein, refers to a C₁-C₃alkoxy group substituted with one, two, or three halogen atoms.

The term “haloC₁-C₃alkyl,” as used herein, refers to a C₁-C₃alkyl group substituted with one, two, or three halogen atoms.

The term “haloC₁-C₆alkyl,” as used herein, refers to a C₁-C₆alkyl group substituted with one, two, or three halogen atoms.

The term “heteroaryl,” as used herein, refers to an aromatic five- or six-membered ring where at least one atom is selected from N, O, and S, and the remaining atoms are carbon. The term “heteroaryl” also includes bicyclic systems where a heteroaryl ring is fused to a four- to six-membered aromatic or non-aromatic ring containing zero, one, or two additional heteroatoms selected from N, O, and S; and tricyclic systems where a bicyclic system is fused to a four- to six-membered aromatic or non-aromatic ring containing zero, one, or two additional heteroatoms selected from N, O, and S. The heteroaryl groups are attached to the parent molecular moiety through any substitutable carbon or nitrogen atom in the group. Representative examples of heteroaryl groups include, but are not limited to, alloxazine, benzo[1,2-d:4,5-d′]bisthiazole, benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, purine, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl.

The term “heteroarylC₁-C₆alkyl, as used herein, refers to a heteroaryl group attached to the parent molecular moiety through a C₁-C₆alkyl group.

The term “heterocyclyl,” as used herein, refers to a four-, five-, six-, seven-, eight-, nine-, ten-, eleven-, or twelve-membered saturated or partially unsaturated ring containing one, two, or three heteroatoms independently selected from nitrogen, oxygen, and sulfur. The term “heterocyclyl” also includes groups in which the heterocyclyl ring is fused to one, two, or three four- to six-membered aromatic or non-aromatic carbocyclic rings or monocyclic heterocyclyl groups. The term “heterocyclyl” also includes monocyclic or polycyclic heterocyclyl group as described above which are further substituted with one or more spirocyclic groups that are attached to the heterocyclyl group through a spiro carbon. Examples of heterocyclyl groups include, but are not limited to, dihydro-1′H,3′H,5′H-dispiro[cyclopropane-1,2′-pyrrolizine-6′,1″-cyclopropane], hexahydro-2H-1,4-dioxa-2a1-azacyclopenta[cd]pentalenyl, hexahydropyrrolizinyl, indolinyl, morpholinyl, octahydroindolizinyl, octahydroquinolizinyl, piperazinyl, piperidinyl, pyrrolidinyl, tetrahydrofuranyl, tetrahydropyranyl, oxetanyl, azetidinyl, thiatanyl, thiolanyl, thienyl, 1,2-dioxanyl, 1,4-dioxanyl, triemethylenyl oxide, thiazolinyl, imidazolidinyl, homopiperazinyl, pyrrolinyl, tetrahydrothiofuranyl, pyranyl, and thiomorpholinyl. The term “4- to 6-membered heterocyclyl,” refers to those “heterocyclyl” groups described above that include four, five, or six members in the ring.

In particular, when R⁵ is a five- to ten-membered monocyclic, bicyclic, or tricyclic ring containing one nitrogen atom and optionally containing one to three additional heteroatoms selected from the group consisting of oxygen or nitrogen, wherein the ring contains zero to three double bonds the bicyclic or tricyclic ring can be formed by fusion of an additional ring or the additional ring can be a spiro ring. Similarly, when R^{c} and R^{d}, together with the nitrogen atom to which they are attached, form a five- to ten-membered ring monocyclic, bicyclic or tricyclic ring optionally containing one to three additional heteroatoms selected from the group consisting of nitrogen, oxygen, and sulfur, the bicyclic or tricyclic ring can be formed by fusion of a second ring or the second ring can be a spiro ring, similar to as described in the preceding paragraph.

The term “heterocyclylC₁-C₃alkyl,” as used herein, refers to a heterocyclyl group attached to the parent molecular moiety through a C₁-C₃alkyl group.

The term “heterocyclylC₁-C₆alkyl,” as used herein, refers to a heterocyclyl group attached to the parent molecular moiety through a C₁-C₆alkyl group.

The term “hydroxy,” as used herein, refers to —OH.

The term “hydroxyC₁-C₃alkyl,” as used herein, refers to a hydroxy group attached to the parent molecular moiety through a C₁-C₃alkyl group.

The term “hydroxyC₁-C₆alkyl,” as used herein, refers to a hydroxy group attached to the parent molecular moiety through a C₁-C₆alkyl group.

The term “methylsulfonyl,” as used herein, refers to —S(O)₂CH₃.

The term “oxo,” as used herein, refers to =0.

The term “tetrahydropyranylcarbonyl,” as used herein, refers a tetrahydropyranyl group attached to the parent molecular moiety through a carbonyl group. The carbonyl can be attached to the tetrahydropyranyl moiety at any suitable position, such as the 1-, 2-, 3-, or 4-position. In an aspect, the carbonyl is attached to the tetrahydropyranyl group at the 4-position.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a compound described herein can be prepared using a radioactive isotope and the resulting radiolabeled compound can be used to develop a binding assay or for metabolism studies. Alternatively, and for the same purpose, a compound described herein can be converted to a radiolabeled form by catalytic tritiation using methods known to those skilled in the art.

Certain compounds of the present disclosure exist as stereoisomers. It should be understood that when stereochemistry is not specified, the present disclosure encompasses all stereochemical isomeric forms, or mixtures thereof, which possess the ability inhibit mutant KRAS. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure exist as atropisomers. The term “atropisomers” refers to conformational stereoisomers which occur when rotation about a single bond in the molecule is prevented, or greatly slowed, as a result of steric interactions with other parts of the molecule and the substituents at both ends of the single bond are asymmetrical (i.e., optical activity arises without requiring an asymmetric carbon center or stereocenter). Where the rotational barrier about the single bond is high enough, and interconversion between conformations is slow enough, separation and isolation of the isomeric species may be permitted. Atropisomers are enantiomers (or epimers) without a single asymmetric atom.

The atropisomers can be considered stable if the barrier to interconversion is high enough to permit the atropisomers to undergo little or no interconversion at room temperature for at least a week. In some aspects the atropisomers undergo little or no interconversion at room temperature for at least a year. In some aspects, an atropisomeric compound of the disclosure does not undergo more than about 5% interconversion to its opposite atropisomer at room temperature during one week when the atropisomeric compound is in substantially pure form, which is generally a solid state. In some aspects, an atropisomeric compound of the disclosure does not undergo more than about 5% interconversion to its opposite atropisomer at room temperature (approximately 25° C.) during one year. In some aspects, the atropisomeric compounds of the disclosure are stable enough to undergo no more than about 5% interconversion in an aqueous pharmaceutical formulation held at 0° C. for at least one week. The present chemical entities, pharmaceutical compositions and methods are meant to include all such possible atropisomers, including racemic mixtures, diastereomeric mixtures, epimeric mixtures, optically pure forms of single atropisomers, and intermediate mixtures.

The energy barrier to thermal racemization of atropisomers may be determined by the steric hindrance to free rotation of one or more bonds forming a chiral axis. Certain biaryl compounds exhibit atropisomerism where rotation around an interannular bond lacking C2 symmetry is restricted. The free energy barrier for isomerization (enantiomerization) is a measure of the stability of the interannular bond with respect to rotation. Optical and thermal excitation can promote racemization of such isomers, dependent on electronic and steric factors.

Ortho-substituted biaryl compounds may exhibit this type of conformational, rotational isomerism. Such biaryls are enantiomeric, chiral atropisomers where the sp²-sp² carbon-carbon, interannular bond between the aryl rings has a sufficiently high energy barrier to prevent free rotation, and where substituents W¹≠W² and W³≠W⁴ render the molecule asymmetric.

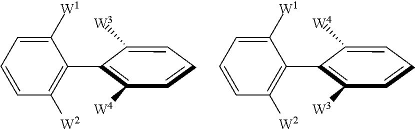

The steric interaction between W¹:W³, W¹:W⁴, and/or W²:W⁴, W²:W³ is large enough to make the planar conformation an energy maximum. Two non-planar, axially chiral enantiomers then exist as atropisomers when their interconversion is slow enough such that they can be isolated free of each other. Bold lines and dashed lines in the figures shown above indicate those moieties, or portions of the molecule, which are sterically restricted due to a rotational energy barrier. Balded moieties exist orthogonally above the plane of the page, and dashed moieties exist orthogonally below the plane of the page. The ‘flat’ part of the molecule (the left ring in each of the two depicted biaryls) is in the plane of the page.

The pharmaceutical compositions of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately be reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

### Pharmaceutical Compositions

In another aspect, the present disclosure provides a composition, e.g., a pharmaceutical composition, containing one or a combination of the compounds described within the present disclosure, formulated together with a pharmaceutically acceptable carrier. Pharmaceutical compositions of the disclosure also can be administered in combination therapy, i.e., combined with other agents, as described herein.

As used herein, “pharmaceutically acceptable carrier” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound can be coated in a material to protect the compound from the action of acids and other natural conditions that can inactivate the compound.

The pharmaceutical compositions of the present disclosure can be administered via one or more routes of administration using one or more of a variety of methods known in the art. As will be appreciated by the skilled artisan, the route and/or mode of administration will vary depending upon the desired results. In some aspects, the routes of administration for compounds of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are reduced pressure drying and freeze-drying (lyophilization) that yield a powder of the active ingredient plus any additional desired ingredient from a previously sterile-filtered solution thereof.

Examples of suitable aqueous and non-aqueous carriers that can be employed in the pharmaceutical compositions of the disclosure include water, ethanol, polyols (such as glycerol, propylene glycol, polyethylene glycol, and the like), and suitable mixtures thereof, vegetable oils, and injectable organic esters. Proper fluidity can be maintained, for example, by the use of coating materials, such as lecithin, by the maintenance of the required particle size in the case of dispersions, and by the use of surfactants.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution or as a liquid with ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, the compounds of the disclosure can be administered via a non-parenteral route, such as a topical, epidermal or mucosal route of administration, for example, intranasally, orally, vaginally, rectally, sublingually or topically.

Any pharmaceutical composition contemplated herein can, for example, be delivered orally via any acceptable and suitable oral preparation. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from the group consisting of sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one non-toxic pharmaceutically acceptable excipient suitable for the manufacture of tablets.

An aqueous suspension can be prepared, for example, by admixing at least one compound of Formula (I) and/or at least one pharmaceutically acceptable salt thereof with at least one excipient suitable for the manufacture of an aqueous suspension, including, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example, heptadecathylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil, sesame oil, and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described herein above, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent, at least one suspending agent, and/or at least one preservative. Suitable dispersing agents, wetting agents, and suspending agents are already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents, and coloring agents.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Robinson, J. R., ed., Sustained and Controlled Release Drug Delivery Systems, Marcel Dekker, Inc., New York (1978).

Therapeutic compositions can be administered with medical devices known in the art. For example, in one aspect, a therapeutic composition of the disclosure can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medication through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain aspects, the compounds of the present disclosure can be administered parenterally, i.e., by injection, including, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and/or infusion.

In some aspects, the compounds of the present disclosure can be administered orally, i.e, via a gelatin capsule, tablet, hard or soft capsule, or a liquid capsule.

### Use of KRAS Inhibitors/Methods of Treating

Administration of a therapeutic agent described herein may include administration of a therapeutically effective amount of therapeutic agent. The term “therapeutically effective amount” as used herein refers, without limitation, to an amount of a therapeutic agent to treat a condition treatable by administration of a composition comprising the KRAS inhibitors described herein. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration.

For administration of the compounds described herein, the dosage ranges from about 0.0001 to 100 mg/kg, and more usually 0.01 to 40 mg/kg, of the host body weight. An exemplary treatment regime entails administration once per day, bi-weekly, tri-weekly, weekly, once every two weeks, once every three weeks, once every four weeks, once a month, once every 3 months or once every three to 6 months.

The disclosed compounds strongly inhibit anchorage-independent cell growth and therefore have the potential to inhibit tumor metastasis. Accordingly, in another aspect the disclosure provides a method for inhibiting tumor metastasis, the method comprising administering an effective amount a pharmaceutical composition of comprising any of the compounds disclosed herein and a pharmaceutically acceptable carrier to a subject in need thereof.

Ras mutations including but not limited to KRAS mutations have also been identified in hematological malignancies (e.g., cancers that affect blood, bone marrow and/or lymph nodes). Accordingly, certain aspects are directed to administration of a disclosed compounds (e.g., in the form of a pharmaceutical composition) to a patient in need of treatment of a hematological malignancy. Such malignancies include, but are not limited to, leukemias and lymphomas. For example, the presently disclosed compounds can be used for treatment of diseases such as Acute lymphoblastic leukemia (ALL), Acute myelogenous leukemia (AML), Chronic lymphocytic leukemia (CLL), small lymphocytic lymphoma (SLL), Chronic myelogenous leukemia (CML), Acute monocytic leukemia (AMoL) and/or other leukemias. In other aspects, the compounds are useful for treatment of lymphomas such as all subtypes of Hodgkins lymphoma or non-Hodgkins lymphoma.

Determining whether a tumor or cancer comprises a KRAS mutation can be undertaken by assessing the nucleotide sequence encoding the KRAS protein, by assessing the amino acid sequence of KRAS protein, or by assessing the characteristics of a putative KRAS mutant protein. The sequence of wild-type human KRAS proteins is known in the art.

Methods for detecting a KRAS mutation are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some aspects, samples are evaluated for KRAS mutations including by real-time PCR. In real-time PCR, fluorescent probes specific for the KRAS mutation are used. When a mutation is present, the probe binds and fluorescence is detected. In some aspects, the KRAS mutation is identified using a direct sequencing method of specific regions (e.g., exon 2 and/or exon 3) in the KRAS gene, for example. This technique will identify all possible mutations in the region sequenced.

Methods for detecting a mutation in a KRAS protein are known by those of skill in the art. These methods include, but are not limited to, detection of a KRAS mutant using a binding agent (e.g., an antibody) specific for the mutant protein, protein electrophoresis and Western blotting, and direct peptide sequencing.

Methods for determining whether a tumor or cancer comprises a KRAS mutation can use a variety of samples. In some aspects, the sample is taken from a subject having a tumor or cancer. In some aspects, the sample is taken from a subject having a cancer or tumor. In some aspects, the sample is a fresh tumor/cancer sample. In some aspects, the sample is a frozen tumor/cancer sample. In some aspects, the sample is a formalin-fixed paraffin-embedded sample. In some aspects, the sample is processed to a cell lysate. In some aspects, the sample is processed to DNA or RNA. The disclosure also relates to a method of treating a hyperproliferative disorder in a mammal that comprises administering to said mammal a therapeutically effective amount of a compound of the present disclosure, or a pharmaceutically acceptable salt, ester, prodrug, solvate, hydrate or derivative thereof. In some aspects, said method relates to the treatment of cancer such as acute myeloid leukemia, cancer in adolescents, adrenocortical carcinoma childhood, AIDS-related cancers (e.g. Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, isletcell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some aspects, said method relates to the treatment of a non-cancerous hyperproliferative disorder such as benign hyperplasia of the skin (e.g., psoriasis), restenosis, or prostate (e.g., benign prostatic hypertrophy (BPH)).

In certain aspects, the disclosure relates to methods for treatment of lung cancers, the methods comprise administering an effective amount of any of the above-described compound (or a pharmaceutical composition comprising the same) to a subject in need thereof. In certain aspects the lung cancer is a non-small cell lung carcinoma (NSCLC), for example adenocarcinoma, squamous-cell lung carcinoma or large-cell lung carcinoma. In other aspects, the lung cancer is a small cell lung carcinoma. Other lung cancers treatable with the disclosed compounds include, but are not limited to, glandular tumors, carcinoid tumors and undifferentiated carcinomas. Subjects that can be treated with compounds of the disclosure, or pharmaceutically acceptable salt, ester, prodrug, solvate, tautomer, hydrate or derivative of said compounds, according to the methods of this disclosure include, for example, subjects that have been diagnosed as having acute myeloid leukemia, acute myeloid leukemia, cancer in adolescents, adrenocortical carcinoma childhood, AIDS-related cancers (e.g. Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, hodgkin lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-hodgkin lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasalcavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some aspects subjects that are treated with the compounds of the disclosure include subjects that have been diagnosed as having a non-cancerous hyperproliferative disorder such as benign hyperplasia of the skin (e. g., psoriasis), restenosis, or prostate (e. g., benign pro static hypertrophy (BPH)). The disclosure further provides methods of modulating a mutant KRAS protein activity by contacting the protein with an effective amount of a compound of the disclosure. Modulation can be inhibiting or activating protein activity. In some aspects, the disclosure provides methods of inhibiting protein activity by contacting the mutant KRAS protein with an effective amount of a compound of the disclosure in solution. In some aspects, the disclosure provides methods of inhibiting the mutant KRAS protein activity by contacting a cell, tissue, organ that express the protein of interest. In some aspects, the disclosure provides methods of inhibiting protein activity in a subject including but not limited to rodents and mammal (e.g., human) by administering into the subject an effective amount of a compound of the disclosure. In some aspects, the percentage modulation exceeds 25%, 30%, 40%, 50%, 60%, 70%, 80%, or 90%. In some aspects, the percentage of inhibiting exceeds 25%, 30%, 40%, 50%, 60%, 70%, 80%, or 90%.In some aspects, the disclosure provides methods of inhibiting KRAS activity in a cell by contacting said cell with an amount of a compound of the disclosure sufficient to inhibit the activity of a KRAS mutant in said cell. In some aspects, the disclosure provides methods of inhibiting mutant KRAS in a tissue by contacting said tissue with an amount of a compound of the disclosure sufficient to inhibit the activity of mutant KRAS in said tissue. In some aspects, the disclosure provides methods of inhibiting KRAS in an organism by contacting said organism with an amount of a compound of the disclosure sufficient to inhibit the activity of KRAS in said organism. In some aspects, the disclosure provides methods of inhibiting KRAS activity in an animal by contacting said animal with an amount of a compound of the disclosure sufficient to inhibit the activity of KRAS in said animal. In some aspects, the disclosure provides methods of inhibiting KRAS including in a mammal by contacting said mammal with an amount of a compound of the disclosure sufficient to inhibit the activity of KRAS in said mammal. In some aspects, the disclosure provides methods of inhibiting KRAS activity in a human by contacting said human with an amount of a compound of the disclosure sufficient to inhibit the activity of KRAS in said human. The present disclosure provides methods of treating a disease mediated by KRAS activity in a subject in need of such treatment. The present disclosure also provides methods for combination therapies in which an agent known to modulate other pathways, or other components of the same pathway, or even overlapping sets of target enzymes are used in combination with a compound of the present disclosure, or a pharmaceutically acceptable salt, ester, prodrug, solvate, tautomer, hydrate or derivative thereof. In one aspect, such therapy includes but is not limited to the combination of one or more compounds of the disclosure with chemotherapeutic agents, therapeutic antibodies, and radiation treatment.

Many chemotherapeutics are presently known in the art and can be used in combination with the compounds of the disclosure. In some aspects, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. In some aspects, the chemotherapeutic agent is an immunooncology (IO) agent that can enhance, stimulate, or upregulate the immune system.

The compounds described herein can be used in combination with the agents disclosed herein or other suitable agents, depending on the condition being treated. Hence, in some aspects the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some aspects of the separate administration protocol, a compound of the disclosure and any of the agents described above are administered a few minutes apart, or a few hours apart, or a few days apart.

The compounds can be made by methods known in the art including those described below and including variations within the skill of the art. Some reagents and intermediates are known in the art. Other reagents and intermediates can be made by methods known in the art using readily available materials. Any variables (e.g., numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

### Synthesis

Abbreviations used herein include: AA for ammonium acetate; ACN or MeCN for acetonitrile; BOC or Boc for tert-butoxycarbonyl; BOP for (benzotriazol-1-yloxytris(dimethylamino)phosphonium hexafluorophosphate); t-Bu or tBu for tert-butyl; DAST for diethylaminosulfur trifluoride; DCM for dichloromethane; DEA for diethanolamine; DIEA or DIPEA for diisopropylethylamine; DMA for dimethylacetamide; DMAP for N,N-dimethylaminopyridine; DMF for dimethylformamide; DMSO for dimethylsulfoxide; dppf for 1,1′-bis(diphenylphosphino)ferrocene; EtOAc for ethyl acetate; EtOH for ethanol; h for hours; IPA for isopropanol; LAH for lithium aluminum hydride; LCMS for liquid chromatography-mass spectrometry; MeOH for methanol; hexamethyldisilazide; min for minutes; MOM for methoxymethyl; Piv for pivaloyl; SNAr for nucleophilic aromatic substitution; TBAF for tetrabutylammonium fluoride; TEA for triethylamine; TFA for trifluoroacetic acid; THE for tetrahydrofuran; and RT for room temperature or retention time (context will dictate).

### General Schemes

The compounds described herein can be prepared according to the methodology outlined in Schemes 1-4 and in the Examples shown below.

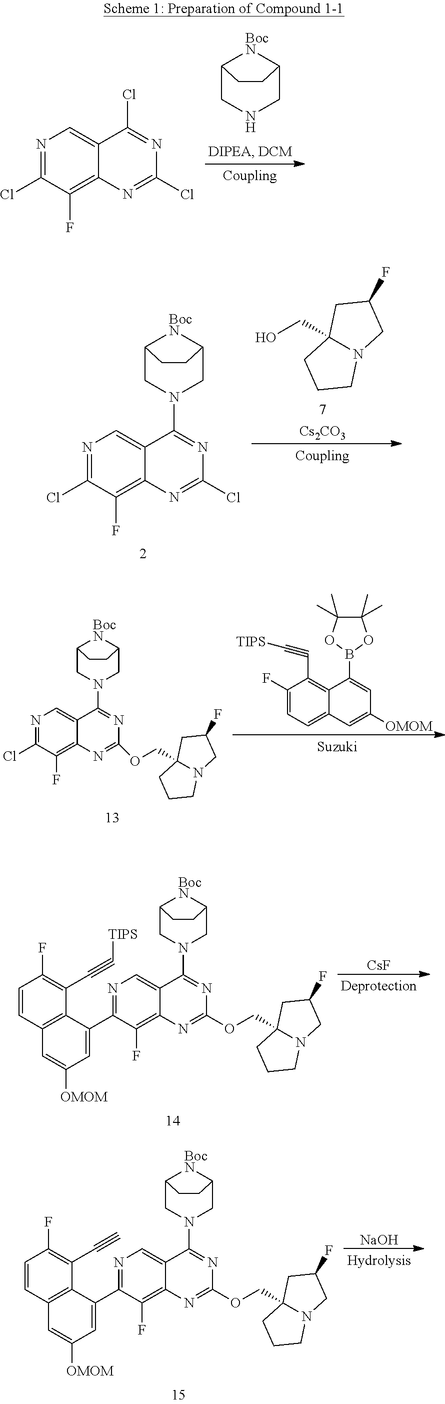

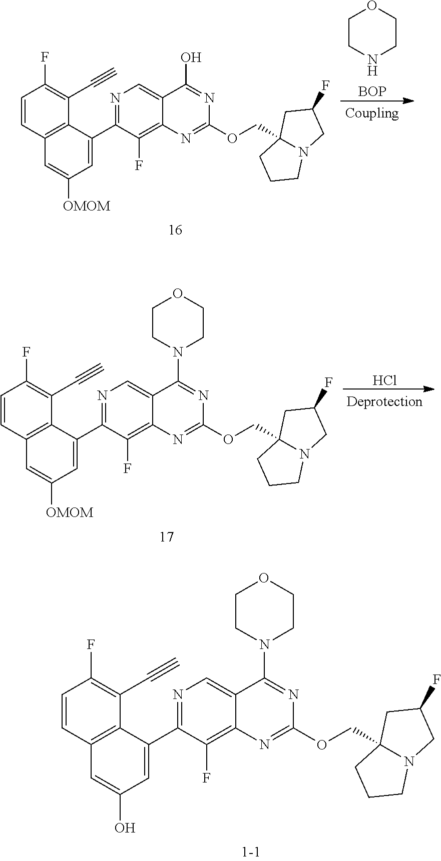

### Preparation of Intermediate 2: tert-butyl 3-{2,7-dichloro-8-fluoropyrido[4,3-d]pyrimidin-4-yl}-3,8-diazabicyclo[3.2.1]octane-8-carboxylate

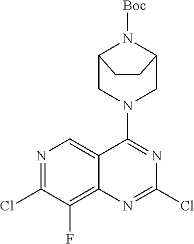 [SMILES: CC(C)(C)OC(=O)N1C2CCC1CN(c1nc(Cl)nc3c(F)c(Cl)ncc13)C2]

To a stirred solution of commercially available 2,4,7-trichloro-8-fluoropyrido[4,3-d]pyrimidine (10 g, 39.6 mmol) in DCM (200 mL) at −40° C. was added DIPEA (20.8 mL, 119 mmol) followed by tert-butyl 3,8-diazabicyclo[3.2.1]octane-8-carboxylate (8.41 g, 39.6 mmol). The reaction mixture was stirred at −40° C. for 30 minutes. The reaction mixture was quenched with water and extracted with DCM. The combined organic layers were washed with brine, dried over anhydrous sodium sulfate, filtered, and evaporated under reduced pressure to provide the crude product. The crude compound was purified by COMBIFLASH™ chromatography (Teledyne ISO, Lincoln, NE) (120 g silica gel column, using 50 to 80% ethyl acetate/petroluem ether) to provide tert-butyl 3-{2,7-dichloro-8-fluoropyrido[4,3-d]pyrimidin-4-yl}-3,8-diazabicyclo[3.2.1]octane-8-carboxylate (13 g, 30.4 mmol, 77% yield) as a pale-yellow solid. MS(ESI) m/z: 428.3 [M+H]⁺.

### Preparation of Intermediate 3: ethyl 2-methylene-5-oxotetrahydro-1H-pyrrolizine-7a(5H)-carboxylate

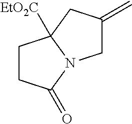

To a stirred solution of ethyl 5-oxopyrrolidine-2-carboxylate (30 g, 191 mmol) and 3-chloro-2-(chloromethyl)prop-1-ene (38.2 g, 305 mmol) in THE (150 mL) at −40° C. under an argon atmosphere was added LiHMDS (382 mL, 382 mmol) dropwise. The reaction mixture was stirred at room temperature for 20 hours. Then, the reaction mixture was poured into aq. 1 M HCl (50 mL) and the pH was adjusted to 7 at 0° C. The mixture was extracted with EtOAc three times. The combined extracts were washed with brine, dried over Na₂SO₄, filtered, and concentrated under reduced pressure to provide a crude residue, which was purified by silica gel column chromatography using COMBIFLASH™ chromatography (Teledyne ISO, Lincoln, NE) (petroluem ether/EtOAc=0 to 50%) to provide the title compound ethyl 2-methylene-5-oxotetrahydro-1H-pyrrolizine-7a(5H)-carboxylate (15 g, 71.7 mmol, 38% yield) as a yellow oil. NMR confirmed the product. ¹H NMR (300 MHz, CDCl₃) δ=5.12-5.01 (m, 2H), 4.32-4.28 (m, 1H), 4.20 (q, J=12.1 Hz, 2H), 3.73 (br d, J=15.5 Hz, 1H), 3.06 (br d, J=15.5 Hz, 1H), 2.88-2.69 (m, 1H), 2.67-2.54 (m, 1H), 2.54-2.38 (m, 2H), 2.21-2.04 (m, 1H), 1.27 (t, J=12.1 Hz, 3H).

### Preparation of Intermediate 4: ethyl 2,5-dioxotetrahydro-1H-pyrrolizine-7a(5H)-carboxylate

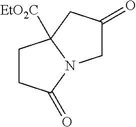 [SMILES: CCOC(=O)C12CCC(=O)N1CC(=O)C2]

A stirred solution of ethyl 2-methylene-5-oxotetrahydro-1H-pyrrolizine-7a(5H)-carboxylate (Intermediate 3, 6 g, 28.7 mmol) in DCM (60 mL)-MeOH (12 mL) was purged with O₃ at −70° C. for 30 minutes. Then N2 was purged for 30 minutes, and dimethyl sulfide (4.5 mL, 57.3 mmol) was added to the mixture at −70° C. The reaction mixture was stirred at room temperature for 16 hours. Then, the reaction mixture was concentrated under reduced pressure to provide a crude residue, which was purified by COMBIFLASH™ chromatography (Teledyne ISO, Lincoln, NE) (120 g column, EtOAc-petroleum ether=0 to 50%) to provide the title compound ethyl 2,5-dioxotetrahydro-1H-pyrrolizine-7a(5H)-carboxylate (4.7 g, 22.25 mmol, 78% yield) as a yellow oil. ¹H NMR (300 MHz, CDCl₃) δ=4.24 (q, J=7.2 Hz, 2H), 4.13 (d, J=18.5 Hz, 1H), 3.57 (d, J=18.5 Hz, 1H), 3.05-2.96 (m, 2H), 2.91-2.72 (m, 1H), 2.54-2.39 (m, 2H), 2.25-2.11 (m, 1H), 1.29 (t, J=7.2 Hz, 3H).

### Preparation of Intermediate 5: (±)-ethyl (2S,7aS)-2-hydroxy-5-oxotetrahydro-1H-pyrrolizine-7a(5H)-carboxylate

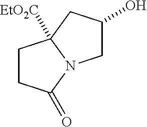 [SMILES: CCOC(=O)[C@@]12CCC(=O)N1C[C@@H](O)C2]

To a stirred solution of ethyl 2,5-dioxotetrahydro-1H-pyrrolizine-7a(5H)-carboxylate (Intermediate 4, 4.7 g, 22.25 mmol) in ethanol (30 mL) at 0° C. was added sodium borohydride (0.25 g, 6.68 mmol), portion wise. The reaction mixture was stirred at 0° C. for 10 minutes. The reaction was quenched with saturated aqueous NH₄Cl (2 mL) and stirred for 30 minutes at 5° C. The reaction mixture was concentrated under reduced pressure to provide a crude residue, which was purified by COMBIFLASH™ chromatography (Teledyne ISO, Lincoln, NE) (40 g column, 0-7% MeOH-DCM) to provide (±) ethyl (2S,7aS)-2-hydroxy-5-oxotetrahydro-1H-pyrrolizine-7a(5H)-carboxylate (2.2 g, 10.32 mmol, 46% yield). ˜10^{%} other diastereomer present. ¹H NMR (300 MHz, CDCl₃) δ=4.71-4.59 (m, 1H), 4.26 (q, J=7.2 Hz, 2H), 3.98 (dd, J=12.8, 5.9 Hz, 1H), 3.12 (d, J=12.8 Hz, 1H), 2.91-2.76 (m, 1H), 2.65-2.39 (m, 4H), 2.16-1.93 (m, 1H), 1.89-1.81 (m, 1H), 1.32 (t, J=7.2 Hz, 3H).

### Preparation of Intermediate 6: ethyl (2R,7aS)-2-fluoro-5-oxotetrahydro-1H-pyrrolizine-7a(5H)-carboxylate

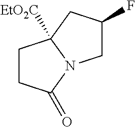 [SMILES: CCOC(=O)[C@@]12CCC(=O)N1C[C@H](F)C2]

To a stirred solution of (1) ethyl (2S,7aS)-2-hydroxy-5-oxotetrahydro-1H-pyrrolizine-7a(5H)-carboxylate (Intermediate 5, 4.5 g, 21.10 mmol) in DCM (30 mL) at −70° C. was added DAST (4.2 mL, 31.7 mmol). The reaction mixture was gradually warmed up to room temperature over a period of 16 h. The reaction mixture was cooled to 0° C. and quenched with MeOH (1.5 mL) and diluted with water (50 mL). The mixture was extracted with DCM (3 times). The combined extracts were washed with brine, dried over Na₂SO₄, filtered, and concentrated under reduced pressure to provide a residue, which was purified by COMBIFLASH™ chromatography (Teledyne ISO, Lincoln, NE) (40 g column, 55% Pet-ether-EtOAc) to provide (1)-ethyl (2R,7aS)-2-fluoro-5-oxotetrahydro-1H-pyrrolizine-7a(5H)-carboxylate (2.1 g, 9.76 mmol, 46.2% yield) as a yellow liquid. The compound was purified by chiral supercritical fluid chromatography (SFC) (column: CHIRALPAK™ IC (Daicel, Japan) (250×21 mm, 5 μm); % CO₂ 70%, % of co-solvent 30%, 0.2% NH₃·H₂O in IPA; Flow: 70 g/min; Back pressure: 100 bar; temp 40° C. Peak 1: retention time=3 min; Peak 2: retention time=4.3 min. Peak 2: ¹H NMR (300 MHz, CDCl₃) δ=5.31 (dt, J=54.2, 4.1 Hz, 1H), 4.30-4.14 (m, 3H), 3.32-3.07 (m, 1H), 2.87-2.56 (m, 3H), 2.50-2.36 (m, 1H), 2.32-2.05 (m, 2H), 1.29 (t, J=7.2 Hz, 3H).

### Preparation of Intermediate 7: ((2R,7aS)-2-fluorotetrahydro-1H-pyrrolizin-7a(5H)-yl)methanol

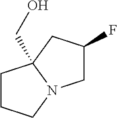 [SMILES: OC[C@@]12CCCN1C[C@H](F)C2]

A solution of ethyl (2R,7aS)-2-fluoro-5-oxotetrahydro-1H-pyrrolizine-7a(5H)-carboxylate (Intermediate 6, 1 g, 4.65 mmol) in THE (9 mL) was added drop wise to an ice-cold solution of 2.4M LAH (2.90 mL, 6.97 mmol) in THF. The reaction mixture was heated to 70° C. for 4 hours. The reaction mixture was cooled to 0° C., quenched with water (1.2 mL), 10% NaOH (3 mL) and additional water (3 mL). Then, the reaction mixture was stirred for 10 minutes and filtered through a diatomaceous earth pad (CELITE™, Sigma Aldrich, St. Louis, MO). The pad was washed with EtOAc. The filtrate was concentrated under reduced pressure and purified by COMBIFLASH™ chromatography (Teledyne ISO, Lincoln, NE) (12 g column, 20 to 60% MeOH-DCM) to provide ((2R,7aS)-2-fluorotetrahydro-1H-pyrrolizin-7a(5H)-yl)methanol (550 mg, 3.45 mmol, 74% yield) as a colorless liquid. TLC system: 20% MeOH-DCM (R^{f}: 0.1); ¹H NMR (300 MHz, CDCl₃) δ=5.29-5.11 (m, 1H), 3.26 (s, 2H), 3.24-3.13 (m, 2H), 3.13-3.01 (m, 1H), 2.98-2.83 (m, 1H), 2.16-2.08 (m, 1H), 2.06-2.01 (m, 1H), 1.99-1.73 (m, 4H). (Exchangeable OH proton not appeared).

### Preparation of Intermediate 8: ethyl (S)-2-((1-phenylethyl)amino)cyclopent-1-ene-1-carboxylate

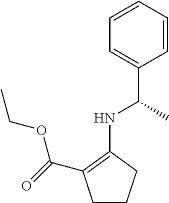 [SMILES: CCOC(=O)C1=C(N[C@@H](C)c2ccccc2)CCC1]

To a stirred solution of ethyl 2-oxocyclopentane-1-carboxylate (140.5 g, 900 mmol) and 4 Å molecular sieves in DCM (500 mL) was added (S)-1-phenylethan-1-amine (109 g, 900 mmol) at room temperature. The reaction mixture was stirred under reflux for 1 day. The reaction mixture was cooled to room temperature, filtered through a diatomaceous earth pad (CELITE™ Sigma Aldrich, St. Louis, MO) and the filtrate was concentrated under reduced pressure to provide a crude residue which was purified by COMBIFLASH™ chromatography (Teledyne ISO, Lincoln, NE) (using 4-5% ethyl acetate/petroleum ether) to provide ethyl (S)-2-((1-phenylethyl)amino)cyclopent-1-ene-1-carboxylate (233 g, 898 mmol, 100% yield). MS(ESI) m/z: 259.8 [M+H]⁺.

### Preparation of Intermediate 9: ethyl (S,E)-1-(3-ethoxy-3-oxopropyl)-2-(((S)-1-phenylethyl)imino)cyclopentane-1-carboxylate

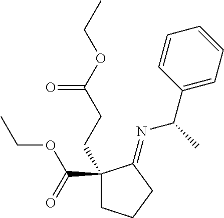 [SMILES: CCOC(=O)CC[C@@]1(C(=O)OCC)CCC/C1=N\[C@@H](C)c1ccccc1]

To a mixture of zinc(II) chloride in 2-MeTHF (473 mL, 898 mmol) and ethyl acrylate (90 g, 898 mmol) at 0° C. was added, dropwise, ethyl (S)-2-((1-phenylethyl)amino)cyclopent-1-ene-1-carboxylate (Intermediate 8, 233 g, 898 mmol) in THE (233 mL) and the mixture was stirred at 0° C. for 16 h. The reaction mixture was neutralized with saturated NaOH solution and extracted with EtOAc (3×500 mL). The combined extracts were dried over anhydrous Na₂SO₄, filtered, and concentrated in vacuo to provide ethyl (S,E)-1-(3-ethoxy-3-oxopropyl)-2-(((S)-1-phenylethyl)imino)cyclopentane-1-carboxylate (300 g, 835 mmol, 93% yield)) as a colorless oil which was taken for the next step without further purification. MS(ESI) m/z: 360.1 [M+H]⁺.

### Preparation of Intermediate 10: ethyl (4aS)-2-oxooctahydro-4aHcyclopenta[b]pyridine-4a-carboxylate

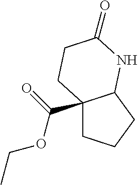

A mixture of ethyl (S,E)-1-(3-ethoxy-3-oxopropyl)-2-(((S)-1-phenylethyl)imino)-cyclopentane-1-carboxylate (Intermediate 9, 145 g, 403 mmol) and 10% palladium on carbon (35 g, 10% w/w) in ethanol (336 mL) was hydrogenated under 50 PSI of hydrogen for 18 h at room temperature. The reaction mixture was filtered through a diatomaceous earth pad (CELITE™ Sigma Aldrich, St. Louis, MO) and the filtrate was concentrated under reduced pressure to provide a crude residue, which was purified by COMBFLASH™ chromatography (Teledyne ISO, Lincoln, NE) (using 4-5% ethyl acetate/petroleum ether) to provide ethyl (4aS)-2-oxooctahydro-4aH-cyclopenta[b]pyridine-4a-carboxylate (36 g, 170 mmol, 42.2% yield) as a colorless oil. MS(ESI) m/z: 211.6 [M+H]⁺.

### Preparation of Intermediate 11: ((4aS,7aR)-octahydro-4aH-cyclopenta[b]pyridin-4a-yl)methanol

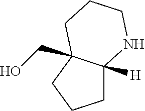 [SMILES: OC[C@@]12CCCN[C@@H]1CCC2]

A solution of ethyl (4aS)-2-oxooctahydro-4aH-cyclopenta[b]pyridine-4a-carboxylate (Intermediate 10, 23.7 g, 112 mmol), in THE (415 mL) was added drop wise to an ice-cold solution of 1M LAH (258 mL, 258 mmol) in THF. The reaction mixture was heated to 70° C. for 4 hours. The reaction mixture was cooled to 0° C., quenched with water (9.8 mL), 10% NaOH (9 mL) and additional water (27 mL). Then, the reaction mixture was allowed to warm to room temperature and stirred for 20 minutes. The reaction mixture was filtered through a diatomaceous earth pad (CELITE™, Sigma Aldrich, St. Louis, MO) and washed with excess EtOAc. The filtrate was dried over Na₂SO₄, filtered, and concentrated under reduced pressure to provide ((4aS,7aR)-octahydro-4aH-cyclopenta[b]pyridin-4a-yl)methanol (17.3 g, 111 mmol, 99% yield) as a white solid which was taken as such for next step without further purification. MS(ESI) m/z: 156.0 [M+H]⁺.

### Preparation of Intermediate 12: ((4aS,7aR)-1-methyloctahydro-4aH-cyclopenta[b]pyridin-4a-yl)methanol

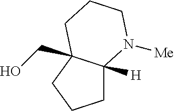 [SMILES: CN1CCC[C@@]2(CO)CCC[C@@H]12]

A solution of ((4aS,7aR)-octahydro-4aH-cyclopenta[b]pyridin-4a-yl)methanol (Intermediate 11, 33.8 g, 218 mmol) in MeOH (335 mL) was cooled to 5° C. and formaldehyde (51.0 mL, 653 mmol) was added over 5 minutes, followed by sodium triacetoxyhydroborate (50.8 g, 239 mmol) in several portions and then stirred at room temperature for 4 h. After 4 h, the reaction mixture was concentrated and diluted with 300 mL of 2-MeTHF, then washed with sat.aq. K₂CO₃ solution. The aqueous phase was extracted with EtOAc (7×), and the combined organic layers were dried over Na₂SO₄, filtered, and concentrated under reduced pressure to provide crude residue. The compound was purified by chiral SFC [(column: BEH 2-ethylpyridine (5×25 cm, 5 m); % CO₂ 90%, % of co-solvent 10%, 0.2% NH40H in MeOH; Flow: 300 mL/min; Back pressure:100 bar; temp 35° C. to provide ((4aS,7aR)-1-methyloctahydro-4aH-cyclopenta[b]pyridin-4a-yl)methanol (28 g, 166 mmol, 76% yield). 1H NMR (400 MHz, CHLOROFORM-d) δ=3.82 (br d, J=4.4 Hz, 1H), 3.70-3.59 (m, 2H), 2.86 (t, J=7.7 Hz, 1H), 2.55-2.46 (m, 1H), 2.41-2.33 (m, 1H), 2.33-2.24 (m, 3H), 1.98-1.83 (m, 2H), 1.83-1.72 (m, 1H), 1.68-1.51 (m, 4H), 1.48-1.33 (m, 3H).

### Preparation of Intermediate 13: tert-butyl 3-(2-{[(2R,7aS)-2-fluoro-hexahydro-1H-pyrrolizin-7a-yl]methoxy}-7-chloro-8-fluoropyrido[4,3-d]pyrimidin-4-yl)-3,8-diazabicyclo[3.2.1]octane-8-carboxylate

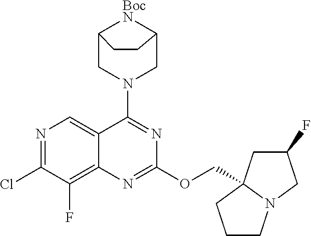 [SMILES: CC(C)(C)OC(=O)N1C2CCC1CN(c1nc(OC[C@@]34CCCN3C[C@H](F)C4)nc3c(F)c(Cl)ncc13)C2]

To a solution of [(2R,7aS)-2-fluoro-hexahydro-1H-pyrrolizin-7a-yl]methanol, HCl salt (Intermediate 7, 5.48 g, 28.0 mmol) in DCM (20 mL) was added approximately 10 g of Na₂CO₃ and the mixture was stirred at room temperature for 30 minutes. The solid was filtered, and the filtrate was evaporated at 20° C. under reduced pressure to provide the free amine, which was dissolved in 1,4-dioxane (100 mL). Then, tert-butyl 3-{2,7-dichloro-8-fluoropyrido[4,3-d]pyrimidin-4-yl}-3,8-diazabicyclo[3.2.1]octane-8-carboxylate (Intermediate 3, 10 g, 23.35 mmol) and Cs₂CO₃ (22.82 g, 70.0 mmol) were added to the above solution and the reaction mixture was heated at 85° C. for 16 h in a sealed tube. The reaction mixture was filtered through a diatomaceous earth pad (CELITE™, Sigma Aldrich, St. Louis, MO), washed with excess DCM and the filtrate was concentrated under reduced pressure to provide a crude residue, which was purified by silica gel column chromatography using COMBFLASH™ chromatography (Teledyne ISO, Lincoln, NE) (120 g REDISEP™ column (Teledyne ISCO, Lincoln, NE), 80 to 100% EtOAc-petroleum ether) to provide tert-butyl 3-(2-{[(2R,7aS)-2-fluoro-hexahydro-1H-pyrrolizin-7a-yl]methoxy}-7-chloro-8-fluoropyrido[4,3-d]pyrimidin-4-yl)-3,8-diazabicyclo[3.2.1]octane-8-carboxylate (7 g, 12.70 mmol, 54.4% yield) as a pale yellow solid. MS(ESI) m/z: 553.5 (M+H)*.

### Preparation of Intermediate 14: tert-butyl 3-(2-{[(2R,7aS)-2-fluoro-hexahydro-1H-pyrrolizin-7a-yl]methoxy}-8-fluoro-7-[7-fluoro-3-(methoxymethoxy)-8-{2-[tris(propan-2-yl)silyl]ethynyl}naphthalen-1-yl]pyrido[4,3-d]pyrimidin-4-yl)-3,8-diazabicyclo[3.2.1]octane-8-carboxylate

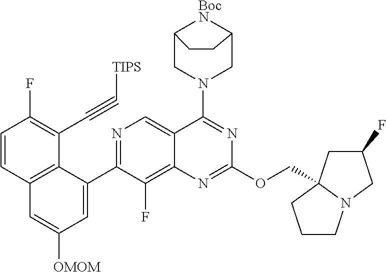

To a stirred solution of tert-butyl 3-(2-{[(2R,7aS)-2-fluoro-hexahydro-1H-pyrrolizin-7a-yl]methoxy}-7-chloro-8-fluoropyrido[4,3-d]pyrimidin-4-yl)-3,8-diazabicyclo[3.2.1]octane-8-carboxylate (Intermediate 13, 5 g, 9.07 mmol) in 1,4-dioxane (110 mL) and water (33 mL) was added ((2-fluoro-6-(methoxymethoxy)-8-(4,4,5,5-tetramethyl-1,3,2-dioxaborolan-2-yl)naphthalen-1-yl)ethynyl)triisopropylsilane (5.12 g, 9.98 mmol) and Cs₂CO₃ (8.87 g, 27.2 mmol) followed by PdCl₂(dppf) (0.66 g, 0.907 mmol). The reaction mixture was purged with N2 for 5 minutes and then heated at 105° C. for 1 h in a microwave reactor. The reaction mixture was cooled to room temperature, diluted with water, and extracted with ethyl acetate. The combined organic layers were washed with brine, dried over anhydrous sodium sulfate, filtered, and concentrated under reduced pressure to provide the crude product, which was purified by COMBIFLASH™ chromatography (Teledyne ISO, Lincoln, NE) (using 80 g silica gel column, using 50 to 100% ethyl acetate/Petroleum ether) to provide tert-butyl 3-(2-{[(2R,7aS)-2-fluoro-hexahydro-1H-pyrrolizin-7a-yl]methoxy}-8-fluoro-7-[7-fluoro-3-(methoxymethoxy)-8-{2-[tris(propan-2-yl)silyl]ethynyl}naphthalen-1-yl]pyrido[4,3-d]pyrimidin-4-yl)-3,8-diazabicyclo[3.2.1]octane-8-carboxylate (3.5 g, 3.88 mmol, 43% yield) as a brown solid. MS(ESI) m/z: 901.7 [M+H]⁺.

### Preparation of Intermediate 15: tert-butyl 3-(2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoropyrido[4,3-d]pyrimidin-4-yl)-3,8-diazabicyclo[3.2.1]octane-8-carboxylate

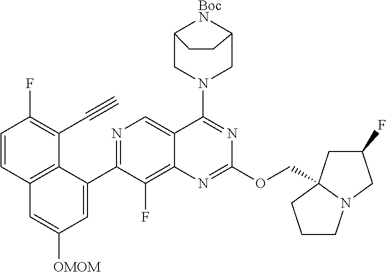

To a stirred solution of tert-butyl 3-(2-{[(2R,7aS)-2-fluoro-hexahydro-1H-pyrrolizin-7a-yl]methoxy}-8-fluoro-7-[7-fluoro-3-(methoxymethoxy)-8-{2-[tris(propan-2-yl)silyl]ethynyl}naphthalen-1-yl]pyrido[4,3-d]pyrimidin-4-yl)-3,8-diazabicyclo[3.2.1]octane-8-carboxylate (Intermediate 14, 3.7 g, 4.11 mmol) in DMF (50 mL) was added CsF (6.24 g, 41.1 mmol). The reaction mixture was heated at 50° C. for 1 h. The reaction mixture was cooled to room temperature, diluted with water, and extracted with ethyl acetate. The combined organic layers were washed with brine, dried over anhydrous sodium sulfate, filtered, and concentrated under reduced pressure to provide tert-butyl 3-(2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoropyrido[4,3-d]pyrimidin-4-yl)-3,8-diazabicyclo[3.2.1]octane-8-carboxylate (2.9 g, 3.89 mmol, 95% yield) as a brown liquid, which was taken for the next step without further purification. MS(ESI) m/z: 746.6 [M+H]⁺.

### Preparation of Intermediate 16: 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoropyrido[4,3-d]pyrimidin-4-ol

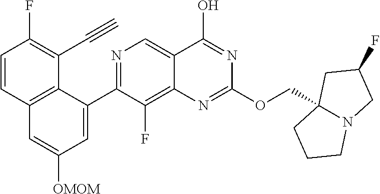

To a stirred solution of tert-butyl 3-(2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoropyrido[4,3-d]pyrimidin-4-yl)-3,8-diazabicyclo[3.2.1]octane-8-carboxylate (Intermediate 15, 3 g, 4.03 mmol) in EtOH (15 mL) and THE (15 mL) was added NaOH (1M aqueous soln.) (40.3 mL, 40.3 mmol). The reaction mixture was heated at 70° C. for 3 h. The reaction mixture was cooled to room temperature, the solvent was evaporated under reduced pressure and the residue was diluted with ethyl acetate. The organic layer was washed with saturated NH₄Cl solution, water, and brine. The organic layer was dried over anhydrous sodium sulfate, filtered, and concentrated under reduced pressure to provide the crude compound, which was purified by COMBIFLASH™ chromatography (Teledyne ISO, Lincoln, NE) (24 g silicagel column, using 10-20% MeOH/DCM) to provide 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoropyrido[4,3-d]pyrimidin-4-ol (850 mg, 1.544 mmol, 38.3% yield) as a brown solid. MS(ESI) m/z: 551.4 [M+H]⁺.

### Preparation of Intermediate 17: (2R,7aS)-7a-[({7-[8-ethynyl-7-fluoro-3-(methoxymethoxy)naphthalen-1-yl]-8-fluoro-4-(morpholin-4-yl)pyrido[4,3-d]pyrimidin-2-yl}oxy)methyl]-2-fluoro-hexahydro-1H-pyrrolizine

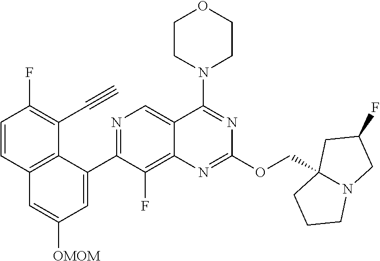

To a stirred solution of 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoropyrido[4,3-d]pyrimidin-4-ol (Intermediate 16, 400 mg, 0.727 mmol) in ACN (10 mL) was added morpholine (76 mg, 0.872 mmol), triethylamine (0.405 mL, 2.91 mmol) and BOP (482 mg, 1.09 mmol). The reaction mixture was heated at 80° C. for 8 h. The reaction mixture was cooled to room temperature, the solvent was evaporated under reduced pressure and the residue was diluted with ethyl acetate. The organic layer was washed with water and brine, and then dried over anhydrous sodium sulfate, filtered, and concentrated under reduced pressure to provide the crude residue. The crude material was purified by COMBIFLASH™ chromatography (Teledyne ISO, Lincoln, NE) (40 g REDISEP™ column (Teledyne ISCO, Lincoln, NE), eluting with a gradient from 60-100% EtOAc in petroleum ether). Fractions containing the desired product were evaporated to provide (2R,7aS)-7a-[({7-[8-ethynyl-7-fluoro-3-(methoxymethoxy)naphthalen-1-yl]-8-fluoro-4-(morpholin-4-yl)pyrido[4,3-d]pyrimidin-2-yl}oxy)methyl]-2-fluoro-hexahydro-1H-pyrrolizine (450 mg, 89% yield) as a brown liquid, which was taken for next step without purification. MS(ESI) m/z: 620.6 [M+H]⁺.

### Example 1-1

### 4-(2-{[(2R,7aS)-2-fluoro-hexahydro-1H-pyrrolizin-7a-yl]methoxy}-8-fluoro-4-(morpholin-4-yl)pyrido[4,3-d]pyrimidin-7-yl)-5-ethynyl-6-fluoronaphthalen-2-ol

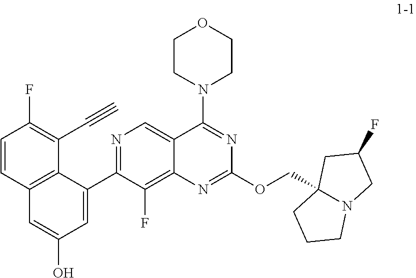 [SMILES: C#Cc1c(F)ccc2cc(O)cc(-c3ncc4c(N5CCOCC5)nc(OC[C@@]56CCCN5C[C@H](F)C6)nc4c3F)c12]

To a stirred solution of tert-butyl 4-(2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoropyrido[4,3-d]pyrimidin-4-yl)-octahydropyrrolo[3,2-b]pyrrole-1-carboxylate (Intermediate 17, 500 mg, 0.807 mmol) in acetonitrile (5 mL) at 0° C. was added 4N HCl (0.49 mL, 16.14 mmol in 1,4-dioxane) and the reaction mixture was stirred at room temperature for 1 h. The volatiles from the reaction mixture were removed under reduced pressure (at lower temperature, ˜30° C.) and the crude residue was co-evaporated with 1,4-dioxane. Then the crude residue was dissolved in 1,4-dioxane (2 mL) and excess TEA (1 mL) was added. The mixture was concentrated under reduced pressure to provide an off-white solid. The crude compound was purified by Prep-HPLC (high performance liquid chromatography) [HPLC Method: Preparative column: EVO (250×30×5), Mobile phase A: 10 mM Ammonium bicarbonate in water-9.5 pH; Mobile phase B: acetonitrile; Gradient=0 (min)−20%, 8-48%, 13.5-48%, 14-100%, 15-100%, 15.5-20-20%; Temperature: 27° C.; Flow rate: 30.0 mL/min; Detection: UV at 220 &254 nm] to provide 4-(2-{[(2R,7aS)-2-fluoro-hexahydro-1H-pyrrolizin-7a-yl]methoxy}-8-fluoro-4-(morpholin-4-yl)pyrido[4,3-d]pyrimidin-7-yl)-5-ethynyl-6-fluoronaphthalen-2-ol (50 mg, 0.084 mmol, 10.4% yield) MS(ESI) m/z: 576.3 [M+H]⁺. ¹H NMR (400 MHz, DMSO-d₆) δ ppm=10.13 (s, 1H), 9.08 (s, 1H), 7.97 (dd, J=5.9, 9.1 Hz, 1H), 7.46 (t, J=9.0 Hz, 1H), 7.39 (d, J=2.5 Hz, 1H), 7.21 (d, J=2.3 Hz, 1H), 5.37-5.19 (m, 1H), 4.15-4.11 (m, 2H), 4.05-3.92 (m, 7H), 3.84-3.82 (m, 4H), 3.15-2.99 (m, 3H), 2.87-2.77 (m, 1H), 2.20-1.98 (m, 2H), 1.93-1.70 (m, 2H).

### Preparation of Intermediate 18: (2R,7aS)-7a-[({7-[8-ethynyl-7-fluoro-3-(methoxymethoxy)naphthalen-1-yl]-8-fluoro-4-(1,4-oxazepan-4-yl)pyrido[4,3-d]pyrimidin-2-yl}oxy)methyl]-2-fluoro-hexahydro-1H-pyrrolizine

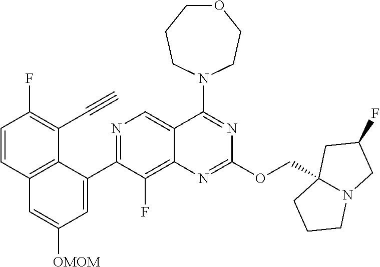

To a stirred solution of 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoropyrido[4,3-d]pyrimidin-4-ol (300 mg, 0.545 mmol) in ACN (10 mL) was added 1,4-oxazepane (66.1 mg, 0.654 mmol), DIPEA (0.29 mL, 1.635 mmol) and BOP (362 mg, 0.817 mmol). The reaction mixture was heated at 80° C. for 8 h. The reaction mixture was cooled to room temperature, the solvent was evaporated under reduced pressure and the residue was diluted with ethyl acetate. The organic layer was washed with water and brine and dried over anhydrous sodium sulfate, filtered, and concentrated under reduced pressure to provide the crude residue. The crude material was purified by COMBIFLASH™ chromatography (Teledyne ISO, Lincoln, NE) (40 g REDISEP™ column (Teledyne ISCO, Lincoln, NE), eluting with a gradient from 60-100% EtOAc in petroleum ether). Fractions containing the desired product were evaporated to provide (2R,7aS)-7a-[({7-[8-ethynyl-7-fluoro-3-(methoxymethoxy)naphthalen-1-yl]-8-fluoro-4-(1,4-oxazepan-4-yl)pyrido[4,3-d]pyrimidin-2-yl}oxy)methyl]-2-fluoro-hexahydro-1H-pyrrolizine (330 mg, 96% yield) as a brown liquid. MS(ESI) m/z: 634.6 [M+H]⁺.

### Example 1-2

### 4-(2-{[(2R,7aS)-2-fluoro-hexahydro-1H-pyrrolizin-7a-yl]methoxy}-8-fluoro-4-(1,4-oxazepan-4-yl)pyrido[4,3-d]pyrimidin-7-yl)-5-ethynyl-6-fluoronaphthalen-2-ol

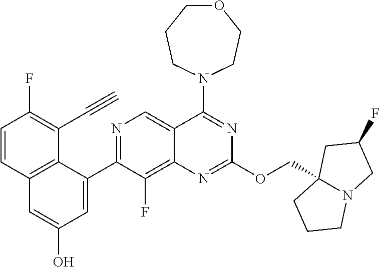 [SMILES: C#Cc1c(F)ccc2cc(O)cc(-c3ncc4c(N5CCCOCC5)nc(OC[C@@]56CCCN5C[C@H](F)C6)nc4c3F)c12]

To a stirred solution of (2R,7aS)-7a-[({7-[8-ethynyl-7-fluoro-3-(methoxymethoxy)naphthalen-1-yl]-8-fluoro-4-(1,4-oxazepan-4-yl)pyrido[4,3-d]pyrimidin-2-yl}oxy)methyl]-2-fluoro-hexahydro-1H-pyrrolizine (330 mg, 0.521 mmol) in acetonitrile (5 mL) at 0° C. was added 4N HCl (1.3 mL, 5.21 mmol in 1,4-dioxane) and the reaction mixture was stirred at room temperature for 1 h. The volatiles from the reaction mixture were removed under reduced pressure (at lower temperature, ˜30° C.) and the crude residue was co-evaporated with 1,4-dioxane. Then the crude residue was dissolved in 1,4-dioxane (2 mL) and excess TEA (1 mL) was added. The mixture was evaporated under reduced pressure to provide an off-white solid. The crude compound was purified by Prep-HPLC [HPLC Method: Preparative Column: Waters XBridge BEH C18 XP(50×2.1 mm) 2.5 m; Mobile Phase A: 5:95 acetonitrile:water with 10 mM NH₄OAc; Mobile Phase B: 95:5 acetonitrile:water with 10 mM NH₄OAc; Temperature: 50° C.; Gradient: 0-100% B over 3 minutes; Flow: 1.1 mL/min. Injection 2 conditions: Column: Waters XBridge BEH C18 XP(50×2.1 mm) 2.5 m; Mobile Phase A: 5:95 acetonitrile:water with 0.1% TFA; Mobile Phase B: 95:5 acetonitrile:water with 0.1% TFA; Temperature:50° C.; Gradient:0-100% B over 3 minutes; Flow: 1.1 mL/min. to provide 4-(2-{[(2R,7aS)-2-fluoro-hexahydro-1H-pyrrolizin-7a-yl]methoxy}-8-fluoro-4-(1,4-oxazepan-4-yl)pyrido[4,3-d]pyrimidin-7-yl)-5-ethynyl-6-fluoronaphthalen-2-ol (28 mg, 0.047 mmol, 9.12% yield) MS(ESI) m/z: 590.4 [M+H]⁺. ¹H NMR (400 MHz, DMSO-d₆) δ ppm=¹H NMR (400 MHz, DMSO-d₆) δ 10.27 (s, 1H), 9.10 (s, 1H), 7.98 (dd, J=9.2, 5.9 Hz, 1H), 7.51-7.36 (m, 2H), 7.18 (d, J=2.4 Hz, 1H), 5.55-5.16 (m, 1H), 4.22-4.01 (m, 6H), 4.00-3.89 (m, 3H), 3.82-3.72 (m, 2H), 3.19-2.96 (m, 3H), 2.88-2.80 (m, 1H), 2.71-2.69 (m, 1H), 2.20-1.96 (m, 4H), 1.90-1.71 (m, 3H).

### Preparation of Intermediate 19: 4-{2,7-dichloro-8-fluoropyrido[4,3-d]pyrimidin-4-yl}-1,4-oxazepane

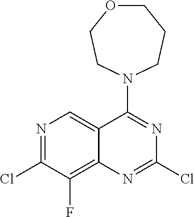 [SMILES: Fc1c(Cl)ncc2c(N3CCCOCC3)nc(Cl)nc12]

To a stirred solution of commercially available 2,4,7-trichloro-8-fluoropyrido[4,3-d]pyrimidine (1 g, 3.96 mmol) in DCM (10 mL) at −40° C. was added DIPEA (1.38 mL, 7.92 mmol) followed by 1,4-oxazepane (0.401 g, 3.96 mmol). The reaction mixture was stirred at −40° C. for 30 minutes. The reaction mixture was quenched with water and extracted with DCM. The combined organic layers were washed with brine, dried over anhydrous sodium sulfate, filtered, and evaporated under reduced pressure to provide the crude product. The crude compound was purified by COMBFLASH™ chromatography (Teledyne ISO, Lincoln, NE) (40 g silica gel column, using 50 to 80% ethyl acetate/petroleum ether) to provide tert-butyl 3-{2,7-dichloro-8-fluoropyrido[4,3-d]pyrimidin-4-yl}-3,8-diazabicyclo[3.2.1]octane-8-carboxylate (0.85 g, 2.68 mmol, 68% yield) as a pale-yellow solid. MS(ESI) m/z: 317.2 [M+H]⁺.

### Preparation of Intermediate 20: 4-(2-{[(4aS,7aR)-1-methyl-octahydro-1H-cyclopenta[b]pyridin-4a-yl]methoxy}-7-chloro-8-fluoropyrido[4,3-d]pyrimidin-4-yl)-1,4-oxazepane

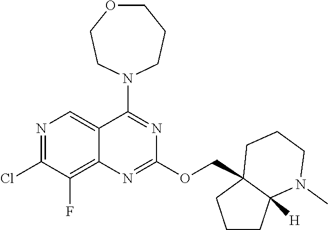 [SMILES: CN1CCC[C@@]2(COc3nc(N4CCCOCC4)c4cnc(Cl)c(F)c4n3)CCC[C@@H]12]

To a stirred solution of ((4aS,7aR)-1-methyloctahydro-4aH-cyclopenta[b]pyridin-4a-yl)methanol (0.48 g, 2.84 mmol) in dry THE (9 mL) was added sodium hydride (0.189 g, 4.73 mmol, 60% w/w) at 0° C. and stirred for 1 h at the same temperature. Then 4-(2,7-dichloro-8-fluoropyrido[4,3-d]pyrimidin-4-yl)-1,4-oxazepane (0.75 g, 2.365 mmol) in THE (2 mL) was added at 0° C. The reaction mixture was allowed to warm to room temperature and was stirred for 1 h. The reaction mixture was quenched with ice cold water and extracted with EtOAc (2×). The combined organic extracts were washed with brine, dried over Na₂SO₄, filtered, and concentrated under reduced pressure to provide crude residue. The crude residue was purified by COMBIFLASH™ chromatography (Teledyne ISO, Lincoln, NE) (40 g silica gel column; 70% to 80% EtOAc in petroleum ether as eluent) to provide the 4-(2-{[(4aS,7aR)-1-methyl-octahydro-1H-cyclopenta[b]pyridin-4a-yl]methoxy}-7-chloro-8-fluoropyrido[4,3-d]pyrimidin-4-yl)-1,4-oxazepane (550 mg, 1.222 mmol, 52% yield) as an off-white solid. MS(ESI) m/z: 450.4 [M+H]⁺.

### Preparation of Intermediate 21: 4-(2-{[(4aS,7aR)-1-methyl-octahydro-1H-cyclopenta[b]pyridin-4a-yl]methoxy}-8-fluoro-7-[7-fluoro-3-(methoxymethoxy)-8-{2-[tris(propan-2-yl)silyl]ethynyl}naphthalen-1-yl]pyrido[4,3-d]pyrimidin-4-yl)-1,4-oxazepane

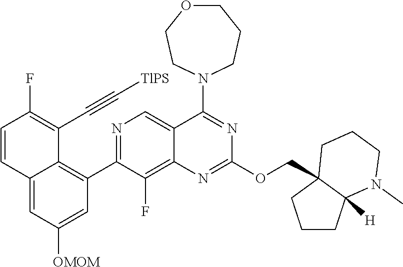

To a stirred solution of 4-(7-chloro-8-fluoro-2-(((4aS,7aR)-1-methyloctahydro-4aH-cyclopenta[b]pyridin-4a-yl)methoxy)pyrido[4,3-d]pyrimidin-4-yl)-1,4-oxazepane (500 mg, 1.111 mmol) in 1,4-dioxane (26 mL), H₂O (7.5 mL) were added ((2-fluoro-6-(methoxymethoxy)-8-(4,4,5,5-tetramethyl-1,3,2-dioxaborolan-2-yl)naphthalen-1-yl)ethynyl)triisopropylsilane (627 mg, 1.222 mmol), and Cs₂CO₃ (1086 mg, 3.33 mmol). The reaction mixture was purged with argon for 5 minutes and PdCl₂(dppf) (81 mg, 0.111 mmol) was added. The reaction mixture was again purged with argon for 3 minutes and then heated at 100° C. for 1 h in a microwave reactor. The reaction mixture was cooled to room temperature, diluted with water, and extracted with ethyl acetate. The combined organic layers were washed with brine, dried over anhydrous sodium sulfate, filtered, and concentrated under reduced pressure to provide the crude product, which was purified by COMBIFLASH™ chromatography (Teledyne ISO, Lincoln, NE) (using 40 g silica gel column, using 50 to 100% ethyl acetate/petroleum ether) to provide 4-(2-{[(4aS,7aR)-1-methyl-octahydro-1H-cyclopenta[b]pyridin-4a-yl]methoxy}-8-fluoro-7-[7-fluoro-3-(methoxymethoxy)-8-{2-[tris(propan-2-yl)silyl]ethynyl}naphthalen-1-yl]pyrido[4,3-d]pyrimidin-4-yl)-1,4-oxazepane (500 mg, 0.625 mmol, 56% yield) as brown solid. MS(ESI) m/z: 800.4 [M+H]⁺.

### Preparation of Intermediate 22: 4-(2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoropyrido[4,3-d]pyrimidin-4-yl)-1,4-oxazepane

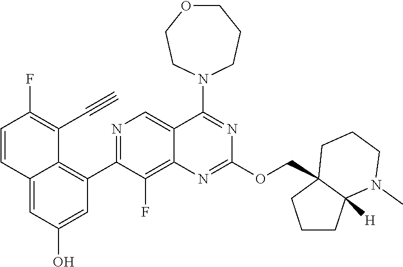 [SMILES: C#Cc1c(F)ccc2cc(O)cc(-c3ncc4c(N5CCCOCC5)nc(OC[C@]56CCC[C@H]5N(C)CCC6)nc4c3F)c12]

To a stirred solution of 4-(8-fluoro-7-(7-fluoro-3-(methoxymethoxy)-8-((triisopropylsilyl)ethynyl)naphthalen-1-yl)-2-(((4aS,7aR)-1-methyloctahydro-4aH-cyclopenta[b]pyridin-4a-yl)methoxy)pyrido[4,3-d]pyrimidin-4-yl)-1,4-oxazepane (0.5 g, 0.625 mmol) in DMF (5 mL) was added CsF (0.95 g, 41.1 mmol). The reaction mixture was heated at 50° C. for 1 h. The reaction mixture was cooled to room temperature, diluted with water, and extracted with ethyl acetate. The combined organic layers were washed with brine, dried over anhydrous sodium sulfate, filtered, and concentrated under reduced pressure to provide 4-(2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoropyrido[4,3-d]pyrimidin-4-yl)-1,4-oxazepane as a brown liquid, which was taken for next step without further purification. MS(ESI) m/z: 644.3 [M+H]⁺.

### Example 1-3

### 4-(2-{[(4aS,7aR)-1-methyl-octahydro-1H-cyclopenta[b]pyridin-4a-yl]methoxy}-8-fluoro-4-(1,4-oxazepan-4-yl)pyrido[4,3-d]pyrimidin-7-yl)-5-ethynyl-6-fluoronaphthalen-2-ol

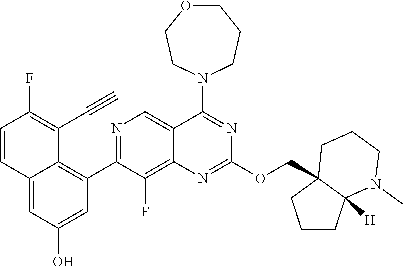 [SMILES: C#Cc1c(F)ccc2cc(O)cc(-c3ncc4c(N5CCCOCC5)nc(OC[C@]56CCC[C@H]5N(C)CCC6)nc4c3F)c12]

To a stirred solution of 4-(8-fluoro-7-(7-fluoro-3-(methoxymethoxy)-8-((triisopropylsilyl)ethynyl)naphthalen-1-yl)-2-(((4aS,7aR)-1-methyloctahydro-4aH-cyclopenta[b]pyridin-4a-yl)methoxy)pyrido[4,3-d]pyrimidin-4-yl)-1,4-oxazepane (450 mg, 0.562 mmol) in acetonitrile (5 mL) at 0° C. was added HCl (4M in dioxane) (0.703 mL, 2.81 mmol) and the reaction mixture was stirred at room temperature for 1 h. The volatiles from the reaction mixture were removed under reduced pressure (at lower temperature, ˜30° C.) and the crude residue was co-evaporated with 1,4-dioxane. Then the crude residue was dissolved in 1,4-dioxane (2 mL) and excess TEA (1 mL) was added. The mixture was evaporated under reduced pressure to provide an off-white solid. The crude compound was purified by Prep-HPLC [HPLC Method: Preparative column: YMC ExRS (20×250) 5u, Mobile phase A: 10 mM ammonium bicarbonate in water-9.5 pH; Mobile phase B: acetonitrile; Gradient=0 (min)−50%, 2-50%, 15-70%, 16-70%, 17-100%; Temperature: 27° C.; Flow rate: 19 mL/min; Detection: UV at 220 &254 nm] to provide 4-(2-{[(4aS,7aR)-1-methyl-octahydro-1H-cyclopenta[b]pyridin-4a-yl]methoxy}-8-fluoro-4-(1,4-oxazepan-4-yl)pyrido[4,3-d]pyrimidin-7-yl)-5-ethynyl-6-fluoronaphthalen-2-ol (78 mg, 0.127 mmol, 23% yield). MS(ESI) m/z: 600.4 [M+H]⁺. 1H NMR (400 MHz, DMSO-d₆) δ ppm=10.16 (s, 1H), 9.10 (s, 1H), 7.98 (dd, J=9.2, 5.9 Hz, 1H), 7.47 (t, J=9.0 Hz, 1H), 7.40 (d, J=2.5 Hz, 1H), 7.18 (d, J=2.4 Hz, 1H), 4.46 (d, J=10.6 Hz, 1H), 4.23-4.07 (m, 5H), 4.03-3.91 (m, 3H), 3.81-3.73 (m, 2H), 2.72-2.63 (m, 1H), 2.69-2.59 (m, 1H), 2.25-2.14 (m, 4H), 2.14-2.06 (m, 3H), 1.92-1.65 (m, 3H), 1.65-1.49 (m, 6H), 1.47-1.33 (m, 1H).

The examples in Table 1 were prepared according to procedures described for Example 1-1 from appropriate starting materials.

Examples 1-22/1-23, 1-25/1-26, 1-42/1-43, 1-44/1-45, 1-60/1-61, 1-62/1-63, 1-69/1-70, 1-71/1-72, 1-73/1-74, 1-112/1-113, 1-119/1-140, 120/121, 1-124, 1-125, 1-126/1-146, 1-128, 1-130/1-131, 1-132/1-133, 1-135/1-143, 1-136, 1-139, 1-144 were synthesized as diastereomeric mixtures. Final compounds were purified by Prep-HPLC to provide pure diastereomeric mixtures which were further purified by either chiral HPLC or SFC as noted to provide single diastereomers. In the case of Example 2-40/2-41, commercially available 3-cyanopiperidine was subjected to chiral SFC separation and in case of examples 1-114, 1-116, 1-117, 1-118, 1-138, 1-140, and 1-142 racemic fragments were synthesized and then subjected to chiral SFC separation followed at the start of the reaction sequence to provide a single diastereomer. Superscripts appearing after the example number in the table designate which of the following chiral separation conditions were used:
A) CHIRALCEL™ OJ-H (Daicel, Japan) (250×21) mm, 5 μm; % CO₂: 70%; % Co solvent: 30% 0.2% DEA in IPA; Total Flow: 80.0 g/min; Back Pressure: 100bar; Temperature: 35° C.; UV: 240 nm
B) CHIRALPAK™ IC (Daicel, Japan) (250×21) mm, 5 μm; % CO₂: 75%; % Co solvent: 25% 0.2% IPA in methanol; Total Flow: 90.0 g/min; Back Pressure: 100bar; Temperature: 35° C.; UV: 220 nm
C) CHIRALPAK™ IE (Daicel, Japan) (250×30) mm, 5 μm; % CO₂: 50%; % Co solvent: 50% 0.2% IPA in methanol; Total Flow: 120.0 g/min; Back Pressure: 100bar; Temperature: 35° C.; UV: 254 nm
D) CHIRALPAK™ IE (Daicel, Japan) (250×30) mm, 5 μm; % CO₂: 70%; % Co solvent: 30% 0.2% IPA IN methanol; Total Flow: 150.0 g/min; Back Pressure: 100bar; Temperature: 35° C.; UV: 220 nm
E) CHIRALPAK™ IE (Daicel, Japan) (250×30) mm, 5 μm; Mobile Phase A: Mobile Phase B: 10 mM ammonium acetate in methanol:0.07% acetic acid; Flow rate: 30.0 ml/min; Gradient: T/% B:0/100; 32/100
F) WHELK-O™ 1 (R,R) (Regis Technologies, Morton Grove, IL) (250×21) mm, 5 μm; % CO₂: 70%; % Co solvent: 30% of 0.2% DEA in ACN/IPA; Total Flow: 90.0 g/min; Back Pressure: 100bar; Temperature: 35° C.; UV: 220 nm
G) LUX™ Cellulose-2 (Phenomenex, Torrance, CA) (250×21.5) mm, 5 μm; % CO₂: 60%; % Co solvent: 40% of 5 mM ammonium acetate in ACN:MEOH (50:50); Total Flow: 80.0 g/min; Back Pressure: 100bar; Temperature: 35° C.; UV: 220 nm
H) LUX™ Cellulose-1 (Phenomenex, Torrance, CA) (250×21.5) mm, 5 μm; % CO₂: 50%; % Co solvent: 50% of 5 mM ammonium acetate MEOH; Total Flow: 80.0 g/min; Back Pressure: 100bar; Temperature: 35° C.; UV: 220 nm
I) LUX™ Cellulose-4 (Phenomenex, Torrance, CA) (250×21.5) mm, 5 μm; % CO₂: 50%; % Co solvent: 50% of 5 mM ammonium MEOH; Total Flow: 70.0 g/min; Back Pressure: 100bar; Temperature: 35° C.; UV: 220 nm
J) CHIRALPAK™ IG (Daicel, Japan) (250×21) mm, 5 μm; % CO₂: 75%; % Co solvent: 25% 0.2% ammonia in methanol; Total Flow: 90.0 g/min; Back Pressure: 100bar; Temperature: 35° C.; UV: 220 nm
K) CHIRALCEL™ OD-H (Daicel, Japan) (250×21) mm, 5 μm; % CO₂: 80%; % Co solvent: 20% 0.2% ammonia in methanol; Total Flow: 80.0 g/min; Back Pressure: 100bar; Temperature: 35° C.; UV: 240 nm

TABLE 1

| | | | LCMS | |
|---|---|---|---|---|
| # | Structure | IUPAC | (M + H)⁺ | ¹H NMR |
| 1-4 | 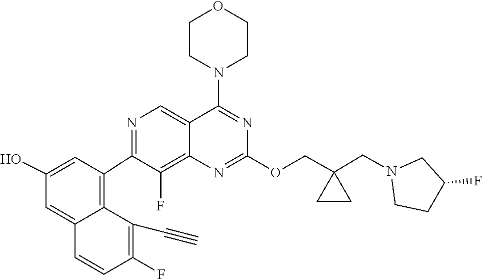 [SMILES: C#Cc1c(F)ccc2cc(O)cc(-c3ncc4c(N5CCOCC5)nc(OCC5(CN6CC[C@@H](F)C6)CC5)nc4c3F)c12] | 5-ethynyl-6-fluoro- 4-{8-fluoro-2-[(1- {[(3R)-3- fluoropyrrolidin-1- yl]methyl} cyclopropyl) methoxy]-4- (morpholin-4- yl)pyrido[4,3- d]pyrimidin-7- yl}naphthalen-2-ol | 590.5 | ¹H NMR (400 MHz, DMSO-d₆) δ ppm = 10.23-10.13 (m, 1H), 9.07 (s, 1H), 7.98 (dd, J = 9.3, 6.0 Hz, 1H), 7.50-7.44 (m, 1H), 7.40 (d, J = 2.5 Hz, 1H), 7.20 (d, J = 2.5 Hz, 1H), 5.28-5.07 (m, 1H), 4.35- 4.21 (m, 2H), 4.08-3.97 (m, 2H), 3.96-3.88 (m, 2H), 3.86-3.79 (m, 5H), 2.91-2.76 (m, 2H), 2.70- 2.55 (m, 1H), 2.47-2.38 (m, 2H), 2.36-2.28 (m, 1H), 2.19-2.03 (m, 1H), |
| | | | | 1.93-1.76 (m, 1H), 0.64 |
| | | | | (m, 2H), 0.48-0.42 (m, |
| | | | | 2H) |
| 1-5 | 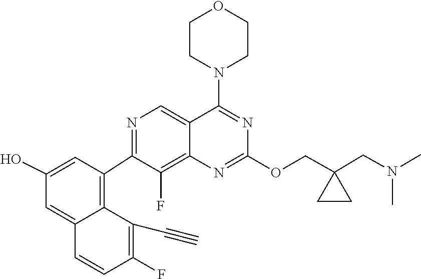 [SMILES: C#Cc1c(F)ccc2cc(O)cc(-c3ncc4c(N5CCOCC5)nc(OCC5(CN(C)C)CC5)nc4c3F)c12] | 4-[2-({1- [(dimethylamino) methyl]cyclopropyl} methoxy)-8-fluoro- 4-(morpholin-4- yl)pyrido[4,3- d]pyrimidin-7-yl]- 5-ethynyl-6- fluoronaphthalen- 2-ol | 546.2 | ¹H NMR (400 MHz, DMSO-d₆) δ ppm = 10.17 (dd, J = 5.0, 3.0 Hz, 1H), 9.11-9.06 (m, 1H), 8.02- 7.95 (m, 1H), 7.51-7.39 (m, 2H), 7.20 (d, J = 2.8 Hz, 1H), 4.28-4.24 (m, 2H), 4.04-3.90 (m, 4H), 3.83-3.81 (m, 3H), 2.53 (d, J = 2.0 Hz, 6H), 2.28-2.16 (m, 4H), 0.70-0.64 (m, 2H), 0.51-0.39 (m, 2H) |
| 1-6 | 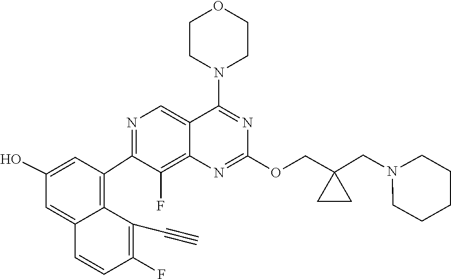 [SMILES: C#Cc1c(F)ccc2cc(O)cc(-c3ncc4c(N5CCOCC5)nc(OCC5(CN6CCCCC6)CC5)nc4c3F)c12] | 5-ethynyl-6-fluoro- 4-[8-fluoro-4- (morpholin-4-yl)-2- ({1-[(piperidin-1- yl)methyl] cyclopropyl} methoxy)pyrido [4,3-d]pyrimidin- 7-yl]naphthalen-2- ol | 586.1 | ¹H NMR (400 MHz, DMSO-d₆) δ ppm = 9.09 (s, 1H), 7.98 (dd, J = 5.9, 8.9 Hz, 1H), 7.52-7.43 (m, 1H), 7.40 (d, J = 2.8 Hz, 1H), 7.19 (d, J = 2.8 Hz, 1H), 4.30 (s, 2H), 4.07- 3.99 (m, 2H), 3.97-3.90 (m, 3H), 3.85-3.79 (m, 5H), 3.18 (s, 2H) 2.11 (s, 2H), 1.67-1.36 (m, 4H), 1.24 (s, 2H), 0.94-0.81 (m, 1H), 0.79-0.66 (m, 2H), 0.59-0.39 (m, 2H). |
| 1-7 | 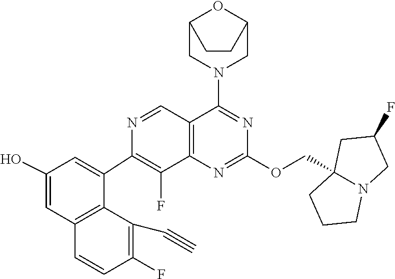 [SMILES: C#Cc1c(F)ccc2cc(O)cc(-c3ncc4c(N5CC6CCC(C5)O6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{8-oxa-3- azabicyclo[3.2.1] octan-3- yl}pyrido[4,3- d]pyrimidin-7-yl)- 5-ethynyl-6- fluoronaphthalen-2- ol | 602.3 | ¹H NMR (400 MHz, DMSO-d₆) δ ppm = 10.16 (s, 1H), 9.08 (s, 1H), 7.98 (dd, J = 9.5, 6.0 Hz, 1H), 7.47 (t, J = 9.1 Hz, 1H), 7.40 (d, J = 2.5 Hz, 1H), 7.18 (d, J = 2.8 Hz, 1H), 5.46-5.21 (m, 1H), 4.53- 4.44 (m, 3H), 4.34-4.27 (m, 1H), 4.15 (s, 1H), 3.92 (d, J = 7.8 Hz, 1H), 3.79- 3.65 (m, 2H), 3.29-3.27 (m, 2H), 3.21-3.11 (m, 2H), 2.53-2.52 (m, 1H), 2.13-2.06 (m, 2H), 1.93- 1.77 (m, 7H), 1.24 (s, 1H) |
| 1-8 | 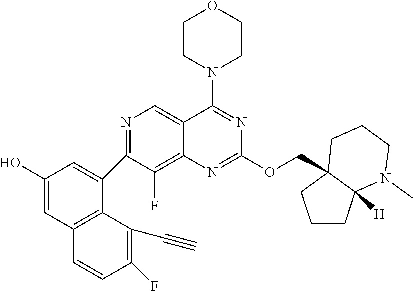 [SMILES: C#Cc1c(F)ccc2cc(O)cc(-c3ncc4c(N5CCOCC5)nc(OC[C@]56CCC[C@H]5N(C)CCC6)nc4c3F)c12] | 4-(2-{[(4aS,7aR)-1- methyl-octahydro- 1H- cyclopenta[b] pyridin-4a- yl]methoxy}- 8-fluoro-4- (morpholin-4- yl)pyrido[4,3- d]pyrimidin-7-yl)- 5-ethynyl-6- fluoronaphthalen-2- ol | 586.3 | ¹H NMR (400 MHz, DMSO-d₆) δ ppm = 9.07 (s, 1H), 7.95 (dd, J = 8.9, 6.4 Hz, 1H), 7.45 (t, J = 8.9 Hz, 1H), 7.39 (d, J = 2.5 Hz, 1H), 7.21 (d, J = 2.8 Hz, 1H), 4.48 (d, J = 10.6 Hz, 1H), 4.18-4.11 (m, 1H), 4.04-3.89 (m, 6H), 3.85- 3.79 (m, 4H), 2.24-2.20 (m, 1H), 2.17 (s, 3H), 2.08- 2.03 (m, 2H), 1.63-1.54 (m, 4H), 1.43-1.37 (m, 2H), 1.26-1.22 (m, 3H). |
| 1-9 | 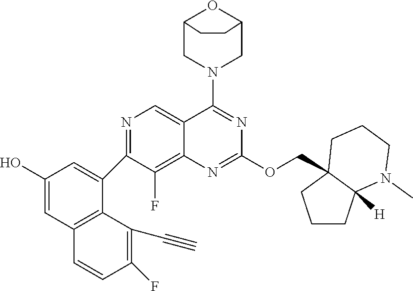 [SMILES: C#Cc1c(F)ccc2cc(O)cc(-c3ncc4c(N5CC6CCC(C5)O6)nc(OC[C@]56CCC[C@H]5N(C)CCC6)nc4c3F)c12] | 4-(2-{[(4aS,7aR)-1- methyl-octahydro- 1H- cyclopenta[b] pyridin-4a- yl]methoxy}- 8-fluoro-4- {8-oxa-3- azabicyclo[3.2.1] octan-3- yl}pyrido[4,3- d]pyrimidin-7-yl)- 5-ethynyl-6- fluoronaphthalen- 2-ol | 612.3 | ¹H NMR (400 MHz, DMSO-d₆) δ ppm = 10.16 (br d, J = 1.0 Hz, 1H), 9.06 (s, 1H), 7.99-7.95 (m, 1H), 7.47 (t, J = 9.1 Hz, 1H), 7.40 (d, J = 2.5 Hz, 1H), 7.18 (d, J = 2.4 Hz, 1H), 4.51-4.41 (m, 4H), 4.27 (br dd, J = 12.3, 7.9 Hz, 1H), 4.14 (d, J = 10.8 Hz, 1H), 3.95 (d, J = 3.5 Hz, 1H), 3.77-3.62 (m, 2H), 2.28-2.16 (m, 5H), 1.88- 1.80 (m, 4H), 1.75-1.36 (m, 10H), 1.27-1.22 (m, 1H) |
| 1-10 | 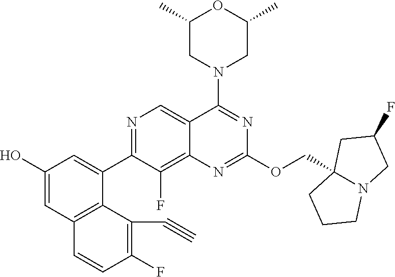 [SMILES: C#Cc1c(F)ccc2cc(O)cc(-c3ncc4c(N5C[C@@H](C)O[C@@H](C)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy }-4- [(2R,6S)-2,6- dimethylmorpholin- 4-yl]-8- fluoropyrido[4,3- d]pyrimidin-7-yl)- 5-ethynyl-6- fluoronaphthalen- 2-ol | 604.3 | ¹H NMR (400 MHz, DMSO-d₆) δ ppm = 9.08 (s, 1H), 7.98 (dd, J = 6.0, 9.3 Hz, 1H), 7.47 (t, J = 9.0 Hz, 1H), 7.40 (d, J = 2.5 Hz, 1H), 7.20 (d, J = 2.3 Hz, 1H), 5.39-5.16 (m, 1H), 4.44 (br d, J = 13.3 Hz, 1H), 4.40-4.33 (m, 1H), 4.21-4.09 (m, 1H), 4.04 (dd, J = 7.4, 10.4 Hz, 1H), 3.98 (d, J = 0.8 Hz, 1H), 3.88-3.73 (m, 2H), 3.13- 3.00 (m, 5H), 2.90-2.77 (m, 1H), 2.18-2.11 (m, 1H), 2.10-1.98 (m, 3H), 1.92-1.70 (m, 3H), 1.20 (dd, J = 3.3, 6.0 Hz, 5H). |
| 1-11 | 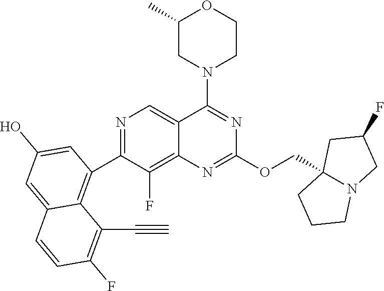 [SMILES: C#Cc1c(F)ccc2cc(O)cc(-c3ncc4c(N5CCO[C@@H](C)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(2S)-2- methylmorpholin-4- yl]pyrido[4,3- d]pyrimidin-7-yl)- 5-ethynyl-6- fluoronaphthalen- 2-ol | 590.2 | ¹H NMR (400 MHz, DMSO-d₆) δ ppm = 10.17 (s, 1H), 9.11-9.09 (m, 1H), 7.98 (dd, J = 9.1, 5.9 Hz, 1H), 7.46 (t, J = 9.0 Hz, 1H), 7.40 (d, J = 2.6 Hz, 1H), 7.19 (d, J = 2.4 Hz, 1H), 4.49-4.42 (m, 1H), 4.40-4.21 (m, 2H), 4.02- 3.95 (m, 2H), 3.90 (s, 1H), 3.81-3.68 (m, 3H), 3.61- 3.40 (m, 3H), 3.28-3.14 (m, 3H), 2.54-2.52 (m, 1H), 2.31-2.05 (m, 2H), 2.03-1.82 (m, 3H), 1.25- 1.17 (m, 3H). |
| 1-12 | 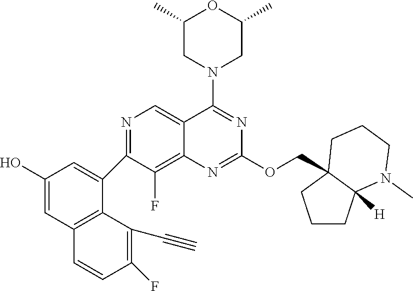 [SMILES: C#Cc1c(F)ccc2cc(O)cc(-c3ncc4c(N5C[C@@H](C)O[C@@H](C)C5)nc(OC[C@]56CCC[C@H]5N(C)CCC6)nc4c3F)c12] | 4-(2-{[(4aS,7aR)-1- methyl-octahydro- 1H- cyclopenta[b] pyridin-4a- yl]methoxy}- 4-[(2R,6S)-2,6- dimethylmorpholin- 4-yl]-8- fluoropyrido[4,3- d]pyrimidin-7-yl)- 5-ethynyl-6- fluoronaphthalen- 2-ol | 614.3 | ¹H NMR (400 MHz, DMSO-d₆) δ ppm = 10.19 (br d, J =1.3 Hz, 1H), 9.08 (s, 1H), 7.98 (dd, J = 9.0, 5.9 Hz, 1H), 7.47 (t, J = 8.9 Hz, 1H), 7.40 (d, J = 2.4 Hz, 1H), 7.20 (d, J = 2.4 Hz, 1H), 4.55-4.33 (m, 3H), 4.21-4.13 (m, 1H), 3.99 (d, J = 3.9 Hz, 1H), 3.89-3.77 (m, 2H), 3.14-3.04 (m, 2H), 2.81- 2.70 (m, 1H), 2.32-2.20 (m, 4H), 2.11 (s, 1H), 1.88-1.81 (m, 1H), 1.77-1.70 (m, 1H), 1.69- 1.52 (m, 6H), 1.46-1.40 (m, 1H), 1.27-1.18 (m, 7H). |
| 1-13 | 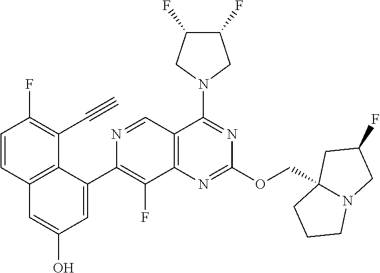 [SMILES: C#Cc1c(F)ccc2cc(O)cc(-c3ncc4c(N5C[C@@H](F)[C@@H](F)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4- [(3R,4S)-3,4- difluoropyrrolidin- 1-yl]-8- fluoropyrido[4,3- d]pyrimidin-7-yl)- 5-ethynyl-6- fluoronaphthalen- 2-ol | 596.2 | |
| 1-14 | 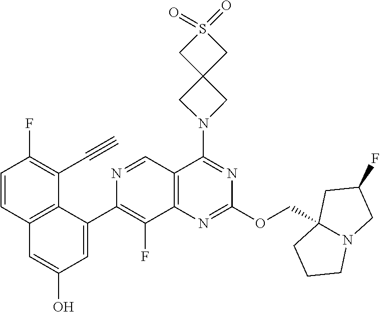 [SMILES: C#Cc1c(F)ccc2cc(O)cc(-c3ncc4c(N5CC6(C5)CS(=O)(=O)C6)nc(OC[C@@]56CCCN5C[C@H](F)C6)nc4c3F)c12] | 6-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 2¿6-thia-6- azaspiro[3.3] heptane-2,2-dione | 636.2 | |
| 1-15 | 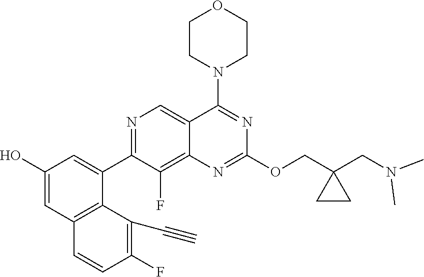 [SMILES: C#Cc1c(F)ccc2cc(O)cc(-c3ncc4c(N5CCOCC5)nc(OCC5(CN(C)C)CC5)nc4c3F)c12] | 4-[2-({1- [(dimethylamino) methyl]cyclopropyl} methoxy)-8-fluoro- 4-(morpholin-4- yl)pyrido[4,3- d]pyrimidin-7-yl]- 5-ethynyl-6- fluoronaphthalen-2- ol | 546.3 | |
| 1-16 | 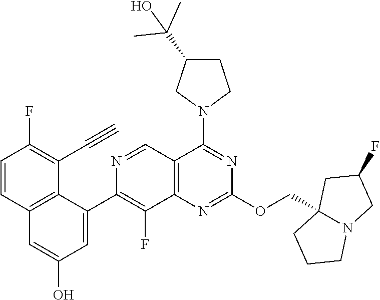 [SMILES: C#Cc1c(F)ccc2cc(O)cc(-c3ncc4c(N5CC[C@@H](C(C)(C)O)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(3R)-3-(2- hydroxypropan-2- yl)pyrrolidin-1- yl]pyrido[4,3- d]pyrimidin-7-yl)- 5-ethynyl-6- fluoronaphthalen-2- ol | 618.1 | |
| 1-17 | 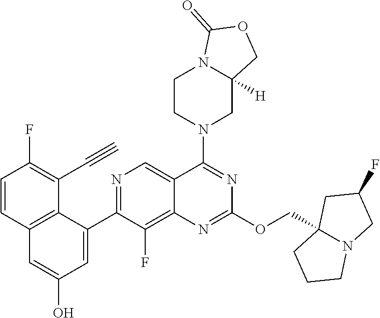 [SMILES: C#Cc1c(F)ccc2cc(O)cc(-c3ncc4c(N5CCN6C(=O)OC[C@H]6C5)nc(OC[C@@]56CCCN5C[C@H](F)C6)nc4c3F)c12] | (8aR)-7-(2- {[(2R,7aS)-2- fluoro-hexahydro- 1H-pyrrolizin-7a- yl]methoxy }-7-(8- ethynyl-7-fluoro-3- hydroxynaphthalen- 1-yl)-8- fluoropyrido[4,3- d]pyrimidin-4-yl)- hexahydro-1H- [1,3]oxazolo[3,4- a]piperazin-3-one | 631.2 | |
| 1-18 | 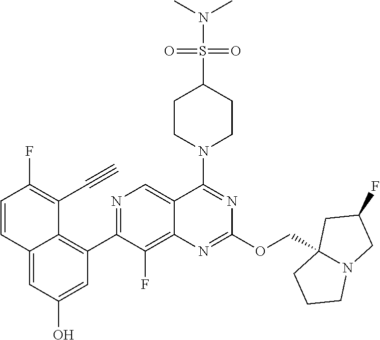 [SMILES: C#Cc1c(F)ccc2cc(O)cc(-c3ncc4c(N5CCC(S(=O)(=O)N(C)C)CC5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- N,N- dimethylpiperidine- 4-sulfonamide | 681.3 | |
| 1-19 | 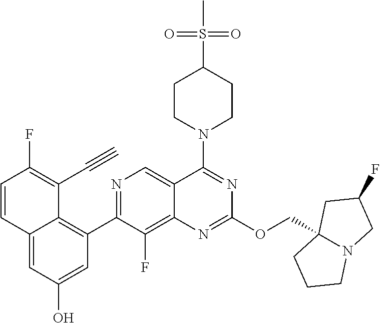 [SMILES: C#Cc1c(F)ccc2cc(O)cc(-c3ncc4c(N5CCC(S(C)(=O)=O)CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-(4- methanesulfonyl- piperidin-1- yl)pyrido[4,3- d]pyrimidin-7-yl)- 5-ethynyl-6- fluoronaphthalen-2- ol | 652.2 | |
| 1-20 | 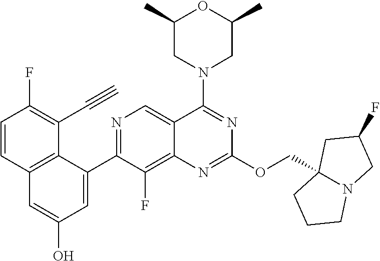 [SMILES: C#Cc1c(F)ccc2cc(O)cc(-c3ncc4c(N5C[C@@H](C)O[C@@H](C)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4- [(2R,6S)-2,6- dimethylmorpholin- 4-yl]-8- fluoropyrido[4,3- d]pyrimidin-7-yl)- 5-ethynyl-6- fluoronaphthalen-2- ol | 604.3 | |
| 1-21 | 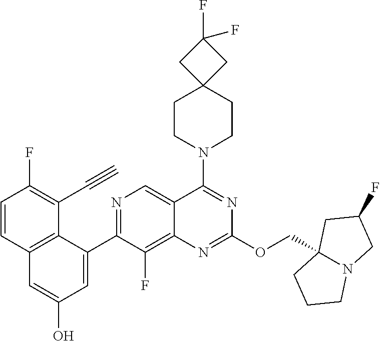 [SMILES: C#Cc1c(F)ccc2cc(O)cc(-c3ncc4c(N5CCC6(CC5)CC(F)(F)C6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4- {2,2-difluoro-7- azaspiro[3.5] nonan-7-yl}-8- fluoropyrido[4,3- d]pyrimidin-7-yl)- 5-ethynyl-6- fluoronaphthalen-2- ol | 650.3 | |
| 1-22 | 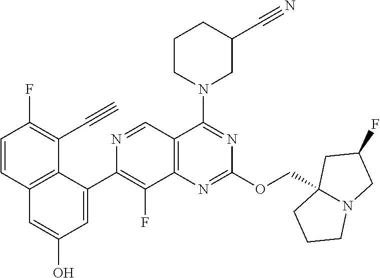 [SMILES: C#Cc1c(F)ccc2cc(O)cc(-c3ncc4c(N5CCCC(C#N)C5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4- yl)piperidine-3- carbonitrile | 599.3 | ¹H NMR (400 MHz, DMSO-d₆) δ ppm = 10.18 (d, J = 4.1 Hz, 1H), 9.09 (d, J = 5.4 Hz, 1H), 8.02-7.95 (m, 1H), 7.47 (t, J = 8.9 Hz, 1H), 7.41 (s, 1H), 7.22 (dd, J = 4.2, 2.7 Hz, 1H), 5.46- 5.24 (m, 1H), 4.41-3.91 (m, 6H), 3.87-3.71 (m, 2H), 3.26-3.07 (m, 3H), 3.01-2.86 (m, 1H), 2.28- 1.75 (m, 10H) |
| 1-23 | 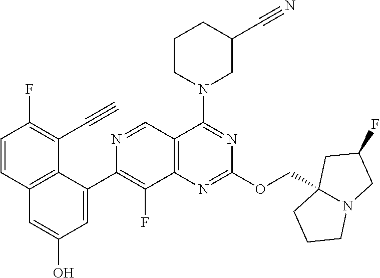 [SMILES: C#Cc1c(F)ccc2cc(O)cc(-c3ncc4c(N5CCCC(C#N)C5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4- yl)piperidine-3- carbonitrile | 599.2 | ¹H NMR (400 MHz, DMSO-d₆) δ ppm = 10.21- 10.15 (m, 1H), 9.13-9.06 (m, 1H), 8.03-7.95 (m, 1H), 7.47 (t, J = 9.0 Hz, 1H), 7.41 (d, J = 1.6 Hz, 1H), 7.22 (dd, J = 6.6, 2.5 Hz, 1H), 5.54-5.26 (m, 1H), 4.45-3.93 (m, 6H), 3.87-3.71 (m, 2H), 3.24 (br s, 4H), 2.28-1.77 (m, 10H) |
| 1-24 | 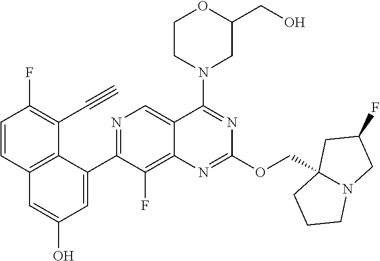 [SMILES: C#Cc1c(F)ccc2cc(O)cc(-c3ncc4c(N5CCOC(CO)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[2- (hydroxymethyl) morpholin-4- yl]pyrido[4,3- d]pyrimidin-7-yl)- 5-ethynyl-6- fluoronaphthalen-2- ol | 606.5 | |
| 1-25^{A} | 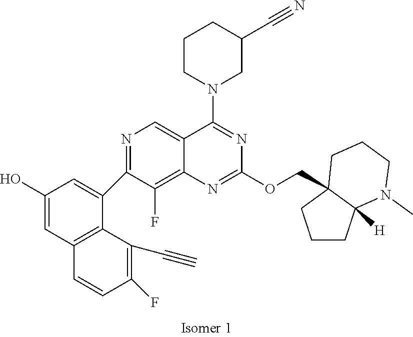 [SMILES: C#Cc1c(F)ccc2cc(O)cc(-c3ncc4c(N5CCCC(C#N)C5)nc(OC[C@]56CCC[C@H]5N(C)CCC6)nc4c3F)c12] | 1-(2-{[(4aS,7aR)- 1-methyl- octahydro-1H- cyclopenta[b] pyridin-4a- yl]methoxy}- 7-(8-ethynyl-7- fluoro-3- hydroxynaphthalen- 1-yl)-8- fluoropyrido[4,3- d]pyrimidin-4- yl)piperidine-3- carbonitrile | 609.3 | ¹H NMR (400 MHz, DMSO-d₆) δ ppm = 10.20 (br d, J = 1.3 Hz, 1H), 9.08 (d, J = 6.4 Hz, 1H), 7.99 (dd, J = 8.3, 6.6 Hz, 1H), 7.47 (t, J = 9.0 Hz, 1H), 7.41 (d, J = 2.3 Hz, 1H), 7.22 (dd, J = 4.3, 2.4 Hz, 1H), 4.39- 4.13 (m, 2H), 4.00-3.88 (m, 3H), 3.84-3.69 (m, 2H), 2.55-2.53 (m, 1H), 2.40-2.31 (m, 3H), 2.09- 2.01 (m, 2H), 1.99-1.88 (m, 3H), 1.81-1.72 (m, 2H), 1.64 (br d, J = 4.3 Hz, 6H), 1.56-1.45 (m, 3H), 1.18-1.12 (m, 1H) |
| 1-26^{A} | 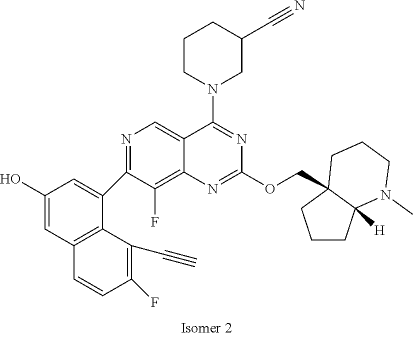 [SMILES: C#Cc1c(F)ccc2cc(O)cc(-c3ncc4c(N5CCCC(C#N)C5)nc(OC[C@]56CCC[C@H]5N(C)CCC6)nc4c3F)c12] | 1-(2-{[(4aS,7aR)-1- methyl-octahydro- 1H- cyclopenta[b] pyridin-4a- yl]methoxy}- 7-(8-ethynyl-7- fluoro-3- hydroxynaphthalen- 1-yl)-8- fluoropyrido[4,3- d]pyrimidin-4- yl)piperidine-3- carbonitrile | 609.3 | |
| 1-27 | 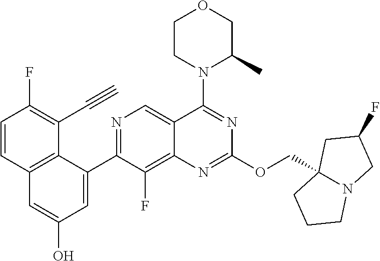 [SMILES: C#Cc1c(F)ccc2cc(O)cc(-c3ncc4c(N5CCOC[C@H]5C)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(3R)-3- methylmorpholin-4- yl]pyrido[4,3- d]pyrimidin-7-yl)- 5-ethynyl-6- fluoronaphthalen-2- ol | 590.3 | |
| 1-28 | 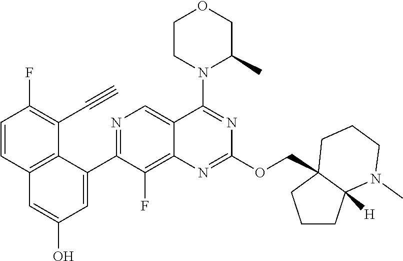 [SMILES: C#Cc1c(F)ccc2cc(O)cc(-c3ncc4c(N5CCOC[C@H]5C)nc(OC[C@]56CCC[C@H]5N(C)CCC6)nc4c3F)c12] | 4-(2-{[(4aS,7aR)-1- methyl-octahydro- 1H- cyclopenta[b] pyridin-4a- yl]methoxy}- 8-fluoro-4-[(3R)-3- methylmorpholin-4- yl]pyrido[4,3- d]pyrimidin-7-yl)- 5-ethynyl-6- fluoronaphthalen-2- ol | 600.3 | |
| 1-29 | 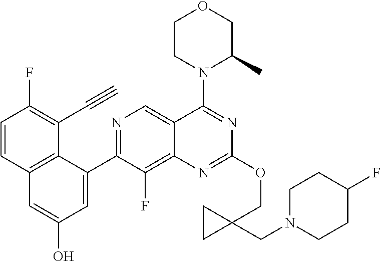 [SMILES: C#Cc1c(F)ccc2cc(O)cc(-c3ncc4c(N5CCOC[C@H]5C)nc(OCC5(CN6CCC(F)CC6)CC5)nc4c3F)c12] | 5-ethynyl-6-fluoro- 4-[8-fluoro-2-({1- [(4-fluoropiperidin- 1-yl)methyl] cyclopropyl} methoxy)-4- [(3R)-3- methylmorpholin- 4-yl]pyrido[4,3- d]pyrimidin-7- yl]naphthalen- 2-ol | 618.3 | |
| 1-30 | 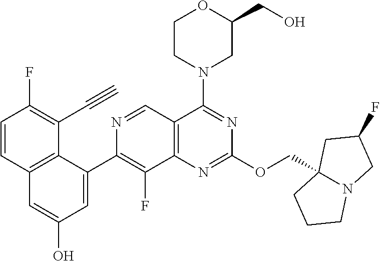 [SMILES: C#Cc1c(F)ccc2cc(O)cc(-c3ncc4c(N5CCO[C@@H](CO)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(2R)-2- (hydroxymethyl) morpholin-4- yl]pyrido[4,3- d]pyrimidin-7-yl)- 5-ethynyl-6- fluoronaphthalen- 2-ol | 606.3 | |
| 1-31 | 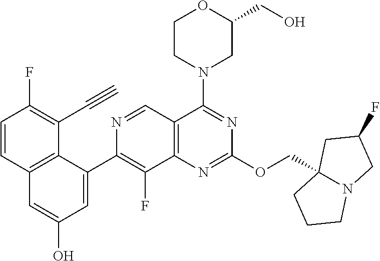 [SMILES: C#Cc1c(F)ccc2cc(O)cc(-c3ncc4c(N5CCO[C@H](CO)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(2S)-2- (hydroxymethyl) morpholin-4- yl]pyrido[4,3- d]pyrimidin-7-yl)- 5-ethynyl-6- fluoronaphthalen- 2-ol | 606.3 | |
| 1-32 | 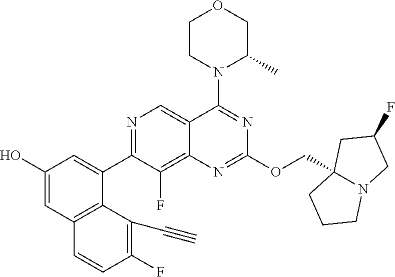 [SMILES: C#Cc1c(F)ccc2cc(O)cc(-c3ncc4c(N5CCOC[C@@H]5C)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(3S)-3- methylmorpholin- 4-yl]pyrido[4,3- d]pyrimidin-7-yl)- 5-ethynyl-6- fluoronaphthalen- 2-ol | 590.3 | |
| 1-33 | 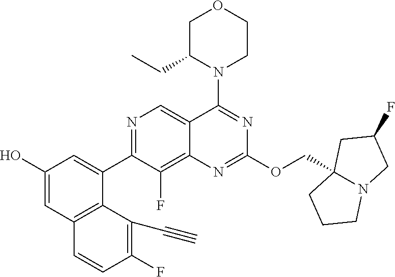 [SMILES: C#Cc1c(F)ccc2cc(O)cc(-c3ncc4c(N5CCOC[C@H]5CC)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4- [(3R)-3- ethylmorpholin- 4-yl]-8- fluoropyrido[4,3- d]pyrimidin-7-yl)- 5-ethynyl-6- fluoronaphthalen- 2-ol | 604.3 | |
| 1-34 | 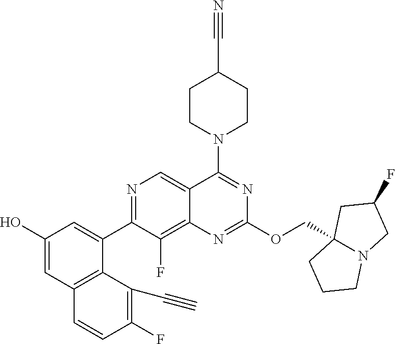 [SMILES: C#Cc1c(F)ccc2cc(O)cc(-c3ncc4c(N5CCC(C#N)CC5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4- yl)piperidine-4- carbonitrile | 599.3 | ¹H NMR (400 MHz, DMSO-d₆) δ ppm = 10.20 (br d, J = 1.3 Hz, 1H), 9.08 (d, J = 6.4 Hz, 1H), 7.99 (dd, J = 8.3, 6.6 Hz, 1H), 7.47 (t, J = 9.0 Hz, 1H), 7.41 (d, J = 2.3 Hz, 1H), 7.22 (dd, J = 4.3, 2.4 Hz, 1H), 4.39-4.13 (m, 2H), 4.00-3.88 (m, 3H), 3.84- 3.69 (m, 2H), 2.55-2.53 (m, 1H), 2.40-2.31 (m, 3H), 2.09-2.01 (m, 2H), 1.99-1.88 (m, 3H), 1.81- 1.72 (m, 2H), 1.64 (br d, J = 4.3 Hz, 6H), 1.56- 1.45 (m, 3H), 1.18-1.12 (m, 1H) |
| 1-35 | 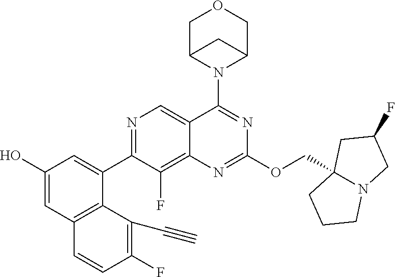 [SMILES: C#Cc1c(F)ccc2cc(O)cc(-c3ncc4c(N5C6COCC5C6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{3-oxa-6- azabicyclo[3.1.1] heptan-6- yl } pyrido[4,3- d]pyrimidin-7-yl)- 5-ethynyl-6- fluoronaphthalen- 2-ol | 588.3 | ¹H NMR (400 MHz, DMSO-d₆) δ ppm = 10.17 (s, 1H), 9.01 (s, 1H), 7.98 (dd, J = 9.2, 5.9 Hz, 1H), 7.52-7.44 (m, 1H), 7.40 (d, J = 2.6 Hz, 1H), 7.21-7.15 (m, 1H), 5.50-5.24 (m, 1H), 4.94-4.75 (m, 1H), 4.39-4.06 (m, 4H), 4.04- 3.93 (m, 3H), 3.49 (s, 1H), 3.18 (br d, J = 4.9 Hz, 2H), 3.02-2.90 (m, 2H), 2.29- 2.13 (m, 2H), 2.10-2.01 (m, 2H), 2.01-1.80 (m, 4H) |
| 1-36 | 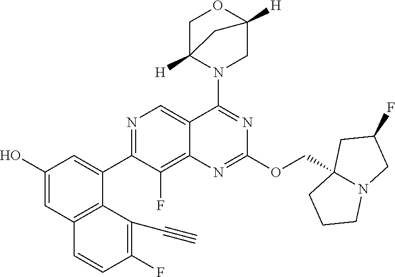 [SMILES: C#Cc1c(F)ccc2cc(O)cc(-c3ncc4c(N5C[C@@H]6C[C@H]5CO6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(1S,4S)- 2-oxa-5- azabicyclo[2.2.1] heptan-5- yl]pyrido[4,3- d]pyrimidin-7-yl)- 5-ethynyl-6- fluoronaphthalen- 2-ol | 588.2 | |
| 1-37 | 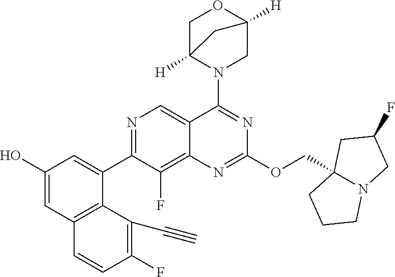 [SMILES: C#Cc1c(F)ccc2cc(O)cc(-c3ncc4c(N5C[C@H]6C[C@@H]5CO6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(1R,4R)- 2-oxa-5- azabicyclo[2.2.1] heptan-5- yl]pyrido[4,3- d]pyrimidin-7-yl)- 5-ethynyl-6- fluoronaphthalen- 2-ol | 588.2 | ¹H NMR (400 MHz, DMSO-d₆) δ ppm = 10.16 (br s, 1H), 9.14 (br d, J = 10.3 Hz, 1H), 7.98 (br dd, J = 9.0, 6.4 Hz, 1H), 7.51-7.44 (m, 1H), 7.40 (br d, J = 2.3 Hz, 1H), 7.16 (br s, 1H), 5.52-5.22 (m, 2H), 4.85-4.79 (m, 1H), 4.32-4.09 (m, 3H), 4.06- 3.83 (m, 4H), 3.02-2.82 (m, 2H), 2.39-2.31 (m, 4H), 2.21-1.71 (m, 6H) |
| 1-38 | 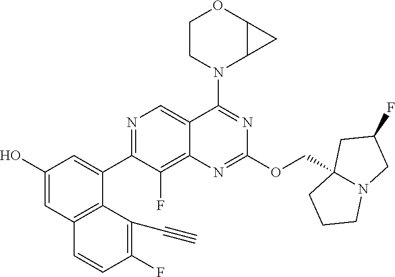 [SMILES: C#Cc1c(F)ccc2cc(O)cc(-c3ncc4c(N5CCOC6CC65)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{2-oxa-5- azabicyclo[4.1.0] heptan-5- yl }pyrido[4,3- d]pyrimidin-7-yl)- 5-ethynyl-6- fluoronaphthalen- 2-ol | 588.2 | ¹H NMR (400 MHz, DMSO-d₆) δ ppm = 10.17 (s, 1H), 9.73 (d, J = 16.4 Hz, 1H), 7.98 (dd, J = 9.0, 6.0 Hz, 1H), 7.47 (td, J = 9.1, 1.4 Hz, 1H), 7.40 (d, J = 2.4 Hz, 1H), 7.23-7.17 (m, 1H), 5.49-5.26 (m, 1H), 4.34-4.14 (m, 3H), 4.10-3.96 (m, 3H), 3.95- 3.87 (m, 2H), 3.59-3.46 (m, 3H), 3.03-2.87 (m, 1H), 2.29-2.05 (m, 3H), 2.01-1.80 (m, 3H), 1.34- 1.06 (m, 3H) |
| 1-39 | 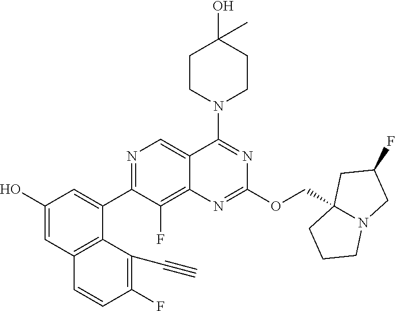 [SMILES: C#Cc1c(F)ccc2cc(O)cc(-c3ncc4c(N5CCC(C)(O)CC5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 4-methylpiperidin- 4-ol | 604.3 | |
| 1-40 | 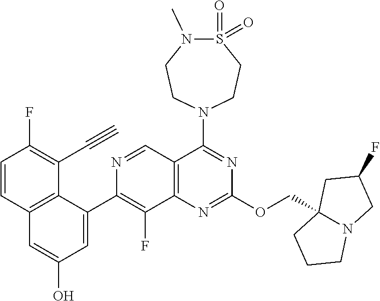 [SMILES: C#Cc1c(F)ccc2cc(O)cc(-c3ncc4c(N5CCN(C)S(=O)(=O)CC5)nc(OC[C@@]56CCCN5C[C@H](F)C6)nc4c3F)c12] | 5-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 2-methyl-1¿6,2,5- thiadiazepane-1,1- dione | 653.2 | |
| 1-41 | 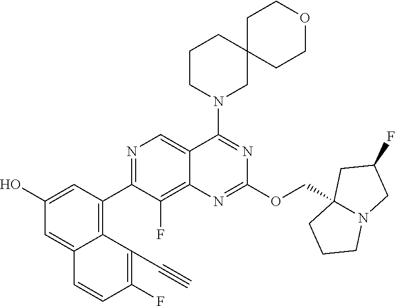 [SMILES: C#Cc1c(F)ccc2cc(O)cc(-c3ncc4c(N5CCCC6(CCOCC6)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{9-oxa-2- azaspiro[5.5] undecan-2-yl} pyrido[4,3- d]pyrimidin-7-yl)- 5-ethynyl-6- fluoronaphthalen- 2-ol | 644.3 | |
| 1-42^{B} | 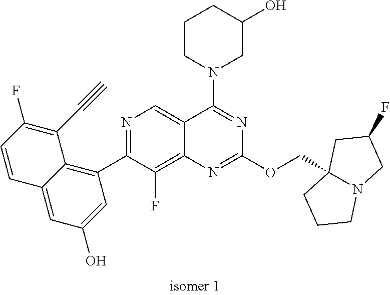 [SMILES: C#Cc1c(F)ccc2cc(O)cc(-c3ncc4c(N5CCCC(O)C5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4- yl)piperidin-3-ol | 590.2 | |
| 1-43^{B} | 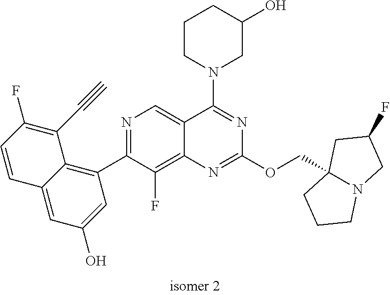 [SMILES: C#Cc1c(F)ccc2cc(O)cc(-c3ncc4c(N5CCCC(O)C5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4- yl)piperidin-3-ol | 590.3 | |
| 1-44^{C} | 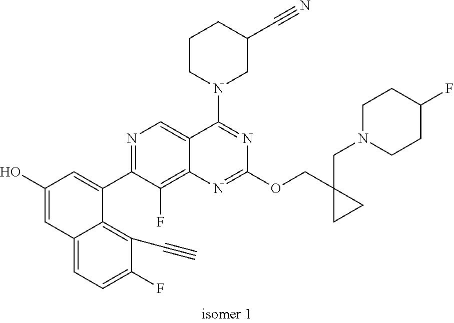 [SMILES: C#Cc1c(F)ccc2cc(O)cc(-c3ncc4c(N5CCCC(C#N)C5)nc(OCC5(CN6CCC(F)CC6)CC5)nc4c3F)c12] | 1-[7-(8-ethynyl-7- fluoro-3- hydroxynaphthalen- 1-yl)-8-fluoro-2- ({1-[(4- fluoropiperidin-1- yl)methyl] cyclopropyl} methoxy)pyrido [4,3-d]pyrimidin- 4-yl]piperidine-3- carbonitrile | 627.3 | |
| 1-45^{C} | 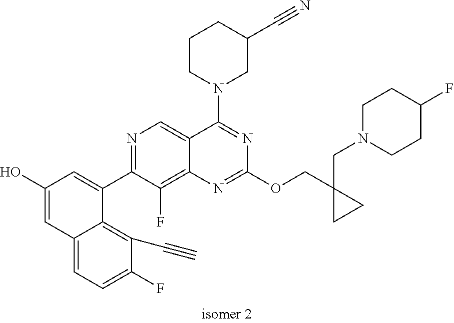 [SMILES: C#Cc1c(F)ccc2cc(O)cc(-c3ncc4c(N5CCCC(C#N)C5)nc(OCC5(CN6CCC(F)CC6)CC5)nc4c3F)c12] | 1-[7-(8-ethynyl-7- fluoro-3- hydroxynaphthalen- 1-yl)-8-fluoro-2- ({1-[(4- fluoropiperidin-1- yl)methyl] cyclopropyl} methoxy)pyrido [4,3-d]pyrimidin- 4-yl]piperidine-3- carbonitrile | 627.3 | |
| 1-46 | 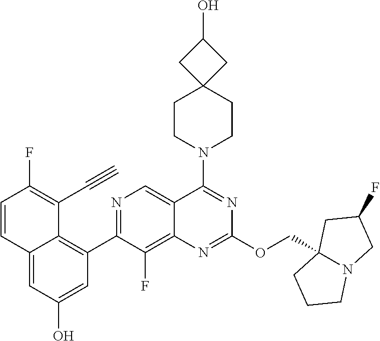 [SMILES: C#Cc1c(F)ccc2cc(O)cc(-c3ncc4c(N5CCC6(CC5)CC(O)C6)nc(OC[C@@]56CCCN5C[C@H](F)C6)nc4c3F)c12] | 7-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 7-azaspiro[3.5] nonan-2-ol | 630.3 | |
| 1-47 | 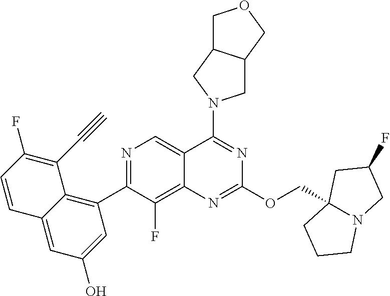 [SMILES: C#Cc1c(F)ccc2cc(O)cc(-c3ncc4c(N5CC6COCC6C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4- {hexahydro-1H- furo[3,4-c]pyrrol- 5-yl}pyrido[4,3- d]pyrimidin-7-yl)- 5-ethynyl-6- fluoronaphthalen- 2-ol | 602.3 | |
| 1-48 | 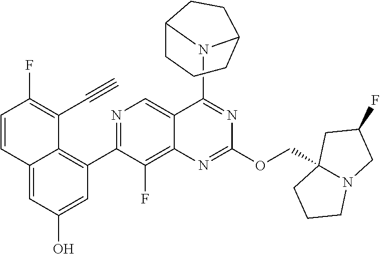 [SMILES: C#Cc1c(F)ccc2cc(O)cc(-c3ncc4c(N5C6CCCC5CC6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-{8- azabicyclo[3.2.1] octan-8-yl}-8- fluoropyrido[4,3- d]pyrimidin-7-yl)- 5-ethynyl-6- fluoronaphthalen- 2-ol | 600.3 | |
| 1-49 | 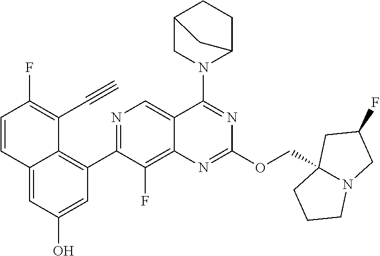 [SMILES: C#Cc1c(F)ccc2cc(O)cc(-c3ncc4c(N5CC6CCC5C6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-{2- azabicyclo[2.2.1 ] heptan-2-yl}-8- fluoropyrido[4,3- d]pyrimidin-7-yl)- 5-ethynyl-6- fluoronaphthalen- 2-ol | 586.2 | |
| 1-50 | 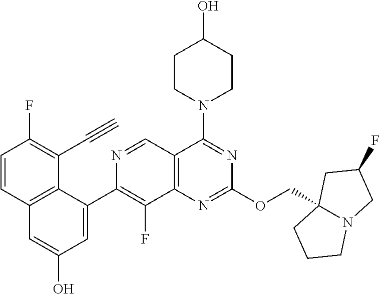 [SMILES: C#Cc1c(F)ccc2cc(O)cc(-c3ncc4c(N5CCC(O)CC5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4- yl)piperidin-4-ol | 590.3 | |
| 1-51 | 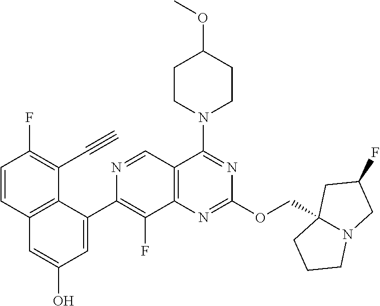 [SMILES: C#Cc1c(F)ccc2cc(O)cc(-c3ncc4c(N5CCC(OC)CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-(4- methoxypiperidin- 1-yl)pyrido[4,3- d]pyrimidin-7-yl)- 5-ethynyl-6- fluoronaphthalen- 2-ol | 604.3 | |
| 1-52 | 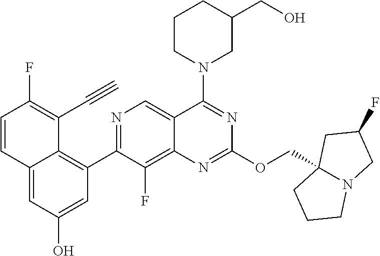 [SMILES: C#Cc1c(F)ccc2cc(O)cc(-c3ncc4c(N5CCCC(CO)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[3- (hydroxymethyl) piperidin-1- yl]pyrido[4,3- d]pyrimidin-7-yl)- 5-ethynyl-6- fluoronaphthalen- 2-ol | 604.3 | ¹H NMR (400 MHz, DMSO-d₆) δ ppm = 9.01 (s, 1H), 7.92 (dd, J = 9.3, 6.0 Hz, 1H), 7.42 (t, J = 9.1 Hz, 1H), 7.34 (d, J = 2.5 Hz, 1H), 7.18 (d, J = 2.5 Hz, 1H), 5.40-5.18 (m, 1H), 4.61-4.36 (m, 2H), 4.18- 4.05 (m, 2H), 3.96-3.93 (m, 2H), 3.57-3.52 (m, 3H), 3.35-3.22 (m, 4H), 3.14- 3.10 (m, 3H), 2.84-2.80 (m, 1H), 2.14-2.11 (m, 1H), 2.06 -2.03 (m, 2H), 1.83- 1.77 (m, 5H). |
| 1-53 | 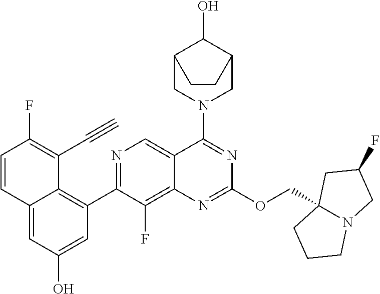 [SMILES: C#Cc1c(F)ccc2cc(O)cc(-c3ncc4c(N5CC6CCC(C5)C6O)nc(OC[C@@]56CCCN5C[C@H](F)C6)nc4c3F)c12] | 3-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 3-azabicyclo [3.2.1]octan-8-ol | 604.3 | |
| 1-54 | 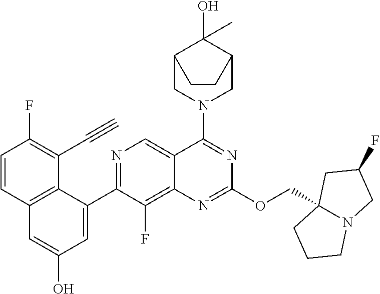 [SMILES: C#Cc1c(F)ccc2cc(O)cc(-c3ncc4c(N5CC6CCC(C5)C6(C)O)nc(OC[C@@]56CCCN5C[C@H](F)C6)nc4c3F)c12] | 3-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 8-methyl-3- azabicyclo[3.2.1] octan-8-ol | 630.3 | |
| 1-55 | 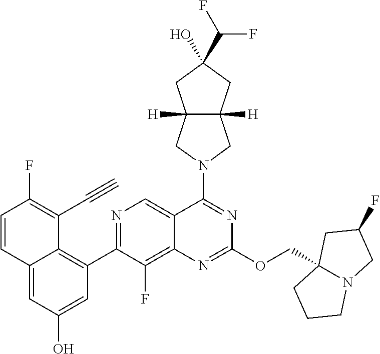 [SMILES: C#Cc1c(F)ccc2cc(O)cc(-c3ncc4c(N5C[C@@H]6C[C@@](O)(C(F)F)C[C@@H]6C5)nc(OC[C@@]56CCCN5C[C@H](F)C6)nc4c3F)c12] | (3aR,5R,6aS)-2-(2- {[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 5-(difluoromethyl)- octahydrocyclopent a[c]pyrrol-5-ol | 666.3 | |
| 1-56 | 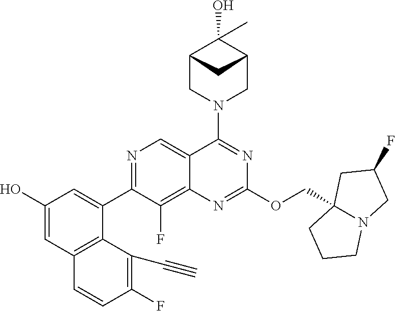 [SMILES: C#Cc1c(F)ccc2cc(O)cc(-c3ncc4c(N5C[C@H]6C[C@@H](C5)[C@@]6(C)O)nc(OC[C@@]56CCCN5C[C@H](F)C6)nc4c3F)c12] | (1R,5S,6R)-3-(2- {[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 6-methyl-3- azabicyclo[3.1.1] heptan-6-ol | 616.3 | |
| 1-57 | 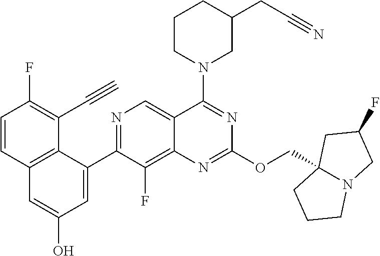 [SMILES: C#Cc1c(F)ccc2cc(O)cc(-c3ncc4c(N5CCCC(CC#N)C5)nc(OC[C@@]56CCCN5C[C@H](F)C6)nc4c3F)c12] | 2-[1-(2-{[(2R,7aS)- 2-fluoro- hexahydro-1H- pyrrolizin-7a- yl]methoxy}-7-(8- ethynyl-7-fluoro-3- hydroxynaphthalen- 1-yl)-8- fluoropyrido[4,3- d]pyrimidin-4- yl)piperidin-3- yl]acetonitrile | 613.3 | |
| 1-58 | 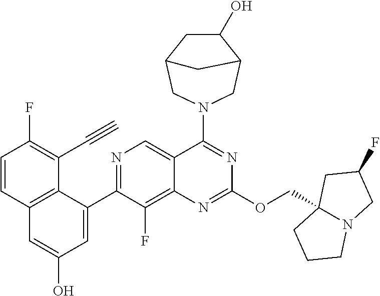 [SMILES: C#Cc1c(F)ccc2cc(O)cc(-c3ncc4c(N5CC6CC(O)C(C6)C5)nc(OC[C@@]56CCCN5C[C@H](F)C6)nc4c3F)c12] | 3-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 3-azabicyclo [3.2.1]octan-6-ol | 616.2 | |
| 1-59 | 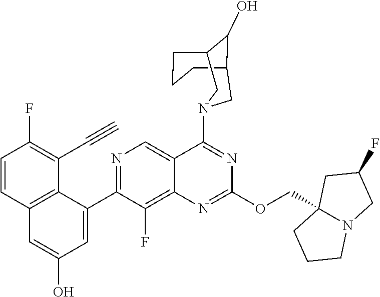 [SMILES: C#Cc1c(F)ccc2cc(O)cc(-c3ncc4c(N5CC6CCCC(C5)C6O)nc(OC[C@@]56CCCN5C[C@H](F)C6)nc4c3F)c12] | 3-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 3-azabicyclo [3.3.1]nonan-9-ol | 630.3 | |
| 1-60^{D} | 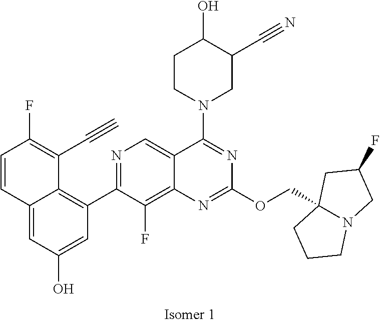 [SMILES: C#Cc1c(F)ccc2cc(O)cc(-c3ncc4c(N5CCC(O)C(C#N)C5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 4- hydroxypiperidine- 3-carbonitrile | 615.2 | |
| 1-61^{D} | 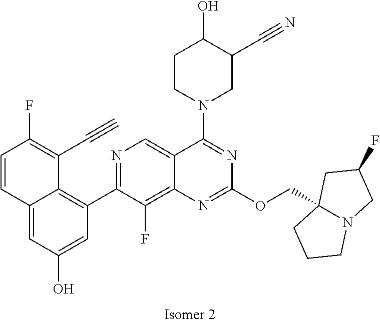 [SMILES: C#Cc1c(F)ccc2cc(O)cc(-c3ncc4c(N5CCC(O)C(C#N)C5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 4- hydroxypiperidine- 3-carbonitrile | 615.2 | |
| 1-62^{E} | 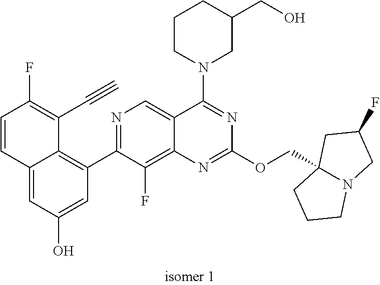 [SMILES: C#Cc1c(F)ccc2cc(O)cc(-c3ncc4c(N5CCCC(CO)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[3- (hydroxymethyl) piperidin-1- yl]pyrido[4,3- d]pyrimidin-7-yl)- 5-ethynyl-6- fluoronaphthalen- 2-ol | 604.3 | |
| 1-63^{E} | 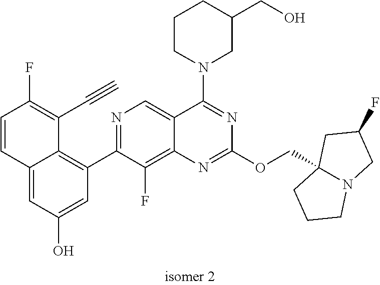 [SMILES: C#Cc1c(F)ccc2cc(O)cc(-c3ncc4c(N5CCCC(CO)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[3- (hydroxymethyl) piperidin-1- yl]pyrido[4,3- d]pyrimidin-7-yl)- 5-ethynyl-6- fluoronaphthalen- 2-ol | 604.2 | |
| 1-64 | 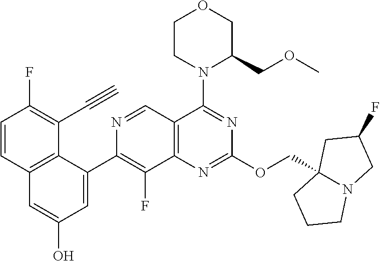 [SMILES: C#Cc1c(F)ccc2cc(O)cc(-c3ncc4c(N5CCOC[C@H]5COC)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(3R)-3- (methoxymethyl) morpholin-4- yl]pyrido[4,3- d]pyrimidin-7-yl)- 5-ethynyl-6- fluoronaphthalen- 2-ol | 620.2 | |
| 1-65 | 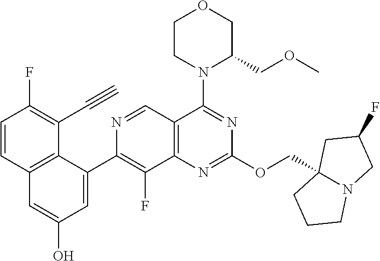 [SMILES: C#Cc1c(F)ccc2cc(O)cc(-c3ncc4c(N5CCOC[C@@H]5COC)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(3S)-3- (methoxymethyl) morpholin-4- yl]pyrido[4,3- d]pyrimidin-7-yl)- 5-ethynyl-6- fluoronaphthalen- 2-ol | 620.2 | |
| 1-66 | 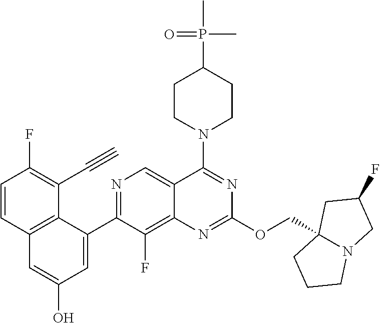 [SMILES: C#Cc1c(F)ccc2cc(O)cc(-c3ncc4c(N5CCC(P(C)(C)=O)CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-[4- (dimethylphosphory 1)piperidin-1-yl]-8- fluoropyrido[4,3- d]pyrimidin-7-yl)- 5-ethynyl-6- fluoronaphthalen- 2-ol | 650.2 | |
| 1-67 | 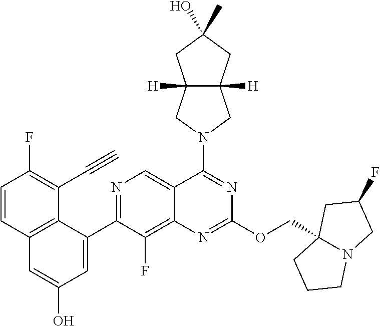 [SMILES: C#Cc1c(F)ccc2cc(O)cc(-c3ncc4c(N5C[C@@H]6C[C@](C)(O)C[C@@H]6C5)nc(OC[C@@]56CCCN5C[C@H](F)C6)nc4c3F)c12] | (3aR,5S,6aS)-2-(2- {[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 5-methyl- octahydrocyclopenta [c]pyrrol-5-ol | 630.3 | |
| 1-68 | 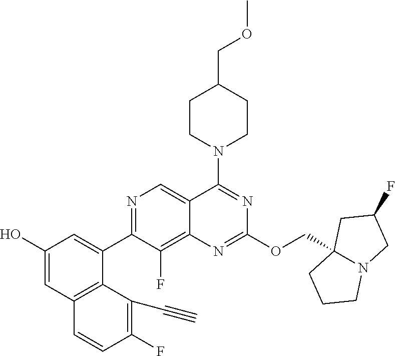 [SMILES: C#Cc1c(F)ccc2cc(O)cc(-c3ncc4c(N5CCC(COC)CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[4- (methoxymethyl) piperidin-1- yl]pyrido[4,3- d]pyrimidin-7-yl)- 5-ethynyl-6- fluoronaphthalen- 2-ol | 618.2 | |
| 1-69 | 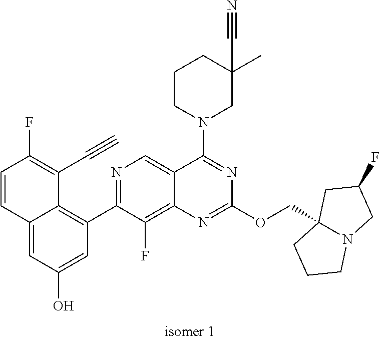 [SMILES: C#Cc1c(F)ccc2cc(O)cc(-c3ncc4c(N5CCCC(C)(C#N)C5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 3-methylpiperidine- 3-carbonitrile | 613.2 | |
| 1-70 | 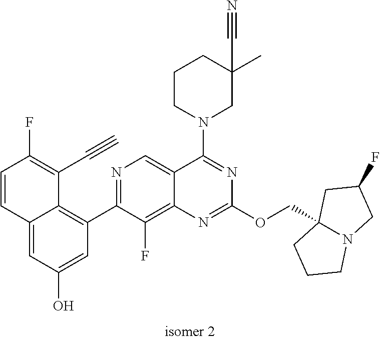 [SMILES: C#Cc1c(F)ccc2cc(O)cc(-c3ncc4c(N5CCCC(C)(C#N)C5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 3-methylpiperidine- 3-carbonitrile | 613.2 | |
| 1-71^{F} | 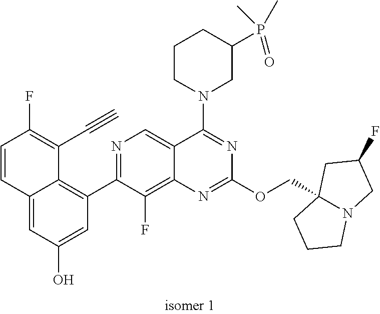 [SMILES: C#Cc1c(F)ccc2cc(O)cc(-c3ncc4c(N5CCCC(P(C)(C)=O)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-[3- (dimethylphosphory 1)piperidin-1-yl]-8- fluoropyrido[4,3- d]pyrimidin-7-yl)- 5-ethynyl-6- fluoronaphthalen- 2-ol | 650.3 | |
| 1-72^{F} | 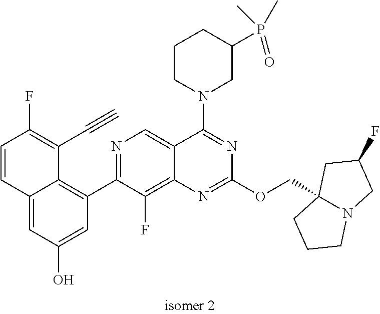 [SMILES: C#Cc1c(F)ccc2cc(O)cc(-c3ncc4c(N5CCCC(P(C)(C)=O)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-[3- (dimethylphosphory 1)piperidin-1-yl]-8- fluoropyrido[4,3- d]pyrimidin-7-yl)- 5-ethynyl-6- fluoronaphthalen- 2-ol | 650.3 | |
| 1-73^{G} | 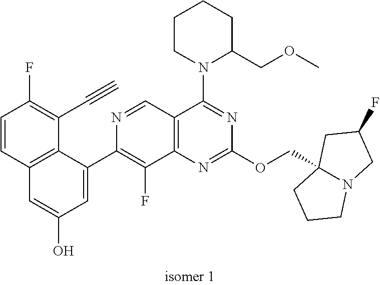 [SMILES: C#Cc1c(F)ccc2cc(O)cc(-c3ncc4c(N5CCCCC5COC)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[2- (methoxymethyl) piperidin-1- yl]pyrido[4,3- d]pyrimidin-7-yl)- 5-ethynyl-6- fluoronaphthalen- 2-ol | 618.2 | |
| 1-74^{G} | 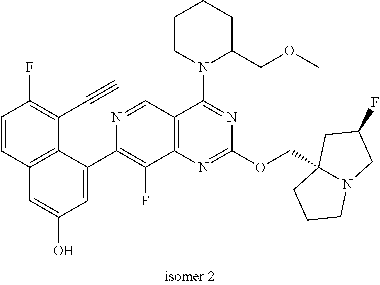 [SMILES: C#Cc1c(F)ccc2cc(O)cc(-c3ncc4c(N5CCCCC5COC)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[2- (methoxymethyl) piperidin-1- yl]pyrido[4,3- d]pyrimidin-7-yl)- 5-ethynyl-6- fluoronaphthalen- 2-ol | 618.2 | |
| 1-75 | 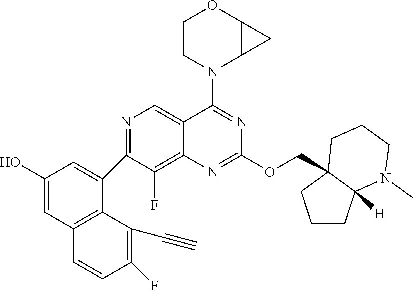 [SMILES: C#Cc1c(F)ccc2cc(O)cc(-c3ncc4c(N5CCOC6CC65)nc(OC[C@]56CCC[C@H]5N(C)CCC6)nc4c3F)c12] | 4-(2-{[(4aS,7aR)-1- methyl-octahydro- 1H- cyclopenta[b] pyridin-4a- yl]methoxy}- 8-fluoro-4- {2-oxa-5- azabicyclo[4.1.0] heptan-5- yl } pyrido[4,3- d]pyrimidin-7-yl)- 5-ethynyl-6- fluoronaphthalen- 2-ol | 598.2 | |
| 1-76 | 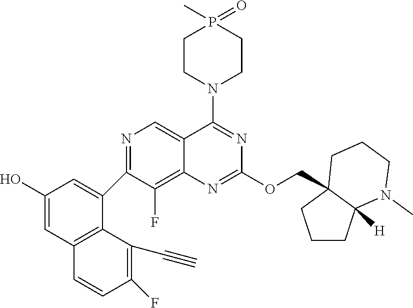 [SMILES: C#Cc1c(F)ccc2cc(O)cc(-c3ncc4c(N5CCP(C)(=O)CC5)nc(OC[C@]56CCC[C@H]5N(C)CCC6)nc4c3F)c12] | 1-(7-(8-ethynyl-7- fluoro-3- hydroxynaphthalen- 1-yl)-8-fluoro-2- (((4aS,7aR)-1- methyloctahydro- 4aH- cyclopenta[b] pyridin-4a- yl)methoxy)pyrido [4,3-d]pyrimidin- 4-yl)-4-methyl- 1,4-azaphosphinane 4-oxide | 632.2 | |
| 1-77 | 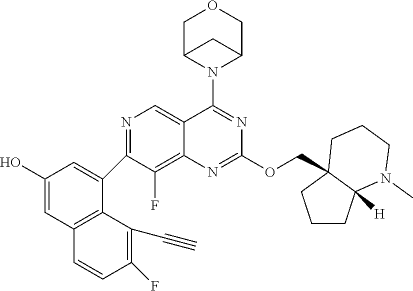 [SMILES: C#Cc1c(F)ccc2cc(O)cc(-c3ncc4c(N5C6COCC5C6)nc(OC[C@]56CCC[C@H]5N(C)CCC6)nc4c3F)c12] | 4-(2-{[(4aS,7aR)-1- methyl-octahydro- 1H- cyclopenta[b] pyridin-4a-yl] methoxy}- 8-fluoro-4- {3-oxa-6- azabicyclo[3.1.1] heptan-6- yl }pyrido[4,3- d]pyrimidin-7-yl)- 5-ethynyl-6- fluoronaphthalen- 2-ol | 598.2 | |
| 1-78 | 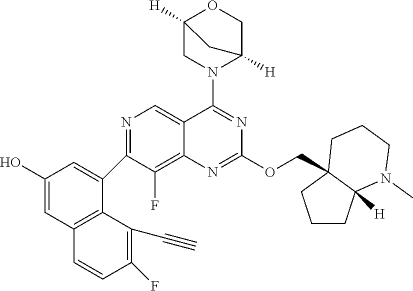 [SMILES: C#Cc1c(F)ccc2cc(O)cc(-c3ncc4c(N5C[C@@H]6C[C@H]5CO6)nc(OC[C@]56CCC[C@H]5N(C)CCC6)nc4c3F)c12] | 4-(2-{[(4aS,7aR)-1- methyl-octahydro- 1H- cyclopenta[b] pyridin-4a- yl]methoxy}- 8-fluoro-4- [(1S,4S)-2-oxa-5- azabicyclo[2.2.1] heptan-5- yl]pyrido[4,3- d]pyrimidin-7-yl)- 5-ethynyl-6- fluoronaphthalen- 2-ol | 598.3 | |
| 1-79 | 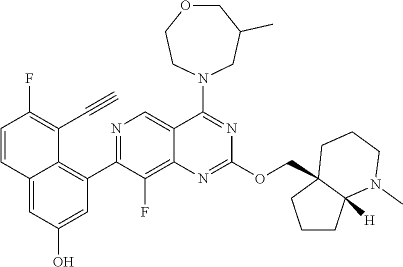 [SMILES: C#Cc1c(F)ccc2cc(O)cc(-c3ncc4c(N5CCOCC(C)C5)nc(OC[C@]56CCC[C@H]5N(C)CCC6)nc4c3F)c12] | 4-(2-{[(4aS,7aR)-1- methyl-octahydro- 1H-cyclopenta [b]pyridin-4a-yl] methoxy}-8- fluoro-4-(6- methyl-1,4- oxazepan-4- yl)pyrido[4,3- d]pyrimidin-7-yl)- 5-ethynyl-6- fluoronaphthalen- 2-ol | 614.3 | |
| 1-80 | 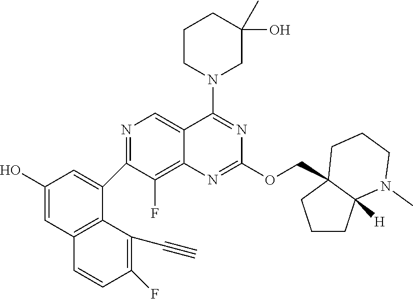 [SMILES: C#Cc1c(F)ccc2cc(O)cc(-c3ncc4c(N5CCCC(C)(O)C5)nc(OC[C@]56CCC[C@H]5N(C)CCC6)nc4c3F)c12] | 1-(2-{[(4aS,7aR)-1- methyl-octahydro- 1H-cyclopenta[b] pyridin-4a-yl] methoxy}-7-(8- ethynyl-7- fluoro-3- hydroxynaphthalen- 1-yl)-8- fluoropyrido[4,3- d]pyrimidin-4-yl)- 3-methylpiperidin- 3-ol | 614.2 | |
| 1-81 | 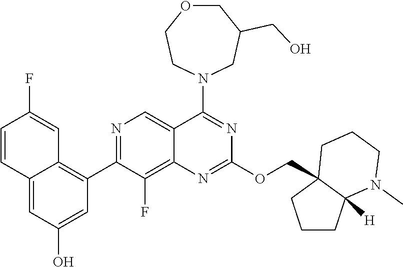 [SMILES: CN1CCC[C@@]2(COc3nc(N4CCOCC(CO)C4)c4cnc(-c5cc(O)cc6ccc(F)cc56)c(F)c4n3)CCC[C@@H]12] | 4-(2-{[(4aS,7aR)-1- methyl-octahydro- 1H-cyclopenta [b]pyridin-4a- yl]methoxy}- 8-fluoro-4-[6- (hydroxymethyl)- 1,4-oxazepan-4- yl]pyrido[4,3- d]pyrimidin-7-yl)- 5-ethynyl-6- fluoronaphthalen- 2-ol | 630.3 | |
| 1-82 | 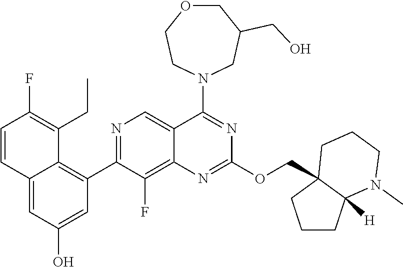 [SMILES: CCc1c(F)ccc2cc(O)cc(-c3ncc4c(N5CCOCC(CO)C5)nc(OC[C@]56CCC[C@H]5N(C)CCC6)nc4c3F)c12] | 4-(2-{[(4aS,7aR)-1- methyl-octahydro- 1H-cyclopenta [b]pyridin-4a- yl]methoxy}- 8-fluoro-4-[6- (hydroxymethyl)- 1,4-oxazepan-4- yl]pyrido[4,3- d]pyrimidin-7-yl)- 5-ethyl-6- fluoronaphthalen- 2-ol | 634.3 | |
| 1-83 | 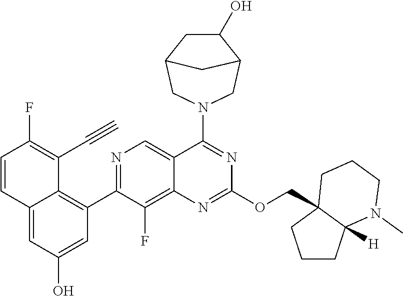 [SMILES: C#Cc1c(F)ccc2cc(O)cc(-c3ncc4c(N5CC6CC(O)C(C6)C5)nc(OC[C@]56CCC[C@H]5N(C)CCC6)nc4c3F)c12] | 3-(2-{[(4aS,7aR)-1- methyl-octahydro- 1H-cyclopenta [b]pyridi n-4a-yl]methoxy}- 7-(8-ethynyl-7- fluoro-3- hydroxynaphthalen- 1-yl)-8- fluoropyrido[4,3- d]pyrimidin-4-yl)- 3-azabicyclo [3.2.1]octan-6-ol | 626.2 | |
| 1-84 | 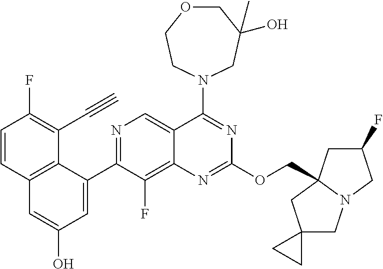 [SMILES: C#Cc1c(F)ccc2cc(O)cc(-c3ncc4c(N5CCOCC(C)(O)C5)nc(OC[C@@]56C[C@@H](F)CN5CC5(CC5)C6)nc4c3F)c12] | 4-(2-{[(6′R,7′aR)-6′- fluoro- hexahydrospiro [cyclopropane-1,2′- pyrrolizine]-7′a- yl]methoxy}-7-(8- ethynyl-7-fluoro-3- hydroxynaphthalen- 1-yl)-8- fluoropyrido[4,3- d]pyrimidin-4-yl)- 6-methyl-1,4- oxazepan-6-ol | 646.2 | |
| 1-85 | 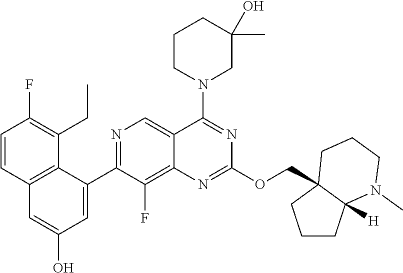 [SMILES: CCc1c(F)ccc2cc(O)cc(-c3ncc4c(N5CCCC(C)(O)C5)nc(OC[C@]56CCC[C@H]5N(C)CCC6)nc4c3F)c12] | 1-(2-{[(4aS,7aR)-1- methyl-octahydro- 1H-cyclopenta [b]pyridin-4a- yl]methoxy }- 7-(8-ethyl- 7-fluoro-3- hydroxynaphthalen- 1-yl)-8- fluoropyrido[4,3- d]pyrimidin-4-yl)- 3-methylpiperidin- 3-ol | 618.3 | ¹H NMR (400 MHz, DMSO-d₆) δ ppm = 9.22 (d, J = 2.1 Hz, 1H), 7.76 (dd, J = 9.0, 6.0 Hz, 1H), 7.40- 7.31 (m, 2H), 7.04 (d, J = 2.4 Hz, 1H), 4.83-4.70 (m, 1H), 4.51 (t, J = 10.9 Hz, 1H), 4.40-4.28 (m, 1H), 4.19-4.01 (m, 3H), 3.65- 3.59 (m, 1H), 2.65 (br d, J = 4.5 Hz, 1H), 2.24-2.12 (m, 7H), 1.77-1.48 (m, 14H), 1.18 (d, J = 8.4 Hz, 3H), 0.74 (q, J = 7.5 Hz, 3H). |
| 1-86 | 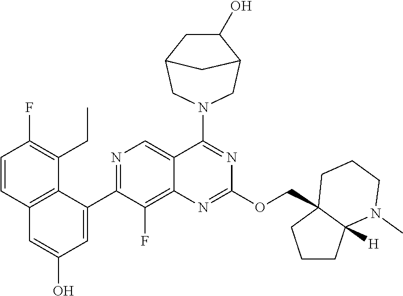 [SMILES: CCc1c(F)ccc2cc(O)cc(-c3ncc4c(N5CC6CC(O)C(C6)C5)nc(OC[C@]56CCC[C@H]5N(C)CCC6)nc4c3F)c12] | 3-(2-{[(4aS,7aR)-1- methyl-octahydro- 1H-cyclopenta[b] pyridin-4a- yl]methoxy}- 7-(8-ethyl-7- fluoro-3- hydroxynaphthalen- 1-yl)-8- fluoropyrido[4,3- d]pyrimidin- 4-yl)-3- azabicyclo[3.2.1] octan-6-ol | 630.3 | |
| 1-87 | 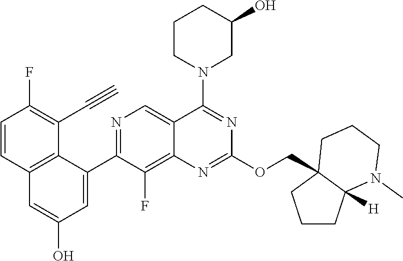 [SMILES: C#Cc1c(F)ccc2cc(O)cc(-c3ncc4c(N5CCC[C@@H](O)C5)nc(OC[C@]56CCC[C@H]5N(C)CCC6)nc4c3F)c12] | (3R)-1-(2- {[(4aS, 7aR)-1- methyl-octahydro- 1H-cyclopenta[b] pyridin-4a- yl]methoxy}- 7-(8-ethynyl-7- fluoro-3- hydroxynaphthalen- 1-yl)-8- fluoropyrido[4,3- d]pyrimidin-4- yl)piperidin-3-ol | 600.3 | ¹H NMR (400 MHz, DMSO-d₆) δ ppm = 10.18 (br s, 1H), 9.15-9.00 (m, 1H), 7.98 (dd, J = 9.1, 5.9 Hz, 1H), 7.47 (t, J = 9.1 Hz, 1H), 7.40 (d, J = 2.5 Hz, 1H), 7.21 (dd, J = 9.5, 2.5 Hz, 1H), 5.13 (t, J = 4.8 Hz, 1H), 4.48 (br d, J = 8.4 Hz, 1H), 4.24-4.13 (m, 2H), 3.99-3.70 (m, 4H), 3.63- 3.50 (m, 1H), 2.32-2.24 (m, 2H), 2.14-2.05 (m, 1H), 2.02-1.85 (m, 4H), 1.80-1.52 (m, 12H), 1.48- 1.41 (m, 1H). |
| 1-88 | 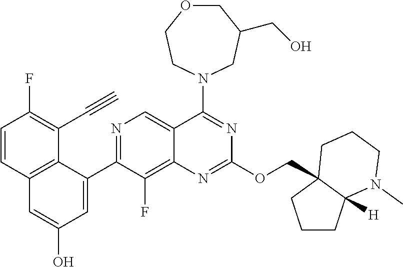 [SMILES: C#Cc1c(F)ccc2cc(O)cc(-c3ncc4c(N5CCOCC(CO)C5)nc(OC[C@]56CCC[C@H]5N(C)CCC6)nc4c3F)c12] | 4-(2-{[(4aS,7aR)-1- methyl-octahydro- 1H-cyclopenta[b] pyridin-4a- yl]methoxy}- 8-fluoro-4-[6- (hydroxymethyl)- 1,4-oxazepan-4- yl]pyrido[4,3- d]pyrimidin-7-yl)- 5-ethynyl-6- fluoronaphthalen- 2-ol | 630.2 | 1H NMR (400 MHz, DMSO-d₆) δ ppm = 10.16 (s, 1H), 9.16 (br d, J = 12.5 Hz, 1H), 7.98 (dd, J = 9.1, 5.9 Hz, 1H), 7.47 (t, J = 8.9 Hz, 1H), 7.40 (d, J = 2.1 Hz, 1H), 7.19 (dd, J = 13.3, 2.4 Hz, 1H), 4.86 (br d, J = 5.5 Hz, 1H), 4.58-4.39 (m, 2H), 4.35-4.13 (m, 2H), 4.08-3.83 (m, 4H), 3.80 - 3.65 (m, 1H), 3.56 (ddd, J = 14.7, 12.1, 7.8 Hz, 1H), 3.46 (br d, J = 5.5 Hz, 2H), 2.43-2.35 (m, 2H), 2.28- 2.14 (m, 2H), 2.00-1.52 (m, 9H), 1.48-1.40 (m, |
| | | | | 2H), 1.30-1.13 (m, 2H), |
| | | | | 0.91-0.76 (m, 1H). |
| 1-89 | 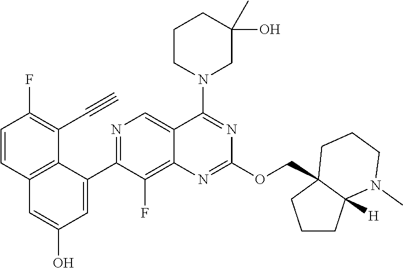 [SMILES: C#Cc1c(F)ccc2cc(O)cc(-c3ncc4c(N5CCCC(C)(O)C5)nc(OC[C@]56CCC[C@H]5N(C)CCC6)nc4c3F)c12] | 1-(2-{[(4aS,7aR)-1- methyl-octahydro- 1H-cyclopenta[b] pyridin-4a- yl]methoxy}- 7-(8-ethynyl-7- fluoro-3- hydroxynaphthalen- 1-yl)-8- fluoropyrido[4,3- d]pyrimidin-4-yl)- 3-methylpiperidin- 3-ol | 614.2 | 1H NMR (400 MHz, DMSO-d₆) δ = 10.37- 10.05 (m, 1H), 9.35-8.89 (m, 1H), 7.97 (dd, J = 9.2, 5.9 Hz, 1H), 7.63-7.09 (m, 3H), 4.85-4.72 (m, 1H), 4.54-4.43 (m, 1H), 4.20- 4.05 (m, 2H), 4.01-3.90 (m, 2H), 3.66-3.65 (m, 1H), 2.75-2.74 (m, 1H), 2.26-2.13 (m, 5H), 2.09- 1.95 (m, 1H), 1.92-1.78 (m, 1H), 1.76-1.47 (m, 11H), 1.44-1.34 (m, 1H), 1.17 (d, J = 15.8 Hz, 3H). |
| 1-90 | 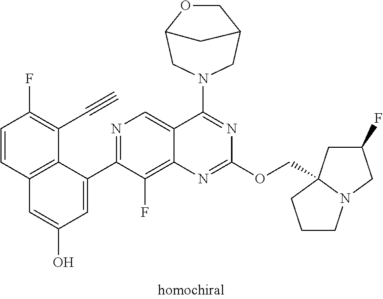 [SMILES: C#Cc1c(F)ccc2cc(O)cc(-c3ncc4c(N5CC6COC(C6)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{6-oxa- 3-azabicyclo [3.2.1]octan-3- yl}pyrido[4,3- d]pyrimidin-7-yl)- 5-ethynyl-6- fluoronaphthalen- 2-ol | 602.2 | ¹H NMR (400 MHz, DMSO-d₆) δ ppm = 9.13 (d, J = 8.9 Hz, 1H), 7.83 (br dd, J = 6.8, 5.4 Hz, 1H), 7.36 (br t, J = 8.8 Hz, 1H), 7.21 (br s, 1H), 7.10 (br s, 1H), 5.38-5.19 (m, 1H), 4.72-4.37 (m, 2H), 4.17- 4.08 (m, 1H), 4.06-3.98 (m, 1H), 3.92-3.68 (m, 4H), 3.07-3.07 (m, 1H), 2.88-2.80 (m, 2H), 2.72 (br d, J = 1.0 Hz, 1H), 2.17-2.11 (m, 1H), 2.09- 2.00 (m, 3H), 1.97-1.91 (m, 1H), 1.79 (br dd, J = 8.0, 4.0 Hz, 4H), 1.24 (s, 2H). |
| 1-91 | 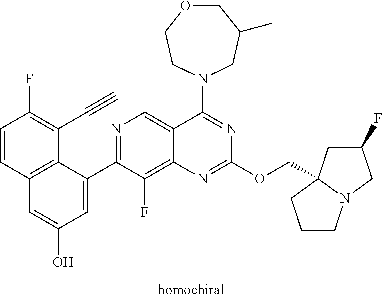 [SMILES: C#Cc1c(F)ccc2cc(O)cc(-c3ncc4c(N5CCOCC(C)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy }-8- fluoro-4-(6-methyl- 1,4-oxazepan-4- yl)pyrido[4,3- d]pyrimidin-7-yl)- 5-ethynyl-6- fluoronaphthalen-2- ol | 604.3 | |
| 1-92 | 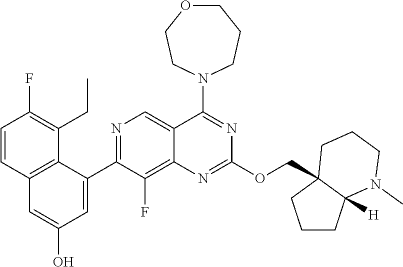 [SMILES: CCc1c(F)ccc2cc(O)cc(-c3ncc4c(N5CCCOCC5)nc(OC[C@]56CCC[C@H]5N(C)CCC6)nc4c3F)c12] | 4-(2-{[(4aS,7aR)-1- methyl-octahydro- 1H-cyclopenta [b]pyridin-4a- yl]methoxy}- 8-fluoro-4-(1,4- oxazepan-4- yl)pyrido[4,3- d]pyrimidin-7-yl)- 5-ethyl-6- fluoronaphthalen- 2-ol | 604.3 | |
| 1-93 | 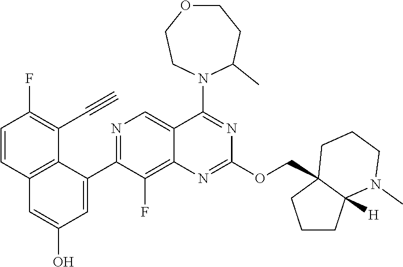 [SMILES: C#Cc1c(F)ccc2cc(O)cc(-c3ncc4c(N5CCOCCC5C)nc(OC[C@]56CCC[C@H]5N(C)CCC6)nc4c3F)c12] | 4-(2-{[(4aS,7aR)-1- methyl-octahydro- 1H-cyclopenta [b]pyridin-4a- yl]methoxy}- 8-fluoro-4-(5- methyl-1,4- oxazepan-4- yl)pyrido[4,3- d]pyrimidin-7-yl)- 5-ethynyl-6- fluoronaphthalen- 2-ol | 614.3 | |
| 1-94 | 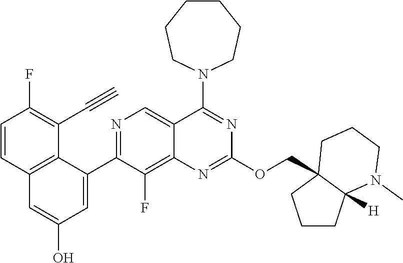 [SMILES: C#Cc1c(F)ccc2cc(O)cc(-c3ncc4c(N5CCCCCC5)nc(OC[C@]56CCC[C@H]5N(C)CCC6)nc4c3F)c12] | 4-(2-{[(4aS,7aR)-1- methyl-octahydro- 1H-cyclopenta [b]pyridin-4a- yl]methoxy}- 4-(azepan-1-yl)-8- fluoropyrido[4,3- d]pyrimidin-7-yl)- 5-ethynyl-6- fluoronaphthalen- 2-ol | 598.3 | |
| 1-95 | 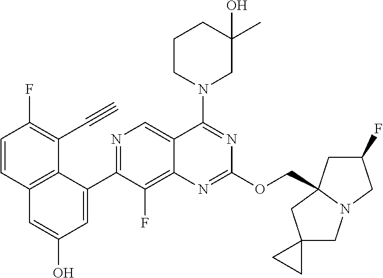 [SMILES: C#Cc1c(F)ccc2cc(O)cc(-c3ncc4c(N5CCCC(C)(O)C5)nc(OC[C@@]56C[C@@H](F)CN5CC5(CC5)C6)nc4c3F)c12] | 1-(2-{[(6'R,T'aR)- 6′-fluoro- hexahydrospiro [cyclopropane- 1,2′-pyrrolizine]- 7′a-yl]methoxy}- 7-(8-ethynyl- 7-fluoro-3- hydroxynaphthalen- 1-yl)-8- fluoropyrido[4,3- d]pyrimidin-4-yl)- 3-methylpiperidin- 3-ol | 630.3 | ¹H NMR (400 MHz, MeOD) δ ppm = 9.20-9.07 (m, 1H), 7.88-7.84 (m, 1H), 7.36-7.30 (m, 3H), 5.47-5.34 (m, 1H), 4.60- 4.48 (m, 1H), 4.49-3.48 (m, 2H), 4.38-3.34 (m, 1H), 3.67-3.39 (m, 4H), 3.07-3.03 (m, 2H), 2.72- 2.70 (m, 1H), 2.69-2.55 (m, 1H), 2.22-2.13 (m, 3H), 1.85-1.76 (m, 4H), 1.31-1.26 (m, 3H), 0.66- 0.57 (m, 4H). |
| 1-96 | 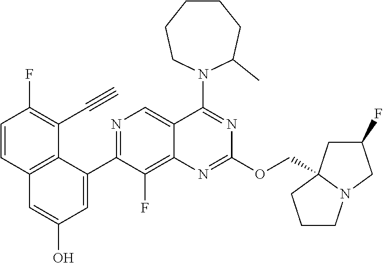 [SMILES: C#Cc1c(F)ccc2cc(O)cc(-c3ncc4c(N5CCCCCC5C)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-(5-methyl- 1,4-oxazepan-4- yl)pyrido[4,3- d]pyrimidin-7-yl)- 5-ethynyl-6- fluoronaphthalen- 2-ol | 604.2 | |
| 1-97 | 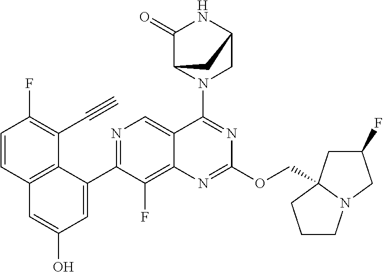 [SMILES: C#Cc1c(F)ccc2cc(O)cc(-c3ncc4c(N5C[C@@H]6C[C@H]5C(=O)N6)nc(OC[C@@]56CCCN5C[C@H](F)C6)nc4c3F)c12] | (1S,4S)-5-(2- {[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 2,5- diazabicyclo[2.2.1] heptan-3-one | 601.2 | |
| 1-98 | 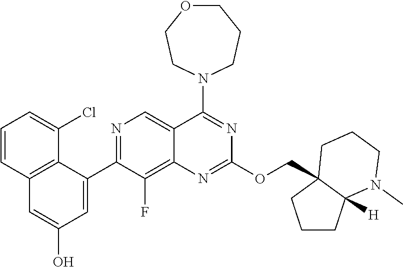 [SMILES: CN1CCC[C@@]2(COc3nc(N4CCCOCC4)c4cnc(-c5cc(O)cc6cccc(Cl)c56)c(F)c4n3)CCC[C@@H]12] | 4-(2-{[(4aS,7aR)-1- methyl-octahydro- 1H-cyclopenta[b] pyridin-4a- yl]methoxy}- 8-fluoro-4-(1,4- oxazepan-4- yl)pyrido[4,3- d]pyrimidin-7-yl)- 5-chloronaphthalen- 2-ol | 592.2 | |
| 1-99 | 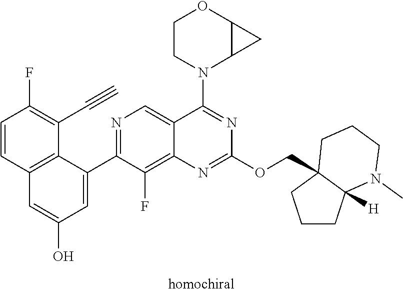 [SMILES: C#Cc1c(F)ccc2cc(O)cc(-c3ncc4c(N5CCOC6CC65)nc(OC[C@]56CCC[C@H]5N(C)CCC6)nc4c3F)c12] | 4-(2-{[(4aS,7aR)-1- methyl-octahydro- 1H-cyclopenta[b] pyridin-4a- yl]methoxy}- 8-fluoro-4- {2-oxa-5- azabicyclo[4.1.0] heptan-5- yl}pyrido[4,3- d]pyrimidin-7-yl)- 5-ethynyl-6- fluoronaphthalen- 2-ol | 598.3 | ¹H NMR (400 MHz, DMSO-d₆) δ = 10.25- 10.07 (m, 1H), 9.83-9.59 (m, 1H), 7.98 (dd, J = 9.2, 5.9 Hz, 1H), 7.52-7.42 (m, 1H), 7.41-7.37 (m, 1H), 7.20 (dd, J = 6.6, 2.6 Hz, 1H), 4.46 (d, J = 10.6 Hz, 1H), 4.24-4.13 (m, 2H), 4.12-3.95 (m, 3H), 3.95- 3.83 (m, 2H), 3.54-3.53 (m, 1H), 2.29-2.13 (m, 4H), 1.88-1.66 (m, 2H), 1.66-1.49 (m, 7H), 1.49- 1.34 (m, 2H), 1.30-1.13 (m, 3H). |
| 1-100 | 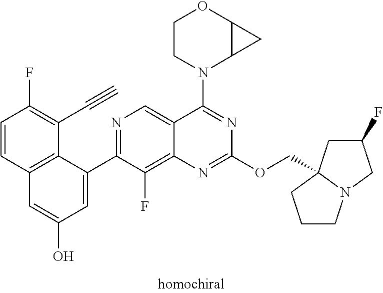 [SMILES: C#Cc1c(F)ccc2cc(O)cc(-c3ncc4c(N5CCOC6CC65)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{2-oxa-5- azabicyclo[4.1.0] heptan-5- yl}pyrido[4,3- d]pyrimidin-7-yl)- 5-ethynyl-6- fluoronaphthalen- 2-ol | 588.2 | ¹H NMR (400 MHz, DMSO-d₆) δ ppm = 10.35- 10.00 (m, 1H), 9.72 (d, J = 16.5 Hz, 1H), 7.98 (dd, J = 9.2, 5.9 Hz, 1H), 7.46 (td, J = 9.0, 1.4 Hz, 1H), 7.40 (d, J = 2.5 Hz, 1H), 7.20 (dd, J = 4.3, 2.6 Hz, 1H), 5.41-5.19 (m, 1H), 4.23- 3.85 (m, 7H), 3.55-3.45 (m, 2H), 3.10 (br d, J = 11.6 Hz, 3H), 2.89-2.80 (m, 1H), 2.16-1.99 (m, 3H), 1.88-1.74 (m, 3H), 1.29-1.14 (m, 2H). |
| 1-101 | 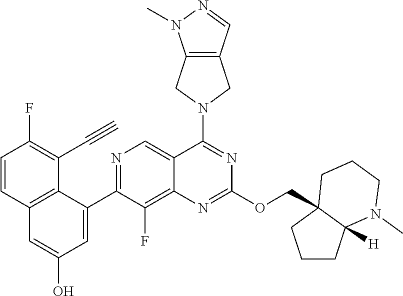 [SMILES: C#Cc1c(F)ccc2cc(O)cc(-c3ncc4c(N5Cc6cnn(C)c6C5)nc(OC[C@]56CCC[C@H]5N(C)CCC6)nc4c3F)c12] | 4-(2-{[(4aS,7aR)-1- methyl-octahydro- 1H-cyclopenta[b] pyridin-4a- yl]methoxy}- 8-fluoro-4-{1- methyl- 1H,4H,5H,6H- pyrrolo[3,4- c]pyrazol-5- yl}pyrido[4,3- d]pyrimidin-7-yl)- 5-ethynyl-6- fluoronaphthalen- 2-ol | 622.3 | |
| 1-102 | 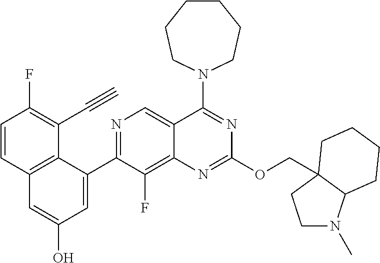 [SMILES: C#Cc1c(F)ccc2cc(O)cc(-c3ncc4c(N5CCCCCC5)nc(OCC56CCCCC5N(C)CC6)nc4c3F)c12] | 5-ethynyl-6-fluoro- 4-{8-fluoro-2-[(1- methyl-octahydro- 1H-indol-3a- yl)methoxy]-4- (1,4-oxazepan-4- yl)pyrido[4,3- d]pyrimidin-7- yl}naphthalen- 2-ol | 600.2 | |
| 1-103 | 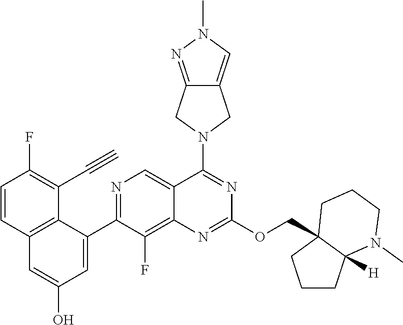 [SMILES: C#Cc1c(F)ccc2cc(O)cc(-c3ncc4c(N5Cc6cn(C)nc6C5)nc(OC[C@]56CCC[C@H]5N(C)CCC6)nc4c3F)c12] | 4-(2-{[(4aS,7aR)-1- methyl-octahydro- 1H-cyclopenta[b] pyridin-4a- yl]methoxy}- 8-fluoro-4-{2- methyl- 2H,4H,5H,6H- pyrrolo[3,4- c]pyrazol-5- yl}pyrido[4,3- d]pyrimidin-7-yl)- 5-ethynyl-6- fluoronaphthalen- 2-ol | 622.3 | |
| 1-104 | 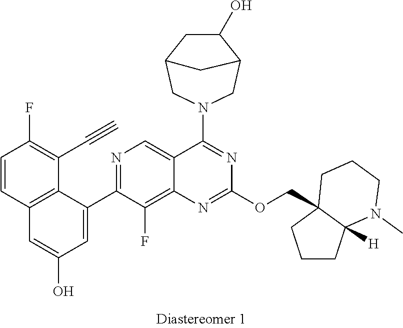 [SMILES: C#Cc1c(F)ccc2cc(O)cc(-c3ncc4c(N5CC6CC(O)C(C6)C5)nc(OC[C@]56CCC[C@H]5N(C)CCC6)nc4c3F)c12] | 3-(2-{[(4aS,7aR)-1- methyl-octahydro- 1H-cyclopenta[b] pyridin-4a- yl]methoxy}- 7-(8-ethynyl-7- fluoro-3- hydroxynaphthalen- 1-yl)-8- fluoropyrido[4,3- d]pyrimidin-4-yl)- 3-azabicyclo [3.2.1]octan-6-ol | 626.3 | ¹H NMR (400 MHz, DMSO-d₆) δ ppm = 9.39- 9.04 (m, 1H), 7.96 (dd, J = 9.1, 6.0 Hz, 1H), 7.44 (t, J = 9.0 Hz, 1H), 7.40-7.36 (m, 1H), 7.28-7.17 (m, 1H), 5.07-4.91 (m, 2H), 4.68-4.55 (m, 1H), 4.49- 4.35 (m, 2H), 4.18-4.10 (m, 2H), 3.92-3.86 (m, 1H), 3.76 (s, 1H), 3.68-3.62 (m, 1H), 2.65 (br s, 1H), 2.26- 2.15 (m, 5H), 2.13-2.06 (m, 1H), 1.86-1.78 (m, 2H), 1.65-1.51 (m, 8H), 1.43-1.35 (m, 1H), 1.30- 1.22 (m, 2H), 1.00-0.94 (m, 1H). |
| 1-105 | 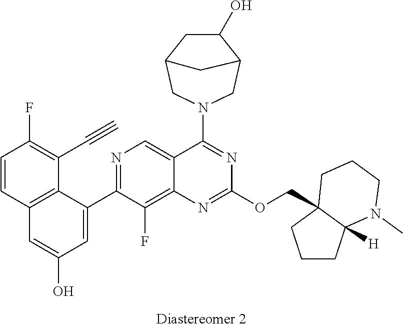 [SMILES: C#Cc1c(F)ccc2cc(O)cc(-c3ncc4c(N5CC6CC(O)C(C6)C5)nc(OC[C@]56CCC[C@H]5N(C)CCC6)nc4c3F)c12] | 3-(2-{[(4aS,7aR)-1- methyl-octahydro- 1H-cyclopenta[b] pyridin-4a- yl]methoxy}- 7-(8-ethynyl-7- fluoro-3- hydroxynaphthalen- 1-yl)-8- fluoropyrido[4,3- d]pyrimidin-4-yl)- 3-azabicyclo [3.2.1]octan-6-ol | 626.3 | ¹H NMR (400 MHz, DMSO-d₆) δ ppm = 9.39- 9.04 (m, 1H), 7.96 (dd, J = 9.1, 6.0 Hz, 1H), 7.44 (t, J = 9.0 Hz, 1H), 7.40-7.36 (m, 1H), 7.28-7.17 (m, 1H), 5.07-4.91 (m, 2H), 4.68- 4.55 (m, 1H), 4.49-4.35 (m, 2H), 4.18-4.10 (m, 2H), 3.92-3.86 (m, 1H), 3.76 (s, 1H), 3.68-3.62 (m, 1H), 2.65 (br s, 1H), 2.26-2.15 (m, 5H), 2.13- 2.06 (m, 1H), 1.86-1.78 (m, 2H), 1.65-1.51 (m, 8H), 1.43-1.35 (m, 1H), 1.30-1.22 (m, 2H), 1.00- 0.94 (m, 1H). |
| 1-106 | 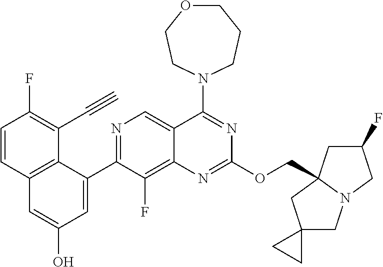 [SMILES: C#Cc1c(F)ccc2cc(O)cc(-c3ncc4c(N5CCCOCC5)nc(OC[C@@]56C[C@@H](F)CN5CC5(CC5)C6)nc4c3F)c12] | 4-(2-{[(6′R,7′aR)-6′- fluoro- hexahydrospiro [cyclopropane-1,2′- pyrrolizine]-7′a- yl]methoxy}-8- fluoro-4-(1,4- oxazepan-4- yl)pyrido[4,3- d]pyrimidin-7-yl)- 5-ethynyl-6- fluoronaphthalen- 2-ol | 616.2 | |
| 1-107 | 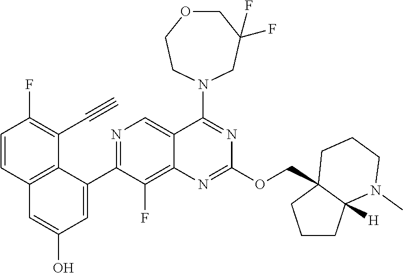 [SMILES: C#Cc1c(F)ccc2cc(O)cc(-c3ncc4c(N5CCOCC(F)(F)C5)nc(OC[C@]56CCC[C@H]5N(C)CCC6)nc4c3F)c12] | 4-(2-{[(4aS,7aR)-1- methyl-octahydro- 1H-cyclopenta[b] pyridin-4a- yl]methoxy}- 4-(6,6-difluoro-1,4- oxazepan-4-yl)-8- fluoropyrido[4,3- d]pyrimidin-7-yl)- 5-ethynyl-6- fluoronaphthalen- 2-ol | 636.15 | ¹H NMR (400 MHz, DMSO-d₆) δ ppm = 9.98- 10.31 (m, 1H), 9.11 (s, 1H), 7.98 (dd, J = 9.26, 6.00 Hz, 1H), 7.47 (t, J = 9.01 Hz, 1H), 7.40 (d, J = 2.50 Hz, 1H), 7.21 (d, J = 2.38 Hz, 1H), 4.88-5.00 (m, 1H), 4.32-4.59 (m, 2H), 4.04- 4.26 (m, 7H), 3.98 (s, 1H), 3.52 (br s, 1H), 2.60-2.66 (m, 1H), 2.54-2.71 (m, 3H), 1.78-2.01 (m, 1H), 1.66-1.78 (m, 1H), 1.49- 1.65 (m, 7H), 1.33-1.48 (m, 1H). |
| 1-108 | 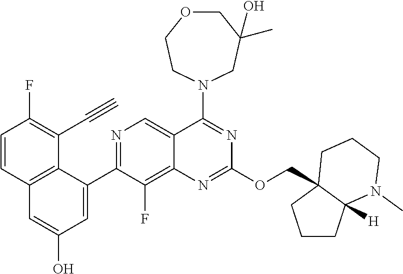 [SMILES: C#Cc1c(F)ccc2cc(O)cc(-c3ncc4c(N5CCOCC(C)(O)C5)nc(OC[C@]56CCC[C@H]5N(C)CCC6)nc4c3F)c12] | 4-(2-{[(4aS,7aR)-1- methyl-octahydro- 1H-cyclopenta[b] pyridin-4a- yl]methoxy}- 7-(8-ethynyl-7- fluoro-3- hydroxynaphthalen- 1-yl)-8- fluoropyrido[4,3- d]pyrimidin-4-yl)- 6-methyl-1,4- oxazepan-6-ol | 630.3 | ¹H NMR (400 MHz, DMSO-d₆) δ ppm = 9.46 - 9.28 (m, 1H), 7.97 (dd, J = 9.2, 5.9 Hz, 1H), 7.46 (t, J = 9.0 Hz, 1H), 7.39 (d, J = 2.4 Hz, 1H), 7.19 (dd, J = 10.7, 2.3 Hz, 1H), 5.29-5.05 (m, 1H), 4.50-4.30 (m, 2H), 4.21-3.82 (m, 7H), 3.63- 3.52 (m, 3H), 2.65-2.61 (m, 1H), 2.27-2.15 (m, 4H), 1.87-1.70 (m, 2H), 1.66-1.49 (m, 7H), 1.46- 1.36 (m, 1H), 1.17 (d, J = 8.0 Hz, 3H). |
| 1-109^{A} | 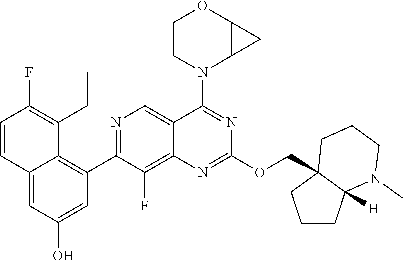 [SMILES: CCc1c(F)ccc2cc(O)cc(-c3ncc4c(N5CCOC6CC65)nc(OC[C@]56CCC[C@H]5N(C)CCC6)nc4c3F)c12] | 4-(4-(2-oxa-5- azabicyclo[4.1.0] heptan-5-yl)- 8-fluoro- 2-(((4aS,7aR)-1- methyloctahydro- 4aH-cyclopenta[b] pyridin-4a- yl)methoxy)pyrido [4,3-d]pyrimidin- 7-yl)-5-ethyl-6- fluoronaphthalen- 2-ol | 602.3 | ¹H NMR (400 MHz, DMSO-d₆) δ ppm = 9.94 (br s, 1H), 9.76 (d, J = 11.3 Hz, 1H), 7.77 (dd, J = 9.1, 6.0 Hz, 1H), 7.39-7.31 (m, 2H), 7.04 (t, J = 3.1 Hz, 1H), 4.50 (dd, J = 10.7, 4.3 Hz, 1H), 4.22-4.04 (m, 2H), 4.02- 3.83 (m, 3H), 3.53-3.44 (m, 2H), 2.70-2.62 (m, 1H), 2.26-2.13 (m, 5H), 1.87-1.78 (m, 1H), 1.72 (br dd, J = 8.6, 7.3 Hz, 1H), 1.66-1.46 (m, 8H), 1.43-1.37 (m, 1H), 1.28- |
| | | | | 1.20 (m, 2H), 1.12-1.03 |
| | | | | (m, 1H), 0.74 (q, J = |
| | | | | 7.2 Hz, 3H). |
| 1-110 | 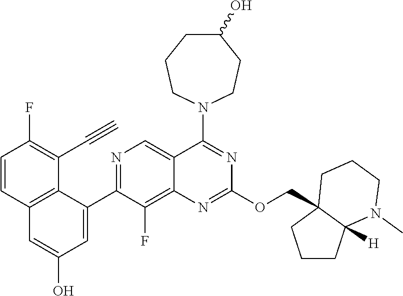 [SMILES: C#Cc1c(F)ccc2cc(O)cc(-c3ncc4c(N5CCCC(O)CC5)nc(OC[C@]56CCC[C@H]5N(C)CCC6)nc4c3F)c12] | 1-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)azepan-4-ol | 614.2 | ¹H NMR (400 MHz, DMSO-d₆) δ ppm = 10.21 (brs, 1H), 9.07 (s, 1H), 7.97 (dd, J = 9.1, 5.9 Hz, 1H), 7.46 (t, J = 9.0 Hz, 1H), 7.39 (d, J = 2.5 Hz, 1H), 7.18 (t, J = 2.3 Hz, 1H), 4.67 (br d, J = 2.5 Hz, 1H), 4.50-4.37 (m, 1H), 4.22- 4.11 (m, 1H), 4.07-3.80 (m, 5H), 2.69-2.62 (m, 1H), 2.47-2.41 (m, 1H), 2.25-2.01 (m, 6H), 1.96- 1.69 (m, 5H), 1.67-1.38 (m, 10H). (Diastereomeric mixture) |
| 1-111 | 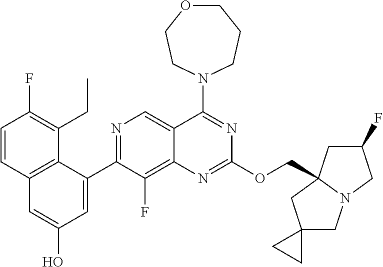 [SMILES: CCc1c(F)ccc2cc(O)cc(-c3ncc4c(N5CCCOCC5)nc(OC[C@@]56C[C@@H](F)CN5CC5(CC5)C6)nc4c3F)c12] | 5-ethyl-6-fluoro-4- (8-fluoro-2- (((6′R,7a′R)-6′- fluorodihydro- 1′H,3′H-spiro [cyclopropane- 1,2′-pyrrolizin]- 7a′(5′H)- yl)methoxy)-4- (1,4-oxazepan-4- yl)pyrido[4,3- d]pyrimidin-7- yl)naphthalen- 2-ol | 620.3 | ¹H NMR (400 MHz, DMSO-d₆) δ ppm = 9.95 (s, 1H), 9.18 (s, 1H), 7.78 (dd, J = 8.9, 6.0 Hz, 1H), 7.38- 7.32 (m, 2H), 7.04 (s, 1H), 5.59-5.30 (m, 1H), 4.41- 4.32 (m, 1H), 4.22-4.12 (m, 6H), 3.98-3.91 (m, 2H), 3.79-3.72 (m, 2H), 3.13-2.91 (m, 1H), 2.85- 2.80 (m, 1H), 2.70-2.61 (m, 2H), 2.41-2.30 (m, 2H), 2.14-2.04 (m, 5H), 0.74 (t, J = 7.3 Hz, 3H), 0.58-0.47 (m, 4H). |
| 1-112^{H} | 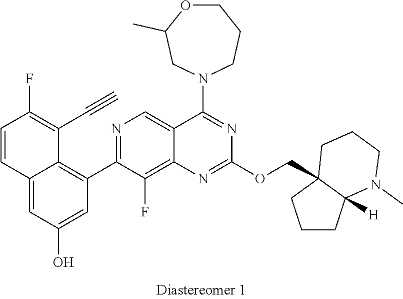 [SMILES: C#Cc1c(F)ccc2cc(O)cc(-c3ncc4c(N5CCCOC(C)C5)nc(OC[C@]56CCC[C@H]5N(C)CCC6)nc4c3F)c12] | 5-ethynyl-6-fluoro- 4-(8-fluoro-4-(2- methyl-1,4- oxazepan-4-yl)-2- (((4aS,7aR)-1- methyloctahydro- 4aH-cyclopenta[b] pyridin-4a- yl)methoxy)pyrido [4,3-d]pyrimidin- 7-yl)naphthalen- 2-ol | 614.3 | ¹H NMR (400 MHz, DMSO-d₆) δ ppm = 10.19 (s, 1H), 9.09 (d, J = 13.3 Hz, 1H), 7.98 (dd, J = 9.0, 6.5 Hz, 1H), 7.47 (t, J = 9.0 Hz, 1H), 7.40 (d, J = 2.5 Hz, 1H), 7.18 (dd, J= 13.1, 2.1 Hz, 1H), 4.53-4.34 (m, 1H), 4.20-4.08 (m, 3H), 4.05-3.90 (m, 3H), 3.54 (br d, J = 9.5 Hz, 1H), 2.29-2.09 (m, 7H), 1.87- 1.68 (m, 2H), 1.68-1.47 (m, 8H), 1.46-1.35 (m, 1H), 1.27-1.23 (m, 5H). |
| 1-113^{H} | 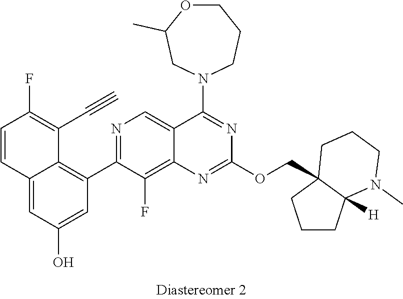 [SMILES: C#Cc1c(F)ccc2cc(O)cc(-c3ncc4c(N5CCCOC(C)C5)nc(OC[C@]56CCC[C@H]5N(C)CCC6)nc4c3F)c12] | 5-ethynyl-6-fluoro- 4-(8-fluoro-4-(2- methyl-1,4- oxazepan-4-yl)-2- (((4aS,7aR)-1- methyloctahydro- 4aH-cyclopenta[b] pyridin-4a- yl)methoxy)pyrido [4,3-d]pyrimidin- 7-yl)naphthalen- 2-ol | 614.2 | ¹H NMR (400 MHz, DMSO-d₆) δ ppm = 10.16 (s, 1H), 9.09 (d, J = 13.5 Hz, 1H), 8.01-7.94 (m, 1H), 7.47 (t, J = 9.1 Hz, 1H), 7.40 (d, J = 2.5 Hz, 1H), 7.21-7.14 (m, 1H), 4.50-4.31 (m,1H), 4.22- 4.08 (m, 3H), 4.03-3.93 (m, 3H), 3.59-3.52 (m, 3H), 2.29-2.09 (m, 4H), 1.76-1.49 (m, 12H), 1.27- 1.23 (m, 5H). |
| 1-114 | 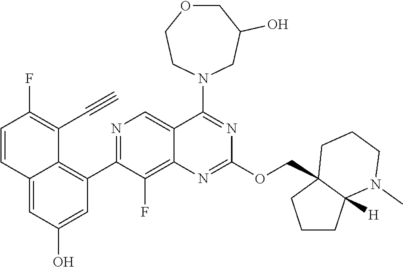 [SMILES: C#Cc1c(F)ccc2cc(O)cc(-c3ncc4c(N5CCOCC(O)C5)nc(OC[C@]56CCC[C@H]5N(C)CCC6)nc4c3F)c12] | 4-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-1,4- oxazepan-6- ol | 616.2 | ¹H NMR (400 MHz, DMSO-d₆) δ ppm = 10.5- 10.1 (brs, 1H), 9.31-9.07 (m, 1H), 7.98 (dd, J = 8.8, 5.8 Hz, 1H), 7.51-7.43 (m, 1H), 7.40 (d, J = 2.5 Hz, 1H), 7.20 (dd, J = 6.9, 2.5 Hz, 1H), 5.40 (br s, 1H), 4.47 (dd, J = 10.6, 5.9 Hz, 1H), 4.39-4.21 (m, 1H), 4.20-4.05 (m, 3H), 4.03- 3.80 (m, 4H), 3.64-3.59 (m, 1H), 2.68-2.64 (m, 1H), 2.48-2.44 (m, 1H), 2.26-2.15 (m, 4H), 1.82- 1.33 (m, 12H). |
| 1-115 | 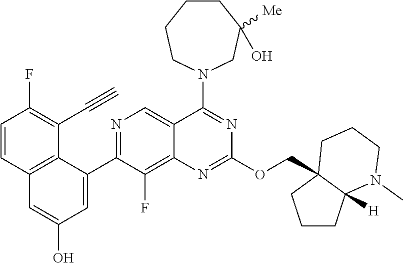 [SMILES: C#Cc1c(F)ccc2cc(O)cc(-c3ncc4c(N5CCCCC(C)(O)C5)nc(OC[C@]56CCC[C@H]5N(C)CCC6)nc4c3F)c12] | 1-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-3- methylazepan- 3-ol | 628.3 | ¹H NMR (400 MHz, DMSO-d₆) δ ppm = 10.18 (s, 1H), 9.47-9.17 (m, 1H), 7.97 (dd, J = 9.0, 6.0 Hz, 1H), 7.52-7.43 (m, 1H), 7.39 (d, J = 2.5 Hz, 1H), 7.19 (dd, J = 12.3, 2.5 Hz, 1H), 4.99-4.74 (m, 1H), 4.50-4.33 (m, 1H), 4.22- 3.93 (m, 3H), 3.91-3.68 (m, 1H), 2.67-2.59 (m, 1H), 2.28-2.14 (m, 4H), 2.12-2.02 (m, 1H), 1.91- 1.77 (m, 4H), 1.74-1.47 (m, 13H), 1.45-1.35 (m, 1H), 1.28 (s, 3H). (Mixture of diastereomers) |
| 1-116 | 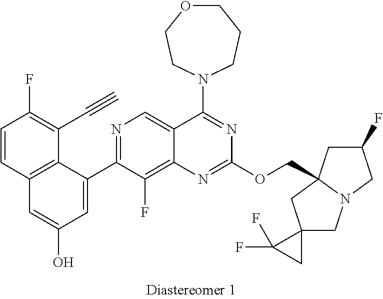 [SMILES: C#Cc1c(F)ccc2cc(O)cc(-c3ncc4c(N5CCCOCC5)nc(OC[C@@]56C[C@@H](F)CN5CC5(CC5(F)F)C6)nc4c3F)c12] | 5-ethynyl-6-fluoro- 4-(8-fluoro-4-(1,4- oxazepan-4-yl)-2- (((6′R,7a′R)-2,2,6′- trifluorodihydro- 1′H,3′H-spiro [cyclopropane- 1,2′-pyrrolizin]- 7a′(5′H)- yl)methoxy)pyrido [4,3-d]pyrimidin- 7-yl)naphthalen- 2-ol | 652.2 | ¹H NMR (400 MHz, DMSO-d6) 0 ppm = 10.18 (br s, 1H), 9.11 (s, 1H), 7.98 (dd, J = 9.1, 5.9 Hz, 1H), 7.47 (t, J = 9.0 Hz, 1H), 7.40 (d, J = 2.8 Hz, 1H), 7.19 (d, J = 2.5 Hz, 1H), 5.50-5.26 (m, 1H), 4.37-4.30 (m, 1H), 4.29- 4.24 (m, 1H), 4.20-4.04 (m, 4H), 4.00 (d, J = 2.5 Hz, 1H), 3.95 (t, J = 4.5 Hz, 2H), 3.82-3.69 (m, 2H), 3.20-3.12 (m, 1H), 2.89- 2.69 (m, 2H), 2.38-2.26 (m, 1H), 2.20-1.92 (m, 6H), 1.67-1.46 (m, 2H). |
| 1-117 | 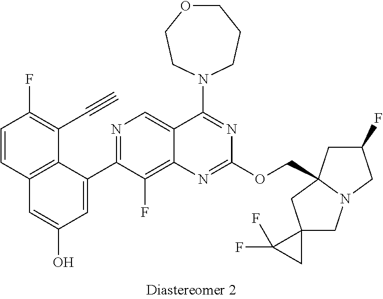 [SMILES: C#Cc1c(F)ccc2cc(O)cc(-c3ncc4c(N5CCCOCC5)nc(OC[C@@]56C[C@@H](F)CN5CC5(CC5(F)F)C6)nc4c3F)c12] | 5-ethynyl-6-fluoro- 4-(8-fluoro-4-(1,4- oxazepan-4-yl)-2- (((6′R,7a′R)-2,2,6′- trifluorodihydro- 1′H,3′H-spiro [cyclopropane- 1,2′-pyrrolizin]- 7a′(5′H)- yl)methoxy)pyrido [4,3-d]pyrimidin- 7-yl)naphthalen- 2-ol | 652.2 | ¹H NMR (400 MHz, DMSO-d₆) δ ppm = 10.39 (br s, 1H), 9.11 (s, 1H), 7.98 (dd, J = 8.9, 6.0 Hz, 1H), 7.47 (t, J = 8.9 Hz, 1H), 7.39 (s, 1H), 7.22- 7.16 (m, 1H), 5.59-5.30 (m, 1H), 4.23-4.10 (m, 5H), 4.01 (s, 2H), 3.96- 3.90 (m, 2H), 3.80-3.73 (m, 2H), 3.15-2.92 (m, 2H), 2.84-2.73 (m, 1H), 2.40-2.26 (m, 1H), 2.23- 2.06 (m, 5H), 2.02-1.93 (m, 1H), 1.64-1.52 (m, 2H). |
| 1-118 | 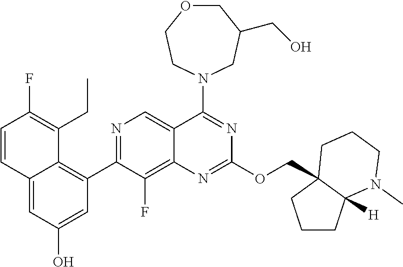 [SMILES: CCc1c(F)ccc2cc(O)cc(-c3ncc4c(N5CCOCC(CO)C5)nc(OC[C@]56CCC[C@H]5N(C)CCC6)nc4c3F)c12] | 5-ethyl-6-fluoro-4- (8-fluoro-4-(6- (hydroxymethyl)- 1,4-oxazepan-4-yl)- 2-(((4aS,7aR)-1- methyloctahydro- 4aH-cyclopenta[b] pyridin-4a- yl)methoxy)pyrido [4,3-d]pyrimidin- 7-yl)naphthalen- 2-ol | 634.3 | ¹H NMR (400 MHz, DMSO-d₆) δ ppm = 9.43 (s, 1H), 9.21 (d, J = 2.5 Hz, 1H), 7.77 (dd, J = 9.1, 5.9 Hz, 1H), 7.42-7.26 (m, 2H), 7.03 (dd, J = 10.0, 2.5 Hz, 1H), 4.62-4.42 (m, 2H), 4.33-4.23 (m, 1H), 4.17 (t, J = 10.5 Hz, 1H), 4.07-3.82 (m, 4H), 3.76- 3.68 (m, 2H), 3.56-3.50 (m, 2H), 2.65 (br d, J = 2.5 Hz, 1H), 2.47-2.33 (m, 3H), 2.25-2.11 (m, 6H), 1.85-1.68 (m, 2H), 1.66- 1.46 (m, 7H), 1.44-1.35 (m, 1H), 0.76-0.72 (m, 3H). |
| 1-119^{I} | 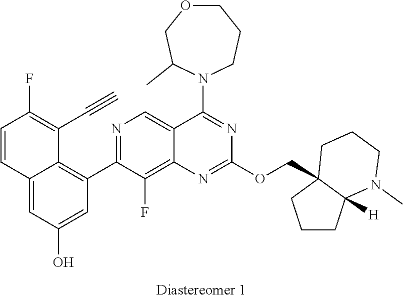 [SMILES: C#Cc1c(F)ccc2cc(O)cc(-c3ncc4c(N5CCCOCC5C)nc(OC[C@]56CCC[C@H]5N(C)CCC6)nc4c3F)c12] | 5-ethynyl-6-fluoro- 4-(8-fluoro-4-(3- methyl-1,4- oxazepan-4-yl)-2- (((4aS,7aR)-1- methyloctahydro- 4aH-cyclopenta[b] pyridin-4a- yl)methoxy)pyrido [4,3-d]pyrimidin- 7-yl)naphthalen- 2-ol | 614.3 | ¹H NMR (400 MHz, DMSO-d₆) δ ppm = 9.03 (d, J = 11.0 Hz, 1H), 8.22 (br s, 1H), 8.03-7.89 (m, 1H), 7.47 (t, J = 9.1 Hz, 1H), 7.41-7.35 (m, 1H), 7.23-7.07 (m, 1H), 4.86 (br s, 1H), 4.49 (dd, J = 15.4, 10.6 Hz, 1H), 4.42- 4.26 (m, 1H), 4.21-4.03 (m, 2H), 4.01-3.93 (m, 1H), 3.87-3.78 (m, 1H), 3.66-3.60 (m, 2H), 2.69- 2.64 (m, 1H), 2.29-2.12 (m, 5H), 2.09-1.96 (m, 1H), 1.87-1.68 (m, 1H), 1.67-1.45 (m, 7H), 1.41- 1.11 (m, 7H). |
| 1-120^{J} | 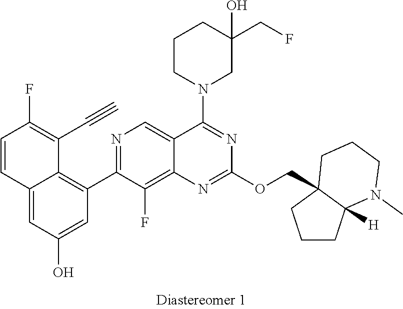 [SMILES: C#Cc1c(F)ccc2cc(O)cc(-c3ncc4c(N5CCCC(O)(CF)C5)nc(OC[C@]56CCC[C@H]5N(C)CCC6)nc4c3F)c12] | 1-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-3- (fluoromethyl) piperidin-3-ol | 632.2 | ¹H NMR (400 MHz, DMSO-d₆) δ ppm = 10.27- 10.09 (m, 1H), 9.23-9.04 (m, 1H), 7.98 (dd, J = 9.1, 5.9 Hz, 1H), 7.51-7.43 (m, 1H), 7.40 (d, J = 2.1 Hz, 1H), 7.22 (dd, J = 16.5, 2.4 Hz, 1H), 5.27-5.16 (m, 1H), 4.49 (dd, J = 10.9, 2.4 Hz, 1H), 4.43-4.37 (m, 1H), 4.35-4.25 (m, 1H), 4.24-4.12 (m, 2H), 4.05 (br d, J = 13.4 Hz, 1H), 3.92 (s, 1H), 3.81-3.66 (m, 2H), 2.76-2.72 (m, 1H), 2.32-2.21 (m, 1H), 2.22 (s, 3H), 2.13-2.00 (m, 1H), 1.87-1.40 (m, 14H). |
| 1-121^{J} | 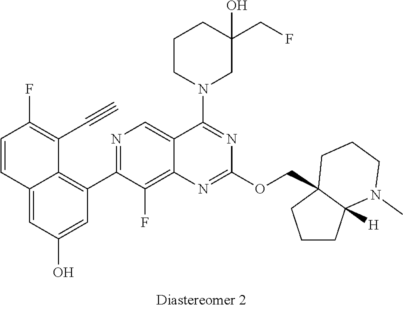 [SMILES: C#Cc1c(F)ccc2cc(O)cc(-c3ncc4c(N5CCCC(O)(CF)C5)nc(OC[C@]56CCC[C@H]5N(C)CCC6)nc4c3F)c12] | 1-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-3- (fluoromethyl) piperidin-3-ol | 632.2 | ¹H NMR (400 MHz, DMSO-d₆) δ ppm = 10.34- 10.04 (m, 1H), 9.23-9.04 (m, 1H), 7.98 (dd, J = 9.2, 5.9 Hz, 1H), 7.47 (td, J = 9.1, 1.5 Hz, 1H), 7.40 (d, J = 2.4 Hz, 1H), 7.22 (dd, J = 15.7, 2.4 Hz, 1H), 5.28-5.15 (m, 1H), 4.50- 4.26 (m, 3H), 4.24-4.13 (m, 2H), 4.04 (br d, J = 13.6 Hz, 1H), 3.95-3.90 (m, 1H), 3.81-3.66 (m, 2H), 2.71 (br t, J = 5.4 Hz, 1H), 2.31-2.19 (m, 1H), 2.20 (s, 3H), 2.13-2.01 (m, 1H), 1.87-1.50 (m, 14H). |
| 1-122 | 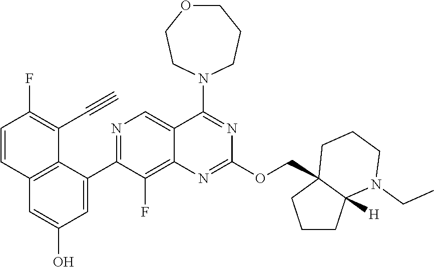 [SMILES: C#Cc1c(F)ccc2cc(O)cc(-c3ncc4c(N5CCCOCC5)nc(OC[C@]56CCC[C@H]5N(CC)CCC6)nc4c3F)c12] | 4-(2-(((4aS,7aR)-1- ethyloctahydro- 4aH-cyclopenta[b] pyridin-4a- yl)methoxy)- 8-fluoro-4-(1,4- oxazepan-4- yl)pyrido[4,3- d]pyrimidin-7-yl)- 5-ethynyl-6- fluoronaphthalen- 2-ol | 614.3 | ¹H NMR (400 MHz, DMSO-d₆) δ ppm = 10.16 (br s, 1H), 9.10 (s, 1H), 7.98 (dd, J = 9.1, 5.9 Hz, 1H), 7.47 (t, J = 9.0 Hz, 1H), 7.40 (d, J = 2.5 Hz, 1H), 7.19 (d, J = 2.5 Hz, 1H), 4.63-4.52 (m, 1H), 4.26-4.05 (m, 6H), 4.01 (dd, J = 5.3, 1.0 Hz, 1H), 3.95 (t, J = 4.8 Hz, 2H), 3.84-3.72 (m, 2H), 2.98 (br s, 1H), 2.41 (br dd, J = 5.4, 1.6 Hz, 2H), 2.16-2.08 (m, 2H), 1.89-1.80 (m, 1H), 1.77-1.68 (m, 1H), 1.67-1.42 (m, 8H), 1.41- |
| | | | | 1.28 (m, 1H), 1.05-0.96 |
| | | | | (m, 3H). |
| 1-123 | 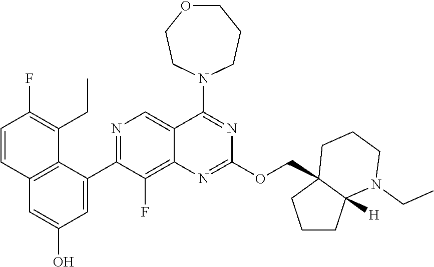 [SMILES: CCc1c(F)ccc2cc(O)cc(-c3ncc4c(N5CCCOCC5)nc(OC[C@]56CCC[C@H]5N(CC)CCC6)nc4c3F)c12] | 5-ethyl-4-(2- (((4aS,7aR)-1- ethyloctahydro- 4aH-cyclopenta[b] pyridin-4a-yl) methoxy)- 8-fluoro-4-(1,4- oxazepan-4- yl)pyrido[4,3- d]pyrimidin-7-yl)- 6-fluoronaphthalen- 2-ol | 618.3 | ¹H NMR (400 MHz, DMSO-d₆) ′δ ppm = 9.96 (br s, 1H), 9.16 (s, 1H), 7.77 (dd, J = 9.3, 6.0 Hz, 1H), 7.42-7.27 (m, 2H), 7.03 (d, J = 2.5 Hz, 1H), 4.59 (dd, J = 10.8, 4.8 Hz, 1H), 4.23 (dd, J = 10.8, 2.3 Hz, 1H), 4.19-4.09 (m, 4H), 3.95 (t, J = 4.8 Hz, 2H), 3.75 (t, J = 5.1 Hz, 2H), 2.99 (br s, 1H), 2.48-2.34 (m, 4H), 2.22-2.05 (m, 3H), 1.86 (br dd, J = 10.8, 2.3 Hz, 1H), 1.78-1.67 (m, 1H), 1.66- 1.42 (m, 8H), 1.40-1.29 (m, 1H), 0.98 (dt, J = 7.1, 3.3 Hz, 3H), 0.81-0.65 (m, 3H). |
| 1-124^{K} | 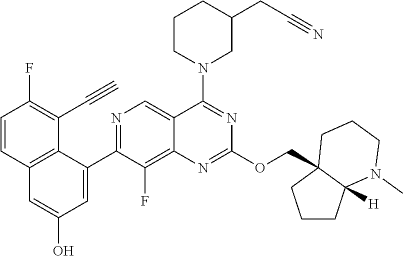 [SMILES: C#Cc1c(F)ccc2cc(O)cc(-c3ncc4c(N5CCCC(CC#N)C5)nc(OC[C@]56CCC[C@H]5N(C)CCC6)nc4c3F)c12] | 2-(1-(7-(8-ethynyl- 7-fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)piperidin-3- yl)acetonitrile | 623.3 | ¹H NMR (400 MHz, DMSO-d₆) δ ppm = 10.18 (br s, 1H), 9.00 (d, J = 9.3 Hz, 1H), 7.98 (dd, J = 8.9, 6.0 Hz, 1H), 7.47 (t, J = 8.9 Hz, 1H), 7.40 (d, J = 2.5 Hz, 1H), 7.20 (d, J = 1.0 Hz, 1H), 4.54-4.32 (m, 3H), 4.17-4.13 (m, 1H), 3.98 (s, 1H), 3.17-3.15 (m, 1H), 2.76-2.61 (m, 3H), 2.44 (br s, 1H), 2.29-2.11 (m, 5H), 2.05-1.69 (m, 6H), 1.66-1.34 (m, 9H). |
| 1-125^{B} | 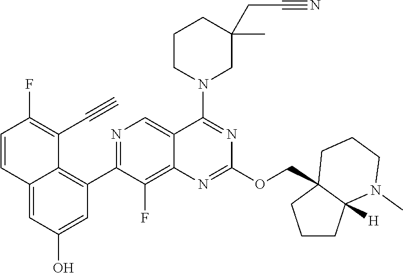 [SMILES: C#Cc1c(F)ccc2cc(O)cc(-c3ncc4c(N5CCCC(C)(CC#N)C5)nc(OC[C@]56CCC[C@H]5N(C)CCC6)nc4c3F)c12] | 2-(1-(7-(8-ethynyl- 7-fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-3- methylpiperidin-3- yl)acetonitrile | 636.75 | |
| 1-126 | 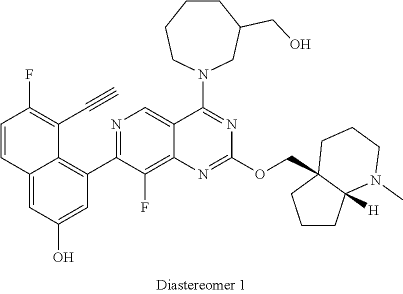 [SMILES: C#Cc1c(F)ccc2cc(O)cc(-c3ncc4c(N5CCCCC(CO)C5)nc(OC[C@]56CCC[C@H]5N(C)CCC6)nc4c3F)c12] | 5-ethynyl-6-fluoro- 4-(8-fluoro-4-(3- (hydroxymethyl) azepan-1-yl)-2- (((4aS,7aR)-1- methyloctahydro- 4aH-cyclopenta[b] pyridin-4a- yl)methoxy)pyrido [4,3-d]pyrimidin- 7-yl)naphthalen- 2-ol | 628.3 | ¹H NMR (400 MHz, DMSO-d₆) δ ppm = 9.04 (s, 1H), 7.95 (dd, J = 9.3, 6.0 Hz, 1H), 7.45 (t, J = 9.0 Hz, 1H), 7.38 (d, J = 2.5 Hz, 1H), 7.19 (d, J = 2.5 Hz, 1H), 4.62-4.51 (m, 1H), 4.49-4.36 (m, 2H), 4.13 (d, J = 10.8 Hz, 1H), 3.95- 3.88 (m, 1H), 3.59 (br dd, J = 12.1, 7.1 Hz, 1H), 3.53- 3.50 (m, 2H), 2.68-2.62 (m, 1H), 2.48-2.35 (m, 2H), 2.25-2.19 (m, 1H), 2.17 (s, 3H), 1.87-1.76 (m, 2H), 1.73-1.47 (m, 17H). |
| 1-127 | 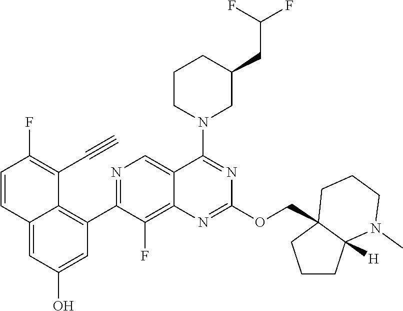 [SMILES: C#Cc1c(F)ccc2cc(O)cc(-c3ncc4c(N5CCC[C@@H](CC(F)F)C5)nc(OC[C@]56CCC[C@H]5N(C)CCC6)nc4c3F)c12] | 4-(4-((S)-3-(2,2- difluoroethyl) piperidin-1-yl)- 8-fluoro- 2-(((4aS,7aR)-1- methyloctahydro- 4aH-cyclopenta[b] pyridin-4a- yl)methoxy)pyrido [4,3-d]pyrimidin-7- yl)-5-ethynyl-6- fluoronaphthalen- 2-ol | 648.3 | ¹H NMR (400 MHz, DMSO-d₆) δ ppm = 10.26- 10.11 (m, 1H), 8.98 (d, J = 2.4 Hz, 1H), 7.98 (dd, J = 9.2, 5.9 Hz, 1H), 7.47 (t, J = 9.0 Hz, 1H), 7.40 (d, J = 2.5 Hz, 1H), 7.20 (t, J = 2.5 Hz, 1H), 6.23 (tt, J = 56.2, 2.5 Hz, 1H), 4.52-4.37 (m, 3H), 4.14 (dd, J = 10.8, 8.0 Hz, 1H), 3.97 (dd, J = 2.8, 0.8 Hz, 1H), 3.23-3.12 (m, 2H), 2.30-2.03 (m, 4H), 2.01-1.69 (m, 9H), 1.68- 1.32 (m, 10H). |
| 1-128^{I} | 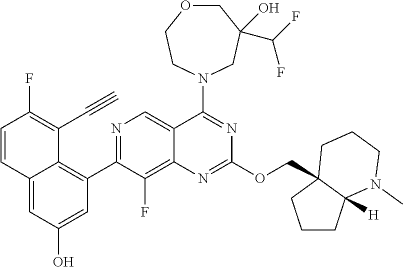 [SMILES: C#Cc1c(F)ccc2cc(O)cc(-c3ncc4c(N5CCOCC(O)(C(F)F)C5)nc(OC[C@]56CCC[C@H]5N(C)CCC6)nc4c3F)c12] | 6-(difluoromethyl)- 4-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-1,4-oxazepan- 6-ol | 666.3 | |
| 1-129 | 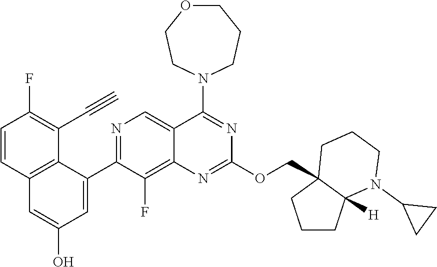 [SMILES: C#Cc1c(F)ccc2cc(O)cc(-c3ncc4c(N5CCCOCC5)nc(OC[C@]56CCC[C@H]5N(C5CC5)CCC6)nc4c3F)c12] | 4-(2-(((4aS,7aR)-1- cyclopropylocta- hydro-4aH- cyclopenta[b] pyridin-4a- yl)methoxy)- 8-fluoro-4-(1,4- oxazepan-4- yl)pyrido[4,3- d]pyrimidin-7-yl)- 5-ethynyl-6- fluoronaphthalen- 2-ol | 626.3 | ¹H NMR (400 MHz, DMSO-d₆) δ ppm = 10.16 (br s, 1H), 9.09 (s, 1H), 7.98 (dd, J = 9.3, 6.0 Hz, 1H), 7.47 (t, J = 9.0 Hz, 1H), 7.40 (d, J = 2.5 Hz, 1H), 7.18 (s, 1H), 4.48 (d, J = 10.5 Hz, 1H), 4.23- 4.06 (m, 5H), 4.03-3.96 (m, 1H), 3.96-3.87 (m, 2H), 3.77 (t, J = 5.1 Hz, 2H), 3.08-2.95 (m, 1H), 2.64-2.56 (m, 1H), 2.18- 1.95 (m, 3H), 1.79-1.71 (m, 2H), 1.65-1.31 (m, 9H), 0.47-0.32 (m, 2H), |
| | | | | 0.31-0.20 (m, 1H), 0.19- |
| | | | | 0.11 (m, 1H). |
| 1-130^{B} | 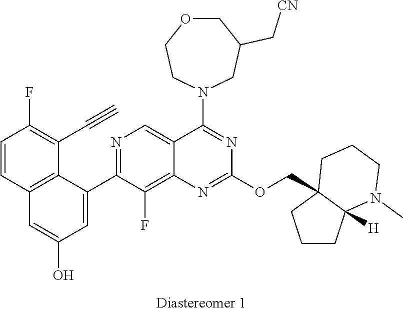 [SMILES: C#Cc1c(F)ccc2cc(O)cc(-c3ncc4c(N5CCOCC(CC#N)C5)nc(OC[C@]56CCC[C@H]5N(C)CCC6)nc4c3F)c12] | 2-(4-(7-(8-ethynyl- 7-fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-1,4-oxazepan- 6-yl)acetonitrile | 639.2 | ¹H NMR (400 MHz, DMSO-d₆) δ ppm = 10.20 (s, 1H), 9.11 (d, J = 10.2 Hz, 1H), 7.98 (dd, J = 9.0, 6.0 Hz, 1H), 7.47 (t, J = 9.0 Hz, 1H), 7.40 (s, 1H), 7.18 (dd, J = 12.0, 2.0 Hz, 1H), 4.57-4.23 (m, 2H), 4.20- 4.05 (m, 1H), 4.03-3.93 (m, 2H), 3.92-3.78 (m, 2H), 3.64-3.57 (m, 1H), 2.91-2.82 (m, 1H), 2.70- 2.66 (m, 4H), 2.29-2.14 (m, 4H), 1.87-1.69 (m, 2H), 1.67-1.44 (m, 6H), 1.43-1.35 (m, 1H), 1.24 (s, 2H), 1.15 (br t, J = 7.1 Hz, 2H). |
| 1-131^{B} | 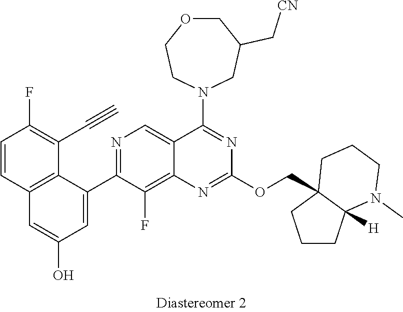 [SMILES: C#Cc1c(F)ccc2cc(O)cc(-c3ncc4c(N5CCOCC(CC#N)C5)nc(OC[C@]56CCC[C@H]5N(C)CCC6)nc4c3F)c12] | 2-(4-(7-(8-ethynyl- 7-fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-1,4- oxazepan-6- yl)acetonitrile | 639.2 | ¹H NMR (400 MHz, DMSO-d₆) δ ppm = 10.16 (br s, 1H), 9.12 (d, J = 9.7 Hz, 1H), 8.02-7.91 (m, 1H), 7.47 (t, J = 9.1 Hz, 1H), 7.40 (d, J = 2.5 Hz, 1H), 7.24-7.07 (m, 1H), 4.56-4.25 (m, 3H), 4.24- 4.16 (m, 1H), 4.14-3.81 (m, 4H), 3.64-3.58 (m, 1H), 2.71-2.62 (m, 4H), 2.29-2.14 (m, 4H), 1.87- 1.69 (m, 2H), 1.66-1.48 (m, 6H), 1.46-1.39 (m, 1H), 1.31-1.09 (m, 4H). |
| 1-132^{B} | 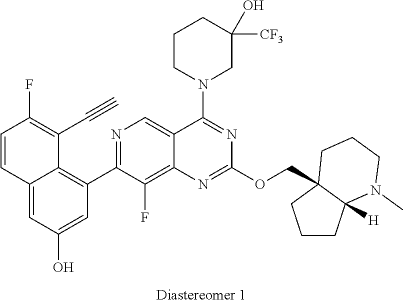 [SMILES: C#Cc1c(F)ccc2cc(O)cc(-c3ncc4c(N5CCCC(O)(C(F)(F)F)C5)nc(OC[C@]56CCC[C@H]5N(C)CCC6)nc4c3F)c12] | 1-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-3- (trifluoromethyl) piperidin-3-ol | 668.2 | ¹H NMR (400 MHz, DMSO-d₆) δ ppm = 10.18 (br s, 1H), 9.25-8.92 (m, 1H), 8.03-7.87 (m, 1H), 7.50-7.40 (m, 1H), 7.43- 7.38 (m, 1H), 7.27-7.13 (m, 1H), 6.41 (d, J = 4.5 Hz, 1H), 4.59-4.32 (m, 3H), 4.24-4.12 (m, 1H), 4.00-3.77 (m, 1H), 3.76- 3.65 (m, 1H), 2.76 (br s, 1H), 2.37-2.31 (m, 2H), 2.28 - 2.07 (m, 3H), 1.99- 1.92 (m, 1H), 1.88-1.71 (m, 2H), 1.69-1.36 (m, 12H). |
| 1-133^{B} | 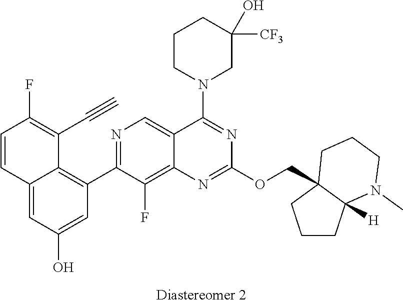 [SMILES: C#Cc1c(F)ccc2cc(O)cc(-c3ncc4c(N5CCCC(O)(C(F)(F)F)C5)nc(OC[C@]56CCC[C@H]5N(C)CCC6)nc4c3F)c12] | 1-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-3- (trifluoromethyl) piperidin-3-ol | 668.2 | ¹H NMR (400 MHz, DMSO-d₆) δ ppm = 10.17 (br s, 1H), 9.26-8.94 (m, 1H), 8.03-7.93 (m, 1H), 7.50-7.40 (m, 1H), 7.41 (t, J = 2.4 Hz, 1H), 7.28-7.18 (m, 1H), 6.44-6.38 (m, 1H), 4.58-4.32 (m, 3H), 4.18 (d, J = 10.8 Hz, 1H), 3.98-3.78 (m, 1H), 3.74- 3.70 (m, 1H), 2.78 (br s, 1H), 2.37-2.30 (m, 2H), 2.29-2.06 (m, 3H), 2.02- 1.93 (m, 1H), 1.98-1.85 (m, 2H), 1.87-1.35 (m, 12H). |
| 1-135^{G} | 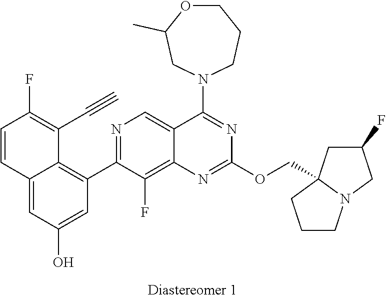 [SMILES: C#Cc1c(F)ccc2cc(O)cc(-c3ncc4c(N5CCCOC(C)C5)nc(OC[C@@]56CCCN5C[C@H](F)C6)nc4c3F)c12] | 5-ethynyl-6-fluoro- 4-(8-fluoro-2- (((2R,7aS)-2- fluorotetrahydro- 1H-pyrrolizin- 7a(5H)- yl)methoxy)-4-(2- methyl-1,4- oxazepan-4- yl)pyrido[4,3- d]pyrimidin-7- yl)naphthalen-2-ol | 604.2 | ¹H NMR (400 MHz, DMSO-d₆) δ ppm = 9.09 (d, J= 13.5 Hz, 1H), 7.96 (dd, J = 9.4, 5.9 Hz, 1H), 7.46 (t, J = 9.0 Hz, 1H), 7.39 (d, J = 2.5 Hz, 1H), 7.19 (dd, J = 12.6, 2.5 Hz, 1H), 5.41- 5.19 (m, 1H), 4.51-4.33 (m, 1H), 4.18-4.08 (m, 3H), 4.07-3.93 (m, 4H), 3.58-3.53 (m, 2H), 3.14- 3.08 (m, 2H), 2.88-2.80 (m, 1H), 2.24-1.93 (m, 6H), 1.91-1.66 (m, 4H), 1.27-1.20 (m, 3H). |
| 1-136^{A} | 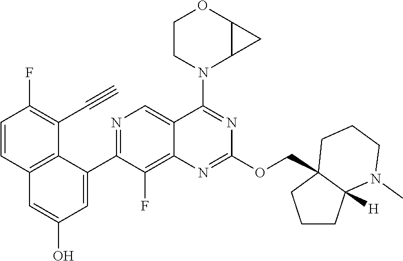 [SMILES: C#Cc1c(F)ccc2cc(O)cc(-c3ncc4c(N5CCOC6CC65)nc(OC[C@]56CCC[C@H]5N(C)CCC6)nc4c3F)c12] | 4-(4-(2-oxa-5- azabicyclo[4.1.0] heptan-5-yl)- 8-fluoro- 2-(((4aS,7aR)-1- methyloctahydro- 4aH-cyclopenta[b] pyridin-4a- yl)methoxy)pyrido [4,3-d]pyrimidin- 7-yl)-5-ethynyl-6- fluoronaphthalen- 2-ol | 598.3 | ¹H NMR (400 MHz, DMSO-d₆) δ ppm = 10.20- 10.13 (m, 1H), 9.72 (d, J = 15.6 Hz, 1H), 7.98 (dd, J = 9.2, 5.9 Hz, 1H), 7.47 (td, J = 9.0, 2.2 Hz, 1H), 7.40 (d, J = 2.5 Hz, 1H), 7.20 (dd, J = 6.6, 2.6 Hz, 1H), 4.46 (d, J = 10.6 Hz, 1H), 4.25-4.12 (m, 2H), 4.10- 3.85 (m, 5H), 3.51-3.45 (m, 2H), 3.41-3.33 (m, 1H), 2.24-2.15 (m, 4H), 1.91-1.66 (m, 2H), 1.65- 1.49 (m, 7H), 1.49-1.34 (m, 1H), 1.31-1.04 (m, 2H). |
| 1-137 | 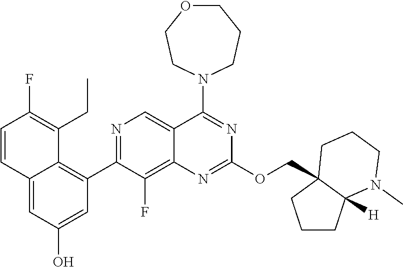 [SMILES: CCc1c(F)ccc2cc(O)cc(-c3ncc4c(N5CCCOCC5)nc(OC[C@]56CCC[C@H]5N(C)CCC6)nc4c3F)c12] | 5-ethyl-6-fluoro-4- (8-fluoro-2- (((4aS,7aR)-1- methyloctahydro- 4aH-cyclopenta[b] pyridin-4a-yl) methoxy)- 4-(1,4-oxazepan- 4-yl)pyrido[4,3- d]pyrimidin-7- yl)naphthalen- 2-ol | 604.3 | ¹H NMR (400 MHz, DMSO-d₆) δ ppm = 9.93 (s, 1H), 9.16 (s, 1H), 7.77 (dd, J = 9.1, 6.1 Hz, 1H), 7.45- 7.29 (m, 2H), 7.02 (d, J = 2.5 Hz, 1H), 4.49 (t, J = 10.1 Hz, 1H), 4.23-4.07 (m, 5H), 3.99-3.92 (m, 2H), 3.81-3.67 (m, 2H), 2.71-2.65 (m, 1H), 2.51- 2.33 (m, 3H), 2.27-2.04 (m, 7H), 1.88-1.70 (m, 1H), 1.67-1.35 (m, 8H), 0.74 (t, J = 7.3 Hz, 3H). |
| 1-138 | 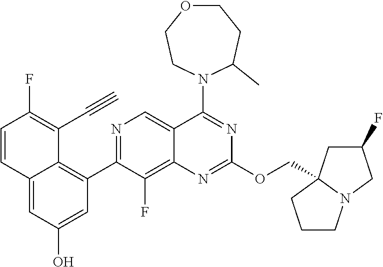 [SMILES: C#Cc1c(F)ccc2cc(O)cc(-c3ncc4c(N5CCOCCC5C)nc(OC[C@@]56CCCN5C[C@H](F)C6)nc4c3F)c12] | 5-ethynyl-6-fluoro- 4-(8-fluoro-2- (((2R,7aS)-2- fluorotetrahydro- 1H-pyrrolizin- 7a(5H)- yl)methoxy)-4-(5- methyl-1,4- oxazepan-4- yl)pyrido[4,3- d]pyrimidin-7- yl)naphthalen- 2-ol | 604.2 | ¹H NMR (400 MHz, DMSO-d₆) δ ppm = 10.15 (s, 1H), 9.05-8.80 (m, 1H), 7.98 (dd, J = 9.3, 6.0 Hz, 1H), 7.53-7.42 (m, 1H), 7.40 (d, J = 2.5 Hz, 1H), 7.25-7.06 (m, 1H), 5.43- 5.17 (m, 1H), 4.88-4.58 (m, 1H), 4.49-4.33 (m, 1H), 4.21-4.03 (m, 3H), 4.02-3.91 (m, 1H), 3.88- 3.55 (m, 3H), 3.47-3.38 (m, 1H), 3.20-2.98 (m, 3H), 2.94-2.78 (m, 1H), 2.38-2.32 (m, 1H), 2.23- 1.70 (m, 7H), 1.45 (t, J = 5.8 Hz, 3H). |
| 1-139^{I} | 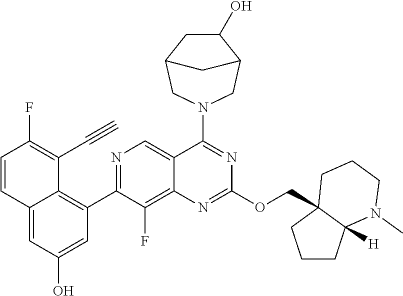 [SMILES: C#Cc1c(F)ccc2cc(O)cc(-c3ncc4c(N5CC6CC(O)C(C6)C5)nc(OC[C@]56CCC[C@H]5N(C)CCC6)nc4c3F)c12] | 3-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-3- azabicyclo[3.2.1 ]octan-6-ol | 626.3 | ¹H NMR (400 MHz, DMSO-d⁶) δ ppm = 10.15 (brs, 1H), δ 9.38-9.04 (m, 1H), 7.98-7.91 (m, 1H), 7.48-7.35 (m, 2H), 7.28- 7.17 (m, 1H), 5.04 (br d, J = 11.3 Hz, 1H), 4.62 (br t, J = 11.9 Hz, 1H), 4.50-4.41 (m, 2H), 4.22- 4.04 (m, 3H), 3.89 (br t, J = 5.6 Hz, 1H), 3.76 (s, 1H), 3.66 (br d, J = 13.3 Hz, 2H), 3.02-2.93 (m, 1H), 2.27-1.99 (m, 4H), 1.85-1.78 (m, 2H), 1.72- 1.52 (m, 7H), 1.43-1.20 (m, 3H), 1.02-0.92 (m, 3H). |
| 1-140 | 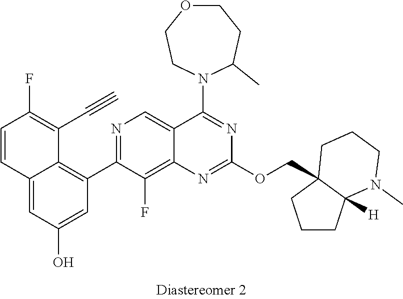 [SMILES: C#Cc1c(F)ccc2cc(O)cc(-c3ncc4c(N5CCOCCC5C)nc(OC[C@]56CCC[C@H]5N(C)CCC6)nc4c3F)c12] | 5-ethynyl-6-fluoro- 4-(8-fluoro-4-(5- methyl-1,4- oxazepan-4-yl)-2- (((4aS,7aR)-1- methyloctahydro- 4aH-cyclopenta[b] pyridin-4a- yl)methoxy)pyrido [4,3-d]pyrimidin- 7-yl)naphthalen-2-ol | 614.3 | ¹H NMR (400 MHz, DMSO-d₆) δ ppm = 10.16 (s, 1H), 9.04-8.79 (m, 1H), 7.98 (dd, J = 9.4, 5.9 Hz, 1H), 7.47 (t, J = 9.1 Hz, 1H), 7.40 (d, J = 2.3 Hz, 1H), 7.24-7.07 (m, 1H), 4.92-4.58 (m, 1H), 4.50- 4.31 (m, 2H), 4.26-4.16 (m, 1H), 4.15-4.06 (m, 1H), 4.04-3.92 (m, 1H), 3.91-3.73 (m, 2H), 3.72- 3.58 (m, 2H), 3.48-3.41 (m, 1H), 2.78-2.67 (m, 1H), 2.39-2.15 (m, 5H), 2.01-1.79 (m, 3H), 1.75- 1.50 (m, 8H), 1.46 (br t, J = 6.1, 3H). |
| 1-141 | 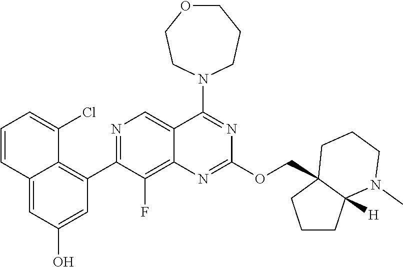 [SMILES: CN1CCC[C@@]2(COc3nc(N4CCCOCC4)c4cnc(-c5cc(O)cc6cccc(Cl)c56)c(F)c4n3)CCC[C@@H]12] | 5-chloro-4-(8- fluoro-2- (((4aS,7aR)-1- methyloctahydro- 4aH-cyclopenta[b] pyridin-4a- yl)methoxy)- 4-(1,4-oxazepan-4- yl)pyrido[4,3- d]pyrimidin-7- yl)naphthalen-2-ol | 592.2 | ¹H NMR (400 MHz, DMSO-d₆) δ ppm = 10.5- 10.01 (brs, 1H), 9.14 (s, 1H), 7.84 (dd, J = 8.1, 1.1 Hz, 1H), 7.44-7.34 (m, 3H), 7.14 (d, J = 2.5 Hz, 1H), 4.46 (dd, J = 10.7, 5.1 Hz, 1H), 4.20-4.12 (m, 5H), 4.04-3.87 (m, 2H), 3.76 (br t, J = 4.7 Hz, 2H), 2.67-2.59 (m, 1H), 2.57- 2.53 (m, 1H), 2.23-2.14 (m, 4H), 2.08 (br d, J = 7.8 Hz, 2H), 1.83-1.79 (m, 1H), 1.73-1.69 (m, 1H), 1.65-1.49 (m, 7H), 1.43- 1.39 (m, 1H). |
| 1-142 | 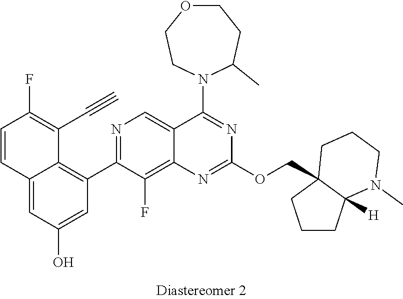 [SMILES: C#Cc1c(F)ccc2cc(O)cc(-c3ncc4c(N5CCOCCC5C)nc(OC[C@]56CCC[C@H]5N(C)CCC6)nc4c3F)c12] | 5-ethynyl-6-fluoro- 4-(8-fluoro-4-(5- methyl-1,4- oxazepan-4-yl)-2- (((4aS,7aR)-1- methyloctahydro- 4aH-cyclopenta[b] pyridin-4a- yl)methoxy)pyrido [4,3-d]pyrimidin- 7-yl)naphthalen- 2-ol | 614.2 | ¹H NMR (400 MHz, DMSO-d₆) δ ppm = 10.21 (s, 1H), 9.03-8.86 (m, 1H), 7.98 (dd, J = 9.0, 6.0 Hz, 1H), 7.47 (t, J = 9.0 Hz, 1H), 7.39 (d, J = 2.3 Hz, 1H), 7.24-7.08 (m, 1H), 4.72 (s, 1H), 4.50-4.31 (m, 2H), 4.19-4.06 (m, 2H), 3.90-3.59 (m, 6H), 2.67- 2.60 (m, 1H), 2.37-2.32 (m, 1H), 2.24-2.12 (m, 4H), 2.02-1.68 (m, 3H), 1.60-1.62 (m, 1H), 1.64- 1.49 (m, 6H), 1.48-1.35 (m, 4H). |
| 1-143^{G} | 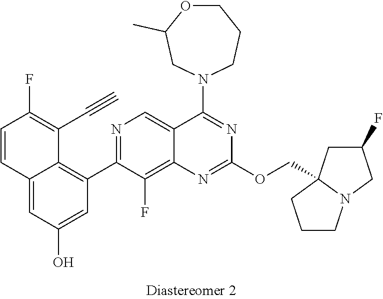 [SMILES: C#Cc1c(F)ccc2cc(O)cc(-c3ncc4c(N5CCCOC(C)C5)nc(OC[C@@]56CCCN5C[C@H](F)C6)nc4c3F)c12] | 5-ethynyl-6-fluoro- 4-(8-fluoro-2- (((2R,7aS)-2- fluorotetrahydro- 1H-pyrrolizin- 7a(5H)- yl)methoxy)-4- (2-methyl-1,4- oxazepan-4- yl)pyrido[4,3- d]pyrimidin-7- yl)naphthalen- 2-ol | 604.2 | ¹H NMR (400 MHz, DMSO-d₆) δ ppm = 10.6- 10.01 (br, 1H), 9.09 (d, J = 13.5 Hz, 1H), 8.01-7.89 (m, 1H), 7.45 (t, J = 8.9 Hz, 1H), 7.38 (s, 1H), 7.19 (dd, J = 10.9, 2.4 Hz, 1H), 5.39- 5.14 (m, 1H), 4.52-4.30 (m, 1H), 4.17-3.86 (m, 7H), 3.71-3.54 (m, 1H), 3.21-3.19 (m, 2H), 2.88- 2.81 (m, 2H), 2.23-1.96 (m, 6H), 1.90-1.74 (m, 3H), 1.28-1.09 (m, 3H). |
| 1-144^{B} | 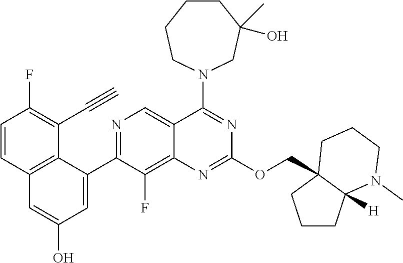 [SMILES: C#Cc1c(F)ccc2cc(O)cc(-c3ncc4c(N5CCCCC(C)(O)C5)nc(OC[C@]56CCC[C@H]5N(C)CCC6)nc4c3F)c12] | 1-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-3- methylazepan- 3-ol | 628.3 | ¹H NMR (400 MHz, DMSO-d₆) δ ppm = 9.43- 9.21 (m, 1H), 8.51 (brs, 1H), 7.97 (dd, J = 8.9, 6.1 Hz, 1H), 7.46 (t, J = 8.9 Hz, 1H), 7.39 (d, J = 2.3 Hz, 1H), 7.19 (dd, J = 12.0, 2.8 Hz, 1H), 4.98-4.78 (m, 1H), 4.49-4.34 (m, 2H), 4.20-4.17 (m, 2H), 4.04- 3.93 (m, 1H), 3.93-3.85 (m, 1H), 3.83-3.72 (m, 2H), 2.66-2.61 (m, 1H), 2.26-2.03 (m, 7H), 1.92- 1.50 (m, 12H), 1.46-1.33 (m, 1H), 1.28-1.22 (m, 3H). |
| 1-145 | 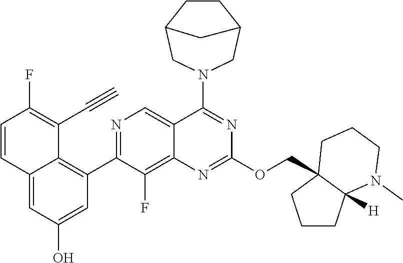 [SMILES: C#Cc1c(F)ccc2cc(O)cc(-c3ncc4c(N5CC6CCC(C6)C5)nc(OC[C@]56CCC[C@H]5N(C)CCC6)nc4c3F)c12] | 4-(4-(3- azabicyclo[3.2.1] octan-3-yl)- 8-fluoro- 2-(((4aS,7aR)-1- methyloctahydro- 4aH-cyclopenta[b] pyridin-4a- yl)methoxy)pyrido [4,3-d]pyrimidin- 7-yl)-5- ethynyl-6- fluoronaphthalen- 2-ol | 610.3 | ¹H NMR (400 MHz, DMSO-d₆) δ ppm = 9.16 (d, J = 9.5 Hz, 1H), 8.53-8.43 (brs, 1H), 7.95 (dd, J = 9.5, 6.0 Hz, 1H), 7.45 (t, J = 9.0 Hz, 1H), 7.38 (s, 1H), 7.19 (dd, J = 14.8, 2.5 Hz, 1H), 4.54-4.30 (m, 2H), 4.23- 4.04 (m, 2H), 4.01-3.91 (m, 2H), 2.67-2.61 (m, 1H), 2.47-2.40 (m, 1H), 2.24-2.03 (m, 5H), 1.92- 1.81 (m, 2H), 1.79-1.74 (brs, 8H), 1.65-1.47 (m, 6H), 1.46-1.27 (m, 2H). |
| 1-146^{B} | 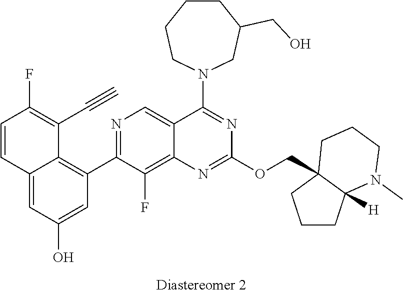 [SMILES: C#Cc1c(F)ccc2cc(O)cc(-c3ncc4c(N5CCCCC(CO)C5)nc(OC[C@]56CCC[C@H]5N(C)CCC6)nc4c3F)c12] | 5-ethynyl-6-fluoro- 4-(8-fluoro-4-(3- (hydroxymethyl) azepan-1-yl)-2- (((4aS,7aR)-1- methyloctahydro- 4aH-cyclopenta[b] pyridin-4a- yl)methoxy)pyrido [4,3-d]pyrimidin- 7-yl)naphthalen- 2-ol | 628.3 | ¹H NMR (400 MHz, DMSO-d₆) δ ppm = 10.6- 10.01 (br, 1H), 9.04 (s, 1H), 7.95 (dd, J = 9.3, 6.0 Hz, 1H), 7.45 (t, J = 9.0 Hz, 1H), 7.38 (d, J = 2.5 Hz, 1H), 7.19 (d, J = 2.5 Hz, 1H), 4.62-4.51 (m, 1H), 4.49-4.36 (m, 2H), 4.13 (d, J = 10.8 Hz, 1H), 3.95- 3.88 (m, 1H), 3.62-3.49 (m, 3H), 2.68-2.62 (m, 1H), 2.48-2.35 (m, 2H), 2.25-2.19 (m, 1H), 2.17 (s, 3H), 1.87-1.76 (m, 2H), 1.73-1.47 (m, 15H), 1.43- 1.32 (m, 1H). |
| 1-147 | 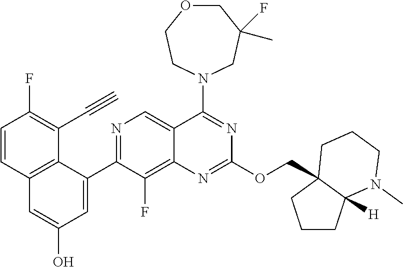 [SMILES: C#Cc1c(F)ccc2cc(O)cc(-c3ncc4c(N5CCOCC(C)(F)C5)nc(OC[C@]56CCC[C@H]5N(C)CCC6)nc4c3F)c12] | 5-ethynyl-6-fluoro- 4-(8-fluoro-4-(6- fluoro-6-methyl- 1,4-oxazepan-4- yl)-2-(((4aS, 7aR)-1- methyloctahydro- 4aH-cyclopenta[b] pyridin-4a- yl)methoxy)pyrido [4,3-d]pyrimidin- 7-yl)naphthalen- 2-ol | 632.2 | ¹H NMR (400 MHz, DMSO-d₆) δ ppm = 10.17 (s, 1H), 9.16 (d, J = 8.0 Hz, 1H), 7.98 (dd, J = 9.1, 5.9 Hz, 1H), 7.47 (t, J = 9.0 Hz, 1H), 7.40 (d, J = 2.5 Hz, 1H), 7.20 (dd, J = 6.0, 2.5 Hz, 1H), 4.52-4.11 (m, 6H), 4.06-3.74 (m, 6H), 2.80-2.75 (m, 1H), 2.24- 2.23 (m, 1H), 2.23 (br s, 3H), 1.89-1.49 (m, 10H), 1.47-1.32 (m, 3H). |
| 1-148 | 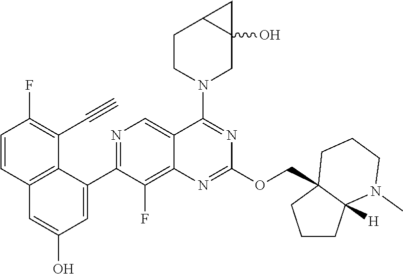 [SMILES: C#Cc1c(F)ccc2cc(O)cc(-c3ncc4c(N5CCC6CC6(O)C5)nc(OC[C@]56CCC[C@H]5N(C)CCC6)nc4c3F)c12] | 3-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-3- azabicyclo[4.1.0] heptan-1-ol | 612.3 | ¹H NMR (400 MHz, DMSO-d₆) δ ppm = 10.18 (br s, 1H), 9.09-8.96 (m, 1H), 7.98 (dd, J = 9.1, 5.9 Hz, 1H), 7.54-7.43 (m, 1H), 7.40 (d, J = 2.5 Hz, 1H), 7.20 (dd, J = 4.6, 2.4 Hz, 1H), 5.81 (br s, 1H), 4.57-4.29 (m, 2H), 4.20- 4.11 (m, 1H), 4.10-3.80 (m, 2H), 3.64-3.51 (m, 2H), 2.70-2.64 (m, 1H), 2.31-2.16 (m, 2H), 2.16 (s, 3H), 1.88-1.70 (m, 3H), 1.66-1.49 (m, 7H), 1.42- 1.26 (m, 3H), 0.94-0.81 (m, 1H), 0.70-0.57 (m, |
| | | | | 1H). (Diastereomeric |
| | | | | mixture) |
| 1-149 | 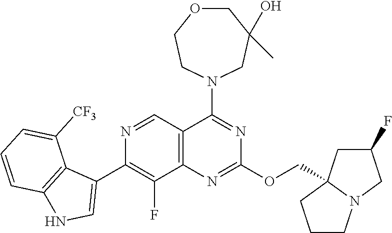 [SMILES: CC1(O)COCCN(c2nc(OC[C@@]34CCCN3C[C@H](F)C4)nc3c(F)c(-c4c[nH]c5cccc(C(F)(F)F)c45)ncc23)C1] | 4-(8-fluoro-2- (((2R,7aS)-2- fluorotetrahydro- 1H-pyrrolizin- 7a(5H)-yl) methoxy)-7-(4- (trifluoromethyl)- 1H-indol-3- yl)pyrido[4,3- d]pyrimidin-4-yl)- 6-methyl-1,4- oxazepan-6-ol | 619.2 | ¹H NMR (400 MHz, DMSO-d₆) δ ppm = 12.08 (d, J = 2.0 Hz, 1H), 9.40 (s, 1H), 7.82 (d, J = 8.3 Hz, 1H), 7.78 (d, J = 2.8 Hz, 1H), 7.46 (d, J = 7.3 Hz, 1H), 7.40-7.29 (m, 1H), 5.39-5.21 (m, 1H), 5.17 (s, 1H), 4.33-4.25 (m, 1H), 4.22-4.11(m, 2H), 4.09-3.93 (m, 4H), 3.90- 3.81 (m, 1H), 3.56 (s, 2H), 3.14-3.00 (m, 3H), 2.87- 2.78 (m, 2H), 2.20-1.97 |
| | | | | (m, 3H), 1.89-1.72 (m, |
| | | | | 3H), 1.15 (s, 3H). |
| 1-150^{C} | 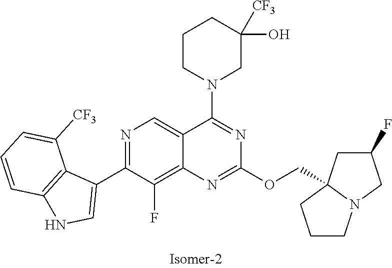 [SMILES: OC1(C(F)(F)F)CCCN(c2nc(OC[C@@]34CCCN3C[C@H](F)C4)nc3c(F)c(-c4c[nH]c5cccc(C(F)(F)F)c45)ncc23)C1] | 1-(8-fluoro-2- (((2R,7aS)-2- fluorotetrahydro- 1H-pyrrolizin- 7a(5H)- yl)methoxy)-7-(4- (trifluoromethyl)- 1H-indol-3- yl)pyrido[4,3- d]pyrimidin- 4-yl)-3- (trifluoromethyl) piperidin-3-ol | 657.2 | ¹H NMR (400 MHz, DMSO-d₆) δ ppm = 12.10 (br d, J = 1.8 Hz, 1H), 9.13 (s, 1H), 7.91-7.72 (m, 2H), 7.47 (d, J = 7.8 Hz, 1H), 7.35 (t, J = 7.8 Hz, 1H), 6.35 (s, 1H), 5.42-5.08 (m, 1H), 4.54-4.45 (m, 1H), 4.39 (br d, J = 12.5 Hz, 1H), 4.18-4.01 (m, 2H), 3.70 (d, J = 13.0 Hz, 1H), 3.14-3.06 (m, 3H), 3.04- 3.01 (m, 1H), 2.89-2.77 (m, 1H), 2.19-1.74 (m, 10H). |
| 1-151^{C} | 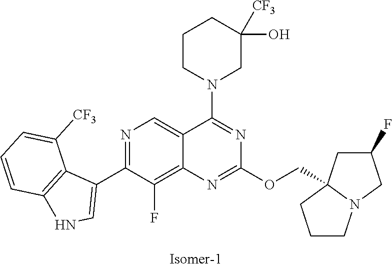 [SMILES: OC1(C(F)(F)F)CCCN(c2nc(OC[C@@]34CCCN3C[C@H](F)C4)nc3c(F)c(-c4c[nH]c5cccc(C(F)(F)F)c45)ncc23)C1] | 1-(8-fluoro-2- (((2R,7aS)-2- fluorotetrahydro- 1H-pyrrolizin- 7a(5H)- yl)methoxy)-7-(4- (trifluoromethyl)- 1H-indol-3- yl)pyrido[4,3- d]pyrimidin- 4-yl)-3- (trifluoromethyl) piperidin-3-ol | 657.2 | ¹H NMR (400 MHz, DMSO-d₆) δ ppm = 12.10 (d, J = 2.3 Hz, 1H), 9.14 (s, 1H), 7.89-7.78 (m, 2H), 7.47 (d, J = 7.5 Hz, 1H), 7.40-7.28 (m, 1H), 6.35 (s, 1H), 5.44-5.16 (m, 1H), 4.49 (br d, J = 13.5 Hz, 1H), 4.40 (br d, J = 13.5 Hz, 1H), 4.22-4.02 (m, 2H), 3.71 (d, J = 13.3 Hz, 1H), 3.20-2.98 (m, 4H), 2.92-2.79 (m, 1H), 2.20- 1.73 (m, 10H). |
| 1-152 | 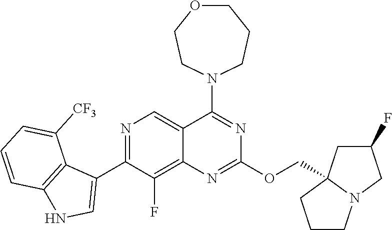 [SMILES: Fc1c(-c2c[nH]c3cccc(C(F)(F)F)c23)ncc2c(N3CCCOCC3)nc(OC[C@@]34CCCN3C[C@H](F)C4)nc12] | 4-(8-fluoro-2- (((2R,7aS)-2- fluorotetrahydro- 1H-pyrrolizin- 7a(5H)- yl)methoxy)-7-(4- (trifluoromethyl)- 1H-indol-3- yl)pyrido[4,3- d]pyrimidin-4-yl)- 1,4-oxazepane | 589.8 | ¹H NMR (400 MHz, DMSO-d₆) δ ppm = 12.09 (d, J = 2.5 Hz, 1H), 9.12 (s, 1H), 7.83 (d, J = 8.3 Hz, 1H), 7.78 (d, J = 2.8 Hz, 1H), 7.46 (d, J = 7.3 Hz, 1H), 7.34 (t, J = 8.0 Hz, 1H), 5.44-5.14 (m, 1H), 4.22-4.02 (m, 6H), 3.99- 3.91 (m, 2H), 3.81-3.68 (m, 2H), 3.21-2.97 (m, 3H), 2.92-2.78 (m, 1H), 2.21-1.98 (m, 5H), 1.92- 1.72 (m, 3H). |
| 1-153^{B} | 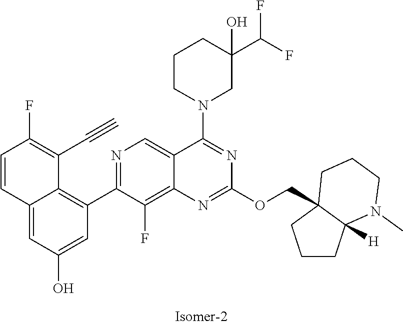 [SMILES: C#Cc1c(F)ccc2cc(O)cc(-c3ncc4c(N5CCCC(O)(C(F)F)C5)nc(OC[C@]56CCC[C@H]5N(C)CCC6)nc4c3F)c12] | 3-(difluoromethyl)- 1-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)piperidin- 3-ol | 650.2 | ¹H NMR (400 MHz, DMSO-d₆) δ ppm = 10.18 (br s, 1H), 9.27-8.99 (m, 1H), 7.98 (dd, J = 9.3, 5.8 Hz, 1H), 7.47 (dt, J = 9.1, 1.1 Hz, 1H), 7.40 (d, J = 2.5 Hz, 1H), 7.22 (dd, J = 19.3, 2.5 Hz, 1H), 5.67 (d, J = 14.5 Hz, 1H), 4.53-4.42 (m, 2H), 4.31 (brd, J= 13.5 Hz, 1H), 4.16 (d, J = 10.8 Hz, 1H), 4.01-3.82 (m,1H), 3.71-3.59 (m, 1H), 2.70-2.64 (m, 2H), 2.49-2.43 (m, 1H), 2.29- 2.22 (m, 2H), 2.19 (s, 3H), 1.89-1.70 (m, 5H), 1.67- 1.50 (m, 9H). |
| 1-154^{B} | 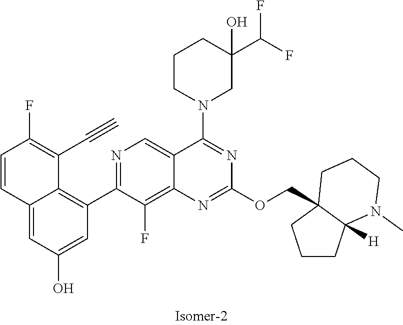 [SMILES: C#Cc1c(F)ccc2cc(O)cc(-c3ncc4c(N5CCCC(O)(C(F)F)C5)nc(OC[C@]56CCC[C@H]5N(C)CCC6)nc4c3F)c12] | 3-(difluoromethyl)- 1-(7-(8-ethynyl-7- fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)piperidin- 3-ol | 650.2 | ¹H NMR (400 MHz, DMSO-d₆) δ ppm = 10.18 (br dd, J = 3.8, 3.0 Hz, 1H), 9.28-8.89 (m, 1H), 7.98 (dd, J = 9.3, 6.0 Hz, 1H), 7.46 (t, J = 9.8 Hz, 1H), 7.42-7.38 (m, 1H), 7.22 (dd, J = 19.6, 2.4 Hz, 1H), 6.16-5.76 (m, 1H), 5.67 (d, J = 14.8 Hz, 1H), 4.57- 4.42 (m, 2H), 4.36-4.18 (m, 1H), 4.14 (dd, J = 11.0, 4.8 Hz, 1H), 3.97-3.84 (m, 1H), 3.65 (t, J = 13.0 Hz, 1H), 3.54-3.50 (m, 1H), 2.74-2.66 (m,1H), 2.31- 2.23 (m, 2H), 2.20 (s, 3H), 1.89-1.70 (m, 5H), 1.68- 1.48 (m, 9H). |
| 1-155 | 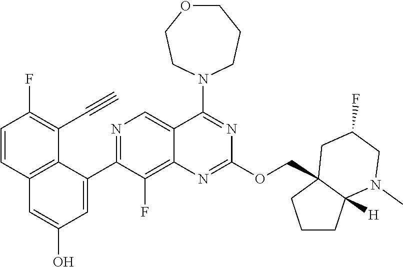 [SMILES: C#Cc1c(F)ccc2cc(O)cc(-c3ncc4c(N5CCCOCC5)nc(OC[C@]56CCC[C@H]5N(C)C[C@@H](F)C6)nc4c3F)c12] | 5-ethynyl-6-fluoro- 4-(8-fluoro-2- (((35,4aS,7aR)-3- fluoro-1- methyloctahydro- 4aH-cyclopenta[b] pyridin-4a- yl)methoxy)- 4-(1,4-oxazepan-4- yl)pyrido[4,3- d]pyrimidin-7- yl)naphthalen-2-ol | 618.3 | ¹H NMR (400 MHz, DMSO-d₆) δ ppm = 10.16 (br s, 1H), 9.10 (d, J = 1.5 Hz, 1H), 7.98 (dd, J = 9.1, 5.6 Hz, 1H), 7.47 (t, J = 9.0 Hz, 1H), 7.40 (d, J = 2.5 Hz, 1H), 7.18 (d, J = 2.5 Hz, 1H), 4.97-4.65 (m, 1H), 4.41 (t, J = 10.9 Hz, 1H), 4.21-4.08 (m, 5H), 4.00 (dd, J = 3.5, 1.0 Hz, 1H), 3.95 (t, J = 4.6 Hz, 2H), 3.82-3.66 (m, 2H), 2.74-2.58 (m, 2H), 2.24 (d, J = 1.8 Hz, 3H), 2.15-1.96 (m, 3H), 1.89-1.47 (m, 8H). |
| 1-156 | 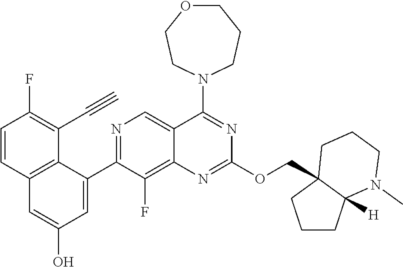 [SMILES: C#Cc1c(F)ccc2cc(O)cc(-c3ncc4c(N5CCCOCC5)nc(OC[C@]56CCC[C@H]5N(C)CCC6)nc4c3F)c12] | 5-ethynyl-6-fluoro- 4-(8-fluoro-2- (((4aS,7aR)-1- methyloctahydro- 4aH-cyclopenta[b] pyridin-4a- yl)methoxy)- 4-(1,4-oxazocan- 4-yl)pyrido[4,3- d]pyrimidin-7- yl)naphthalen- 2-ol | 614.3 | ¹H NMR (400 MHz, DMSO-d⁶) δ ppm = 10.16 (s, 1H), 9.02 (s, 1H), 7.98 (dd, J = 9.1, 6.1 Hz, 1H), 7.47 (t, J = 9.0 Hz, 1H), 7.40 (d, J = 2.5 Hz, 1H), 7.20 (d, J = 2.3 Hz, 1H), 4.43 (d, J = 10.8 Hz, 1H), 4.37-4.27 (m, 1H), 4.17 (dd, J = 10.5, 3.8 Hz, 1H), 4.13-3.90 (m, 5H), 3.73- 3.65 (m, 2H), 3.09 (br dd, J = 3.6, 1.6 Hz, 1H), 2.68 (br d, J = 1.5 Hz, 1H), 2.29 -2.14 (m, 4H), 2.09- 2.04 (m, 1H), 1.84-1.77 |
| | | | | (m, 2H), 1.70-1.46 (m, |
| | | | | 11H), 1.45-1.38 (m, 1H). |
| 1-157 | 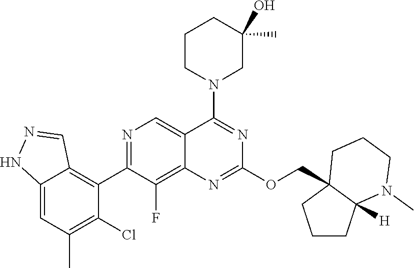 [SMILES: Cc1cc2[nH]ncc2c(-c2ncc3c(N4CCC[C@@](C)(O)C4)nc(OC[C@]45CCC[C@H]4N(C)CCC5)nc3c2F)c1Cl] | (3R)-1-(7-(5- chloro-6-methyl- 1H-indazol-4-yl)- 8-fluoro-2-(((4aS)- 1-methyloctahydro- 4aH-cyclopenta[b] pyridin-4a- yl)methoxy)pyrido [4,3-d]pyrimidin- 4-yl)-3- methylpiperidin- 3-ol | 594.2 | ¹H NMR (400 MHz, DMSO-d⁶) δ ppm = 13.28 (s, 1H), 9.38-9.22 (m, 1H), 7.70 (d, J = 5.5 Hz, 2H), 4.87-4.67 (m, 1H), 4.56- 4.47 (m, 1H), 4.42-4.39 (m, 1H), 4.21-4.13 (m, 1H), 4.12-4.00 (m, 1H), 3.62-3.54 (m, 2H), 2.73- 2.69 (m, 1H), 2.38-2.20 (m, 2H), 2.21 (br s, 3H), 1.99 (s, 3H), 1.89-1.82 (m, 1H), 1.78-1.47 (m, 12H), 1.45-1.37 (m, 1H), 1.20 (d, J = 1.0 Hz, 3H). |
| 1-158 | 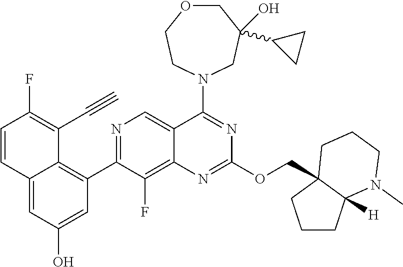 [SMILES: C#Cc1c(F)ccc2cc(O)cc(-c3ncc4c(N5CCOCC(O)(C6CC6)C5)nc(OC[C@]56CCC[C@H]5N(C)CCC6)nc4c3F)c12] | 6-cyclopropyl-4-(7- (8-ethynyl- 7-fluoro-3- hydroxynaphthalen- 1-yl)-8-fluoro-2- (((4aS,7aR)-1- methyloctahydro- 4aH-cyclopenta[b] pyridin-4a- yl)methoxy)pyrido [4,3-d]pyrimidin- 4-yl)-1,4-oxazepan- 6-ol | 656.3 | ¹H NMR (400 MHz, DMSO-d⁶) δ ppm = 10.19 (br s, 1H), 9.52-9.20 (m, 1H), 7.98 (dd, J = 9.1, 5.9 Hz, 1H), 7.47 (t, J = 9.0 Hz, 1H), 7.40 (d, J = 2.5 Hz, 1H), 7.20 (dd, J = 8.0, 2.5 Hz, 1H), 5.01- 4.65 (m, 1H), 4.52-4.28 (m, 3H), 4.24-4.08 (m, 3H), 4.03-3.85 (m, 3H), 3.78-3.72 (m, 1H), 3.59 (br d, J = 7.5 Hz, 1H), 2.92 (br s, 1H), 2.71-2.63 (m, 1H), 2.33 (br s, 3H), 1.95-1.42 (m, 11H), 0.97- 0.78 (m, 1H), 0.52-0.36 |
| | | | | (m, 2H), 0.33- 0.19 |
| | | | | (m, 2H). |
| | | | | (Diastereomeric mixture) |

### Preparation of Intermediate 116: tert-butyl 3-(2-{[(4aS,7aR)-1-methyl-octahydro-1H-cyclopenta[b]pyridin-4a-yl]methoxy}-7-chloro-8-fluoropyrido[4,3-d]pyrimidin-4yl)-3,8-diazabicyclo[3.2.1]octane-8-carboxylate

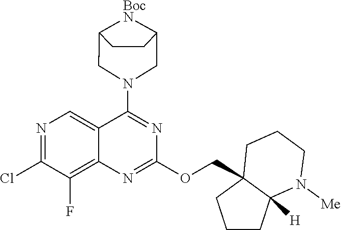 [SMILES: CN1CCC[C@@]2(COc3nc(N4CC5CCC(C4)N5C(=O)OC(C)(C)C)c4cnc(Cl)c(F)c4n3)CCC[C@@H]12]

To a stirred solution tert-butyl 3{2,7-dichloro-8-fluoropyrido[4,3-d]pyrimidin-4-yl}-3,7-diazabicyclo[3.2.1]octane-8carboxylate (5.0 g, 11.67 mmol) and ((4aS,7aR)-2-methyloctahydro-4aH-cyclopenta[b]pyridin-4a-yl)methanol (2.17 g, 12.84 mmol) in THF (60 mL) was added 1M LiHMDS (lithium hexamethyldisilazide) in hexanes (35.0 mL, 35.0 mmol) at 0° C. under nitrogen. The reaction mixture was allowed to warm to room temperature and stirred for 15 h. The reaction mixture was quenched with saturated ammonium chloride solution (20 mL) and extracted with EtOAc. The combined organic extracts were washed with water and brine solution, dried over Na₂SO₄, filtered, and evaporated under reduced pressure to provide a crude residue, which was purified by silica gel column chromatography using COMBIFLASH™ chromatography (Teledyne ISO, Lincoln, NE) (120 g REDISEP™ column (Teledyne ISCO, Lindcoln, NE), 80 to 100% EtOAc—pet ether) to afford tert-butyl 3-(2-{[(4aS,7aR)-1-methyl-octahydro-1H-cyclopenta[b]pyridin-4a-yl]methoxy}-7-chloro-8-fluoropyrido[4,3-d]pyrimidin-4yl)-3,8-diazabicyclo[3.2.1]octane-8-carboxylate (3 g, 5.35 mmol, 46% yield) as a pale brown solid. MS(ESI) m/z: 562.3 (M+H)⁺.

### Preparation of Intermediate 117: tert-butyl 3(2-{[(4aS,7aR)-1-methyl-octahydro-1H-cyclopenta[b]pyridin-4a-yl]methoxy}-8-fluoro-3-(methoxymethoxy)-8{2-[tris(propan-2-yl)silyl]ethynyl}naphthalen-1yl]pyrido[4,3-d]pyrimidin-4yl)-3,8-diazabicyclo[3.2.1]octane-8-carboxylate

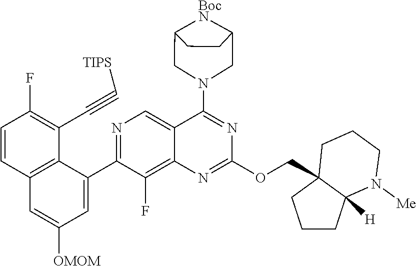

To a stirred solution of tert-butyl-3-(2-{[(4aS,7aR)-1-methyl-octahydro-1H-cyclopenta[b]pyridin-4a-yl]methoxy}-7-chloro-8-fluoropyrido[4,3-d]pyrimidin-4-yl)-3,8-diazabicyclo[3.2.1]octane-8-carboxylate (0.9 g, 1.60 mmol) in 1,4-dioxane (9 mL) and water (3 mL) was added ((2-fluoro-6-(methoxymethoxy)-8-(4,4,5,5-tetramethyl-1,3,2-dioxaborolan-2-yl)naphthalen-1-yl)ethynyl)triisopropylsilane (0.91 g, 1.76 mmol), and Cs₂CO₃ (1.57 g, 4.81 mmol) followed by PdCl₂(dppf) (dppf: 1,1′-bis(diphenylphosphino)ferrocene) (0.12 g, 0.16 mmol). The reaction mixture was purged with N2 for 5 min and heated at 105° C. for 1 h in a microwave reactor. The reaction mixture was cooled to room temperature, diluted with water, and extracted with EtOAc. The organic layer was washed with brine, dried over anhydrous sodium sulfate, filtered, and concentrated under reduced pressure to afford the crude product, which was purified by COMBIFLASH™ chromatography (Teledyne ISO, Lincoln, NE) (using 40 g silica gel column, using 50 to 100% EtOAc/Pet ether) to afford tert-butyl 3-(2-{[(4aS,7aR)-1-methyl-octahydro-1H-cyclopenta[b]pyridin-4a-yl]methoxy}-8-fluoro-7-[7-fluoro-3-(methoxymethoxy)-8-{2-[tris(propan-2-yl)silyl]ethynyl}naphthalen-1-yl]pyrido[4,3-d]pyrimidin-4-yl)-3,8-diazabicyclo[3.2.1]octane-8-carboxylate (0.8 g, 0.88 mmol, 55% yield) as a brown solid. MS(ESI) m/z: 911.5 [M+H]⁺.

### Preparation of Intermediate 118: tert-butyl 3-(2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoropyrido[4,3-d]pyrimidin-4-yl)-3,8-diazabicyclo[3.2.1]octane-8-carboxylate

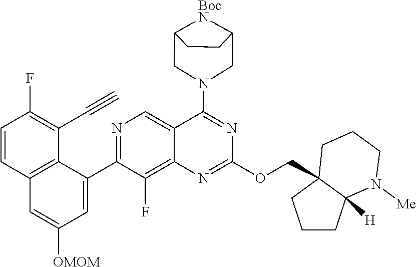

To a stirred solution of tert-butyl 3-(2-{[(4aS,7aR)-1-methyl-octahydro-1H-cyclopenta[b]pyridin-4a-yl]methoxy}-8-fluoro-7-[7-fluoro-3-(methoxymethoxy)-8-{2-[tris(propan-2-yl)silyl]ethynyl}naphthalen-1-yl]pyrido[4,3-d]pyrimidin-4-yl)-3,8-diazabicyclo[3.2.1]octane-8-carboxylate (0.9 g, 1.0 mmol) in DMF (10 mL) was added CsF (1.5 g, 9.88 mmol). The reaction mixture was heated at 50° C. for 1 h. The reaction mixture was cooled to room temperature, diluted with water, and extracted with EtOAc. The organic layer was washed with brine, dried over anhydrous sodium sulfate, filtered, and concentrated under reduced pressure to afford tert-butyl 3-(2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoropyrido[4,3-d]pyrimidin-4-yl)-3,8-diazabicyclo[3.2.1]octane-8-carboxylate (0.7 g, 0.93 mmol, 94% yield) as a brown oil, which was taken for the next step without further purification. MS(ESI) m/z: 755.3 [M+H]⁺.

### Preparation of Intermediate 119: 2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoropyrido[4,3-d]pyrimidin-4-ol

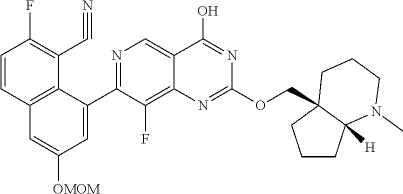

To a stirred solution of 2-{[(4aS,7aR)-1-methyl-octahydro-1H-cyclopenta[b]pyridin-4a-yl]methoxy}-8-fluoro-7-[7-fluoro-3-(methoxymethoxy)-8-{2-[tris(propan-2-yl)silyl]ethynyl}naphthalen-1-yl]pyrido[4,3-d]pyrimidin-4-ol (3.5 g, 4.88 mmol) in DMF (40 mL) was added CsF (7.42 g, 48.8 mmol). The reaction mixture was stirred at 50° C. for 1 h. The reaction mixture was quenched with water and extracted with EtOAc. The combined layer was washed with brine, dried over anhydrous sodium sulfate, and evaporated under reduced pressure to give the crude product. The crude compound was triturated with diethyl ether to get 2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoropyrido[4,3-d]pyrimidin-4-ol (1.8 g, 3.21 mmol, 65.8% yield) as a pale-brown solid. MS(ESI) m/z: 561.2 [M+H]⁺; ¹H NMR (400 MHz, DMSO-d₆, D₂O EXCHANGE) δ ppm=8.86 (d, J=7.0 Hz, 1H), 8.10-8.02 (m, 1H), 7.68 (dd, J=8.5, 2.5 Hz, 1H), 7.51 (td, J=9.0, 3.5 Hz, 1H), 7.30 (d, J=3.0 Hz, 1H), 5.38-5.26 (m, 2H), 4.36-4.23 (m, 2H), 3.93 (d, J=13.0 Hz, 1H), 3.44-3.36 (m, 3H), 3.19-3.02 (m, 1H), 2.86-2.68 (m, 2H), 2.43 (br d, J=10.5 Hz, 3H), 2.01-1.43 (m, 10H).

### Preparation of Intermediate 120: 2-{[(4aS,7aR)-1-methyl-octahydro-1H-cyclopenta[b]pyridin-4a-yl]methoxy}-7-[8-ethyl-7-fluoro-3-(methoxymethoxy)naphthalen-1-yl]-8-fluoropyrido[4,3-d]pyrimidin-4-ol

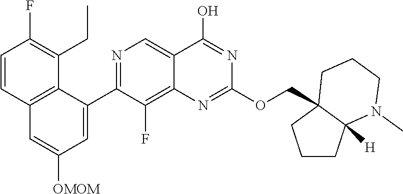

To a solution of 2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoropyrido[4,3-d]pyrimidin-4-ol (10.0 g, 17.84 mmol) in 1:2 MeOH-THF (300 mL) was added palladium on carbon (50% wet; 10.44 g, 9.81 mmol), and the reaction mixture was stirred under hydrogen bladder over a period of 4 hours. The reaction mixture was filtered through a diatomaceous earth pad (CELITE™, Sigma Aldrich, St. Louis, MO), washed with MeOH (500 mL) and the filtrate was evaporated to get a crude residue, which was triturated with pet ether to afford 2-{[(4aS,7aR)-1-methyl-octahydro-1H-cyclopenta[b]pyridin-4a-yl]methoxy}-7-[8-ethyl-7-fluoro-3-(methoxymethoxy)naphthalen-1-yl]-8-fluoropyrido[4,3-d]pyrimidin-4-ol (8.6 g, 15.23 mmol, 85% yield) as a pale brown solid. MS(ESI) m/z: 565.3 (M+H)*.

### Preparation of Intermediate 121: 4-(2-{[(4aS,7aR)-1-methyl-octahydro-1H-cyclopenta[b]pyridin-4a-yl]methoxy}-7-[8-ethyl-7-fluoro-3-(methoxymethoxy)naphthalen-1-yl]-8-fluoropyrido[4,3-d]pyrimidin-4-yl)-6-methyl-1,4-oxazepan-6-ol

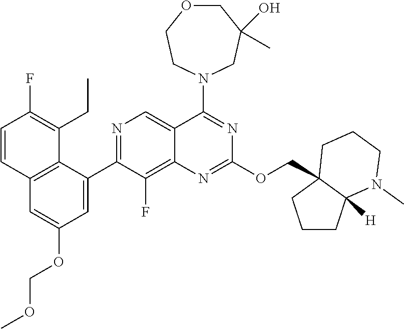 [SMILES: CCc1c(F)ccc2cc(OCOC)cc(-c3ncc4c(N5CCOCC(C)(O)C5)nc(OC[C@]56CCC[C@H]5N(C)CCC6)nc4c3F)c12]

To a stirred suspension of 2-{[(4aS,7aR)-1-methyl-octahydro-1H-cyclopenta[b]pyridin-4a-yl]methoxy}-7-[8-ethyl-7-fluoro-3-(methoxymethoxy)naphthalen-1-yl]-8-fluoropyrido[4,3-d]pyrimidin-4-ol (8.6 g, 15.23 mmol) in acetonitrile (120 mL) at room temperature, were added 6-methyl-1,4-oxazepan-6-ol hydrochloride [Intermediate 75); 2.81 g, 16.75 mmol], PyBOP (11.89 g, 22.85 mmol) and DIPEA (7.98 mL, 45.7 mmol). The reaction mixture was heated at 80° C. for 6 hours. Then, the reaction mixture was then cooled to room temperature, concentrated under reduced pressure to get a crude residue, which was diluted with ethyl acetate. The organic layer was washed with water, brine, dried over anhydrous sodium sulfate, filtered and concentrated under reduced pressure to get a crude residue, which was purified by silicagel column chromatography using COMBIFLASH™ instrument (120 g REDISEP™ column (Teledyne ISCO, Lincoln, NE), 60-100% EtOAc in Petroleum ether) to afford 4-(2-{[(4aS,7aR)-1-methyl-octahydro-1H-cyclopenta[b]pyridin-4a-yl]methoxy}-7-[8-ethyl-7-fluoro-3-(methoxymethoxy)naphthalen-1-yl]-8-fluoropyrido[4,3-d]pyrimidin-4-yl)-6-methyl-1,4-oxazepan-6-ol (6.2 g, 9.15 mmol, 60.1% yield) as a brown solid. MS(ESI) m/z: 678.3 [M+H]⁺; ¹H NMR (400 MHz, DMSO-d₆) δ ppm=9.55 (br s, 1H), 7.91 (dd, J=8.9, 6.1 Hz, 1H), 7.68 (d, J=1.9 Hz, 1H), 7.44 (t, J=9.4 Hz, 1H), 7.22 (br d, J=12.4 Hz, 1H), 5.40 (s, 2H), 5.24-5.12 (m, 1H), 4.45-4.18 (m, 3H), 4.15-3.95 (m, 3H), 3.91-3.68 (m, 1H), 3.64-3.51 (m, 2H), 3.48 (s, 3H), 3.20-3.11 (m, 2H), 2.87-2.71 (m, 3H), 2.26-2.09 (m, 2H), 1.99 (s, 3H), 1.88-1.44 (m, 8H), 1.20-1.12 (m, 4H), 0.75 (br t, J=6.3 Hz, 3H).

### Preparation of Example 1-134: 4-(2-{[(4aS,7aR)-1-methyl-octahydro-1H-cyclopenta[b]pyridin-4a-yl]methoxy}-7-(8-ethyl-7-fluoro-3-hydroxynaphthalen-1-yl)-8-fluoropyrido[4,3-d]pyrimidin-4-yl)-6-methyl-1,4-oxazepan-6-ol

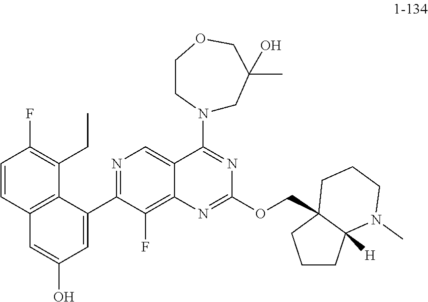 [SMILES: CCc1c(F)ccc2cc(O)cc(-c3ncc4c(N5CCOCC(C)(O)C5)nc(OC[C@]56CCC[C@H]5N(C)CCC6)nc4c3F)c12]

To a stirred solution of 4-(2-{[(4aS,7aR)-1-methyl-octahydro-1H-cyclopenta[b]pyridin-4a-yl]methoxy}-7-[8-ethyl-7-fluoro-3-(methoxymethoxy)naphthalen-1-yl]-8-fluoropyrido[4,3-d]pyrimidin-4-yl)-6-methyl-1,4-oxazepan-6-ol (4.5 g, 6.64 mmol) in EtOAc (90 ml) at 0° C., was added 1M HCl in EtOAc (100 ml, 100 mmol). The reaction mixture was stirred for 1 h. After completion, the reaction mixture was concentrated under reduced pressure to get a crude residue, which was dissolved in DCM (200 mL) and neutralized with TEA. The volatiles were removed under reduced pressure, dissolved in DCM and washed with saturated aqueous NaHCO₃solution. The organic layer was dried over anhydrous sodium sulfate, filtered, and concentrated under reduced pressure to get a crude residue, which was purified by silica gel column chromatography using COMBIFLASH™ instrument (80 g REDISEP™ column (Teledyne ISCO, Lincoln, NE), 4 to 7% MeOH in DCM) to afford 4-(2-{[(4aS,7aR)-1-methyl-octahydro-1H-cyclopenta[b]pyridin-4a-yl]methoxy}-7-(8-ethyl-7-fluoro-3-hydroxynaphthalen-1-yl)-8-fluoropyrido[4,3-d]pyrimidin-4-yl)-6-methyl-1,4-oxazepan-6-ol (3.5 g, 5.43 mmol, 82% yield) as an off white solid. MS(ESI) m/z: 634.3 [M+H]; ¹H NMR (400 MHz, DMSO-d₆) δ ppm=9.96 (br s, 1H), 9.48 (d, J=1.5 Hz, 1H), 7.77 (dd, J=9.0, 6.0 Hz, 1H), 7.42-7.25 (m, 2H), 7.03 (dd, J=14.1, 2.6 Hz, 1H), 5.17 (d, J=11.0 Hz, 1H), 4.54-4.47 (m, 1H), 4.41-4.15 (m, 3H), 4.13-3.82 (m, 5H), 3.63-3.50 (m, 3H), 2.71-2.60 (m, 1H), 2.45 (br d, J=0.8 Hz, 1H), 2.25-2.07 (m, 4H), 1.89-1.68 (m, 2H), 1.66-1.46 (m, 7H), 1.44-1.37 (m, 1H), 1.16 (d, J=5.0 Hz, 3H), 0.74 (dt, J=7.3, 3.3 Hz, 3H).

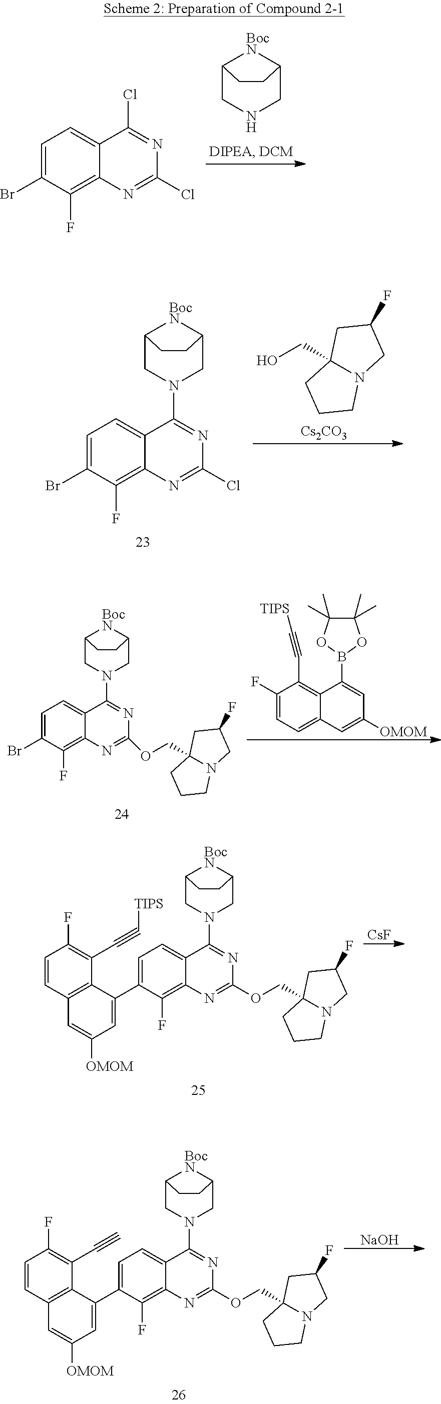

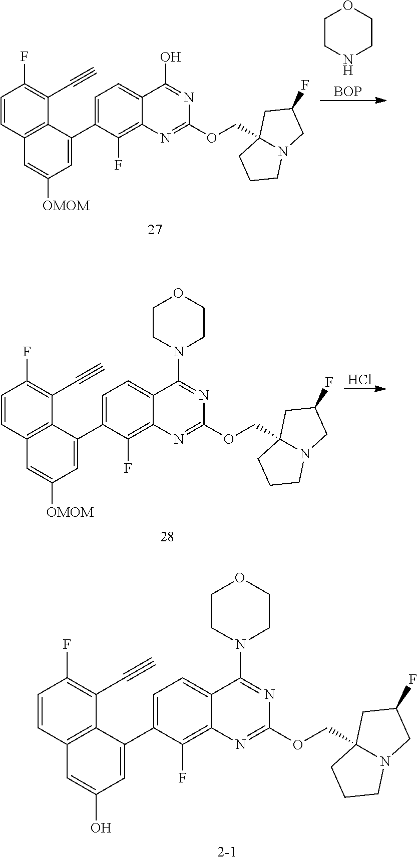

### Preparation of Intermediate 23: tert-butyl 3-(7-bromo-2-chloro-8-fluoroquinazolin-4-yl)-3,8-diazabicyclo[3.2.1]octane-8-carboxylate

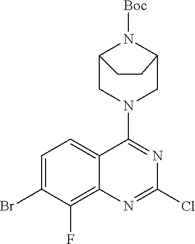 [SMILES: CC(C)(C)OC(=O)N1C2CCC1CN(c1nc(Cl)nc3c(F)c(Br)ccc13)C2]

To a stirred solution of commercially available 7-bromo-2,4-dichloro-8-fluoroquinazoline (15 g, 50.7 mmol) in 1,4-dioxane (150 mL) at 0° C. was added DIPEA (26.6 mL, 152 mmol) followed by tert-butyl 3,8-diazabicyclo[3.2.1]octane-8-carboxylate (12.9 g, 60.8 mmol). The reaction mixture was stirred at room temperature for 1 h. The reaction mixture was quenched with water and extracted with ethyl acetate. The combined organic layers were washed with brine, dried over anhydrous sodium sulfate, filtered, and evaporated under reduced pressure to provide the crude product. The crude compound was purified by COMBIFLASH™ chromatography (Teledyne ISO, Lincoln, NE) (120 g silica gel column, using 50 to 80% ethyl acetate/petroleum ether) to provide tert-butyl 3-(7-bromo-2-chloro-8-fluoroquinazolin-4-yl)-3,8-diazabicyclo[3.2.1]octane-8-carboxylate (17 g, 32.4 mmol, 64% yield) as a pale yellow solid. MS(ESI) m/z: 473.3 [M+2]⁺.

### Preparation of Intermediate 24: tert-butyl 3-(2-{[(2R,7aS)-2-fluoro-hexahydro-1H-pyrrolizin-7a-yl]methoxy}-7-bromo-8-fluoroquinazolin-4-yl)-3,8-diazabicyclo[3.2.1]octane-8-carboxylate

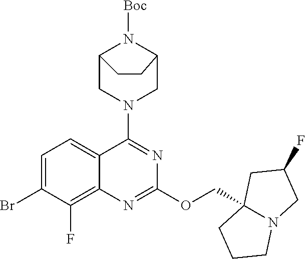 [SMILES: CC(C)(C)OC(=O)N1C2CCC1CN(c1nc(OC[C@@]34CCCN3C[C@H](F)C4)nc3c(F)c(Br)ccc13)C2]

To a solution of tert-butyl 3-(7-bromo-2-chloro-8-fluoroquinazolin-4-yl)-3,8-diazabicyclo[3.2.1]octane-8-carboxylate (3.95 g, 24.80 mmol) in THF (45 mL) at 0° C. was added NaH (0.194 g, 4.85 mmol, 60% w/w) and the reaction mixture was stirred at same temperature for 1 h. To this was added tert-butyl 3-(7-bromo-2-chloro-8-fluoroquinazolin-4-yl)-3,8-diazabicyclo[3.2.1]octane-8-carboxylate (9 g, 19.08 mmol) was added portion wise (4 Portions) at 0° C. Reaction mixture was allowed to warm to room temperature and stirred for 16 h. The reaction mixture was quenched with ice cold water and extracted with EtOAc (250 mL×2). The organic layer was washed with brine solution, dried over Na₂SO₄, filtered, and concentrated under reduced pressure to provide crude residue which was purified by COMBIFLASH™ chromatography (Teledyne ISO, Lincoln, NE) (240 g column, 80-100% EtOAc in petroluemether) to provide tert-butyl 3-(2-{[(2R,7aS)-2-fluoro-hexahydro-1H-pyrrolizin-7a-yl]methoxy}-7-bromo-8-fluoroquinazolin-4-yl)-3,8-diazabicyclo[3.2.1]octane-8-carboxylate (6.5 g, 10.93 mmol, 57% yield). MS(ESI) m/z: 594.5 (M+2)⁺.

### Preparation of Intermediate 25: tert-butyl 3-(2-{[(2R,7aS)-2-fluoro-hexahydro-1H-pyrrolizin-7a-yl]methoxy}-8-fluoro-7-[7-fluoro-3-(methoxymethoxy)-8-{2-[tris(propan-2-yl)silyl]ethynyl}naphthalen-1-yl]quinazolin-4-yl)-3,8-diazabicyclo[3.2.1]octane-8-carboxylate

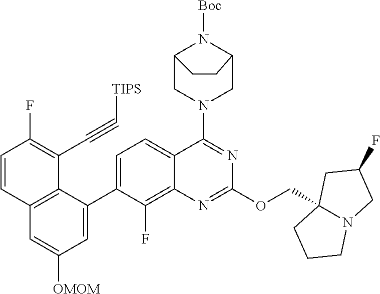

To a stirred solution of 3-(2-{[(2R,7aS)-2-fluoro-hexahydro-1H-pyrrolizin-7a-yl]methoxy}-7-bromo-8-fluoroquinazolin-4-yl)-3,8-diazabicyclo[3.2.1]octane-8-carboxylate (Intermediate 24, 8 g, 13.46 mmol) in 1,4-Dioxane (100 mL), were added ((2-fluoro-6-(methoxymethoxy)-8-(4,4,5,5-tetramethyl-1,3,2-dioxaborolan-2-yl)naphthalen-1-yl)ethynyl)triisopropylsilane (6.90 g, 13.46 mmol) and aqueous 2M potassium phosphate, tribasic (20.18 mL, 40.4 mmol). The reaction mixture was degassed under argon for 5 minutes, then [1,1′-bis(di-tertbutylphosphino)ferrocene]dichloropalladium(II) (0.877 g, 1.346 mmol) was added and again degassed under argon for 3 minutes and heated at 50° C. for 16 h. The reaction mixture was cooled to room temperature, diluted with water, and extracted with ethyl acetate. The combined organic layers were washed with brine, dried over anhydrous sodium sulfate, filtered, and concentrated under reduced pressure to provide a crude residue which was purified by COMBIFLASH™ chromatography (Teledyne ISO, Lincoln, NE) (80 g silica gel column, using 50 to 100% ethyl acetate/Petroleum ether) to provide tert-butyl 3-(2-{[(2R,7aS)-2-fluoro-hexahydro-1H-pyrrolizin-7a-yl]methoxy}-8-fluoro-7-[7-fluoro-3-(methoxymethoxy)-8-{2-[tris(propan-2-yl)silyl]ethynyl}naphthalen-1-yl]quinazolin-4-yl)-3,8-diazabicyclo[3.2.1]octane-8-carboxylate (7.5 g, 7.25 mmol, 54% yield) as a brown solid. MS(ESI) m/z: 900.4 [M+H]⁺.

### Preparation of Intermediate 26: tert-butyl 3-(2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoroquinazolin-4-yl)-3,8-diazabicyclo[3.2.1]octane-8-carboxylate

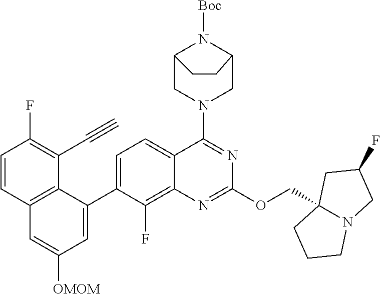

To a stirred solution of tert-butyl 3-(2-{[(2R,7aS)-2-fluoro-hexahydro-1H-pyrrolizin-7a-yl]methoxy}-8-fluoro-7-[7-fluoro-3-(methoxymethoxy)-8-{2-[tris(propan-2-yl)silyl]ethynyl}naphthalen-1-yl]quinazolin-4-yl)-3,8-diazabicyclo[3.2.1]octane-8-carboxylate (Intermediate 25, 3.7 g, 4.11 mmol) in DMF (50 mL) was added CsF (6.24 g, 41.1 mmol). The reaction mixture was heated at 50° C. for 1 h. The reaction mixture was then diluted with water and extracted with ethyl acetate. The combined organic layers were washed with brine, dried over anhydrous sodium sulfate, filtered, and concentrated under reduced pressure to provide the crude tert-butyl 3-(2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoroquinazolin-4-yl)-3,8-diazabicyclo[3.2.1]octane-8-carboxylate (2.9 g, 3.89 mmol, 95% yield) as a brown liquid, which was taken for next step without purification. MS(ESI) m/z: 744.3 [M+H]⁺.

### Preparation of Intermediate 27: 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoroquinazolin-4-ol

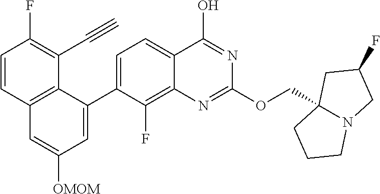

To a stirred solution of crude tert-butyl 3-(2-{[(2R,7aS)-2-fluoro-hexahydro-1H-pyrrolizin-7a-yl]methoxy}-7-[8-ethynyl-7-fluoro-3(methoxymethoxy)naphthalen-1-yl]-8-fluoroquinazolin-4-yl)-3,8-diazabicyclo[3.2.1]octane-8-carboxylate (Intermediate 26, 3 g, 4.03 mmol) in EtOH (15 mL) and THE (15 mL) was added NaOH (1M aq. soln.) (40.3 mL, 40.3 mmol). The reaction mixture was heated at 70° C. for 3h. The reaction mixture was evaporated, and the residue was diluted with ethyl acetate. The organic layer was washed with saturated NH₄Cl solution, water, brine, and then dried over anhydrous sodium sulfate, filtered, and concentrated under reduced pressure to provide the crude compound, which was purified by COMBIFLASH™ chromatography (Teledyne ISO, Lincoln, NE) (24 g silica gel column, using 10-20% MeOH/DCM) to provide 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoroquinazolin-4-ol (850 mg, 1.544 mmol, 38.3% yield) as a brown solid. MS(ESI) m/z: 550.2 [M+H]⁺.

### Preparation of Intermediate 28: 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoro-4-(morpholin-4-yl)quinazoline

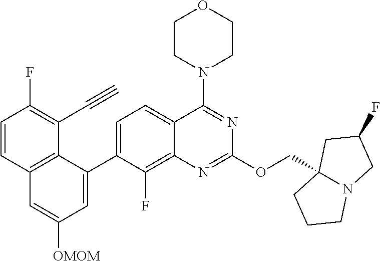

To a stirred solution of 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoroquinazolin-4-ol (Intermediate 27, 20 mg, 0.036 mmol) in ACN (1 mL) was added morpholine (3.8 mg, 0.044 mmol), triethylamine (0.020 mL, 0.146 mmol) and BOP (24.4 mg, 0.055 mmol). The reaction mixture was heated at 80° C. for 8h. The reaction mixture was evaporated, and the residue was diluted with ethyl acetate. The organic layer was washed with water and then brine and then dried over anhydrous sodium sulfate, filtered, and concentrated under reduced pressure to provide the crude 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoro-4-(morpholin-4-yl)quinazoline (25 mg, crude) as a brown liquid, which was taken for next step without purification. MS(ESI) m/z: 619.3 [M+H]⁺.

### Example 2-1: 4-(2-{[(2R,7aS)-2-fluoro-hexahydro-1H-pyrrolizin-7a-yl]methoxy}-8-fluoro-4-(morpholin-4-yl)quinazolin-7-yl)-5-ethynyl-6-fluoronaphthalen-2-ol

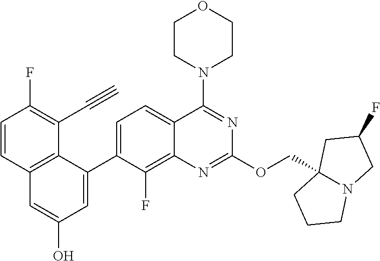 [SMILES: C#Cc1c(F)ccc2cc(O)cc(-c3ccc4c(N5CCOCC5)nc(OC[C@@]56CCCN5C[C@H](F)C6)nc4c3F)c12]

To a stirred solution of 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoro-4-(morpholin-4-yl)quinazoline (Intermediate 28, 200 mg, 0.323 mmol) in ACN (2 mL) at 0° C. was added 4N HCl (2 mL, 65.83 mmol in 1,4-dioxane) and the reaction mixture was stirred at room temperature for 1 h. The volatiles from the reaction mixture were removed under reduced pressure (at lower temperature, ˜30° C.) and the crude residue was co-evaporated with 1,4-dioxane. Then the crude residue was dissolved in 1,4-dioxane (5 mL) and TEA (1 mL) was added and then evaporated under reduced pressure to provide an off-white solid. The crude compound was purified by Prep-HPLC [HPLC Method: Preparative column: YMC EXRS (250×20×5 micron), Mobile phase A: 10 mM Ammonium bicarbonate in water-9.5; Mobile phase B: acetonitrile:methanol; Gradient=60-100% B over 20 minutes; Temperature: 27° C.; Flow rate: 19.0 mL/min; Detection: UV at 220 nm.] to provide 4-(2-{[(2R,7aS)-2-fluoro-hexahydro-1H-pyrrolizin-7a-yl]methoxy}-8-fluoro-4-(morpholin-4-yl)quinazolin-7-yl)-5-ethynyl-6-fluoronaphthalen-2-ol (11.9 mg, 0.020 mmol, 6.8% yield). MS(ESI) m/z: 575.1, [M+H]⁺. ¹H NMR (400 MHz, DMSO-d₆) δ ppm=7.96 (dd, J=9.1, 6.0 Hz, 1H), 7.76 (d, J=8.6 Hz, 1H), 7.46 (t, J=9.0 Hz, 1H), 7.37 (d, J=2.6 Hz, 1H), 7.24 (dd, J=8.4, 6.8 Hz, 1H), 7.09 (d, J=2.3 Hz, 1H), 5.38-5.19 (m, 1H), 4.13-4.08 (m, 1H), 4.01 (dd, J=10.4, 4.4 Hz, 1H), 3.88-3.72 (m, 9H), 3.16-3.00 (m, 3H), 2.88-2.78 (m, 1H), 2.19-1.97 (m, 3H), 1.88-1.73 (m, 3H).

### Preparation of Intermediate 29: 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoro-4-(1,4-oxazepan-4-yl)quinazoline

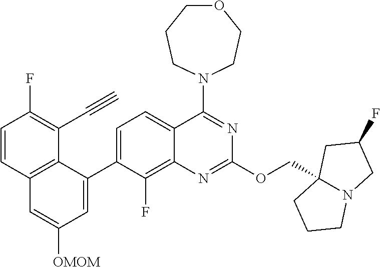

To a stirred solution of 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoroquinazolin-4-ol (Intermediate 27, 300 mg, 0.546 mmol) in ACN (10 mL) was added 1,4-oxazepane (60.7 mg, 0.600 mmol), DIPEA (0.286 mL, 1.635 mmol) and BOP (362 mg, 0.817 mmol). The reaction mixture was heated at 80° C. for 8 h. The reaction mixture was cooled to room temperature, the solvent was evaporated under reduced pressure and the residue was diluted with ethyl acetate. The organic layer was washed with water and brine and then dried over anhydrous sodium sulfate, filtered, and concentrated under reduced pressure to provide the crude residue. The crude material was purified by COMBIFLASH™ chromatography (Teledyne ISO, Lincoln, NE) (40 g REDISEP™ column (Teledyne ISCO, Lincoln, NE), eluting with a gradient from 60-100% EtOAc in petroleum ether) to provide 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoro-4-(1,4-oxazepan-4-yl)quinazoline (300 mg, 0.474 mmol, 87% yield) as a brown liquid. MS(ESI) m/z: 633.3 [M+H]⁺.

### Example 2-2: 4-(2-{[(2R,7aS)-2-fluoro-hexahydro-1H-pyrrolizin-7a-yl]methoxy}-8-fluoro-4-(1,4-oxazepan-4-yl)quinazolin-7-yl)-5-ethynyl-6-fluoronaphthalen-2-ol

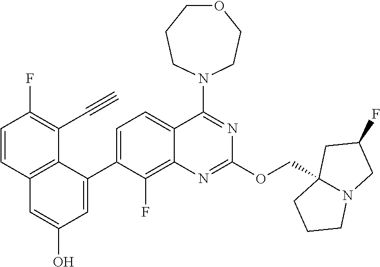 [SMILES: C#Cc1c(F)ccc2cc(O)cc(-c3ccc4c(N5CCCOCC5)nc(OC[C@@]56CCCN5C[C@H](F)C6)nc4c3F)c12]

To a stirred solution of 2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoro-4-(1,4-oxazepan-4-yl)quinazoline (Intermediate 29, 300 mg, 0.474 mmol) in acetonitrile (5 mL) at 0° C. was added HCl (4M in dioxane) (0.15 mL, 4.74 mmol) and the reaction mixture was stirred at room temperature for 1 h. The volatiles from the reaction mixture were removed under reduced pressure (at lower temperature, ˜30° C.) and the crude residue was co-evaporated with 1,4-dioxane. Then the crude residue was dissolved in 1,4-dioxane (2 mL) and excess TEA (1 mL) was added and evaporated under reduced pressure to provide a crude residue. The crude compound was purified by Prep-HPLC [HPLC Method: Column: YMC ExRS (20×250) 5 u; Mobile phase A: 10 mM ammonium bicarbonate in H₂O pH 9.5 Mobile phase B: ACN, Flow:19 mL/min Gradient T/% B: 0/50, 2/50; 15/70; 16/100 to provide 4-(2-{[(2R,7aS)-2-fluoro-hexahydro-1H-pyrrolizin-7a-yl]methoxy}-8-fluoro-4-(1,4-oxazepan-4-yl)quinazolin-7-yl)-5-ethynyl-6-fluoronaphthalen-2-ol (59.02 mg, 0.098 mmol, 21% yield). MS(ESI) m/z: 589.40 [M+H]⁺. ¹H NMR (400 MHz, DMSO-d₆) δ 10.23-10.08 (m, 1H), 7.97 (dd, J=9.3, 5.8 Hz, 1H), 7.83 (d, J=9.0 Hz, 1H), 7.46 (t, J=9.0 Hz, 1H), 7.36 (d, J=2.5 Hz, 1H), 7.18 (dd, J=8.5, 7.0 Hz, 1H), 7.07 (d, J=2.5 Hz, 1H), 5.35-5.22 (m, 1H), 4.12-3.90 (m, 8H), 3.77 (t, J=5.3 Hz, 2H), 3.09 (br d, J=13.5 Hz, 2H), 3.02 (s, 1H), 2.83 (br d, J=6.5 Hz, 1H), 2.58-2.53 (m, 1H), 2.16-2.08 (m, 3H), 2.08-1.98 (m, 2H), 1.89-1.73 (m, 3H).

### Preparation of Intermediate 30: tert-butyl 3-(2-{[(4aS,7aR)-1-methyl-octahydro-1H-cyclopenta[b]pyridin-4a-yl]methoxy}-7-bromo-8-fluoroquinazolin-4-yl)-3,8-diazabicyclo[3.2.1]octane-8-carboxylate

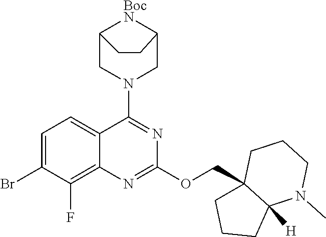 [SMILES: CN1CCC[C@@]2(COc3nc(N4CC5CCC(C4)N5C(=O)OC(C)(C)C)c4ccc(Br)c(F)c4n3)CCC[C@@H]12]

To a stirred solution of tert-butyl 3-(7-bromo-2-chloro-8-fluoroquinazolin-4-yl)-3,8-diazabicyclo[3.2.1]octane-8-carboxylate (Intermediate 23, 2.5 g, 5.49 mmol) and ((4aS,7aR)-1-methyloctahydro-4aH-cyclopenta[b]pyridin-4a-yl)methanol (Intermediate 12, 1.1 g, 6.59 mmol) in THE (25 mL) was added 1M LiHMDS (lithium hexamethyldisilazide) in hexanes (5.49 mL, 5.49 mmol) at 0° C. under nitrogen. The reaction mixture was allowed to warm to room temperature and was stirred for 15 h. The reaction mixture was quenched with saturated ammonium chloride solution (20 mL) and extracted with ethylacetate (3×). The combined organic extracts were washed with water, brine solution, dried over Na₂SO₄, filtered, and evaporated under reduced pressure to provide crude residue, which was purified by silica gel column chromatography using COMBIFLASH™ chromatography (Teledyne ISO, Lincoln, NE) (120 g REDISEP™ column (Teledyne ISCO, Lincoln, NE), 80 to 100% EtOAc-petroleum ether) to provide tert-butyl 3-(2-{[(4aS,7aR)-1-methyl-octahydro-1H-cyclopenta[b]pyridin-4a-yl]methoxy}-7-bromo-8-fluoroquinazolin-4-yl)-3,8-diazabicyclo[3.2.1]octane-8-carboxylate (2.5 g, 4.14 mmol, 75% yield), as a pale yellow solid. MS(ESI) m/z: 604.2 (M+H)*.

### Preparation of Intermediate 31: tert-butyl 3-(2-{[(4aS,7aR)-1-methyl-octahydro-1H-cyclopenta[b]pyridin-4a-yl]methoxy}-8-fluoro-7-[7-fluoro-3-(methoxymethoxy)-8-{2-[tris(propan-2-yl)silyl]ethynyl}naphthalen-1-yl]quinazolin-4-yl)-3,8-diazabicyclo[3.2.1]octane-8-carboxylate

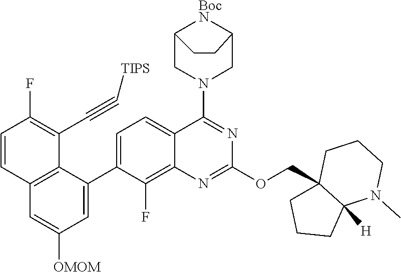

To a degassed stirred solution of tert-butyl 3-(2-{[(4aS,7aR)-1-methyl-octahydro-1H-cyclopenta[b]pyridin-4a-yl]methoxy}-7-bromo-8-fluoroquinazolin-4-yl)-3,8-diazabicyclo[3.2.1]octane-8-carboxylate (Intermediate 30, 2.5 g, 4.14 mmol) in 1,4-dioxane (50 mL), were added ((2-fluoro-6-(methoxymethoxy)-8-(4,4,5,5-tetramethyl-1,3,2-dioxaborolan-2-yl)naphthalen-1-yl)ethynyl)triisopropylsilane (2.119 g, 4.14 mmol) and aqueous 2M potassium phosphate tribasic (6.20 mL, 12.41 mmol) followed by [1,1′-bis(di-tertbutylphosphino)ferrocene]dichloropalladium(II) (0.135 g, 0.207 mmol). The reaction mixture was purged with N2 for 5 minutes and heated at 50° C. for 18h. The reaction mixture was cooled to room temperature, diluted with water, and extracted with ethyl acetate. The combined organic layers were washed with brine, dried over anhydrous sodium sulfate, filtered, and concentrated under reduced pressure to provide the crude product, which was purified by COMBIFLASH™ chromatography (Teledyne ISO, Lincoln, NE) (using 80 g silica gel column, using 50 to 100% ethyl acetate/Petroleum ether) to provide tert-butyl 3-(2-{[(4aS,7aR)-1-methyl-octahydro-1H-cyclopenta[b]pyridin-4a-yl]methoxy}-8-fluoro-7-[7-fluoro-3-(methoxymethoxy)-8-{2-[tris(propan-2-yl)silyl]ethynyl}naphthalen-1-yl]quinazolin-4-yl)-3,8-diazabicyclo[3.2.1]octane-8-carboxylate (3.0 g, 3.30 mmol, 80% yield) as a brown solid. MS(ESI) m/z: 911.2 [M+H]⁺.

### Preparation of Intermediate 32: tert-butyl 3-(2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoroquinazolin-4-yl)-3,8-diazabicyclo[3.2.1]octane-8-carboxylate

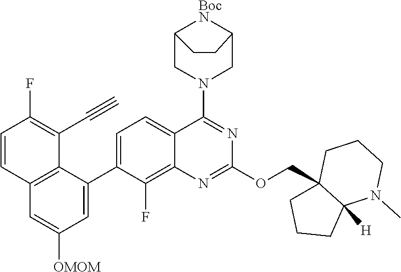

To a stirred solution of tert-butyl 3-(2-{[(4aS,7aR)-1-methyl-octahydro-1H-cyclopenta[b]pyridin-4a-yl]methoxy}-8-fluoro-7-[7-fluoro-3-(methoxymethoxy)-8-{2-[tris(propan-2-yl)silyl]ethynyl}naphthalen-1-yl]quinazolin-4-yl)-3,8-diazabicyclo[3.2.1]octane-8-carboxylate (Intermediate 31, 3.0 g, 3.30 mmol) in DMF (30 mL) was added cesium fluoride (5.01 g, 33.0 mmol) and the mixture was heated at 50° C. for 1 h. The reaction mixture was cooled to room temperature, diluted with water, and extracted with ethyl acetate. The combined organic layers were washed with brine, dried over anhydrous sodium sulfate, filtered, and concentrated under reduced pressure to provide tert-butyl 3-(2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoroquinazolin-4-yl)-3,8-diazabicyclo[3.2.1]octane-8-carboxylate. (2.2 g, 2.92 mmol, 89% yield) as a brown liquid, which was taken for next step without further purification. MS(ESI) m/z: 754.3 [M+H]⁺.

### Preparation of Intermediate 33: 2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoroquinazolin-4-ol

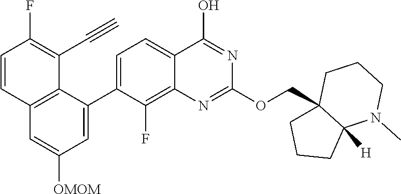

To a stirred solution of tert-butyl 3-(2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoroquinazolin-4-yl)-3,8 diazabicyclo[3.2.1]octane-8-carboxylate (Intermediate 32, 2.2 g, 2.92 mmol) in ethanol (20 mL) and THE (20 mL) was added NaOH (29.2 mL, 29.2 mmol) and the mixture was stirred at 80° C. for 36 h. The reaction mixture was then diluted with EtOAc (100 mL) and washed with aq. 0.5N HCl solution (150 mL×3). The aqueous layer was made basic (pH-8) with solid NaHCO₃and extracted with EtOAc (3×). The combined organic extracts were washed with water, brine solution, dried over anhydrous sodium sulfate, filtered, and evaporated under reduced pressure to provide the crude compound which was purified by COMBIFLASH™ chromatography (Teledyne ISO, Lincoln, NE) (40 g silicagel column, using 10-20% MeOH/DCM) to provide 2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoroquinazolin-4-ol. (1.5 g, 2.68 mmol, 92% yield) as a brown solid. MS(ESI) m/z: 560.2 [M+H]⁺.

### Preparation of Intermediate 34: 2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoro-4-(1,4-oxazepan-4-yl)quinazoline

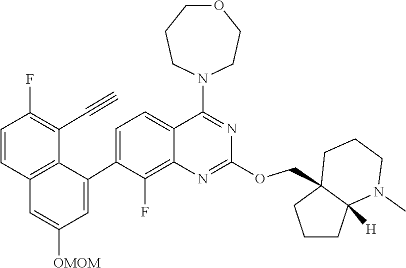

To a stirred solution of 2-{[(4aS)-1-methyl-octahydro-1H-cyclopenta[b]pyridin-4a-yl]methoxy}-7-[8-ethynyl-7-fluoro-3,3-bis(methoxymethoxy)naphthalen-1-yl]-8-fluoroquinazolin-4-ol (Intermediate 33, 255 mg, 0.447 mmol) in ACN (5 mL) was added 1,4-oxazepane (54.2 mg, 0.536 mmol), DIPEA (0.234 mL, 1.340 mmol) and BOP (296 mg, 0.670 mmol). The reaction mixture was heated at 80° C. for 8 h. The reaction mixture was cooled to room temperature, the solvent was evaporated under reduced pressure and the residue was diluted with ethyl acetate. The organic layer was washed with water, brine, dried over anhydrous sodium sulfate, filtered, and concentrated under reduced pressure to provide the crude residue and the crude material was purified by COMBIFLASH™ chromatography (Teledyne ISO, Lincoln, NE) (40 g REDISEP™ column (Teledyne ISCO, Lincoln, NE), eluting with a gradient from 60-100% EtOAc in petroleum ether) to provide (2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoro-4-(1,4-oxazepan-4-yl)quinazoline (211 mg, 73% yield) as a brown liquid. MS(ESI) m/z: 643.4 [M+H]⁺.

### Example 2-3: 4-(2-{[(4aS,7aR)-1-methyl-octahydro-1H-cyclopenta[b]pyridin-4a-yl]methoxy}-8-fluoro-4-(1,4-oxazepan-4-yl)quinazolin-7-yl)-5-ethynyl-6-fluoronaphthalen-2-ol

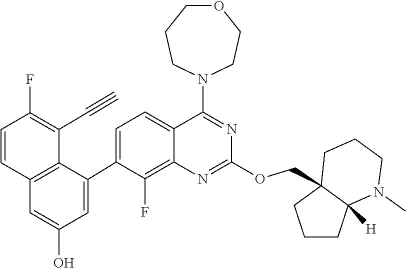 [SMILES: C#Cc1c(F)ccc2cc(O)cc(-c3ccc4c(N5CCCOCC5)nc(OC[C@]56CCC[C@H]5N(C)CCC6)nc4c3F)c12]

To a stirred solution of (2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoro-4-(1,4-oxazepan-4-yl)quinazoline (Intermediate 34, 330 mg, 0.521 mmol) in acetonitrile (5 mL) at 0° C. was added 4N HCl (1.3 mL, 5.21 mmol in 1,4-dioxane) and the reaction mixture was stirred at room temperature for 1 h. The volatiles from the reaction mixture were removed under reduced pressure (at lower temperature, ˜30° C.) and the crude residue was co-evaporated with 1,4-dioxane. Then the crude residue was dissolved in 1,4-dioxane (2 mL) and excess TEA (1 mL) was added and the mixture was evaporated under reduced pressure to provide an off-white solid. The crude compound was purified by Prep-HPLC [HPLC Method: Preparative Column: Waters XBridge BEH C18 XP (50×2.1 mm) 2.5 m; Mobile Phase A: 5:95 acetonitrile:water with 10 mM NH40Ac; Mobile Phase B: 95:5 acetonitrile:water with 10 mM NH40Ac; Temperature: 50° C.; Gradient: 0-100% B over 3 minutes; Flow: 1.1 mL/min. Injection 2 conditions: Column: Waters XBridge BEH C18 XP(50×2.1 mm) 2.5 m; Mobile Phase A: 5:95 acetonitrile:water with 0.1% TFA; Mobile Phase B: 95:5 acetonitrile:water with 0.1% TFA; Temperature:50° C.; Gradient:0-100% B over 3 minutes; Flow: 1.1 mL/min. to provide 4-(2-{[(4aS,7aR)-1-methyl-octahydro-1H-cyclopenta[b]pyridin-4a-yl]methoxy}-8-fluoro-4-(1,4-oxazepan-4-yl)quinazolin-7-yl)-5-ethynyl-6-fluoronaphthalen-2-ol (28 mg, 0.047 mmol, 9% yield) MS(ESI) m/z: 599.3 [M+H]⁺. ¹H NMR (400 MHz, DMSO-d₆) δ ppm=10.13 (s, 1H), 7.97 (dd, J=9.1, 6.0 Hz, 1H), 7.84 (br d, J=9.0 Hz, 1H), 7.47 (t, J=9.0 Hz, 1H), 7.37 (d, J=2.5 Hz, 1H), 7.21 (br s, 1H), 7.07 (d, J=2.1 Hz, 1H), 4.10-4.05 (m, 5H), 3.94-3.90 (m, 3H), 3.80-3.75 (m, 2H), 3.18-1.16 (m, 1H). 2.84-2.61 (m, 3H), 2.60-2.53 (m, 2H), 2.38-2.24 (m, 2H), 2.21-2.00 (m, 2H), 1.91 (s, 2H), 1.65-1.50 (m, 6H), 1.24 (s, 1H).

The examples in Table 2 were prepared according to procedures described for Example 2-1 from appropriate starting materials.

TABLE 2

| | | | LCMS | |
|---|---|---|---|---|
| # | Structure | IUPAC | (M + H)⁺ | ¹H NMR |
| 2-4 | 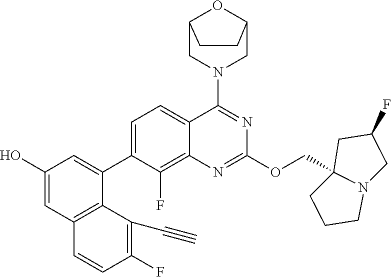 [SMILES: C#Cc1c(F)ccc2cc(O)cc(-c3ccc4c(N5CC6CCC(C5)O6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{8-oxa-3- azabicyclo[3.2.1]oc- tan-3-yl}quinazolin- 7-yl)-5-ethynyl-6- fluoronaphthalen-2- ol | 601.3 | ¹H NMR (400 MHz, DMSO-d₆) δ ppm = 10.18 (br s, 1H), 7.98 (dd, J = 9.2, 5.9 Hz, 1H), 7.83 (d, J = 8.8 Hz, 1H), 7.47 (t, J = 9.0 Hz, 1H), 7.38 (d, J = 2.5 Hz, 1H), 7.28 (dd, J = 8.5, 7.1 Hz, 1H), 7.07 (d, J = 2.5 Hz, 1H), 5.66 - 5.48 (m, 1H), 4.64 - 4.52 (m, 2H), 4.48 (br s, 2H), 4.36 - 4.25 (m, 2H), 3.88 - 3.75 (m, 4H), 3.66 - 3.59 (m, 2H), 3.34 - 3.26 (m, 1H), 3.15 - 3.07 (m, 2H), 2.57 (br d, J = 4.0 Hz, 1H), 2.24 - 2.12 (m, 2H), 2.10 - 1.98 (m, 1H), 1.92 - 1.80 (m, 2H), 1.18 (t, J = 7.3 |
| | | | | Hz, 2H). |
| 2-5 | 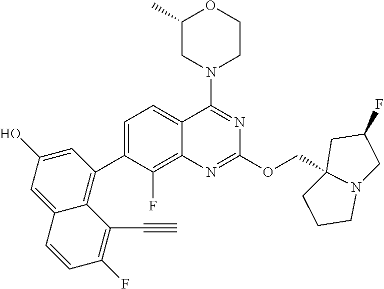 [SMILES: C#Cc1c(F)ccc2cc(O)cc(-c3ccc4c(N5CCO[C@@H](C)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(2S)-2- methylmorpholin-4- yl]quinazolin-7-yl)- 5-ethynyl-6- fluoronaphthalen-2- ol | 589.3 | ¹H NMR (400 MHz, DMSO-d₆) δ ppm = 10.13 (br s, 1H), 7.97 (dd, J = 5.9, 9.2 Hz, 1H), 7.76 (d, J = 8.4 Hz, 1H), 7.46 (t, J = 9.0 Hz, 1H), 7.37 (d, J = 2.4 Hz, 1H), 7.28 - 7.21 (m, 1H), 7.08 (d, J = 2.4 Hz, 1H), 5.38 - 5.19 (m, 1H), 4.25 - 4.09 (m, 3H), 4.05 - 3.93 (m, 2H), 3.87 (d, J = 2.3 Hz, 1H), 3.83 - 3.72 (m, 2H), 3.19 - 2.96 (m, 5H), 2.90 - 2.80 (m, 1H), 2.18 - 2.00 (m, 3H), 1.90 - 1.75 (m, 3H), 1.19 (d, J = 6.3 Hz, 3H). |
| 2-6 | 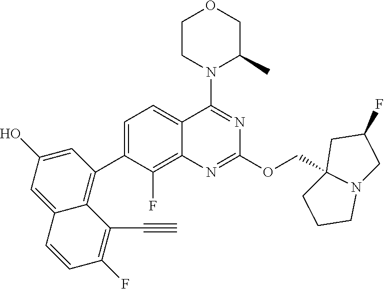 [SMILES: C#Cc1c(F)ccc2cc(O)cc(-c3ccc4c(N5CCOC[C@H]5C)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(3R)-3- methylmorpholin-4- yl]quinazolin-7-yl)- 5-ethynyl-6- fluoronaphthalen-2- ol | 589.2 | ¹H NMR (400 MHz, DMSO-d₆) δ ppm = 10.13 (br s, 1H), 7.96 (dd, J = 5.9, 9.2 Hz, 1H), 7.72 (dd, J = 5.8, 8.4 Hz, 1H), 7.49 - 7.43 (m, 1H), 7.37 - 7.35 (m, 1H), 7.25 - 7.19 (m, 1H), 7.07 (d, J = 2.5 Hz, 1H), 5.36 - 5.20 (m, 1H), 4.71 - 4.51 (m, 1H), 4.12 - 3.92 (m, 4H), 3.85 (d, J = 15.6 Hz, 1H), 3.79 - 3.64 (m, 4H), 3.14 - 3.01 (m, 3H), 2.89 - 2.78 (m, 1H), 2.19 - 2.00 (m, 3H), 1.86 - 1.76 (m, 3H), 1.42 (dd, J = 6.8, 12.2 Hz, 3H) |
| 2-7 | 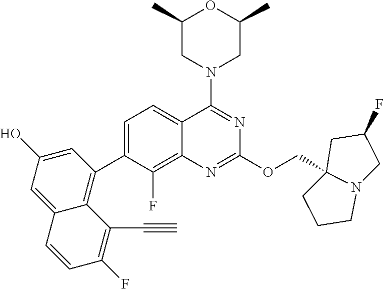 [SMILES: C#Cc1c(F)ccc2cc(O)cc(-c3ccc4c(N5C[C@@H](C)O[C@@H](C)C5)nc(OC[C@@]56CCCN5C[C@H](F)C6)nc4c3F)c12] | 4-(2-{[(2R,7aS)-2- fluoro-hexahydro- 1H-pyrrolizin- 7a-yl]methoxy}-4- [(2R,6S)-2,6- dimethylmorpholin- 4- yl]-8- fluoroquinazolin-7- yl)-5-ethynyl-6- fluoronaphthalen-2- ol | 603.3 | ¹H NMR (400 MHz, DMSO-d₆) δ ppm = 10.12 (s, 1H), 7.99 - 7.94 (m, 1H), 7.76 (d, J = 8.6 Hz, 1H), 7.49 - 7.43 (m, 1H), 7.36 (d, J = 2.5 Hz, 1H), 7.27 - 7.22 (m, 1H), 7.07 (d, J = 2.0 Hz, 1H), 5.38 - 5.20 (m, 1H), 4.21 (br t, J = 12.4 Hz, 2H), 4.16 - 4.09 (m, 1H), 4.06 - 3.99 (m, 1H), 3.91 - 3.78 (m, 4H), 3.19 - 3.03 (m, 3H), 2.96 - 2.82 (m, 3H), 2.17 - 2.01 (m, 3H), 1.89 - 1.75 (m, 3H), 1.19 (d, J = 6.1 Hz, 6H) |
| 2-8 | 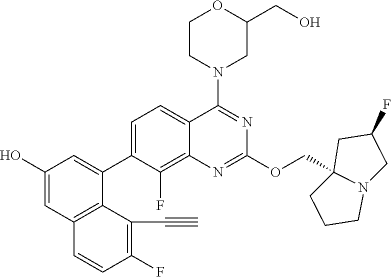 [SMILES: C#Cc1c(F)ccc2cc(O)cc(-c3ccc4c(N5CCOC(CO)C5)nc(OC[C@@]56CCCN5C[C@H](F)C6)nc4c3F)c12] | 4-(2-{[(2R,7aS)-2- fluoro-hexahydro- 1H-pyrrolizin- 7a-yl]methoxy}-8- fluoro-4-[2- (hydroxymethyl)mor- pholin-4- yl]quinazolin-7-yl)- 5- ethynyl-6- fluoronaphthalen-2- ol | 605.3 | ¹H NMR (400 MHz, DMSO-d₆) δ ppm = 10.14 (s, 1H), 7.97 (dd, J = 6.0, 9.0 Hz, 1H), 7.77 (d, J = 8.8 Hz, 1H), 7.46 (t, J = 9.0 Hz, 1H), 7.37 (d, J = 2.5 Hz, 1H), 7.26- 7.22 (m, 1H), 7.09 (t, J = 2.4 Hz, 1H), 5.47 - 5.19 (m, 1H), 4.89 (br s, 1H), 4.37 - 4.15 (m, 2H), 4.14 - 4.07 (m, 1H), 4.05 - 3.94 (m, 2H), 3.90(s, 1H), 3.88 - 3.74 (m, 3H), 3.60 - 3.50 (m, 1H), 3.49 - 3.43 (m, 1H), 3.23 - 3.02 (m, 4H), 2.89 (br s, 1H), 2.22 - 1.99 (m, 3H), 1.91 - 1.80 (m, 3H) |
| 2-9 | 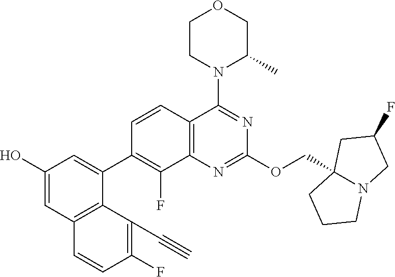 [SMILES: C#Cc1c(F)ccc2cc(O)cc(-c3ccc4c(N5CCOC[C@@H]5C)nc(OC[C@@]56CCCN5C[C@H](F)C6)nc4c3F)c12] | 4-(2-{[(2R,7aS)-2- fluoro-hexahydro- 1H-pyrrolizin- 7a-yl]methoxy}-8- fluoro-4-[(3S)-3- methylmorpholin-4- yl]quinazolin-7-yl)- 5-ethynyl-6- fluoronaphthalen-2- ol | 589.3 | ¹H NMR (400 MHz, DMSO-d₆) δ ppm = 10.14 (s, 1H), 7.96 (dd, J = 9.1, 6.1 Hz, 1H), 7.76 - 7.70 (m, 1H), 7.50 - 7.43 (m, 1H), 7.36 (s, 1H), 7.24 (br t, J = 7.6 Hz, 1H), 7.07 (s, 1H), 5.44 - 5.23 (m, 1H), 4.71 - 4.53 (m, 1H), 4.26 - 3.91 (m, 4H), 3.88 - 3.61 (m, 5H), 3.24 - 3.04 (m, 3H), 2.98 - 2.86 (m, 1H), 2.25 - 2.01 (m, 3H), 1.97 - 1.76 (m, 3H), 1.43 (dd, J = 10.8, 6.5 Hz, 3H). |
| 2-10 | 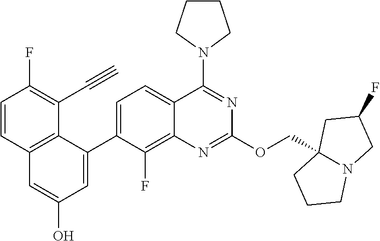 [SMILES: C#Cc1c(F)ccc2cc(O)cc(-c3ccc4c(N5CCCC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-(pyrrolidin- 1-yl)quinazolin-7- yl)-5-ethynyl-6- fluoronaphthalen-2- ol | 559 | |
| 2-11 | 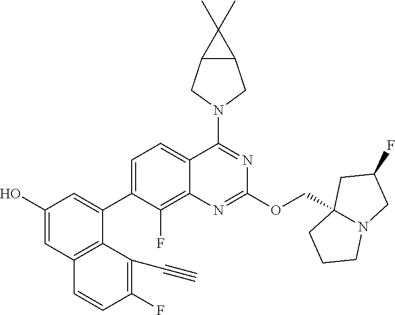 [SMILES: C#Cc1c(F)ccc2cc(O)cc(-c3ccc4c(N5CC6C(C5)C6(C)C)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-{6,6- dimethyl-3- azabicyclo[3.1.0]hex- an-3-yl}-8- fluoroquinazolin-7- yl)-5-ethynyl-6- fluoronaphthalen-2- ol | 599.3 | |
| 2-12 | 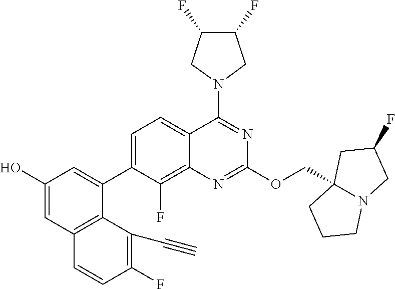 [SMILES: C#Cc1c(F)ccc2cc(O)cc(-c3ccc4c(N5C[C@@H](F)[C@@H](F)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4- [(3R,4S)-3,4- difluoropyrrolidin-1- yl]-8- fluoroquinazolin-7- yl)-5-ethynyl-6- fluoronaphthalen-2- ol | 595.2 | |
| 2-13 | 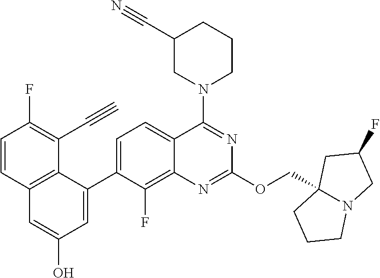 [SMILES: C#Cc1c(F)ccc2cc(O)cc(-c3ccc4c(N5CCCC(C#N)C5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)piperidine-3- carbonitrile | 598.3 | |
| 2-14 | 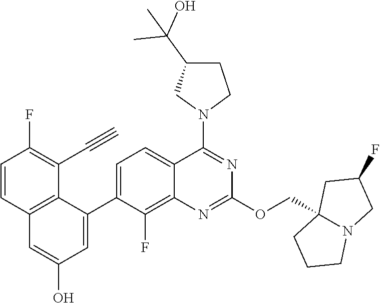 [SMILES: C#Cc1c(F)ccc2cc(O)cc(-c3ccc4c(N5CC[C@@H](C(C)(C)O)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(3R)-3-(2- hydroxypropan-2- yl)pyrrolidin-1- yl]quinazolin-7-yl)- 5-ethynyl-6- fluoronaphthalen-2- ol | 617.3 | |
| 2-15 | 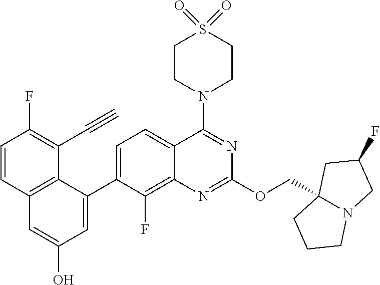 [SMILES: C#Cc1c(F)ccc2cc(O)cc(-c3ccc4c(N5CCS(=O)(=O)CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)-1¿6- thiomorpholine-1,1- dione | 623.2 | |
| 2-16 | 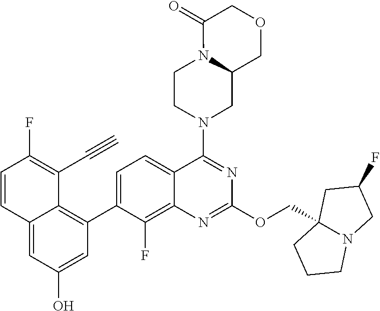 [SMILES: C#Cc1c(F)ccc2cc(O)cc(-c3ccc4c(N5CCN6C(=O)COC[C@H]6C5)nc(OC[C@@]56CCCN5C[C@H](F)C6)nc4c3F)c12] | (9aR)-8-(2- {[(2R,7aS)-2-fluoro- hexahydro-1H- pyrrolizin-7a- yl]methoxy}-7-(8- ethynyl-7-fluoro-3- hydroxynaphthalen- 1-yl)-8- fluoroquinazolin-4- yl)- octahydropiperazino [2,1-c]morpholin-4- one | 644.3 | |
| 2-17 | 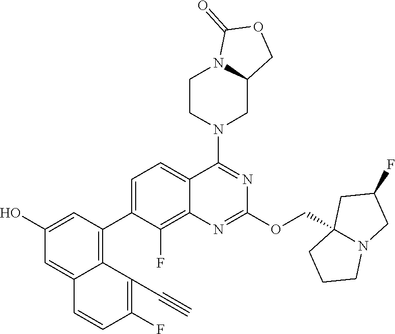 [SMILES: C#Cc1c(F)ccc2cc(O)cc(-c3ccc4c(N5CCN6C(=O)OC[C@H]6C5)nc(OC[C@@]56CCCN5C[C@H](F)C6)nc4c3F)c12] | (8aR)-7-(2- {[(2R,7aS)-2-fluoro- hexahydro-1H- pyrrolizin-7a- yl]methoxy}-7-(8- ethynyl-7-fluoro-3- hydroxynaphthalen- 1-yl)-8- fluoroquinazolin-4- yl)-hexahydro-1H- [1,3]oxazolo[3,4- a]piperazin-3-one | 630.3 | |
| 2-18 | 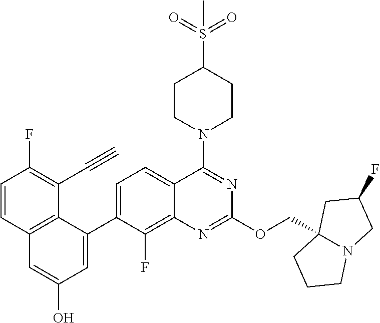 [SMILES: C#Cc1c(F)ccc2cc(O)cc(-c3ccc4c(N5CCC(S(C)(=O)=O)CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-(4- methanesulfonylpiper- idin-1- yl)quinazolin-7-yl)- 5-ethynyl-6- fluoronaphthalen-2- ol | 651.2 | |
| 2-19 | 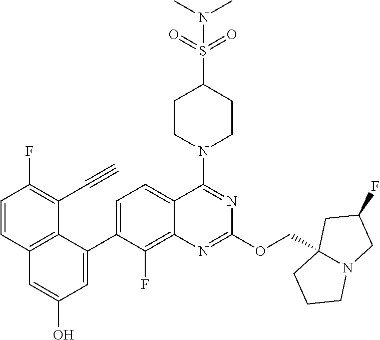 [SMILES: C#Cc1c(F)ccc2cc(O)cc(-c3ccc4c(N5CCC(S(=O)(=O)N(C)C)CC5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)-N,N- dimethylpiperidine- 4-sulfonamide | 680.2 | |
| 2-20 | 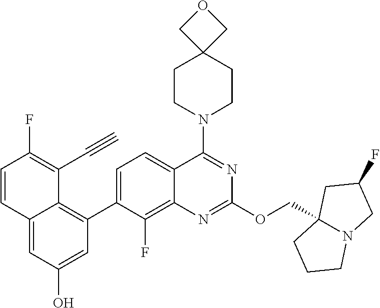 [SMILES: C#Cc1c(F)ccc2cc(O)cc(-c3ccc4c(N5CCC6(CC5)COC6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{2-oxa-7- azaspiro[3.5]nonan- 7-yl}quinazolin-7- yl)-5-ethynyl-6- fluoronaphthalen-2- ol | 615.2 | |
| 2-21 | 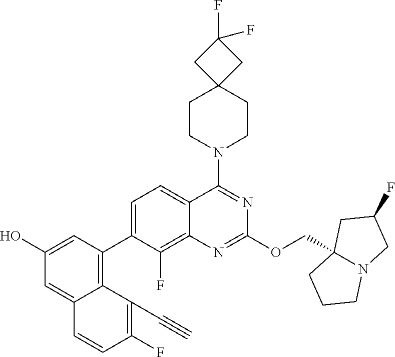 [SMILES: C#Cc1c(F)ccc2cc(O)cc(-c3ccc4c(N5CCC6(CC5)CC(F)(F)C6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-{2,2- difluoro-7- azaspiro[3.5]nonan- 7-yl}-8- fluoroquinazolin-7- yl)-5-ethynyl-6- fluoronaphthalen-2- ol | 649.3 | |
| 2-22 | 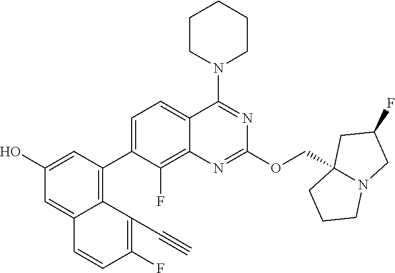 [SMILES: C#Cc1c(F)ccc2cc(O)cc(-c3ccc4c(N5CCCCC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-(piperidin- 1-yl)quinazolin-7- yl)-5-ethynyl-6- fluoronaphthalen-2- ol | 573.3 | |
| 2-23 | 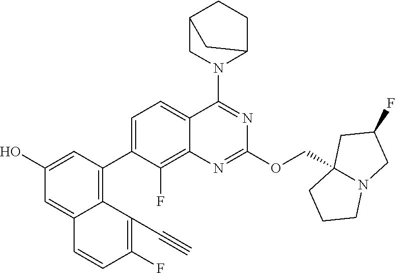 [SMILES: C#Cc1c(F)ccc2cc(O)cc(-c3ccc4c(N5CC6CCC5C6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-{2- azabicyclo[2.2.1]hep- tan-2-yl}-8- fluoroquinazolin-7- yl)-5-ethynyl-6- fluoronaphthalen-2- ol | 585.3 | |
| 2-24 | 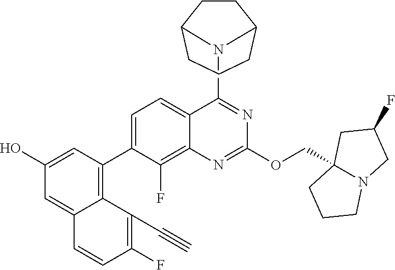 [SMILES: C#Cc1c(F)ccc2cc(O)cc(-c3ccc4c(N5C6CCCC5CC6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-{8- azabicyclo[3.2.1]oc- tan-8-yl}-8- fluoroquinazolin-7- yl)-5-ethynyl-6- fluoronaphthalen-2- ol | 599.3 | |
| 2-25 | 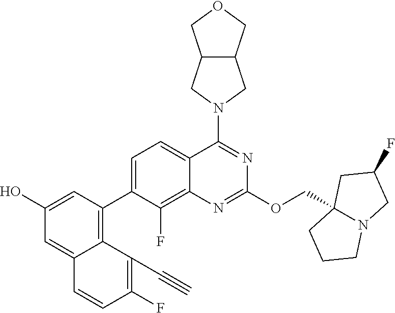 [SMILES: C#Cc1c(F)ccc2cc(O)cc(-c3ccc4c(N5CC6COCC6C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4- {hexahydro-1H- furo[3,4-c]pyrrol-5- yl}quinazolin-7-yl)- 5-ethynyl-6- fluoronaphthalen-2- ol | 601.3 | |
| 2-26 | 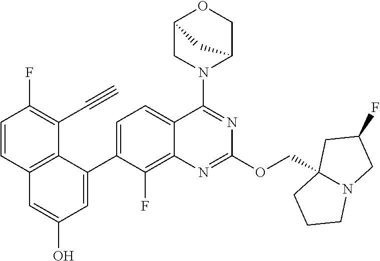 [SMILES: C#Cc1c(F)ccc2cc(O)cc(-c3ccc4c(N5C[C@@H]6C[C@H]5CO6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(1S,4S)-2- oxa-5- azabicyclo[2.2.1]hep- tan-5-yl]quinazolin- 7-yl)-5-ethynyl-6- fluoronaphthalen-2- ol | 587.3 | |
| 2-27 | 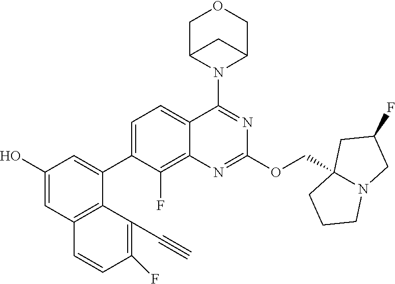 [SMILES: C#Cc1c(F)ccc2cc(O)cc(-c3ccc4c(N5C6COCC5C6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{3-oxa-6- azabicyclo[3.1.1]hep- tan-6-yl}quinazolin- 7-yl)-5-ethynyl-6- fluoronaphthalen-2- ol | 587.2 | ¹H NMR (400 MHz, DMSO-d₆) δ ppm = 10.12 (s, 1H), 7.97 (dd, J = 6.0, 9.1 Hz, 1H), 7.72 (d, J = 8.5 Hz, 1H), 7.49 - 7.44 (m, 1H), 7.36 (d, J = 2.5 Hz, 1H), 7.19 (dd, J = 6.9, 8.3 Hz, 1H), 7.07 (d, J = 2.3 Hz, 1H), 5.40 - 4.79 (m, 3H), 4.32 - 3.83 (m, 7H), 3.18 - 3.00 (m, 3H), 2.94 - 2.80 (m, 2H), 2.19 - 1.94 (m, 4H), 1.90 - 1.73 (m, 3H) |
| 2-28 | 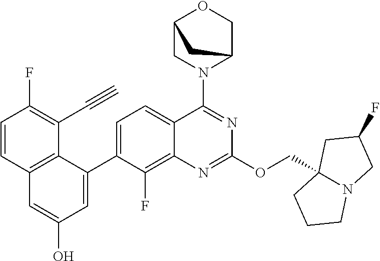 [SMILES: C#Cc1c(F)ccc2cc(O)cc(-c3ccc4c(N5C[C@H]6C[C@@H]5CO6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(1R,4R)-2- oxa-5- azabicyclo[2.2.1]hep- tan-5-yl]quinazolin- 7-yl)-5-ethynyl-6- fluoronaphthalen-2- ol | 587.2 | ¹H NMR (400 MHz, DMSO-d₆) δ ppm = 7.96 (dd, J = 6.0, 9.1 Hz, 1H), 7.91 - 7.84 (m, 1H), 7.49 - 7.42 (m, 1H), 7.37 - 7.34 (m, 1H), 7.21 - 7.14 (m, 1H), 7.06 (s, 1H), 5.35 (s, 1H), 5.32 - 5.19 (m, 1H), 4.78 - 4.74 (m, 1H), 4.23 - 4.15 (m, 1H), 4.13 - 4.08 (m, 1H), 4.01 - 3.79 (m, 6H), 3.11 - 3.00 (m, 3H), 2.87 - 2.79 (m, 1H), 2.16 - 2.00 (m, 4H), 1.96 - 1.91 (m, 1H), 1.86 - 1.81 (m, 1H), 1.80 - 1.76 (m, 1H) |
| 2-29 | 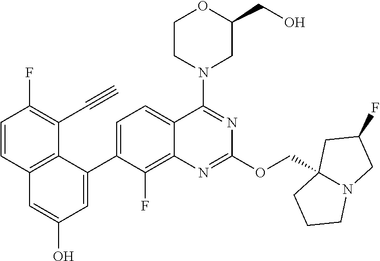 [SMILES: C#Cc1c(F)ccc2cc(O)cc(-c3ccc4c(N5CCO[C@@H](CO)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(2R)-2- (hydroxymethyl)mor- pholin-4- yl]quinazolin-7-yl)- 5-ethynyl-6- fluoronaphthalen-2- ol | 605.3 | |
| 2-30 | 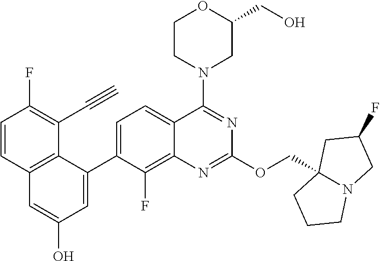 [SMILES: C#Cc1c(F)ccc2cc(O)cc(-c3ccc4c(N5CCO[C@H](CO)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(2S)-2- (hydroxymethyl)mor- pholin-4- yl]quinazolin-7-yl)- 5-ethynyl-6- fluoronaphthalen-2- ol | 605.3 | |
| 2-31 | 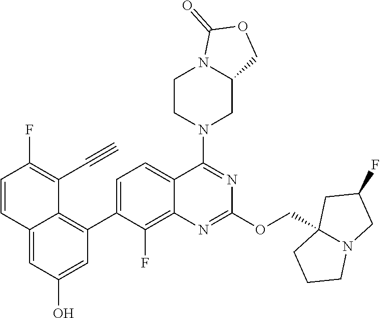 [SMILES: C#Cc1c(F)ccc2cc(O)cc(-c3ccc4c(N5CCN6C(=O)OC[C@@H]6C5)nc(OC[C@@]56CCCN5C[C@H](F)C6)nc4c3F)c12] | (8aS)-7-(2- {[(2R,7aS)-2-fluoro- hexahydro-1H- pyrrolizin-7a- yl]methoxy}-7-(8- ethynyl-7-fluoro-3- hydroxynaphthalen- 1-yl)-8- fluoroquinazolin-4- yl)-hexahydro-1H- [1,3]oxazolo[3,4- a]piperazin-3-one | 630.3 | |
| 2-32 | 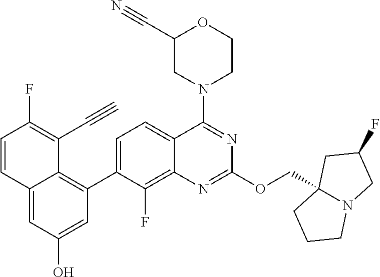 [SMILES: C#Cc1c(F)ccc2cc(O)cc(-c3ccc4c(N5CCOC(C#N)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)morpholine-2- carbonitrile | 600.2 | |
| 2-33 | 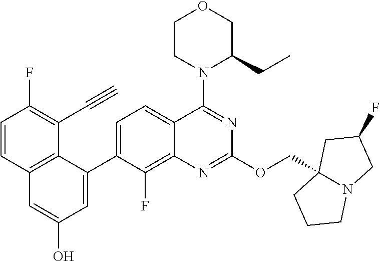 [SMILES: C#Cc1c(F)ccc2cc(O)cc(-c3ccc4c(N5CCOC[C@H]5CC)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4- [(3R)-3- ethylmorpholin-4- yl]-8- fluoroquinazolin-7- yl)-5-ethynyl-6- fluoronaphthalen-2- ol | 603.3 | |
| 2-34 | 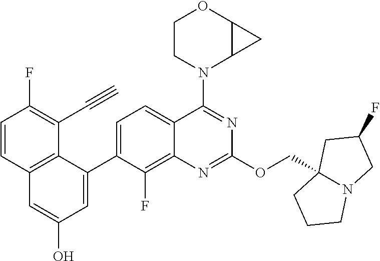 [SMILES: C#Cc1c(F)ccc2cc(O)cc(-c3ccc4c(N5CCOC6CC65)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{2-oxa-5- azabicyclo[4.1.0]hep- tan-5-yl}quinazolin- 7-yl)-5-ethynyl-6- fluoronaphthalen-2- ol | 587.2 | |
| 2-35 | 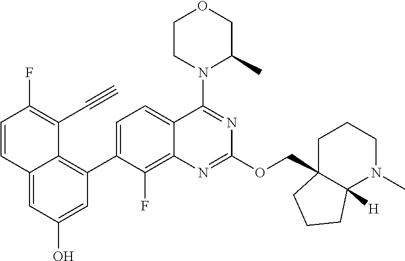 [SMILES: C#Cc1c(F)ccc2cc(O)cc(-c3ccc4c(N5CCOC[C@H]5C)nc(OC[C@]56CCC[C@H]5N(C)CCC6)nc4c3F)c12] | 4-(2-{[(4aS,7aR)-1- methyl-octahydro- 1H- cyclopenta[b]pyridin- 4a-yl]methoxy}-8- fluoro-4-[(3R)-3- methylmorpholin-4- yl]quinazolin-7-yl)- 5-ethynyl-6- fluoronaphthalen-2- ol | 599.3 | |
| 2-36 | 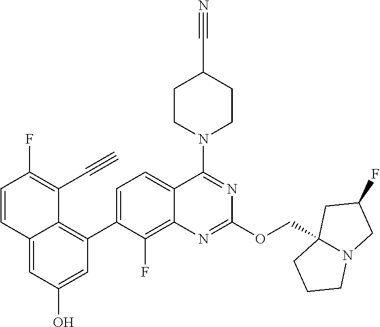 [SMILES: C#Cc1c(F)ccc2cc(O)cc(-c3ccc4c(N5CCC(C#N)CC5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)piperidine-4- carbonitrile | 598.2 | |
| 2-37 | 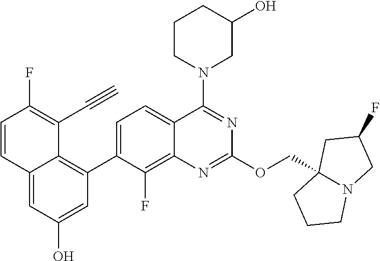 [SMILES: C#Cc1c(F)ccc2cc(O)cc(-c3ccc4c(N5CCCC(O)C5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)piperidin-3-ol | 589.3 | ¹H NMR (400 MHz, DMSO-d₆) δ ppm = 10.13 (s, 1H), 8.01 - 7.91 (m, 1H), 7.81 - 7.71 (m, 1H), 7.46 (s, 1H), 7.37 (d, J = 2.5 Hz, 1H), 7.28 - 7.18 (m, 1H), 7.12 -7.05 (m, 1H), 5.43 - 5.20 (m, 1H), 5.09 - 4.98 (m, 1H), 4.19 - 3.99 (m, 3H), 3.95 - 3.78 (m, 4H), 3.56 - 3.44 (m, 2H), 3.23 - 3.06 (m, 3H), 2.94 - 2.78 (m, 1H), 2.23 - 1.75 (m, 7H), 1.72 - 1.51 (m, 2H). |
| 2-38 | 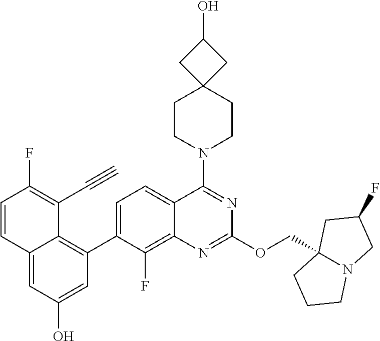 [SMILES: C#Cc1c(F)ccc2cc(O)cc(-c3ccc4c(N5CCC6(CC5)CC(O)C6)nc(OC[C@@]56CCCN5C[C@H](F)C6)nc4c3F)c12] | 7-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)-7- azaspiro[3.5]nonan- 2-ol | 629.3 | |
| 2-39 | 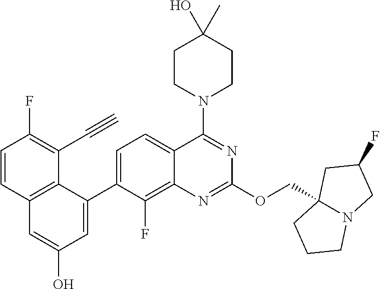 [SMILES: C#Cc1c(F)ccc2cc(O)cc(-c3ccc4c(N5CCC(C)(O)CC5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)-4- methylpiperidin-4-ol | 603.3 | |
| 2-40 | 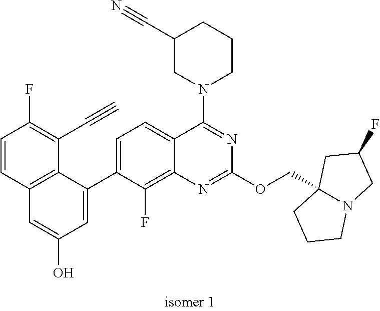 [SMILES: C#Cc1c(F)ccc2cc(O)cc(-c3ccc4c(N5CCCC(C#N)C5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)piperidine-3- carbonitrile | 598.35 | ¹H NMR (400 MHz, DMSO-d₆) δ ppm = 10.13 (s, 1H), 8.01 - 7.91 (m, 1H), 7.81 - 7.71 (m, 1H), 7.46 (s, 1H), 7.37 (d, J = 2.5 Hz, 1H), 7.28 - 7.18 (m, 1H), 7.12 -7.05 (m, 1H), 5.43 - 5.20 (m, 1H), 5.09 - 4.98 (m, 1H), 4.19 - 3.99 (m, 3H), 3.95 - 3.78 (m, 4H), 3.56 - 3.44 (m, 2H), 3.23 - 3.06 (m, 3H), 2.94 - 2.78 (m, 1H), 2.23 - 1.75 (m, 7H), 1.72 - 1.51 (m, 2H). |
| 2-41 | 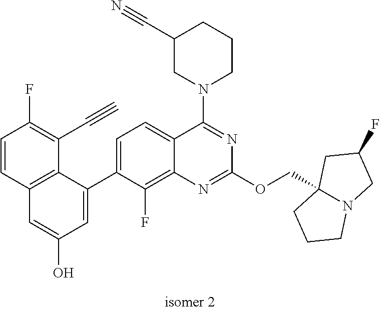 [SMILES: C#Cc1c(F)ccc2cc(O)cc(-c3ccc4c(N5CCCC(C#N)C5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)piperidine-3- carbonitrile | 598.3 | |
| 2-42 | 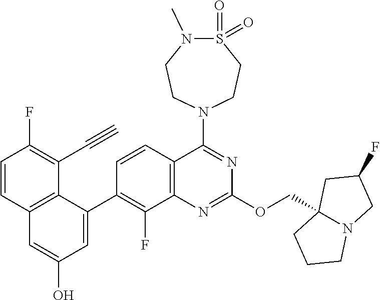 [SMILES: C#Cc1c(F)ccc2cc(O)cc(-c3ccc4c(N5CCN(C)S(=O)(=O)CC5)nc(OC[C@@]56CCCN5C[C@H](F)C6)nc4c3F)c12] | 5-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)-2-methyl- 1¿6,2,5- thiadiazepane-1,1- dione | 652.2 | |
| 2-43 | 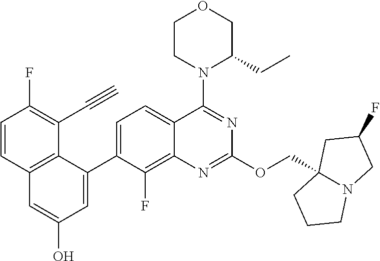 [SMILES: C#Cc1c(F)ccc2cc(O)cc(-c3ccc4c(N5CCOC[C@@H]5CC)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4- [(3S)-3- ethylmorpholin-4- yl]-8- fluoroquinazolin-7- yl)-5-ethynyl-6- fluoronaphthalen-2- ol | 603.1 | |
| 2-44 | 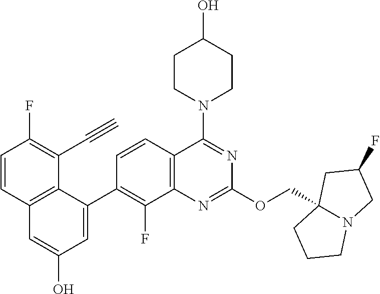 [SMILES: C#Cc1c(F)ccc2cc(O)cc(-c3ccc4c(N5CCC(O)CC5)nc(OC[C@@]56CCCN5C[C@H](F)C6)nc4c3F)c12] | 1-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)piperidin-4-ol | 589.3 | |
| 2-45 | 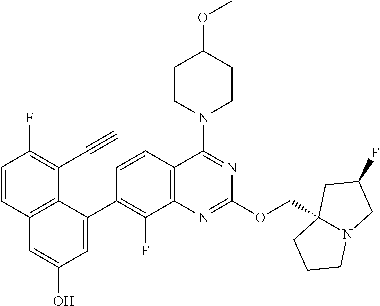 [SMILES: C#Cc1c(F)ccc2cc(O)cc(-c3ccc4c(N5CCC(OC)CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-(4- methoxypiperidin-1- yl)quinazolin-7-yl)- 5-ethynyl-6- fluoronaphthalen-2- ol | 603.3 | |
| 2-46 | 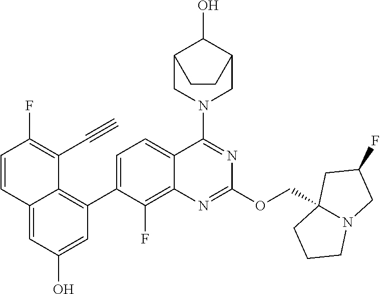 [SMILES: C#Cc1c(F)ccc2cc(O)cc(-c3ccc4c(N5CC6CCC(C5)C6O)nc(OC[C@@]56CCCN5C[C@H](F)C6)nc4c3F)c12] | 3-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)-3- azabicyclo[3.2.1]oc- tan-8-ol | 615.3 | |
| 2-47 | 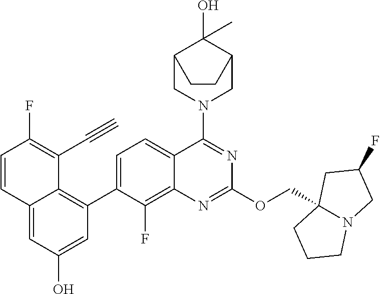 [SMILES: C#Cc1c(F)ccc2cc(O)cc(-c3ccc4c(N5CC6CCC(C5)C6(C)O)nc(OC[C@@]56CCCN5C[C@H](F)C6)nc4c3F)c12] | 3-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)-8-methyl-3- azabicyclo[3.2.1]oc- tan-8-ol | 629.3 | |
| 2-48 | 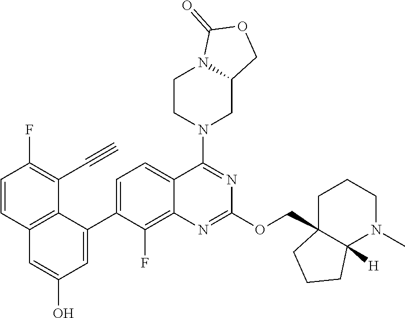 [SMILES: C#Cc1c(F)ccc2cc(O)cc(-c3ccc4c(N5CCN6C(=O)OC[C@@H]6C5)nc(OC[C@]56CCC[C@H]5N(C)CCC6)nc4c3F)c12] | (8aS)-7-(2- {[(4aS, 7aR)-1- methyl-octahydro- 1H- cyclopenta[b]pyridin- 4a-yl]methoxy}-7- (8-ethynyl-7-fluoro- 3- hydroxynaphthalen- 1-yl)-8- fluoroquinazolin-4- yl)-hexahydro-1H- [1,3]oxazolo[3,4- a]piperazin-3-one | 640.3 | |
| 2-49 | 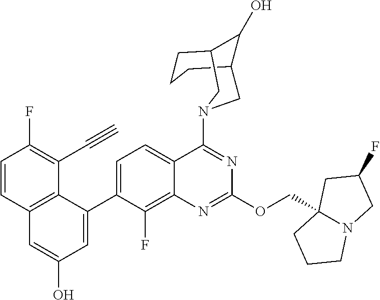 [SMILES: C#Cc1c(F)ccc2cc(O)cc(-c3ccc4c(N5CC6CCCC(C5)C6O)nc(OC[C@@]56CCCN5C[C@H](F)C6)nc4c3F)c12] | 3-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)-3- azabicyclo[3.3.1]no- nan-9-ol | 629.3 | |
| 2-50 | 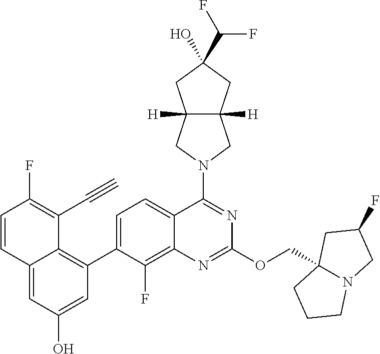 [SMILES: C#Cc1c(F)ccc2cc(O)cc(-c3ccc4c(N5C[C@@H]6C[C@@](O)(C(F)F)C[C@@H]6C5)nc(OC[C@@]56CCCN5C[C@H](F)C6)nc4c3F)c12] | (3aR,5R,6aS)-2-(2- {[(2R,7aS)-2-fluoro- hexahydro-1H- pyrrolizin-7a- yl]methoxy}-7-(8- ethynyl-7-fluoro-3- hydroxynaphthalen- 1-yl)-8- fluoroquinazolin-4- yl)-5- (difluoromethyl)- octahydrocyclopenta [c]pyrrol-5-ol | 665.3 | |
| 2-51 | 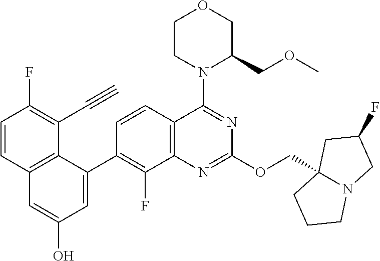 [SMILES: C#Cc1c(F)ccc2cc(O)cc(-c3ccc4c(N5CCOC[C@H]5COC)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(3R)-3- (methoxymethyl)mor- pholin-4- yl]quinazolin-7-yl)- 5-ethynyl-6- fluoronaphthalen-2- ol | 619.2 | |
| 2-52 | 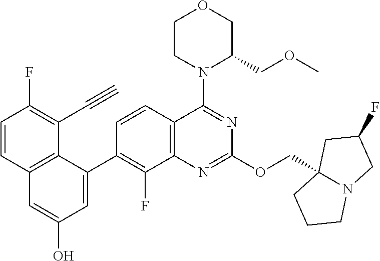 [SMILES: C#Cc1c(F)ccc2cc(O)cc(-c3ccc4c(N5CCOC[C@@H]5COC)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[(3S)-3- (methoxymethyl)mor- pholin-4- yl]quinazolin-7-yl)- 5-ethynyl-6- fluoronaphthalen-2- ol | 619.2 | |
| 2-53 | 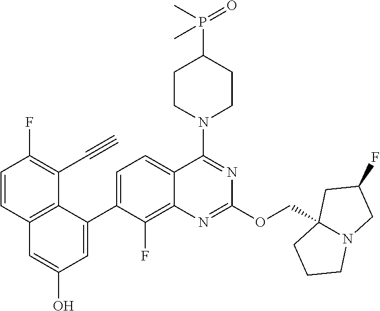 [SMILES: C#Cc1c(F)ccc2cc(O)cc(-c3ccc4c(N5CCC(P(C)(C)=O)CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-[4- (dimethylphosphoryl) piperidin-1-yl]-8- fluoroquinazolin-7- yl)-5-ethynyl-6- fluoronaphthalen-2- ol | 649.3 | |
| 2-54 | 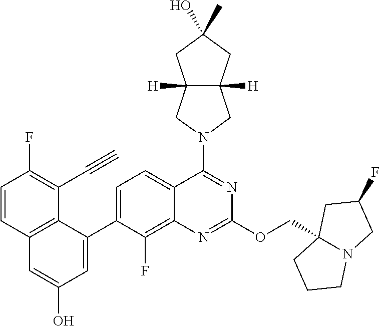 [SMILES: C#Cc1c(F)ccc2cc(O)cc(-c3ccc4c(N5C[C@@H]6C[C@](C)(O)C[C@@H]6C5)nc(OC[C@@]56CCCN5C[C@H](F)C6)nc4c3F)c12] | (3aR,5S,6aS)-2-(2- {[(2R, 7aS)-2-fluoro- hexahydro-1H- pyrrolizin-7a- yl]methoxy}-7-(8- ethynyl-7-fluoro-3- hydroxynaphthalen- 1-yl)-8- fluoroquinazolin-4- yl)-5-methyl- octahydrocyclopenta [c]pyrrol-5-ol | 629.3 | |
| 2-55 | 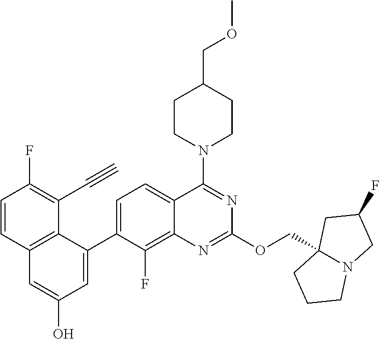 [SMILES: C#Cc1c(F)ccc2cc(O)cc(-c3ccc4c(N5CCC(COC)CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[4- (methoxymethyl)pi- peridin-1- yl]quinazolin-7-yl)- 5-ethynyl-6- fluoronaphthalen-2- ol | 617.3 | |
| 2-56 | 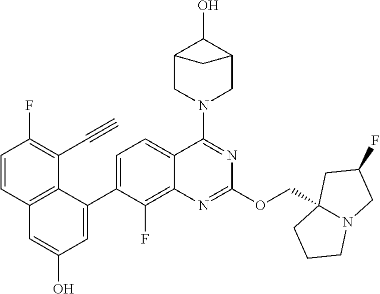 [SMILES: C#Cc1c(F)ccc2cc(O)cc(-c3ccc4c(N5CC6CC(C5)C6O)nc(OC[C@@]56CCCN5C[C@H](F)C6)nc4c3F)c12] | 3-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)-3- azabicyclo[3.1.1]hep- tan-6-ol | 601.3 | |
| 2-57 | 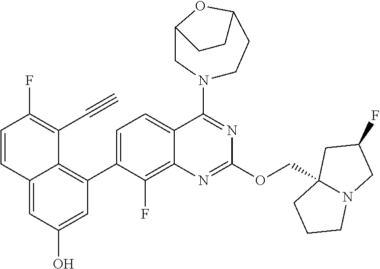 [SMILES: C#Cc1c(F)ccc2cc(O)cc(-c3ccc4c(N5CCC6CCC(C5)O6)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{9-oxa-3- azabicyclo[4.2.1 ]no- nan-3-yl}quinazolin- 7-yl)-5-ethynyl-6- fluoronaphthalen-2- ol | 615 | |
| 2-58 | 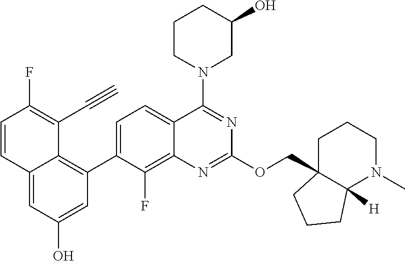 [SMILES: C#Cc1c(F)ccc2cc(O)cc(-c3ccc4c(N5CCC[C@@H](O)C5)nc(OC[C@]56CCC[C@H]5N(C)CCC6)nc4c3F)c12] | (3R)-1-(2- {[(4aS,7aR)-1- methyl-octahydro- 1H- cyclopenta[b]pyridin- 4a-yl]methoxy}-7- (8-ethynyl-7-fluoro- 3- hydroxynaphthalen- 1-yl)-8- fluoroquinazolin-4- yl)piperidin-3-ol | 599.3 | |
| 2-59 | 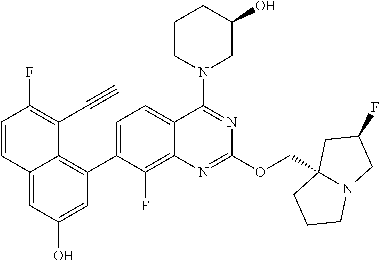 [SMILES: C#Cc1c(F)ccc2cc(O)cc(-c3ccc4c(N5CCC[C@@H](O)C5)nc(OC[C@@]56CCCN5C[C@H](F)C6)nc4c3F)c12] | (3R)-1-(2- {[(2R,7aS)-2-fluoro- hexahydro-1H- pyrrolizin-7a- yl]methoxy}-7-(8- ethynyl-7-fluoro-3- hydroxynaphthalen- 1-yl)-8- fluoroquinazolin-4- yl)piperidin-3-ol | 589.2 | |
| 2-60 | 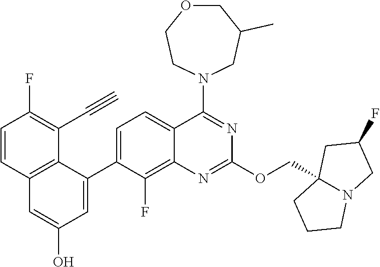 [SMILES: C#Cc1c(F)ccc2cc(O)cc(-c3ccc4c(N5CCOCC(C)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-(6-methyl- 1,4-oxazepan-4- yl)quinazolin-7-yl)- 5-ethynyl-6- fluoronaphthalen-2- ol | 603.2 | |
| 2-61 | 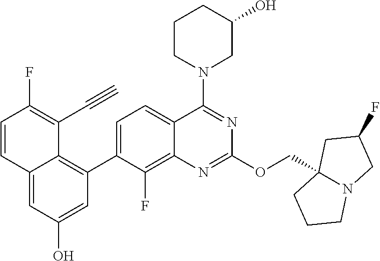 [SMILES: C#Cc1c(F)ccc2cc(O)cc(-c3ccc4c(N5CCC[C@H](O)C5)nc(OC[C@@]56CCCN5C[C@H](F)C6)nc4c3F)c12] | (3S)-1-(2- {[(2R,7aS)-2-fluoro- hexahydro-1H- pyrrolizin-7a- yl]methoxy}-7-(8- ethynyl-7-fluoro-3- hydroxynaphthalen- 1-yl)-8- fluoroquinazolin-4- yl)piperidin-3-ol | 589.2 | |
| 2-62 | 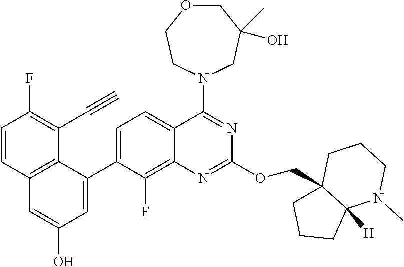 [SMILES: C#Cc1c(F)ccc2cc(O)cc(-c3ccc4c(N5CCOCC(C)(O)C5)nc(OC[C@]56CCC[C@H]5N(C)CCC6)nc4c3F)c12] | 4-(2-{[(4aS,7aR)-1- methyl-octahydro- 1H- cyclopenta[b]pyridin- 4a-yl]methoxy}-7- (8-ethynyl-7-fluoro- 3- hydroxynaphthalen- 1-yl)-8- fluoroquinazolin-4- yl)-6-methyl-1,4- oxazepan-6-ol | 629.3 | |
| 2-63 | 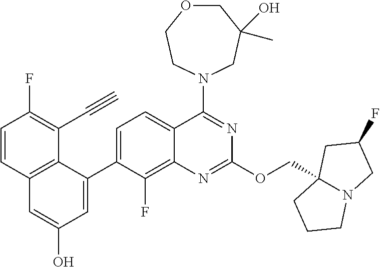 [SMILES: C#Cc1c(F)ccc2cc(O)cc(-c3ccc4c(N5CCOCC(C)(O)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)-6-methyl-1,4- oxazepan-6-ol | 619.1 | |
| 2-64 | 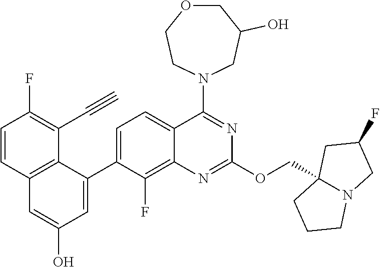 [SMILES: C#Cc1c(F)ccc2cc(O)cc(-c3ccc4c(N5CCOCC(O)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)-1,4-oxazepan-6- ol | 605.3 | |
| 2-65 | 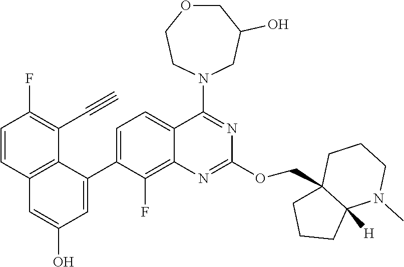 [SMILES: C#Cc1c(F)ccc2cc(O)cc(-c3ccc4c(N5CCOCC(O)C5)nc(OC[C@]56CCC[C@H]5N(C)CCC6)nc4c3F)c12] | 4-(2-{[(4aS,7aR)-1- methyl-octahydro- 1H- cyclopenta[b]pyridin- 4a-yl]methoxy}-7- (8-ethynyl-7-fluoro- 3- hydroxynaphthalen- 1-yl)-8- fluoroquinazolin-4- yl)-1,4-oxazepan-6- ol | 615.3 | |
| 2-66 | 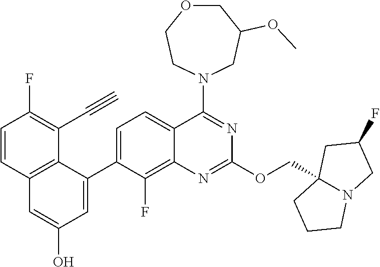 [SMILES: C#Cc1c(F)ccc2cc(O)cc(-c3ccc4c(N5CCOCC(OC)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-(6- methoxy-1,4- oxazepan-4- yl)quinazolin-7-yl)- 5-ethynyl-6- fluoronaphthalen-2- ol | 619.3 | |
| 2-67 | 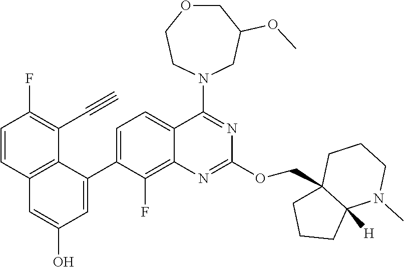 [SMILES: C#Cc1c(F)ccc2cc(O)cc(-c3ccc4c(N5CCOCC(OC)C5)nc(OC[C@]56CCC[C@H]5N(C)CCC6)nc4c3F)c12] | 4-(2-{[(4aS,7aR)-1- methyl-octahydro- 1H- cyclopenta[b]pyridin- 4a-yl]methoxy}-8- fluoro-4-(6- methoxy-1,4- oxazepan-4- yl)quinazolin-7-yl)- 5-ethynyl-6- fluoronaphthalen-2- ol | 629.3 | |
| 2-68 | 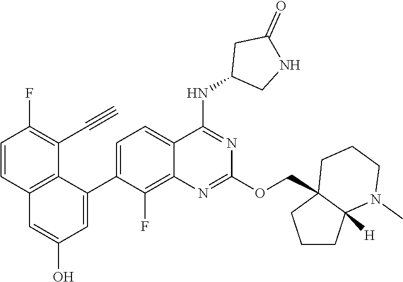 [SMILES: C#Cc1c(F)ccc2cc(O)cc(-c3ccc4c(N[C@H]5CNC(=O)C5)nc(OC[C@]56CCC[C@H]5N(C)CCC6)nc4c3F)c12] | (4R)-4-[(2- {[(4aS,7aR)-1- methyl-octahydro- 1H- cyclopenta[b]pyridin- 4a-yl]methoxy}-7- (8-ethynyl-7-fluoro- 3- hydroxynaphthalen- 1-yl)-8- fluoroquinazolin-4- yl)amino]pyrrolidin- 2-one | 598.2 | |
| 2-69 | 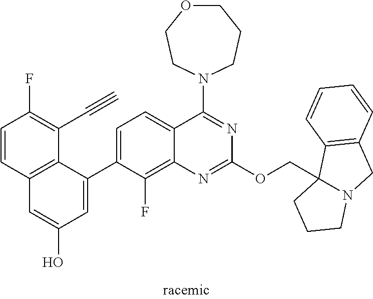 [SMILES: C#Cc1c(F)ccc2cc(O)cc(-c3ccc4c(N5CCCOCC5)nc(OCC56CCCN5Cc5ccccc56)nc4c3F)c12] | 4-[2- ({1H,2H,3H,5H,9bH- benzo[a]pyrrolizin- 9b-yl}methoxy)-8- fluoro-4-(1,4- oxazepan-4- yl)quinazolin-7-yl]- 5-ethynyl-6- fluoronaphthalen-2- ol | 619.2 | |
| 2-70 | 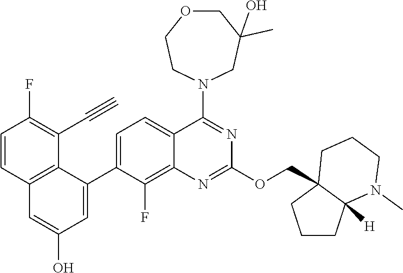 [SMILES: C#Cc1c(F)ccc2cc(O)cc(-c3ccc4c(N5CCOCC(C)(O)C5)nc(OC[C@]56CCC[C@H]5N(C)CCC6)nc4c3F)c12] | 4-(2-{[(4aS,7aR)-1- methyl-octahydro- 1H- cyclopenta[b]pyridin- 4a-yl]methoxy}-7- (8-ethynyl-7-fluoro- 3- hydroxynaphthalen- 1-yl)-8- fluoroquinazolin-4- yl)-6-methyl-1,4- oxazepan-6-ol | 629.2 | ¹H NMR (400 MHz, DMSO-d₆) δ ppm = 10.14 (s, 1H), 8.04 (d, J = 8.5 Hz, 1H), 7.97 (dd, J = 9.0, 6.3 Hz, 1H), 7.46 (t, J = 9.0 Hz, 1H), 7.36 (d, J = 2.5 Hz, 1H), 7.22 - 7.16 (m, 1H), 7.08 (t, J = 1.9 Hz, 1H), 5.31 - 5.14 (m, 1H), 4.46 - 4.06 (m, 3H), 4.06 - 3.74 (m, 5H), 3.56 - 3.53 (m, 4H), 2.68-2.67 (m, 1H), 2.22 - 2.18 (m, 4H), 1.89 - 1.70 (m, 2H), 1.60 - 1.52 (m, 7H), 1.45 - 1.33 (m, 1H), 1.17 - 1.51 (s, 3H). |
| 2-71 | 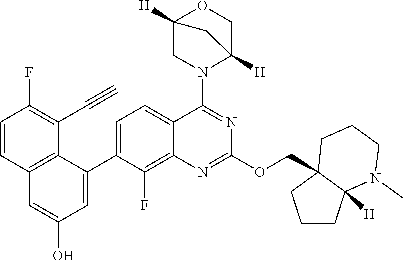 [SMILES: C#Cc1c(F)ccc2cc(O)cc(-c3ccc4c(N5C[C@H]6C[C@@H]5CO6)nc(OC[C@]56CCC[C@H]5N(C)CCC6)nc4c3F)c12] | 4-(2-{[(4aS,7aR)-1- methyl-octahydro- 1H- cyclopenta[b]pyridin- 4a-yl]methoxy}-8- fluoro-4-[(1R,4R)-2- oxa-5- azabicyclo[2.2.1]hep- tan-5-yl]quinazolin- 7-yl)-5-ethynyl-6- fluoronaphthalen-2- ol | 597.1 | |
| 2-72 | 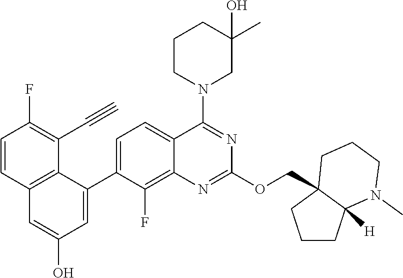 [SMILES: C#Cc1c(F)ccc2cc(O)cc(-c3ccc4c(N5CCCC(C)(O)C5)nc(OC[C@]56CCC[C@H]5N(C)CCC6)nc4c3F)c12] | 1-(2-{[(4aS,7aR)-1- methyl-octahydro- 1H- cyclopenta[b]pyridin- 4a-yl]methoxy}-7- (8-ethynyl-7-fluoro- 3- hydroxynaphthalen- 1-yl)-8- fluoroquinazolin-4- yl)-3- methylpiperidin-3-ol | 613.2 | ¹H NMR (400 MHz, DMSO-d₆) δ ppm = 10.26 - 10.01 (m, 1H), 7.96 (dd, J = 9.2, 5.9 Hz, 1H), 7.90 - 7.75 (m, 1H), 7.51 - 7.42 (m, 1H), 7.39 - 7.32 (m, 1H), 7.24 - 7.15 (m, 1H), 7.12 - 7.06 (m, 1H), 4.69 - 4.59 (m, 1H), 4.49 - 4.40 (m, 1H), 4.15 - 3.94 (m, 3H), 3.92 - 3.72 (m, 3H), 3.54 - 3.45 (m, 2H), 2.26 - 2.13 (m, 4H), 2.10 - 1.96 (m, 1H), 1.90 - 1.46 (m, 12H), 1.43 - 1.34 (m, 1H), 1.16 (d, J = 6.5 Hz, 3H). |
| 2-73 | 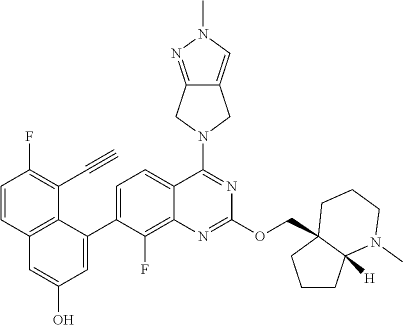 [SMILES: C#Cc1c(F)ccc2cc(O)cc(-c3ccc4c(N5Cc6cn(C)nc6C5)nc(OC[C@]56CCC[C@H]5N(C)CCC6)nc4c3F)c12] | 4-(2-{[(4aS,7aR)-1- methyl-octahydro- 1H- cyclopenta[b]pyridin- 4a-yl]methoxy}-8- fluoro-4-{2-methyl- 2H,4H,5H,6H- pyrrolo[3,4- c]pyrazol-5- yl}quinazolin-7-yl)- 5-ethynyl-6- fluoronaphthalen-2- ol | 621.3 | |
| 2-74 | 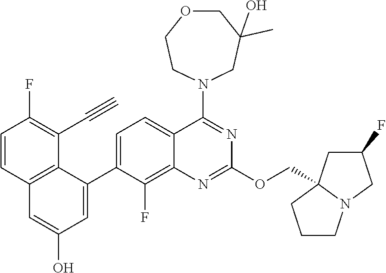 [SMILES: C#Cc1c(F)ccc2cc(O)cc(-c3ccc4c(N5CCOCC(C)(O)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-7-(8- ethynyl-7-fluoro-3- hydroxynaphthalen- 1-yl)-8- fluoroquinazolin-4- yl)-6-methyl-1,4- oxazepan-6-ol | 619.2 | ¹H NMR (400 MHz, DMSO-d₆) δ = 10.14 (s, 1H), 8.04 (d, J = 8.5 Hz, 1H), 7.96 (dd, J = 9.0, 6.3 Hz, 1H), 7.46 (t, J = 9.0 Hz, 1H), 7.36 (d, J = 2.5 Hz, 1H), 7.23 - 7.16 (m, 1H), 7.08 (t, J = 1.9 Hz, 1H), 5.35 - 5.14 (m, 2H), 4.30 - 4.06 (m, 4H), 4.06 - 3.74 (m, 5H), 3.62 - 3.42 (m, 2H), 3.24 - 2.99 (m, 2H), 2.94 - 2.76 (m, 1H), 2.59 - 2.53 (m, 1H), 2.19 - 1.96 (m, 3H), 1.91 - 1.67 (m, 3H), 1.17 - 1.12 (m, 3H). |
| 2-75 | 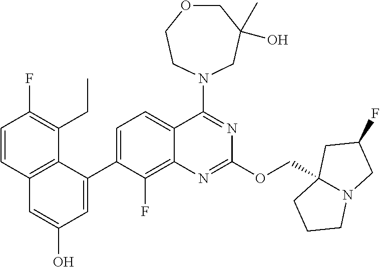 [SMILES: CCc1c(F)ccc2cc(O)cc(-c3ccc4c(N5CCOCC(C)(O)C5)nc(OC[C@@]56CCCN5C[C@H](F)C6)nc4c3F)c12] | 4-(7-(8-Ethyl-7- fluoro-3- hydroxynaphthalen- 1-yl)-8-fluoro-2- (((2R,7aS)-2- fluorotetrahydro-1H- pyrrolizin-7a(5H)- yl)methoxy)quinazolin- 4-yl)-6-methyl- 1,4-oxazepan-6-ol | 623.3 | ¹H NMR (400 MHz, DMSO-d₆) δ ppm = 9.92 (br s, 1H), 8.19 - 8.08 (m, 1H), 7.76 (dd, J = 9.3, 6.0 Hz, 1H), 7.35 (t, J = 9.3 Hz, 1H), 7.32 - 7.24 (m, 2H), 6.93 (d, J = 2.5 Hz, 1H), 5.45 - 5.30 (m, 1H), 5.18 (d, J = 5.0 Hz, 1H), 4.28 - 4.02 (m, 4H), 4.02 - 3.96 (m, 2H), 3.95 - 3.80 (m, 1H), 3.59 - 3.51 (m, 2H), 3.24 - 3.03 (m, 4H), 2.92 - 2.80 (m, 1H), 2.43 - 2.34 (m, 2H), 2.22 - 1.97 (m, 3H), 1.90 - 1.69 (m, 3H), 1.13 (d, J = 16.8 Hz, 3H), 0.80 - 0.66 (m, 3H). |
| 2-76 | 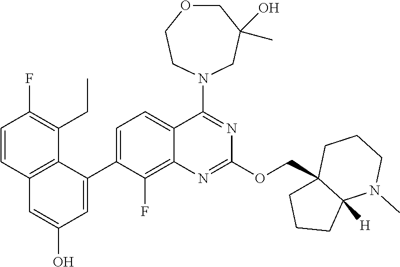 [SMILES: CCc1c(F)ccc2cc(O)cc(-c3ccc4c(N5CCOCC(C)(O)C5)nc(OC[C@]56CCC[C@H]5N(C)CCC6)nc4c3F)c12] | 4-(7-(8-Ethyl-7- fluoro-3- hydroxynaphthalen- 1-yl)-8-fluoro-2- (((4aS,7aR)-1- methyloctahydro- 4aH- cyclopenta[b]pyridin- 4a- yl)methoxy)quinazolin- 4-yl)-6-methyl- 1,4-oxazepan-6-ol | 633.25 | ¹H NMR (400 MHz, DMSO-d₆) δ ppm = 9.96 (br s, 1H), 8.17 - 8.09 (m, 1H), 7.80 - 7.72 (m, 1H), 7.38 - 7.23 (m, 3H), 6.93 (s, 1H), 5.20 (d, J = 4.5 Hz, 1H), 4.46 (d, J = 11.0 Hz, 1H), 4.23 - 3.77 (m, 8H), 3.55 - 3.50 (m, 3H), 2.71 - 2.60 (m, 1H), 2.42 - 2.30 (m, 2H), 2.25 - 2.20 (m, 1H), 2.16 (s, 3H) 1.89 - 1.46 (m, 9H), 1.13 (d, J = 15.0 Hz, 3H), 0.75 - 0.70 (m, 3H). |
| 2-77 | 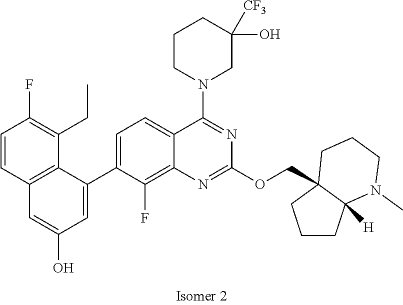 [SMILES: CCc1c(F)ccc2cc(O)cc(-c3ccc4c(N5CCCC(O)(C(F)(F)F)C5)nc(OC[C@]56CCC[C@H]5N(C)CCC6)nc4c3F)c12] | 1-(7-(8-ethyl-7-fluoro-3- hydroxynaphthalen-1- yl)-8-fluoro-2- (((4aS,7aR)-1- methyloctahydro-4aH- cyclopenta[b]pyridin-4a- yl)methoxy)quinazolin- 4-yl)-3- (trifluoromethyl)piperidin- 3-ol | 671.3 | ¹H NMR (400 MHz, DMSO- d₆) δ ppm = 9.93 (br s, 1H), 7.88 (dd, J = 8.6, 4.1 Hz, 1H), 7.76 (dd, J = 9.1, 6.1 Hz, 1H), 7.43 - 7.24 (m, 3H), 6.96 (dd, J = 2.5, 4.8 Hz, 1H), 6.24 (d, J = 8.3 Hz, 1H), 4.55 - 4.50 (m, 1H), 4.41 - 4.21 (m, 2H), 4.11 (dd, J = 11.0, 6.8 Hz, 1H), 3.63 - 3.47 (m, 1H), 3.27 - 3.21 (m, 1H), 2.70 - 2.61 (m, 1H), 2.48 - 2.29 (m, 3H), 2.26 - 2.11 (m, 5H), 1.98 - 1.69 (m, 5H), 1.66 - 1.45 (m, 7H), 1.44 - 1.35 (m, 1H), 0.75 - 0.70 (m, 3H). |
| 2-78 | 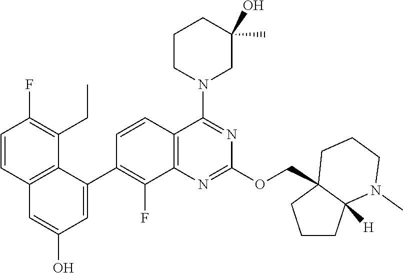 [SMILES: CCc1c(F)ccc2cc(O)cc(-c3ccc4c(N5CCC[C@@](C)(O)C5)nc(OC[C@]56CCC[C@H]5N(C)CCC6)nc4c3F)c12] | (R)-1-(7-(8-ethyl-7- fluoro-3- hydroxynaphthalen-1- yl)-8-fluoro-2- (((4aS,7aR)-1- methyloctahydro-4aH- cyclopenta[b]pyridin-4a- yl)methoxy)quinazolin- 4-yl)-3-methylpiperidin- 3-o1 | 617.3 | ¹H NMR (400 MHz, DMSO- d₆) δ ppm = 9.92 (br s, 1H), 7.95 - 7.89 (m, 1H), 7.76 (dd, J = 9.1, 6.1 Hz, 1H), 7.38 - 7.26 (m, 3H), 6.94 (t, J = 2.7 Hz, 1H), 4.68 (d, J = 8.5 Hz, 1H), 4.48 (t, J = 11.3 Hz, 1H), 4.15 - 4.00 (m, 2H), 3.91 - 3.80 (m, 1H), 3.54 - 3.42 (m, 4H), 2.40 - 2.34 (m, 2H), 2.28 - 2.16 (m, 4H), 2.09 - 1.98 (m, 1H), 1.88 - 1.78 (m, 1H), 1.76 - 1.48 (m, 11H), 1.44 - 1.35 (m, 1H), 1.17 (d, J = 13.1 Hz, 3H), 0.72 (q, J = 7.3 Hz, 3H). |
| 2-79 | 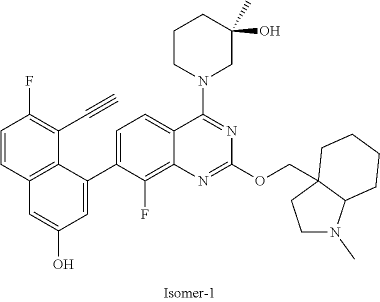 [SMILES: C#Cc1c(F)ccc2cc(O)cc(-c3ccc4c(N5CCC[C@@](C)(O)C5)nc(OCC56CCCCC5N(C)CC6)nc4c3F)c12] | (3R)-1-(7-(8-ethynyl-7- fluoro-3- hydroxynaphthalen-1- yl)-8-fluoro-2-((1- methyloctahydro-3aH- indol-3a- yl)methoxy)quinazolin- 4-yl)-3-methylpiperidin- 3-o1 | 613.3 | ¹H NMR (400 MHz, DMSO- d₆) δ ppm = 10.16 - 9.98 (br s, 1H), 7.96 (dd, J = 9.3, 6.0 Hz, 1H), 7.87 - 7.77 (m, 1H), 7.45 (dt, J = 9.0, 1.3 Hz, 1H), 7.36 (d, J = 2.5 Hz, 1H), 7.13 - 7.02 (m, 1H), 6.80 (dd, J = 11.0, 2.5 Hz, 1H), 4.71 - 4.62 (brs, 1H), 4.41 (dd, J = 10.6, 7.4 Hz, 1H), 4.26 (t, J = 11.3 Hz, 1H), 4.16 - 3.97 (m, 1H), 3.94 - 3.87 (m, 1H), 3.82 (s, 1H), 3.68 (br d, J = 13.3 Hz, 1H), 3.09 - 2.92 (m, 2H), 2.22 - 2.10 (m, 1H), 2.08 (s, 3H), 2.11 - 2.04 (m, 3H), 1.85 - 1.76 (m, 1H), 1.74 - 1.57 (m, 4H), 1.54 - 1.42 (m, 5H), 1.20 - 1.12 (m, 2H), 1.17-1.15 (m, 3H). |
| 2-80 | 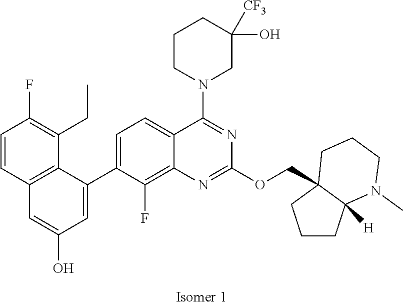 [SMILES: CCc1c(F)ccc2cc(O)cc(-c3ccc4c(N5CCCC(O)(C(F)(F)F)C5)nc(OC[C@]56CCC[C@H]5N(C)CCC6)nc4c3F)c12] | 1-(7-(8-ethyl-7-fluoro-3- hydroxynaphthalen-1- yl)-8-fluoro-2- (((4aS,7aR)-1- methyloctahydro-4aH- cyclopenta[b]pyridin-4a- yl)methoxy)quinazolin- 4-yl)-3- (trifluoromethyl)piperidin- 3-ol | 671.3 | ¹H NMR (400 MHz, DMSO- d₆) δ ppm = 9.91 (s, 1H), 7.88 (dd, J = 8.4, 5.1 Hz, 1H), 7.76 (dd, J = 9.4, 6.1 Hz, 1H), 7.45 - 7.32 (m, 2H), 7.30 (d, J = 2.8 Hz, 1H), 7.05 - 6.89 (m, 1H), 6.25 (d, J = 7.5 Hz, 1H), 4.48 (dd, J = 10.8, 2.5 Hz, 1H), 4.38 - 4.24 (m, 2H), 4.13 (dd, J = 10.8, 5.5 Hz, 1H), 3.61 -3.49 (m, 2H), 3.29 - 3.20 (m, 1H), 2.68 - 2.65 (m, 1H), 2.39 - 2.31 (m, 1H), 2.26 - 2.07 (m, 5H), 1.98 - 1.68 (m, 6H), 1.66 - 1.37 (m, 8H), 0.79 - 0.67 (m, 3H). |
| 2-81 | 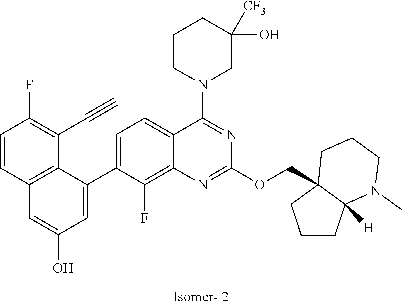 [SMILES: C#Cc1c(F)ccc2cc(O)cc(-c3ccc4c(N5CCCC(O)(C(F)(F)F)C5)nc(OC[C@]56CCC[C@H]5N(C)CCC6)nc4c3F)c12] | 1-(7-(8-ethynyl-7-fluoro- 3-hydroxynaphthalen-1- yl)-8-fluoro-2- (((4aS,7aR)-1- methyloctahydro-4aH- cyclopenta[b]pyridin-4a- yl)methoxy)quinazolin- 4-yl)-3- (trifluoromethyl)piperidin- 3-ol | 667.2 | ¹H NMR (400 MHz, DMSO- d₆) δ ppm = 10.15 (br s, 1H), 7.97 (dd, J = 9.1, 5.9 Hz, 1H), 7.89 - 7.71 (m, 1H), 7.46 (t, J = 9.0 Hz, 1H), 7.37 (dd, J = 2.5, 1.0 Hz, 1H), 7.31 - 7.21 (m, 1H), 7.11 (dd, J = 13.0, 2.5 Hz, 1H), 6.27 (s, 1H), 4.51 - 4.43 (m, 1H), 4.41 - 4.20 (m, 2H), 4.12 (d, J = 10.8 Hz, 1H), 3.83 - 3.67 (m, 1H), 3.61 - 3.48 (m, 1H), 3.23 - 3.15 (m, 2H), 2.80 - 2.71 (m, 1H), 2.36 - 2.23 (m, 5H), 2.01 - 1.49 (m, 12H), 1.47 - 1.35 (m, 1H). |
| 2-82 | 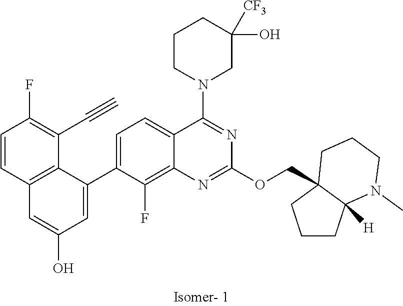 [SMILES: C#Cc1c(F)ccc2cc(O)cc(-c3ccc4c(N5CCCC(O)(C(F)(F)F)C5)nc(OC[C@]56CCC[C@H]5N(C)CCC6)nc4c3F)c12] | 1-(7-(8-ethynyl-7-fluoro- 3-hydroxynaphthalen-1- yl)-8-fluoro-2- (((4aS,7aR)-1- methyloctahydro-4aH- cyclopenta[b]pyridin-4a- yl)methoxy)quinazolin- 4-yl)-3- (trifluoromethyl)piperidin- 3-ol | 667.2 | ¹H NMR (400 MHz, DMSO- d₆) δ ppm = 10.14 (br s, 1H), 7.97 (dd, J = 9.3, 6.0 Hz, 1H), 7.87 - 7.73 (m, 1H), 7.46 (td, J = 9.0, 1.3 Hz, 1H), 7.37 (d, J = 2.5 Hz, 1H), 7.26 (dd, J = 8.3, 7.0 Hz, 1H), 7.11 (dd, J = 13.0, 2.5 Hz, 1H), 6.27 (d, J = 3.8 Hz, 1H), 4.49 - 4.21 (m, 4H), 3.62 - 3.47 (m, 1H), 3.24 (br d, J = 1.5 Hz, 1H), 3.22 - 3.16 (m, 1H), 2.80 - 2.71 (m, 1H), 2.33 - 2.12 (m, 5H), 2.03 - 1.55 (m, 6H), 1.96 - 1.44 (m, 7H), 1.42 - 1.37 (m, 1H). |
| 2-83 | 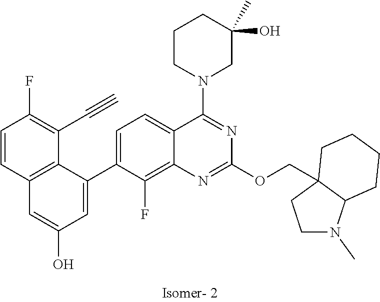 [SMILES: C#Cc1c(F)ccc2cc(O)cc(-c3ccc4c(N5CCC[C@@](C)(O)C5)nc(OCC56CCCCC5N(C)CC6)nc4c3F)c12] | (3R)-1-(7-(8-ethynyl-7- fluoro-3- hydroxynaphthalen-1- yl)-8-fluoro-2-((1- methyloctahydro-3aH- indol-3a- yl)methoxy)quinazolin- 4-yl)-3-methylpiperidin- 3-ol | 613.3 | ¹H NMR (400 MHz, DMSO- d₆) δ ppm = 8.37 (br s, 1H), 7.96 (dd, J = 9.3, 6.0 Hz, 1H), 7.91 - 7.77 (m, 1H), 7.46 (dt, J = 9.0, 1.3 Hz, 1H), 7.36 (d, J = 2.5 Hz, 1H), 7.25 - 7.17 (m, 1H), 7.10 (dd, J = 11.0, 2.5 Hz, 1H), 4.71 - 4.62 (brs, 1H), 4.41 (dd, J = 10.6, 7.4 Hz, 1H), 4.24 (t, J = 11.3 Hz, 1H), 4.16 - 3.97 (m, 1H), 3.94 - 3.87 (m, 1H), 3.82 (s, 1H), 3.78 (br d, J = 13.3 Hz, 1H), 3.09 - 2.99 (m, 2H), 2.26 - 2.14 (m, 1H), 2.08 (s, 3H), 2.11 - 2.04 (m, 3H), 1.85 - 1.76 (m, 1H), 1.74 - 1.57 (m, 4H), 1.54 - 1.42 (m, 5H), 1.20 - 1.12 (m, 2H), 1.17-1.15 (m, 3H). |

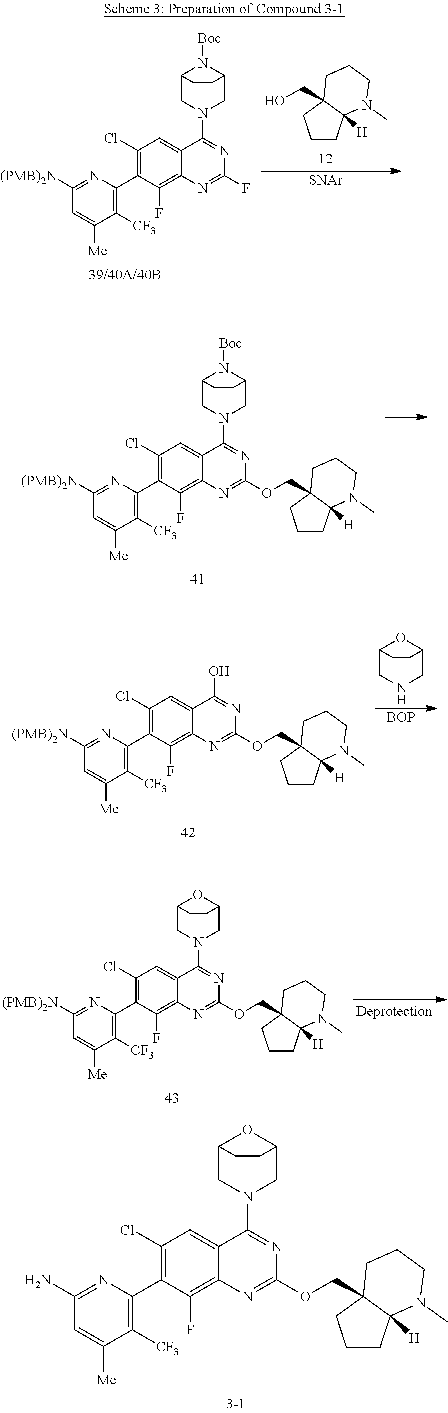

### Preparation of Intermediate 35: tert-butyl 3-(7-bromo-2,6-dichloro-8-fluoroquinazolin-4-yl)-3,8-diazabicyclo[3.2.1]octane-8-carboxylate

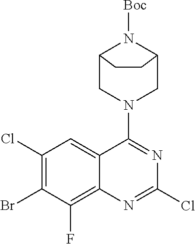 [SMILES: CC(C)(C)OC(=O)N1C2CCC1CN(c1nc(Cl)nc3c(F)c(Br)c(Cl)cc13)C2]

To a solution of 7-bromo-2,4,6-trichloro-8-fluoroquinazoline (300 mg, 3.03 mmol) in dioxane (8 mL) was added DIPEA (0.476 mL, 2.72 mmol) and tert-butyl 3,8-diazabicyclo[3.2.1]octane-8-carboxylate (193 mg, 0.908 mmol). The resulting mixture was stirred at 25° C. for 2 hours. The reaction mixture was concentrated under reduced pressure to provide crude product, which was diluted with ethyl acetate (50 mL) and washed with water (30 mL×2) and brine (50 mL). The organic phase was dried over anhydrous sodium sulfate, filtered, and concentrated under reduced pressure. The residue was purified by COMBIFLASH™ chromatography (Teledyne ISO, Lincoln, NE) (12 g, ISCO column, MeOH/DCM, 0-5%, 20 min.) to provide tert-butyl 3-(7-bromo-2,6-dichloro-8-fluoroquinazolin-4-yl)-3,8-diazabicyclo[3.2.1]octane-8-carboxylate (415 mg, 0.82 mmol, 90% yield) as a white solid. MS (ESI) m/z 507.0 [M+1]⁺. ¹H NMR (499 MHz, DMSO-d₆) δ ppm=8.10 (d, J=1.9 Hz, 1H), 4.38 (br d, J=10.6 Hz, 2H), 4.25 (br s, 2H), 3.66 (m, 2H) 1.79 (m, 2H), 1.62 (m, 2H), 1.47 s, 9H).

### Preparation of Intermediate 36: tert-butyl 3-(7-bromo-6-chloro-2,8-difluoroquinazolin-4-yl)-3,8-diazabicyclo[3.2.1]octane-8-carboxylate

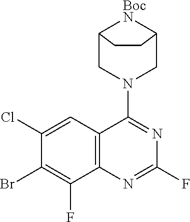 [SMILES: CC(C)(C)OC(=O)N1C2CCC1CN(c1nc(F)nc3c(F)c(Br)c(Cl)cc13)C2]

To a degassed solution of tert-butyl 3-(7-bromo-2,6-dichloro-8-fluoroquinazolin-4-yl)-3,8-diazabicyclo[3.2.1]octane-8-carboxylate (Intermediate 35, 1 g, 3.03 mmol) in DMA (80 mL) was added cesium fluoride (5.25 g, 34.6 mmol). The reaction mixture was degassed with nitrogen for 10 minutes and was heated at 88° C. for 5 h in a sealed tube. Water (200 mL) and ethyl acetate (150 mL) were added, and the reaction mixture was stirred for 15 minutes. The separated aqueous layer was extracted with ethyl acetate (2×100 mL) and the combined organic layers were dried over anhydrous Na₂SO₄, filtered, and concentrated under reduced pressure. The resulting residue was purified by COMBIFLASH™ chromatography (Teledyne ISO, Lincoln, NE) with 15-25% ethyl acetate in petroleum ether as an eluent to provide tert-butyl-3-(7-bromo-6-chloro-2,8-difluoroquinazolin-4-yl)-3,8-diazabicyclo[3.2.1]octane-8-carboxylate (4.7 g, 8.77 mmol, 63.4% yield) as a pale-yellow solid. MS (ESI) m/z 489.0 [M+1]⁺.

### Preparation of Intermediate 37: tert-butyl 3-(7-(6-(bis(4-methoxybenzyl)amino)-4-methylpyridin-2-yl)-6-chloro-2,8-difluoroquinazolin-4-yl)-3,8-diazabicyclo[3.2.1]octane-8-carboxylate

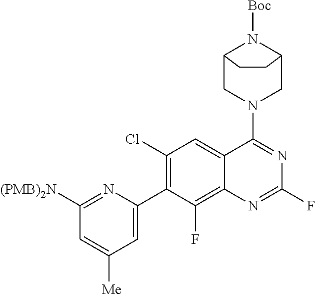

To a degassed solution of tert-butyl-3-(7-bromo-6-chloro-2,8-difluoroquinazolin-4-yl)-3,8-diazabicyclo[3.2.1]octane-8-carboxylate (Intermediate 36, 2.00 g, 4.08 mmol) in anhydrous 1,4-dioxane (20 mL) were added potassium phosphate (1.73 g, 8.17 mmol), N,N-bis(4-methoxybenzyl)-4-methyl-6-(4,4,5,5-tetramethyl-1,3,2-dioxaborolan-2-yl)pyridin-2-amine (5.8 g, 12.25 mmol) and PdCl₂(dppf) (149 mg, 0.204 mmol). The reaction mixture was degassed again and heated at 80° C. for 48 h. After completion of the reaction, the reaction mixture was allowed to cool to ambient temperature, diluted with ethyl acetate (40 mL), filtered through a bed of a diatomaceous earth (CELITE™, Sigma Aldrich, St. Louis, MO) and concentrated under reduced pressure to provide crude product. The residue was purified by COMBIFLASH™ chromatography (Teledyne ISO, Lincoln, NE) using 30% ethyl acetate in petroleum ether to obtain tert-butyl 3-(7-(6-(bis(4-methoxybenzyl)amino)-4-methylpyridin-2-yl)-6-chloro-2,8-difluoroquinazolin-4-yl)-3,8-diazabicyclo[3.2.1]octane-8-carboxylate (1.5 g, 1.74 mmol, 42% yield). ¹H NMR (400 MHz, CDCl₃): δ ppm=7.76 (d, J=1.6 Hz, 1H), 7.20-7.18 (d, J=8.8 Hz, 4H), 6.86 (dt, J=9.6 Hz, 4H), 6.60 (s, 1H), 6.38 (s, 1H), 4.60 (s, 3H), 4.39-4.21 (m, 4H), 3.63 (s, 6H), 2.29 (s, 3H), 1.98-1.96 (m, 6H), 1.76-1.63 (m, 2H), 1.49 (s, 9H) LCMS (ESI) m/z: 757.2 [M+H]⁺.

### Preparation of Intermediate 38: tert-butyl 3-(7-(6-(bis(4-methoxybenzyl)amino)-3-iodo-4-methylpyridin-2-yl)-6-chloro-2,8-difluoroquinazolin-4-yl)-3,8 diazabicyclo[3.2.1]octane-8-carboxylate

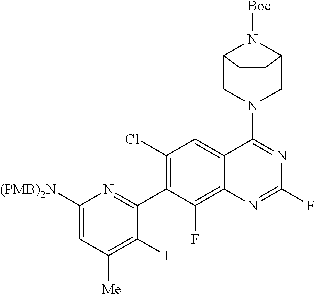

To a stirred solution of tert-butyl 3-(7-(6-(bis(4-methoxybenzyl)amino)-4-methylpyridin-2-yl)-6-chloro-2,8-difluoroquinazolin-4-yl)-3,8-diazabicyclo[3.2.1]octane-8-carboxylate (Intermediate 37, 1.40 g, 1.849 mmol) in anhydrous ACN (15 mL) under nitrogen at 0° C. were added N-iodosuccinimide (0.42 g, 1.849 mmol) and trifluoroacetic acid (0.028 mL, 0.370 mmol). The reaction mixture was allowed to reach room temperature over one hour. The reaction mixture was then quenched with saturated aqueous sodium thiosulfate (5 mL) and saturated aqueous sodium bicarbonate (4 mL). The mixture was extracted with ethyl acetate (3×20 mL). Combined organic layers were dried over anhydrous sodium sulfate, filtered, and concentrated under vacuum to obtain the crude residue. The crude residue was purified by COMBIFLASH™ chromatography (Teledyne ISO, Lincoln, NE) using 30% ethyl acetate in petroleum ether to obtain tert-butyl 3-(7-(6-(bis(4-methoxybenzyl)amino)-3-iodo-4-methylpyridin-2-yl)-6-chloro-2,8-difluoroquinazolin-4-yl)-3,8 diazabicyclo[3.2.1]octane-8-carboxylate (1.42 g, 1.560 mmol, 84% yield) as pale yellow fluffy solid. LCMS (ESI) m/z: 883.3 [M+H]⁺.

### Preparation of Intermediate 39: tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate

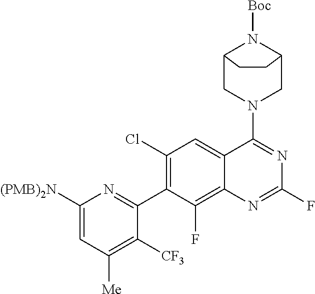

To a stirred solution of tert-butyl 3-(7-(6-(bis(4-methoxybenzyl)amino)-3-iodo-4-methylpyridin-2-yl)-6-chloro-2,8-difluoroquinazolin-4-yl)-3,8-diazabicyclo[3.2.1]octane-8-carboxylate (Intermediate 38, 1.40 g, 1.585 mmol) in anhydrous DMA (10 mL) in a sealed tube under nitrogen was added copper(I) iodide (0.60 g, 3.17 mmol). The reaction mixture was degassed 10 minutes before the addition of methyl 2,2-difluoro-2-(fluorosulfonyl)acetate (0.91 g, 4.76 mmol) and reaction mixture was heated at 90° C. for 12 h. Reaction progress was monitored by LCMS. Reaction mixture was diluted with diethyl ether (20 mL) and water (10 mL). Layers were separated and aqueous layer was extracted with diethyl ether (3×20 mL). Combined organic layers were dried over anhydrous sodium sulfate, filtered, and concentrated under reduced pressure to obtain the crude residue. The crude compound was purified by COMBIFLASH™ chromatography (Teledyne ISO, Lincoln, NE) using 30% ethyl acetate in petroleum ether to obtain tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate (0.85 g, 0.630 mmol, 40% yield) as a pale-yellow solid. ¹H NMR (400 MHz, CDCl₃): δ ppm=7.77 (s, 1H), 7.16 (d, J=8.8 Hz, 4H), 6.87 (dt, J=9.6 and 2.8 Hz, 4H), 6.43 (s, 1H), 4.76-4.72 (m, 2H), 4.59-4.55 (m, 2H), 3.81 (s, 6H), 2.43 (s, 3H), 1.97-1.82 (m, 4H), 1.97-1.82 (m, 4H), 1.53 (s, 9H). LCMS (ESI) m/z: 825.2 [M+H]⁺.

### Intermediates 40A and 40B: tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate

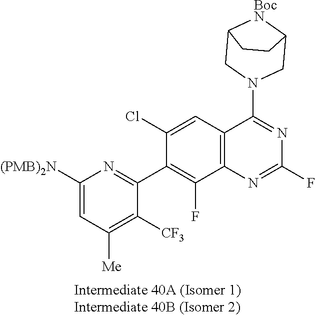

Intermediate 39 (5.0 g, 6.06 mmol) was subjected to SFC separation (Column: CHIRALPAK™ IH (Daicel, Japan) (250 mm×4.6 mm; 5 μm), mobile phase: 0.25% Isopropanol), where Peak-1 eluted at retention time=5.85 min (2.4 g, 2.90 mmol, 40% yield) and Peak-2 at retention time=9.53 min (2.4 g, 2.90 mmol, 40% yield). Peak-1 (40A): ¹H NMR (400 MHz, CDCl₃): δ ppm=7.78 (s, 1H), 7.16 (d, J=8.8 Hz, 4H), 6.87 (dt, J=9.6 and 2.8 Hz, 4H), 6.43 (s, 1H), 4.76-4.72 (m, 2H), 4.59-4.55 (m, 2H), 3.81 (s, 6H), 2.43 (s, 3H), 1.97-1.82 (m, 4H), 1.97-1.82 (m, 4H), 1.53 (s, 9H) ppm. LCMS (ESI) m/z: 825.2 [M+H]⁺. LCMS (ESI) m/z: 825.2 [M+H]⁺. [α]^{23.5} (MeOH=0.10)=+96.00; Peak-2: (40B)¹H NMR (400 MHz, CDCl₃): δ ppm=7.78 (s, 1H), 7.16 (d, J=8.8 Hz, 4H), 6.87 (dt, J=9.6 and 2.8 Hz, 4H), 6.43 (s, 1H), 4.76-4.72 (m, 2H), 4.59-4.55 (m, 2H), 3.81 (s, 6H), 2.43 (s, 3H), 1.97-1.82 (m, 4H), 1.97-1.82 (m, 4H), 1.53 (s, 9H) ppm. LCMS (ESI) m/z: 825.2 [M+H]⁺. [α]^{23.3} (MeOH=0.10)=−110.00.

### Preparation of Intermediate 41: tert-butyl 3-(2-{[(4aS,7aR)-1-methyl-octahydro-1H-cyclopenta[b]pyridin-4a-yl]methoxy}-7-(6-{bis[(4-methoxyphenyl)methyl]amino}-4-methyl-3-(trifluoromethyl)pyridin-2-yl)-6-chloro-8-fluoroquinazolin-4-yl)-3,8-diazabicyclo[3.2.1]octane-8-carboxylate

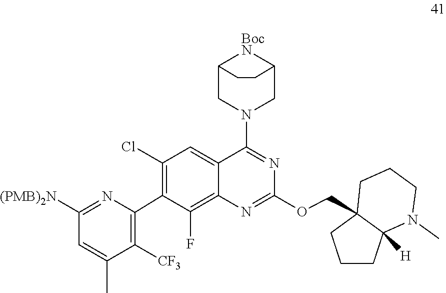

To a solution of [(4aS,7aR)-1-methyl-octahydro-1H-cyclopenta[b]pyridin-4a-yl]methanol (Intermediate 40B, 0.205 g, 1.212 mmol) in THE (5 mL) at 0° C. was added NaH (0.194 g, 4.85 mmol) and the mixture was stirred at room temperature for 30 minutes. To this was added tert-butyl (1R,5S)-3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate (1 g, 1.212 mmol) at 0° C. and mixture was stirred at room temperature for 1 h. The reaction mixture was quenched with ice cubes and concentrated under reduced pressure. The crude was partitioned in between ethyl acetate and water. Organic layer was dried over sodium sulfate, filtered, and concentrated to provide the crude which was purified by COMBIFLASH™ chromatography (Teledyne ISO, Lincoln, NE) (24 g REDISEP™ column (Teledyne ISCO, Lincoln, NE), 0 to 10% MeOH-DCM) to provide tert-butyl 3-(2-{[(4aS,7aR)-1-methyl-octahydro-1H-cyclopenta[b]pyridin-4a-yl]methoxy}-7-(6-{bis[(4-methoxyphenyl)methyl]amino}-4-methyl-3-(trifluoromethyl)pyridin-2-yl)-6-chloro-8-fluoroquinazolin-4-yl)-3,8-diazabicyclo[3.2.1]octane-8-carboxylate (0.70 g, 0.718 mmol, 59.3% yield). MS(ESI) m/z: 974.4 (M+H)⁺.

### Preparation of Intermediate 42: 2-{[(4aS,7aR)-1-methyl-octahydro-1H-cyclopenta[b]pyridin-4a-yl]methoxy}-7-(6-{bis[(4-methoxyphenyl)methyl]amino}-4-methyl-3-(trifluoromethyl)pyridin-2-yl)-6-chloro-8-fluoroquinazolin-4-ol

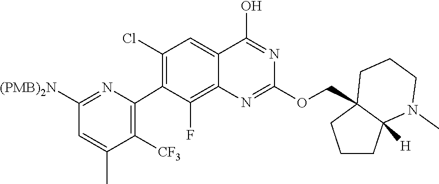

To a stirred solution of tert-butyl 3-(2-{[(4aS,7aR)-1-methyl-octahydro-1H-cyclopenta[b]pyridin-4a-yl]methoxy}-7-(6-{bis[(4-methoxyphenyl)methyl]amino}-4-methyl-3-(trifluoromethyl)pyridin-2-yl)-6-chloro-8-fluoroquinazolin-4-yl)-3,8-diazabicyclo[3.2.1]octane-8-carboxylate (Intermediate 41, 300 mg, 0.308 mmol) in EtOH (4 mL), THE (4 mL) was added sodium hydroxide (1M aq. soln.) (3.08 mL, 3.08 mmol). The reaction mixture was heated at 70° C. for 18h. The reaction mixture was evaporated, and the residue was diluted with ethyl acetate. The organic layer was washed with saturated NH₄Cl solution, water, brine, dried over anhydrous sodium sulfate, filtered, and concentrated under reduced pressure to provide the crude compound, which was purified by COMBFLASH™ chromatography (Teledyne ISO, Lincoln, NE) (24 g silica gel column, using 10-20% MeOH/DCM) to provide 2-{[(4aS,7aR)-1-methyl-octahydro-1H-cyclopenta[b]pyridin-4a-yl]methoxy}-7-(6-{bis[(4 methoxyphenyl)methyl]amino}-4-methyl-3-(trifluoromethyl)pyridin-2-yl)-6-chloro-8-fluoroquinazolin-4-ol (110 mg, 0.141 mmol, 45.8% yield) as a brown solid. MS(ESI) m/z: 780.5 [M+H]⁺.

### Preparation of Intermediate 43: 6-(2-{[(4aS,7aR)-1-methyl-octahydro-1H-cyclopenta[b]pyridin-4a-yl]methoxy}-6-chloro-8-fluoro-4-{8-oxa-3-azabicyclo[3.2.1]octan-3-yl}quinazolin-7-yl)-N,N-bis[(4-methoxyphenyl)methyl]-4-methyl-5-(trifluoromethyl)pyridin-2-amine

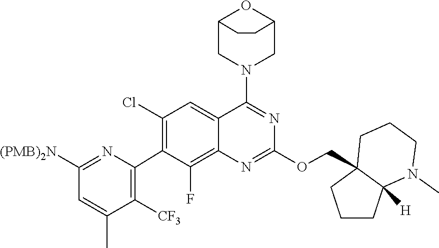

To a stirred solution of 2-{[(4aS,7aR)-1-methyl-octahydro-1H-cyclopenta[b]pyridin-4a-yl]methoxy}-7-(6-{bis[(4-methoxyphenyl)methyl]amino}-4-methyl-3-(trifluoromethyl)pyridin-2-yl)-6-chloro-8-fluoroquinazolin-4-ol (Intermediate 42, 20 mg, 0.026 mmol) in ACN (1 mL) was added (1R,5S)-8-oxa-3-azabicyclo[3.2.1]octane (2.90 mg, 0.026 mmol), DIPEA (0.013 mL, 0.077 mmol) and BOP (17.01 mg, 0.038 mmol). The reaction mixture was heated at 80° C. for 8 h. The reaction mixture was evaporated, and the residue was diluted with ethyl acetate. The organic layer was washed with water, brine, dried over anhydrous sodium sulfate, filtered, and concentrated under reduced pressure to provide the crude 6-(2-{[(4aS,7aR)-1-methyl-octahydro-1H-cyclopenta[b]pyridin-4a-yl]methoxy}-6-chloro-8-fluoro-4-{8-oxa-3-azabicyclo[3.2.1]octan-3-yl}quinazolin-7-yl)-N,N-bis[(4-methoxyphenyl)methyl]-4-methyl-5-(trifluoromethyl)pyridin-2-amine (20 mg, 0.023 mmol, 89% yield) as a brown liquid, which was taken for next step without purification. MS(ESI) m/z: 875.3 [M+H]⁺.

### Example 3-1:6-(2-{[(4aS,7aR)-1-methyl-octahydro-1H-cyclopenta[b]pyridin-4a-yl]methoxy}-6-chloro-8 fluoro-4-{8-oxa-3-azabicyclo[3.2.1]octan-3-yl}quinazolin-7-yl)-4-methyl-5-(trifluoromethyl)pyridin-2-amine

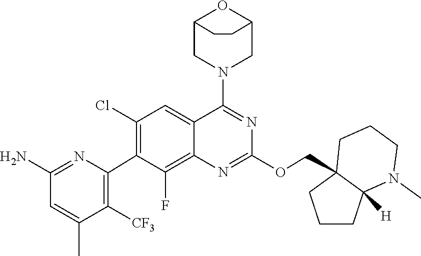 [SMILES: Cc1cc(N)nc(-c2c(Cl)cc3c(N4CC5CCC(C4)O5)nc(OC[C@]45CCC[C@H]4N(C)CCC5)nc3c2F)c1C(F)(F)F]

To a stirred solution of 6-(4-((1R,5S)-8-oxa-3-azabicyclo[3.2.1]octan-3-yl)-6-chloro-8-fluoro-2-(((4aS,7aR)-1-methyloctahydro-4aH-cyclopenta[b]pyridin-4a-yl)methoxy)quinazolin-7-yl)-N,N-bis(4-methoxybenzyl)-4-methyl-5-(trifluoromethyl)pyridin-2-amine (Intermediate 43, 20 mg, 0.023 mmol) in TFA (35.2 μl, 0.457 mmol) at 0° C. was added triethylsilane (10.95 μl, 0.069 mmol) and the reaction mixture was stirred at 40° C. for 16 h. The volatiles from the reaction mixture were removed under reduced pressure (at lower temperature, ˜30° C.) and the crude residue was co-evaporated with 1,4-dioxane. Then the crude residue was dissolved in 1,4-dioxane and added triethylamine and evaporated under reduced pressure to provide an off-white solid. The crude material was purified via preparative LC/MS with the following conditions: Column: Waters XBridge C18, 19×150 mm, 5-μm particles; Mobile Phase A: 0.1% trifluoroacetic acid; Mobile Phase B: acetonitrile; Gradient: 15-42% B over 23 minutes, then a 5-minute. hold at 100% B; Flow: 20 mL/min to provide 6-(2-{[(4aS,7aR)-1-methyl-octahydro-1H-cyclopenta[b]pyridin-4a-yl]methoxy}-6-chloro-8-fluoro-4-{8-oxa-3-azabicyclo[3.2.1]octan-3-yl}quinazolin-7-yl)-4-methyl-5-(trifluoromethyl)pyridin-2-amine (6 mg, 8.97 μmol, 39.3% yield)): MS(ESI) m/z: 635.3, [M+H]⁺. ¹H NMR (400 MHz, DMSO-d₆) δ ppm=9.66-8.76 (m, 1H), 7.86 (s, 1H), 6.83 (br s, 2H), 6.50 (s, 1H), 4.60-4.22 (m, 5H), 4.20-4.06 (m, 1H), 3.80-3.52 (m, 2H), 3.17-3.03 (m, 1H), 2.88-2.69 (m, 3H), 2.37 (d, J=1.3 Hz, 3H), 2.05-1.91 (m, 1H), 1.88-1.43 (m, 12H).

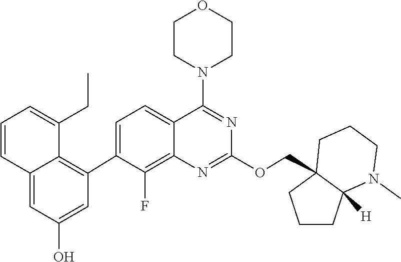 [SMILES: CCc1cccc2cc(O)cc(-c3ccc4c(N5CCOCC5)nc(OC[C@]56CCC[C@H]5N(C)CCC6)nc4c3F)c12]

### Example 4-1: 4-(2-{[(4aS,7aR)-1-methyl-octahydro-1H-cyclopenta[b]pyridin-4a-yl]methoxy}-8-fluoro-4-(morpholin-4-yl)quinazolin-7-yl)-5-ethylnaphthalen-2-ol

### Preparation of Intermediate 44: 8-((triisopropylsilyl)ethynyl)naphthalene-1,3-diol

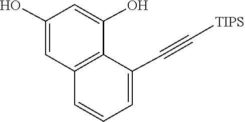

To a stirred solution of naphthalene-1,3-diol (20 g, 125 mmol), bromoethynyl)triisopropylsilane (34.3 g, 131 mmol), and potassium acetate (24.51 g, 250 mmol) in anhydrous 1,4-dioxane (200 mL) was added dichloro(pcymene)ruthenium(II) dimer (7.65 g, 12.49 mmol) under a nitrogen atmosphere. The resulting mixture was stirred for 12h at 110° C. The reaction mixture was cooled to ambient temperature and was filtered through diatomaceous earth (CELITE™, Sigma Aldrich, St. Louis, MO). The bed was washed with EtOAc (2×100 mL), the filtrate was combined and concentrated reduced pressure to obtain a crude residue. The crude material was purified by flash column (silica 100-200 mesh) chromatography using 12-15% ethyl acetate in petroleum ether as eluent. Pure fractions were concentrated under reduced pressure to obtain 8-((triisopropylsilyl)ethynyl)naphthalene-1,3-diol (30 g, 85 mmol, 67.7% yield). LCMS (ESI) m/z: 341.55 [M+H]⁺.

### Preparation of Intermediate 45: 3-(methoxymethoxy)-8-((triisopropylsilyl)ethynyl)naphthalen-1-ol

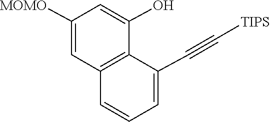

To a stirred solution of 8-((triisopropylsilyl)ethynyl)naphthalene-1,3-diol (Intermediate 44, 25 g, 73.4 mmol) in anhydrous DCM (300 mL) under nitrogen was added DIPEA (38.5 mL, 220 mmol) at ˜10° C. After 10 min, MOM-Cl (6.13 mL, 81 mmol) was added to the reaction mixture dropwise over 20 min under a nitrogen atmosphere. The resulting mixture was stirred for 2h at the same temperature. The reaction mixture was diluted with 100 mL of DCM and washed with 200 mL of brine. The combined organics were dried over anhydrous Na₂SO₄, filtered, and concentrated reduced pressure to yield a crude residue. The crude material was purified by flash column (silica 100-200 mesh) chromatography using 5-10% ethyl acetate in petroleum ether as eluent. Pure fractions were concentrated under reduced pressure to obtain 3-(methoxymethoxy)-8-((triisopropylsilyl)ethynyl)naphthalen-1-ol (21 g, 53.5 mmol, 72.9% yield) as a pale yellow oil. LCMS (ESI) m/z: 385.60 [M+H]⁺.

### Preparation of Intermediate 46: 3-(methoxymethoxy)-8-((triisopropylsilyl)ethynyl)naphthalen-1-yl pivalate

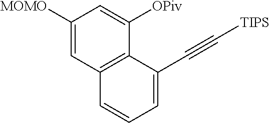

To a stirred solution of 3-(methoxymethoxy)-8-((triisopropylsilyl)ethynyl)naphthalen-1-ol (Intermediate 45, 20 g, 52.0 mmol), TEA (21.75 mL, 156 mmol), DMAP (1.271 g, 10.40 mmol) in anhydrous DCM (200 mL) at −10° C. under a nitrogen atmosphere was added pivaloyl chloride (12.80 mL, 104 mmol) dropwise for 10 min. The resulting mixture was stirred for 2 h at room temperature under nitrogen. The reaction mixture was then diluted with 200 mL of DCM and washed with brine (200 mL). The organics were dried over anhydrous Na₂SO₄, filtered, and concentrated reduced pressure to yield a crude residue. The crude was purified by flash column (silica 100-200 mesh) chromatography using 10-15% ethyl acetate in petroleum ether as eluent. Pure fractions were concentrated under reduced pressure to obtain 3-(methoxymethoxy)-8-((triisopropylsilyl)ethynyl)naphthalen-1-yl pivalate (23 g, 49.1 mmol, 94% yield) as a pale yellow oil. LCMS (ESI) m/z: 469.2 [M+H]⁺.

### Preparation of Intermediate 47: 8-ethynyl-3-(methoxymethoxy)naphthalen-1-yl pivalate

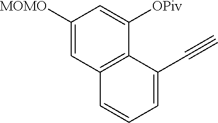

To a stirred solution of 3-(methoxymethoxy)-8-((triisopropylsilyl)ethynyl)naphthalen-1-yl pivalate (Intermediate 46, 12 g, 25.6 mmol) in anhydrous DMF (130 mL) at room temperature under a nitrogen atmosphere was added anhydrous CsF (27.2 g, 179 mmol). The reaction mixture was stirred for 2 h. The reaction mixture was diluted with DCM (200 mL) and washed with brine (200 mL). The organics were dried over anhydrous Na₂SO₄, filtered, and concentrated reduced pressure to yield a crude residue. The crude was purified by flash column (silica 100-200 mesh) chromatography using 5-8% ethyl acetate in petroleum ether as eluent. Pure fractions were concentrated under reduced pressure to obtain 8-ethynyl-3-(methoxymethoxy)naphthalen-1-yl pivalate(7 g, 20.62 mmol, 81% yield). LCMS (ESI) m/z: 313.3 [M+H]⁺.

### Preparation of Intermediate 48: 8-ethyl-3-methoxymethoxy)naphthalen-1-ylpivalate

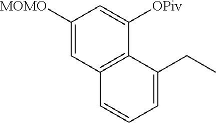

To a stirred solution of 8-ethynyl-3-(methoxymethoxy)naphthalen-1-yl pivalate (Intermediate 47, 7 g, 22.41 mmol) in anhydrous methanol (70 mL) was added Pd/C (1.4 g, 13.16 mmol) at 25° C. The suspension was degassed reduced pressure and purged with H2 several times. The mixture was stirred under H2 atmosphere (1 atm) at 25° C. for 5 hours. The reaction mixture was filtered, and concentrated under reduced pressure to obtain a crude residue. The crude material was purified by flash column (silica 100-200 mesh) chromatography using 15-20% ethyl acetate in petroleum ether as eluent. Pure fractions were concentrated under reduced pressure to provide 8-ethyl-3-methoxymethoxy)naphthalen-1-ylpivalate (6.56 g, 17.83 mmol, 80% yield) as pale yellow oil. LCMS (ESI) m/z: 316.1 [M+H]⁺.

### Preparation of Intermediate 49: 8-ethyl-3-methoxymethoxy)naphthalen-1-ol

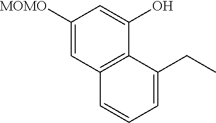

To a stirred solution of 8-ethyl-3-(methoxymethoxy)naphthalen-1-yl pivalate (Intermediate 48, 10 g, 31.6 mmol) in THF:Water:MeOH (5:1:5) was added anhydrous LiOH (1.135 g, 47.4 mmol) at room temperature under nitrogen. The resulting mixture was stirred for 3 h at the same temperature. The reaction mixture was concentrated reduced pressure to remove the MeOH. The reaction mixture was diluted with EtOAc (150 mL) and washed with brine (200 mL). The organics were dried over anhydrous Na₂SO₄, filtered, and concentrated reduced pressure to yield a crude residue. The crude material was purified by flash column (silica 100-200 mesh) chromatography using 25-30% ethyl acetate in petroleum ether as eluent. Pure fractions were concentrated under reduced pressure to obtain 8-ethyl-3-methoxymethoxy)naphthalen-1-ol (6 g, 25.8 mmol, 82% yield). LCMS (ESI) m/z: 233.2 [M+H]⁺.

### Preparation of Intermediate 50: 8-ethyl-3-(methoxymethoxy)naphthalen-1-yl trifluoromethanesulfonate

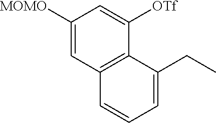

To a stirred solution of 8-ethyl-3-(methoxymethoxy)naphthalen-1-ol (Intermediate 49, 3 g, 12.92 mmol) and DIPEA (22.50 mL, 129 mmol) in anhydrous dichloromethane (50 mL) at −40° C. under nitrogen was added Tf₂O (2.182 mL, 12.92 mmol) dropwise. The resultant reaction mixture was allowed to warm to room temperature and stirred for 1 h. The reaction mixture was diluted with cold water (500 mL) and extracted with DCM (2×500 mL). The combined organic layers were washed with brine (50 mL), dried over anhydrous Na₂SO₄, filtered, and concentrated under reduced pressure to obtain a crude residue. The crude compound was purified by flash column (Silicagel 100-200) chromatography using 5-10% ethyl acetate in petroleum ether as eluent. Pure fractions were concentrated under reduced pressure to provide 8-ethyl-3-(methoxymethoxy)naphthalen-1-yl trifluoromethanesulfonate (3.5 g, 9.03 mmol, 69.9% yield) as a pale yellow oil. LCMS (ESI) m/z: 365.3 [M+H]⁺.

### Preparation of Intermediate 51: 2-(8-ethyl-3-(methoxymethoxy)naphthalen-1-yl)-4,4,5,5-tetramethyl-1,3,2-dioxaborolane

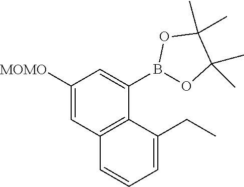

To a stirred solution of 8-ethyl-3-(methoxymethoxy)naphthalen-1-yl trifluoromethanesulfonate (Intermediate 50, 7.4 g, 20.31 mmol) in 1,4-dioxane (80 mL) was added bispinacalatoborane (12.89 g, 50.8 mmol) and potassium acetate (5.98 g, 60.9 mmol). The mixture was degassed and purged with nitrogen for 5 min and PdCl₂(dppf) (1.659 g, 2.031 mmol) was added. The resulting mixture was stirred for 3 h at 100° C. temperature under nitrogen. The reaction mixture was diluted with water (50 mL) and extracted with ethyl acetate (250 mL). The combined organic layers were washed with brine (50 mL) dried over anhydrous Na₂SO₄ and concentrated under reduced pressure to obtain a crude residue. The crude was purified by flash column (silica 100-200 mesh) chromatography by using 2-4% ethyl acetate in petroleum ether as eluent. Pure fractions were concentrated under reduced pressure to provide a brown gum which was again purified by reverse phase column chromatography using 80% Acetonitrile in 0.01% of Ammonium formate in water as eluent. Pure fractions were concentrated reduced pressure to obtain 2-(8-ethyl-3-(methoxymethoxy)naphthalen-1-yl)-4,4,5,5-tetramethyl-1,3,2-dioxaborolane (4.5 g, 13.10 mmol, 64.5% yield). LCMS (ESI) m/z: 344.2 [M+H]⁺. ¹H-NMR (400 MHz, CDCl₃): δ 7.62 (dd, J=0.80, 8.00 Hz, 1H), 7.36-7.44 (m, 3H), 7.27 (t, J=0.40 Hz, 1H), 5.31 (s, 2H), 3.53 (s, 3H), 3.21 (q, J=7.20 Hz, 2H), 1.46 (s, 12H), 1.38 (t, J=7.60 Hz, 3H) ppm.

### Preparation of Intermediate 52: tert-butyl (1R,5S)-3-(7-(8-ethyl-3-(methoxymethoxy)naphthalen-1-yl)-8-fluoro-2-(((4aS,7aR)-1-methyloctahydro-4aH-cyclopenta[b]pyridin-4a-yl)methoxy)quinazolin-4-yl)-3,8-diazabicyclo[3.2.1]octane-8-carboxylate

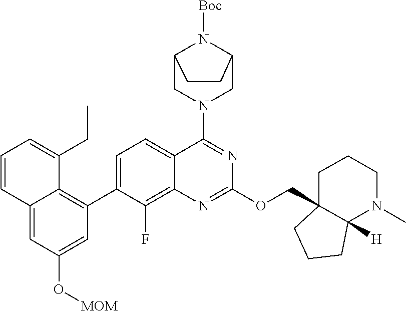

A mixture of Intermediate 30 (550 mg, 0.910 mmol), 2-(8-ethyl-3-(methoxymethoxy)naphthalen-1-yl)-4,4,5,5-tetramethyl-1,3,2-dioxaborolane (327 mg, 0.955 mmol), [1,1′-bis(di-tert-butylphosphino)ferrocene]dichloropalladium(II) (29.6 mg, 0.045 mmol) and 2.0 M Potassium phosphate tribasic (1365 μl, 2.73 mmol) in dioxane (1 mL) under nitrogen was stirred at 50° C. for 18 hours. To the mixture was added EtOAc (25 mL) and the ethyl acetate layer was dried over sodium sulfate, filtered and concentrated. The crude product was purified by prep-HPLC (ISCO C18 100 g column, flow rate=60 ml/min., gradient=40% A to 100% B in 20 min., A=H₂O/ACN/TFA (90:10:0.1), B=H₂O/ACN/TFA (10:90:0.1)). The pure fractions were combined. and concentrated. The mixture was diluted with EtOAc (25 mL) and was washed with a solution of aqueous saturated sodium carbonate (25 mL). The ethyl acetate layer was dried over sodium sulfate, filtered, and concentrated to provide tert-butyl (1R,5S)-3-(7-(8-ethyl-3-(methoxymethoxy)naphthalen-1-yl)-8-fluoro-2-(((4aS,7aR)-1-methyloctahydro-4aH-cyclopenta[b]pyridin-4a-yl)methoxy)quinazolin-4-yl)-3,8-diazabicyclo[3.2.1]octane-8-carboxylate (550 mg, 0.706 mmol, 78% yield) as tan foam. MS(ESI) m/z: 740.3 [M+H]⁺.

### Preparation of Intermediate 53: 7-(8-ethyl-3-(methoxymethoxy)naphthalen-1-yl)-8-fluoro-2-(((4aS,7aR)-1-methyloctahydro-4aH-cyclopenta[b]pyridin-4a-yl)methoxy)quinazolin-4-ol

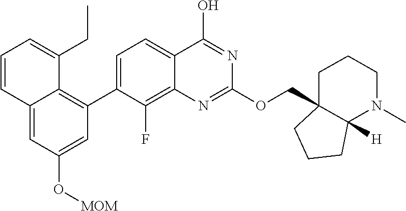

A mixture of tert-butyl (1R,5S)-3-(7-(8-ethyl-3-(methoxymethoxy)naphthalen-1-yl)-8-fluoro-2-(((4aS,7aR)-1-methyloctahydro-4aH-cyclopenta[b]pyridin-4a-yl)methoxy)quinazolin-4-yl)-3,8-diazabicyclo[3.2.1]octane-8-carboxylate (Intermediate 52, 550 mg, 0.743 mmol) and 1.0 M sodium hydroxide (7433 μl, 7.43 mmol) and EtOH (25 mL) was stirred at 60° C. for 3 days. The mixture was concentrated. The mixture was extracted with EtOAc (25 mL) and the ethyl acetate layer was dried over sodium sulfate, filtered, and concentrated. The crude product was purified by prep-HPLC (ISCO C18 100 g column, flow rate=60 ml/min., gradient=20% A to 100% B in 20 min., A=H₂O/ACN/TFA (90:10:0.1), B=H₂O/ACN/TFA (10:90:0.1)). The pure fractions were combined and concentrated. The mixture was diluted with EtOAc (15 mL) and was washed with a solution of aqueous saturated sodium carbonate (15 mL). The ethyl acetate layer was dried over sodium sulfate, filtered, and concentrated to provide 7-(8-ethyl-3-(methoxymethoxy)naphthalen-1-yl)-8-fluoro-2-(((4aS,7aR)-1-methyloctahydro-4aH-cyclopenta[b]pyridin-4a-yl)methoxy)quinazolin-4-ol (287 mg, 0.500 mmol, 67.2% yield) as white solid. MS(ESI) m/z: 546.4 [M+H]⁺.

### Example 4-1: 4-(2-{[(4aS,7aR)-1-methyl-octahydro-1H-cyclopenta[b]pyridin-4a-yl]methoxy}-8-fluoro-4-(morpholin-4-yl)quinazolin-7-yl)-5-ethylnaphthalen-2-ol

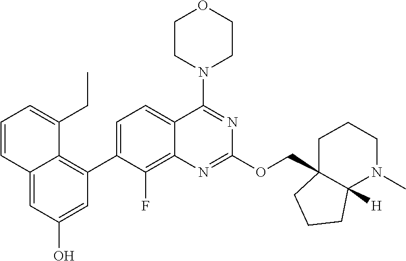 [SMILES: CCc1cccc2cc(O)cc(-c3ccc4c(N5CCOCC5)nc(OC[C@]56CCC[C@H]5N(C)CCC6)nc4c3F)c12]

To a solution of 7-(8-ethyl-3-(methoxymethoxy)naphthalen-1-yl)-8-fluoro-2-(((4aS,7aR)-1-methyloctahydro-4aH-cyclopenta[b]pyridin-4a-yl)methoxy)quinazolin-4-ol (Intermediate 52, 20 mg, 0.037 mmol), TEA (25.5 mL, 0.183 mmol), and morpholine (4.79 mg, 0.055 mmol) in ACN (1.0 mL) was added BOP (24.32 mg, 0.055 mmol), and the mixture was stirred at 60° C. for 18 hours. The mixture was concentrated to provide crude 4-(7-(8-ethyl-3-(methoxymethoxy)naphthalen-1-yl)-8-fluoro-2-(((4aS,7aR)-1-methyloctahydro-4aH-cyclopenta[b]pyridin-4a-yl)methoxy)quinazolin-4-yl)morpholine. MS(ESI) m/z: 615.3 [M+H]⁺. A mixture of 4-(7-(8-ethyl-3-(methoxymethoxy)naphthalen-1-yl)-8-fluoro-2-(((4aS,7aR)-1-methyloctahydro-4aH-cyclopenta[b]pyridin-4a-yl)methoxy)quinazolin-4-yl)morpholine, triethylsilane (1 drop), water (1 drop) and TFA (1.0 mL) was stirred at room temperature for 1 hour. The mixture was concentrated. The crude product was purified by prep-HPLC (Phenomenex, Luna 5 micron 30×100 mm, flow rate=40 ml/min., gradient=0% A to 100% B in 12 min., A=H₂O/ACN/TFA (90:10:0.1), B=H₂O/ACN/TFA (10:90:0.1)). The pure fraction was loaded onto an Oasis MCX cation mixed-mode polymer cartridge (150 mg), the cartridge was washed with methanol (30 mL) and the product was eluted with 0.1 N ammonia in methanol (5.0 mL). The ammonia eluant was concentrated. The pure product was lyophilized with ACN/H₂O (1:1, 5 mL) to provide 5-ethyl-4-(8-fluoro-2-(((4aS,7aR)-1-methyloctahydro-4aH-cyclopenta[b]pyridin-4a-yl)methoxy)-4-morpholinoquinazolin-7-yl)naphthalen-2-ol (11.75 mg, 0.019 mmol, 50.6% yield) as a white powder. MS(ESI) m/z: 571.4 [M+H]⁺. ¹H NMR (499 MHz, METHANOL-d₄) δ 7.83 (d, J=7.9 Hz, 1H), 7.62 (d, J=8.1 Hz, 1H), 7.37-7.23 (m, 3H), 7.14 (d, J=6.5 Hz, 1H), 6.93 (d, J=2.6 Hz, 1H), 4.55 (dd, J=10.8, 6.6 Hz, 1H), 4.29 (dd, J=10.8, 6.5 Hz, 1H), 3.98-3.90 (m, 8H), 2.95-2.86 (m, 1H), 2.73-2.63 (m, 1H), 2.48-2.36 (m, 3H), 2.34 (s, 3H), 1.96 (br dd, J=10.8, 5.3 Hz, 1H), 1.91-1.68 (m, 8H), 1.65-1.54 (m, 1H), 0.91-0.85 (m, 3H).

### Preparation of Intermediate 54: (1S,4S)-5-(2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoroquinazolin-4-yl)-2,5-diazabicyclo[2.2.1]heptan-3-one

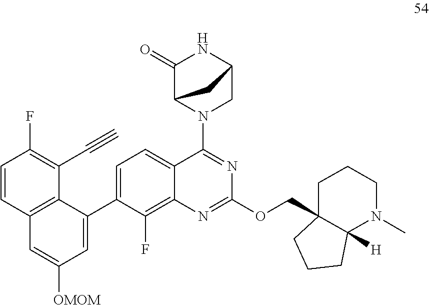

To a stirred solution of 2-{[(4aS,7aR)-1-methyl-octahydro-1H-cyclopenta[b]pyridin-4a-yl]methoxy}-7-[8-ethynyl-7-fluoro-3,3-bis(methoxymethoxy)naphthalen-1-yl]-8-fluoroquinazolin-4-ol (30 mg, 0.054 mmol) in ACN (1 mL) was added (1S,4S)-2,5-diazabicyclo[2.2.1]heptan-3-one (6.61 mg, 0.059 mmol), DIPEA (0.0286 mL, 0.164 mmol) and BOP (35.6 mg, 0.080 mmol). The reaction mixture was heated at 80° C. for 8h. The reaction mixture was then cooled to room temperature, the solvent was evaporated under reduced pressure and the residue was diluted with ethyl acetate. The organic layer was washed with water and brine, and then dried over anhydrous sodium sulfate, filtered, and concentrated under reduced pressure to provide the crude residue. The crude material was purified by COMBIFLASH™ chromatography (Teledyne ISO, Lincoln, NE) (40 g REDISEP™ column (Teledyne ISCO, Lincoln, NE), eluting with a gradient from 60-100% EtOAc in petroleum ether). Fractions containing the desired product were evaporated to provide (1S,4S)-5-(2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoroquinazolin-4-yl)-2,5diazabicyclo[2.2.1]heptan-3-one (30 mg, 0.046 mmol, 86% yield) as brown solid. MS(ESI) m/z: 654.3 [M+H]⁺.

### Example 5-1: (1S,4S)-5-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoroquinazolin-4-yl)-2,5-diazabicyclo[2.2.1]heptan-3-one

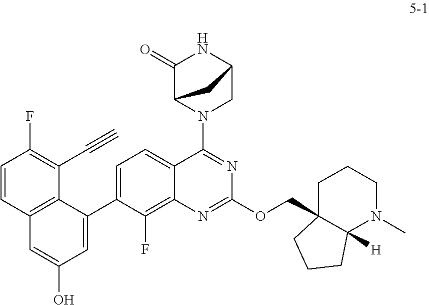 [SMILES: C#Cc1c(F)ccc2cc(O)cc(-c3ccc4c(N5C[C@@H]6C[C@H]5C(=O)N6)nc(OC[C@]56CCC[C@H]5N(C)CCC6)nc4c3F)c12]

To a stirred solution of (1S,4S)-5-(2-{[(4aS,7aR)-1-methyl-octahydro-1H-cyclopenta[b]pyridin-4a-yl]methoxy}-7-[8-ethynyl-7-fluoro-3-(methoxymethoxy)naphthalen-1-yl]-8-fluoroquinazolin-4-yl)-2,5-diazabicyclo[2.2.1]heptan-3-one (30 mg, 0.046 mmol) in acetonitrile (1 mL) at 0° C. was added 4N HCl (4M in dioxane) (0.115 mL, 0.459 mmol), and the reaction mixture was stirred at room temperature for 1 h. The volatiles from the reaction mixture were removed under reduced pressure (at lower temperature, ˜30° C.), and the crude residue was co-evaporated with 1,4-dioxane. Then the crude residue was dissolved in 1,4-dioxane (2 mL) and excess TEA (1 mL) was added, and the mixture was concentrated under reduced pressure to provide an off-white solid. The crude compound was purified by prep-HPLC [HPLC Method: Column: CHIRALPAK™ IC (Daicel, Japan) (250 mm×21 mm); Mobile Phase A: 0.1% DEA in CAN Mobile Phase B: 0.1% DEA in Methanol Flow rate: 15.0 ml/min Gradient: T/% B:0/50; 10/50] to provide (1S,4S)-5-(2-{[(4aS,7aR)-1-methyl-octahydro-1H-cyclopenta[b]pyridin-4a-yl]methoxy}-7-(8-ethynyl-7-fluoro-3-hydroxynaphthalen-1-yl)-8-fluoroquinazolin-4-yl)-2,5-diazabicyclo[2.2.1]heptan-3-one (4.1 mg, 6.72 mol, 14.65% yield) as an off white solid MS(ESI) m/z: 610.3 [M+H]⁺. ¹H NMR (400 MHz, DMSO-d₆) δ ppm=10.16 (s, 1H), 7.97 (dd, J=6.0, 9.0 Hz, 1H), 7.75 (d, J=8.5 Hz, 1H), 7.46 (t, J=8.9 Hz, 1H), 7.37 (d, J=2.8 Hz, 1H), 7.21 (t, J=7.4 Hz, 1H), 7.07 (d, J=2.3 Hz, 1H), 4.49-4.40 (m, 1H), 4.38-4.21 (m, 2H), 4.11 (dd, J=2.3, 10.8 Hz, 1H), 3.85 (d, J=4.0 Hz, 1H), 3.83-3.78 (m, 2H), 3.56-3.51 (m, 4H), 2.82-2.71 (m, 1H), 2.60-2.54 (m, 1H), 2.32-2.17 (m, 4H), 1.90-1.47 (m, 8H), 1.45-1.34 (m, 1H).

### Preparation of Intermediate 56: tert-butyl 3-(2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoropyrido[4,3-d]pyrimidin-4-yl)-3,8-diazabicyclo[3.2.1]octane-8-carboxylate

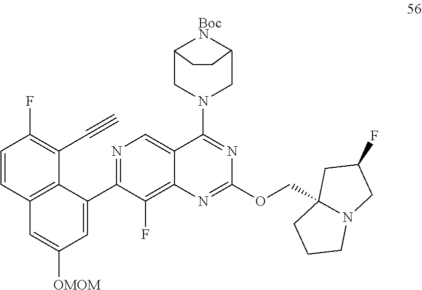

To a stirred solution of tert-butyl 3-(2-{[(2R,7aS)-2-fluoro-hexahydro-1H-pyrrolizin-7a-yl]methoxy}-8-fluoro-7-[7-fluoro-3-(methoxymethoxy)-8-{2-[tris(propan-2-yl)silyl]ethynyl}naphthalen-1-yl]pyrido[4,3-d]pyrimidin-4-yl)-3,8-diazabicyclo[3.2.1]octane-8-carboxylate (3.7 g, 4.11 mmol) in DMF (50 mL) was added CsF (6.24 g, 41.1 mmol). The reaction mixture was heated at 50° C. for 1 h. The reaction mixture was then cooled to room temperature, diluted with water, and extracted with EtOAc. The combined organic layers were washed with brine, dried over anhydrous sodium sulfate, filtered, and concentrated under reduced pressure to provide tert-butyl 3-(2-{[(2R,7aS)-2-fluoro-hexahydro-1H-pyrrolizin-7a-yl]methoxy}-7-[8-ethynyl-7-fluoro-3-(methoxymethoxy)naphthalen-1-yl]-8-fluoropyrido[4,3-d]pyrimidin-4-yl)-3,8-diazabicyclo[3.2.1]octane-8-carboxylate (2.9 g, 3.89 mmol, 95% yield) as a brown liquid, which was taken for the next step without further purification. MS(ESI) m/z: 746.6 [M+H]⁺.

### Preparation of Intermediate 57: 4-(2-{[(2R,7aS)-2-fluoro-hexahydro-1H-pyrrolizin-7a-yl]methoxy}-4-{3,8-diazabicyclo[3.2.1]octan-3-yl}-8-fluoropyrido[4,3-d]pyrimidin-7-yl)-5-ethynyl-6-fluoronaphthalen-2-ol

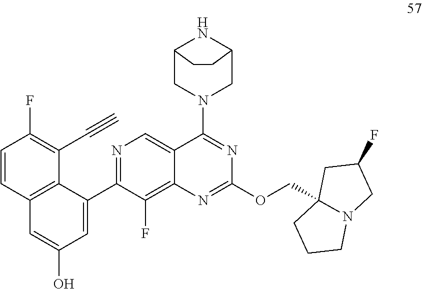 [SMILES: C#Cc1c(F)ccc2cc(O)cc(-c3ncc4c(N5CCNCC5)nc(OC[C@@]56CCCN5C[C@H](F)C6)nc4c3F)c12]

To a solution tert-butyl 3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-3,8-diazabicyclo[3.2.1]octane-8-carboxylate (700 mg, 0.940 mmol) in acetonitrile (5 mL), was added 4M HCl in dioxane (0.29 mL, 9.40 mmol) at 0° C. and the mixture was stirred for 1 h. Then the solvent was removed under reduced pressure, the residue was diluted with DCM and neutralized with excess TEA. The organic layer was washed with water and brine solution, then dried over Na₂SO₄, filtered, and concentrated under reduced pressure to provide 4-(2-{[(2R,7aS)-2-fluoro-hexahydro-1H-pyrrolizin-7a-yl]methoxy}-4-{3,8-diazabicyclo[3.2.1]octan-3-yl}-8-fluoropyrido[4,3-d]pyrimidin-7-yl)-5-ethynyl-6-fluoronaphthalen-2-ol (300 mg, 0.50 mmol, 53% yield). MS(ESI) m/z: 601.3 [M+H]⁺.

### Example 6-1: 1-[3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-3,8-diazabicyclo[3.2.1]octan-8-yl]-2,2,2-trifluoroethan-1-one

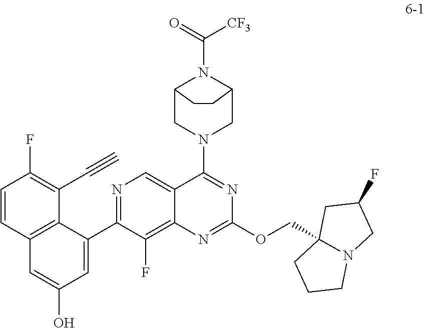 [SMILES: C#Cc1c(F)ccc2cc(O)cc(-c3ncc4c(N5CCN(C(=O)C(F)(F)F)CC5)nc(OC[C@@]56CCCN5C[C@H](F)C6)nc4c3F)c12]

To a solution of 4-(2-{[(2R,7aS)-2-fluoro-hexahydro-1H-pyrrolizin-7a-yl]methoxy}-4-{3,8-diazabicyclo[3.2.1]octan-3-yl}-8-fluoropyrido[4,3-d]pyrimidin-7-yl)-5-ethynyl-6-fluoronaphthalen-2-ol (50 mg, 0.083 mmol) in DCM (2 mL) was added TEA (0.035 mL, 0.250 mmol) and TFAA (0.018 mL, 0.125 mmol) at 0° C. and the mixture was stirred for 1 h. The reaction mixture was then quenched with water and extracted with DCM. The combined organic layers were washed with brine, dried over anhydrous sodium sulfate, filtered, and evaporated under reduced pressure to provide the crude product. The crude compound was purified by prep-HPLC. [HPLC Method: Preparative column: YMC ExRS (20×250) 5u, Mobile phase A: 10 mM Ammonium bicarbonate in water-9.5 pH; Mobile phase B: acetonitrile:MeOH (1:1); Gradient=0 (min)−60%, 2-65%, 15-85%, 16-70%, 17-100%; Temperature: 27° C.; Flow rate: 19 mL/min; Detection: UV at 220 &254 nm] to provide 1-[3-(2-{[(2R,7aS)-2-fluoro-hexahydro-1H-pyrrolizin-7a-yl]methoxy}-7-(8-ethynyl-7-fluoro-3-hydroxynaphthalen-1-yl)-8-fluoropyrido[4,3-d]pyrimidin-4-yl)-3,8-diazabicyclo[3.2.1]octan-8-yl]-2,2,2-trifluoroethan-1-one (15 mg, 0.019 mmol, 23% yield). MS(ESI) m/z: 697.2, [M+H]⁺. ¹H NMR (400 MHz, DMSO-d₆) δ ppm=9.06 (s, 1H), 7.94 (dd, J=9.1, 5.9 Hz, 1H), 7.44 (t, J=9.0 Hz, 1H), 7.35 (d, J=2.3 Hz, 1H), 7.17 (s, 1H), 5.40-5.18 (m, 1H), 4.81 (br s, 1H), 4.76-4.62 (m, 2H), 4.59-4.52 (m, 1H), 4.18-4.11 (m, 1H), 4.08-4.02 (m, 1H), 3.91 (d, J=6.0 Hz, 1H), 3.79-3.68 (m, 2H), 3.13-3.01 (m, 4H), 2.84 (br d, J=6.5 Hz, 1H), 2.17-1.74 (m, 9H).

The examples in Table 3 were prepared according to procedures described for Example 6-1 from appropriate starting materials.

TABLE 3

| | | | LCMS | |
|---|---|---|---|---|
| # | Structure | IUPAC | (M + H)⁺ | ¹H NMR |
| 6-2 | 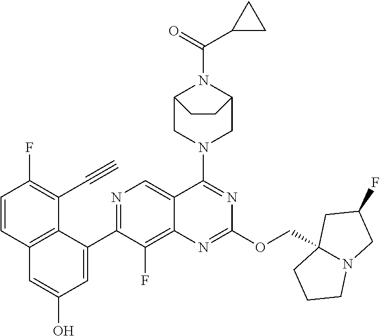 [SMILES: C#Cc1c(F)ccc2cc(O)cc(-c3ncc4c(N5CC6CCC(C5)N6C(=O)C5CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-{8- cyclopropanecar- bonyl-3,8- diazabicyclo[3.2.1]oc- tan-3-yl}-8- fluoropyrido[4,3- d]pyrimidin-7-yl)-5- ethynyl-6- fluoronaphthalen-2- ol | 669.1 | ¹H NMR (400 MHz, DMSO-d₆) δ ppm = 10.16 (s, 1H), 9.08 (s, 1H), 7.98 (dd, J = 5.9, 9.1 Hz, 1H), 7.47 (t, J = 9.0 Hz, 1H), 7.40 (d, J = 2.8 Hz, 1H), 7.19 (d, J = 1.8 Hz, 1H), 5.26 (br s, 1H), 4.82 (br dd, J = 1.9, 3.6 Hz, 1H), 4.72- 4.55 (m, 2H), 4.50-4.40 (m, 1H), 4.18 (s, 1H), 3.94 (s, 1H), 3.80- 3.52 (m, 1H), 3.10 (br s, 2H), 2.21-1.67 (m, 10H), 0.83 (br d, J = 4.8 Hz, 4H). |
| 6-3 | 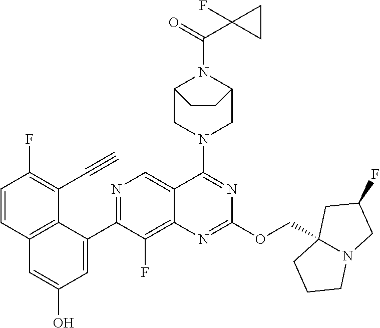 [SMILES: C#Cc1c(F)ccc2cc(O)cc(-c3ncc4c(N5CC6CCC(C5)N6C(=O)C5(F)CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[8-(1- fluorocyclopropanecar- bonyl)-3,8- diazabicyclo[3.2.1]oc- tan-3-yl]pyrido[4,3- d]pyrimidin-7-yl)-5- ethynyl-6- fluoronaphthalen-2- ol | 687.3 | ¹H NMR (400 MHz, DMSO-d₆) δ ppm = 10.18 (s, 1H), 9.11 (br d, J = 1.3 Hz, 1H), 7.99 (dd, J = 5.8, 9.3 Hz, 1H), 7.47 (t, J = 9.1 Hz, 1H), 7.41 (d, J = 2.5 Hz, 1H), 7.18 (d, J = 2.3 Hz, 1H), 4.80 (br s, 1H), 4.73-4.41 (m, 1H), 3.92 (s, 1H), 3.74 (s, 1H), 1.85 (br s, 4H), 1.39-1.25 (m, 4H). |
| 6-4 | 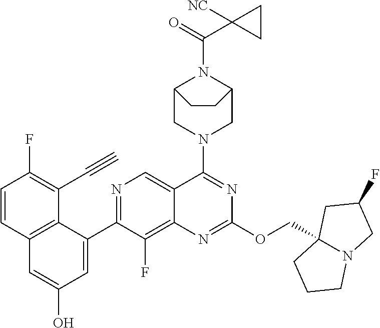 [SMILES: C#Cc1c(F)ccc2cc(O)cc(-c3ncc4c(N5CC6CCC(C5)N6C(=O)C5(C#N)CC5)nc(OC[C@@]56CCCN5C[C@H](F)C6)nc4c3F)c12] | 1-[3-(2-{[(2R,7aS)- 2-fluoro-hexahydro- 1H-pyrrolizin-7a- yl]methoxy}-7-(8- ethynyl-7-fluoro-3- hydroxynaphthalen- 1-yl)-8- fluoropyrido[4,3- d]pyrimidin-4-yl)- 3,8- diazabicyclo[3.2.1]oc- tane-8- carbonyl]cyclopropane- 1-carbonitrile | 694.3 | |
| 6-5 | 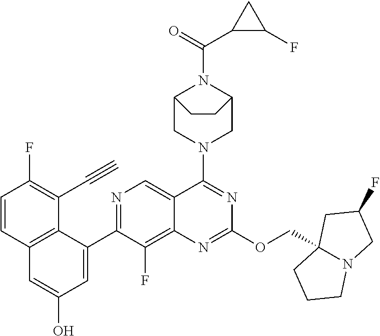 [SMILES: C#Cc1c(F)ccc2cc(O)cc(-c3ncc4c(N5CC6CCC(C5)N6C(=O)C5CC5F)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[8-(2- fluorocyclopropanecar- bonyl)-3,8- diazabicyclo[3.2.1]oc- tan-3-yl]pyrido[4,3- d]pyrimidin-7-yl)-5- ethynyl-6- fluoronaphthalen-2- ol | 687.3 | |
| 6-6 | 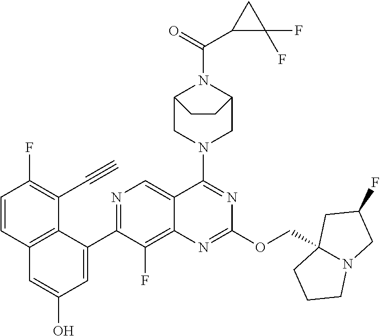 [SMILES: C#Cc1c(F)ccc2cc(O)cc(-c3ncc4c(N5CC6CCC(C5)N6C(=O)C5CC5(F)F)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-[8- (2,2- difluorocyclopropane- carbonyl)-3,8- diazabicyclo[3.2.1]oc- tan-3-yl]-8- fluoropyrido[4,3- d]pyrimidin-7-yl)-5- ethynyl-6- fluoronaphthalen-2- ol, isomer 1 | 705.2 | |
| 6-7 | 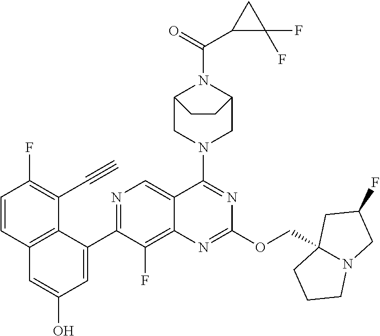 [SMILES: C#Cc1c(F)ccc2cc(O)cc(-c3ncc4c(N5CC6CCC(C5)N6C(=O)C5CC5(F)F)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-[8- (2,2- difluorocyclopropane- carbonyl)-3,8- diazabicyclo[3.2.1]oc- tan-3-yl]-8- fluoropyrido[4,3- d]pyrimidin-7-yl)-5- ethynyl-6- fluoronaphthalen-2- ol, isomer 2 | 705.3 | |
| 6-8 | 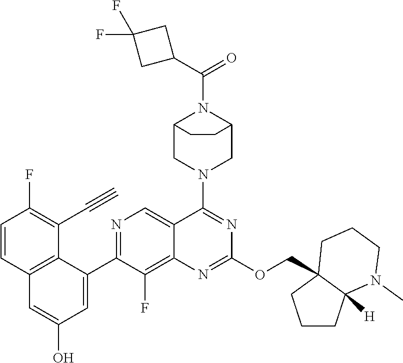 [SMILES: C#Cc1c(F)ccc2cc(O)cc(-c3ncc4c(N5CC6CCC(C5)N6C(=O)C5CC(F)(F)C5)nc(OC[C@]56CCC[C@H]5N(C)CCC6)nc4c3F)c12] | 4-(2-{[(4aS,7aR)-1- methyl-octahydro- 1H- cyclopenta[b]pyridin- 4a-yl]methoxy}-4- [8-(3,3- difluorocyclobutane- carbonyl)-3,8- diazabicyclo[3.2.1]oc- tan-3-yl]-8- fluoropyrido[4,3- d]pyrimidin-7-yl)-5- ethynyl-6- fluoronaphthalen-2- ol | 729.3 | |
| 6-9 | 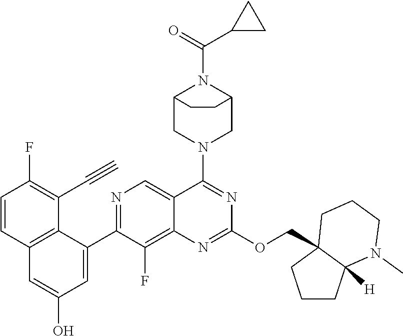 [SMILES: C#Cc1c(F)ccc2cc(O)cc(-c3ncc4c(N5CC6CCC(C5)N6C(=O)C5CC5)nc(OC[C@]56CCC[C@H]5N(C)CCC6)nc4c3F)c12] | 4-(2-{[(4aS,7aR)-1- methyl-octahydro- 1H- cyclopenta[b]pyridin- 4a-yl]methoxy}-4- {8- cyclopropanecarbonyl- 3,8- diazabicyclo[3.2.1]oc- tan-3-yl}-8- fluoropyrido[4,3- d]pyrimidin-7-yl)-5- ethynyl-6- fluoronaphthalen-2- ol | 679.3 | |
| 6-10 | 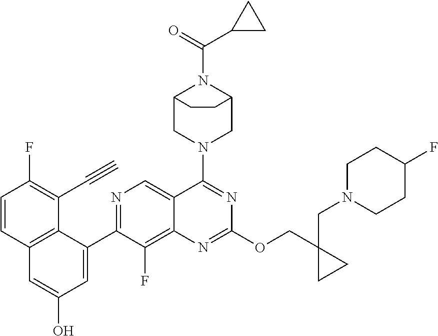 [SMILES: C#Cc1c(F)ccc2cc(O)cc(-c3ncc4c(N5CC6CCC(C5)N6C(=O)C5CC5)nc(OCC5(CN6CCC(F)CC6)CC5)nc4c3F)c12] | 4-(4-{8- cyclopropanecarbonyl- 3,8- diazabicyclo[3.2.1]oc- tan-3-yl}-8-fluoro- 2-({1-[(4- fluoropiperidin-1- yl)methyl]cyclopropyl} methoxy)pyrido[4,3- d]pyrimidin-7- yl)-5-ethynyl-6- fluoronaphthalen-2- ol | 697.3 | |
| 6-11 | 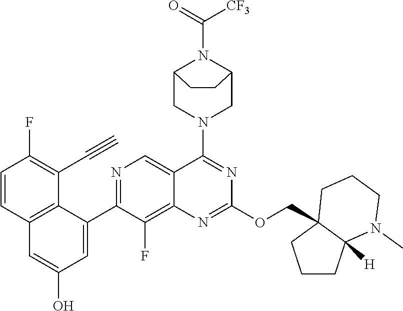 [SMILES: C#Cc1c(F)ccc2cc(O)cc(-c3ncc4c(N5CC6CCC(C5)N6C(=O)C(F)(F)F)nc(OC[C@]56CCC[C@H]5N(C)CCC6)nc4c3F)c12] | 1-[3-(2-{[(4aS,7aR)- 1-methyl-octahydro- 1H- cyclopenta[b]pyridin- 4a-yl]methoxy}-7- (8-ethynyl-7-fluoro- 3- hydroxynaphthalen- 1-yl)-8- fluoropyrido[4,3- d]pyrimidin-4-yl)- 3,8- diazabicyclo[3.2.1]oc- tan-8-yl]-2,2,2- trifluoroethan-1-one | 707.2 | ¹H NMR (400 MHz, DMSO-d₆) δ ppm = 10.17 (s, 1H), 9.06 (s, 1H), 7.99 (dd, J = 9.1 Hz, 6.0 Hz, 1H), 7.47 (t, J = 9.0 Hz, 1H), 7.41 (d, J = 2.3 Hz, 1H), 7.19 (br s, 1H), 4.82 (br s, 1H), 4.77-4.63 (m, 2H), 4.59-4.44 (m, 2H), 4.17 (br d, J = 10.8 Hz, 1H), 3.98-3.92 (m, 1H), 3.79-3.66 (m, 2H), 2.68 (br s, 2H), 2.34 (br s, 1H), 2.27-2.15 (m, 4H), 2.07- 1.79 (m, 5H), 1.76-1.48 (m, 7H), 1.41 (br d, J = 9.6 Hz, 1H) |

### Preparation of Intermediate 58: 2-{[(2R,7aS)-2-fluoro-hexahydro-1H-pyrrolizin-7a-yl]methoxy}-7-bromo-4-[8-(4,4-difluorocyclohexanecarbonyl)-3,8-diazabicyclo[3.2.1]octan-3-yl]-8-fluoroquinazoline

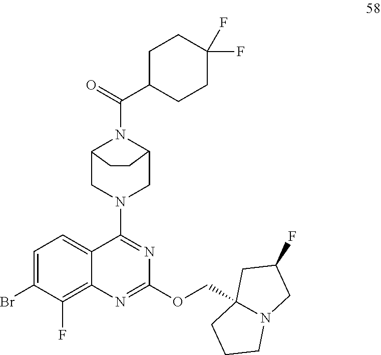 [SMILES: O=C(C1CCC(F)(F)CC1)N1CCN(c2nc(OC[C@@]34CCCN3C[C@H](F)C4)nc3c(F)c(Br)ccc23)CC1]

To a stirred solution of 2-{[(2R,7aS)-2-fluoro-hexahydro-1H-pyrrolizin-7a-yl]methoxy}-7-bromo-4-{3,8-diazabicyclo[3.2.1]octan-3-yl}-8-fluoroquinazoline (150 mg, 0.303 mmol) in DCM (10 mL) was added DIPEA (0.16 mL, 0.910 mmol) and propylphosphonic anhydride solution in EtOAc (0.4 mL, 0.61 mmol) at 0° C. After being stirred for 15 min at 0° C., 4,4-difluorocyclohexane-1-carboxylic acid (54.8 mg, 0.33 mmol) was added and the reaction mixture was allowed to warm to room temperature and stirred for 16 h. The reaction mixture was then extracted with EtOAc (2×), washed with water and brine solution, and dried over sodium sulfate, filtered, and concentrated to provide 2-{[(2R,7aS)-2-fluoro-hexahydro-1H-pyrrolizin-7a-yl]methoxy}-7-bromo-4-[8-(4,4-difluorocyclohexanecarbonyl)-3,8-diazabicyclo[3.2.1]octan-3-yl]-8-fluoroquinazoline (120 mg, 0.18 mmol, 53% yield). MS(ESI) m/z: 642.2 (M+2)+.

### Preparation of Intermediate 59: 2-{[(2R,7aS)-2-fluoro-hexahydro-1H-pyrrolizin-7a-yl]methoxy}-4-[8-(4,4-difluorocyclohexanecarbonyl)-3,8-diazabicyclo[3.2.1]octan-3-yl]-8-fluoro-7-[7-fluoro-3-(methoxymethoxy)-8-{2-[tris(propan-2-yl)silyl]ethynyl}naphthalen-1-yl]quinazoline

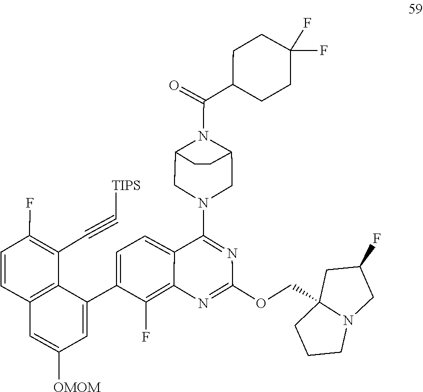

To a stirred solution of 2-{[(2R,7aS)-2-fluoro-hexahydro-1H-pyrrolizin-7a-yl]methoxy}-7-bromo-4-[8-(4,4-difluorocyclohexanecarbonyl)-3,8-diazabicyclo[3.2.1]octan-3-yl]-8-fluoroquinazoline (100 mg, 0.156 mmol) in 1,4-dioxane (5 mL), were added ((2-fluoro-6-(methoxymethoxy)-8-(4,4,5,5-tetramethyl-1,3,2-dioxaborolan-2-yl)naphthalen-1-yl)ethynyl)triisopropylsilane (120 mg, 0.234 mmol) and aqueous 2M potassium phosphate, tribasic (0.23 mL, 0.468 mmol). The reaction mixture was degassed under argon for 5 minutes, and then [1,1′-bis(di-tertbutylphosphino)ferrocene]dichloropalladium(II) (10.2 mg, 0.016 mmol) was added and the mixture was again degassed under argon for 3 minutes and then heated at 50° C. for 16 h. The reaction mixture was cooled to room temperature, diluted with water, and extracted with EtOAc. The combined organic layers were washed with brine, dried over anhydrous sodium sulfate, filtered, and concentrated under reduced pressure to provide a crude residue which was purified by COMBFLASH™ chromatography (Teledyne ISO, Lincoln, NE) (12 g silica gel column, using 2-5% MeOH in DCM) to provide 2-{[(2R,7aS)-2-fluoro-hexahydro-1H-pyrrolizin-7a-yl]methoxy}-4-[8-(4,4-difluorocyclohexanecarbonyl)-3,8-diazabicyclo[3.2.1]octan-3-yl]-8-fluoro-7-[7-fluoro-3-(methoxymethoxy)-8-{2-[tris(propan-2-yl)silyl]ethynyl}naphthalen-1-yl]quinazoline (126 mg, 0.133 mmol, 85% yield) as a brown solid. MS(ESI) m/z: 946.4 [M+H]⁺.

### Preparation of Intermediate 60: 2-{[(2R,7aS)-2-fluoro-hexahydro-1H-pyrrolizin-7a-yl]methoxy}-4-[8-(4,4-difluorocyclohexanecarbonyl)-3,8-diazabicyclo[3.2.1]octan-3-yl]-7-[8-ethynyl-7-fluoro-3-(methoxymethoxy)naphthalen-1-yl]-8-fluoroquinazoline

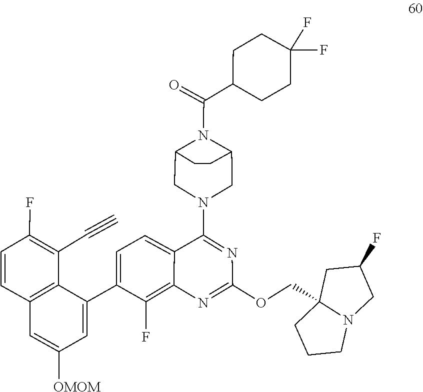

To a stirred solution of 2-{[(2R,7aS)-2-fluoro-hexahydro-1H-pyrrolizin-7a-yl]methoxy}-4-[8-(4,4-difluorocyclohexanecarbonyl)-3,8-diazabicyclo[3.2.1]octan-3-yl]-8-fluoro-7-[7-fluoro-3-(methoxymethoxy)-8-{2-[tris(propan-2-yl)silyl]ethynyl}naphthalen-1-yl]quinazoline (126 mg, 0.133 mmol) in DMF (5 mL) was added CsF (202 mg, 1.33 mmol). The reaction mixture was heated at 50° C. for 1 h. The reaction mixture was then diluted with water and extracted with EtOAc. The combined organic layers were washed with brine, dried over anhydrous sodium sulfate, filtered, and concentrated under reduced pressure to provide 2-{[(2R,7aS)-2-fluoro-hexahydro-1H-pyrrolizin-7a-yl]methoxy}-4-[8-(4,4-difluorocyclohexanecarbonyl)-3,8-diazabicyclo[3.2.1]octan-3-yl]-7-[8-ethynyl-7-fluoro-3-(methoxymethoxy)naphthalen-1-yl]-8-fluoroquinazoline (99 mg, 0.125 mmol, 94% yield) as a pale brown liquid, which was taken for next step without purification. MS(ESI) m/z: 790.3 [M+H]⁺.

### Example 7-1: 4-(2-{[(2R,7aS)-2-fluoro-hexahydro-1H-pyrrolizin-7a-yl]methoxy}-4-[8-(4,4-difluorocyclohexanecarbonyl)-3,8-diazabicyclo[3.2.1]octan-3-yl]-8-fluoroquinazolin-7-yl)-5-ethynyl-6-fluoronaphthalen-2-ol

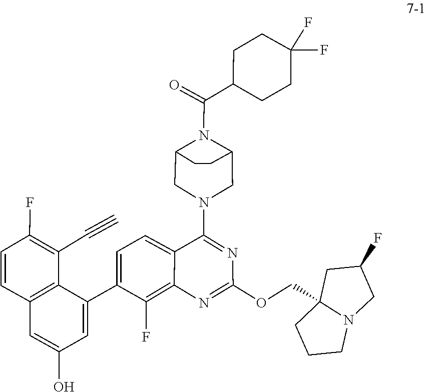 [SMILES: C#Cc1c(F)ccc2cc(O)cc(-c3ccc4c(N5CC6CCC(C5)N6C(=O)C5CCC(F)(F)CC5)nc(OC[C@@]56CCCN5C[C@H](F)C6)nc4c3F)c12]

To a stirred solution 2-{[(2R,7aS)-2-fluoro-hexahydro-1H-pyrrolizin-7a-yl]methoxy}-4-[8-(4,4-difluorocyclohexanecarbonyl)-3,8-diazabicyclo[3.2.1]octan-3-yl]-7-[8-ethynyl-7-fluoro-3-(methoxymethoxy)naphthalen-1-yl]-8-fluoroquinazoline (99 mg, 0.125 mmol) in acetonitrile (5 mL), was added 4M HCl in dioxane (5 mL, 2.51 mmol) at 0° C. and the mixture was stirred for 2 h. The volatiles from the reaction mixture were removed under reduced pressure and the crude residue was co-evaporated with 1,4-dioxane. Then, the crude residue was dissolved in 1,4-dioxane (2 mL) and excess TEA (1 mL) was added. The mixture was concentrated under reduced pressure to give an off-white solid. The crude compound was purified by prep-HPLC [Waters XBridge C18 (Waters Corp., Milford, CT), (250 mm×19 mm, 5 μm particles); Mobile Phase A: 5:95 acetonitrile: water with 10-mM ammonium acetate; Mobile Phase B: 95:5 acetonitrile: water with 10 mM ammonium acetate; Gradient: a 0-minute hold at 15% B, 15-55% B over 25 minutes, then a 5-minute hold at 100% B; Flow Rate: 20 mL/min; Detection: UV at 220 & 254 nm] to provide 4-(2-{[(2R,7aS)-2-fluoro-hexahydro-1H-pyrrolizin-7a-yl]methoxy}-4-[8-(4,4-difluorocyclohexanecarbonyl)-3,8-diazabicyclo[3.2.1]octan-3-yl]-8-fluoroquinazolin-7-yl)-5-ethynyl-6-fluoronaphthalen-2-ol (8.7 mg, 0.012 mmol, 9% yield) as a off-white solid. MS(ESI) m/z: 746.3 [M+H]⁺.

The examples in Table 4 were prepared according to procedures described for Examples 6-1 and 7-1 from appropriate starting materials.

TABLE 4

| | | | LCMS | |
|---|---|---|---|---|
| # | Structure | IUPAC | (M + H)⁺ | ¹H NMR |
| 7-2 | 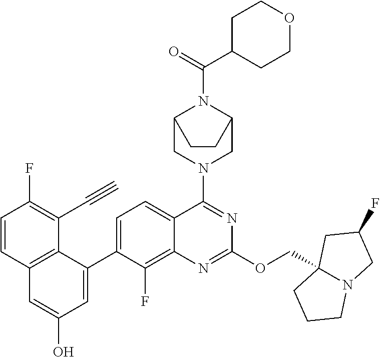 [SMILES: C#Cc1c(F)ccc2cc(O)cc(-c3ccc4c(N5CC6CCC(C5)N6C(=O)C5CCOCC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-[8-(oxane- 4-carbonyl)-3,8- diazabicyclo[3.2.1] octan-3- yl]quinazolin-7-yl)- 5-ethynyl-6- fluoronaphthalen- 2-ol | 712.3 | |
| 7-3 | 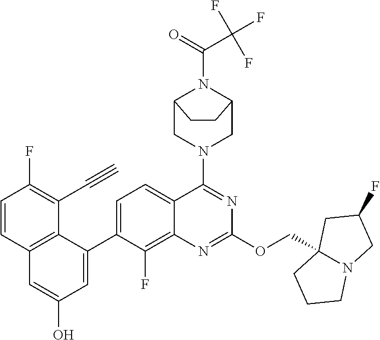 [SMILES: C#Cc1c(F)ccc2cc(O)cc(-c3ccc4c(N5CC6CCC(C5)N6C(=O)C(F)(F)F)nc(OC[C@@]56CCCN5C[C@H](F)C6)nc4c3F)c12] | 1-[3-(2-{[(2R,7aS)- 2-fluoro- hexahydro-1H- pyrrolizin-7a- yl]methoxy}-7-(8- ethynyl-7-fluoro-3- hydroxynaphthalen- 1-yl)-8- fluoroquinazolin-4- yl)-3,8- diazabicyclo[3.2.1] octan-8-yl]-2,2,2- trifluoroethan-1- one | 696.3 | ¹H NMR (400 MHz, DMSO-d₆) δ ppm 10.12 (s, 1H), 7.97 (dd, J = 6.0, 9.0 Hz, 1H), 7.79-7.73 (m, 1H), 7.51-7.43 (m, 1H), 7.39-7.34 (m, 1H), 7.27-7.21 (m, 1H), 7.11- 7.05 (m, 1H), 5.37-5.19 (m, 1H), 4.83-4.77 (m, 1H), 4.70-4.59 (m, 1H), 4.58-4.39 (m, 2H), 4.14- 3.98 (m, 2H), 3.85 (d, J = 1.5 Hz, 1H), 3.63-3.54 (m, 2H), 3.13-3.01 (m, 3H), 2.87-2.80 (m, 1H), 2.17-2.11 (m, 1H), 2.08- 1.90 (m, 6H), 1.89-1.76 (m, 3H) |
| 7-4 | 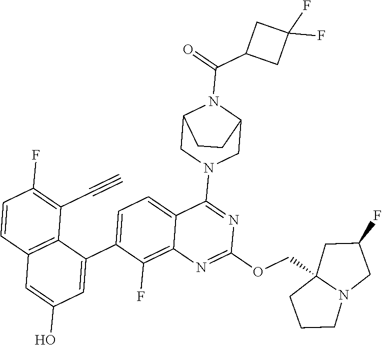 [SMILES: C#Cc1c(F)ccc2cc(O)cc(-c3ccc4c(N5CC6CCC(C5)N6C(=O)C5CC(F)(F)C5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-[8- (3,3- difluorocyclobutane- carbonyl)-3,8- diazabicyclo[3.2.1] octan-3-yl]-8- fluoroquinazolin-7- yl)-5-ethynyl-6- fluoronaphthalen- 2-ol | 718.3 | |
| 7-5 | 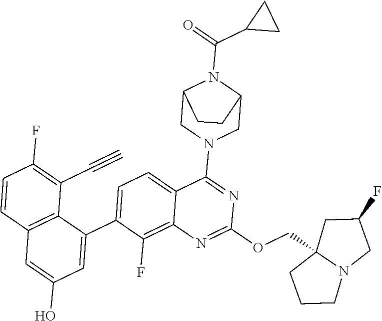 [SMILES: C#Cc1c(F)ccc2cc(O)cc(-c3ccc4c(N5CC6CCC(C5)N6C(=O)C5CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-4-{8- cyclopropanecarbo- nyl-3,8- diazabicyclo[3.2.1] octan-3-yl}-8- fluoroquinazolin-7- yl)-5-ethynyl-6-7- 6fluoronaphthalen- 2-ol | 668.1 | |
| 7-6 | 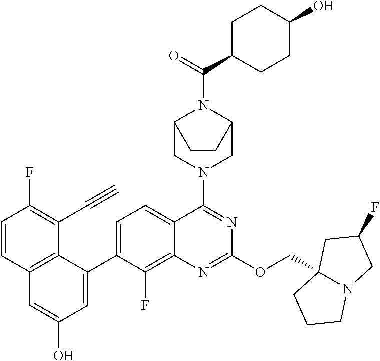 [SMILES: C#Cc1c(F)ccc2cc(O)cc(-c3ccc4c(N5CC6CCC(C5)N6C(=O)[C@H]5CC[C@@H](O)CC5)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{8- [(1s,4s)-4- hydroxycyclohexane- carbonyl]-3,8- diazabicyclo[3.2.1] octan-3- yl}quinazolin-7- yl)-5-ethynyl-6- fluoronaphthalen- 2-ol | 726.7 | |
| 7-7 | 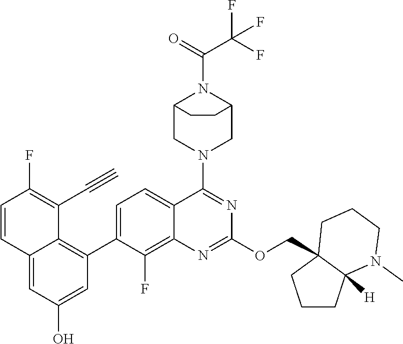 [SMILES: C#Cc1c(F)ccc2cc(O)cc(-c3ccc4c(N5CC6CCC(C5)N6C(=O)C(F)(F)F)nc(OC[C@]56CCC[C@H]5N(C)CCC6)nc4c3F)c12] | 1-[3-(2- {[(4aS,7aR)-1- methyl-octahydro- 1H- cyclopenta[b]pyridin- 4a-yl]methoxy}- 7-(8-ethynyl-7- fluoro-3- hydroxynaphthalen- 1-yl)-8- fluoroquinazolin-4- yl)-3,8- diazabicyclo[3.2.1] octan-8-yl]-2,2,2- trifluoroethan-1- one | 706.3 | |
| 7-8 | 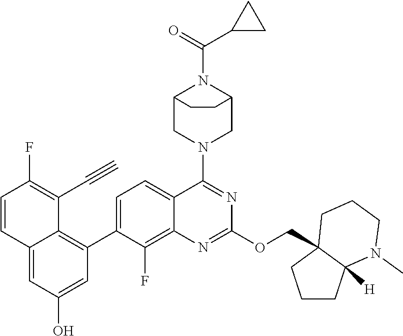 [SMILES: C#Cc1c(F)ccc2cc(O)cc(-c3ccc4c(N5CC6CCC(C5)N6C(=O)C5CC5)nc(OC[C@]56CCC[C@H]5N(C)CCC6)nc4c3F)c12] | 4-(2-{[(4aS,7aR)-1- methyl-octahydro- 1H- cyclopenta[b]pyridin- 4a-yl]methoxy}- 4-{8- cyclopropanecarbo- nyl-3,8- diazabicyclo[3.2.1] octan-3-yl}-8- fluoroquinazolin-7- yl)-5-ethynyl-6- fluoronaphthalen- 2-ol | 678.3 | |
| 7-9 | 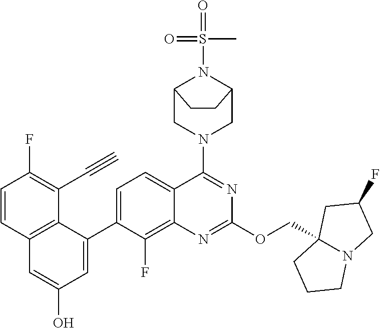 [SMILES: C#Cc1c(F)ccc2cc(O)cc(-c3ccc4c(N5CC6CCC(C5)N6S(C)(=O)=O)nc(OC[C@@]56CCCN5C[C@H](F)C6)nc4c3F)c12] | 4-(2-{[(2R,7aS)-2- fluoro-hexahydro- 1H-pyrrolizin-7a- yl]methoxy}-8- fluoro-4-{8- methanesulfonyl- 3,8- diazabicyclo[3.2.1] octan-3- yl}quinazolin-7- yl)-5-ethynyl-6- fluoronaphthalen- 2-ol | 678.0 | |

### Preparation of Intermediate 61: tert-butyl 3-(7-bromo-2,6-dichloro-8-fluoroquinazolin-4-yl)-3,8-diazabicyclo[3.2.1]octane-8-carboxylate

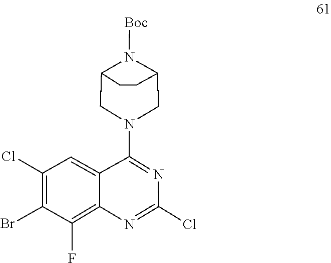 [SMILES: CC(C)(C)OC(=O)N1C2CCC1CN(c1nc(Cl)nc3c(F)c(Br)c(Cl)cc13)C2]

To a solution of 7-bromo-2,4,6-trichloro-8-fluoroquinazoline (300 mg, 3.03 mmol) in dioxane (8 mL) was added DIPEA (0.476 mL, 2.72 mmol) and tert-butyl 3,8-diazabicyclo[3.2.1]octane-8-carboxylate (193 mg, 0.908 mmol). The resulting mixture was stirred at 25° C. for 2 hours. The reaction mixture was concentrated under reduced pressure to give the crude product, which was diluted with EtOAc (50 mL) and washed with water (30 mL×2) and brine (50 mL). The organic phase was dried over anhydrous sodium sulfate, filtered, and concentrated under reduced pressure. The residue was purified by COMBFLASH™ chromatography (Teledyne ISO, Lincoln, NE) (12 g, ISCO column, MeOH/DCM, 0-5%, 20 min.) to provide tert-butyl 3-(7-bromo-2,6-dichloro-8-fluoroquinazolin-4-yl)-3,8-diazabicyclo[3.2.1]octane-8-carboxylate (415 mg, 0.82 mmol, 90% yield) as a white solid. MS (ESI) m/z 507.0 [M+2]⁺. ¹H NMR (499 MHz, DMSO-d₆) δ ppm=8.10 (d, J=1.9 Hz, 1H), 4.38 (br d, J=10.6 Hz, 2H), 4.25 (br s, 2H), 3.66 (m, 2H) 1.79 (m, 2H), 1.62 (m, 2H), 1.47 s, 9H).

### Preparation of Intermediate 62: tert-butyl 3-(7-bromo-6-chloro-2,8-difluoroquinazolin-4-yl)-3,8-diazabicyclo[3.2.1]octane-8-carboxylate

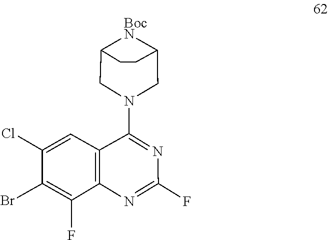 [SMILES: CC(C)(C)OC(=O)N1C2CCC1CN(c1nc(F)nc3c(F)c(Br)c(Cl)cc13)C2]

To a degassed solution of tert-butyl 3-(7-bromo-2,6-dichloro-8-fluoroquinazolin-4-yl)-3,8-diazabicyclo[3.2.1]octane-8-carboxylate (Intermediate 35, 1 g, 3.03 mmol) in DMA (80 mL) was added cesium fluoride (5.25 g, 34.6 mmol). The reaction mixture was degassed with nitrogen for 10 minutes and was heated at 88° C. for 5 h in a sealed tube. Water (200 mL) and EtOAc (150 mL) were added, and the reaction mixture was stirred for 15 minutes. The separated aqueous layer was extracted with EtOAc (2×100 mL) and the combined organic layers were dried over anhydrous Na₂SO₄, filtered, and concentrated under reduced pressure. The resulting residue was purified by COMBIFLASH™ chromatography (Teledyne ISO, Lincoln, NE) with 15-25% EtOAc in petroleum ether as an eluent to give tert-butyl-3-(7-bromo-6-chloro-2,8-difluoroquinazolin-4-yl)-3,8-diazabicyclo[3.2.1]octane-8-carboxylate (4.7 g, 8.77 mmol, 63.4% yield) as a pale-yellow solid. MS (ESI) m z 489.0 [M+1]⁺.

### Preparation of Intermediate 63: tert-butyl 3-(7-(6-(bis(4-methoxybenzyl)amino)-4-methylpyridin-2-yl)-6-chloro-2,8-difluoroquinazolin-4-yl)-3,8-diazabicyclo[3.2.1]octane-8-carboxylate

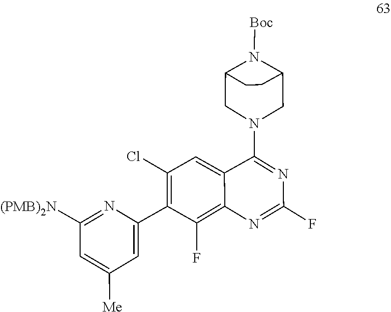

To a degassed solution of tert-butyl-3-(7-bromo-6-chloro-2,8-difluoroquinazolin-4-yl)-3,8-diazabicyclo[3.2.1]octane-8-carboxylate (Intermediate 36, 2.00 g, 4.08 mmol) in anhydrous 1,4-dioxane (20 mL) were added potassium phosphate (1.73 g, 8.17 mmol), N,N-bis(4-methoxybenzyl)-4-methyl-6-(4,4,5,5-tetramethyl-1,3,2-dioxaborolan-2-yl)pyridin-2-amine (5.8 g, 12.25 mmol) and PdCl₂(dppf) (149 mg, 0.204 mmol). The reaction mixture was degassed again and heated at 80° C. for 48 h. After completion of the reaction, the reaction mixture was allowed to cool to ambient temperature, diluted with EtOAc (40 mL), filtered through a bed of a CELITE™ (Sigma Aldrich, St. Louis, MO) and concentrated under reduced pressure to provide crude product. The residue was purified by COMBIFLASH™ chromatography (Teledyne ISO, Lincoln, NE) using 30% EtOAc in petroleum ether to obtain tert-butyl 3-(7-(6-(bis(4-methoxybenzyl)amino)-4-methylpyridin-2-yl)-6-chloro-2,8-difluoroquinazolin-4-yl)-3,8-diazabicyclo[3.2.1]octane-8-carboxylate (1.5 g, 1.74 mmol, 42% yield). ¹H NMR (400 MHz, CDCl₃): δ ppm=7.76 (d, J=1.6 Hz, 1H), 7.20-7.18 (d, J=8.8 Hz, 4H), 6.86 (dt, J=9.6 Hz, 4H), 6.60 (s, 1H), 6.38 (s, 1H), 4.60 (s, 3H), 4.39-4.21 (m, 4H), 3.63 (s, 6H), 2.29 (s, 3H), 1.98-1.96 (m, 6H), 1.76-1.63 (m, 2H), 1.49 (s, 9H) LCMS (ESI) m/z: 757.2 [M+H]⁺.

### Preparation of Intermediate 64: tert-butyl 3-(7-(6-(bis(4-methoxybenzyl)amino)-3-iodo-4-methylpyridin-2-yl)-6-chloro-2,8-difluoroquinazolin-4-yl)-3,8 diazabicyclo[3.2.1]octane-8-carboxylate

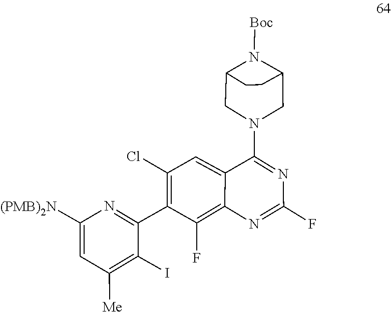

To a stirred solution of tert-butyl 3-(7-(6-(bis(4-methoxybenzyl)amino)-4-methylpyridin-2-yl)-6-chloro-2,8-difluoroquinazolin-4-yl)-3,8-diazabicyclo[3.2.1]octane-8-carboxylate (Intermediate 37, 1.40 g, 1.849 mmol) in anhydrous ACN (15 mL) under nitrogen at 0° C. were added N-iodosuccinimide (0.42 g, 1.849 mmol) and trifluoroacetic acid (0.028 mL, 0.370 mmol). The reaction mixture was allowed to reach room temperature over one hour. The reaction mixture was then quenched with saturated aqueous sodium thiosulfate (5 mL) and saturated aqueous sodium bicarbonate (4 mL). The mixture was extracted with EtOAc (3×20 mL). Combined organic layers were dried over anhydrous sodium sulfate, filtered, and concentrated under vacuum to obtain the crude residue. The crude residue was purified by COMBIFLASH™ chromatography (Teledyne ISO, Lincoln, NE) using 30% EtOAc in petroleum ether to obtain tert-butyl 3-(7-(6-(bis(4-methoxybenzyl)amino)-3-iodo-4-methylpyridin-2-yl)-6-chloro-2,8-difluoroquinazolin-4-yl)-3,8 diazabicyclo[3.2.1]octane-8-carboxylate (1.42 g, 1.560 mmol, 84% yield) as pale yellow fluffy solid. LCMS (ESI) m/z: 883.3 [M+H]⁺.

### Preparation of Intermediate 65: tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate

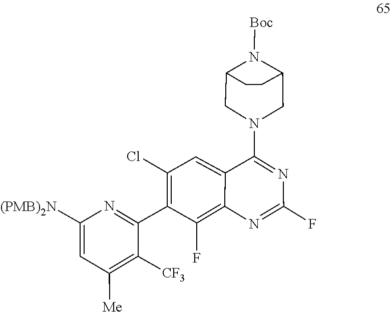

To a stirred solution of tert-butyl 3-(7-(6-(bis(4-methoxybenzyl)amino)-3-iodo-4-methylpyridin-2-yl)-6-chloro-2,8-difluoroquinazolin-4-yl)-3,8-diazabicyclo[3.2.1]octane-8-carboxylate (Intermediate 38, 1.40 g, 1.585 mmol) in anhydrous DMA (10 mL) in a sealed tube under nitrogen was added copper(I) iodide (0.60 g, 3.17 mmol). The reaction mixture was degassed 10 minutes before the addition of methyl 2,2-difluoro-2-(fluorosulfonyl)acetate (0.91 g, 4.76 mmol) and reaction mixture was heated at 90° C. for 12 h. Reaction progress was monitored by LCMS. Reaction mixture was diluted with diethyl ether (20 mL) and water (10 mL). Layers were separated, and the aqueous layer was extracted with diethyl ether (3×20 mL). The combined organic layers were dried over anhydrous sodium sulfate, filtered, and concentrated under reduced pressure to obtain the crude residue. The crude compound was purified by COMBFLASH™ chromatography (Teledyne ISO, Lincoln, NE) using 30% EtOAc in petroleum ether to obtain tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate (0.85 g, 0.630 mmol, 40% yield) as a pale-yellow solid. ¹H NMR (400 MHz, CDCl₃): δ ppm=7.77 (s, 1H), 7.16 (d, J=8.8 Hz, 4H), 6.87 (dt, J=9.6 and 2.8 Hz, 4H), 6.43 (s, 1H), 4.76-4.72 (m, 2H), 4.59-4.55 (m, 2H), 3.81 (s, 6H), 2.43 (s, 3H), 1.97-1.82 (m, 4H), 1.97-1.82 (m, 4H), 1.53 (s, 9H). LCMS (ESI) m/z: 825.2 [M+H]⁺.

### Intermediates 66 and 67: tert-butyl 3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate

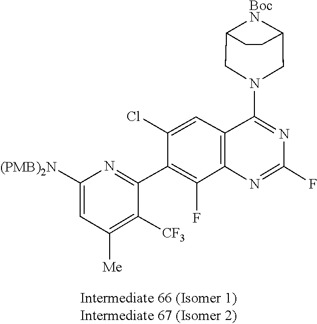

Intermediate 65 (5.0 g, 6.06 mmol) was subjected to SFC separation (Method Information: Column: CHIRALPAK™ IH (Daicel, Japan) (250 mm×4.6 mm; 5 μm), mobile phase—0.25% Isopropanol), where Peak-1 eluted at retention time=5.85 min (2.4 g, 2.90 mmol, 40% yield) and Peak-2 at retention time=9.53 min (2.4 g, 2.90 mmol, 40% yield). Peak-1 (Intermediate 66): ¹H NMR (400 MHz, CDCl₃): δ ppm=7.78 (s, 1H), 7.16 (d, J=8.8 Hz, 4H), 6.87 (dt, J=9.6 and 2.8 Hz, 4H), 6.43 (s, 1H), 4.76-4.72 (m, 2H), 4.59-4.55 (m, 2H), 3.81 (s, 6H), 2.43 (s, 3H), 1.97-1.82 (m, 4H), 1.97-1.82 (m, 4H), 1.53 (s, 9H) ppm. LCMS (ESI) m/z: 825.2 [M+H]⁺. LCMS (ESI) m/z: 825.2 [M+H]⁺. [α]^{23.5} (MeOH=0.10)=+96.00; Peak-2: (Intermediate 67) ¹H NMR (400 MHz, CDCl₃): δ ppm=7.78 (s, 1H), 7.16 (d, J=8.8 Hz, 4H), 6.87 (dt, J=9.6 and 2.8 Hz, 4H), 6.43 (s, 1H), 4.76-4.72 (m, 2H), 4.59-4.55 (m, 2H), 3.81 (s, 6H), 2.43 (s, 3H), 1.97-1.82 (m, 4H), 1.97-1.82 (m, 4H), 1.53 (s, 9H) ppm. LCMS (ESI) m/z: 825.2 [M+H]⁺. [α]²³³ (MeOH=0.10)=−110.00.

### Preparation of Intermediate 68: tert-butyl 3-(2-{[(4aS,7aR)-1-methyl-octahydro-1H-cyclopenta[b]pyridin-4a yl]methoxy}-7-(6-{bis[(4-methoxyphenyl)methyl]amino}-4-methyl-3-(trifluoromethyl)pyridin-2 yl)-6-chloro-8 fluoroquinazolin-4 yl)-3,8-diazabicyclo[3.2.1]octane-8-carboxylate

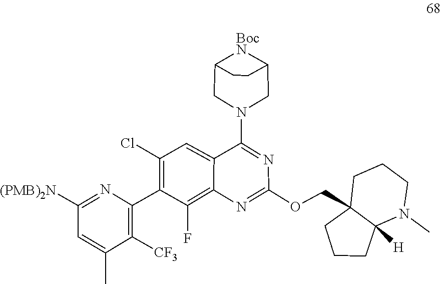

To a solution of [(4aS,7aR)-1-methyl-octahydro-1H-cyclopenta[b]pyridin-4a-yl]methanol (0.205 g, 1.212 mmol, Intermediate 67) in THE (5 mL) at 0° C. was added NaH (0.194 g, 4.85 mmol) and the mixture was stirred at room temperature for 30 minutes. To this was added tert-butyl (1R,5S)-3-(7-(6-(bis(4-methoxybenzyl)amino)-4-methyl-3-(trifluoromethyl)pyridin-2-yl)-6-chloro-2,8-difluoroquinazolin-4-yl)-3,8-diazabicyclo[3.2.1]octane-8-carboxylate (1 g, 1.212 mmol) at 0° C. and mixture was stirred at room temperature for 1 h. The reaction mixture was quenched with ice cubes and concentrated under reduced pressure. The crude was partitioned in between EtOAc and water. The organic layer was dried over sodium sulfate, filtered, and concentrated to provide the crude product, which was purified by COMBIFLASH™ chromatography (Teledyne ISO, Lincoln, NE) (24 g REDISEP™ column (Teledyne ISCO, Lincoln, NE), 0 to 10% MeOH-DCM) to provide tert-butyl 3-(2-{[(4aS,7aR)-1-methyl-octahydro-1H-cyclopenta[b]pyridin-4a-yl]methoxy}-7-(6-{bis[(4-methoxyphenyl)methyl]amino}-4-methyl-3-(trifluoromethyl)pyridin-2-yl)-6-chloro-8-fluoroquinazolin-4-yl)-3,8-diazabicyclo[3.2.1]octane-8-carboxylate (0.70 g, 0.718 mmol, 59% yield). MS(ESI) m/z: 974.4 (M+H)⁺.

### Preparation of Intermediate 69: 6-(2-{[(4aS,7aR)-1-methyl-octahydro-1H-cyclopenta[b]pyridin-4a-yl]methoxy}-6-chloro-4-{3,8-diazabicyclo[3.2.1]octan-3-yl}-8-fluoroquinazolin-7-yl)-N,N-bis[(4-methoxyphenyl)methyl]-4-methyl-5-(trifluoromethyl)pyridin-2-amine

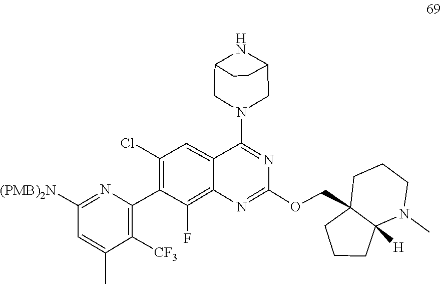

To a solution tert-butyl 3-(2-{[(4aS,7aR)-1-methyl-octahydro-1H-cyclopenta[b]pyridin-4a-yl]methoxy}-7-(6-{bis[(4-methoxyphenyl)methyl]amino}-4-methyl-3-(trifluoromethyl)pyridin-2-yl)-6-chloro-8-fluoroquinazolin-4-yl)-3,8-diazabicyclo[3.2.1]octane-8-carboxylate (700 mg, 0.718 mmol) in acetonitrile (5 mL), was added 4M HCl in dioxane (0.9 mL, 3.59 mmol) at 0° C. The reaction mixture was allowed to warm to room temperature and stirred for 3 h. Then the solvent was removed under reduced pressure, diluted with MeOH, neutralized with excess TEA and again concentrated under reduced pressure to give 6-(2-{[(4aS,7aR)-1-methyl-octahydro-1H-cyclopenta[b]pyridin-4a-yl]methoxy}-6-chloro-4-{3,8-diazabicyclo[3.2.1]octan-3-yl}-8-fluoroquinazolin-7-yl)-N,N-bis[(4-methoxyphenyl)methyl]-4-methyl-5-(trifluoromethyl)pyridin-2-amine (410 mg, 0.47 mmol, 57% yield). MS(ESI) m/z: 874.3 (M+H)+.

### Preparation of Intermediate 70: 6-(2-{[(4aS,7aR)-1-methyl-octahydro-1H-cyclopenta[b]pyridin-4a-yl]methoxy}-6-chloro-4-[8-(3,3-difluorocyclobutanecarbonyl)-3,8-diazabicyclo[3.2.1]octan-3-yl]-8-fluoroquinazolin-7-yl)-N,N-bis[(4-methoxyphenyl)methyl]-4-methyl-5-(trifluoromethyl)pyridin-2-amine

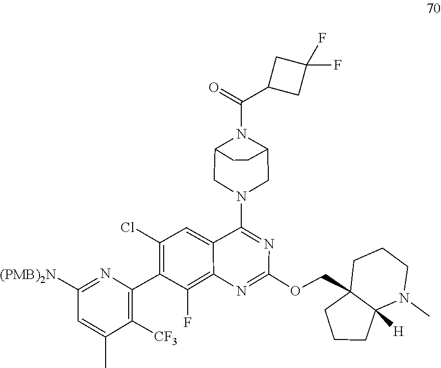

To a stirred solution of 6-(2-{[(4aS,7aR)-1-methyl-octahydro-1H-cyclopenta[b]pyridin-4a-yl]methoxy}-6-chloro-4-{3,8-diazabicyclo[3.2.1]octan-3-yl}-8-fluoroquinazolin-7-yl)-N,N-bis[(4-methoxyphenyl)methyl]-4-methyl-5-(trifluoromethyl)pyridin-2-amine (50 mg, 0.06 mmol) in DCM (10 mL) was added DIPEA (0.03 mL, 0.17 mmol) and propylphosphonic anhydride solution in EtOAc (0.03 mL, 0.11 mmol) at 0° C. After being stirred for 15 min at 0° C., 3,3-difluorocyclobutane-1-carboxylic acid (9.4 mg, 0.07 mmol) was added, and the reaction mixture was allowed to warm to room temperature and stirred for 16 h. The reaction mixture was diluted with EtOAc, washed with water and brine solution, and then dried over sodium sulfate, filtered, and concentrated to give 6-(2-{[(4aS,7aR)-1-methyl-octahydro-1H-cyclopenta[b]pyridin-4a-yl]methoxy}-6-chloro-4-[8-(3,3-difluorocyclobutanecarbonyl)-3,8-diazabicyclo[3.2.1]octan-3-yl]-8-fluoroquinazolin-7-yl)-N,N-bis[(4-methoxyphenyl)methyl]-4-methyl-5-(trifluoromethyl)pyridin-2-amine (54 mg, 0.05 mmol, 95% yield). MS(ESI) m/z: 992.3 (M+H)⁺.

### Example 8-1: 6-(2-{[(4aS,7aR)-1-methyl-octahydro-1H-cyclopenta[b]pyridin-4a-yl]methoxy}-6-chloro-4-[8-(3,3-difluorocyclobutanecarbonyl)-3,8-diazabicyclo[3.2.1]octan-3-yl]-8-fluoroquinazolin-7-yl)-4-methyl-5-(trifluoromethyl)pyridin-2-amine

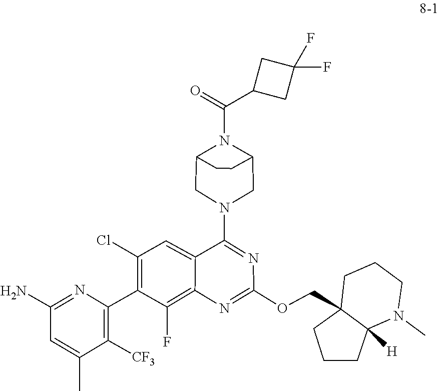 [SMILES: Cc1cc(N)nc(-c2c(Cl)cc3c(N4CC5CCC(C4)N5C(=O)C4CC(F)(F)C4)nc(OC[C@]45CCC[C@H]4N(C)CCC5)nc3c2F)c1C(F)(F)F]

A vial was charged with 6-(2-{[(4aS,7aR)-1-methyl-octahydro-1H-cyclopenta[b]pyridin-4a-yl]methoxy}-6-chloro-4-[8-(3,3-difluorocyclobutanecarbonyl)-3,8-diazabicyclo[3.2.1]octan-3-yl]-8-fluoroquinazolin-7-yl)-N,N-bis[(4-methoxyphenyl)methyl]-4-methyl-5-(trifluoromethyl)pyridin-2-amine (54 mg, 0.054 mmol)), triethylsilane (8.69 μL, 0.054 mmol) and TFA (4.19 μL, 0.054 mmol). Then, the vial was sealed and heated at 40° C. for 16 h. The volatiles from the reaction mixture were removed under reduced pressure and the crude residue was co-evaporated with 1,4-dioxane. Then, the crude residue was dissolved in 1,4-dioxane (2 mL) and excess TEA (1 mL) was added. The mixture was evaporated under reduced pressure to give a crude residue which was purified by prep-HPLC [HPLC Method: Preparative column: Column: Waters XBridge C18, 19×150 mm, 5-μm particles; Mobile Phase A: 0.1% trifluoroacetic acid; Mobile Phase B: acetonitrile; Gradient: 15-47% B over 22 minutes, then a 5-minute hold at 100% B; Flow: 20 mL/min; Detection: UV at 220 &254 nm] to provide 6-(2-{[(4aS,7aR)-1-methyl-octahydro-1H-cyclopenta[b]pyridin-4a-yl]methoxy}-6-chloro-4-[8-(3,3-difluorocyclobutanecarbonyl)-3,8-diazabicyclo[3.2.1]octan-3-yl]-8-fluoroquinazolin-7-yl)-4-methyl-5-(trifluoromethyl)pyridin-2-amine (14.3 mg, 0.018 mmol, 34% yield). MS(electrospray ionization (ESI)) m/z: 752.3 [M+H]⁺.

The example in Table 5 was prepared according to procedures described for Example 8-1 from appropriate starting materials.

TABLE 5

| | | | LCMS |
|---|---|---|---|
| # | Structure | IUPAC | (M + H)⁺ |
| 8-2 | 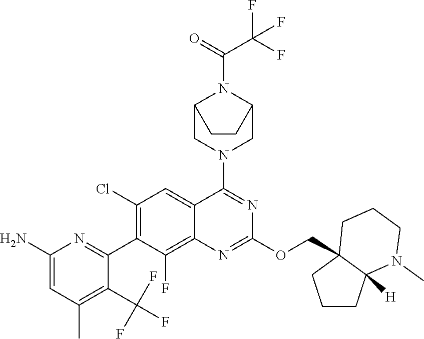 [SMILES: Cc1cc(N)nc(-c2c(Cl)cc3c(N4CC5CCC(C4)N5C(=O)C(F)(F)F)nc(OC[C@]45CCC[C@H]4N(C)CCC5)nc3c2F)c1C(F)(F)F] | 4 1-[3-(2- {[(4aS,7aR)-1- methyl-octahydro- 1H- cyclopenta[b]pyridin- 4a-yl]methoxy}-7-[6- amino-4-methyl-3- (trifluoromethyl)pyri- din-2-yl]-6-chloro-8- fluoroquinazolin-4- yl)-3,8- diazabicyclo[3.2.1]oc- tan-8-yl]-2,2,2- trifluoroethan-1-one | 730.3 |

### Preparation of Intermediate 71: tert-butyl 6-methylene-1,4-oxazepane-4-carboxylate

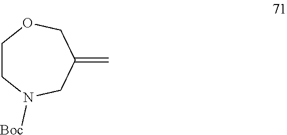

The intermediate tert-butyl 6-methylene-1,4-oxazepane-4-carboxylate was synthesized according to the literature procedure: Bioorg. Med. Chem. Lett. 2019, 29, 2405-2409.

### Preparation of Intermediate 72 tert-butyl 6-oxo-1,4-oxazepane-4-carboxylate

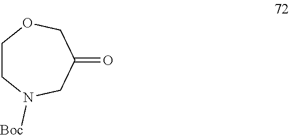 [SMILES: CC(C)(C)OC(=O)N1CCOCC(=O)C1]

To a stirred solution of tert-butyl 6-methylene-1,4-oxazepane-4-carboxylate (6 g, 28.1 mmol) in THE (40 mL) and water (40 mL) was added sodium metaperiodate (12.03 g, 56.3 mmol) followed by an osmium tetroxide solution in tert-butanol (2 mL, 0.141 mmol) at 0° C. The reaction mixture was stirred at room temperature for 16 hours. Then, the reaction mixture was concentrated to afford the crude product. The mixture was extracted with EtOAc (60 mL×3). The combined extracts were washed with brine, dried over anhydrous Na₂SO₄, filtered, and concentrated under reduced pressure to afford a crude residue, which was purified by COMBIFLASH™ chromatography (Teledyne ISO, Lincoln, NE) (pet. ether/EtOAc=30%) to afford the title compound tert-butyl 6-oxo-1,4-oxazepane-4-carboxylate (2 g, 9.29 mmol, 33.0% yield) as a colorless liquid. ¹H NMR (300 MHz, CDCl₃) δ ppm=4.02-4.17 (m, 4H), 3.91 (br d, J=3.78 Hz, 2H), 3.70 (br s, 2H) 1.45 (br s, 9H).

### Preparation of Intermediate 73 and 74: tert-butyl 6-hydroxy-6-methyl-1,4-oxazepane-4-carboxylate

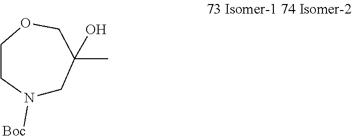 [SMILES: CC1(O)COCCN(C(=O)OC(C)(C)C)C1]

To a stirred solution of tert-butyl 6-oxo-1,4-oxazepane-4-carboxylate (2.5 g, 11.61 mmol) in THE (150 mL), was added methylmagnesium chloride solution in THF (46.5 mL, 46.5 mmol) drop wise at 0° C. The reaction mixture was stirred for 2 h. The reaction mixture was then quenched with saturated NH₄Cl solution and extracted with ethyl acetate (2×100 mL). The combined organic layers were dried over anhydrous Na₂SO₄, filtered, and concentrated under reduced pressure to afford the product. The racemic compound was purified by SFC chiral separation to afford Isomer-1: tert-butyl 6-hydroxy-6-methyl-1,4-oxazepane-4-carboxylate (1 g, 4.32 mmol, 37.2% yield) as and Isomer-2: tert-butyl 6-hydroxy-6-methyl-1,4-oxazepane-4-carboxylate (1 g, 4.32 mmol, 37.2% yield). [Method; Column/dimensions: CHIRALPAK™ IG (Daicel, Japan) (250×50) mm, 5 μm,% CO₂: 60%% Co solvent: 40% OF 4M Methanolic ammonia in MeOH, Total Flow: 280.0 g/min, Back Pressure: 100bar, Temperature: 40° C., UV: 205 nm, RT=4.15 min (isomer-1) & RT=6.01 min (isomer-2)].

Isomer-1: ¹H NMR (400 MHz, DMSO-d₆) δ ppm=4.60-4.66 (m, 1H), 3.48-3.73 (m, 3H), 3.35-3.44 (m, 2H), 3.04-3.20 (m, 3H), 1.41 (s, 9H), 1.02-1.15 (m, 3H).

Isomer-2: ¹H NMR (400 MHz, DMSO-d₆) δ ppm=4.57-4.73 (m, 1H), 3.49-3.77 (m, 3H), 3.35-3.48 (m, 2H), 3.06-3.25 (m, 3H), 1.41 (s, 9H), 1.07-1.08 (m, 3H).

### Preparation of Intermediate 75 and 76: 6-methyl-1,4-oxazepan-6-ol

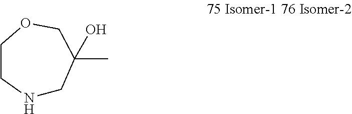

Intermediate 75 (Isomer-1): To a stirred solution of tert-butyl 6-hydroxy-6-methyl-1,4-oxazepane-4-carboxylate (Intermediate 73, 1 g, 4.32 mmol) in acetonitrile (10 mL) was added HCl (4M in dioxane) (5.40 mL, 21.62 mmol). The resulting reaction mixture was stirred at 0° C. for 2 h. The reaction mixture was concentrated and co-evaporated with toluene (two times) to afford 6-methyl-1,4-oxazepan-6-ol hydrochloride (550 mg, 3.28 mmol, 76% yield) as white solid. MS(ESI) m/z: 132.1 [M+H]⁺.

Intermediate 76 (Isomer-2): To a stirred solution of tert-butyl 6-hydroxy-6-methyl-1,4-oxazepane-4-carboxylate (Intermediate 74, 1 g, 4.32 mmol) in acetonitrile (10 mL) was added HCl (4M in dioxane) (5.40 mL, 21.62 mmol). The resulting reaction mixture was stirred at 0° C. for 2 h. The reaction mixture was concentrated and co-evaporated with toluene (two times) to afford 6-methyl-1,4-oxazepan-6-ol hydrochloride (550 mg, 3.28 mmol, 76% yield). MS(ESI) m/z: 132.2 [M+H]⁺.

Intermediate 75 (Isomer-1) was used for the preparation of Examples 1-84, 1-108, 2-70, and 2-74.

### Preparation of Intermediate 77 and 78: tert-butyl 6-(hydroxymethyl)-1,4-oxazepane-4-carboxylate

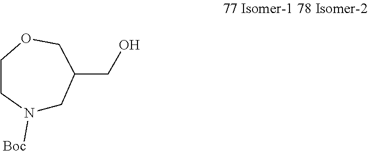 [SMILES: CC(C)(C)OC(=O)N1CCOCC(CO)C1]

To an ice cold THE (15 mL) solution of tert-butyl 6-methylene-1,4-oxazepane-4-carboxylate (1.2 g, 5.63 mmol) was added borane tetrahydrofuran complex solution in THE (6.75 ml, 6.75 mmol). The reaction mixture was heated to 25° C. and stirred for 3 h. Then the reaction mixture was cooled to 0-10° C. and treated sequentially with 3N sodium hydroxide (17 mL) and 30% hydrogen peroxide (6 mL). The obtained homogenous mixture was stirred overnight at room temperature, then treated with hexane (45 mL), and dried over potassium carbonate. The organic layer was decanted from the precipitate, which was washed with dichloromethane. The organic layers were evaporated to afford the title compound. The racemic compound was purified by SFC chiral separation to afford Isomer-1 (Intermediate 77): tert-butyl 6-(hydroxymethyl)-1,4-oxazepane-4-carboxylate (200 mg, 0.865 mmol, 15.37% yield; peak 1), MS(ESI) m/z: 232.1 [M+H]⁺ and Isomer-2 (Intermediate 78): tert-butyl 6-(hydroxymethyl)-1,4-oxazepane-4-carboxylate (220 mg, 0.951 mmol, 16.91% yield; peak 2). MS(ESI) m/z: 132.0 [M-100]⁺. [Method: FlowRate: 3 ml/min, Column Name: CHIRALPAK™ IG (Daicel, Japan) (250×4.6) mm. 5 m, Co-Solvent: 30% Vial No: LD8, Co-Solvent Name: 0.2% of Ammonia Methanol Injected Volume: 20 μl, Outlet Pressure: 100 bar Temperature: 40° C., Comment: SFC-3G-30%-15MTS-205 NM, t_{R}=2.0 min (isomer-1) & t_{R}=4.05 min (isomer-2)].

### Preparation of Intermediate 79 and 80: (1,4-oxazepan-6-yl)methanol

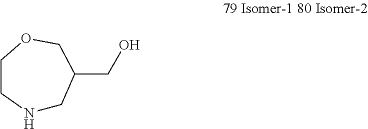

Intermediate 79 (Isomer-1): To a solution of tert-butyl 6-(hydroxymethyl)-1,4-oxazepane-4-carboxylate (Intermediate 77, 200 mg, 0.865 mmol) in acetonitrile (2 mL) at 0° C. was added hydrochloric acid 4.0 M in dioxane (0.026 ml, 0.865 mmol) and the mixture was stirred for 1 h. Then reaction mixture was concentrated and co-evaporated with CAN to afford (1,4-oxazepan-6-yl)methanol hydrochloride (130 mg, 0.776 mmol, 90% yield). MS(ESI) m/z: 132.2 [M+H]⁺.

Intermediate 80 (Isomer-2): To a solution of tert-butyl 6-(hydroxymethyl)-1,4-oxazepane-4-carboxylate (Intermediate 78, 200 mg, 0.865 mmol) in acetonitrile (2 mL) at 0° C. was added hydrochloric acid 4.0M in dioxane (0.026 ml, 0.865 mmol) and the mixture was stirred for 1 h. Then reaction mixture then was concentrated and co-evaporated with CAN to afford (1,4-oxazepan-6-yl)methanol hydrochloride (110 mg, 0.658 mmol, 76% yield) as pale yellow solid. MS(ESI) m/z: 132.1 [M+H]⁺.

Intermediate 80 (Isomer-2) was used for the preparation of Example 1-88.

### Preparation of Intermediate 81: tert-butyl 6,6-difluoro-1,4-oxazepane-4-carboxylate

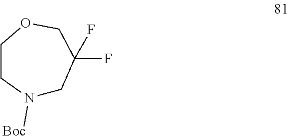 [SMILES: CC(C)(C)OC(=O)N1CCOCC(F)(F)C1]

To a stirred solution of tert-butyl 6-oxo-1,4-oxazepane-4-carboxylate (200 mg, 0.929 mmol) in DCM (4 mL) was added diethylaminosulfur trifluoride (0.982 mL, 7.43 mmol) at 0° C. and the reaction mixture was stirred at 25° C. for 16 h. Then, the reaction mixture was quenched carefully at 0° C. with MeOH followed by water and then concentrated. The crude residue was partitioned between EtOAc and brine. The organic layer was separated, dried over anhydrous Na₂SO₄, filtered, and concentrated to afford tert-butyl 6,6-difluoro-1,4-oxazepane-4-carboxylate (200 mg, 0.843 mmol, 91% yield). ¹H NMR (300 MHz, DMSO-d₆) δ ppm=3.70-3.95 (m, 6H), 3.46-3.57 (m, 2H), 1.40 (s, 9H).

### Preparation of Intermediate 82: 6,6-difluoro-1,4-oxazepane

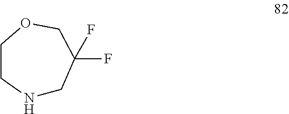

To a stirred solution of tert-butyl 6,6-difluoro-1,4-oxazepane-4-carboxylate (200 mg, 0.843 mmol) in acetonitrile (3 mL) was added HCl 4M in dioxane (0.256 mL, 8.43 mmol) at 0° C. and stirred at room temperature for 1 h. Then reaction mixture was concentrated and co-evaporated with toluene to afford the product 6,6-difluoro-1,4-oxazepane (100 mg, 0.729 mmol, 87% yield). MS(ESI) m/z: 138.0 [M+H]⁺.

### Preparation of Intermediate 83 and 84: 3-methylpiperidin-3-ol

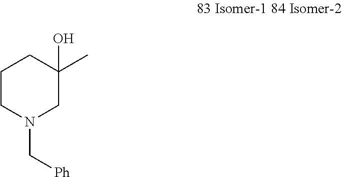

To a stirred solution of 1-benzylpiperidin-3-one (2 g, 10.57 mmol) in tetrahydrofuran (10 mL) was added a methylmagnesium bromide solution in dibutyl ether (14.09 mL, 42.3 mmol) drop wise at 0° C. The reaction mixture was stirred at 0° C. for 2 h. Then, the reaction mixture was slowly quenched with saturated NH₄Cl solution and extracted with ethyl acetate (150 mL×2). The organic layers were washed with brine (˜200 mL) and dried over anhydrous Na₂SO₄, filtered, concentrated under reduced pressure to afford racemic mixture. The racemate mixture was purified by SFC chiral purification to afford 1-benzyl-3-methylpiperidin-3-ol, isomer 1-Intermediate 83 (0.57 g, 2.78 mmol, 26.3% yield) and isomer-2-Intermediate 84 (0.6 g, 2.92 mmol, 27.7% yield). [Method: Column/dimensions: CHIRALPAK™ AD-H (Daicel, Japan) (250×30) mm, 5 μm, % CO₂: 80%% Co solvent: 20% of 0.2% Ammonia in methanol Total Flow: 130.0 g/min Back Pressure: 100 bar Temperature: 40° C., UV: 220 nm, RT; 2.5 min (isomer-1) & 3.4 min (isomer-2)]. For isomer-1: ¹H NMR (300 MHz, DMSO-d₆) δ ppm=7.21-7.36 (m, 5H), 4.16 (s, 1H), 3.31-3.45 (m, 2H), 2.29-2.48 (m, 1H), 2.19 (br d, J=10.20 Hz, 2H), 2.02 (br d, J=10.58 Hz, 1H), 1.61 (br d, J=3.78 Hz, 1H), 1.23-1.53 (m, 3H), 1.10 (s, 3H). For isomer-2: ¹H NMR (300 MHz, DMSO-d₆) δ ppm=7.21-7.34 (m, 5H), 4.16 (s, 1H), 3.32-3.38 (m, 2H), 2.35 (br s, 1H), 2.19 (br d, J=10.58 Hz, 2H), 2.02 (br d, J=10.95 Hz, 1H), 1.61 (br s, 1H), 1.27-1.51 (m, 3H), 1.10 (s, 3H).

### Preparation of Intermediate 85: 3-methylpiperidin-3-ol (Isomer-1)

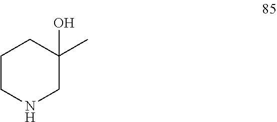

A stirred solution of 1-benzyl-3-methylpiperidin-3-ol (Intermediate 83) (570 mg, 2.78 mmol) in MeOH (10 mL) was purged with nitrogen for 3 minutes and palladium on carbon, 10% (1182 mg, 1.111 mmol) was added. The resulting reaction mixture was stirred at room temperature under a hydrogen balloon for 5 h. The reaction mixture was filtered through a CELITE™ (Sigma Aldrich, St. Louis, MO) pad and washed with MeOH (20 mL). The filtrate was evaporated under reduced pressure to afford 3-methylpiperidin-3-ol (200 mg, 1.736 mmol, 62.5% yield). MS(ESI) m/z: 116.2 [M+H]⁺.

Intermediate 85 was used for the preparation of Examples 1-85, 1-89, 1-95, and 2-72.

### Preparation of Intermediate 86: ethyl 3-(benzylamino)butanoate

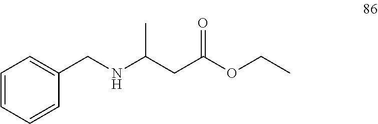 [SMILES: CCOC(=O)CC(C)NCc1ccccc1]

To a stirring solution of benzylamine (5 g, 46.7 mmol) in EtOH (25 mL), was added trans-ethyl crotonate, 96% (6.96 mL, 56.0 mmol) at 0° C. and was allowed to stirred at 90° C. for 16 h. The reaction mixture was then cooled to room temperature and concentrated under reduced pressure to remove volatiles. The crude residue was purified via COMBIFLASH™ chromatography (Teledyne ISO, Lincoln, NE) (2-5% Methanol/DCM, 40 g column) to afford the desired product ethyl 3-(benzylamino)butanoate (7 g, 31.6 mmol, 67.8% yield). MS(ESI) m/z: 222.2 [M+H]⁺.

### Preparation of Intermediate 87: 3-(benzylamino)butan-1-ol

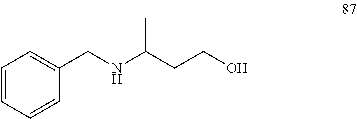

To a suspension of LAH (1.831 g, 48.2 mmol) in THE (100 mL) was added a solution of methyl 3-(benzylamino)butanoate (5 g, 24.12 mmol) in THE (100 mL) dropwise at 0° C. under a nitrogen atmosphere. The resulting mixture was warmed to room temperature and stirred for 3 h. Then, a small amount of saturated aqueous solution of sodium sulfate was added dropwise to quench the reaction at 0° C. and the mixture was stirred for 1 h. The resulting solid material was removed by filtration and the organic solution was concentrated to yield 3-(benzylamino)butan-1-ol (3.9 g, 13.05 mmol, 54.1% yield). ¹H NMR (300 MHz, DMSO-d₆) δ ppm=7.36-7.18 (m, 5H), 3.78-3.69 (m, 1H), 3.63-3.31 (m, 3H), 2.75-2.56 (m, 1H), 2.45-2.15 (m, 1H), 2.11-1.85 (m, 1H), 1.73-1.52 (m, 1H), 1.48-1.33 (m, 1H), 1.01 (d, J=6.4 Hz, 2H), 0.93-0.77 (m, 1H).

### Preparation of Intermediate 88: 4-benzyl-5-methyl-1,4-oxazepan-3-one

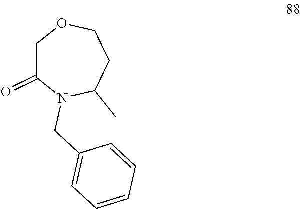 [SMILES: CC1CCOCC(=O)N1Cc1ccccc1]

To a solution of 3-(benzylamino)butan-1-ol (3 g, 16.74 mmol) and triethylamine (4.99 mL, 35.8 mmol) in DCM (50 mL) was added chloroacetyl chloride (1.557 mL, 19.58 mmol) at 0° C. and the mixture was stirred for one hour. The reaction mixture was poured into 1N HCl aqueous solution and extracted with ethyl acetate. The organic layers were dried over anhydrous sodium sulfate, filtered, and concentrated under reduced pressure. The resulting residue was dissolved in 2-propanol (20 mL) and potassium hydroxide (0.66 g, 10 mmol) was added to the solution at room temperature. After stirring for 18 h, the reaction mixture was diluted with water and extracted with ethyl acetate. The organic layer was dried over anhydrous sodium sulfate, filtered, and concentrated under reduced pressure. The crude compound was purified by COMBIFLASH™ chromatography (Teledyne ISO, Lincoln, NE) (hexane/ethyl acetate=100/0 to 1/1 as an eluant) to afford 4-benzyl-5-methyl-1,4-oxazepan-3-one (2.6 g, 10.67 mmol, 63.8% yield). MS(ESI) m/z: 220.2 [M+H]⁺.

### Preparation of Intermediate 89 and 90: 4-benzyl-5-methyl-1,4-oxazepane

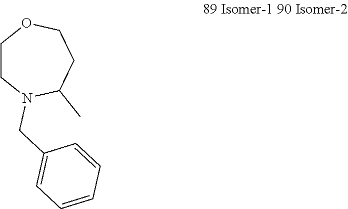

To a solution of 4-benzyl-5-methyl-1,4-oxazepan-3-one (2.64 g, 12.04 mmol) in THE (48 mL) was added borane tetrahydrofuran complex solution in THE (11.52 mL, 120 mmol) dropwise at 0° C. under a nitrogen atmosphere. The mixture was warmed to room temperature and stirred for 3 hours. Then, the reaction was quenched by the addition of methanol and the solution was concentrated under reduced pressure. The residual oil was dissolved in methanol (30 mL) and 1M sodium hydroxide aqueous solution was added at room temperature. The mixture was refluxed for 3 hours, cooled to room temperature, and diluted with water. The resulting solution was extracted with ethyl acetate and the organic layer was dried over anhydrous sodium sulfate, filtered, and concentrated under reduced pressure. The racemic compound was purified by SFC chiral separation to afford Intermediate 89 (Isomer-1): 4-benzyl-5-methyl-1,4-oxazepane (350 mg, 1.7 mmol, 29.16%) as a pale yellow liquid and Intermediate 90 (Isomer-2): 4-benzyl-5-methyl-1,4-oxazepane (350 mg, 1.7 mmol, 29.16%) as a pale yellow liquid. [Method Column Name: CHIRALPAK™ IG (Daicel, Japan) (250×4.6) mm, 5 m, Co-Solvent Name: 0.2% ammonia in methanol, Flow Rate: 3 ml/min, Co-Solvent Percentage: 10%, Back Pressure: 100, Isomer-1 (RT=3.07 min) & Isomer-2 (RT=3.81 min)] MS(ESI) m/z: 206.2 [M+H]⁺.

### Preparation of Intermediate 91 and 92: 5-methyl-1,4-oxazepane

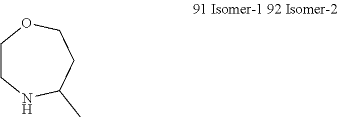

Intermediate 91 (Isomer-1): To a stirred solution of 4-benzyl-5-methyl-1,4-oxazepane (350 mg, 1.705 mmol) in 1,2-dichloroethane (5 mL) was added 1-chloroethyl chloroformate (2437 mg, 17.05 mmol) at room temperature. Reaction mixture was stirred at 90° C. for 16 h. Reaction mixture was cooled to room temperature and concentrated completely. Crude mixture was redissolved in MeOH (5 mL) and refluxed for 1 h. The reaction mixture was cooled to room temperature and concentrated completely to afford 5-methyl-1,4-oxazepane (150 mg, 1.302 mmol, 76% yield) as a gummy liquid. MS(ESI) m/z: 116.2 [M+H]⁺.

Intermediate 92 (Isomer-2): To a stirred solution of 4-benzyl-5-methyl-1,4-oxazepane (350 mg, 1.705 mmol) in 1,2-dichloroethane (5 mL) was added 1-chloroethyl chloroformate (2437 mg, 17.05 mmol) at room temperature. Reaction mixture was stirred at 90° C. for 16 h. Reaction mixture was cooled to room temperature and concentrated completely. Crude was dissolved in MeOH (5 mL) and refluxed for 1 h. Reaction mixture was cooled to room temperature and concentrated completely to afford 5-methyl-1,4-oxazepane (140 mg, 1.216 mmol, 71.3% yield) as a gummy liquid. MS(ESI) m/z: 116.1 [M+H]+.

Intermediate 91 (Isomer-1) was used for the preparation of Examples 1-93 and 1-96.

### Preparation of Intermediate 93 and 94: tert-butyl 1-methyl-4,6-dihydropyrrolo[3,4-c]pyrazole-5(1H)-carboxylate and tert-butyl 2-methyl-2,6-dihydropyrrolo[3,4-c]pyrazole-5(4H)-carboxylate

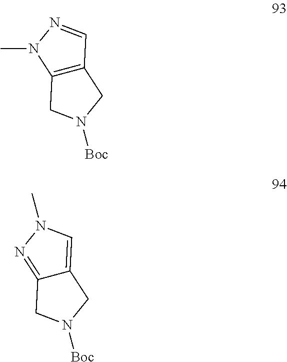 [SMILES: Cn1ncc2c1CN(C(=O)OC(C)(C)C)C2 ; Cn1cc2c(n1)CN(C(=O)OC(C)(C)C)C2]

To a stirred solution of 4,6-dihydro-1h-pyrrolo[3,4-c]pyrazole-5-carboxylic acid tert-butyl ester (2 g, 9.56 mmol) in DMF (20 mL) was added cesium carbonate (6.23 g, 19.12 mmol) and iodomethane (1.195 mL, 19.12 mmol) at room temperature and the mixture was stirred for 16 h. Then the reaction mixture was poured into water and extracted with EtOAc (50×3). The organic layer was washed with brine solution, dried over anhydrous Na₂SO₄, filtered, and concentrated under reduced pressure to afford a mixture of regioisomers. The mixture of regioisomers were separated by SFC [Conditions Column/dimensions: CHIRALPAK™ OZ-H (Daicel, Japan) (250×30) mm, 5 μm, % CO₂: 90%. % Co solvent: 10% of 5 mM ammonium acetate in ACN:MeOH (50:50) Total Flow: 150.0 g/min Back Pressure: 100 bar Temperature: 40° C. UV: 220 nm] Regioisomer-1 (Intermediate 93) (t_{R}=3 min) (0.2 g, 0.896 mmol, 9.37% yield) Regioisomer-2 (Intermediate 94) (t_{R}=4.37 min)] (0.8 g, 3.58 mmol, 37.5% yield, regioisomer-2).

Intermediate 93, tert-butyl 1-methyl-4,6-dihydropyrrolo[3,4-c]pyrazole-5(1H)-carboxylate: ¹H NMR (400 MHz, CDCl₃) δ ppm=7.28-7.20 (m, 1H), 4.62-4.30 (m, 4H), 3.84 (d, J=2.5 Hz, 3H), 1.53 (d, J=2.5 Hz, 9H).

Intermediate 94, tert-butyl 2-methyl-2,6-dihydropyrrolo[3,4-c]pyrazole-5(4H)-carboxylate: ¹H NMR (400 MHz, CDCl₃) δ ppm=7.22-7.04 (m, 1H), 4.63-4.31 (m, 4H), 3.92 (s, 3H), 1.53 (d, J=2.0 Hz, 9H).

### Preparation of Intermediate 95: 1-methyl-1,4,5,6-tetrahydropyrrolo[3,4-c]pyrazole

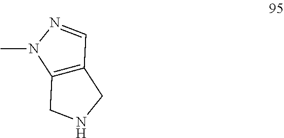

To a stirred solution of tert-butyl 1-methyl-4,6-dihydropyrrolo[3,4-c]pyrazole-5(1H)-carboxylate (100 mg, 0.448 mmol) in acetonitrile (5 mL) at 0° C. was added HCl (4M in dioxane) (1.120 mL, 4.48 mmol) and the reaction mixture was stirred at 0° C. for 1 h. The volatiles from the reaction mixture were removed under reduced pressure to afford 1-methyl-1,4,5,6-tetrahydropyrrolo[3,4-c]pyrazole hydrochloride salt (50 mg, 0.406 mmol, 91% yield) as an off-white solid. MS(ESI) m/z: 124.1 [M+H]⁺.

### Preparation of Intermediate 96: 2-methyl-2,4,5,6-tetrahydropyrrolo[3,4-c]pyrazole

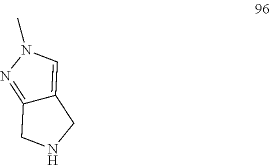

To a stirred solution of tert-butyl 2-methyl-2,6-dihydropyrrolo[3,4-c]pyrazole-5(4H)-carboxylate (100 mg, 0.448 mmol) in acetonitrile (2 mL) at 0° C. was added HCl (4M in dioxane) (1.120 mL, 4.48 mmol) and the reaction mixture was stirred at 0° C. for 1 h. The volatiles from the reaction mixture were removed under reduced pressure to afford 2-methyl-2,4,5,6-tetrahydropyrrolo[3,4-c]pyrazole hydrochloride salt (45 mg, 0.365 mmol, 82% yield) as colorless liquid. MS(ESI) m/z: 124.2 [M+H]⁺.

### Preparation of Intermediate 97: methyl 4-amino-6-chloro-2-methoxypyridine-3-carboxylate

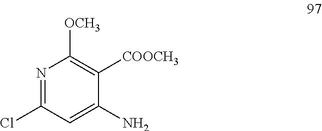 [SMILES: COC(=O)c1c(N)cc(Cl)nc1OC]

To a stirred solution of 6-chloro-3-iodo-2-methoxypyridin-4-amine (prepared as described in WO 2010/093849 and WO 2022/040267) (25 g, 88 mmol) in MeOH (450 mL) was added triethylamine (36.7 mL, 264 mmol) and dppf (4.87 g, 8.79 mmol), followed by palladium(II) acetate (0.986 g, 4.39 mmol). The reaction mixture was purged with N₂ for 5 min, and the reaction mixture was stirred at 60° C. under 2.5 kg of CO gas pressure in an autoclave for 16 h. The reaction mixture was cooled to room temperature, filtered through a CELITE™ (Sigma Aldrich, St. Louis, MO) pad and the pad was washed with methanol. The filtrate was concentrated under reduced pressure to provide the crude product, which was purified by COMBIFLASH™ chromatography (Teledyne ISO, Lincoln, NE) (using 120 g silica gel column, using 30 to 35% ethyl acetate/petroleum ether) to provide methyl 4-amino-6-chloro-2-methoxypyridine-3-carboxylate (16 g, 73.9 mmol, 84% yield) as a white solid. MS(ESI) m/z: 217.1 [M+H]⁺.

### Preparation of Intermediate 98: methyl 4-amino-6-chloro-5-fluoro-2-methoxypyridine-3-carboxylate

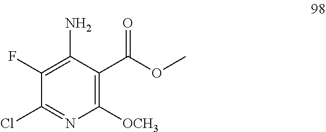

To a stirred solution of methyl 4-amino-6-chloro-2-methoxypyridine-3-carboxylate (19 g, 88 mmol) in ACN (400 mL) was added SELECTFLUOR™ (Air Products, Allentown, PA) (1-chloromethyl-4-fluoro-1,4-diazoniabicyclo[2.2.2]octane bis(tetrafluoroborate)) (31.1 g, 88 mmol). The reaction mixture was stirred at room temperature for 16 h. The reaction mixture was then evaporated under reduced pressure to give the crude product. The crude compound was purified by COMBIFLASH™ chromatography (Teledyne ISO, Lincoln, NE) (120 g silica gel column, using 0 to 10% ethyl acetate/petroleum ether) to provide methyl 4-amino-6-chloro-5-fluoro-2-methoxypyridine-3-carboxylate (3 g, 11.95 mmol, 13.62% yield) as a white solid. MS(ESI) m/z: 235.1 [M+H]⁺.

### Preparation of Intermediate 99: methyl 6-chloro-5-fluoro-2-methoxy-4-{[(2,2,2-trichloroacetyl)carbamoyl]amino}pyridine-3-carboxylate

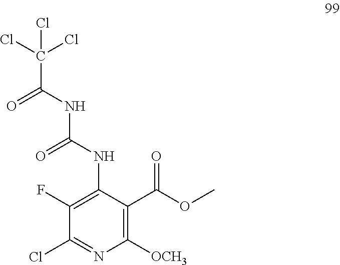 [SMILES: COC(=O)c1c(OC)nc(Cl)c(F)c1NC(=O)NC(=O)C(Cl)(Cl)Cl]

To a stirred solution of methyl 4-amino-6-chloro-5-fluoro-2-methoxypyridine-3-carboxylate (9.5 g, 40.5 mmol) in THE (150 mL) was added trichloroacetyl isocyanate (6.28 mL, 52.6 mmol). The reaction mixture was stirred at room temperature for 2 h. The reaction mixture was concentrated under reduced pressure to provide methyl 6-chloro-5-fluoro-2-methoxy-4-{[(2,2,2-trichloroacetyl)carbamoyl]amino}pyridine-3-carboxylate (17.84 g, 40.5 mmol, 100% yield) as a white solid, which was taken forward without further purification. MS(ESI) m/z: 424.1 [M+H]⁺.

### Preparation of Intermediate 100: 7-chloro-8-fluoro-5-methoxypyrido[4,3-d]pyrimidine-2,4-diol

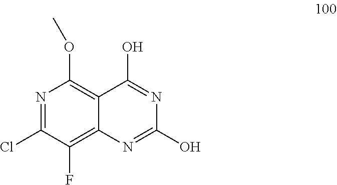 [SMILES: COc1nc(Cl)c(F)c2nc(O)nc(O)c12]

To a stirred solution of methyl 6-chloro-5-fluoro-2-methoxy-4-{[(2,2,2-trichloroacetyl)carbamoyl]amino}pyridine-3-carboxylate (16 g, 37.8 mmol) in MeOH (200 mL) was added a 7N solution of ammonia (270 mL, 1891 mmol, in methanol), slowly over a period of 30 minutes. The reaction mixture was stirred at room temperature for 2 h. The reaction mixture was filtered, and the solid was washed with methanol (200 mL) and dried under vacuum to provide 7-chloro-8-fluoro-5-methoxypyrido[4,3-d]pyrimidine-2,4-diol (9 g, 36.3 mmol, 96% yield) as a white solid, which was taken forward without further purification. MS(ESI) m/z: 246.1 [M+H]⁺.

### Preparation of Intermediate 101: 2,4,7-trichloro-8-fluoro-5-methoxypyrido[4,3-d]pyrimidine

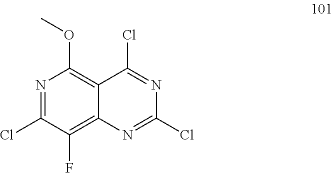 [SMILES: COc1nc(Cl)c(F)c2nc(Cl)nc(Cl)c12]

POCl₃ (133 mL, 1425 mmol) followed by DIPEA (24.89 mL, 143 mmol) was added to 7-chloro-8-fluoro-5-methoxypyrido[4,3-d]pyrimidine-2,4-diol (7 g, 28.5 mmol). The reaction mixture was heated at 90° C. for 2 h. The reaction mixture was then evaporated, and the residue was dissolved in ethyl acetate. This was added to a cooled saturated solution of sodium bicarbonate and stirred for 10 minutes. The organic layer was washed with brine, dried over anhydrous sodium sulfate, filtered, and concentrated under reduced pressure to provide 2,4,7-trichloro-8-fluoro-5-methoxypyrido[4,3-d]pyrimidine (8.05 g, 28.5 mmol, 100% yield) as a pale yellow solid. MS(ESI) m/z: 284.0 [M+H]⁺.

### Preparation of Intermediate 102: 4-{2,7-dichloro-8-fluoro-5-methoxypyrido[4,3-d]pyrimidin-4-yl}-1,4-oxazepane

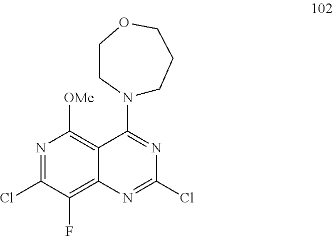 [SMILES: COc1nc(Cl)c(F)c2nc(Cl)nc(N3CCCOCC3)c12]

To a stirred solution of 2,4,7-trichloro-8-fluoro-5-methoxypyrido[4,3-d]pyrimidine (1.5 g, 5.31 mmol) in DCM (20 mL) at −40° C. was added DIPEA (1.85 mL, 10.62 mmol) followed by 1,4-oxazepane (0.537 g, 5.31 mmol). The reaction mixture was slowly brought to room temperature and stirred for 30 minutes. The reaction mixture was treated with water and extracted with DCM. The combined organic layers were washed with brine, dried over anhydrous sodium sulfate, filtered, and evaporated under reduced pressure to give the crude product. The crude compound was purified by COMBIFLASH™ chromatography (Teledyne ISO, Lincoln, NE) (40 g silica gel column, using 50 to 80% ethyl acetate/pet ether) to provide 4-{2,7-dichloro-8-fluoro-5-methoxypyrido[4,3-d] pyrimidin-4-yl}-1,4-oxazepane (1.2 g, 3.46 mmol, 65.1% yield) as a pale-yellow solid. MS(ESI) m/z: 347.0 [M+H]⁺.

### Preparation of Intermediate 103: (2R,7aS)-7a-({[7-chloro-8-fluoro-5-methoxy-4-(1,4-oxazepan-4-yl)pyrido[4,3-d]pyrimidin-2-yl]oxy}methyl)-2-fluoro-hexahydro-1H-pyrrolizine

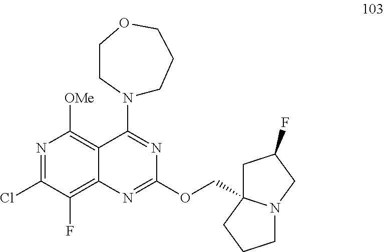 [SMILES: COc1nc(Cl)c(F)c2nc(OC[C@@]34CCCN3C[C@H](F)C4)nc(N3CCCOCC3)c12]

To a solution of [(2R,7aS)-2-fluoro-hexahydro-1H-pyrrolizin-7a-yl]methanol (220 mg, 1.383 mmol) and 4-{2,7-dichloro-8-fluoro-5-methoxypyrido[4,3-d] pyrimidin-4-yl}-1,4-oxazepane (400 mg, 1.152 mmol) in THE (5 mL) was added LiHMDS (1M in THF) (3.46 mL, 3.46 mmol) at 0° C., and the reaction mixture was stirred for 1 h. The reaction mixture was then quenched with saturated ammonium chloride solution and extracted with ethyl acetate. The organic layer was washed with water and brine, and then dried over anhydrous sodium sulfate. The mixture was filtered, and the filtrate was evaporated to provide the crude residue, which was purified by silica gel column chromatography using CombiFlash chromatography (24 g REDISEP™ column (Teledyne ISCO, Lincoln, NE), 80 to 100% EtOAc-petroleum ether) to provide (2R,7aS)-7a-({[7-chloro-8-fluoro-5-methoxy-4-(1,4-oxazepan-4-yl)pyrido[4,3-d]pyrimidin-2-yl]oxy}methyl)-2-fluoro-hexahydro-1H-pyrrolizine (400 mg, 0.851 mmol, 73.9% yield) as a brown solid. MS(ESI) m/z: 470.1 (M+H)⁺.

### Preparation of Intermediate 104: (2R,7aS)-2-fluoro-7a-[({8-fluoro-7-[7-fluoro-3-(methoxymethoxy)-8-{2-[tris(propan-2-yl)silyl]ethynyl}naphthalen-1-yl]-5-methoxy-4-(1,4-oxazepan-4-yl)pyrido[4,3-d]pyrimidin-2-yl}oxy)methyl]-hexahydro-1H-pyrrolizine

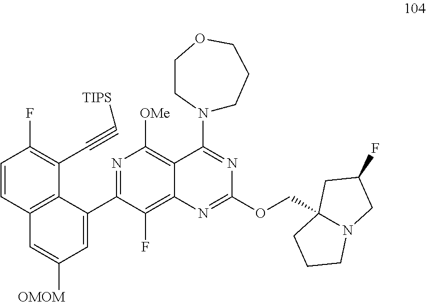

To a stirred solution of (2R,7aS)-7a-({[7-chloro-8-fluoro-5-methoxy-4-(1,4-oxazepan-4-yl)pyrido[4,3-d]pyrimidin-2-yl]oxy}methyl)-2-fluoro-hexahydro-1H-pyrrolizine (400 mg, 0.851 mmol) in 1,4-dioxane (11 mL) and water (3.5 mL) was added ((2-fluoro-6-(methoxymethoxy)-8-(4,4,5,5-tetramethyl-1,3,2-dioxaborolan-2-yl)naphthalen-1-yl)ethynyl)triisopropylsilane (436 mg, 0.851 mmol) and Cs₂CO₃ (832 mg, 2.55 mmol) followed by PdCl₂(dppf) (62.3 mg, 0.085 mmol). The reaction mixture was purged with N2 for 5 min and heated at 105° C. for 1 h in a microwave reactor. The reaction mixture was cooled to room temperature, diluted with water, and extracted with ethyl acetate. The combined organic layers were washed with brine, dried over anhydrous sodium sulfate, filtered, and concentrated under reduced pressure to provide the crude product, which was purified by COMBIFLASH™ chromatography (Teledyne ISO, Lincoln, NE) (using 24 g silica gel column, using 50 to 100% ethyl acetate/petroleum ether) to provide (2R,7aS)-2-fluoro-7a-[({8-fluoro-7-[7-fluoro-3-(methoxymethoxy)-8-{2-[tris(propan-2-yl)silyl]ethynyl}naphthalen-1-yl]-5-methoxy-4-(1,4-oxazepan-4-yl)pyrido[4,3-d]pyrimidin-2-yl}oxy)methyl]-hexahydro-1H-pyrrolizine (220 mg, 0.268 mmol, 31.5% yield) as a brown solid. MS(ESI) m/z: 820.3 [M+H]⁺.

### Preparation of Intermediate 105: (2R,7aS)-7a-[({7-[8-ethynyl-7-fluoro-3-(methoxymethoxy)naphthalen-1-yl]-8-fluoro-5-methoxy-4-(1,4-oxazepan-4-yl)pyrido[4,3-d]pyrimidin-2-yl}oxy)methyl]-2-fluoro-hexahydro-1H-pyrrolizine

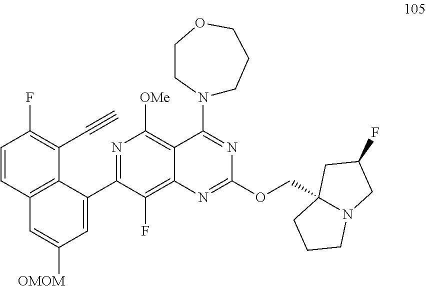

To a stirred solution of (2R,7aS)-2-fluoro-7a-[({8-fluoro-7-[7-fluoro-3-(methoxymethoxy)-8-{2-[tris(propan-2-yl)silyl]ethynyl}naphthalen-1-yl]-5-methoxy-4-(1,4-oxazepan-4-yl)pyrido[4,3-d]pyrimidin-2-yl}oxy)methyl]-hexahydro-1H-pyrrolizine (220 mg, 0.268 mmol) in DMF (3 mL) was added CsF (408 mg, 2.68 mmol). The reaction mixture was stirred at room temperature for 1 h. The reaction mixture was then diluted with water and extracted with ethyl acetate. The combined organic layers were washed with brine, dried over anhydrous sodium sulfate, filtered, and concentrated under reduced pressure to provide (2R,7aS)-7a-[({7-[8-ethynyl-7-fluoro-3-(methoxymethoxy)naphthalen-1-yl]-8-fluoro-5-methoxy-4-(1,4-oxazepan-4-yl)pyrido[4,3-d]pyrimidin-2-yl}oxy)methyl]-2-fluoro-hexahydro-1H-pyrrolizine (130 mg, 0.196 mmol, 73.0% yield) as a brown liquid, which was taken forward without further purification. MS(ESI) m/z: 664.3 [M+H]⁺.

### Example 9-1: 4-(2-{[(2R,7aS)-2-fluoro-hexahydro-1H-pyrrolizin-7a-yl]methoxy}-8-fluoro-5-methoxy-4-(1,4-oxazepan-4-yl)pyrido[4,3-d]pyrimidin-7-yl)-5-ethynyl-6-fluoronaphthalen-2-ol

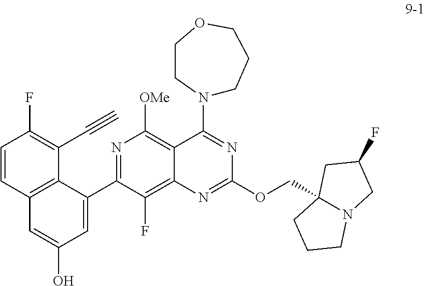 [SMILES: C#Cc1c(F)ccc2cc(O)cc(-c3nc(OC)c4c(N5CCCOCC5)nc(OC[C@@]56CCCN5C[C@H](F)C6)nc4c3F)c12]

To a stirred solution of (2R,7aS)-7a-[({7-[8-ethynyl-7-fluoro-3-(methoxymethoxy)naphthalen-1-yl]-8-fluoro-5-methoxy-4-(1,4-oxazepan-4-yl)pyrido[4,3-d]pyrimidin-2-yl}oxy)methyl]-2-fluoro-hexahydro-1H-pyrrolizine (130 mg, 0.196 mmol) in acetonitrile (2 mL) at 0° C. was added 4N HCl (2 mL, 65.8 mmol in 1,4-dioxane), and the reaction mixture was stirred at room temperature for 1 h. The volatiles from the reaction mixture were removed under reduced pressure (at lower temperature, ˜30° C.), and the crude residue was co-evaporated with 1,4-dioxane. Then the crude residue was dissolved in 1,4-dioxane (2 mL) and excess TEA (1 mL) was added and evaporated under reduced pressure to give an off-white solid. The crude compound was purified by Prep-HPLC [HPLC Method: Preparative Column: YMC Triart exrs C18 (250 mm×20 mm) 5 ID; Mobile Phase A: 10 mM ammonium bicarbonate in water pH 9.5; Mobile Phase B: Acetonitrile:MeOH(1:1); Temperature: 50° C.; Gradient: 50-100% B over 17 minutes; Flow: 19 ml/min. to provide 4-(2-{[(2R,7aS)-2-fluoro-hexahydro-1H-pyrrolizin-7a-yl]methoxy}-8-fluoro-5-methoxy-4-(1,4-oxazepan-4-yl)pyrido[4,3-d]pyrimidin-7-yl)-5-ethynyl-6-fluoronaphthalen-2-ol (27 mg, 0.042 mmol, 21.36% yield). MS(ESI) m/z: 620.3 [M+H]⁺. ¹H NMR (400 MHz, DMSO-d₆) δ ppm=7.92 (dd, J=9.2, 5.9 Hz, 1H), 7.42 (t, J=9.0 Hz, 1H), 7.32 (d, J=2.4 Hz, 1H), 7.20 (d, J=2.1 Hz, 1H), 5.38-5.19 (m, 1H), 4.10 (dd, J=10.4, 5.9 Hz, 1H), 4.04-3.97 (m, 2H), 3.88 (s, 3H), 3.83 (br s, 5H), 3.75-3.69 (m, 2H), 3.13-3.06 (m, 2H), 3.01 (s, 1H), 2.87-2.79 (m, 1H), 2.13 (br d, J=4.3 Hz, 1H), 2.08-1.99 (m, 4H), 1.94 (s, 1H), 1.89-1.75 (m, 3H).

The compound in Table 6 was prepared according to procedures described for Example 9-1 from appropriate starting materials.

TABLE 6

| | | | LCMS | |
|---|---|---|---|---|
| # | Structure | IUPAC | (M + H)⁺ | ¹H NMR |
| 9-2 | 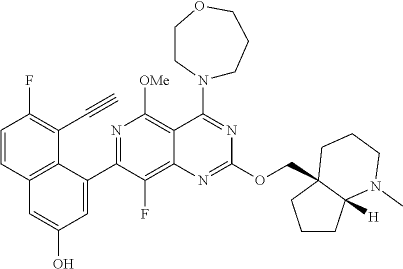 [SMILES: C#Cc1c(F)ccc2cc(O)cc(-c3nc(OC)c4c(N5CCCOCC5)nc(OC[C@]56CCC[C@H]5N(C)CCC6)nc4c3F)c12] | 4-(2- {[(4aS,7aR)-1- methyl- octahydro-1H- cyclopenta[b]pyr- idin-4a- yl]methoxy}-8- fluoro-5- methoxy-4-(1,4- oxazepan-4- yl)pyrido[4,3- d]pyrimidin-7- yl)-5-ethynyl-6- fluoronaphthalen- 2-ol | 630.3 | ¹H NMR (400 MHz, DMSO- d₆) δ ppm = 11.06 - 8.88 (m, 1H), 7.95 (dd, J = 9.1, 6.0 Hz, 1H), 7.45 (t, J = 9.0 Hz, 1H), 7.36 (d, J = 2.5 Hz, 1H), 7.22 (d, J = 2.4 Hz, 1H), 4.42 (d, J = 10.8 Hz, 1H), 4.13 (dd, J = 10.6, 3.6 Hz, 1H), 4.00 (d, J = 0.9 Hz, 1H), 3.93 - 3.79 (m, 9H), 3.73 (dt, J = 6.7, 3.2 Hz, 2H), 2.70 - 2.62 (m, 2H), 2.46 (br s, 1H), 2.26 - 2.15 (m, 4H), 2.08 - 2.01 (m, 2H), 1.87 - 1.67 (m, 2H), 1.65 - 1.50 (m, 6H), 1.43 - 1.35 (m, 1H). |
| 9-3 | 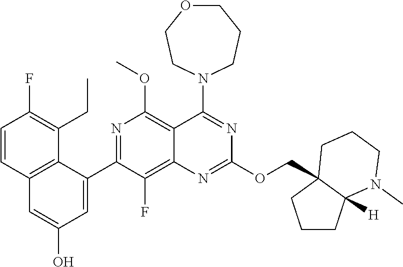 [SMILES: CCc1c(F)ccc2cc(O)cc(-c3nc(OC)c4c(N5CCCOCC5)nc(OC[C@]56CCC[C@H]5N(C)CCC6)nc4c3F)c12] | 4-(2- {[(4aS,7aR)-1- methyl- octahydro-1H- cyclopenta[b]pyr- idin-4a- yl]methoxy}-8- fluoro-5- methoxy-4-(1,4- oxazepan-4- yl)pyrido[4,3- d]pyrimidin-7- yl)-5-ethyl-6- fluoronaphthalen- 2-ol | 634.3 | ¹H NMR (400 MHz, DMSO- d₆) δ ppm = 9.94 (s, 1H), 7.76 (dd, J = 9.1, 6.0 Hz, 1H), 7.38 - 7.29 (m, 2H), 7.07 (d, J = 2.5 Hz, 1H), 4.45 (dd, J = 10.6, 8.4 Hz, 1H), 4.15 (dd, J = 10.6, 8.4 Hz, 1H), 3.96 - 3.78 (m, 11H), 3.74 - 3.69 (m, 2H), 2.71 - 2.66 (m, 1H), 2.44 - 2.38 (m, 1H), 2.20 - 2.14 (m, 1H), 2.13 (s, 3H), 2.08 - 1.96 (m, 2H), 1.88 - 1.69 (m, 2H), 1.66 - 1.49 (m,7H), 1.44 - 1.35 (m, 1H), 0.80 (t, J = 7.3 Hz, 3H). |
| 9-4 | 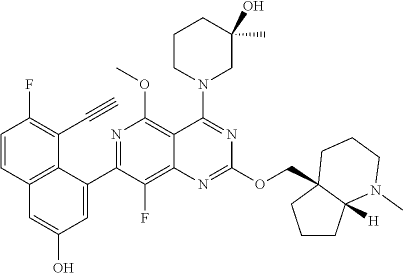 [SMILES: C#Cc1c(F)ccc2cc(O)cc(-c3nc(OC)c4c(N5CCC[C@@](C)(O)C5)nc(OC[C@]56CCC[C@H]5N(C)CCC6)nc4c3F)c12] | (3R)-1-(2- {[(4aS,7aR)-1- methyl- octahydro-1H- cyclopenta[b]pyr- idin-4a- yl]methoxy}-7- (8-ethynyl-7- fluoro-3- hydroxynaphtha- len-1-yl)-8- fluoro-5- methoxypyrido[4, 3-d]pyrimidin- 4-yl)-3- methylpiperidin- 3-ol | 644.3 | ¹H NMR (400 MHz, DMSO- d₆) δ ppm = 10.34 - 9.97 (m, 1H), 7.96 (dd, J = 9.2, 5.9 Hz, 1H), 7.50 - 7.42 (m, 1H), 7.38 (d, J = 2.5 Hz, 1H), 7.30 - 7.20 (m, 1H), 4.61 - 4.35 (m, 3H), 4.16 - 3.95 (m, 2H), 3.91 - 3.85 (m, 3H), 3.57 - 3.40 (m, 2H), 3.07 (br s, 1H), 2.64 (br t, J = 6.4 Hz, 1H), 2.47 - 2.42 (m, 1H), 2.27 - 2.14 (m, 4H), 2.07 - 1.87 (m, 1H), 1.85 - 1.45 (m, 12H), 1.43 - 1.35 (m, 1H), 1.14 - 1.03 (m, 3H). |
| 9-5 | 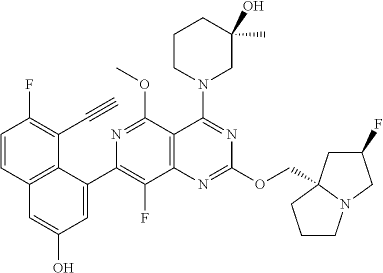 [SMILES: C#Cc1c(F)ccc2cc(O)cc(-c3nc(OC)c4c(N5CCC[C@@](C)(O)C5)nc(OC[C@@]56CCCN5C[C@H](F)C6)nc4c3F)c12] | (3R)-1-(2- {[(2R,7aS)-2- fluoro- hexahydro-1H- pyrrolizin-7a- yl]methoxy}-7- (8-ethynyl-7- fluoro-3- hydroxynaphtha- len-1-yl)-8- fluoro-5- methoxypyrido[4, 3-d]pyrimidin- 4-yl)-3- methylpiperidin- 3-ol | 634.3 | ¹H NMR (400 MHz, DMSO- d₆) δ ppm = 10.40 - 9.86 (m, 1H), 7.97 (dd, J = 9.2, 5.9 Hz, 1H), 7.50 - 7.42 (m, 1H), 7.39 (d, J = 2.5 Hz, 1H), 7.29 - 7.20 (m, 1H), 5.38 - 5.19 (m, 1H), 4.61 - 4.36 (m, 2H), 4.16 - 4.09 (m, 2H), 4.02 - 3.94 (m, 1H), 3.91 - 3.86 (m, 3H), 3.68 (br d, J = 13.1 Hz, 1H), 3.54 - 3.42 (m, 2H), 3.15 - 3.00 (m, 4H), 2.87 - 2.79 (m, 1H), 2.20 - 1.96 (m, 3H), 1.92 - 1.73 (m, 3H), 1.70 - 1.51 (m, 3H), 1.14 - 1.03 (m, 3H). |
| 9-6 | 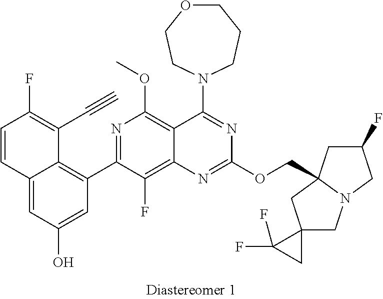 [SMILES: C#Cc1c(F)ccc2cc(O)cc(-c3nc(OC)c4c(N5CCCOCC5)nc(OC[C@@]56C[C@@H](F)CN5CC5(CC5(F)F)C6)nc4c3F)c12] | 5-ethynyl-6- fluoro-4-(8- fluoro-5- methoxy-4-(1,4- oxazepan-4-yl)- 2-(((6′R, 7a′R)- 2,2,6′- trifluorodihydro- 1′H,3′H- spiro[cyclopropane- 1,2′- pyrrolizin]- 7a′(5′H)- yl)methoxy)pyrido [4,3- d]pyrimidin-7- yl)naphthalen-2- ol | 682.2 | ¹H NMR (400 MHz, DMSO- d₆) δ ppm = 10.19 (br s, 1H), 7.97 (dd, J = 9.3, 5.8 Hz, 1H), 7.46 (t, J = 9.0 Hz, 1H), 7.39 (d, J = 2.5 Hz, 1H), 7.29 - 7.15 (m, 1H), 5.52 - 5.30 (m, 1H), 4.18 - 4.13 (m, 1H), 4.05 - 3.96 (m, 2H), 3.93 - 3.76 (m, 9H), 3.75 - 3.67 (m, 2H), 3.14 - 2.90 (m, 2H), 2.81 - 2.77 (m, 1H), 2.39 - 2.28 (m, 1H), 2.23 - 2.09 (m, 2H), 2.07 - 1.91 (m, 4H), 1.65 - 1.49 (m, 2H). |
| 9-7 | 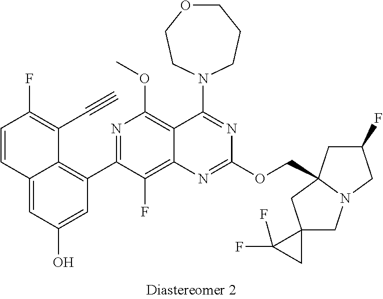 [SMILES: C#Cc1c(F)ccc2cc(O)cc(-c3nc(OC)c4c(N5CCCOCC5)nc(OC[C@@]56C[C@@H](F)CN5CC5(CC5(F)F)C6)nc4c3F)c12] | 5-ethynyl-6- fluoro-4-(8- fluoro-5- methoxy-4-(1,4- oxazepan-4-yl)- 2-(((6′R,7a′R)- 2,2,6′- trifluorodihydro- 1′H,3′H- spiro[cyclopropane- 1,2′- pyrrolizin]- 7a′(5′H)- yl)methoxy)pyrido [4,3- d]pyrimidin-7- yl)naphthalen-2- ol | 682.2 | ¹H NMR (400 MHz, DMSO- d₆) δ ppm = 10.15 (d, J = 1.0 Hz, 1H), 7.97 (dd, J = 9.3, 6.0 Hz, 1H), 7.46 (t, J = 8.9 Hz, 1H), 7.39 (d, J = 2.5 Hz, 1H), 7.23 (d, J = 2.5 Hz, 1H), 5.48 - 5.25 (m, 1H), 4.33 - 4.19 (m, 2H), 4.01 (dd, J = 4.9, 0.9 Hz, 1H), 4.10 (dd, J = 4.9, 0.9 Hz, 1H), 3.89 (s, 3H), 3.87 - 3.80 (m, 5H), 3.76 - 3.66 (m, 2H), 3.29 - 3.25 (br s, 1H), 3.16 (dd, J = 11.6, 5.9 Hz, 1H), 2.89 - 2.69 (m, 2H), 2.39 - 2.27 (m, 1H), 2.19 - 2.10 (m, 1H), 2.07 - 1.86 (m, 4H), 1.68 - 1.44 (m, 2H). |
| 9-8 | 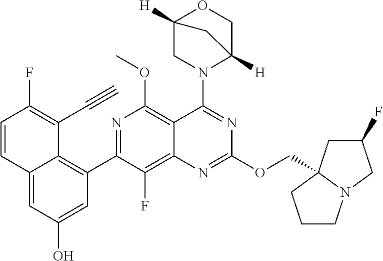 [SMILES: C#Cc1c(F)ccc2cc(O)cc(-c3nc(OC)c4c(N5C[C@H]6C[C@@H]5CO6)nc(OC[C@@]56CCCN5C[C@H](F)C6)nc4c3F)c12] | 4-(4-((1R,4R)-2- oxa-5- azabicyclo[2.2.1] heptan-5-yl)-8- fluoro-2- (((2R,7aS)-2- fluorotetrahydro- 1H-pyrrolizin- 7a(5H)- yl)methoxy)-5- methoxypyrido[4, 3-d]pyrimidin- 7-yl)-5-ethynyl- 6- fluoronaphthalen- 2-ol | 618.2 | ¹H NMR (400 MHz, DMSO- d₆) δ ppm = 10.17-10.14 (m, 1H) 7.97 (dd, J = 9.1, 5.9 Hz, 1H), 7.50 - 7.42 (m, 1H), 7.40 - 7.35 (m, 1H), 7.29 - 7.13 (m, 1H), 5.37 - 5.17 (m, 1H), 4.74 - 4.65 (m, 1H), 4.16 - 4.06 (m, 2H), 4.05 - 3.90 (m, 5H), 3.89 - 3.82 (m, 3H), 3.14 - 2.98 (m, 3H), 2.87 - 2.77 (m, 1H), 2.16 - 1.93 (m, 4H), 1.92 - 1.69 (m, 5H). |
| 9-9 | 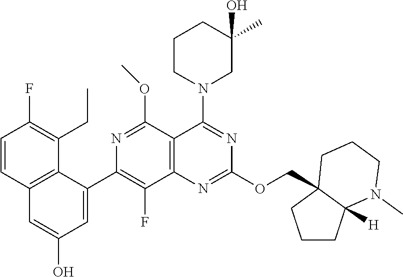 [SMILES: CCc1c(F)ccc2cc(O)cc(-c3nc(OC)c4c(N5CCC[C@@](C)(O)C5)nc(OC[C@]56CCC[C@H]5N(C)CCC6)nc4c3F)c12] | (R)-1-(7-(8- ethyl-7-fluoro-3- hydroxynaphtha- len-1-yl)-8- fluoro-5- methoxy-2- (((4aS,7aR)-1- methyloctahydro- 4aH- cyclopenta[b]pyr- idin-4a- yl)methoxy)pyrido [4,3- d]pyrimidin-4- yl)-3- methylpiperidin- 3-ol | 648.3 | ¹H NMR (400 MHz, DMSO- d₆) δ ppm = 9.94 (s, 1H), 7.78 - 7.74 (m, 1H), 7.39 - 7.30 (m, 2H), 7.08 (dd, J = 10.0, 2.5 Hz, 1H), 4.59 - 4.39 (m, 2H), 4.18 - 4.00 (m, 1H), 3.91 (s, 3H), 3.78 - 3.68 (m, 1H), 3.62 - 3.49 (m, 2H), 3.47 - 3.41 (m, 2H), 2.32 - 2.17 (m, 5H), 2.03 - 1.69 (m, 4H), 1.68 - 1.34 (m, 12H), 1.12 - 1.00 (m, 3H), 0.87 - 0.74 (m, 3H) |
| 9- 10 | 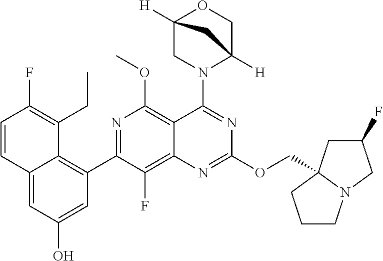 [SMILES: CCc1c(F)ccc2cc(O)cc(-c3nc(OC)c4c(N5C[C@H]6C[C@@H]5CO6)nc(OC[C@@]56CCCN5C[C@H](F)C6)nc4c3F)c12] | 4-(4-((1R,4R)-2- oxa-5- azabicyclo[2.2.1] heptan-5-yl)-8- fluoro-2- (((2R,7aS)-2- fluorotetrahydro- 1H-pyrrolizin- 7a(5H)- yl)methoxy)-5- methoxypyrido[4, 3-d]pyrimidin- 7-yl)-5-ethyl-6- fluoronaphthalen- 2-ol | 622.3 | ¹H NMR (400 MHz, DMSO- d₆) δ ppm = 9.36 - 9.94 (m, 1H), 7.76 (dd, J = 8.6, 6.1 Hz, 1H), 7.46 - 7.29 (m, 2H), 7.17 - 6.90 (m, 1H), 5.47 - 5.13 (m, 1H), 4.71 (br s, 1H), 4.32 - 4.05 (m, 3H), 4.04 - 3.84 (m, 5H), 3.50 - 3.21 (m, 5H), 2.94 - 2.90 (m, 1H), 2.46 - 2.12 (m, 5H), 2.08 - 1.76 (m, 5H), 0.83 - 0.78 (m, 3H). |
| 9- 11 | 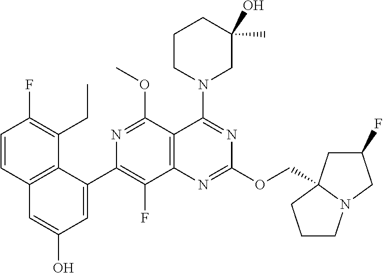 [SMILES: CCc1c(F)ccc2cc(O)cc(-c3nc(OC)c4c(N5CCC[C@@](C)(O)C5)nc(OC[C@@]56CCCN5C[C@H](F)C6)nc4c3F)c12] | (R)-1-(7-(8- ethyl-7-fluoro-3- hydroxynaphthalen- 1-yl)-8- fluoro-2- (((2R,7aS)-2- fluorotetrahydro- 1H-pyrrolizin- 7a(5H)- yl)methoxy)-5- methoxypyrido[4, 3-d]pyrimidin- 4-yl)-3- methylpiperidin- 3-ol | 638.3 | ¹H NMR (400 MHz, DMSO- d₆) δ ppm = 9.92 (s, 1H), 7.82 - 7.67 (m, 1H), 7.42 - 7.27 (m, 2H), 7.08 (dd, J = 10.0, 2.5 Hz, 1H), 5.40 - 5.12 (m, 1H), 4.61 - 4.33 (m, 1H), 4.18 - 4.11 (m, 1H), 4.06 - 4.01 (m, 1H), 3.91 (s, 3H), 3.77 - 3.50 (m, 2H), 3.44 - 3.38 (m, 2H), 3.14 - 2.96 (m, 4H), 2.89 2.79 (m, 1H), 2.31 - 2.21 (m, 1H), 2.17 - 1.94 (m, 3H), 1.90 - 1.72 (m, 3H), 1.64 (br s, 3H), 1.12 - 0.97 (m, 3H), 0.87 - 0.73 (m, 4H). |
| 9- 12 | 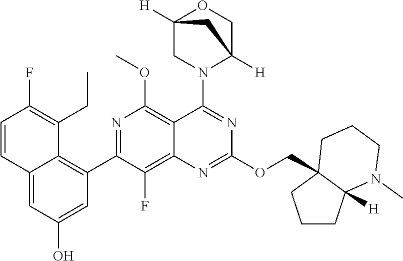 [SMILES: CCc1c(F)ccc2cc(O)cc(-c3nc(OC)c4c(N5C[C@H]6C[C@@H]5CO6)nc(OC[C@]56CCC[C@H]5N(C)CCC6)nc4c3F)c12] | 4-(4-((1R,4R)-2- oxa-5- azabicyclo[2.2.1] heptan-5-yl)-8- fluoro-5- methoxy-2- (((4aS,7aR)-1- methyloctahydro- 4aH- cyclopenta[b]pyr- idin-4a- yl)methoxy)pyrido [4,3- d]pyrimidin-7- yl)-5-ethyl-6- fluoronaphthalen- 2-ol | 632.3 | ¹H NMR (400 MHz, DMSO- d₆) δ ppm = 9.92 - 9.91 (m, 1H), 7.76 (dd, J = 8.8, 6.0 Hz, 1H), 7.40 - 7.29 (m, 2H), 7.16 - 6.93 (m, 1H), 4.70 (br s, 1H), 4.53-4.45 (m, 1H), 4.11 - 3.95 (m, 5H), 3.88 - 3.87 (m, 3H), 3.50 - 3.20 (m, 2H), 2.71 - 2.69 (m, 1H), 2.24 - 2.19 (m, 4H), 1.91 - 133 (m, 14H), 0.83 - 0.78 (m, 3H). |
| 9- 13 | 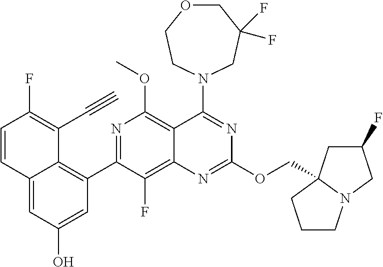 [SMILES: C#Cc1c(F)ccc2cc(O)cc(-c3nc(OC)c4c(N5CCOCC(F)(F)C5)nc(OC[C@@]56CCCN5C[C@H](F)C6)nc4c3F)c12] | 4-(4-(6,6- difluoro-1,4- oxazepan-4-yl)- 8-fluoro-2- (((2R,7aS)-2- fluorotetrahydro- 1H-pyrrolizin- 7a(5H)- yl)methoxy)-5- methoxypyrido[4, 3-d]pyrimidin- 7-yl)-5-ethynyl- 6- fluoronaphthalen- 2-o1 | 656.2 | ¹H NMR (400 MHz, DMSO- d₆) δ ppm = 10.18 (s, 1H), 7.98 (dd, J = 9.3, 6.0 Hz, 1H), 7.47 (t, J = 8.9 Hz, 1H), 7.40 (d, J = 2.5 Hz, 1H), 7.25 (d, J = 2.0 Hz, 1H), 5.46 - 5.14 (m, 1H), 4.74 - 4.58 (m, 1H), 4.36 - 4.24 (m, 1H), 4.21 - 3.92 (m, 7H), 3.93 - 3.91 (m, 3H), 3.85 - 3.80 (m, 2H), 3.40 - 3.30 (m, 1H), 3.19 - 3.15 (m, 2H), 2.89 (br s, 1H), 2.22 - 1.97 (m, 3H), 1.95 - 1.69 (m, 3H). |
| 9- 14 | 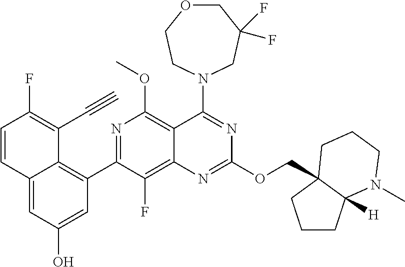 [SMILES: C#Cc1c(F)ccc2cc(O)cc(-c3nc(OC)c4c(N5CCOCC(F)(F)C5)nc(OC[C@]56CCC[C@H]5N(C)CCC6)nc4c3F)c12] | 4-(4-(6,6- difluoro-1,4- oxazepan-4-yl)- 8-fluoro-5- methoxy-2- (((4aS,7aR)-1- methyloctahydro- 4aH- cyclopenta[b]pyr- idin-4a- yl)methoxy)pyri- do[4,3- d]pyrimidin-7- yl)-5-ethynyl-6- fluoronaphthalen- 2-ol | 666.3 | ¹H NMR (400 MHz, DMSO- d₆) δ ppm = 10.18 (s, 1H), 7.98 (dd, J = 9.3, 6.0 Hz, 1H), 7.47 (t, J = 9.0 Hz, 1H), 7.40 (d, J = 2.5 Hz, 1H), 7.25 (d, J = 2.5 Hz, 1H), 4.74 - 4.60 (m, 1H), 4.54 - 4.43 (m, 1H), 4.35 - 4.14 (m, 2H), 4.12 - 3.96 (m, 4H), 3.94 - 3.90 (m, 3H), 3.85 - 3.81 (m, 3H), 2.79 - 2.75 (m, 1H), 2.27 -2.21 (m, 4H), 1.90 - 1.81 (m, 1H), 1.78 - 1.70 (m, 1H), 1.68 - 1.52 (m, 8H), 1.48 - 1.33 (m, 1H). |
| 9- 15 | 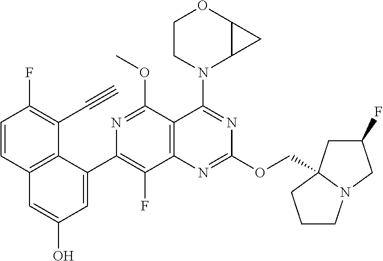 [SMILES: C#Cc1c(F)ccc2cc(O)cc(-c3nc(OC)c4c(N5CCOC6CC65)nc(OC[C@@]56CCCN5C[C@H](F)C6)nc4c3F)c12] | 4-(4-(2-oxa-5- azabicyclo[4.1.0] heptan-5-yl)-8- fluoro-2- (((2R,7aS)-2- fluorotetrahydro- 1H-pyrrolizin- 7a(5H)- yl)methoxy)-5- methoxypyrido[4, 3-d]pyrimidin- 7-yl)-5-ethynyl- 6- fluoronaphthalen- 2-ol | 618.3 | ¹H NMR (400 MHz, DMSO- d₆) δ ppm = 10.15 (d, J = 3.8 Hz, 1H), 8.01 - 7.94 (m, 1H), 7.46 (td, J = 8.9, 2.6 Hz, 1H), 7.39 (d, J = 2.5 Hz, 1H), 7.24 (dd, J = 7.4, 1.4 Hz, 1H), 5.40 - 5.20 (m, 1H), 4.22 - 4.02 (m, 3H), 3.91 (d, J = 5.5 Hz, 3H), 3.83 - 3.64 (m, 3H), 3.58 - 3.40 (m, 2H), 3.19 - 3.02 (m, 4H), 2.91 - 2.81 (m, 1H), 2.18 - 1.99 (m, 3H), 1.90 - 1.75 (m, 3H), 0.97 - 0.70 (m, 2H) (Diastereomeric mixture) |
| 9- 16 | 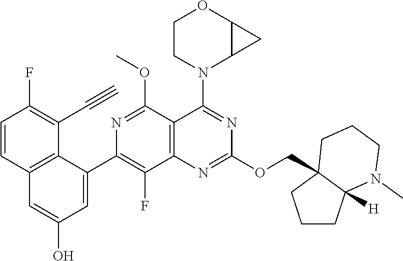 [SMILES: C#Cc1c(F)ccc2cc(O)cc(-c3nc(OC)c4c(N5CCOC6CC65)nc(OC[C@]56CCC[C@H]5N(C)CCC6)nc4c3F)c12] | 4-(4-(2-oxa-5- azabicyclo[4.1.0] heptan-5-yl)-8- fluoro-5- methoxy-2- (((4aS,7aR)-1- methyloctahydro- 4aH- cyclopenta[b]pyr- idin-4a- yl)methoxy)pyri- do[4,3- d]pyrimidin-7- yl)-5-ethynyl-6- fluoronaphthalen- 2-ol | 628.3 | ¹H NMR (400 MHz, DMSO- d₆) δ ppm = 10.15 (br s, 1H), 8.02 - 7.95 (m, 1H), 7.48 - 7.44 (m, 1H), 7.39 (d, J = 2.3 Hz, 1H), 7.27 - 7.21 (m, 1H), 4.51 - 4.41 (m, 1H), 4.21 - 4.12 (m, 1H), 4.08 - 4.04 (m, 1H), 3.94 - 3.87 (m, 3H), 3.82 - 3.63 (m, 4H), 3.58 - 3.45 (m, 2H), 3.18 - 3.11 (m, 1H), 2.72 (br s, 1H), 2.31 - 2.18 (m, 4H), 1.89 - 1.79 (m, 1H), 1.77 - 1.49 (m, 8H), 1.44 - 1.36 (m, 1H), 0.96 - 0.68 (m, 2H) (Diastereomeric mixture) |
| 9- 17 | 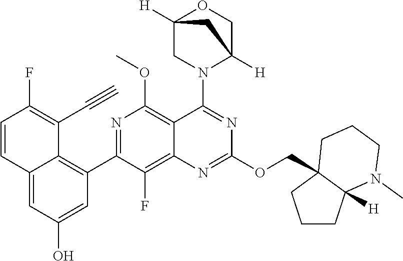 [SMILES: C#Cc1c(F)ccc2cc(O)cc(-c3nc(OC)c4c(N5C[C@H]6C[C@@H]5CO6)nc(OC[C@]56CCC[C@H]5N(C)CCC6)nc4c3F)c12] | 4-(4-((1R,4R)-2- oxa-5- azabicyclo[2.2.1] heptan-5-yl)-8- fluoro-5- methoxy-2- (((4aS,7aR)-1- methyloctahydro- 4aH- cyclopenta[b]pyr- idin-4a- yl)methoxy)pyri- do[4,3- d]pyrimidin-7- yl)-5-ethynyl-6- fluoronaphthalen- 2-ol | 628.2 | ¹H NMR (400 MHz, DMSO- d₆) δ ppm = 10.21 (br s, 1H), 7.97 (dd, J = 9.1, 5.9 Hz, 1H), 7.50 - 7.42 (m, 1H), 7.41 - 7.37 (m, 1H), 7.35 -7.16 (m, 1H), 4.59 -4.38 (m, 3H), 4.20 - 4.04 (m, 2H), 3.96 - 3.81 (m, 4H), 3.51 - 3.45 (m, 3H), 3.11 - 3.04 (m, 1H), 2.67 - 2.63 (m, 1H), 2.27 - 2.11 (m, 4H), 2.05 - 1.77 (m, 2H), 1.75 - 1.47 (m, 10H). |
| 9- 18 | 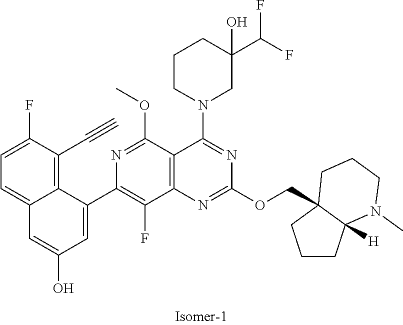 [SMILES: C#Cc1c(F)ccc2cc(O)cc(-c3nc(OC)c4c(N5CCCC(O)(C(F)F)C5)nc(OC[C@]56CCC[C@H]5N(C)CCC6)nc4c3F)c12] | 3- (difluoromethyl)- 1-(7-(8-ethynyl- 7-fluoro-3- hydroxynaphtha- len-1-yl)-8- fluoro-5- methoxy-2- (((4aS,7aR)-1- methyloctahydro- 4aH- cyclopenta[b]pyr- idin-4a- yl)methoxy)pyri- do[4,3- d]pyrimidin-4- yl)piperidin-3-ol | 680.3 | ¹H NMR (400 MHz, DMSO- d₆) δ ppm = 10.28 - 10.10 (m, 1H), 7.97 (dd, J = 9.3, 5.8 Hz, 1H), 7.55 - 7.42 (m, 1H), 7.40 (d, J = 2.5 Hz, 1H), 7.31 - 7.13 (m, 1H), 5.83 (t, J = 56.0 Hz, 1H), 5.59 - 5.28 (m, 1H), 4.56 - 4.33 (m, 2H), 4.27 - 4.08 (m, 1H), 4.03 - 3.96 (m, 1H), 3.94 - 3.84 (m, 3H), 3.77 - 3.49 (m, 2H), 3.18 - 3.01 (m, 1H), 2.70 - 2.66 (m, 1H), 2.30 - 2.22 (m, 1H), 2.22 (br s, 3H), 2.10 - 1.97 (m, 1H), 1.88 - 1.68 (m, 4H), 1.67 - 1.48 (m, 9H), 1.47 - 1.35 (m, 1H). |
| 9- 19 | 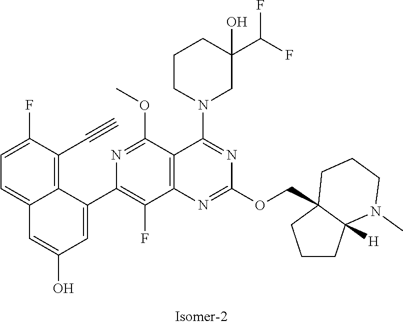 [SMILES: C#Cc1c(F)ccc2cc(O)cc(-c3nc(OC)c4c(N5CCCC(O)(C(F)F)C5)nc(OC[C@]56CCC[C@H]5N(C)CCC6)nc4c3F)c12] | 3- (difluoromethyl)- 1-(7-(8-ethynyl- 7-fluoro-3- hydroxynaphtha- len-1-yl)-8- fluoro-5- methoxy-2- (((4aS,7aR)-1- methyloctahydro- 4aH- cyclopenta[b]pyr- idin-4a- yl)methoxy)pyri- do[4,3- d]pyrimidin-4- yl)piperidin-3-ol | 680.3 | ¹H NMR (400 MHz, DMSO- d₆) δ ppm = 10.26 - 10.08 (m, 1H), 7.97 (dd, J = 9.1, 5.9 Hz, 1H), 7.52 - 7.42 (m, 1H), 7.39 (d, J = 2.5 Hz, 1H), 7.30 - 7.08 (m, 1H), 5.85 (t, J = 56.0 Hz, 1H), 5.54 - 5.24 (m, 1H), 4.56 - 4.44 (m, 2H), 4.11 (d, J = 10.8 Hz, 1H), 3.98 - 3.85 (m, 4H), 3.79 - 3.48 (m, 2H), 3.10 -3.02 (m, 1H), 2.67 (br s, 1H), 2.21 - 2.19 (m, 1H), 2.19 (s, 3H), 2.14 - 1.95 (m, 1H), 1.87 - 1.67 (m, 4H), 1.67 - 1.46 (m, 9H), 1.45 - 1.30 (m, 1H). |
| 9- 20 | 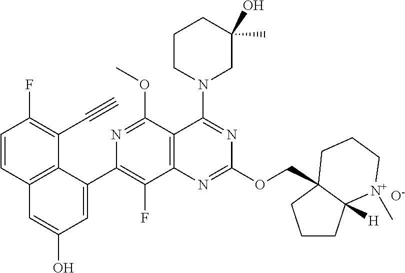 | (4aS,7aR)-4a- (((7-(8-ethynyl- 7-fluoro-3- hydroxynaphtha- len-1-yl)-8- fluoro-4-((R)-3- hydroxy-3- methylpiperidin- 1-yl)-5- methoxypyrido[4, 3-d]pyrimidin- 2- yl)oxy)methyl)- 1- methyloctahydro- 1H- cyclopenta[b]pyr- | 660.2 | ¹H NMR (400 MHz, DMSO- d₆) δ ppm = 10.52 - 10.11 (brs, 1H), 7.89 (dd, J = 9.3, 6.0 Hz, 1H), 7.60 (br s, 1H), 7.38 (t, J = 9.1 Hz, 1H), 7.32 - 7.25 (m, 1H), 5.82 - 5.14 (m, 1H), 4.42 (br s, 2H), 4.02 - 3.82 (m, 5H), 3.55 - 3.41 (m, 2H), 3.16 - 2.95 (m, 4H), 2.92 - 2.71 (m, 1H), 2.45 - 2.40 (m, 2H), 2.16 - 1.91 (m, 3H), 1.89 - 1.31 (m, 11H), 1.17 - 0.95 (m, 3H). |
| | | idine 1-oxide | | |
| 9- 21 | 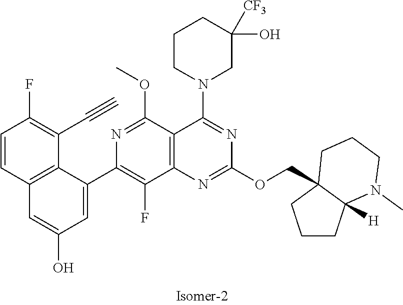 [SMILES: C#Cc1c(F)ccc2cc(O)cc(-c3nc(OC)c4c(N5CCCC(O)(C(F)(F)F)C5)nc(OC[C@]56CCC[C@H]5N(C)CCC6)nc4c3F)c12] | 1-(7-(8-ethynyl- 7-fluoro-3- hydroxynaphtha- len-1-yl)-8- fluoro-5- methoxy-2- (((4aS,7aR)-1- methyloctahydro- 4aH- cyclopenta[b]pyr- idin-4a- yl)methoxy)pyri- do[4,3- d]pyrimidin-4- yl)-3- (trifluoromethyl) piperidin-3-ol | 698.2 | ¹H NMR (400 MHz, DMSO- d₆) δ ppm = 10.19 (br s, 1H), 8.04 - 7.84 (m, 1H), 7.53 - 7.42 (m, 1H), 7.42 - 7.37 (m, 1H), 7.33 - 7.14 (m, 1H), 6.39 - 5.92 (m, 1H), 4.55 - 4.32 (m, 2H), 4.25 - 4.06 (m, 1H), 3.95 - 3.80 (m, 5H), 3.67 - 3.55 (m, 1H), 3.19 - 3.13 (m, 1H), 2.74 (br s, 1H), 2.26 - 2.23 (m, 1H), 2.24 (s, 3H), 2.13 - 2.05 (m, 1H), 1.97 - 1.80 (m, 4H), 1.78 - 1.51 (m, 9H), 1.48 - 1.35 (m, 1H). |
| 9- 22 | 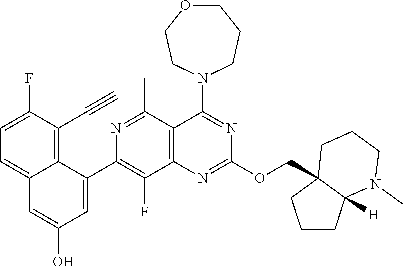 [SMILES: C#Cc1c(F)ccc2cc(O)cc(-c3nc(C)c4c(N5CCCOCC5)nc(OC[C@]56CCC[C@H]5N(C)CCC6)nc4c3F)c12] | 5-ethynyl-6- fluoro-4-(8- fluoro-5-methyl- 2-(((4aS,7aR)-1- methyloctahydro- 4aH- cyclopenta[b]pyr- idin-4a- yl)methoxy)-4- (1,4-oxazepan-4- yl)pyrido[4,3- d]pyrimidin-7- yl)naphthalen-2- ol | 614.3 | ¹H NMR (400 MHz, DMSO- d₆) δ ppm = 10.5 - 9.8 (brs, 1H), 7.97 (dd, J = 9.1, 5.9 Hz, 1H), 7.46 (t, J = 9.0 Hz, 1H), 7.38 (d, J = 2.5 Hz, 1H), 7.23 (s, 1H), 4.45 (dd, J = 10.9, 5.4 Hz, 1H), 4.14 (d, J = 10.8 Hz, 1H), 4.04 - 3.58 (m, 5H), 2.68 - 2.55 (m, 4H), 2.46 (br d, J = 2.8 Hz, 1H), 2.30 - 2.22 (m, 1H), 2.19 (s, 3H), 2.1 - 2.0 (m, 2H), 1.91 (s, 3H), 1.87 - 1.70 (m, 3H), 1.65 - 1.36 (m, 9H). |
| 9- 23 | 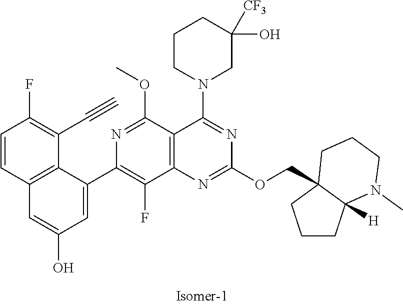 [SMILES: C#Cc1c(F)ccc2cc(O)cc(-c3nc(OC)c4c(N5CCCC(O)(C(F)(F)F)C5)nc(OC[C@]56CCC[C@H]5N(C)CCC6)nc4c3F)c12] | 1-(7-(8-ethynyl- 7-fluoro-3- hydroxynaphtha- len-1-yl)-8- fluoro-5- methoxy-2- (((4aS,7aR)-1- methyloctahydro- 4aH- cyclopenta[b]pyr- idin-4a- yl)methoxy)pyri- do[4,3- d]pyrimidin-4- yl)-3- (trifluoromethyl) piperidin-3-ol | 698.2 | ¹H NMR (400 MHz, DMSO- d₆) δ ppm = 10.19 (br s, 1H), 8.04 - 7.92 (m, 1H), 7.53 - 7.37 (m, 2H), 7.34 - 7.14 (m, 1H), 6.34 (d, J = 1.0 Hz, 1H), 4.50 (d, J = 10.8 Hz, 2H), 4.13 (d, J = 10.8 Hz, 1H), 3.96 - 3.76 (m, 5H), 3.67 - 3.59 (m, 1H), 3.19 - 3.11 (m, 1H), 2.72 (br d, J = 5.0 Hz, 1H), 2.31 - 2.25 (m, 1H), 2.22 (s, 3H), 2.16 - 2.02 (m, 1H), 1.95 - 1.80 (m, 4H), 1.75 - 1.48 (m, 9H), 1.45 - 1.33 (m, 1H). |
| 9- 24 | 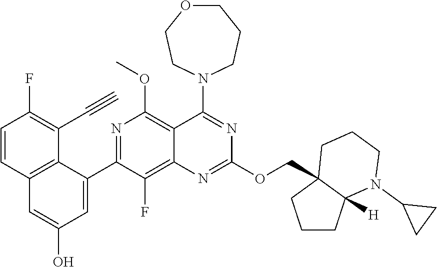 [SMILES: C#Cc1c(F)ccc2cc(O)cc(-c3nc(OC)c4c(N5CCCOCC5)nc(OC[C@]56CCC[C@H]5N(C5CC5)CCC6)nc4c3F)c12] | 4-(2-(((4aS,7aR)- 1- cyclopropylocta- hydro-4aH- cyclopenta[b]pyr- idin-4a- yl)methoxy)-8- fluoro-5- methoxy-4-(1,4- oxazepan-4- yl)pyrido[4,3- d]pyrimidin-7- yl)-5-ethynyl-6- fluoronaphthalen- 2-ol | 655.8 | ¹H NMR (400 MHz, DMSO- d₆) δ ppm = 10.21 (br s, 1H), 7.97 (dd, J = 9.1, 5.9 Hz, 1H), 7.46 (t, J = 9.0 Hz, 1H), 7.38 (d, J = 2.5 Hz, 1H), 7.23 (s, 1H), 4.45 (dd, J = 10.6, 4.1 Hz, 1H), 4.14 (t, J = 9.9 Hz, 1H), 4.00 (d, J = 5.5 Hz, 1H), 3.92 - 3.77 (m, 9H), 3.76 - 3.69 (m, 2H), 3.09 - 2.94 (m, 1H), 2.64- 2.53 (m, 2H), 2.08 - 1.91 (m, 3H), 1.73 (dt, J = 6.4, 3.4 Hz, 2H), 1.67 - 1.27 (m, 8H), 0.52 - 0.04 (m, 4H). |
| 9- 25 | 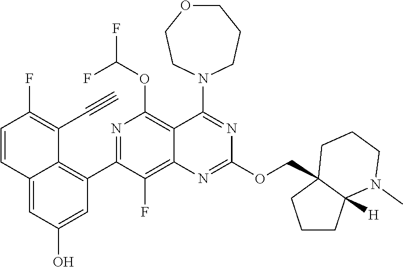 [SMILES: C#Cc1c(F)ccc2cc(O)cc(-c3nc(OC(F)F)c4c(N5CCCOCC5)nc(OC[C@]56CCC[C@H]5N(C)CCC6)nc4c3F)c12] | 4-(5- (difluoromethoxy)- 8-fluoro-2- (((4aS,7aR)-1- methyloctahydro- 4aH- cyclopenta[b]pyr- idin-4a- yl)methoxy)-4- (1,4-oxazepan-4- yl)pyrido[4,3- d]pyrimidin-7- yl)-5-ethynyl-6- fluoronaphthalen- 2-ol | 666.2 | ¹H NMR (400 MHz, DMSO- d₆) δ ppm = 10.25 (s, 1H), 7.98 (dd, J = 9.1, 5.9 Hz, 1H), 7.69 (t, J = 71.3 Hz, 1H), 7.48 (t, J = 9.1 Hz, 1H), 7.41 (d, J = 2.8 Hz, 1H), 7.26 (d, J = 2.5 Hz, 1H), 4.46 (d, J = 11.0 Hz, 1H), 4.17 (dd, J = 10.8, 5.0 Hz, 1H), 4.11 - 4.09 (m, 1H), 3.98 - 3.82 (m, 6H), 3.76 - 3.64 (m, 2H), 2.76 - 2.65 (m, 1H), 2.48 - 2.42 (m, 1H), 2.27 - 2.21 (m, 1H), 2.17 (s, 3H), 2.09 - 2.00 (m, 2H), 1.88 - 1.68 (m, 2H), 1.65 - 1.45 (m, 7H), 1.45 - 1.35 (m, 1H). |
| 9- 26 | 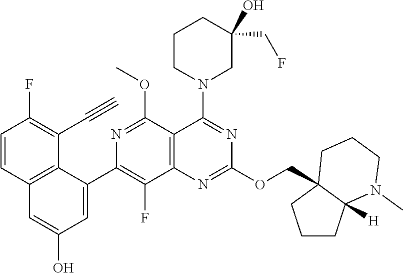 [SMILES: C#Cc1c(F)ccc2cc(O)cc(-c3nc(OC)c4c(N5CCC[C@](O)(CF)C5)nc(OC[C@]56CCC[C@H]5N(C)CCC6)nc4c3F)c12] | (R)-1-(7-(8- ethynyl-7-fluoro- 3- hydroxynaphtha- len-1-yl)-8- fluoro-5- methoxy-2- (((4aS,7aR)-1- methyloctahydro- 4aH- cyclopenta[b]pyr- idin-4a- yl)methoxy)pyri- do[4,3- d]pyrimidin-4- yl)-3- (fluoromethyl)pi- | 662.3 | ¹H NMR (400 MHz, DMSO- d₆) δ ppm = 10.26 - 10.08 (m, 1H), 8.00 - 7.94 (m, 1H), 7.51 - 7.43 (m, 1H), 7.39 (d, J = 2.5 Hz, 1H), 7.29 - 7.21 (m, 1H), 5.06 - 4.98 (m, 1H), 4.51 - 4.08 (m, 5H), 4.05 (s, 1H), 3.98 - 3.82 (m, 3H), 3.71 - 3.56 (m, 2H), 3.19 - 3.07 (m, 2H), 2.71 (br d, J = 2.4 Hz, 1H), 2.32 - 2.19 (m, 4H), 2.12 - 1.98 (m, 1H), 1.90 - 1.80 (m, 1H), 1.77 - 1.48 (m, 11H), 1.46 - 1.36 (m, 1H). |
| | | peridin-3-ol | | |
| 9- 27 | 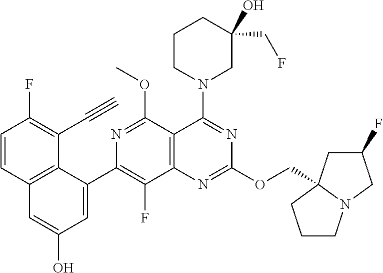 [SMILES: C#Cc1c(F)ccc2cc(O)cc(-c3nc(OC)c4c(N5CCC[C@](O)(CF)C5)nc(OC[C@@]56CCCN5C[C@H](F)C6)nc4c3F)c12] | (R)-1-(7-(8- ethynyl-7-fluoro- 3- hydroxynaphtha- len-1-yl)-8- fluoro-2- (((2R,7aS)-2- fluorotetrahydro- 1H-pyrrolizin- 7a(5H)- yl)methoxy)-5- methoxypyrido[4, 3-d]pyrimidin- 4-yl)-3- (fluoromethyl)pi- peridin-3-ol | 652.2 | ¹H NMR (400 MHz, DMSO- d₆) δ ppm = 10.18 - 10.13 (m, 1H), 7.97 (dd, J = 9.1, 5.9 Hz, 1H), 7.50 - 7.43 (m, 1H), 7.39 (d, J = 2.4 Hz, 1H), 7.29 - 7.20 (m, 1H), 5.41 - 5.22 (m, 1H), 5.04 - 4.98 (m, 1H), 4.45 - 4.02 (m, 5H), 3.96 - 3.82 (m, 4H), 3.71 - 3.56 (m, 2H), 3.20 - 3.06 (m, 4H), 2.95 - 2.83 (m, 1H), 2.22 - 1.97 (m, 4H), 1.93 - 1.55 (m, 6H). |
| 9- 28 | 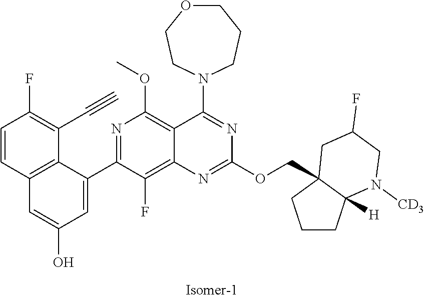 [SMILES: [2H]C([2H])([2H])N1CC(F)C[C@@]2(COc3nc(N4CCCOCC4)c4c(OC)nc(-c5cc(O)cc6ccc(F)c(C#C)c56)c(F)c4n3)CCC[C@@H]12] | 5-ethynyl-6- fluoro-4-(8- fluoro-2- (((4aS,7aR)-3- fluoro-1- (methyl- d3)octahydro- 4aH- cyclopenta[b]pyr- idin-4a- yl)methoxy)-5- methoxy-4-(1,4- oxazepan-4- yl)pyrido[4,3- d]pyrimidin-7- yl)naphthalen-2- ol | 651.2 | ¹H NMR (400 MHz, DMSO- d₆) δ ppm = 10.14 (s, 1H), 7.97 (br d, J = 3.3 Hz, 1H), 7.46 (t, J = 9.1 Hz, 1H), 7.39 (d, J = 2.5 Hz, 1H), 7.23 (d, J = 2.5 Hz, 1H), 4.92 - 4.70 (m, 1H), 4.42 - 4.31 (m, 1H), 4.23 (br d, J = 10.8 Hz, 1H), 4.02 (d, J = 3.8 Hz, 1H), 3.89 (s, 3H), 3.84 (s, 5H), 3.79 - 3.67 (m, 3H), 2.86 - 2.69 (m, 2H), 2.45 - 2.37 (m, 1H), 2.08 - 2.00 (m, 2H), 1.91 - 1.53 (m, 8H). |
| 9- 29 | 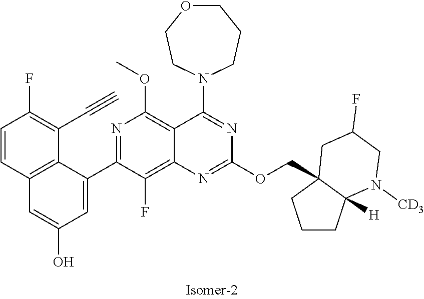 [SMILES: [2H]C([2H])([2H])N1CC(F)C[C@@]2(COc3nc(N4CCCOCC4)c4c(OC)nc(-c5cc(O)cc6ccc(F)c(C#C)c56)c(F)c4n3)CCC[C@@H]12] | 5-ethynyl-6- fluoro-4-(8- fluoro-2- (((4aS,7aR)-3- fluoro-1- (methyl- d3)octahydro- 4aH- cyclopenta[b]pyr- idin-4a- yl)methoxy)-5- methoxy-4-(1,4- oxazepan-4- yl)pyrido[4,3- d]pyrimidin-7- yl)naphthalen-2- ol | 651.2 | ¹H NMR (400 MHz, DMSO- d₆) δ ppm = 10.16 (s, 1H), 7.97 (dd, J = 9.3, 6.0 Hz, 1H), 7.46 (t, J = 9.0 Hz, 1H), 7.39 (d, J = 2.5 Hz, 1H), 7.23 (d, J = 2.5 Hz, 1H), 4.94 - 4.67 (m, 1H), 4.46 - 4.29 (m, 1H), 4.11 (dd, J = 10.6, 2.6 Hz, 1H), 4.01 (s, 1H), 3.93 - 3.81 (m, 10H), 3.77 - 3.69 (m, 2H), 2.76 - 2.69 (m, 2H), 2.10 - 1.96 (m, 2H), 1.89 - 1.43 (m, 8H). |
| 9- 30 | 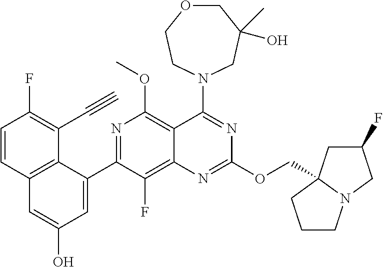 [SMILES: C#Cc1c(F)ccc2cc(O)cc(-c3nc(OC)c4c(N5CCOCC(C)(O)C5)nc(OC[C@@]56CCCN5C[C@H](F)C6)nc4c3F)c12] | 4-(7-(8-ethynyl- 7-fluoro-3- hydroxynaphtha- len-1-yl)-8- fluoro-2- (((2R,7aS)-2- fluorotetrahydro- 1H-pyrrolizin- 7a(5H)- yl)methoxy)-5- methoxypyrido[4, 3-d]pyrimidin- 4-yl)-6-methyl- 1,4-oxazepan-6- ol | 650.3 | ¹H NMR (400 MHz, DMSO- d₆) δ ppm = 10.37 (brs, 1H), 7.97 (dd, J = 9.1, 5.9 Hz, 1H), 7.48 (t, J = 9.1 Hz, 1H), 7.39 (d, J = 2.5 Hz, 1H), 7.28 - 7.20 (m, 1H), 5.44 - 4.93 (m, 2H), 4.17 - 3.87 (m, 9H), 3.75 - 3.58 (m, 3H), 3.50 - 3.40 (m, 2H), 3.13 - 2.98 (m, 3H), 2.87 - 2.78 (m, 1H), 2.20 - 1.95 (m, 3H), 1.89 - 1.72 (m, 3H), 1.15 - 0.93 (m, 3H). |
| 9- 31 | 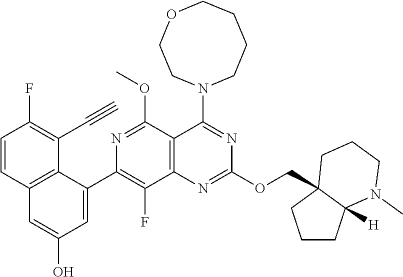 [SMILES: C#Cc1c(F)ccc2cc(O)cc(-c3nc(OC)c4c(N5CCCCOCC5)nc(OC[C@]56CCC[C@H]5N(C)CCC6)nc4c3F)c12] | 5-ethynyl-6- fluoro-4-(8- fluoro-5- methoxy-2- (((4aS,7aR)-1- methyloctahydro- 4aH- cyclopenta[b]pyr- idin-4a- yl)methoxy)-4- (1,4-oxazocan-4- yl)pyrido[4,3- d]pyrimidin-7- yl)naphthalen-2- ol | 645.0 | ¹H NMR (400 MHz, DMSO- d₆) δ ppm = 10.23 - 10.10 (m, 1H), 7.97 (dd, J = 9.3, 5.8 Hz, 1H), 7.46 (t, J = 9.0 Hz, 1H), 7.39 (d, J = 2.5 Hz, 1H), 7.24 (d, J = 2.5 Hz, 1H), 4.42 (dd, J = 10.6, 5.9 Hz, 1H), 4.31 - 4.23 (m, 1H), 4.15 (dd, J = 10.6, 6.4 Hz, 1H), 4.03 (s, 1H), 4.00 - 3.94 (m, 2H), 3.92 - 3.86 (m, 1H), 3.88 (s, 3H), 3.78 - 3.66 (m, 2H), 3.61 - 3.52 (m, 2H), 2.66 - 2.61 (m, 1H), 2.48 - 2.42 (m, 1H), 2.20 - 2.16 (m, 1H), 2.18 (s, 3H), 2.02 - 1.91 (m, 1H), 1.88 - 1.68 (m, 4H), 1.66 - 1.48 (m, |
| | | | | 8H), 1.44 - 1.34 (m, 1H). |
| 9- 32 | 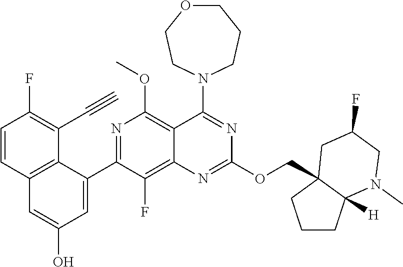 [SMILES: C#Cc1c(F)ccc2cc(O)cc(-c3nc(OC)c4c(N5CCCOCC5)nc(OC[C@]56CCC[C@H]5N(C)C[C@H](F)C6)nc4c3F)c12] | 5-ethynyl-6- fluoro-4-(8- fluoro-2- (((3R,4aS,7aR)- 3-fluoro-1- methyloctahydro- 4aH- cyclopenta[b]pyr- idin-4a- yl)methoxy)-5- methoxy-4-(1,4- oxazepan-4- yl)pyrido[4,3- d]pyrimidin-7- yl)naphthalen-2- ol | 649.0 | ¹H NMR (400 MHz, DMSO- d₆) δ ppm = 10.12 (s, 1H), 7.97 (dd, J = 9.4, 5.9 Hz, 1H), 7.46 (t, J = 9.1 Hz, 1H), 7.38 (d, J = 2.8 Hz, 1H), 7.23 (d, J = 2.5 Hz, 1H), 4.92 - 4.68 (m, 1H), 4.43 - 4.33 (m, 1H), 4.27 - 4.20 (m, 1H), 4.06 - 3.97 (m, 1H), 3.92 - 3.81 (m, 10H), 3.81 -3.69 (m, 2H), 2.60 - 2.72 (m, 2H), 2.26 - 2.19 (m, 3H), 2.10 - 2.00 (m, 2H), 1.91 - 1.54 (m, 8H). |
| 9- 33 | 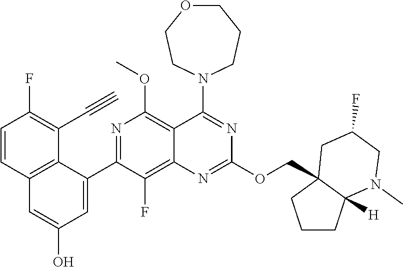 [SMILES: C#Cc1c(F)ccc2cc(O)cc(-c3nc(OC)c4c(N5CCCOCC5)nc(OC[C@]56CCC[C@H]5N(C)C[C@@H](F)C6)nc4c3F)c12] | 5-ethynyl-6- fluoro-4-(8- fluoro-2- (((3S,4aS,7aR)- 3-fluoro-1- methyloctahydro- 4aH- cyclopenta[b]pyr- idin-4a- yl)methoxy)-5- methoxy-4-(1,4- oxazepan-4- yl)pyrido[4,3- d]pyrimidin-7- yl)naphthalen-2- ol | 649.0 | ¹H NMR (400 MHz, DMSO- d₆) δ ppm = 7.96 (dd, J = 9.3, 5.8 Hz, 1H), 7.46 (t, J = 9.1 Hz, 1H), 7.38 (d, J = 2.8 Hz, 1H), 7.23 (d, J = 2.8 Hz, 1H), 4.92 - 4.66 (m, 1H), 4.38 (t, J = 10.5 Hz, 1H), 4.17 - 4.08 (m, 1H), 4.01 (dd, J = 2.0, 1.0 Hz, 1H), 3.92 - 3.82 (m, 10H), 3.77 - 3.70 (m, 2H), 2.72 - 2.57 (m, 2H), 2.23 (d, J = 1.0 Hz, 3H), 2.13 - 1.97 (m, 3H), 1.92 - 1.48 (m, 8H). |
| 9- 34 | 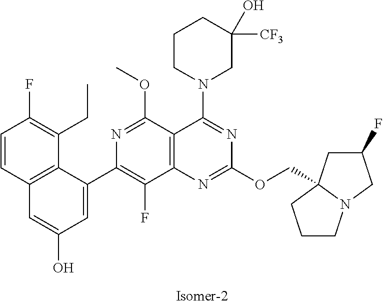 [SMILES: CCc1c(F)ccc2cc(O)cc(-c3nc(OC)c4c(N5CCCC(O)(C(F)(F)F)C5)nc(OC[C@@]56CCCN5C[C@H](F)C6)nc4c3F)c12] | 1-(7-(8-ethyl-7- fluoro-3- hydroxynaphtha- len-1-yl)-8- fluoro-2- (((2R,7aS)-2- fluorotetrahydro- 1H-pyrrolizin- 7a(5H)- yl)methoxy)-5- Isomer-2 methoxypyrido[4, 3-d]pyrimidin- 4-yl)-3- (trifluoromethyl) piperidin-3-ol | 692.3 | ¹H NMR (400 MHz, DMSO- d₆) δ ppm = 10.00 - 9.87 (m, 1H), 7.82 - 7.69 (m, 1H), 7.40 - 7.27 (m, 2H), 7.16 - 6.99 (m, 1H), 6.15 - 5.93 (m, 1H), 5.42 - 5.14 (m, 1H), 4.42 - 4.28 (m, 1H), 4.21 - 4.11 (m, 1H), 4.08 - 4.00 (m, 1H), 3.96 - 3.83 (m, 4H), 3.67 - 3.54 (m, 1H), 3.23 - 3.14 (m, 1H), 3.10 (br dd, J = 5.5, 3.0 Hz, 2H), 3.04 - 3.01 (m, 1H), 2.92 - 2.79 (m, 1H), 2.32 - 2.19 (m, 2H), 2.18 - 1.97 (m, 4H), 1.93 - 1.63 (m, 6H), 0.82 - 0.76 (m, 3H). |
| 9- 35 | 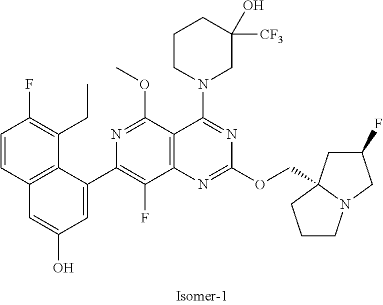 [SMILES: CCc1c(F)ccc2cc(O)cc(-c3nc(OC)c4c(N5CCCC(O)(C(F)(F)F)C5)nc(OC[C@@]56CCCN5C[C@H](F)C6)nc4c3F)c12] | 1-(7-(8-ethyl-7- fluoro-3- hydroxynaphtha- len-1-yl)-8- fluoro-2- (((2R,7aS)-2- fluorotetrahydro- 1H-pyrrolizin- 7a(5H)- yl)methoxy)-5- methoxypyrido[4, 3-d]pyrimidin- 4-yl)-3- (trifluoromethyl) piperidin-3-ol | 692.3 | ¹H NMR (400 MHz, DMSO- d₆) δ ppm = 10.01 - 9.84 (m, 1H), 7.76 (ddd, J = 8.9, 6.1, 2.8 Hz, 1H), 7.42 - 7.27 (m, 2H), 7.15 - 7.01 (m, 1H), 6.13 - 5.98 (m, 1H), 5.40 - 5.15 (m, 1H), 4.43 - 4.23 (m, 1H), 4.19 - 4.02 (m, 2H), 4.00 - 3.81 (m, 4H), 3.67 - 3.51 (m, 1H), 3.24 - 3.13 (m, 1H), 3.09 (br d, J = 5.8 Hz, 2H), 3.03 - 3.01 (m, 1H), 2.90 - 2.75 (m, 1H), 2.32 - 2.18 (m, 2H), 2.17 - 1.97 (m, 4H), 1.94 - 1.66 (m, 6H), 0.85 - 0.71 (m, 3H). |
| 9- 36 | 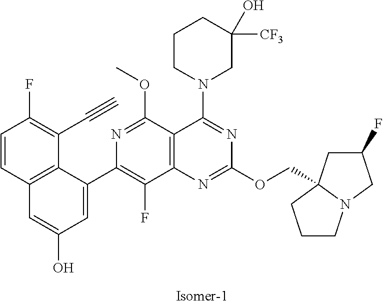 [SMILES: C#Cc1c(F)ccc2cc(O)cc(-c3nc(OC)c4c(N5CCCC(O)(C(F)(F)F)C5)nc(OC[C@@]56CCCN5C[C@H](F)C6)nc4c3F)c12] | 1-(7-(8-ethynyl- 7-fluoro-3- hydroxynaphtha- len-1-yl)-8- fluoro-2- (((2R,7aS)-2- fluorotetrahydro- 1H-pyrrolizin- 7a(5H)- yl)methoxy)-5- methoxypyrido[4, 3-d]pyrimidin- 4-yl)-3- (trifluoromethyl) piperidin-3-ol | 688.5 | ¹H NMR (400 MHz, DMSO- d₆) δ ppm = 10.23 - 10.10 (m, 1H), 8.02 - 7.95 (m, 1H), 7.46 (s, 1H), 7.40 (d, J = 2.8 Hz, 1H), 7.29 (d, J = 2.5 Hz, 1H), 6.34 (s, 1H), 5.43 - 5.15 (m, 1H), 4.55 - 4.44 (m, 1H), 4.13 - 4.04 (m, 2H), 3.95 - 3.80 (m, 5H), 3.67 - 3.59 (m, 1H), 3.22 - 3.05 (m, 4H), 2.87 - 2.79 (m, 1H), 2.21 - 2.02 (m, 4H), 1.93 - 1.67 (m, 6H). |
| 9- 37 | 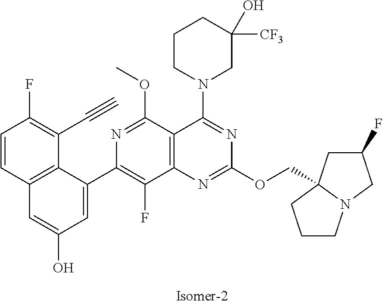 [SMILES: C#Cc1c(F)ccc2cc(O)cc(-c3nc(OC)c4c(N5CCCC(O)(C(F)(F)F)C5)nc(OC[C@@]56CCCN5C[C@H](F)C6)nc4c3F)c12] | 1-(7-(8-ethynyl- 7-fluoro-3- hydroxynaphtha- len-1-yl)-8- fluoro-2- (((2R,7aS)-2- fluorotetrahydro- 1H-pyrrolizin- 7a(5H)- yl)methoxy)-5- methoxypyrido[4, 3-d]pyrimidin- 4-yl)-3- (trifluoromethyl) piperidin-3-ol | 688.5 | ¹H NMR (400 MHz, DMSO- d₆) δ ppm = 10.27 - 10.07 (brs, 1H), 7.98 (dd, J = 9.0, 6.0 Hz, 1H), 7.51 - 7.43 (m, 1H), 7.41 - 7.37 (m, 1H), 7.30 - 7.17 (m, 1H), 6.35 - 6.05 (m, 1H), 5.40 - 5.16 (m, 1H), 4.56 - 4.42 (m, 1H), 4.17 - 3.94 (m, 3H), 3.89 (s, 3H), 3.82 (s, 1H), 3.68 - 3.58 (m, 1H), 3.20 - 3.02 (m, 4H), 2.89 - 2.78 (m, 1H), 2.19 - 1.95 (m, 4H), 1.94 - 1.63 (m, 6H). |
| 9- 38 | 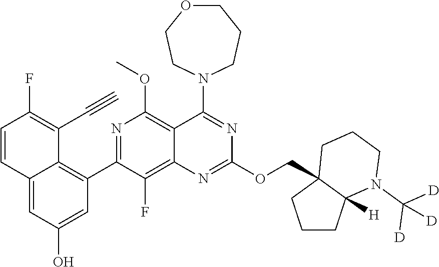 [SMILES: [2H]C([2H])([2H])N1CCC[C@@]2(COc3nc(N4CCCOCC4)c4c(OC)nc(-c5cc(O)cc6ccc(F)c(C#C)c56)c(F)c4n3)CCC[C@@H]12] | 5-ethynyl-6- fluoro-4-(8- fluoro-5- methoxy-2- (((4aS,7aR)-1- (methyl- d3)octahydro- 4aH- cyclopenta[b]pyr- idin-4a- yl)methoxy)-4- (1,4-oxazepan-4- yl)pyrido[4,3- d]pyrimidin-7- yl)naphthalen-2- ol | 631.7 | ¹H NMR (400 MHz, DMSO- d₆) δ ppm = 10.15 (s, 1H), 7.97 (dd, J = 9.1, 5.9 Hz, 1H), 7.47 (t, J = 9.0 Hz, 1H), 7.39 (d, J = 2.8 Hz, 1H), 7.23 (d, J = 2.3 Hz, 1H), 4.43 (br d, J = 10.8 Hz, 1H), 4.19 - 4.09 (m, 1H), 4.02 (dd, J = 1.9, 1.1 Hz, 1H), 3.89 (s, 3H), 3.92 - 3.80 (m, 6H), 3.77 - 3.70 (m, 2H), 2.68 - 2.62 (m, 1H), 2.47 - 2.41 (m, 1H), 2.29 - 2.20 (m, 1H), 2.07 - 2.01 (m, 2H), 1.90 - 1.68 (m, 2H), 1.66 - 1.50 (m, 7H), 1.42 - 1.39 (m, 1H). |
| 9- 39 | 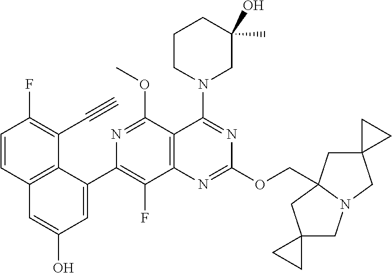 [SMILES: C#Cc1c(F)ccc2cc(O)cc(-c3nc(OC)c4c(N5CCC[C@@](C)(O)C5)nc(OCC56CC7(CC7)CN5CC5(CC5)C6)nc4c3F)c12] | (R)-1-(2- ((1′H,3′H,5′H- dispiro[cyclopro- pane-1,2′- pyrrolizine-6′,1″- cyclopropan]- 7a′(7′H)- yl)methoxy)-7- (8-ethynyl-7- fluoro-3- hydroxynaphtha- len-1-yl)-8- fluoro-5- methoxypyrido[4, 3-d]pyrimidin- 4-yl)-3- methylpiperidin- | 668.3 | ¹H NMR (400 MHz, DMSO- d₆) δ ppm = 10.74 - 9.55 (m, 1H), 7.97 (dd, J = 9.2, 5.9 Hz, 1H), 7.50 - 7.43 (m, 1H), 7.39 (d, J = 2.5 Hz, 1H), 7.30 - 7.20 (m, 1H), 4.62 - 4.50 (m, 1H), 4.50 - 4.44 (m, 1H), 4.46 - 4.28 (m, 2H), 4.26 - 4.19 (m, 1H), 3.98 - 3.85 (m, 3H), 3.55 - 3.42 (m, 2H), 3.14 - 3.04 (m, 1H), 2.83 - 2.70 (m, 4H), 2.11 - 1.96 (m, 1H), 1.93 - 1.86 (m, 2H), 1.83 - 1.75 (m, 2H), 1.72 - 1.50 (m, 3H), 1.20 - 1.11 (m, 3H), 0.60 - 0.43 (m, 8H). |
| | | 3-ol | | |
| 9- 40 | 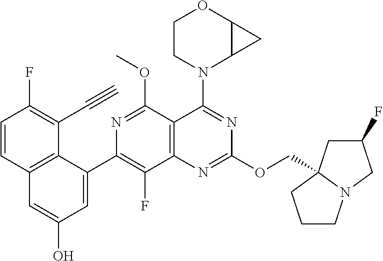 [SMILES: C#Cc1c(F)ccc2cc(O)cc(-c3nc(OC)c4c(N5CCOC6CC65)nc(OC[C@@]56CCCN5C[C@H](F)C6)nc4c3F)c12] | 4-(4-(2-oxa-5- azabicyclo[4.1.0] heptan-5-yl)-8- fluoro-2- (((2R,7aS)-2- fluorotetrahydro- 1H-pyrrolizin- 7a(5H)- yl)methoxy)-5- methoxypyrido[4, 3-d]pyrimidin- 7-yl)-5-ethynyl- 6- fluoronaphthalen- 2-ol | 618.2 | ¹H NMR (400 MHz, DMSO- d₆) δ ppm = 10.26 - 10.05 (m, 1H), 8.02 - 7.93 (m, 1H), 7.46 (td, J = 9.0, 2.2 Hz, 1H), 7.39 (d, J = 2.1 Hz, 1H), 7.24 (dd, J = 8.4, 2.4 Hz, 1H), 5.41 - 5.18 (m, 1H), 4.27 - 4.00 (m, 4H), 3.90 (d, J = 5.5 Hz, 3H), 3.84 - 3.64 (m, 3H), 3.60 - 3.39 (m, 2H), 3.19 - 3.00 (m, 3H), 2.89 - 2.78 (m, 1H), 2.19 - 1.95 (m, 3H), 1.91 - 1.70 (m, 3H), 0.96 - 0.63 (m, 2H). |
| 9- 41 | 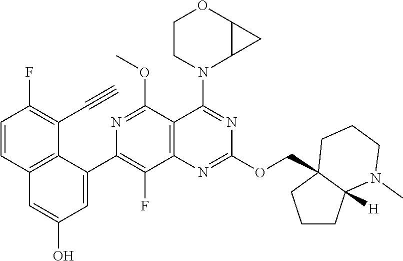 [SMILES: C#Cc1c(F)ccc2cc(O)cc(-c3nc(OC)c4c(N5CCOC6CC65)nc(OC[C@]56CCC[C@H]5N(C)CCC6)nc4c3F)c12] | 4-(4-(2-oxa-5- azabicyclo[4.1.0] heptan-5-yl)-8- fluoro-5- methoxy-2- (((4aS,7aR)-1- methyloctahydro- 4aH- cyclopenta[b]pyr- idin-4a- yl)methoxy)pyri- do[4,3- d]pyrimidin-7- yl)-5-ethynyl-6- fluoronaphthalen- 2-ol | 628.2 | ¹H NMR (400 MHz, DMSO- d₆) δ ppm = 10.13 (br s, 1H), 7.97 (t, J = 6.9 Hz, 1H), 7.46 (dt, J = 9.0, 3.0 Hz, 1H), 7.4 - 7.4 (m, 1H), 7.23 (dd, J = 7.5, 2.3 Hz, 1H), 4.43 (dd, J = 10.6, 3.4 Hz, 1H), 4.20 - 4.11 (m, 1H), 4.06 (d, J = 4.1 Hz, 1H), 3.89 (s, 1H), 3.93 - 3.82 (m, 3H), 3.81 - 3.62 (m, 3H), 3.52 - 3.41 (m, 2H), 3.1- 3.2 (m, 1H), 2.57 - 2.54 (m, 1H), 2.1- 2.2 (m, 4H), 1.83 - 1.55 (m, 2H), 1.5- 1.6 (m, 8H), 0.9- 0.6 (m, 2H). |
| 9- 42 | 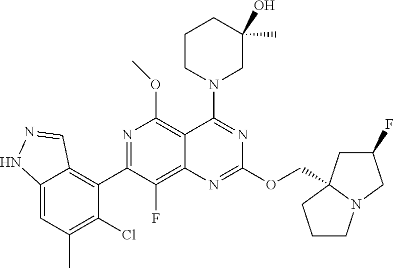 [SMILES: COc1nc(-c2c(Cl)c(C)cc3[nH]ncc23)c(F)c2nc(OC[C@@]34CCCN3C[C@H](F)C4)nc(N3CCC[C@@](C)(O)C3)c12] | (3R)-1-(7-(5- chloro-6-methyl- 1H-indazol-4- yl)-8-fluoro-2- (((2R,7aS)-2- fluorotetrahydro- 1H-pyrrolizin- 7a(5H)- yl)methoxy)-5- methoxypyrido[4, 3-d]pyrimidin- 4-yl)-3- methylpiperidin- 3-ol | 614.2 | ¹H NMR (400 MHz, DMSO- d₆) δ ppm = 13.29 (br s, 1H), 7.87 (d, J = 13.3 Hz, 1H), 7.68 (s, 1H), 5.40 - 5.15 (m, 1H), 4.61 - 4.36 (m, 1H), 4.18 - 4.09 (m, 1H), 4.07 - 3.91 (m, 4H), 3.82 - 3.46 (m, 2H), 3.44 - 3.39 (m, 1H), 3.15 - 3.06 (m, 2H), 3.02 (s, 1H), 2.90 - 2.75 (m, 1H), 2.55 (s, 3H), 2.17 -2.11 (m, 1H), 2.09 - 1.94 (m, 3H), 1.91 - 1.71 (m, 4H), 1.68 - 1.51 (m, 3H), 1.09 (d, J = 2.3 Hz, 3H). |

### Preparation of Intermediate 106: (R)-1-(2,7-dichloro-8-fluoropyrido[4,3-d]pyrimidin-4-yl)-3-methylpiperidin-3-ol

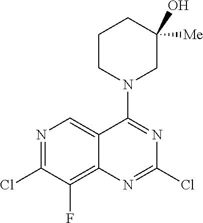 [SMILES: C[C@@]1(O)CCCN(c2nc(Cl)nc3c(F)c(Cl)ncc23)C1]

To a stirred solution of commercially available 2,4,7-trichloro-8-fluoropyrido[4,3-d]pyrimidine (1.5 g, 5.9 mmol) in DCM (24 mL) at 0° C. was added DIPEA (5.2 mL, 30 mmol) followed by (R)-3-methylpiperidin-3-ol (684 mg, 5.9 mmol) dropwise as a solution in DCM (6 mL). The reaction mixture was stirred at 0° C. for 30 minutes. The reaction mixture was quenched with water and extracted with DCM. The combined organic layers were washed with brine, dried over anhydrous magnesium sulfate, filtered, and evaporated under reduced pressure to provide the crude product. The crude compound was purified by COMBIFLASH™ chromatography (Teledyne ISO, Lincoln, NE) (24 g silica gel column, using 0 to 50% ethyl acetate/hexanes) to provide (R)-1-(2,7-dichloro-8-fluoropyrido[4,3-d]pyrimidin-4-yl)-3-methylpiperidin-3-ol (1.61 g, 4.8 mmol, 82% yield) as a pale-yellow solid. MS(ESI) m/z: 332.7 [M+H]⁺. ¹H NMR (400 MHz, CDCl₃) δ=9.08 (s, 1H), 4.64-4.54 (m, 1H), 4.48-4.39 (m, 1H), 3.36 (d, J=13.5 Hz, 2H), 2.20-2.07 (m, 1H), 2.05-2.00 (m, 1H), 1.96-1.88 (m, 1H), 1.86-1.69 (m, 2H), 1.63-1.56 (m, 3H).

### Preparation of Intermediate 107: (R)-1-(7-chloro-8-fluoro-2-(((4aS,7aR)-1-methyloctahydro-4aH-cyclopenta[b]pyridin-4a-yl)methoxy)pyrido[4,3-d]pyrimidin-4-yl)-3-methylpiperidin-3-ol

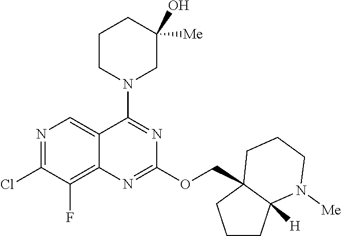 [SMILES: CN1CCC[C@@]2(COc3nc(N4CCC[C@@](C)(O)C4)c4cnc(Cl)c(F)c4n3)CCC[C@@H]12]

To a stirred solution of (R)-1-(2,7-dichloro-8-fluoropyrido[4,3-d]pyrimidin-4-yl)-3-methylpiperidin-3-ol (1.61 g, 4.9 mmol) and ((4aS,7aR)-1-methyloctahydro-4aH-cyclopenta[b]pyridin-4a-yl)methanol (864 mg, 5.1 mmol) in THE (12 mL) at 0° C. was added lithium bis(trimetylsilyl)amide (12.2 mL, 1M in THF, 12.2 mmol). The reaction was allowed to slowly warm to room temperature and stir overnight. Upon completion, the reaction mixture was diluted in ethyl acetate and partitioned with saturated aq. NH₄Cl and subsequently extracted with ethyl acetate. The combined organic layers were washed with brine, dried over anhydrous magnesium sulfate, filtered, and evaporated under reduced pressure to provide the crude product. The crude compound was purified by COMBIFLASH™ chromatography (Teledyne ISO, Lincoln, NE) (40 g silica gel column, using 0 to 20% A:B where A=10% 7N NH3 in MeOH DCM and B=DCM) to provide (R)-1-(7-chloro-8-fluoro-2-(((4aS,7aR)-1-methyloctahydro-4aH-cyclopenta[b]pyridin-4a-yl)methoxy)pyrido[4,3-d]pyrimidin-4-yl)-3-methylpiperidin-3-ol (1.462 g, 3.15 mmol, 65% yield) as a yellow fluffy solid. MS(ESI) m/z: 464.1 [M+H]⁺. ¹H NMR (400 MHz, CDCl₃) δ=8.99-8.83 (m, 1H), 4.49 (d, J=10.7 Hz, 1H), 4.45-4.35 (m, 2H), 4.33 (d, J=10.7 Hz, 1H), 3.49-3.35 (m, 1H), 3.32-3.24 (m, 1H), 2.77-2.67 (m, 1H), 2.64-2.54 (m, 1H), 2.29 (s, 4H), 2.13-1.63 (m, 14H), 1.56-1.48 (m, 1H), 1.36 (s, 3H).

### Preparation of Intermediate 108: 3-bromo-4-isopropylphenol

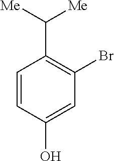 [SMILES: CC(C)c1ccc(O)cc1Br]

A 2-dram vial was charged with isobutyric acid (0.136 mL, 1.500 mmol) and N-hydroxyphthalimide (306 mg, 1.875 mmol). The vial was sealed and purged with nitrogen and the contents were diluted in anhydrous THE (0.75 mL). To this solution was added N,N′-diisopropylcarbodiimide (0.260 mL, 1.650 mmol) dropwise via syringe. The resulting solution was allowed to stir at room temperature for 1 hour. A second 2-dram vial was charged with nickel chloride hexahydrate (95 mg, 0.400 mmol), 2,2′-bipyridine (62.5 mg) and 3-bromo-4-iodophenol (299 mg, 1.000 mmol). The vial was sealed and purged with nitrogen and the contents were diluted in NMP (3.75 mL). The contents were allowed to stir until homogenous. A 5 mL ElectraSyn 2.0 (IKA Works, Inc., Wilmington, NC) vial equipped with a stir bar was charged with silver nitrate (85 mg, 0.500 mmol). The vial was sealed with an ElectraSyn 2.0 cap fitted with a magnesium anode and a reticulated vitreous carbon cathode. The vial was purged with nitrogen three times and placed under inert atmosphere. The two solutions containing the acid starting material and the nickel catalyst were combined and added to the sealed ElectraSyn vial via syringe. The contents were allowed to undergo electrolysis at 30 mA for 3.0 F/mol. Upon completion, the reaction mixture was diluted in ethyl acetate and partitioned between water. The aqueous material was extracted with ethyl acetate. The combined organic layers were washed with brine, dried over anhydrous magnesium sulfate, filtered, and evaporated under reduced pressure to provide the crude product. The crude compound was purified by COMBIFLASH™ chromatography (Teledyne ISO, Lincoln, NE) (24 g silica gel column, using 0 to 5% ethyl acetate/hexanes) to provide 3-bromo-4-isopropylphenol (24 mg, 0.110 mmol, 11% yield) as a clear colorless oil. ¹H NMR (400 MHz, CDCl₃) δ=7.16 (d, J=8.5 Hz, 1H), 7.07 (d, J=2.8 Hz, 1H), 6.79 (dd, J=8.5, 2.5 Hz, 1H), 3.31 (s, 1H), 1.25-1.21 (m, 6H).

### Preparation of Intermediate 109: 4-isopropyl-3-(4,4,5,5-tetramethyl-1,3,2-dioxaborolan-2-yl)phenol

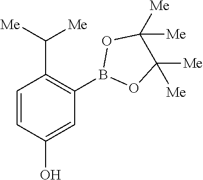 [SMILES: CC(C)c1ccc(O)cc1B1OC(C)(C)C(C)(C)O1]

A 2-dram vial was charged with 3-bromo-4-isopropylphenol (12.1 mg, 0.056 mmol), 4,4,4′,4′,5,5,5′,5′-octamethyl-2,2′-bi(1,3,2-dioxaborolane) (28.3 mg, 0.112 mmol), (1,1′-bis(diphenylphosphino)ferrocene)palladium(II) dichloride (4.1 mg, 0.006 mmol), and potassium acetate (16.4 mg, 0.167 mmol). The vial was sealed and purged with nitrogen. The contents of the vial were diluted in 1,4-dioxane (0.279 mL). The reaction mixture was allowed to stir overnight at 90° C. Upon completion, the reaction mixture was diluted in ethyl acetate and filtered through celite. The resulting filtrate was concentrated to afford the crude product as a dark brown paste. The crude material containing 4-isopropyl-3-(4,4,5,5-tetramethyl-1,3,2-dioxaborolan-2-yl)phenol (40 mg crude, ˜35% pure, 96% yield) was used in the subsequent step without any additional purification. ¹H NMR (500 MHz, CDCl₃) δ=7.21 (s, 2H), 6.91 (s, 1H), 3.67-3.58 (m, 1H), 1.36 (s, 12H), 1.24-1.21 (m, 6H).

### Preparation of Intermediate 110: 3-bromo-4-cyclobutylphenol

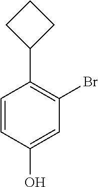 [SMILES: Oc1ccc(C2CCC2)c(Br)c1]

A 2-dram vial was charged with cyclobutanecarboxylic acid (150 mg, 1.500 mmol) and N-hydroxyphthalimide (306 mg, 1.875 mmol). The vial was sealed and purged with nitrogen and the contents were diluted in anhydrous THE (0.75 mL). To this solution was added N,N′diisopropylcarbodiimide (0.260 mL, 1.650 mmol) dropwise via syringe. The resulting solution was allowed to stir at room temperature for 1 hour. A second 2-dram vial was charged with nickel chloride hexahydrate (95 mg, 0.400 mmol), 2,2′-bipyridine (62.5 mg) and 3-bromo-4-iodophenol (299 mg, 1.000 mmol). The vial was sealed and purged with nitrogen and the contents were diluted in NMP (3.75 mL). The contents were allowed to stir until homogenous. A 5 mL IKA ElectraSyn 2.0 vial equipped with a stir bar was charged with silver nitrate (85 mg, 0.500 mmol). The vial was sealed with an IKA ElectraSyn 2.0 cap fitted with a magnesium anode and a reticulated vitreous carbon cathode. The vial was purged with nitrogen three times and placed under inert atmosphere. The two solutions containing the acid starting material and the nickel catalyst were combined and added to the sealed electrasyn vial via syringe. The contents were allowed to undergo electrolysis at 30 mA for 3.0 F/mol. Upon completion, the reaction mixture was diluted in ethyl acetate and partitioned between water. The aqueous material was extracted with ethyl acetate. The combined organic layers were washed with brine, dried over anhydrous magnesium sulfate, filtered, and evaporated under reduced pressure to provide the crude product. The crude compound was purified by COMBIFLASH™ chromatography (Teledyne ISO, Lincoln, NE) (24 g silica gel column, using 0 to 5% ethyl acetate/hexanes) to provide 3-bromo-4-cyclobutylphenol (28 mg, 0.123 mmol, 12% yield) as a clear colorless oil. ¹H NMR (400 MHz, CDCl₃) δ=7.38-7.34 (m, 1H), 7.18-7.13 (m, 1H), 6.76-6.74 (m, 1H), 3.91-3.68 (m, 1H), 2.47 (br d, J=8.5 Hz, 2H), 2.21-2.11 (m, 2H), 2.10-2.00 (m, 1H), 1.95-1.84 (m, 1H).

### Preparation of Intermediate 111: 4-cyclobutyl-3-(4,4,5,5-tetramethyl-1,3,2-dioxaborolan-2-yl)phenol

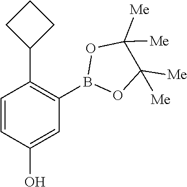 [SMILES: CC1(C)OB(c2cc(O)ccc2C2CCC2)OC1(C)C]

A 2-dram vial was charged with 3-bromo-4-cyclobutylphenol (20.0 mg, 0.078 mmol), 4,4,4′,4′,5,5,5′,5′-octamethyl-2,2′-bi(1,3,2-dioxaborolane) (39.5 mg, 0.156 mmol), (1,1′-bis(diphenylphosphino)ferrocene)palladium(II) dichloride (5.7 mg, 0.008 mmol), and potassium acetate (22.9 mg, 0.233 mmol). The vial was sealed and purged with nitrogen. The contents of the vial were diluted in 1,4-dioxane (0.4 mL). The reaction mixture was allowed to stir overnight at 90° C. Upon completion, the reaction mixture was diluted in ethyl acetate and filtered through celite. The resulting filtrate was concentrated to afford the crude product as a dark brown paste. The crude material containing 4-cyclobutyl-3-(4,4,5,5-tetramethyl-1,3,2-dioxaborolan-2-yl)phenol (50 mg crude, −40% pure, 85% yield) was used in the subsequent step without any additional purification.

### Preparation of Intermediate 112: 4-(bicyclo[1.1.1]pentan-1-yl)-3-bromophenol

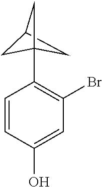 [SMILES: Oc1ccc(C23CC(C2)C3)c(Br)c1]

A 2-dram vial was charged with bicyclo[1.1.1]pentane-1-carboxylic acid (100 mg, 0.892 mmol) and N-hydroxyphthalimide (182 mg, 1.115 mmol). The vial was sealed and purged with nitrogen and the contents were diluted in anhydrous THF (0.75 mL). To this solution was added N,N′diisopropylcarbodiimide (0.154 mL, 0.891 mmol) dropwise via syringe. The resulting solution was allowed to stir at room temperature for 1 hour. A second 2-dram vial was charged with nickel chloride hexahydrate (42.2 mg, 0.178 mmol), 2,2′-bipyridine (27.9 mg) and 3-bromo-4-iodophenol (400 mg, 1.338 mmol). The vial was sealed and purged with nitrogen and the contents were diluted in NMP (3.75 mL). The contents were allowed to stir until homogenous. A 5 mL IKA ElectraSyn 2.0 vial equipped with a stir bar was charged with silver nitrate (76 mg, 0.446 mmol). The vial was sealed with an IKA ElectraSyn 2.0 cap fitted with a magnesium anode and a reticulated vitreous carbon cathode. The vial was purged with nitrogen three times and placed under inert atmosphere. The two solutions containing the acid starting material and the nickel catalyst were combined and added to the sealed electrasyn vial via syringe. The contents were allowed to undergo electrolysis at 30 mA for 3.0 F/mol. Upon completion, the reaction mixture was diluted in ethyl acetate and partitioned between water. The aqueous material was extracted with ethyl acetate. The combined organic layers were washed with brine, dried over anhydrous magnesium sulfate, filtered, and evaporated under reduced pressure to provide the crude product. The crude compound was purified by COMBIFLASH™ chromatography (Teledyne ISO, Lincoln, NE) (24 g silica gel column, using 0 to 5% ethyl acetate/hexanes) to provide 4-(bicyclo[1.1.1]pentan-1-yl)-3-bromophenol (38 mg, 0.161 mmol, 18% yield) as a clear colorless oil. ¹H NMR (400 MHz, CDCl₃) δ=7.07-7.00 (m, 2H), 6.75-6.70 (m, 1H), 2.55-2.53 (m, 1H), 2.26 (s, 6H).

### Preparation of Intermediate 113: 4-(bicyclo[1.1.1]pentan-1-yl)-3-(4,4,5,5-tetramethyl-1,3,2-dioxaborolan-2-yl)phenol

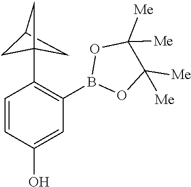 [SMILES: CC1(C)OB(c2cc(O)ccc2C23CC(C2)C3)OC1(C)C]

A 2-dram vial was charged with 4-(bicyclo[1.1.1]pentan-1-yl)-3-bromophenol (38 mg, 0.159 mmol), 4,4,4′,4′,5,5,5′,5′-octamethyl-2,2′-bi(1,3,2-dioxaborolane) (50.4 mg, 0.199 mmol), (1,1′-bis(diphenylphosphino)ferrocene)palladium(II) dichloride (11.6 mg, 0.016 mmol), and potassium acetate (46.8 mg, 0.477 mmol). The vial was sealed and purged with nitrogen. The contents of the vial were diluted in 1,4-dioxane (0.795 mL). The reaction mixture was allowed to stir overnight at 90° C. Upon completion, the reaction mixture was diluted in ethyl acetate and filtered through celite. The resulting filtrate was concentrated to afford the crude product as a dark brown paste. The crude material containing 4-(bicyclo[1.1.1]pentan-1-yl)-3-(4,4,5,5-tetramethyl-1,3,2-dioxaborolan-2-yl)phenol (37 mg crude, 81% yield) was used in the subsequent step without any additional purification. ¹H NMR (500 MHz, CDCl₃) δ=7.17-6.97 (m, 2H), 6.77 (br d, J=8.7 Hz, 1H), 2.22-2.17 (m, 1H), 2.07 (s, 6H), 1.30 (s, 12H).

### Example 10-1

### (3R)-1-(2-{[(4aS,7aR)-1-methyl-octahydro-1H-cyclopenta[b]pyridin-4a-yl]methoxy}-8-fluoro-7-(3-hydroxynaphthalen-1-yl)pyrido[4,3-d]pyrimidin-4-yl)-3-methylpiperidin-3-ol

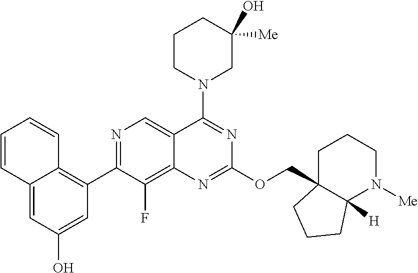 [SMILES: CN1CCC[C@@]2(COc3nc(N4CCC[C@@](C)(O)C4)c4cnc(-c5cc(O)cc6ccccc56)c(F)c4n3)CCC[C@@H]12]

To a 1-dram vial equipped with a stir bar was added (R)-1-(7-chloro-8-fluoro-2-(((4aS,7aR)-1-methyloctahydro-4aH-cyclopenta[b]pyridin-4a-yl)methoxy)pyrido[4,3-d]pyrimidin-4-yl)-3-methylpiperidin-3-ol (20 mg, 0.043 mmol), 4-(4,4,5,5-tetramethyl-1,3,2-dioxaborolan-2-yl)naphthalen-2-ol (14 mg, 0.052 mmol), (1,1′-bis(diphenylphosphino)ferrocene)palladium(II) dichloride (3.2 mg, 0.004 mmol) and sodium carbonate monohydrate (16 mg, 0.130 mmol) directly as solids. The vial was sealed with a polytetrafluoroethylene (PTFE) cap and purged with nitrogen three times. The contents were diluted in 1,4-dioxane (0.345 mL) and water (0.086 mL) and the resulting suspension was allowed to warm to 90° C. and stir overnight. Upon completion, the reaction mixture was diluted in ethyl acetate, partitioned between water and subsequently extracted with ethyl acetate. The combined organics were washed with brine, dried over magnesium sulfate, filtered, and concentrated under reduced pressure to afford the crude product as a brown oil. The crude material was purified via preparative Reverse Phase chromatography with the following conditions: Column: XBridge C18, 19 mm×200 mm, 5 m particles; Flow Rate: 20 mL/min; Column Temperature: 25° C. Mobile phase A: acetonitrile/water (5:95) with 10 mM ammonium acetate; Mobile phase B: acetonitrile/water (95:5) with 10 mM ammonium acetate; Gradient=0 (min)−15% B, 20-55% B, 20.1-100% B, 24-100% B. Fraction collection was triggered by MS (ESI+). Fractions containing the desired product were combined and dried via centrifugal evaporation to afford (3R)-1-(2-{[(4aS,7aR)-1-methyl-octahydro-1H-cyclopenta[b]pyridin-4a-yl]methoxy}-8-fluoro-7-(3-hydroxynaphthalen-1-yl)pyrido[4,3-d]pyrimidin-4-yl)-3-methylpiperidin-3-ol (11.9 mg, 0.021 mmol, 48%). MS(ESI) m/z: 572.1 [M+H]⁺. 1H NMR (500 MHz, DMSO-d6) δ=9.30 (s, 1H), 7.81 (d, J=8.4 Hz, 1H), 7.55 (s, 1H), 7.45 (s, 1H), 7.35-7.21 (m, 3H), 4.56-4.44 (m, 1H), 4.42-4.32 (m, 1H), 4.23-4.13 (m, 1H), 4.12-4.03 (m, 1H), 3.64-3.53 (m, 1H), 3.18 (s, 2H), 2.43-2.09 (m, 3H), 2.09-1.98 (m, 1H), 1.91 (s, 2H), 1.96-1.80 (m, 1H), 1.79-1.36 (m, 12H), 1.19 (s, 3H).

TABLE 7

| | | | LCMS | |
|---|---|---|---|---|
| # | Structure | IUPAC | (M + H)⁺ | ¹H NMR |
| 10-2 | 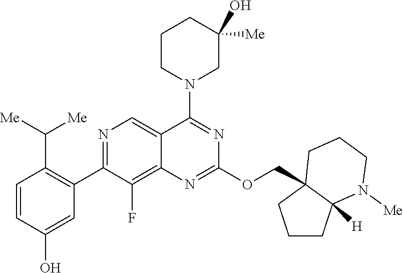 [SMILES: CC(C)c1ccc(O)cc1-c1ncc2c(N3CCC[C@@](C)(O)C3)nc(OC[C@]34CCC[C@H]3N(C)CCC4)nc2c1F] | (3R)-1-(2-{[(4aS,7aR)-1- methyl-octahydro-1H- cyclopenta[b]pyridin-4a- yl]methoxy}-8-fluoro-7- [5-hydroxy-2-(propan-2- yl)phenyl]pyrido[4,3- d]pyrimidin-4-yl)-3- methylpiperidin-3-ol | 564.2 | ¹H NMR (500 MHz, DMSO-d₆) δ ppm = 9.15 (d, J = 4.5 Hz, 1H), 7.22- 7.17 (m, 1H), 6.81-6.76 (m, 1H), 6.56 (s, 1H), 4.60-4.44 (m, 1H), 4.35- 4.14 (m, 2H), 4.12- 4.03 (m, 1H), 4.02-3.92 (m, 1H), 3.82 (s, 1H), 3.74-3.39 (m, 1H), 3.08 (s, 2H), 3.07-2.96 (m, 1H), 2.77-2.54 (m, 4H), 2.25-1.32 (m, 14H), 1.22-0.86 (m, 8H) |
| 10-3 | 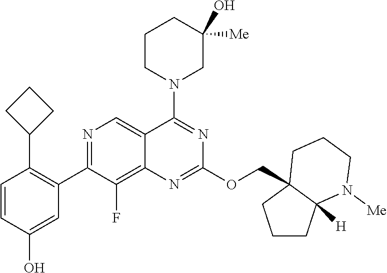 [SMILES: CN1CCC[C@@]2(COc3nc(N4CCC[C@@](C)(O)C4)c4cnc(-c5cc(O)ccc5C5CCC5)c(F)c4n3)CCC[C@@H]12] | (3R)-1-(2-{[(4aS,7aR)-1- methyl-octahydro-1H- cyclopenta[b]pyridin-4a- yl]methoxy}-7-(2- cyclobutyl-5- hydroxyphenyl)-8- fluoropyrido[4,3- d]pyrimidin-4-yl)-3- methylpiperidin-3-ol | 576.2 | ¹H NMR (500 MHz, DMSO-d₆) δ ppm = 9.23 (d, J = 3.3 Hz, 1H), 7.30 (d, J = 8.6 Hz, 1H), 6.88 (dd, J = 8.4, 2.4 Hz, 1H), 6.69 (s, 1H), 4.67-4.56 (m, 1H), 4.41-4.24 (m, 2H), 4.21-4.04 (m, 1H), 3.90 (s, 1H), 3.81-3.67 (m, 1H), 3.60-3.53 (m, 1H), 3.41-3.26 (m, 1H), 3.22-3.04 (m, 2H), 3.00- 2.66 (m, 4H), 2.35- 1.57 (m, 19H), 1.55- 1.43 (m, 1H), 1.18 (s, 3H) |
| 10-4 | 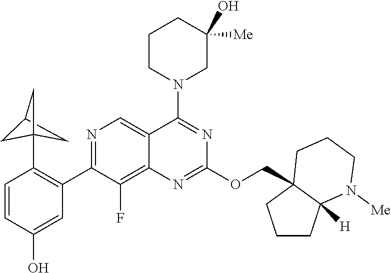 [SMILES: CN1CCC[C@@]2(COc3nc(N4CCC[C@@](C)(O)C4)c4cnc(-c5cc(O)ccc5C56CC(C5)C6)c(F)c4n3)CCC[C@@H]12] | (3R)-1-(2-{[(4aS,7aR)-1- methyl-octahydro-1H- cyclopenta[b]pyridin-4a- yl]methoxy}-7-(2- {bicyclo[1.1.1]pentan-1- yl}-5-hydroxyphenyl)-8- fluoropyrido[4,3- d]pyrimidin-4-yl)-3- methylpiperidin-3-ol | 588.3 | ¹H NMR (500 MHz, DMSO-d₆) δ ppm = 9.27- 9.16 (m, 1H), 7.13- 7.10 (m, 1H), 6.85-6.78 (m, 1H), 6.60 (d, J = 2.3 Hz, 1H), 4.68-4.57 (m, 1H), 4.41-4.23 (m, 2H), 4.20-4.12 (m, 1H), 4.10-4.01 (m, 1H), 3.91 (s, 1H), 3.80- 3.68 (m, 1H), 3.61-3.50 (m, 1H), 3.49-3.31 (m, 1H), 3.17 (s, 2H), 2.90- 2.68 (m, 3H), 2.38- 1.69 (m, 12H), 1.69- 1.66 (m, 6H), 1.56-1.42 (m, 1H), 1.23-1.13 (m, |
| | | | | 3H) |
| 10-5 | 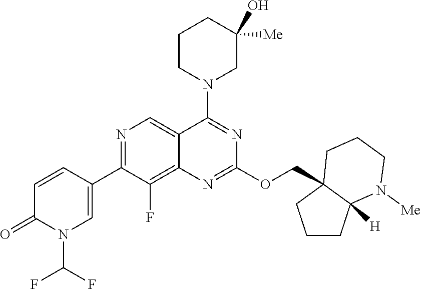 [SMILES: CN1CCC[C@@]2(COc3nc(N4CCC[C@@](C)(O)C4)c4cnc(-c5ccc(=O)n(C(F)F)c5)c(F)c4n3)CCC[C@@H]12] | 5-(2-{[(4aS,7aR)-1- methyl-octahydro-1H- cyclopenta[b]pyridin-4a- yl]methoxy}-8-fluoro-4- [(3R)-3-hydroxy-3- methylpiperidin-1- yl]pyrido[4,3-d]pyrimidin- 7-yl)-1-(difluoromethyl)- 1,2-dihydropyridin-2-one | 573.1 | ¹H NMR (500 MHz, DMSO-d₆) δ ppm = 9.25 (s, 1H), 8.40 (s, 1H), 83.0-8.22 (m, 1H), 8.13- 7.84 (m, 1H), 6.75 (br d, J = 9.2 Hz, 1H), 4.71- 4.13 (m, 3H), 4.10-4.01 (m, 1H), 3.91 (s, 1H), 3.80-3.49 (m, 1H), 3.17 (s, 3H), 3.01-2.87 (m, 1H), 2.86-2.71 (m, 3H), 2.05-1.94 (m, 2H), 1.92-1.59 (m, 11H), 1.56-1.45 (m, 1H), 1.17 (s, 3H) |
| 10-6 | 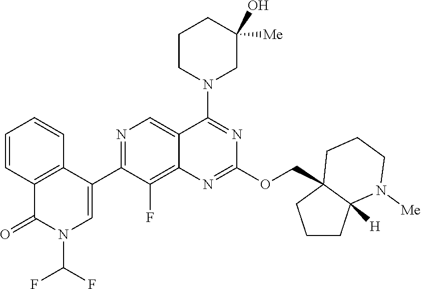 [SMILES: CN1CCC[C@@]2(COc3nc(N4CCC[C@@](C)(O)C4)c4cnc(-c5cn(C(F)F)c(=O)c6ccccc56)c(F)c4n3)CCC[C@@H]12] | 4-(2-{[(4aS,7aR)-1- methyl-octahydro-1H- cyclopenta[b]pyridin-4a- yl]methoxy}-8-fluro-4- [(3R)-3-hydroxy-3- methylpiperidin-1- yl]pyrido[4,3-d]pyrimidin- 7-yl)-2-(difluoromethyl)- 1,2-dihydroisoquinolin-1- one | 623.1 | ¹H NMR (500 MHz, DMSO-d₆) δ ppm = 9.36- 9.30 (m, 1H), 8.45- 8.37 (m, 1H), 8.25-7.94 (m, 1H), 7.89-7.77 (m, 2H), 7.75-7.67 (m, 1H), 7.61-7.52 (m, 1H), 4.69-4.58 (m, 1H), 4.47-4.05 (m, 3H), 4.00-3.84 (m, 1H), 3.80-3.25 (m, 2H), 3.22-2.92 (m, 2H), 2.86-2.70 (m, 3H), 2.31-1.92 (m, 3H), 1.91-1.59 (m, 10H), 1.56-1.43 (m, 1H), 1.19 (s, 3H) |
| 10-7 | 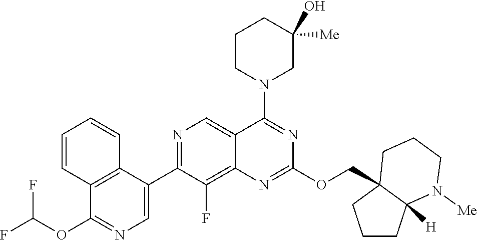 [SMILES: CN1CCC[C@@]2(COc3nc(N4CCC[C@@](C)(O)C4)c4cnc(-c5cnc(OC(F)F)c6ccccc56)c(F)c4n3)CCC[C@@H]12] | (3R)-1-(2-{[(4aS,7aR)-1- methyl-octahydro-1H- cyclopenta[b]pyridin-4a- yl]methoxy}-7-[1- (difluoromethoxy)isoquin- olin-4-yl]-8- fluoropyrido[4,3- d]pyrimidin-4-yl)-3- methylpiperidin-3-ol | 623.1 | ¹H NMR (500 MHz, DMSO-d₆) δ ppm = 9.41- 9.27 (m, 1H), 8.31 (s, 2H), 8.21-7.80 (m, 4H), 4.70-4.04 (m, 4H), 4.00-3.56 (m, 1H), 3.42-3.27 (m, 1H), 3.19-2.92 (m, 2H), 2.87-2.70 (m, 3H), 2.33-1.58 (m, 15H), 1.55-1.43 (m, 1H), 1.19 (s, 3H) |
| 10-8 | 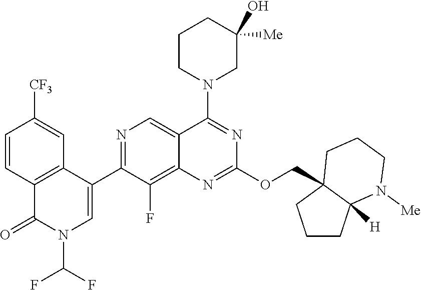 [SMILES: CN1CCC[C@@]2(COc3nc(N4CCC[C@@](C)(O)C4)c4cnc(-c5cn(C(F)F)c(=O)c6ccc(C(F)(F)F)cc56)c(F)c4n3)CCC[C@@H]12] | 4-(2-{[(4aS,7aR)-1- methyl-octahydro-1H- cyclopenta[b]pyridin-4a- yl]methoxy}-8-fluoro-4- [(3R)-3-hydroxy-3- methylpiperidin-1- yl]pyrido[4,3-d]pyrimidin- 7-yl)-2-(difluoromethyl)- 6-(trifluoromethyl)-1,2- dihydroisoquinolin-1-one | 691.2 | |
| 10-9 | 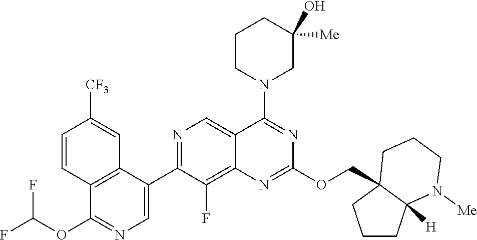 [SMILES: CN1CCC[C@@]2(COc3nc(N4CCC[C@@](C)(O)C4)c4cnc(-c5cnc(OC(F)F)c6ccc(C(F)(F)F)cc56)c(F)c4n3)CCC[C@@H]12] | (3R)-1-(2-{[(4aS,7aR)-1- methyl-octahydro-1H- cyclopenta[b]pyridin-4a- yl]methoxy}-7-[1- (difluoromethxoy)-6- (trifluoromethyl)iso quinolin-4-yl]-8- fluoropyrido[4,3- d]pyrimidin-4-yl)-3- methylpiperidin-3-ol | 691.2 | |

### Preparation of Intermediate 114: (R)-1-(7-bromo-2-chloro-8-fluoroquinazolin-4-yl)-3-methylpiperidin-3-ol

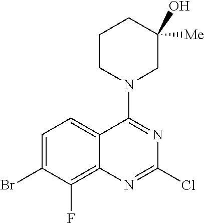 [SMILES: C[C@@]1(O)CCCN(c2nc(Cl)nc3c(F)c(Br)ccc23)C1]

To a stirred solution of commercially available 7-bromo-2,4-dichloro-8-fluoroquinazoline (1.5 g, 5.1 mmol) in DCM (20 mL) at 0° C. was added DIPEA (4.3 mL, 25.3 mmol) followed by (R)-3-methylpiperidin-3-ol (584 mg, 5.1 mmol) dropwise as a solution in DCM (5 mL). The reaction mixture was stirred at 0° C. for 30 minutes. The reaction mixture was quenched with water and extracted with DCM. The combined organic layers were washed with brine, dried over anhydrous magnesium sulfate, filtered, and evaporated under reduced pressure to provide the crude product. The crude compound was purified by COMBIFLASH™ chromatography (Teledyne ISO, Lincoln, NE) (24 g silica gel column, using 0 to 50% ethyl acetate/hexanes) to provide (R)-1-(7-bromo-2-chloro-8-fluoroquinazolin-4-yl)-3-methylpiperidin-3-ol (1.51 g, 4.1 mmol, 80% yield) as a pale-yellow solid. MS(ESI) m/z: 373.7 [M+H]⁺. ¹H NMR (400 MHz, CDCl₃) δ=7.74-7.68 (m, 1H), 7.60-7.51 (m, 1H), 4.40-4.21 (m, 2H), 3.42-3.29 (m, 1H), 3.27-3.19 (m, 1H), 2.11-2.01 (m, 1H), 1.96-1.86 (m, 1H), 1.81-1.62 (m, 2H), 1.35 (s, 3H).

### Preparation of Intermediate 115: (R)-1-(7-bromo-8-fluoro-2-(((4aS,7aR)-1-methyloctahydro-4aH-cyclopenta[b]pyridin-4a-yl)methoxy)quinazolin-4-yl)-3-methylpiperidin-3-ol

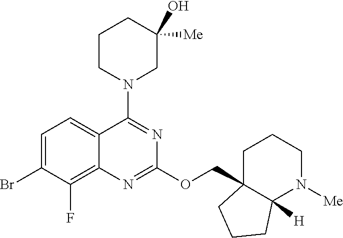 [SMILES: CN1CCC[C@@]2(COc3nc(N4CCC[C@@](C)(O)C4)c4ccc(Br)c(F)c4n3)CCC[C@@H]12]

To a stirred solution of (R)-1-(7-bromo-2-chloro-8-fluoroquinazolin-4-yl)-3-methylpiperidin-3-ol (1.51 g, 4.1 mmol) and ((4aS,7aR)-1-methyloctahydro-4aH-cyclopenta[b]pyridin-4a-yl)methanol (716 mg, 4.2 mmol) in THE (10 mL) at room temperature was added lithium bis(trimetylsilyl)amide (10.1 mL, 1M in THF, 10.1 mmol). The reaction was allowed to slowly warm to 90° C. and stir overnight. Upon completion, the reaction mixture was diluted in ethyl acetate and partitioned with saturated aq. NH₄Cl and subsequently extracted with ethyl acetate. The combined organic layers were washed with brine, dried over anhydrous magnesium sulfate, filtered, and evaporated under reduced pressure to provide the crude product. The crude compound was purified by COMBIFLASH™ chromatography (Teledyne ISO, Lincoln, NE) (40 g silica gel column, using 0 to 25% A:B where A=10% 7N NH3 in MeOH DCM and B=DCM) to provide (R)-1-(7-bromo-8-fluoro-2-(((4aS,7aR)-1-methyloctahydro-4aH-cyclopenta[b]pyridin-4a-yl)methoxy)quinazolin-4-yl)-3-methylpiperidin-3-ol (973 mg, 2.9 mmol, 48% yield) as a yellow fluffy solid. MS(ESI) m/z: 508.9 [M+H]⁺. ¹H NMR (400 MHz, CDCl₃) δ=7.63-7.54 (m, 1H), 7.39-7.32 (m, 1H), 4.53-4.44 (m, 1H), 4.30-4.16 (m, 3H), 3.73-3.63 (m, 1H), 3.40-3.24 (m, 1H), 3.23-3.08 (m, 1H), 2.79-2.52 (m, 2H), 2.29 (s, 4H), 2.04-1.62 (m, 13H), 1.34 (s, 3H).

### Example 11-1

### (3R)-1-(2-{[(4aS,7aR)-1-methyl-octahydro-1H-cyclopenta[b]pyridin-4a-yl]methoxy}-8-fluoro-7-(3-hydroxynaphthalen-1-yl)quinazolin-4-yl)-3-methylpiperidin-3-ol

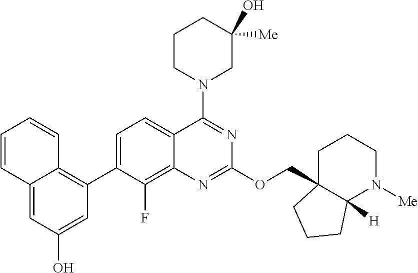 [SMILES: CN1CCC[C@@]2(COc3nc(N4CCC[C@@](C)(O)C4)c4ccc(-c5cc(O)cc6ccccc56)c(F)c4n3)CCC[C@@H]12]

To a 1-dram vial equipped with stir bar was (R)-1-(7-bromo-8-fluoro-2-(((4aS,7aR)-1-methyloctahydro-4aH-cyclopenta[b]pyridin-4a-yl)methoxy)quinazolin-4-yl)-3-methylpiperidin-3-ol (20 mg, 0.039 mmol), 4-(4,4,5,5-tetramethyl-1,3,2-dioxaborolan-2-yl)naphthalen-2-ol (12.8 mg, 0.047 mmol), (1,1′-bis(diphenylphosphino)ferrocene)palladium(II) dichloride (2.9 mg, 0.004 mmol) and sodium carbonate monohydrate (14.7 mg, 0.118 mmol) directly as solids. The vial was sealed with a PTFE cap and purged with nitrogen three times. The contents were diluted in 1,4-dioxane (0.345 mL) and water (0.086 mL) and the resulting suspension was allowed to warm to 90° C. and stir overnight. Upon completion, the reaction mixture was diluted in ethyl acetate, partitioned between water and subsequently extracted with ethyl acetate. The combined organics were washed with brine, dried over magnesium sulfate, filtered, and concentrated under reduced pressure to afford the crude product as a brown oil. The crude material was purified via preparative Reverse Phase chromatography with the following conditions: Column: XBridge C18, 19 mm×200 mm, 5 m particles; Flow Rate: 20 mL/min; Column Temperature: 25° C. Mobile phase A: acetonitrile/water (5:95) with 10 mM ammonium acetate; Mobile phase B: acetonitrile/water (95:5) with 10 mM ammonium acetate; Gradient=0 (min)−11% B, 51% B, 20.1-100% B, 24-100% B. Fraction collection was triggered by MS (ESI+). Fractions containing the desired product were combined and dried via centrifugal evaporation to provide (3R)-1-(2-{[(4aS,7aR)-1-methyl-octahydro-1H-cyclopenta[b]pyridin-4a-yl]methoxy}-8-fluoro-7-(3-hydroxynaphthalen-1-yl)quinazolin-4-yl)-3-methylpiperidin-3-ol (13.0 mg, 0.021 mmol, 55%). MS(ESI) m/z: 571.2 [M+H]⁻. 1H NMR (500 z, DMSO-d6) δ=8.05-7.97 (m, 1H), 7.85-7.78 (m, 1H), 7.49-7.36 (m, 2H), 7.36-7.29 (m, 1H), 7.29-7.20 (m, 3H), 7.14-7.09 (m, 1H), 4.64-4.54 (m, 1H), 4.37-4.24 (m, 1H), 4.21-4.10 (m, 1H), 3.99-3.88 (m, 1H), 3.79-3.70 (m, 1H), 3.55-3.30 (m, 1H), 3.18 (s, 3H), 3.16-3.02 (m, 1H), 2.88-2.69 (i, 3H), 2.34-1.42 (m, 13H), 1.18 (s, 3H).

TABLE 8

| | | | LCMS | |
|---|---|---|---|---|
| # | Structure | IUPAC | (M + H)⁺ | ¹H NMR |
| 11-2 | 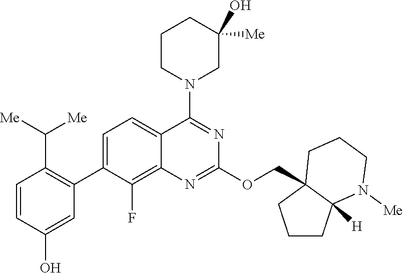 [SMILES: CC(C)c1ccc(O)cc1-c1ccc2c(N3CCC[C@@](C)(O)C3)nc(OC[C@]34CCC[C@H]3N(C)CCC4)nc2c1F] | (3R)-1-(2-{[(4aS,7aR)-1- methyl-octahydro-1H- cyclopenta[b]pyridin-4a- yl]methoxy}-8-fluoro-7- [5-hydroxy-2-(propan-2- yl)phenyl]quinazolin-4- yl)-3-methylpiperidin-3- ol | 563.4 | ¹H NMR (500 MHz, DMSO-d₆) δ ppm = 7.72- 7.61 (m, 1H), 7.12- 7.00 (m, 1H), 6.93-6.84 (m, 1H), 6.67-6.59 (m, 1H), 6.38-6.30 (m, 1H), 4.36-4.14 (m, 1H), 3.98-3.76 (m, 2H), 3.73-3.53 (m, 1H), 2.50-2.37 (m, 2H), 2.26-2.19 (m, 1H), 1.96 (s, 3H), 1.84- 1.11 (m, 18H), 1.03- 0.72 (m, 8H) |
| 11-3 | 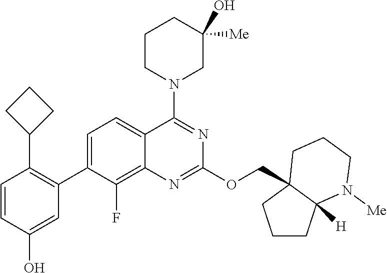 [SMILES: CN1CCC[C@@]2(COc3nc(N4CCC[C@@](C)(O)C4)c4ccc(-c5cc(O)ccc5C5CCC5)c(F)c4n3)CCC[C@@H]12] | (3R)-1-(2-{[(4aS,7aR)-1- methyl-octahydro-1H- cyclopenta[b]pyridin-4a- yl]methoxy}-7-(2- cyclobutyl-5- hydroxyphenyl)-8- fluoroquinazolin-4-yl)-3- methylpiperidin-3-ol | 575.4 | ¹H NMR (500 MHz, DMSO-d₆) δ ppm = 7.97- 7.84 (m, 1H), 7.31 (d, J = 8.5 Hz, 1H), 7.17- 7.07 (m, 1H), 6.91-6.80 (m, 1H), 6.63-6.49 (m, 1H), 4.75- 4.49 (m, 2H), 4.30 (br d, J = 11.0 Hz, 1H), 4.20- 4.02 (m, 2H), 3.93-3.82 (m, 1H), 3.80-3.68 (m, 1H), 3.16-3.07 (m, 2H), 3.02-2.90 (m, 1H), 2.86-2.72 (m, 3H), 2.33- 2.23 (m, 1H), 2.20- 2.09 (m, 1H), 1.65 (br |
| | | | | m, 17H), 1.57-1.46 (m, |
| | | | | 1H), 1.17 (s, 3H) |

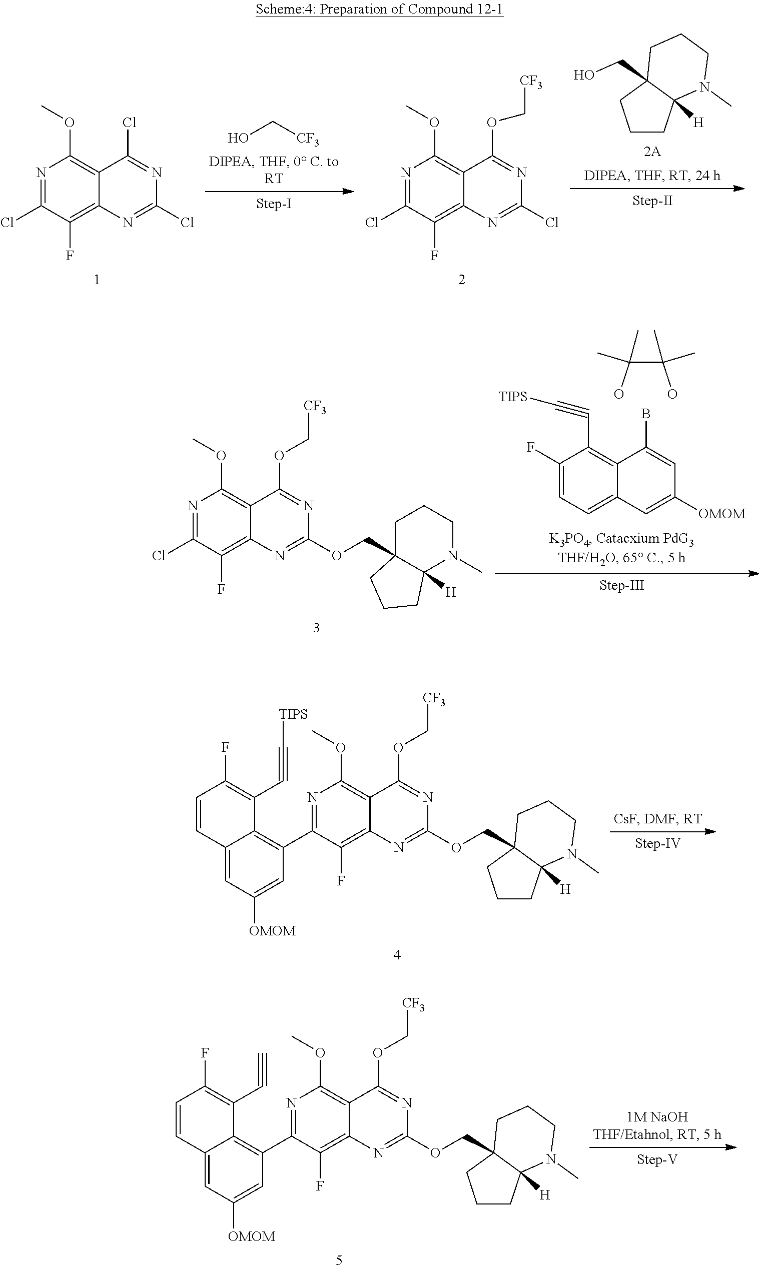

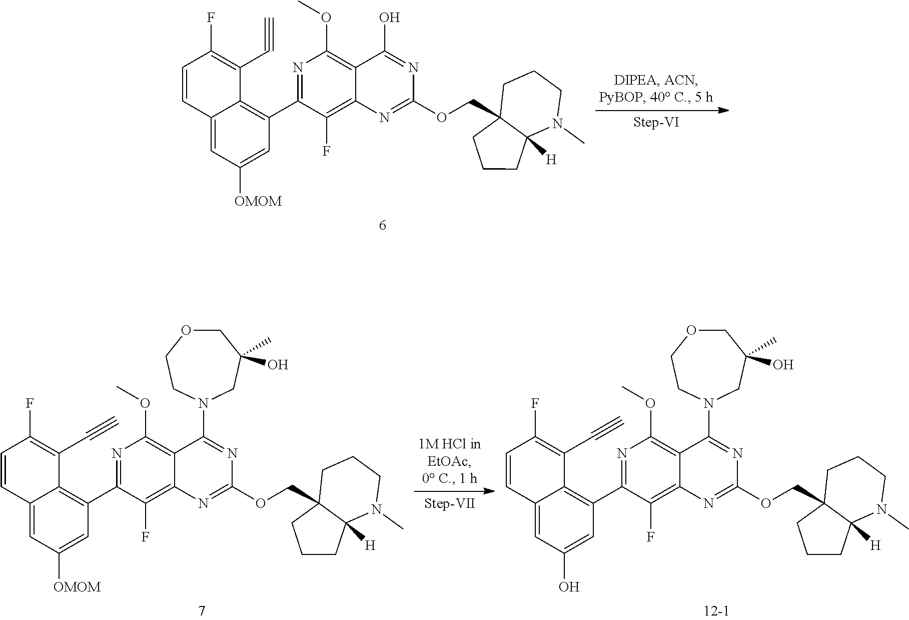

### Preparation of Intermediate 2: 2,7-dichloro-8-fluoro-5-methoxy-4-(2,2,2-trifluoroethoxy)pyrido[4,3-d]pyrimidine

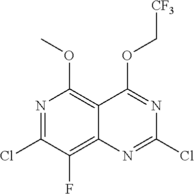 [SMILES: COc1nc(Cl)c(F)c2nc(Cl)nc(OCC(F)(F)F)c12]

### Preparation of Intermediate 116: 2,7-dichloro-8-fluoro-5-methoxy-4-(2,2,2-trifluoroethoxy)pyrido[4,3-d]pyrimidine

To a stirred suspension of 2,4,7-trichloro-8-fluoro-5-methoxypyrido[4,3-d]pyrimidine (2.0 g, 7.08 mmol) and molecular sieves (250 mg) in THE (40 mL) at 0° C. were added DIPEA (2.47 mL, 14.16 mmol) and 2,2,2-trifluoroethan-1-ol (3.06 mL, 42.5 mmol) with an interval of 5 min. The resulting solution was gradually warmed up to room temperature over a period of 18 h. The reaction mixture was quenched with water and extracted with ethyl acetate (100 mL). The organic layer was washed with brine, dried over anhydrous sodium sulfate, filtered and evaporated under reduced pressure to provide 2,7-dichloro-8-fluoro-5-methoxy-4-(2,2,2-trifluoroethoxy)pyrido[4,3-d]pyrimidine (1.8 g, 3.74 mmol, 52.9% yield) as a pale-yellow solid, which was taken to the next step without further purification. MS(ESI) m/z: 345.9 [M+H]⁺.

### Preparation of Intermediate 117: 7-chloro-8-fluoro-5-methoxy-2-(((4aS,7aR)-1-methyloctahydro-4aH-cyclopenta[b]pyridin-4a-yl)methoxy)-4-(2,2,2-trifluoroethoxy)pyrido[4,3-d]pyrimidine

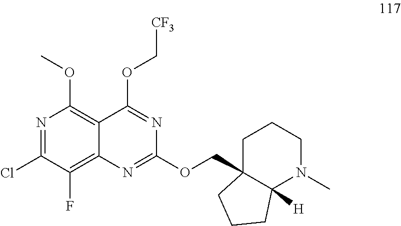 [SMILES: COc1nc(Cl)c(F)c2nc(OC[C@]34CCC[C@H]3N(C)CCC4)nc(OCC(F)(F)F)c12]

To a stirred solution of 2,7-dichloro-8-fluoro-5-methoxy-4-(2,2,2-trifluoroethoxy) pyrido[4,3-d] pyrimidine (Intermediate 116, 1.8 g, 2.60 mmol) in THE (20 mL) at room temperature, were added ((4aS,7aR)-1-methyloctahydro-4aH-cyclopenta[b]pyridin-4a-yl) methanol (0.44 g, 2.60 mmol), molecular sieves (250 mg) and DIPEA (1.36 mL, 7.80 mmol). The reaction mixture was stirred at room temperature over a period of 15 h. Then, the reaction mixture was filtered, evaporated under reduced pressure to get a crude residue, which was purified by silica gel column chromatography using COMBIFLASH instrument (40 g REDISEP™ column, 80 to 100% EtOAc-pet ether) to provide 7-chloro-8-fluoro-5-methoxy-2-(((4aS,7aR)-1-methyloctahydro-4aH-cyclopenta[b]pyridin-4a-yl)methoxy)-4-(2,2,2-trifluoroethoxy)pyrido[4,3-d]pyrimidine (400 mg, 0.83 mmol, 32.1% yield) as an off white solid. MS(ESI) m/z: 479.3 (M+H)⁺.

### Preparation of Intermediate 118: 8-fluoro-7-(7-fluoro-3-(methoxymethoxy)-8-((triisopropylsilyl)ethynyl)naphthalen-1-yl)-5-methoxy-2-(((4aS,7aR)-1-methyloctahydro-4aH-cyclopenta[b]pyridin-4a-yl)methoxy)-4-(2,2,2-trifluoroethoxy)pyrido[4,3-d]pyrimidine

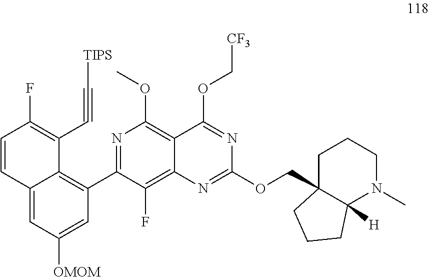

To a stirred solution of 7-chloro-8-fluoro-5-methoxy-2-(((4aS,7aR)-1-methyloctahydro-4aH-cyclopenta[b]pyridin-4a-yl)methoxy)-4-(2,2,2-trifluoroethoxy)pyrido[4,3-d]pyrimidine (Intermediate 117, 400 mg, 0.835 mmol) in THE (3.4 mL) at room temperature, were added ((2-fluoro-6-(methoxymethoxy)-8-(4,4,5,5-tetramethyl-1,3,2-dioxaborolan-2-yl)naphthalen-1-yl)ethynyl)triisopropylsilane (428 mg, 0.83 mmol) and 1.5M potassium phosphate tribasic (1.67 mL, 2.51 mmol) aqueous solution. The reaction mixture was purged with argon and charged with methanesulfonato(diadamantyl-n-butylphosphino)-2′-amino-1,1′-biphenyl-2-yl)palladium(II) dichloromethane adduct, min. 95% [cataCXium® A Palladacycle Gen. 3] (60.80 mg, 0.08 mmol). The resulting solution was purged with argon for additional 3 min and was heated at 65° C. for 4 h. The reaction mixture was diluted with water and extracted with ethyl acetate. The combined organic layer was washed with brine, dried over anhydrous sodium sulfate, concentrated under reduced pressure to get a crude residue, which was purified by silica gel column chromatography using CombiFlash instrument (12 g REDISEP™ column, 30-80% EtOAc/Pet-ether) to provide 8-fluoro-7-(7-fluoro-3-(methoxymethoxy)-8-((triisopropylsilyl)ethynyl)-naphthalen-1-yl)-5-methoxy-2-(((4aS,7aR)-1-methyloctahydro-4aH-cyclopenta[b]pyridin-4a-yl)methoxy)-4-(2,2,2-trifluoroethoxy)pyrido[4,3-d]pyrimidine (550 mg, 0.66 mmol, 79% yield) as a brown solid. MS(ESI) m/z: 829.3 (M+H)⁺.

### Preparation of Intermediate 119: 8-fluoro-7-(7-fluoro-3-(methoxymethoxy)-8-((triisopropylsilyl)ethynyl)naphthalen-1-yl)-5-methoxy-2-(((4aS,7aR)-1-methyloctahydro-4aH-cyclopenta[b]pyridin-4a-yl)methoxy)-4-(2,2,2-trifluoroethoxy)pyrido[4,3-d]pyrimidine

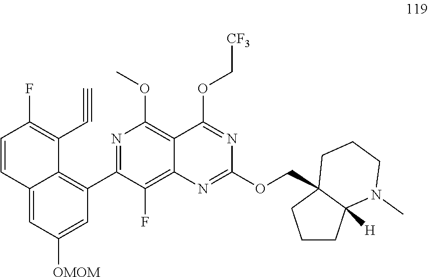

To a stirred solution of 8-fluoro-7-(7-fluoro-3-(methoxymethoxy)-8-((triisopropylsilyl)ethynyl)naphthalen-1-yl)-5-methoxy-2-(((4aS,7aR)-1-methyloctahydro-4aH-cyclopenta[b]pyridin-4a-yl)methoxy)-4-(2,2,2-trifluoroethoxy)pyrido[4,3-d]pyrimidine (Intermediate 118, 550 mg, 0.66 mmol) in DMF (2 mL) was added cesium fluoride (1.01 g, 6.63 mmol) and the reaction mixture was stirred at room temperature for 1 h. The reaction mixture was diluted with water, and extracted with EtOAc. The organic layer was washed with brine, dried over anhydrous sodium sulfate, filtered, and concentrated under reduced pressure to get crude 7-(8-ethynyl-7-fluoro-3-(methoxymethoxy)naphthalen-1-yl)-8-fluoro-5-methoxy-2-(((4aS,7aR)-1-methyloctahydro-4aH-cyclopenta[b]pyridin-4a-yl)methoxy)-4-(2,2,2-trifluoroethoxy)pyrido[4,3-d]pyrimidine (400 mg, 0.59 mmol, 90% yield)) as a brown oil, which was taken to the next step without further purification. MS(ESI) m/z: 673.2 [M+H]⁺.

### Preparation of Intermediate 120: 7-(8-ethynyl-7-fluoro-3-(methoxymethoxy)naphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-ol

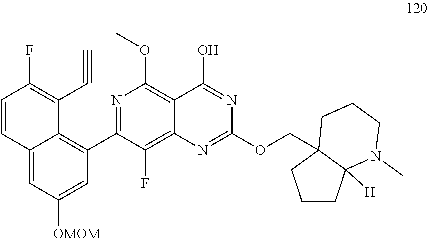

To a stirred solution of 7-(8-ethynyl-7-fluoro-3-(methoxymethoxy)naphthalen-1-yl)-8-fluoro-5-methoxy-2-(((4aS,7aR)-1-methyloctahydro-4aH-cyclopenta[b]pyridin-4a-yl)methoxy)-4-(2,2,2-trifluoroethoxy)pyrido[4,3-d]pyrimidine (Intermediate 119, 400 mg, 0.595 mmol) in THE (3 mL)-EtOH (3 mL) at room temperature was was added 1M aqueous NaOH solution (2.97 mL, 2.97 mmol) and the resulting reaction mixture was stirred at room temperature for 15 h. The volatiles were removed under reduced pressure and the crude residue was dissolved in ethyl acetate (50 ml). The organic layer was washed with water, brine, dried over anhydrous sodium sulfate, filtered, and concentrated under reduced pressure to provide crude 7-(8-ethynyl-7-fluoro-3-(methoxymethoxy)naphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-ol (300 mg, 0.508 mmol, 85% yield) as an off white solid, which was used in the next step without further purification. MS(ESI) m/z: 591.2 [M+H]⁺.

### Preparation of Intermediate 121: (S)-4-(7-(8-ethynyl-7-fluoro-3-(methoxymethoxy)naphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-yl)-6-methyl-1,4-oxazepan-6-ol

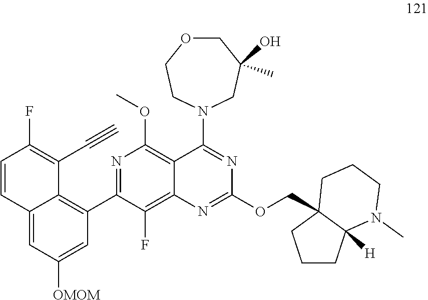

To a stirred solution of 7-(8-ethynyl-7-fluoro-3-(methoxymethoxy)naphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-ol (Intermediate 120, 300 mg, 0.51 mmol) in acetonitrile (10 mL) at room temperature, were added (S)-6-methyl-1,4-oxazepan-6-ol hydrochloride (128 mg, 0.76 mmol), PyBOP (396 mg, 0.76 mmol) and DIPEA (0.27 mL, 1.52 mmol). The reaction mixture was heated at 80° C. for 8 h. The reaction mixture was cooled to room temperature, concentrated under reduced pressure to get a crude residue, which was diluted with ethyl acetate. The organic layer was washed with water, brine, dried over anhydrous sodium sulfate, filtered and concentrated under reduced pressure to get crude (S)-4-(7-(8-ethynyl-7-fluoro-3-(methoxymethoxy)naphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-yl)-6-methyl-1,4-oxazepan-6-ol (250 mg, 0.35 mmol, 69.9% yield) as a brown solid, which was used in the next step without further purification. MS(ESI) m/z: 704.3 [M+H]⁺.

### Example 12-1

### 4-(2-{[(4aS,7aR)-1-methyl-octahydro-1H-cyclopenta[b]pyridin-4a-yl]methoxy}-7-(8-ethyl-7-fluoro-3-hydroxynaphthalen-1-yl)-8-fluoropyrido[4,3-d]pyrimidin-4-yl)-6-methyl-1,4-oxazepan-6-ol

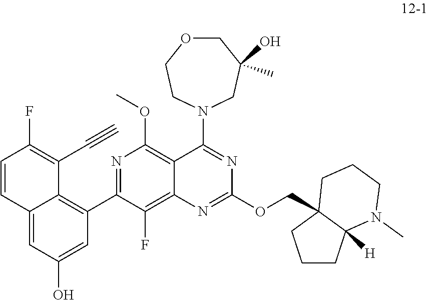 [SMILES: C#Cc1c(F)ccc2cc(O)cc(-c3nc(OC)c4c(N5CCOC[C@@](C)(O)C5)nc(OC[C@]56CCC[C@H]5N(C)CCC6)nc4c3F)c12]

To a stirred solution of (S)-4-(7-(8-ethynyl-7-fluoro-3-(methoxymethoxy)naphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-yl)-6-methyl-1,4-oxazepan-6-ol (Intermediate 121, 250 mg, 0.35 mmol) in ethyl acetate (5 mL) at 0° C., was added 1M HCl in ethyl acetate (5.33 mL, 5.33 mmol) and the reaction mixture was stirred for 1 h. Then, the volatiles were removed under reduced pressure (at lower temperature, ˜30° C.) and the crude residue was co-evaporated with 1,4-dioxane. The crude residue was re-dissolved in 1,4-dioxane and neutralized with trimethylamine. The reaction mixture was concentrated under reduced pressure to get a crude residue, which was purified by Prep-HPLC [HPLC Method: Preparative column: Gemini NX C18 (250*21.2*5); Mobile Phase A:10 mm ammonium bicarbonate in water pH-9.5; Mobile Phase B: ACN:MeOH (1:1) Flow:20 ml\min; temperature: 27° C.; Flow rate: 19.0 mL/min; detection: UV at 220 nm.] to provide (S)-4-(7-(8-ethynyl-7-fluoro-3-hydroxynaphthalen-1-yl)-8-fluoro-5-methoxy-2-(((4aS,7aR)-1-methyloctahydro-4aH-cyclopenta[b]pyridin-4a-yl)methoxy)pyrido[4,3-d]pyrimidin-4-yl)-6-methyl-1,4-oxazepan-6-ol (76 mg, 0.11 mmol, 31.8% yield) as an off-white solid. MS(ESI) m/z: 660.3 [M+H]⁺; ¹H NMR (400 MHz, DMSO-d₆) δ ppm=9.96 (br s, 1H), 7.95 (dd, J=9.1, 6.0 Hz, 1H), 7.44 (t, J=8.9 Hz, 1H), 7.36 (d, J=2.4 Hz, 1H), 7.23 (t, J=2.8 Hz, 1H), 5.31-4.95 (m, 1H), 4.44 (d, J=10.6 Hz, 1H), 4.19-4.02 (m, 3H), 3.99-3.82 (m, 5H), 3.78-3.65 (m, 2H), 3.61-3.49 (m, 3H), 2.66-2.57 (m, 1H), 2.56-2.52 (m, 1H), 2.32-2.14 (m, 4H), 1.86-1.78 (m, 2H), 1.77-1.49 (m, 7H), 1.46-1.31 (m, 1H), 1.14-0.99 (m, 3H).

### BIOLOGICAL ACTIVITY

### KRAS^{G12D} RAF Disruption Assay

Recombinant GMPPNP-loaded KRAS G12D (5 nM) was treated with compound at room temperature for 20 minutes in assay buffer (50 mM Tris pH 7.5, 100 mM NaCl, 1 mM MgCl2, 1 mM dithiothreitol (DTT), 100 μg/ml bovine serum albumin (BSA)). Recombinant GST-RAF1 RBD (Ras binding domain) (9 nM) was added, followed by the addition of SA-Tb (0.25 nM), and the reaction mixture was incubated for 3 hours. HTRF (Homogeneous Time-Resolved Fluorescence) signal was measured (PerkinElmer Envision), the signal ratio (λₑₘ 520/λₑₘ 495) was calculated, and IC₅₀ values were calculated from the dose-response curve.

### KRAS^{G12D} Nucleotide Exchange Assay

Recombinant GDP-loaded KRAS G12D (20 nM) was treated with compound at room temperature for 20 minutes in assay buffer (10 mM Hepes (N-(2-hydroxyethyl)piperazine-N-(2-ethanesulfonic acid)) pH 7.4, 150 mM NaCl, 5 mM MgCl2, 0.0025% IGEPAL™-CA630 (Stepan, Northbrook, IL) (octylphenoxypolyethoxyethanol), 0.05% BSA, 1 mM DTT, 0.5 nM SA-Tb). BIODIPY-labeled GDP (BIODIPY: dipyrrometheneboron difluoride) (400 nM) and recombinant SOS (son of sevenless protein) (10 nM) were added, and the reaction was incubated for 30 minutes. HTRF signal was measured (PerkinElmer Envision), the signal ratio (λₑₘ 520/λₑₘ 495) was calculated, and IC₅₀ values were calculated from the dose-response curve.

The IC₅₀ values for compounds described herein are shown in Table 9.

TABLE 9

| | KRASG12D RAF | KRASG12D Nucleotide |
|---|---|---|
| | Disruption Assay | Exchange Assay |
| Example # | (IC₅₀, μM) | (IC₅₀, μM) |
| 1-1 | 0.42 | 0.01 |
| 1-2 | 0.01 | 0.005 |
| 1-3 | <0.002 | <0.002 |
| 1-4 | 2.77 | 0.02 |
| 1-5 | 8.42 | 0.14 |
| 1-6 | 7.09 | 0.05 |
| 1-7 | 0.35 | 0.01 |
| 1-8 | 2.82 | 0.07 |
| 1-9 | 0.25 | 0.01 |
| 1-10 | 3.64 | 0.18 |
| 1-11 | 1.20 | 0.06 |
| 1-12 | 1.0 | <0.002 |
| 1-13 | 2.02 | 0.03 |
| 1-14 | 20.63 | 0.20 |
| 1-15 | 8.42 | 0.14 |
| 1-16 | 8.87 | 0.42 |
| 1-17 | 6.57 | 0.08 |
| 1-18 | 70.71 | 1.12 |
| 1-19 | 10.88 | 0.06 |
| 1-20 | 3.64 | 0.18 |
| 1-21 | 10.39 | 0.11 |
| 1-22 | 0.62 | 0.002 |
| 1-23 | 0.002 | 0.002 |
| 1-24 | 0.67 | 0.002 |
| 1-25 | 0.68 | 0.005 |
| 1-26 | 0.29 | 0.01 |
| 1-27 | 3.82 | 0.04 |
| 1-28 | 3.41 | 0.06 |
| 1-29 | 21.84 | 0.13 |
| 1-30 | 6.69 | 0.22 |
| 1-31 | 8.61 | 0.18 |
| 1-32 | 64.81 | 1.67 |
| 1-33 | 54.70 | 0.90 |
| 1-34 | 0.81 | 0.02 |
| 1-35 | 0.39 | 0.02 |
| 1-36 | 0.81 | 0.09 |
| 1-37 | 0.12 | 0.01 |
| 1-38 | 0.32 | 0.01 |
| 1-39 | 0.35 | 0.03 |
| 1-40 | 1.26 | 0.03 |
| 1-41 | 17.41 | 0.11 |
| 1-42 | 1.25 | 0.07 |
| 1-43 | 0.93 | 0.05 |
| 1-44 | >100 | 19.10 |
| 1-45 | 9.80 | 0.35 |
| 1-46 | 0.73 | 0.09 |
| 1-47 | 0.30 | 0.03 |
| 1-48 | 2.62 | 0.02 |
| 1-49 | 0.63 | 0.04 |
| 1-50 | 0.28 | 0.003 |
| 1-51 | 3.7 | 0.16 |
| 1-52 | 0.05 | 0.003 |
| 1-53 | 0.93 | 0.02 |
| 1-54 | 0.71 | 0.06 |
| 1-55 | 11.2 | 1.79 |
| 1-56 | 4.27 | 0.28 |
| 1-57 | 2.96 | 0.07 |
| 1-58 | 0.06 | 0.003 |
| 1-59 | 1.29 | 0.03 |
| 1-60 | 0.11 | 0.03 |
| 1-61 | 1.39 | 0.05 |
| 1-62 | 50.15 | 2.08 |
| 1-63 | 100.00 | 12.83 |
| 1-64 | 3.79 | 0.06 |
| 1-65 | 4.87 | 0.08 |
| 1-66 | 43.00 | 17.4 |
| 1-67 | 5.60 | 0.26 |
| 1-68 | 6.66 | 0.03 |
| 1-69 | 0.62 | 0.05 |
| 1-70 | 10.81 | 0.08 |
| 1-71 | 5.42 | 0.03 |
| 1-72 | 39.60 | 0.55 |
| 1-73 | 12.72 | 0.11 |
| 1-74 | 16.45 | 0.05 |
| 1-75 | 0.32 | 0.01 |
| 1-76 | 0.91 | 0.04 |
| 1-77 | 0.70 | 0.01 |
| 1-78 | 0.44 | 0.03 |
| 1-79 | 0.07 | 0.02 |
| 1-80 | 0.02 | 0.01 |
| 1-81 | 0.03 | 0.009 |
| 1-82 | 0.26 | 0.02 |
| 1-83 | 0.11 | 0.02 |
| 1-84 | 0.06 | 0.009 |
| 1-85 | 0.04 | 0.01 |
| 1-86 | 0.52 | 0.17 |
| 1-87 | 0.07 | 0.007 |
| 1-88 | 0.003 | 0.005 |
| 1-89 | 0.005 | 0.006 |
| 1-90 | 0.03 | 0.02 |
| 1-91 | 0.10 | 0.04 |
| 1-92 | 0.290 | 0.008 |
| 1-93 | 0.190 | 0.010 |
| 1-94 | 0.30 | 0.010 |
| 1-95 | 0.02 | 0.06 |
| 1-96 | 0.39 | 0.02 |
| 1-98 | 0.48 | 0.007 |
| 1-99 | 0.15 | 0.007 |
| 1-100 | 0.09 | 0.006 |
| 1-101 | 0.28 | 0.24 |
| 1-102 | 0.40 | 0.02 |
| 1-103 | 1.0 | 0.49 |
| 1-104 | 0.06 | 0.01 |
| 1-105 | 4.9 | 0.05 |
| 1-106 | 0.01 | 0.01 |
| 1-107 | 0.19 | 0.05 |
| 1-108 | 0.007 | 0.004 |
| 1-109 | 0.80 | 0.01 |
| 1-110 | 0.04 | 0.006 |
| 1-111 | 0.12 | 0.006 |
| 1-112 | 0.13 | 0.033 |
| 1-113 | 0.60 | 0.057 |
| 1-114 | 0.02 | 0.013 |
| 1-115 | 0.02 | 0.002 |
| 1-116 | 0.40 | 0.016 |
| 1-117 | 0.12 | 0.006 |
| 1-118 | 0.04 | 0.005 |
| 1-119 | 0.06 | 0.010 |
| 1-120 | 0.004 | 0.006 |
| 1-121 | 0.04 | 0.007 |
| 1-122 | 0.006 | 0.002 |
| 1-123 | 0.20 | 0.004 |
| 1-124 | 0.28 | 0.010 |
| 1-125 | 0.009 | 0.003 |
| 1-126 | 0.09 | 0.007 |
| 1-127 | 0.20 | 0.002 |
| 1-128 | 0.02 | 0.01 |
| 1-129 | 0.08 | 0.008 |
| 1-130 | 0.002 | 0.002 |
| 1-131 | 0.070 | 0.003 |
| 1-132 | 0.003 | 0.002 |
| 1-133 | 0.16 | 0.006 |
| 1-134 | 0.03 | 0.006 |
| 1-135 | 0.04 | 0.02 |
| 1-136 | 0.15 | 0.007 |
| 1-137 | 0.29 | 0.008 |
| 1-138 | 0.47 | 0.01 |
| 1-139 | 0.06 | 0.01 |
| 1-140 | 0.19 | 0.01 |
| 1-141 | 0.48 | 0.007 |
| 1-142 | 0.56 | 0.01 |
| 1-143 | 0.24 | 0.03 |
| 1-144 | 0.40 | 0.02 |
| 1-145 | 0.76 | 0.008 |
| 1-146 | 0.51 | 0.02 |
| 1-147 | 0.11 | 0.01 |
| 1-148 | 0.32 | 0.01 |
| 1-149 | 20.4 | 0.04 |
| 1-150^{C} | 18.2 | 1.20 |
| 1-151 ^{C} | 67.5 | 13.3 |
| 1-152 | 14.2 | 0.16 |
| 1-153^{B} | 0.29 | 0.02 |
| 1-154^{B} | 0.005 | 0.00 |
| 1-155 | 0.22 | 0.01 |
| 1-156 | 0.13 | 0.03 |
| 1-157 | 8.18 | 0.07 |
| 1-158 | 0.33 | 0.03 |
| 2-1 | 0.72 | 0.02 |
| 2-2 | 0.03 | 0.004 |
| 2-3 | 0.12 | 0.007 |
| 2-4 | 0.83 | 0.03 |
| 2-5 | 1.66 | 0.08 |
| 2-6 | 0.92 | 0.07 |
| 2-7 | 1.61 | 0.12 |
| 2-8 | 0.23 | 0.008 |
| 2-9 | 1.31 | 0.05 |
| 2-10 | 33.33 | 0.005 |
| 2-11 | 2.62 | 2.61 |
| 2-12 | 3.33 | 0.12 |
| 2-13 | 0.29 | 0.02 |
| 2-14 | 4.62 | 1.27 |
| 2-15 | 1.65 | 0.08 |
| 2-16 | 15.96 | 0.53 |
| 2-17 | 27.02 | 0.29 |
| 2-18 | 35.67 | 0.10 |
| 2-19 | >100 | 2.39 |
| 2-20 | 14.25 | 0.76 |
| 2-21 | 29.17 | 3.86 |
| 2-22 | 4.77 | 0.03 |
| 2-23 | 1.17 | 0.10 |
| 2-24 | 9.50 | 0.08 |
| 2-25 | 0.98 | 0.002 |
| 2-26 | 4.92 | 0.080 |
| 2-27 | 0.70 | 0.01 |
| 2-28 | 0.20 | 0.01 |
| 2-29 | 1.02 | 0.02 |
| 2-30 | 0.66 | 0.003 |
| 2-31 | 0.81 | 0.09 |
| 2-32 | 0.06 | 0.03 |
| 2-33 | 6.94 | 0.19 |
| 2-34 | 0.42 | 0.03 |
| 2-35 | 1.35 | 0.10 |
| 2-36 | 0.83 | 0.02 |
| 2-37 | 0.17 | 0.01 |
| 2-38 | 0.95 | 0.07 |
| 2-39 | 3.83 | 0.12 |
| 2-40 | 0.11 | 0.02 |
| 2-41 | 0.68 | 0.02 |
| 2-42 | 0.96 | 0.04 |
| 2-43 | 4.10 | 0.20 |
| 2-44 | 0.72 | 0.02 |
| 2-45 | 7.14 | 0.56 |
| 2-46 | 2.60 | 0.11 |
| 2-47 | 1.20 | 0.07 |
| 2-48 | 0.82 | 0.06 |
| 2-49 | 0.89 | 0.05 |
| 2-50 | 11.59 | 9.77 |
| 2-51 | 7.82 | 0.14 |
| 2-52 | 14.86 | 0.59 |
| 2-53 | 11.32 | 10.29 |
| 2-54 | 6.62 | 0.93 |
| 2-55 | 5.34 | 0.20 |
| 2-57 | 0.70 | 0.06 |
| 2-58 | 0.09 | 0.01 |
| 2-59 | 0.32 | 0.03 |
| 2-60 | 0.31 | 0.06 |
| 2-61 | 0.21 | 0.01 |
| 2-63 | 0.10 | 0.03 |
| 2-64 | 0.09 | 0.01 |
| 2-65 | 0.03 | 0.01 |
| 2-66 | 0.09 | 0.01 |
| 2-67 | 2.70 | 0.16 |
| 2-68 | 0.10 | 0.04 |
| 2-69 | 1.80 | 0.04 |
| 2-70 | 0.02 | 0.007 |
| 2-71 | 0.24 | 0.003 |
| 2-72 | 0.02 | 0.008 |
| 2-73 | 1.20 | 0.87 |
| 2-74 | 0.04 | 0.01 |
| 2-75 | 0.18 | 0.02 |
| 2-76 | 0.11 | 0.01 |
| 2-77 | 0.57 | 0.02 |
| 2-78 | 0.04 | 0.005 |
| 2-79 | 0.91 | 0.04 |
| 2-80 | 1.39 | 0.60 |
| 2-81 | 0.11 | 0.008 |
| 2-82 | 0.15 | 0.07 |
| 2-83 | 1.04 | 0.03 |
| 3-1 | 54.40 | 2.60 |
| 4-1 | 7.50 | 0.27 |
| 5-1 | 0.003 | 0.006 |
| 6-1 | 2.50 | 0.32 |
| 6-2 | 0.90 | <0.002 |
| 6-3 | 0.90 | 0.005 |
| 6-4 | 1.20 | 0.006 |
| 6-5 | 3.50 | 0.02 |
| 6-6 | 3.80 | 0.003 |
| 6-7 | 3.40 | 0.09 |
| 6-8 | 4.0 | 0.03 |
| 6-9 | 0.67 | 0.02 |
| 6-10 | 5.60 | 0.01 |
| 6-11 | 0.03 | 0.01 |
| 7-1 | 15.4 | 0.08 |
| 7-2 | 1.10 | 0.004 |
| 7-3 | 0.08 | 0.05 |
| 7-4 | 6.60 | <0.002 |
| 7-5 | 1.70 | 0.02 |
| 7-6 | 5.80 | 0.03 |
| 7-7 | 0.007 | <0.002 |
| 7-8 | 1.80 | 0.009 |
| 7-9 | 1.70 | 0.02 |
| 8-1 | 51.7 | 1.10 |
| 8-2 | 13.6 | 1.0 |
| 9-1 | 0.009 | 0.009 |
| 9-2 | 0.030 | 0.006 |
| 9-3 | 0.33 | 0.005 |
| 9-4 | 0.02 | 0.009 |
| 9-5 | 34.1 | 8.84 |
| 9-6 | 0.26 | 0.006 |
| 9-7 | 0.24 | 0.003 |
| 9-8 | 0.04 | 0.004 |
| 9-9 | 0.02 | 0.002 |
| 9-10 | 0.64 | 0.007 |
| 9-11 | 0.07 | 0.005 |
| 9-12 | 1.3 | 0.008 |
| 9-13 | 0.10 | 0.02 |
| 9-14 | 0.16 | 0.03 |
| 9-15 | 0.03 | 0.005 |
| 9-16 | 0.08 | 0.006 |
| 9-17 | 0.04 | 0.002 |
| 9-18 | 1.03 | 0.03 |
| 9-19 | 0.007 | 0.002 |
| 9-20 | 0.20 | 0.018 |
| 9-21 | 2.15 | 0.12 |
| 9-22 | 0.33 | 0.02 |
| 9-23 | 0.07 | 0.007 |
| 9-24 | 0.11 | 0.008 |
| 9-25 | 0.34 | 0.01 |
| 9-26 | 0.009 | 0.006 |
| 9-27 | 0.02 | 0.008 |
| 928 | 0.08 | 0.006 |
| 9-29 | 0.19 | 0.007 |
| 9-30 | 0.02 | 0.01 |
| 9-31 | 0.55 | 0.01 |
| 9-32 | 0.16 | 0.006 |
| 9-33 | 0.23 | 0.006 |
| 9-34 | 0.27 | 0.009 |
| 9-35 | 11.2 | 0.68 |
| 9-36 | 0.12 | 0.008 |
| 9-37 | 1.06 | 0.17 |
| 9-38 | 0.03 | 0.005 |
| 9-39 | 0.20 | 0.01 |
| 9-40 | 0.03 | 0.006 |
| 9-41 | 0.06 | 0.008 |
| 9-42 | 1.48 | 0.008 |
| 10-1 | 0.41 | 0.01 |
| 10-2 | 9.21 | 0.04 |
| 10-3 | 1.06 | <0.002 |
| 10-4 | 0.83 | 0.02 |
| 10-5 | 21.8 | 29.3 |
| 10-6 | >100 | >100 |
| 10-7 | >100 | >100 |
| 10-8 | >100 | >100 |
| 10-9 | 49.8 | 19.5 |
| 11-1 | 0.783 | 0.011 |
| 11-2 | 9.980 | 0.074 |
| 11-3 | 3.754 | 0.007 |
| 12-1 | 0.01 | 0.01 |

### Cellular KRAS:RAF Disruption Assay

Stable cell lines (HEK293 T) were generated to co-express RAF1 and mutant or wild type KRAS, as indicated, using the BiBRET vector (Promega). Engineered cell lines were suspended in media (OptiMEM, 400 FBS), plated in 384-well plates (8000 cells/well), and allowed to rest for 1.5 hours. Cells were treated with compounds for 24 hours, and NanoBRET NanoGlo substrate (Promega) was added per the manufacturer's instructions. NanoBRET signal was measured (PerkinElmer Envision), the signal ratio (λₑₘ 618/λ_{ecm} 460) was calculated, and IC50 values were calculated from the dose-response curve.

TABLE 10

| | RAS::RAF binding via NanoBRET IC50, μM | | | | | | | | | |
|---|---|---|---|---|---|---|---|---|---|---|
| Ex. | KRAS | KRAS | KRAS | KRAS | KRAS | KRAS | KRAS | KRAS | KRAS | KRAS |
| Number | G12A | G12C | G12V | G13D | WT | G12R | G12S | Q61H | Q61R | A146T |
| 1-1 | 0.016 | 0.025 | 0.199 | 0.045 | 0.006 | 5.304 | 0.029 | 0.099 | >10 | 0.076 |
| 1-2 | 0.0006 | 0.002 | 0.007 | 0.002 | 0.0004 | 0.048 | 0.001 | 0.004 | 0.713 | 0.006 |
| 1-3 | 0.002 | 0.003 | 0.019 | 0.005 | 0.002 | 0.133 | 0.004 | 0.007 | 1.461 | 0.016 |
| 1-35 | 0.01 | 0.012 | 0.032 | 0.022 | 0.013 | 4.231 | 0.011 | 0.031 | >10 | 0.04 |
| 1-37 | 0.0105 | 0.0166 | 0.0545 | | | | | | | |
| 1-89 | 0.0048 | 0.0093 | 0.0201 | | 0.0085 | | | | | |
| 1-90 | 0.0195 | 0.0416 | 0.1971 | | 0.0119 | | | | | |
| 1-99 | 0.012 | 0.011 | 0.126 | | 0.003 | | | | | |
| 1-100 | 0.0046 | 0.0129 | 0.1182 | | 0.0123 | | | | | |
| 1-107 | 0.0053 | 0.0098 | 0.1331 | | 0.0097 | | | | | |
| 1-108 | 0.0132 | 0.0188 | 0.1404 | | 0.0172 | | | | | |
| 2-2 | 0.003 | 0.005 | 0.026 | 0.008 | 0.002 | 0.204 | 0.006 | 0.016 | 2.293 | 0.017 |
| 2-3 | 0.018 | 0.024 | 0.172 | 0.049 | 0.011 | 1.075 | 0.039 | 0.072 | >5 | 0.081 |
| 2-72 | 0.0090 | 0.0128 | 0.0405 | | 0.0036 | | | | | |
| 5-1 | 0.881 | 0.826 | 2.513 | | 0.238 | | | | | |
| 6-2 | 0.029 | 0.117 | 0.405 | 0.072 | 0.069 | >10 | 0.109 | 0.234 | >10 | 0.179 |
| 7-3 | 0.144 | 0.367 | 0.68 | 0.199 | 0.04 | 7.509 | 0.313 | 0.474 | >10 | 0.315 |
| 7-7 | 0.226 | 0.346 | >5 | 0.407 | 0.242 | >5 | 0.422 | 0.504 | >5 | 0.577 |

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. A compound of formula (I): 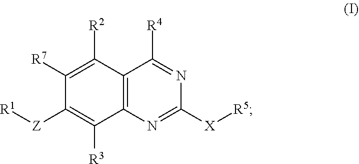
or a pharmaceutically acceptable salt thereof, wherein:
Z is a bond, O, NR^{e} or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from the group consisting of C₂-C₄alkenyl, C₁-C₃alkoxy, C₁-C₃alkyl, C₂-C₄alkynyl, C₂-C₄alkynyloxy, amino, aminoC₁-C₃alkyl, cyano, cyanoC₁-C₃alkoxy, C₃-C₈cycloalkyl optionally substituted with one, two, or three halo groups, halo, haloC₁-C₃alkyl, haloC₁-C₃alkoxy, hydroxy, hydroxyC₁-C₃alkyl, heteroaryl, heterocyclyl, and phenyl, wherein the heteroaryl, heterocyclyl, and phenyl are optionally substituted with one, two, or three substituents independently selected from the group consisting of C₁-C₃alkoxy, C₁-C₃alkyl, halo, and haloC₁-C₃alkyl;
R², R³, and R⁷ are independently selected from the group consisting of hydrogen, C₁-C₃alkoxy, C₁-C₃alkyl, cyano, halo, haloC₁-C₃alkyl, —C(O)NH₂, —C(O)NH(C₁-C₃alkyl), —C(O)N(C₁-C₃alkyl)₂, and hydroxy;
R⁴ is —NHR⁵⁰ or 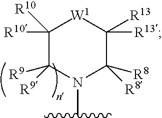
wherein
R⁵⁰ is a five membered ring optionally containing one or two heteroatoms independently selected from the group consisting of nitrogen and oxygen, wherein the ring is optionally substituted with one or two groups independently selected from C₁-C₃alkyl and oxo;
n′ is 0, 1, 2, or 3;
R⁸, R^{8′}, R⁹, R^{9′}, R¹⁰, R^{10′}, R¹³, and R^{13′} are each independently selected from the group consisting of hydrogen, C₁-C₃alkoxy, C₁-C₆alkoxyC₁-C₆alkyl, C₁-C₃alkyl, C₃-C₆cycloalkyl, cyano, halo, haloC₁-C₃alkyl, hydroxy, and hydroxyC₁-C₃alkyl; or
R⁸ and R⁹, together form a C₁-C₃alkylene; or
R⁸ and R¹⁰ together form C₁-C₃alkylene; or
R⁸ and R¹³ together form C₁-C₃alkylene; or
R⁹ and R¹³ together form C₁-C₃alkylene; or
R¹⁰ and R¹³ together form C₁-C₃alkylene; or
R⁸ and R^{8′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R⁹ and R^{9′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R¹⁰ and R^{10′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R¹³ and R^{13′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy;
W¹ is CR¹¹R¹², NR¹⁷, NR^{15″}C(O), N(C(O)(CH₂)ₙOR¹⁵), O, SO₂, SO₂NR^{15′}, or P(O)CH₃; wherein
n is 0 or 1;
R¹¹ and R¹² are independently selected from the group consisting of hydrogen, C₁-C₃alkoxy, C₁-C₆alkoxyC₁-C₆alkyl, C₁-C₃alkyl, cyano, dimethylphosphino; dimethylsulfonamide, halo, hydroxy, and methylsulfonyl; or
R¹¹ and R¹², together with the atoms to which they are attached, form a four- to six-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R¹¹ and R¹³, together with the atoms to which they are attached, form a three-, four- or five-membered ring optionally containing one or two heteroatoms independently selected from nitrogen and oxygen, wherein the ring optionally contains one or two double bonds, and wherein the ring is optionally substituted with two groups selected from the group consisting of C₁-C₃alkyl, halo, haloC₁-C₃alkyl, and hydroxy; or
R¹³ and R¹⁵, together form CH₂;
R_{15′} is C₁-C₃alkyl or C₁-C₆alkoxyC₁-C₆alkyl;
R^{15″} is hydrogen or C₁-C₃alkyl;
R¹⁷ is selected from the group consisting of C₁-C₃alkylcarbonyl, C₃-C₆cycloalkylcarbonyl, haloC₁-C₃alkylcarbonyl, methylsulfonyl, and tetrahydropyranylcarbonyl, wherein the C₃-C₆cycloalkyl and the tetrahydropyranyl are optionally substituted with one or two substituents independently selected from the group consisting of cyano, halo, and hydroxy;
X is O or NR¹⁶, wherein R¹⁶ is hydrogen or C₁-C₃alkyl;
R⁵ is selected from the group consisting of hydrogen, C₁-C₆alkoxyC₁-C₆alkyl, C₁-C₆alkyl, aryl, arylC₁-C₆alkyl, carboxyC₁-C₆alkyl, C₃-C₆cycloalkyl, C₃-C₆cycloalkylC₁-C₆alkyl, di(C₁-C₃alkyl)aminoC₂-C₆alkyl, haloC₁-C₆alkyl, heteroaryl, heteroarylC₁-C₆alkyl, heterocyclyl, heterocyclylC₁-C₆alkyl, hydroxyC₁-C₆alkyl, NR^{a}R^{b}—C(O)—C₁-C₆alkyl), NR^{a}R^{b}C₁-C₆alkyl, wherein the aryl, the aryl part of the arylC₁-C₆alkyl, the C₃-C₆cycloalkyl, the cycloalkyl part of the C₃-C₆cycloalkylC₁-C₆alkyl, the heteroaryl, the heteroaryl part of the heteroarylC₁-C₆alkyl, the heterocyclyl, the heterocyclyl part of the heterocyclylC₁-C₆alkyl, are optionally substituted with one, two, three, or four groups independently selected from C₁-C₃alkoxy, C₁-C₃alkyl, deuterated C₁-C₃alkyl, C₃-C₆cycloalkyl, (C₁-C₆alkyl)amino, (C₁-C₆alkyl)aminoC₁-C₃alkyl, amino, aminoC₁-C₃alkyl, carboxy, cyano, di(C₁-C₆alkyl)amino, di(C₁-C₆alkyl)aminoC₁-C₃alkyl, halo, haloC₁-C₃alkoxy, haloC₁-C₃alkyl, heterocyclyl, heterocyclylC₁-C₃alkyl, hydroxy, hydroxyC₁-C₃alkyl, nitro, and oxo; wherein the heterocyclyl and the heterocyclyl part of the heterocyclylC₁-C₃alkyl is further optionally substituted with one, two, or three groups independently selected from the group consisting of C₁-C₃alkoxy, C₁-C₃alkyl, halo, and haloC₁-C₃alkyl; or
R⁵ and R¹⁶, together with the nitrogen atom to which they are attached, form a heterocyclic group optionally substituted with one, two, three, four, or five groups independently selected from the group consisting of one, two, three, or four groups independently selected from the group consisting of C₁-C₃alkoxy, C₁-C₃alkoxyalkyl, C₁-C₃alkyl, amino, aminoC₁-C₃alkyl, hydroxy, and hydroxyC₁-C₃alkyl; and
one of R^{a} and R^{b} is selected from the group consisting of hydrogen and C₁-C₃alkyl and the other is selected from the group consisting of hydrogen, C₁-C₃alkyl, C₁-C₃alkoxycarbonyl, C₁-C₃alkylcarbonyl, arylC₁-C₆alkyl, C₃-C₆cycloalkyl, and C₃-C₆cycloalkylC₁-C₆alkyl.

2. (canceled)

3. (canceled)

4. A compound of formula (II): 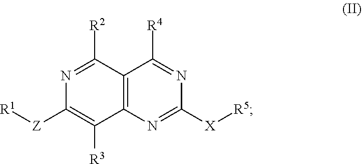
or a pharmaceutically acceptable salt thereof, wherein:
Z is a bond, O, NR^{e} or CR^{e}R^{f}, wherein R^{e} and R^{f} are independently hydrogen or C₁-C₃alkyl;
R¹ is aryl or heteroaryl, wherein the aryl and the heteroaryl are optionally substituted with one, two, three, four, or five substituents independently selected from the group consisting of C₂-C₄alkenyl, C₁-C₃alkoxy, C₁-C₃alkyl, C₂-C₄alkynyl, C₂-C₄alkynyloxy, amino, aminoC₁-C₃alkyl, cyano, cyanoC₁-C₃alkoxy, C₃-C₈cycloalkyl optionally substituted with one, two, or three halo groups, halo, haloC₁-C₃alkyl, haloC₁-C₃alkoxy, hydroxy, hydroxyC₁-C₃alkyl, heteroaryl, heterocyclyl, and phenyl, wherein the heteroaryl, heterocyclyl, and phenyl are optionally substituted with one, two, or three substituents independently selected from the group consisting of C₁-C₃alkoxy, C₁-C₃alkyl, halo, and haloC₁-C₃alkyl;
R² and R³ are independently selected from the group consisting of hydrogen, C₁-C₃alkoxy, C₁-C₃alkyl, cyano, halo, haloC₁-C₃alkyl, —C(O)NH2, —C(O)NH(C₁-C₃alkyl), —C(O)N(C₁-C₃alkyl)₂, and hydroxy;
R⁴ is —NHR⁵⁰ or 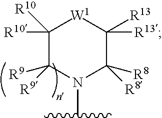
wherein
R⁵⁰ is a five membered ring optionally containing one or two heteroatoms independently selected from the group consisting of nitrogen and oxygen, wherein the ring is optionally substituted with one or two groups independently selected from C₁-C₃alkyl and oxo;
n′ is 0, 1, 2, or 3;
R⁸, R^{8′}, R⁹, R^{9′}, R¹⁰, R^{10′}, R¹³, and R^{13′} are each independently selected from the group consisting of hydrogen, C₁-C₃alkoxy, C₁-C₆alkoxyC₁-C₆alkyl, C₁-C₃alkyl, C₃-C₆cycloalkyl, cyano, cyanoC₁-C₃alkyl, halo, haloC₁-C₃alkyl, hydroxy, and hydroxyC₁-C₃alkyl; or
R⁸ and R⁹, together form a C₁-C₃alkylene; or
R⁸ and R¹⁰ together form C₁-C₃alkylene; or
R⁸ and R¹³ together form C₁-C₃alkylene; or
R⁹ and R¹³ together form C₁-C₃alkylene; or
R¹⁰ and R¹³ together form C₁-C₃alkylene; or
R⁸ and R^{8′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R⁹ and R^{9′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R¹⁰ and R^{10′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R¹³ and R^{13′}, together with the atoms to which they are attached, form a three-, four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy;
W¹ is CR¹¹R¹², NR¹⁷, NR^{15″}C(O), N(C(O)(CH₂)ₙOR¹⁵), O, SO₂, SO₂NR^{15′}, or P(O)CH₃; wherein
n is 0 or 1;
R¹¹ and R¹² are independently selected from the group consisting of hydrogen, C₁-C₃alkoxy, C₁-C₆alkoxyC₁-C₆alkyl, C₁-C₃alkyl, cyano, dimethylphosphino; dimethylsulfonamide, halo, hydroxy, and methylsulfonyl; or
R¹¹ and R¹², together with the atoms to which they are attached, form a four- or five-membered ring optionally containing an oxygen atom or SO₂ group, wherein the ring is optionally substituted with two groups selected from the group consisting of C₁-C₃alkyl, halo, haloC₁-C₃alkyl, and hydroxy; or
R¹¹ and R¹³, together with the atoms to which they are attached, form a three-, four- or five-membered ring optionally containing one or two heteroatoms independently selected from nitrogen and oxygen, wherein the ring optionally contains one or two double bonds, and wherein the ring is optionally substituted with two groups selected from the group consisting of C₁-C₃alkyl, halo, haloC₁-C₃alkyl, and hydroxy; or
R¹³ and R¹⁵, together form CH₂;
R_{15′} is C₁-C₃alkyl or C₁-C₆alkoxyC₁-C₆alkyl;
R^{15″} is hydrogen or C₁-C₃alkyl;
R¹⁷ is selected from the group consisting of C₁-C₃alkylcarbonyl, C₃-C₆cycloalkylcarbonyl, haloC₁-C₃alkylcarbonyl, methylsulfonyl, and tetrahydropyranylcarbonyl, wherein the C₃-C₆cycloalkyl and the tetrahydropyranyl are optionally substituted with one or two substituents independently selected from the group consisting of cyano, halo, and hydroxy;
X is O or NR¹⁶, wherein R¹⁶ is hydrogen or C₁-C₃alkyl;
R⁵ is selected from the group consisting of hydrogen, C₁-C₆alkoxyC₁-C₆alkyl, C₁-C₆alkyl, aryl, arylC₁-C₆alkyl, carboxyC₁-C₆alkyl, C₃-C₆cycloalkyl, C₃-C₆cycloalkylC₁-C₆alkyl, di(C₁-C₃alkyl)aminoC₂-C₆alkyl, haloC₁-C₆alkyl, heteroaryl, heteroarylC₁-C₆alkyl, heterocyclyl, heterocyclylC₁-C₆alkyl, hydroxyC₁-C₆alkyl, NR^{a}R^{b}—C(O)—C₁-C₆alkyl), NR^{a}R^{b}C₁-C₆alkyl, wherein the aryl, the aryl part of the arylC₁-C₆alkyl, the C₃-C₆cycloalkyl, the cycloalkyl part of the C₃-C₆cycloalkylC₁-C₆alkyl, the heteroaryl, the heteroaryl part of the heteroarylC₁-C₆alkyl, the heterocyclyl, the heterocyclyl part of the heterocyclylC₁-C₆alkyl, are optionally substituted with one, two, three, or four groups independently selected from the group consisting of C₁-C₃alkoxy, C₁-C₃alkyl, deuterated C₁-C₃alkyl, C₃-C₆cycloalkyl, (C₁-C₆alkyl)amino, (C₁-C₆alkyl)aminoC₁-C₃alkyl, amino, aminoC₁-C₃alkyl, carboxy, cyano, di(C₁-C₆alkyl)amino, di(C₁-C₆alkyl)aminoC₁-C₃alkyl, halo, haloC₁-C₃alkoxy, haloC₁-C₃alkyl, heterocyclyl, heterocyclylC₁-C₃alkyl, hydroxy, hydroxyC₁-C₃alkyl, nitro, and oxo; wherein the heterocyclyl and the heterocyclyl part of the heterocyclylC₁-C₃alkyl is further optionally substituted with one, two, or three groups independently selected from the group consisting of C₁-C₃alkoxy, C₁-C₃alkyl, halo, and haloC₁-C₃alkyl; or
R⁵ and R¹⁶, together with the nitrogen atom to which they are attached, form a heterocyclic group optionally substituted with one, two, three, four, or five groups independently selected from the group consisting of one, two, three, or four groups independently selected from the group consisting of C₁-C₃alkoxy, C₁-C₃alkoxyalkyl, C₁-C₃alkyl, amino, aminoC₁-C₃alkyl, hydroxy, and hydroxyC₁-C₃alkyl; and
one of R^{a} and R^{b} is selected from hydrogen and C₁-C₃alkyl and the other is selected from the group consisting of hydrogen, C₁-C₃alkyl, C₁-C₃alkoxycarbonyl, C₁-C₃alkylcarbonyl, arylC₁-C₆alkyl, C₃-C₆cycloalkyl, and C₃-C₆cycloalkylC₁-C₆alkyl.

5. The compound of claim 4, or a pharmaceutically acceptable salt thereof, wherein R⁴ is —NHR⁵⁰, and R⁵⁰ is a five membered ring optionally containing one or two heteroatoms independently selected from the group consisting of nitrogen and oxygen, wherein the ring is optionally substituted with one or two groups independently selected from C₁-C₃alkyl and oxo.

6.-13. (canceled)

14. The compound of claim 4, or a pharmaceutically acceptable salt thereof, wherein R⁴ is 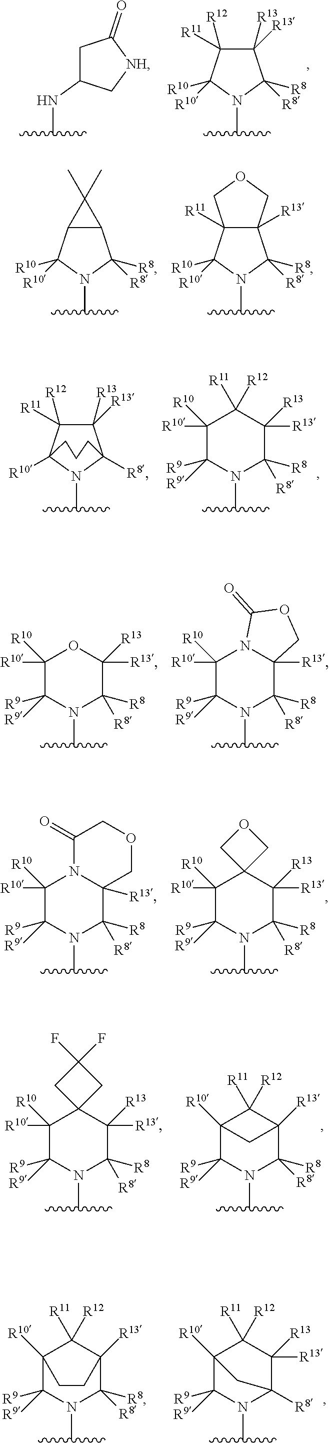 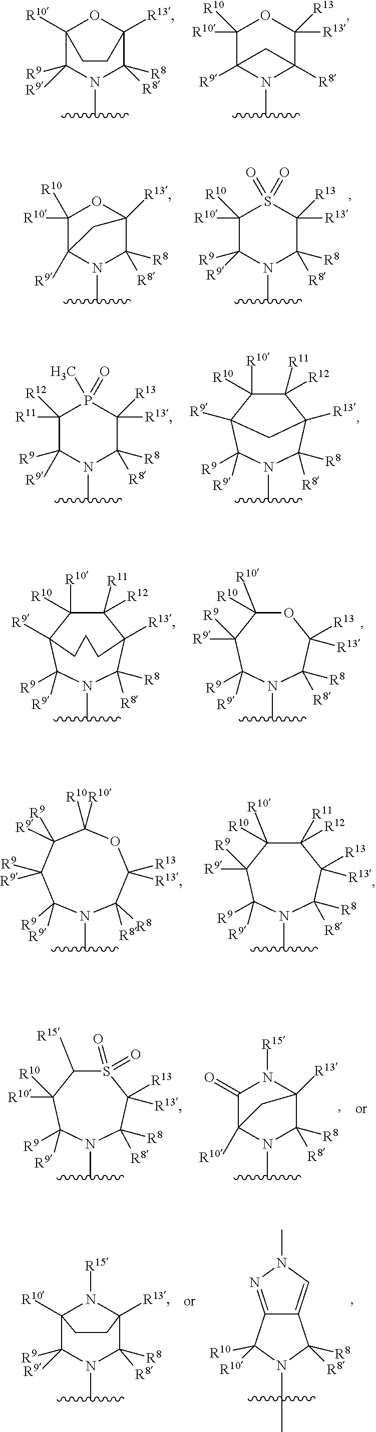
wherein [symbol] represents the point of attachment to the core of formula (I) or (II).

15. The compound of claim 4, or a pharmaceutically acceptable salt thereof, wherein R⁴ is 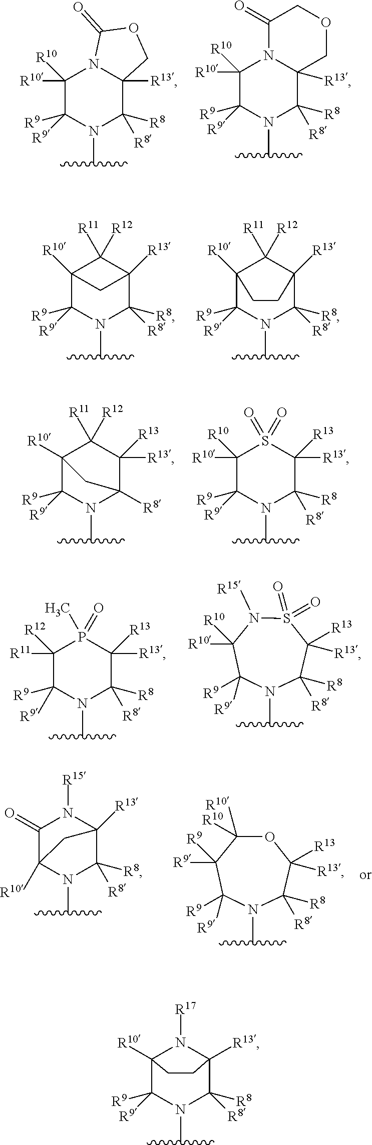
wherein [symbol] represents the point of attachment to the core of formula (II).

16.-18. (canceled)

19. The compound of claim 4, or a pharmaceutically acceptable salt thereof, wherein R⁵ is selected from the group consisting of 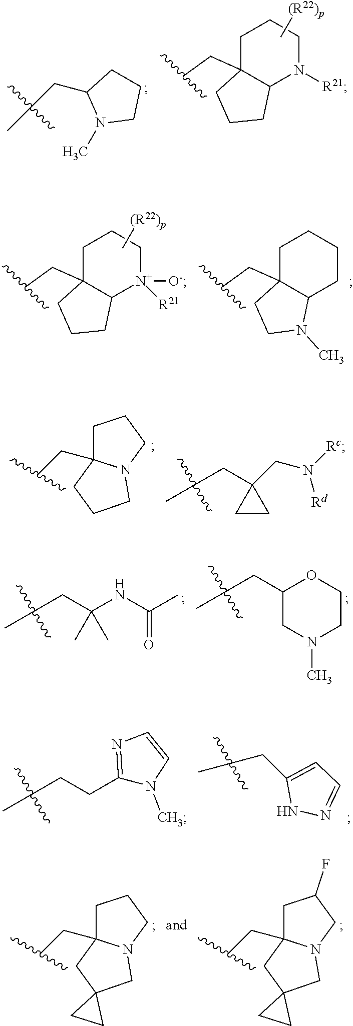
wherein each ring is optionally substituted with 1, 2, or 3 groups independently selected from the group consisting of C₁-C₃alkoxy, C₁-C₃alkoxyC₁-C₃alkyl, C₁-C₃alkyl, deuterated C₁-C₃alkyl, C₃-C₆cycloalkyl, benzyl, halo, haloC₁-C₃alkyl, hydroxy, hydroxyC₁-C₃alkyl, and oxo; and wherein
R^{c} and R^{d}, together with the nitrogen atom to which they are attached, form a five- to ten-membered ring monocyclic or bicyclic ring optionally containing one additional heteroatom selected from nitrogen, oxygen, and sulfur, wherein the ring is optionally substituted with one, two, or three groups independently selected from C₁-C₃alkoxy, C₁-C₃alkoxyC₁-C₃alkyl, C₁-C₃alkyl, becnzyl, halo, haloC1-C₃alkyl, hydroxy, hydroxyC₁-C₃alkyl, and oxo; or
one of R^{c} and R^{d} is selected from hydrogen and C₁-C₃alkyl and the other is selected from hydrogen, C₁-C₃alkyl, C₁-C₃alkoxycarbonyl, and C₁-C₃alkylcarbonyl.

20. The compound of claim 4, or a pharmaceutically acceptable salt thereof, wherein R⁵ is —(C₁-C₃alkyl)-R⁶, wherein R⁶ is a three- to six-membered monocyclic ring system, an eight- or nine-membered bicyclic fused saturated ring system, a ten-membered tricyclic saturated ring system, or a twelve-membered tetracyclic saturated ring system, wherein each ring system optionally contains one or more nitrogen, oxygen and/or sulfur atoms, and wherein each ring system is optionally substituted with one to four groups independently selected from the group consisting of C₁-C₃alkyl, halo, oxo, and (4- to 6-membered heterocyclyl)C₁-C₃alkyl; wherein the heterocyclyl part of the (4- to 6-membered heterocyclyl)C₁-C₃alkyl is further optionally substituted with a halo group.

21.-27. (canceled)

28. The compound of claim 4, or a pharmaceutically acceptable salt thereof, wherein Z is a bond.

29. The compound of claim 4, wherein R¹ is a monocyclic heteroaryl ring containing one, two, or three nitrogen atoms, wherein the ring is optionally substituted with one, two, three, four, or five substituents independently selected from the group consisting of C₁-C₃alkoxy, C₁-C₃alkyl, C₂-C₄alkenyl, C₂-C₄alkynyl, amino, aminoC₁-C₃alkyl, cyano, C₃-C₄cycloalkyl, halo, haloC₁-C₃alkyl, haloC₁-C₃alkoxy, hydroxy, and hydroxyC₁-C₃alkyl.

30. (canceled)

31. The compound of claim 4, or a pharmaceutically acceptable salt thereof, wherein R¹ ics C₆-C₁₀aryl optionally substituted with one, two, three, four, or five substituents independently selected from the group consisting of C₁-C₃alkoxy, C₁-C₃alkyl, C₂-C₄alkenyl, C₂-C₄alkynyl, amino, aminoC₁-C₃alkyl, cyano, C₃-C₅cycloalkyl, halo, haloC₁-C₃alkyl, hydroxy, and hydroxyC₁-C₃alkyl.

32. The compound of claim 4, or a pharmaceutically acceptable salt thereof, wherein R¹ is aryl or heteroaryl, wherein the aryl and heteroaryl are optionally substituted with one, two, three, four, or five substituents independently selected from the group consisting of C₂-C₄alkenyl, C₁-C₃alkoxy, C₁-C₃alkyl, C₂-C₄alkynyl, C₂-C₄alkynyloxy, amino, cyano, cyanoC₁-C₃alkoxy, C₃-C₅cycloalkyl optionally substituted with one or two halo groups, halo, haloC₁-C₃alkyl, haloC₁-C₃alkoxy, 4- to 6-membered heterocyclyl, and hydroxy.

33.-39. (canceled)

40. The compound of claim 1 that is selected from the group consisting of: 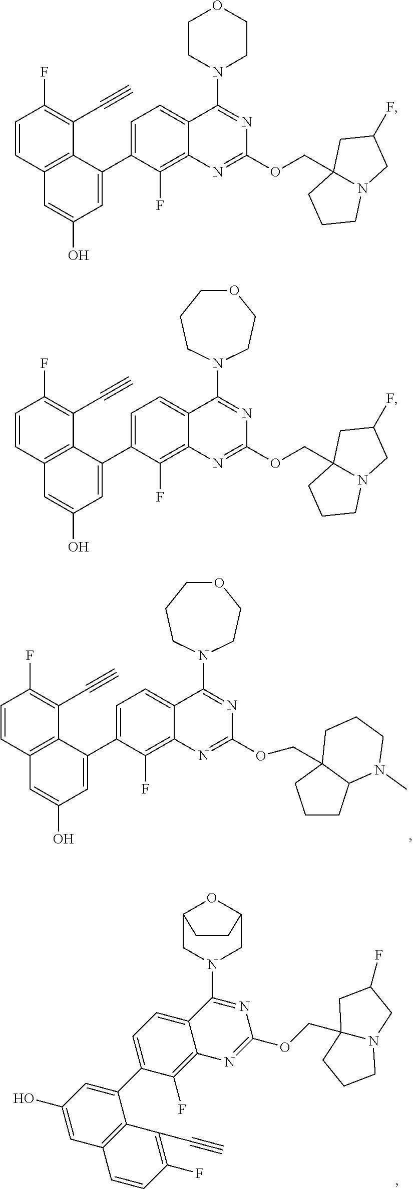 [SMILES: C#Cc1c(F)ccc2cc(O)cc(-c3ccc4c(N5CCOCC5)nc(OCC56CCCN5CC(F)C6)nc4c3F)c12 ; C#Cc1c(F)ccc2cc(O)cc(-c3ccc4c(N5CCCOCC5)nc(OCC56CCCN5CC(F)C6)nc4c3F)c12 ; C#Cc1c(F)ccc2cc(O)cc(-c3ccc4c(N5CCCOCC5)nc(OCC56CCCC5N(C)CCC6)nc4c3F)c12 ; C#Cc1c(F)ccc2cc(O)cc(-c3ccc4c(N5CC6CCC(C5)O6)nc(OCC56CCCN5CC(F)C6)nc4c3F)c12] 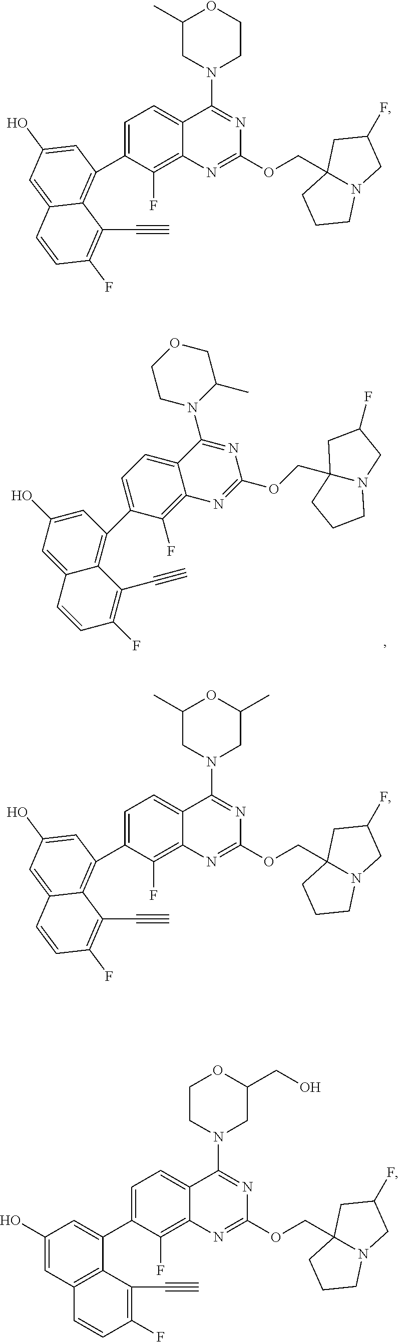 [SMILES: C#Cc1c(F)ccc2cc(O)cc(-c3ccc4c(N5CCOC(C)C5)nc(OCC56CCCN5CC(F)C6)nc4c3F)c12 ; C#Cc1c(F)ccc2cc(O)cc(-c3ccc4c(N5CCOCC5C)nc(OCC56CCCN5CC(F)C6)nc4c3F)c12 ; C#Cc1c(F)ccc2cc(O)cc(-c3ccc4c(N5CC(C)OC(C)C5)nc(OCC56CCCN5CC(F)C6)nc4c3F)c12 ; C#Cc1c(F)ccc2cc(O)cc(-c3ccc4c(N5CCOC(CO)C5)nc(OCC56CCCN5CC(F)C6)nc4c3F)c12] 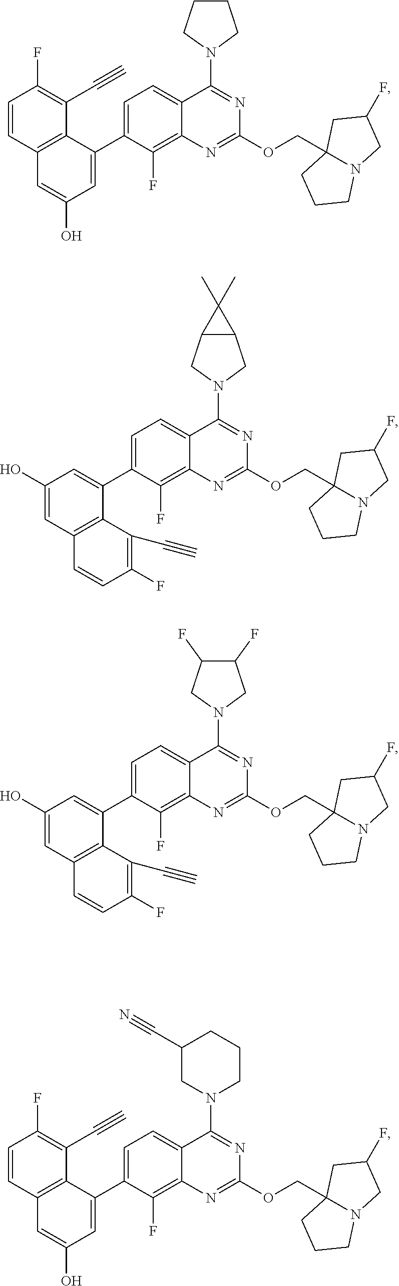 [SMILES: C#Cc1c(F)ccc2cc(O)cc(-c3ccc4c(N5CCCC5)nc(OCC56CCCN5CC(F)C6)nc4c3F)c12 ; C#Cc1c(F)ccc2cc(O)cc(-c3ccc4c(N5CC6C(C5)C6(C)C)nc(OCC56CCCN5CC(F)C6)nc4c3F)c12 ; C#Cc1c(F)ccc2cc(O)cc(-c3ccc4c(N5CC(F)C(F)C5)nc(OCC56CCCN5CC(F)C6)nc4c3F)c12 ; C#Cc1c(F)ccc2cc(O)cc(-c3ccc4c(N5CCCC(C#N)C5)nc(OCC56CCCN5CC(F)C6)nc4c3F)c12] 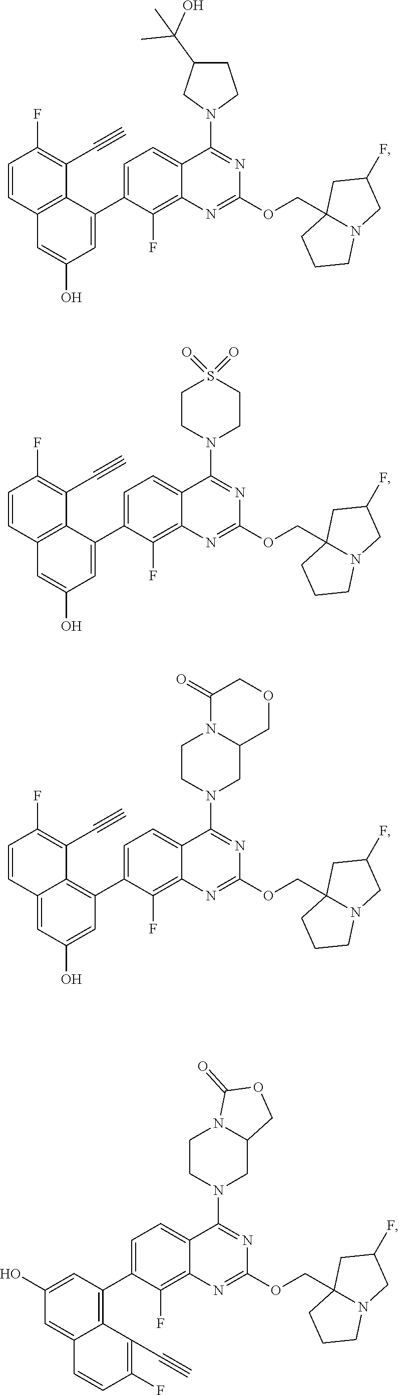 [SMILES: C#Cc1c(F)ccc2cc(O)cc(-c3ccc4c(N5CCC(C(C)(C)O)C5)nc(OCC56CCCN5CC(F)C6)nc4c3F)c12 ; C#Cc1c(F)ccc2cc(O)cc(-c3ccc4c(N5CCS(=O)(=O)CC5)nc(OCC56CCCN5CC(F)C6)nc4c3F)c12 ; C#Cc1c(F)ccc2cc(O)cc(-c3ccc4c(N5CCN6C(=O)COCC6C5)nc(OCC56CCCN5CC(F)C6)nc4c3F)c12 ; C#Cc1c(F)ccc2cc(O)cc(-c3ccc4c(N5CCN6C(=O)OCC6C5)nc(OCC56CCCN5CC(F)C6)nc4c3F)c12] 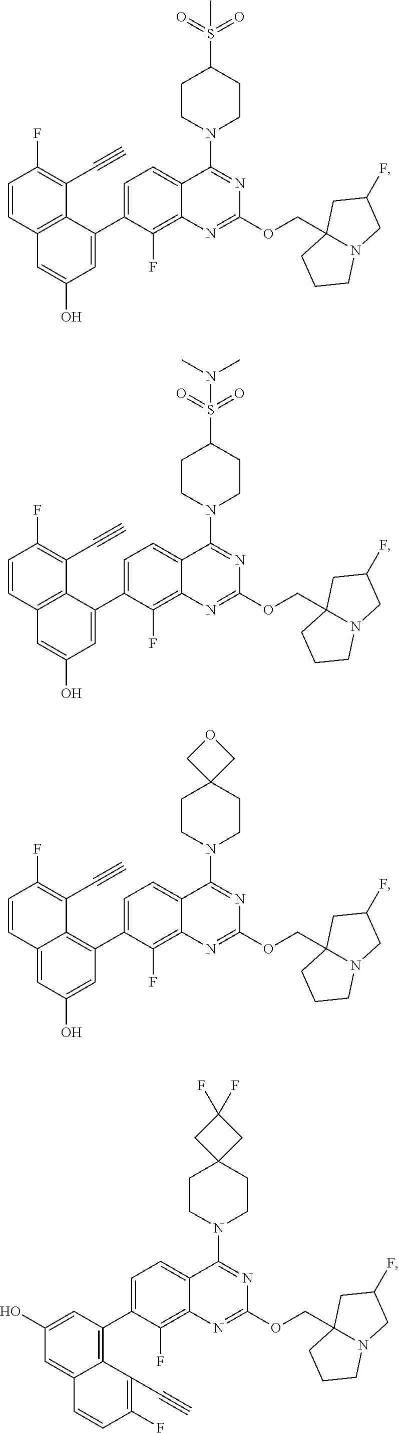 [SMILES: C#Cc1c(F)ccc2cc(O)cc(-c3ccc4c(N5CCC(S(C)(=O)=O)CC5)nc(OCC56CCCN5CC(F)C6)nc4c3F)c12 ; C#Cc1c(F)ccc2cc(O)cc(-c3ccc4c(N5CCC(S(=O)(=O)N(C)C)CC5)nc(OCC56CCCN5CC(F)C6)nc4c3F)c12 ; C#Cc1c(F)ccc2cc(O)cc(-c3ccc4c(N5CCC6(CC5)COC6)nc(OCC56CCCN5CC(F)C6)nc4c3F)c12 ; C#Cc1c(F)ccc2cc(O)cc(-c3ccc4c(N5CCC6(CC5)CC(F)(F)C6)nc(OCC56CCCN5CC(F)C6)nc4c3F)c12] 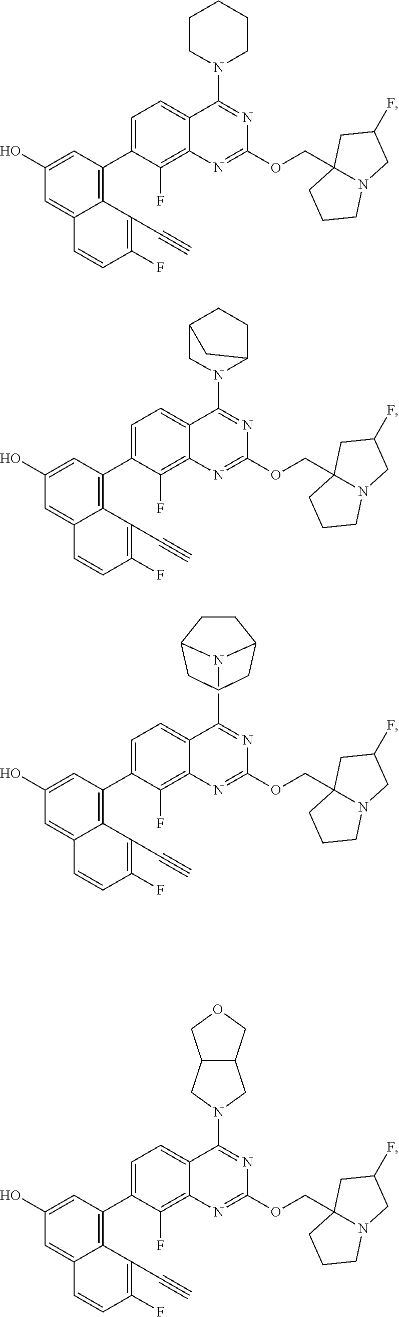 [SMILES: C#Cc1c(F)ccc2cc(O)cc(-c3ccc4c(N5CCCCC5)nc(OCC56CCCN5CC(F)C6)nc4c3F)c12 ; C#Cc1c(F)ccc2cc(O)cc(-c3ccc4c(N5CC6CCC5C6)nc(OCC56CCCN5CC(F)C6)nc4c3F)c12 ; C#Cc1c(F)ccc2cc(O)cc(-c3ccc4c(N5C6CCCC5CC6)nc(OCC56CCCN5CC(F)C6)nc4c3F)c12 ; C#Cc1c(F)ccc2cc(O)cc(-c3ccc4c(N5CC6COCC6C5)nc(OCC56CCCN5CC(F)C6)nc4c3F)c12] 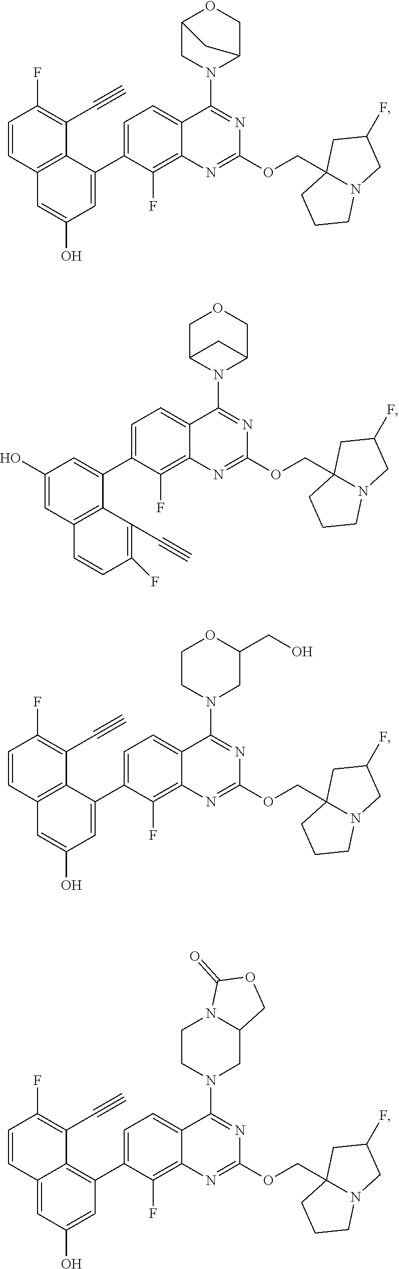 [SMILES: C#Cc1c(F)ccc2cc(O)cc(-c3ccc4c(N5CC6CC5CO6)nc(OCC56CCCN5CC(F)C6)nc4c3F)c12 ; C#Cc1c(F)ccc2cc(O)cc(-c3ccc4c(N5C6COCC5C6)nc(OCC56CCCN5CC(F)C6)nc4c3F)c12 ; C#Cc1c(F)ccc2cc(O)cc(-c3ccc4c(N5CCOC(CO)C5)nc(OCC56CCCN5CC(F)C6)nc4c3F)c12 ; C#Cc1c(F)ccc2cc(O)cc(-c3ccc4c(N5CCN6C(=O)OCC6C5)nc(OCC56CCCN5CC(F)C6)nc4c3F)c12] 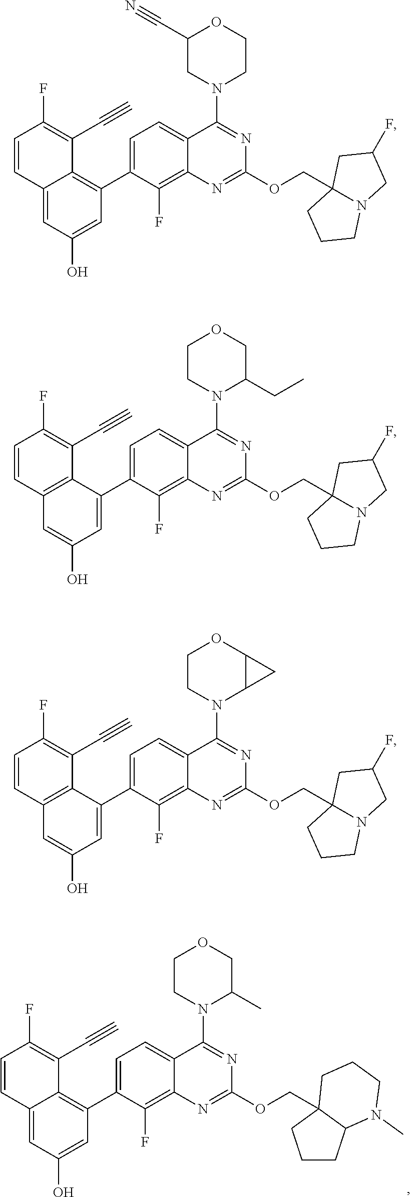 [SMILES: C#Cc1c(F)ccc2cc(O)cc(-c3ccc4c(N5CCOC(C#N)C5)nc(OCC56CCCN5CC(F)C6)nc4c3F)c12 ; C#Cc1c(F)ccc2cc(O)cc(-c3ccc4c(N5CCOCC5CC)nc(OCC56CCCN5CC(F)C6)nc4c3F)c12 ; C#Cc1c(F)ccc2cc(O)cc(-c3ccc4c(N5CCOC6CC65)nc(OCC56CCCN5CC(F)C6)nc4c3F)c12 ; C#Cc1c(F)ccc2cc(O)cc(-c3ccc4c(N5CCOCC5C)nc(OCC56CCCC5N(C)CCC6)nc4c3F)c12] 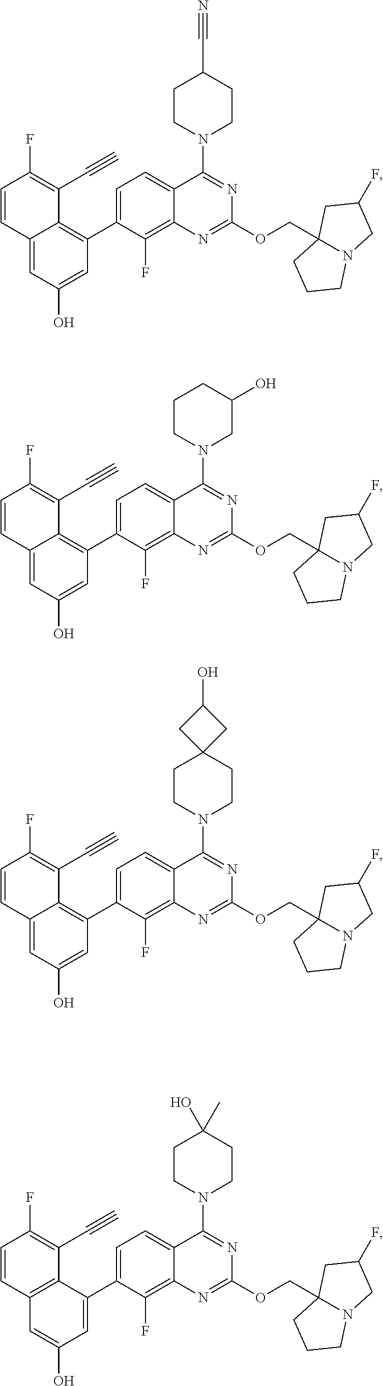 [SMILES: C#Cc1c(F)ccc2cc(O)cc(-c3ccc4c(N5CCC(C#N)CC5)nc(OCC56CCCN5CC(F)C6)nc4c3F)c12 ; C#Cc1c(F)ccc2cc(O)cc(-c3ccc4c(N5CCCC(O)C5)nc(OCC56CCCN5CC(F)C6)nc4c3F)c12 ; C#Cc1c(F)ccc2cc(O)cc(-c3ccc4c(N5CCC6(CC5)CC(O)C6)nc(OCC56CCCN5CC(F)C6)nc4c3F)c12 ; C#Cc1c(F)ccc2cc(O)cc(-c3ccc4c(N5CCC(C)(O)CC5)nc(OCC56CCCN5CC(F)C6)nc4c3F)c12] 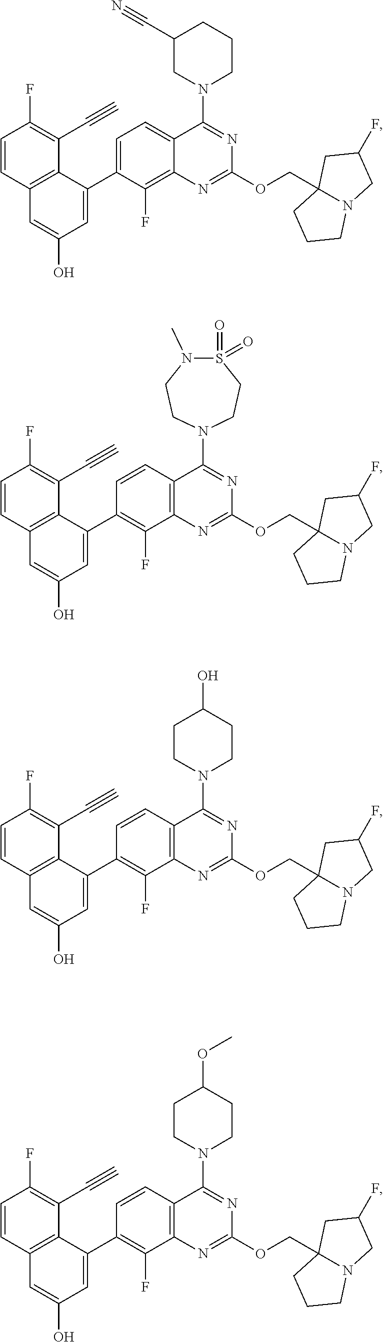 [SMILES: C#Cc1c(F)ccc2cc(O)cc(-c3ccc4c(N5CCCC(C#N)C5)nc(OCC56CCCN5CC(F)C6)nc4c3F)c12 ; C#Cc1c(F)ccc2cc(O)cc(-c3ccc4c(N5CCN(C)S(=O)(=O)CC5)nc(OCC56CCCN5CC(F)C6)nc4c3F)c12 ; C#Cc1c(F)ccc2cc(O)cc(-c3ccc4c(N5CCC(O)CC5)nc(OCC56CCCN5CC(F)C6)nc4c3F)c12 ; C#Cc1c(F)ccc2cc(O)cc(-c3ccc4c(N5CCC(OC)CC5)nc(OCC56CCCN5CC(F)C6)nc4c3F)c12] 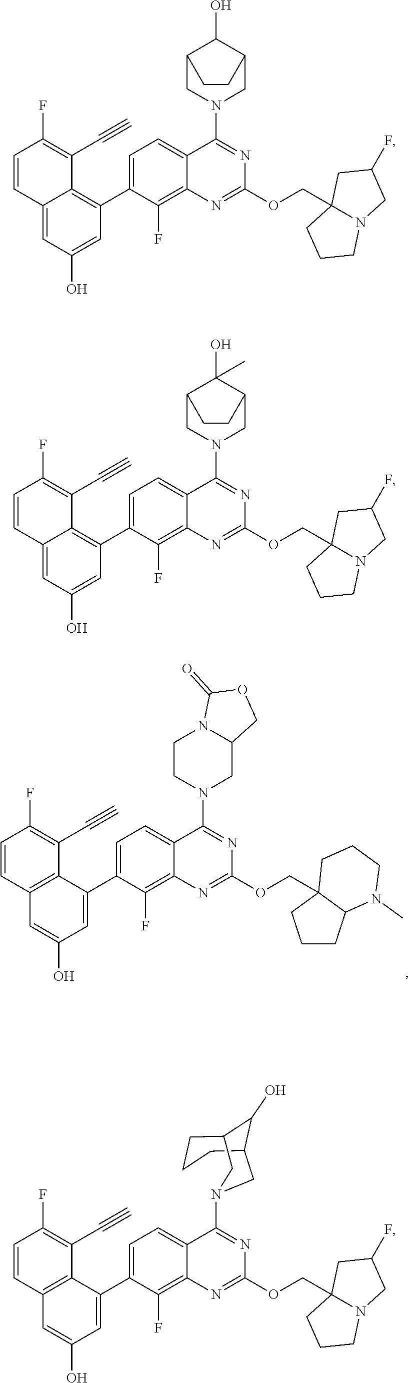 [SMILES: C#Cc1c(F)ccc2cc(O)cc(-c3ccc4c(N5CC6CCC(C5)C6O)nc(OCC56CCCN5CC(F)C6)nc4c3F)c12 ; C#Cc1c(F)ccc2cc(O)cc(-c3ccc4c(N5CC6CCC(C5)C6(C)O)nc(OCC56CCCN5CC(F)C6)nc4c3F)c12 ; C#Cc1c(F)ccc2cc(O)cc(-c3ccc4c(N5CCN6C(=O)OCC6C5)nc(OCC56CCCC5N(C)CCC6)nc4c3F)c12 ; C#Cc1c(F)ccc2cc(O)cc(-c3ccc4c(N5CC6CCCC(C5)C6O)nc(OCC56CCCN5CC(F)C6)nc4c3F)c12] 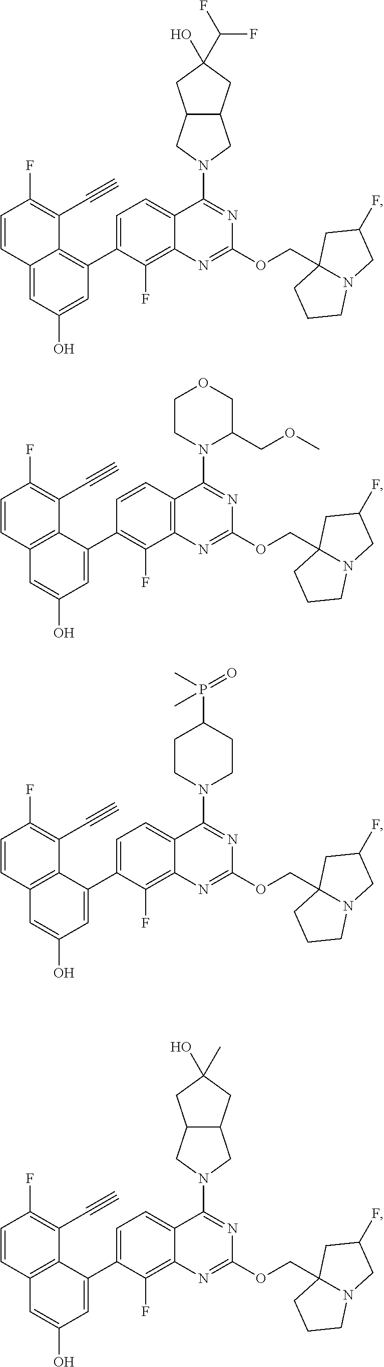 [SMILES: C#Cc1c(F)ccc2cc(O)cc(-c3ccc4c(N5CC6CC(O)(C(F)F)CC6C5)nc(OCC56CCCN5CC(F)C6)nc4c3F)c12 ; C#Cc1c(F)ccc2cc(O)cc(-c3ccc4c(N5CCOCC5COC)nc(OCC56CCCN5CC(F)C6)nc4c3F)c12 ; C#Cc1c(F)ccc2cc(O)cc(-c3ccc4c(N5CCC(P(C)(C)=O)CC5)nc(OCC56CCCN5CC(F)C6)nc4c3F)c12 ; C#Cc1c(F)ccc2cc(O)cc(-c3ccc4c(N5CC6CC(C)(O)CC6C5)nc(OCC56CCCN5CC(F)C6)nc4c3F)c12] 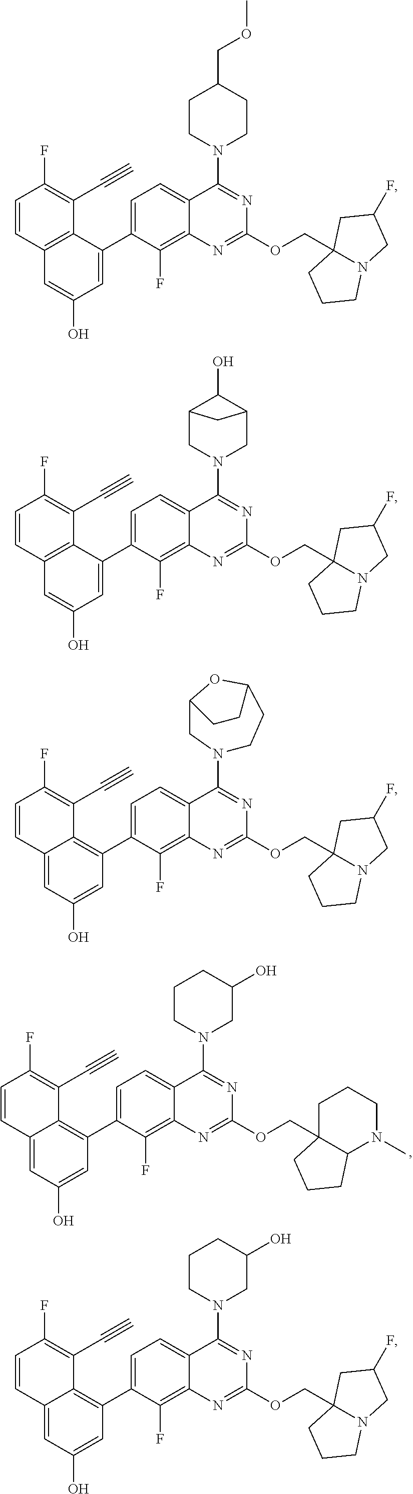 [SMILES: C#Cc1c(F)ccc2cc(O)cc(-c3ccc4c(N5CCC(COC)CC5)nc(OCC56CCCN5CC(F)C6)nc4c3F)c12 ; C#Cc1c(F)ccc2cc(O)cc(-c3ccc4c(N5CC6CC(C5)C6O)nc(OCC56CCCN5CC(F)C6)nc4c3F)c12 ; C#Cc1c(F)ccc2cc(O)cc(-c3ccc4c(N5CCC6CCC(C5)O6)nc(OCC56CCCN5CC(F)C6)nc4c3F)c12 ; C#Cc1c(F)ccc2cc(O)cc(-c3ccc4c(N5CCCC(O)C5)nc(OCC56CCCC5N(C)CCC6)nc4c3F)c12 ; C#Cc1c(F)ccc2cc(O)cc(-c3ccc4c(N5CCCC(O)C5)nc(OCC56CCCN5CC(F)C6)nc4c3F)c12] 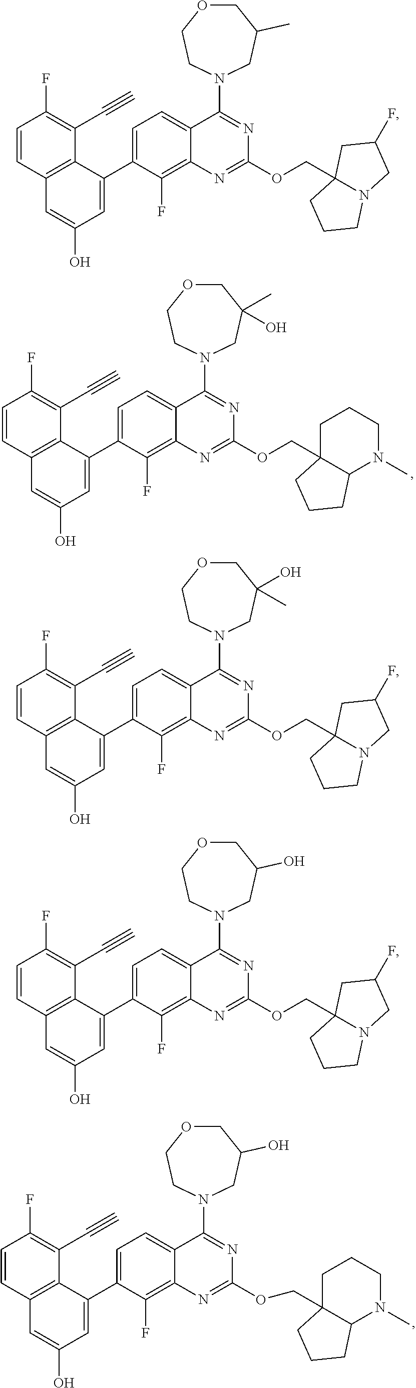 [SMILES: C#Cc1c(F)ccc2cc(O)cc(-c3ccc4c(N5CCOCC(C)C5)nc(OCC56CCCN5CC(F)C6)nc4c3F)c12 ; C#Cc1c(F)ccc2cc(O)cc(-c3ccc4c(N5CCOCC(C)(O)C5)nc(OCC56CCCC5N(C)CCC6)nc4c3F)c12 ; C#Cc1c(F)ccc2cc(O)cc(-c3ccc4c(N5CCOCC(C)(O)C5)nc(OCC56CCCN5CC(F)C6)nc4c3F)c12 ; C#Cc1c(F)ccc2cc(O)cc(-c3ccc4c(N5CCOCC(O)C5)nc(OCC56CCCN5CC(F)C6)nc4c3F)c12 ; C#Cc1c(F)ccc2cc(O)cc(-c3ccc4c(N5CCOCC(O)C5)nc(OCC56CCCC5N(C)CCC6)nc4c3F)c12] 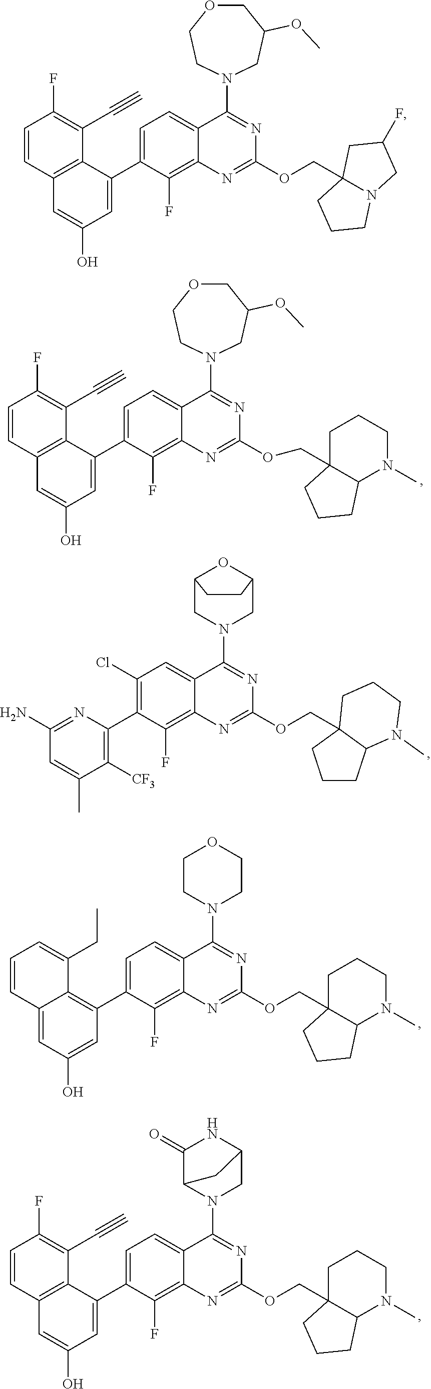 [SMILES: C#Cc1c(F)ccc2cc(O)cc(-c3ccc4c(N5CCOCC(OC)C5)nc(OCC56CCCN5CC(F)C6)nc4c3F)c12 ; C#Cc1c(F)ccc2cc(O)cc(-c3ccc4c(N5CCOCC(OC)C5)nc(OCC56CCCC5N(C)CCC6)nc4c3F)c12 ; Cc1cc(N)nc(-c2c(Cl)cc3c(N4CC5CCC(C4)O5)nc(OCC45CCCC4N(C)CCC5)nc3c2F)c1C(F)(F)F ; CCc1cccc2cc(O)cc(-c3ccc4c(N5CCOCC5)nc(OCC56CCCC5N(C)CCC6)nc4c3F)c12 ; C#Cc1c(F)ccc2cc(O)cc(-c3ccc4c(N5CC6CC5C(=O)N6)nc(OCC56CCCC5N(C)CCC6)nc4c3F)c12] 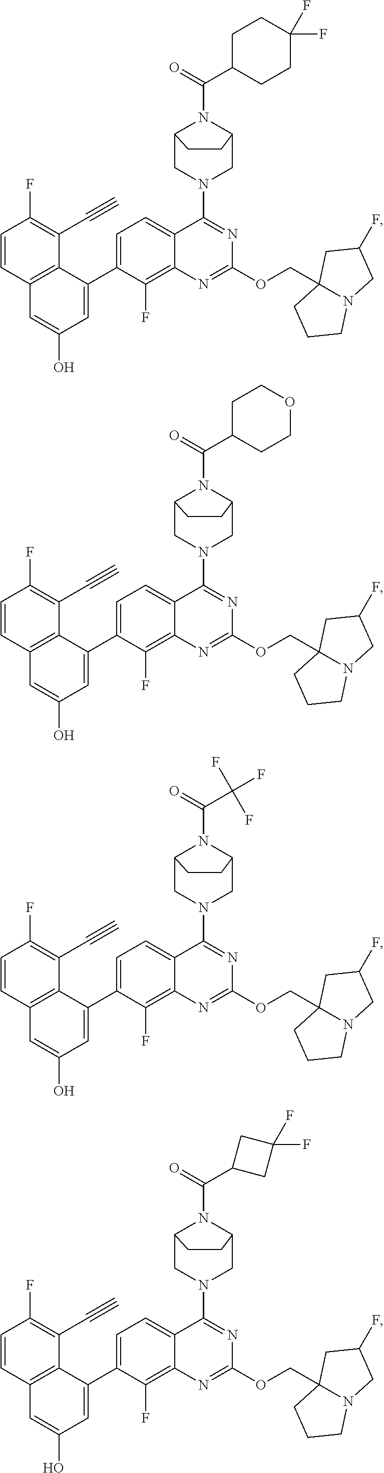 [SMILES: C#Cc1c(F)ccc2cc(O)cc(-c3ccc4c(N5CC6CCC(C5)N6C(=O)C5CCC(F)(F)CC5)nc(OCC56CCCN5CC(F)C6)nc4c3F)c12 ; C#Cc1c(F)ccc2cc(O)cc(-c3ccc4c(N5CC6CCC(C5)N6C(=O)C5CCOCC5)nc(OCC56CCCN5CC(F)C6)nc4c3F)c12 ; C#Cc1c(F)ccc2cc(O)cc(-c3ccc4c(N5CC6CCC(C5)N6C(=O)C(F)(F)F)nc(OCC56CCCN5CC(F)C6)nc4c3F)c12 ; C#Cc1c(F)ccc2cc(O)cc(-c3ccc4c(N5CC6CCC(C5)N6C(=O)C5CC(F)(F)C5)nc(OCC56CCCN5CC(F)C6)nc4c3F)c12] 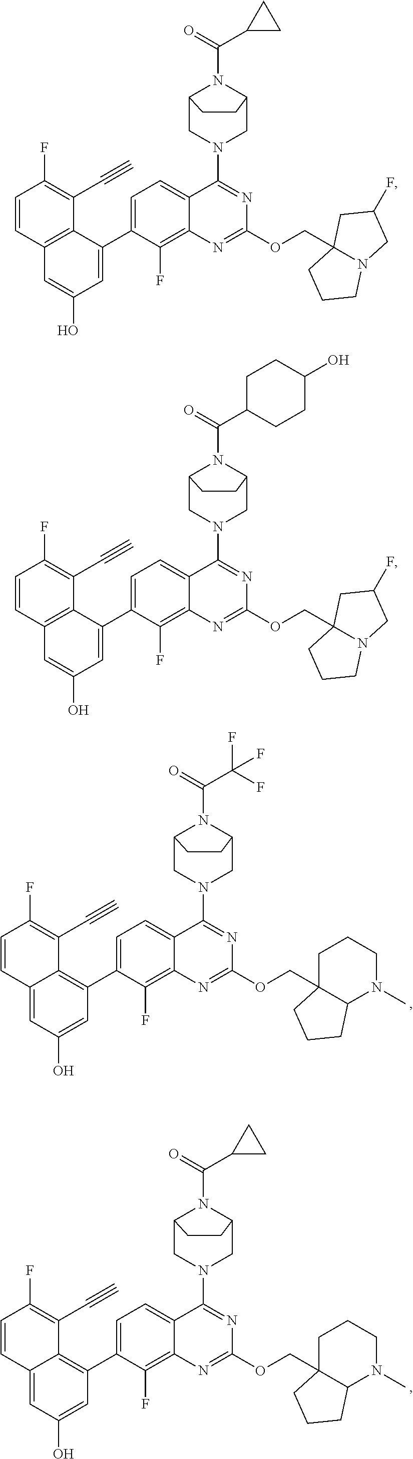 [SMILES: C#Cc1c(F)ccc2cc(O)cc(-c3ccc4c(N5CC6CCC(C5)N6C(=O)C5CC5)nc(OCC56CCCN5CC(F)C6)nc4c3F)c12 ; C#Cc1c(F)ccc2cc(O)cc(-c3ccc4c(N5CC6CCC(C5)N6C(=O)C5CCC(O)CC5)nc(OCC56CCCN5CC(F)C6)nc4c3F)c12 ; C#Cc1c(F)ccc2cc(O)cc(-c3ccc4c(N5CC6CCC(C5)N6C(=O)C(F)(F)F)nc(OCC56CCCC5N(C)CCC6)nc4c3F)c12 ; C#Cc1c(F)ccc2cc(O)cc(-c3ccc4c(N5CC6CCC(C5)N6C(=O)C5CC5)nc(OCC56CCCC5N(C)CCC6)nc4c3F)c12] 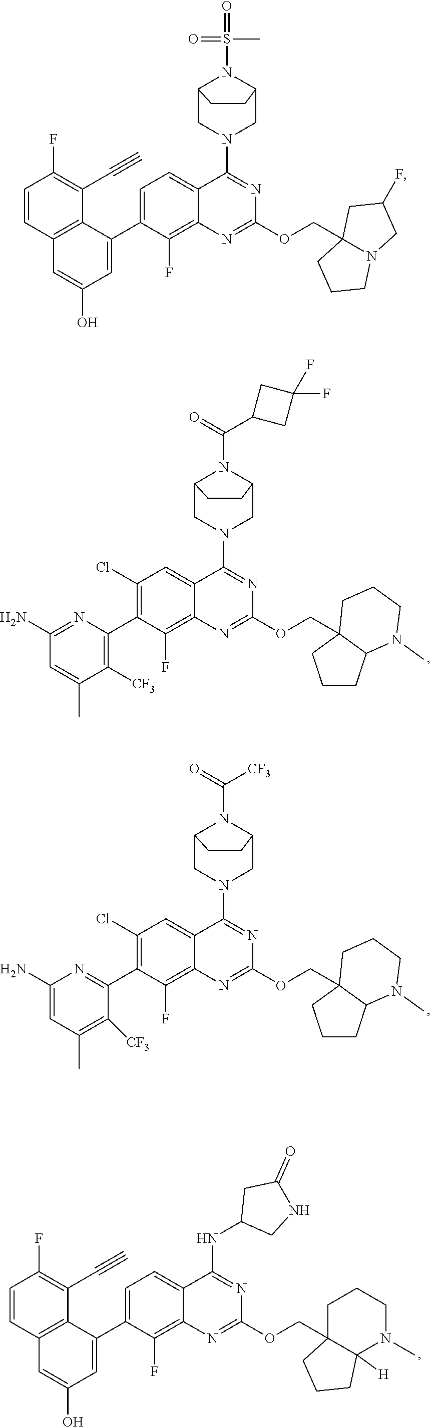 [SMILES: C#Cc1c(F)ccc2cc(O)cc(-c3ccc4c(N5CC6CCC(C5)N6S(C)(=O)=O)nc(OCC56CCCN5CC(F)C6)nc4c3F)c12 ; Cc1cc(N)nc(-c2c(Cl)cc3c(N4CC5CCC(C4)N5C(=O)C4CC(F)(F)C4)nc(OCC45CCCC4N(C)CCC5)nc3c2F)c1C(F)(F)F ; Cc1cc(N)nc(-c2c(Cl)cc3c(N4CC5CCC(C4)N5C(=O)C(F)(F)F)nc(OCC45CCCC4N(C)CCC5)nc3c2F)c1C(F)(F)F ; C#Cc1c(F)ccc2cc(O)cc(-c3ccc4c(NC5CNC(=O)C5)nc(OCC56CCCC5N(C)CCC6)nc4c3F)c12] 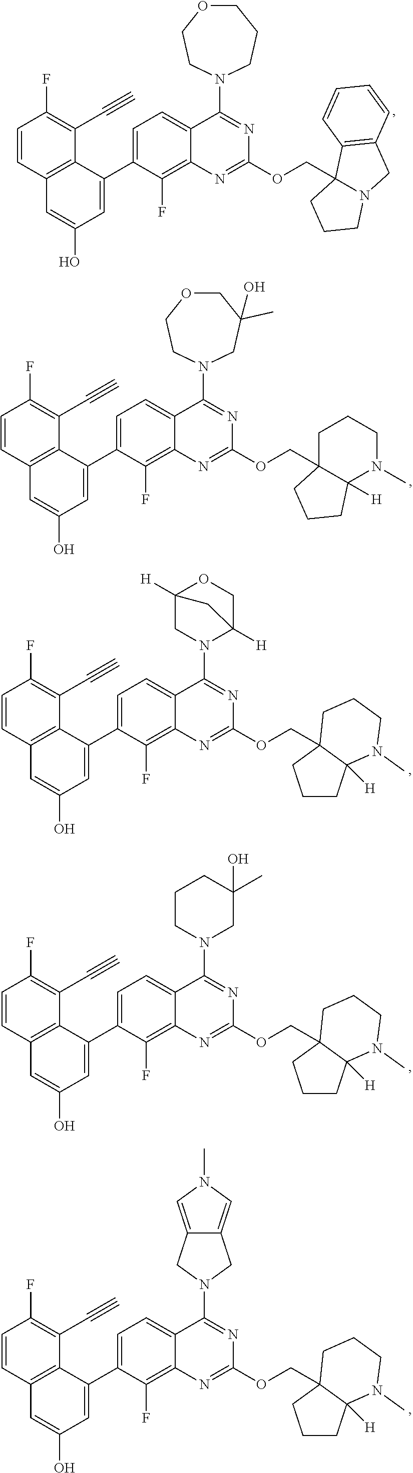 [SMILES: C#Cc1c(F)ccc2cc(O)cc(-c3ccc4c(N5CCCOCC5)nc(OCC56CCCN5Cc5ccccc56)nc4c3F)c12 ; C#Cc1c(F)ccc2cc(O)cc(-c3ccc4c(N5CCOCC(C)(O)C5)nc(OCC56CCCC5N(C)CCC6)nc4c3F)c12 ; C#Cc1c(F)ccc2cc(O)cc(-c3ccc4c(N5CC6CC5CO6)nc(OCC56CCCC5N(C)CCC6)nc4c3F)c12 ; C#Cc1c(F)ccc2cc(O)cc(-c3ccc4c(N5CCCC(C)(O)C5)nc(OCC56CCCC5N(C)CCC6)nc4c3F)c12 ; C#Cc1c(F)ccc2cc(O)cc(-c3ccc4c(N5Cc6cn(C)cc6C5)nc(OCC56CCCC5N(C)CCC6)nc4c3F)c12] 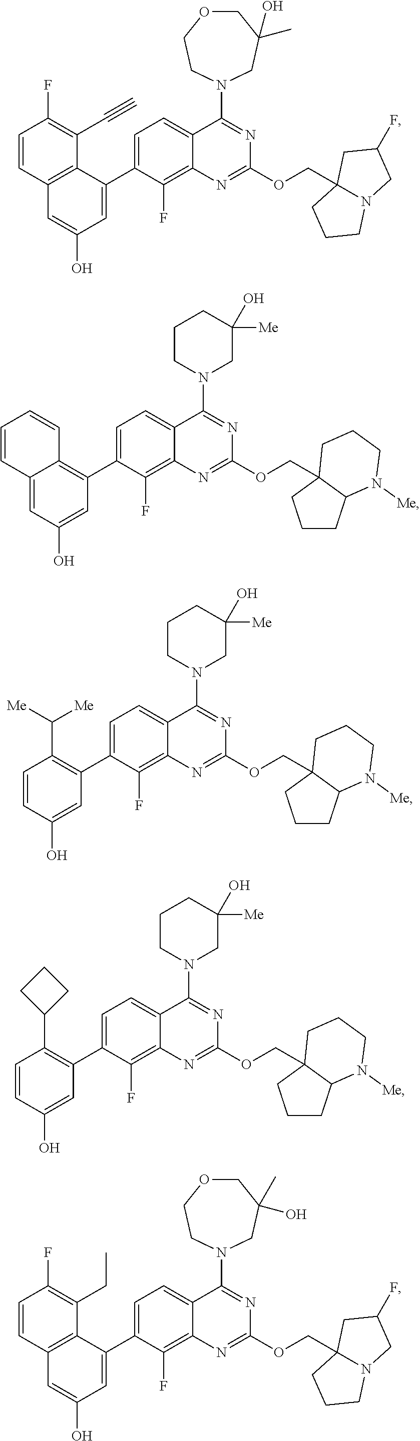 [SMILES: C#Cc1c(F)ccc2cc(O)cc(-c3ccc4c(N5CCOCC(C)(O)C5)nc(OCC56CCCN5CC(F)C6)nc4c3F)c12 ; CN1CCCC2(COc3nc(N4CCCC(C)(O)C4)c4ccc(-c5cc(O)cc6ccccc56)c(F)c4n3)CCCC12 ; CC(C)c1ccc(O)cc1-c1ccc2c(N3CCCC(C)(O)C3)nc(OCC34CCCC3N(C)CCC4)nc2c1F ; CN1CCCC2(COc3nc(N4CCCC(C)(O)C4)c4ccc(-c5cc(O)ccc5C5CCC5)c(F)c4n3)CCCC12 ; CCc1c(F)ccc2cc(O)cc(-c3ccc4c(N5CCOCC(C)(O)C5)nc(OCC56CCCN5CC(F)C6)nc4c3F)c12] 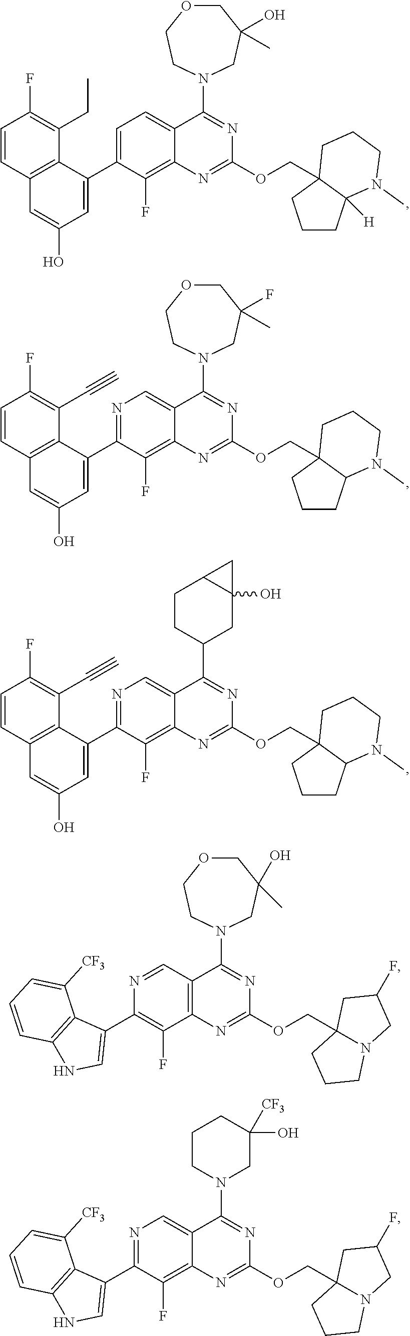 [SMILES: CCc1c(F)ccc2cc(O)cc(-c3ccc4c(N5CCOCC(C)(O)C5)nc(OCC56CCCC5N(C)CCC6)nc4c3F)c12 ; C#Cc1c(F)ccc2cc(O)cc(-c3ncc4c(N5CCOCC(C)(F)C5)nc(OCC56CCCC5N(C)CCC6)nc4c3F)c12 ; C#Cc1c(F)ccc2cc(O)cc(-c3ncc4c(C5CCC6CC6(O)C5)nc(OCC56CCCC5N(C)CCC6)nc4c3F)c12 ; CC1(O)COCCN(c2nc(OCC34CCCN3CC(F)C4)nc3c(F)c(-c4c[nH]c5cccc(C(F)(F)F)c45)ncc23)C1 ; OC1(C(F)(F)F)CCCN(c2nc(OCC34CCCN3CC(F)C4)nc3c(F)c(-c4c[nH]c5cccc(C(F)(F)F)c45)ncc23)C1] 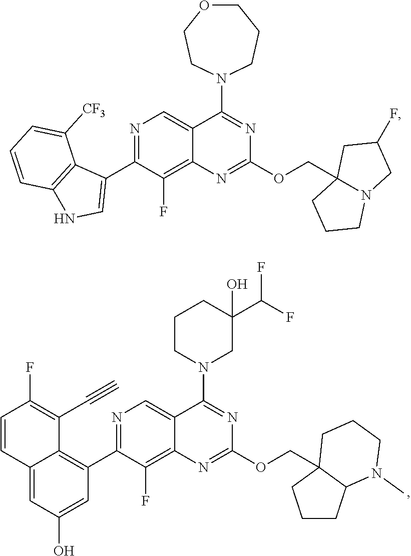 [SMILES: Fc1c(-c2c[nH]c3cccc(C(F)(F)F)c23)ncc2c(N3CCCOCC3)nc(OCC34CCCN3CC(F)C4)nc12 ; C#Cc1c(F)ccc2cc(O)cc(-c3ncc4c(N5CCCC(O)(C(F)F)C5)nc(OCC56CCCC5N(C)CCC6)nc4c3F)c12] 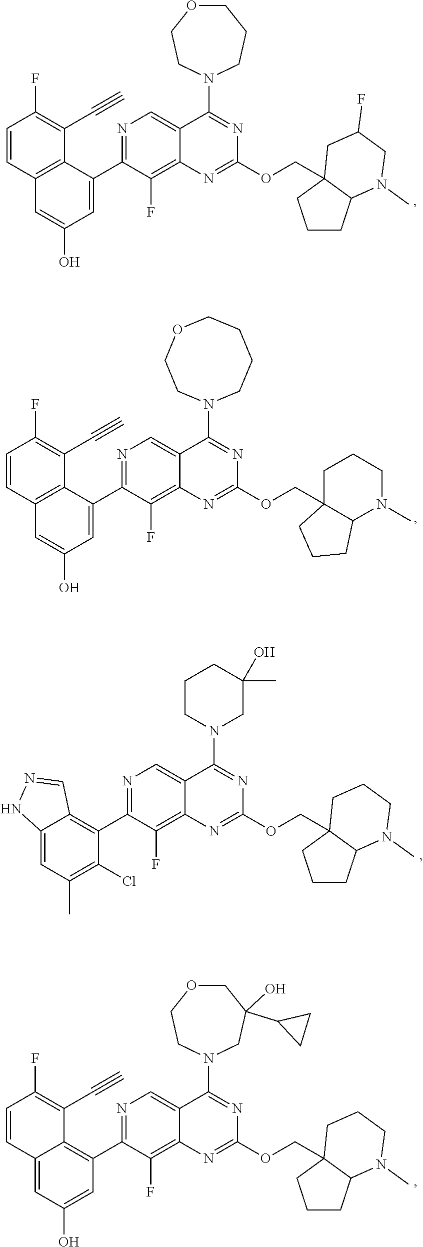 [SMILES: C#Cc1c(F)ccc2cc(O)cc(-c3ncc4c(N5CCCOCC5)nc(OCC56CCCC5N(C)CC(F)C6)nc4c3F)c12 ; C#Cc1c(F)ccc2cc(O)cc(-c3ncc4c(N5CCCCOCC5)nc(OCC56CCCC5N(C)CCC6)nc4c3F)c12 ; Cc1cc2[nH]ncc2c(-c2ncc3c(N4CCCC(C)(O)C4)nc(OCC45CCCC4N(C)CCC5)nc3c2F)c1Cl ; C#Cc1c(F)ccc2cc(O)cc(-c3ncc4c(N5CCOCC(O)(C6CC6)C5)nc(OCC56CCCC5N(C)CCC6)nc4c3F)c12] 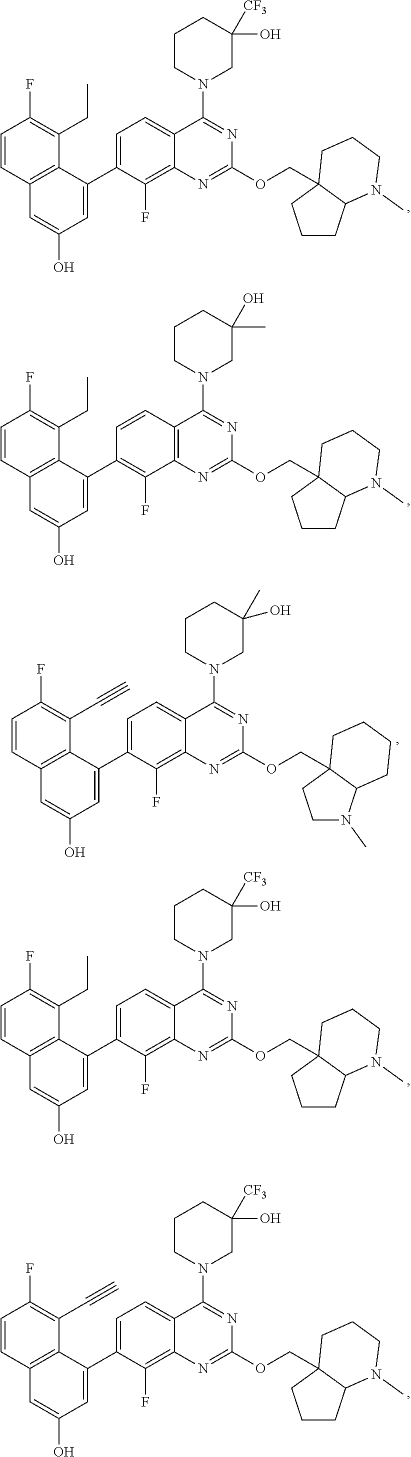 [SMILES: CCc1c(F)ccc2cc(O)cc(-c3ccc4c(N5CCCC(O)(C(F)(F)F)C5)nc(OCC56CCCC5N(C)CCC6)nc4c3F)c12 ; CCc1c(F)ccc2cc(O)cc(-c3ccc4c(N5CCCC(C)(O)C5)nc(OCC56CCCC5N(C)CCC6)nc4c3F)c12 ; C#Cc1c(F)ccc2cc(O)cc(-c3ccc4c(N5CCCC(C)(O)C5)nc(OCC56CCCCC5N(C)CC6)nc4c3F)c12 ; CCc1c(F)ccc2cc(O)cc(-c3ccc4c(N5CCCC(O)(C(F)(F)F)C5)nc(OCC56CCCC5N(C)CCC6)nc4c3F)c12 ; C#Cc1c(F)ccc2cc(O)cc(-c3ccc4c(N5CCCC(O)(C(F)(F)F)C5)nc(OCC56CCCC5N(C)CCC6)nc4c3F)c12] 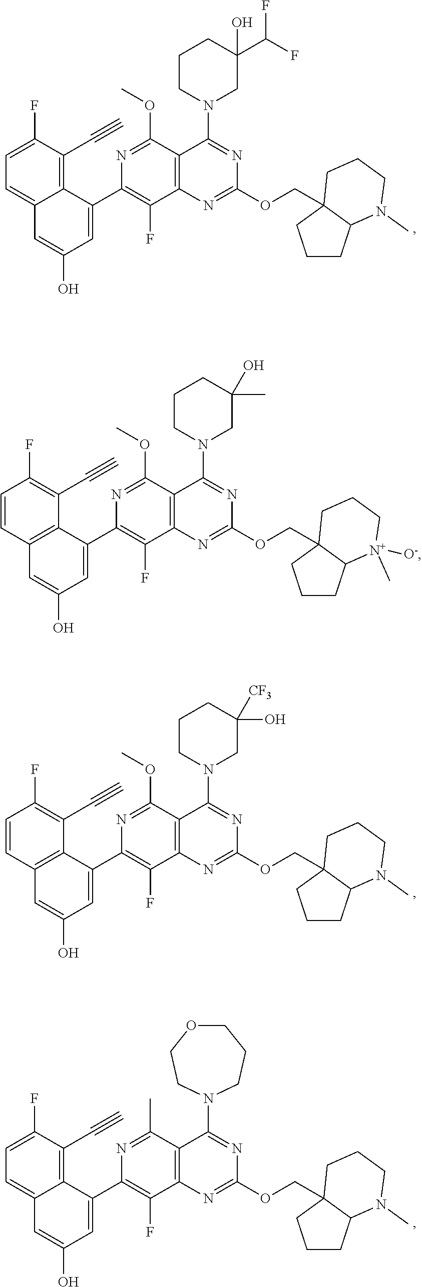 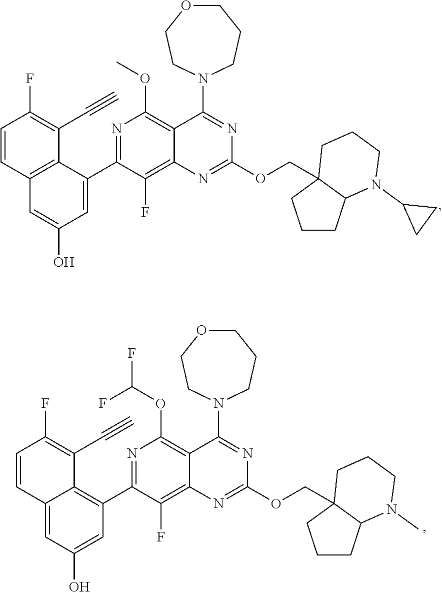 [SMILES: C#Cc1c(F)ccc2cc(O)cc(-c3nc(OC)c4c(N5CCCOCC5)nc(OCC56CCCC5N(C5CC5)CCC6)nc4c3F)c12 ; C#Cc1c(F)ccc2cc(O)cc(-c3nc(OC(F)F)c4c(N5CCCOCC5)nc(OCC56CCCC5N(C)CCC6)nc4c3F)c12] 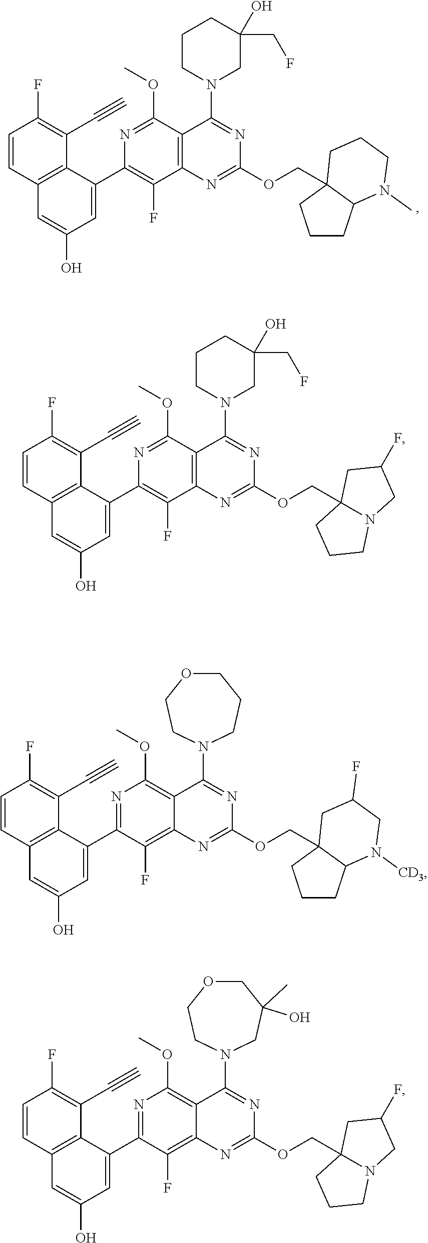 [SMILES: C#Cc1c(F)ccc2cc(O)cc(-c3nc(OC)c4c(N5CCCC(O)(CF)C5)nc(OCC56CCCC5N(C)CCC6)nc4c3F)c12 ; C#Cc1c(F)ccc2cc(O)cc(-c3nc(OC)c4c(N5CCCC(O)(CF)C5)nc(OCC56CCCN5CC(F)C6)nc4c3F)c12 ; [2H]C([2H])([2H])N1CC(F)CC2(COc3nc(N4CCCOCC4)c4c(OC)nc(-c5cc(O)cc6ccc(F)c(C#C)c56)c(F)c4n3)CCCC12 ; C#Cc1c(F)ccc2cc(O)cc(-c3nc(OC)c4c(N5CCOCC(C)(O)C5)nc(OCC56CCCN5CC(F)C6)nc4c3F)c12] 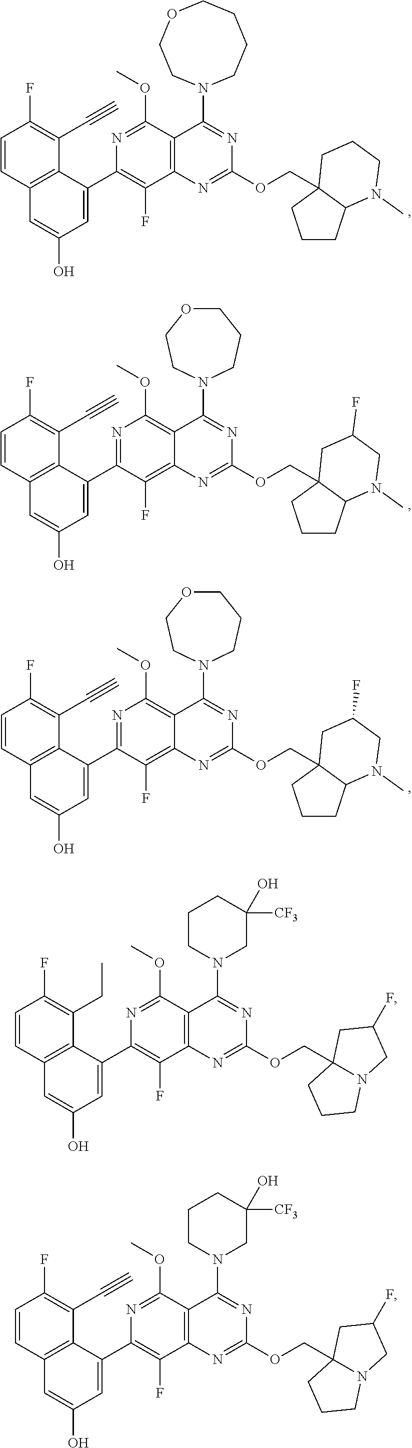 [SMILES: C#Cc1c(F)ccc2cc(O)cc(-c3nc(OC)c4c(N5CCCCOCC5)nc(OCC56CCCC5N(C)CCC6)nc4c3F)c12 ; C#Cc1c(F)ccc2cc(O)cc(-c3nc(OC)c4c(N5CCCOCC5)nc(OCC56CCCC5N(C)CC(F)C6)nc4c3F)c12 ; C#Cc1c(F)ccc2cc(O)cc(-c3nc(OC)c4c(N5CCCOCC5)nc(OCC56CCCC5N(C)C[C@@H](F)C6)nc4c3F)c12 ; CCc1c(F)ccc2cc(O)cc(-c3nc(OC)c4c(N5CCCC(O)(C(F)(F)F)C5)nc(OCC56CCCN5CC(F)C6)nc4c3F)c12 ; C#Cc1c(F)ccc2cc(O)cc(-c3nc(OC)c4c(N5CCCC(O)(C(F)(F)F)C5)nc(OCC56CCCN5CC(F)C6)nc4c3F)c12] 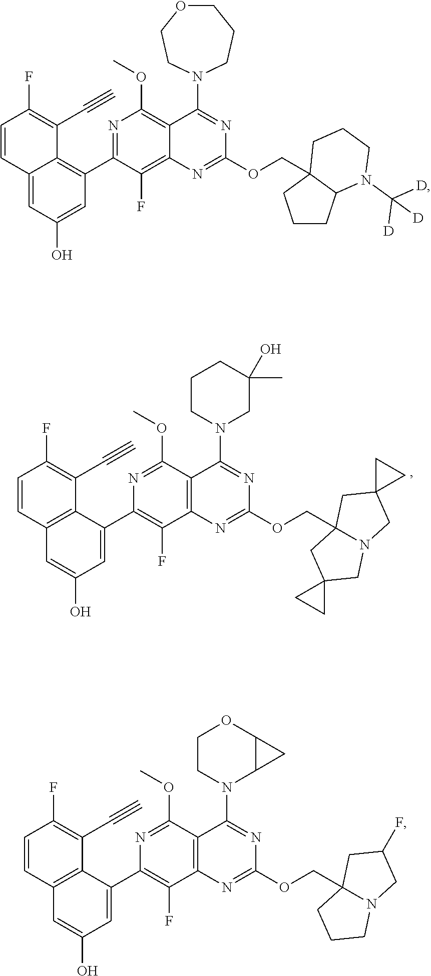 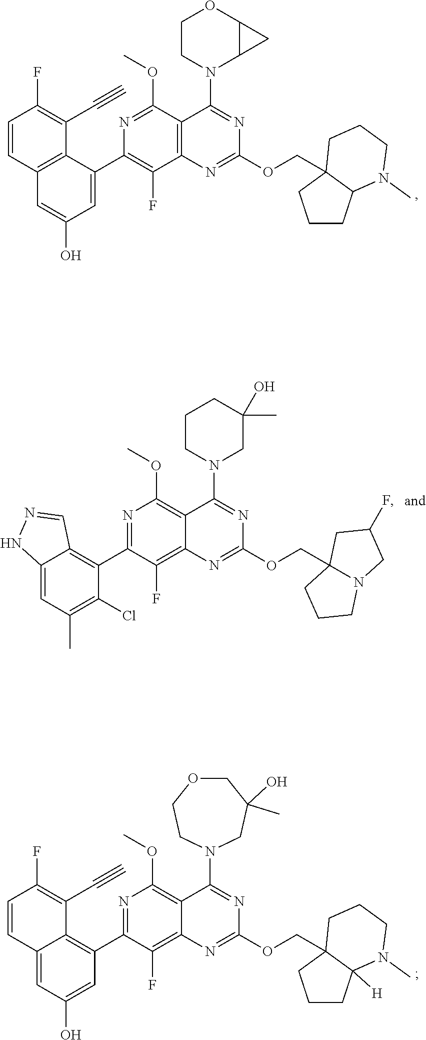 [SMILES: C#Cc1c(F)ccc2cc(O)cc(-c3nc(OC)c4c(N5CCOC6CC65)nc(OCC56CCCC5N(C)CCC6)nc4c3F)c12 ; COc1nc(-c2c(Cl)c(C)cc3[nH]ncc23)c(F)c2nc(OCC34CCCN3CC(F)C4)nc(N3CCCC(C)(O)C3)c12 ; "and" ; C#Cc1c(F)ccc2cc(O)cc(-c3nc(OC)c4c(N5CCOCC(C)(O)C5)nc(OCC56CCCC5N(C)CCC6)nc4c3F)c12]
or a pharmaceutically acceptable salt thereof.

41. The compound of claim 40 that is selected from the group consisting of: 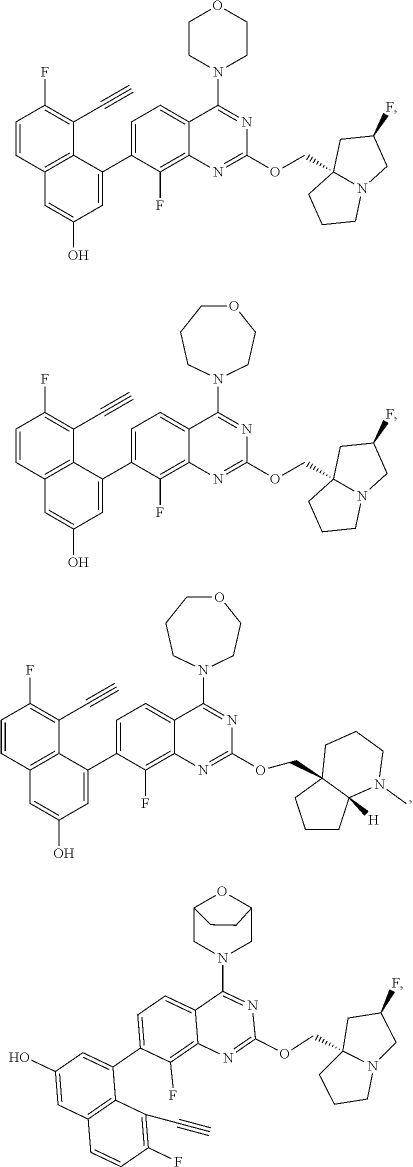 [SMILES: C#Cc1c(F)ccc2cc(O)cc(-c3ccc4c(N5CCOCC5)nc(OC[C@@]56CCCN5C[C@H](F)C6)nc4c3F)c12 ; C#Cc1c(F)ccc2cc(O)cc(-c3ccc4c(N5CCCOCC5)nc(OC[C@@]56CCCN5C[C@H](F)C6)nc4c3F)c12 ; C#Cc1c(F)ccc2cc(O)cc(-c3ccc4c(N5CCCOCC5)nc(OC[C@]56CCC[C@H]5N(C)CCC6)nc4c3F)c12 ; C#Cc1c(F)ccc2cc(O)cc(-c3ccc4c(N5CC6CCC(C5)O6)nc(OC[C@@]56CCCN5C[C@H](F)C6)nc4c3F)c12] 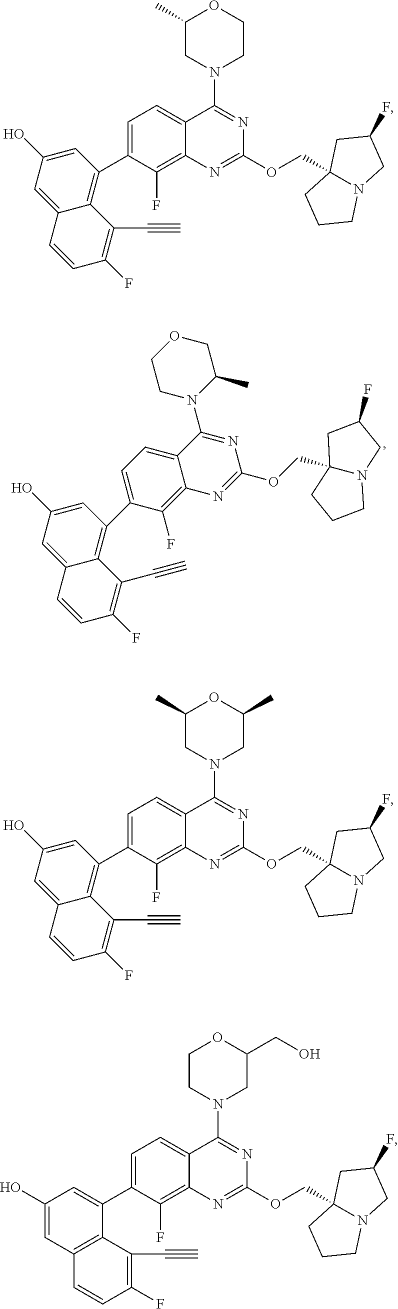 [SMILES: C#Cc1c(F)ccc2cc(O)cc(-c3ccc4c(N5CCO[C@@H](C)C5)nc(OC[C@@]56CCCN5C[C@H](F)C6)nc4c3F)c12 ; C#Cc1c(F)ccc2cc(O)cc(-c3ccc4c(N5CCOC[C@H]5C)nc(OC[C@@]56CCCN5C[C@H](F)C6)nc4c3F)c12 ; C#Cc1c(F)ccc2cc(O)cc(-c3ccc4c(N5C[C@@H](C)O[C@@H](C)C5)nc(OC[C@@]56CCCN5C[C@H](F)C6)nc4c3F)c12 ; C#Cc1c(F)ccc2cc(O)cc(-c3ccc4c(N5CCOC(CO)C5)nc(OC[C@@]56CCCN5C[C@H](F)C6)nc4c3F)c12] 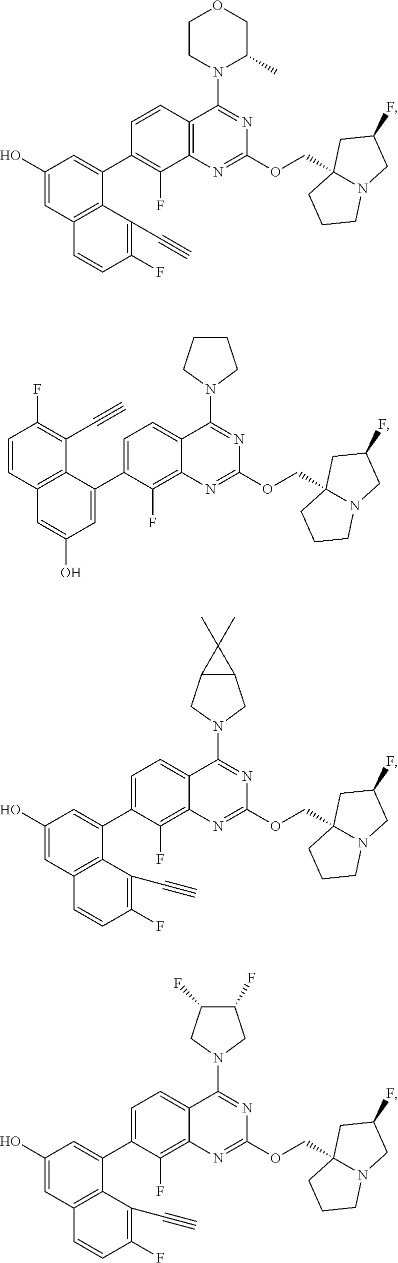 [SMILES: C#Cc1c(F)ccc2cc(O)cc(-c3ccc4c(N5CCOC[C@@H]5C)nc(OC[C@@]56CCCN5C[C@H](F)C6)nc4c3F)c12 ; C#Cc1c(F)ccc2cc(O)cc(-c3ccc4c(N5CCCC5)nc(OC[C@@]56CCCN5C[C@H](F)C6)nc4c3F)c12 ; C#Cc1c(F)ccc2cc(O)cc(-c3ccc4c(N5CC6C(C5)C6(C)C)nc(OC[C@@]56CCCN5C[C@H](F)C6)nc4c3F)c12 ; C#Cc1c(F)ccc2cc(O)cc(-c3ccc4c(N5C[C@@H](F)[C@@H](F)C5)nc(OC[C@@]56CCCN5C[C@H](F)C6)nc4c3F)c12] 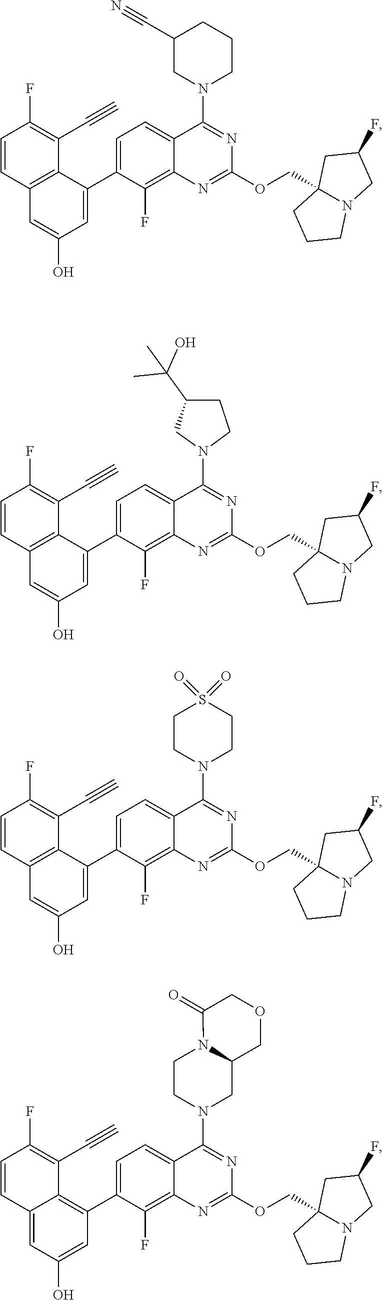 [SMILES: C#Cc1c(F)ccc2cc(O)cc(-c3ccc4c(N5CCCC(C#N)C5)nc(OC[C@@]56CCCN5C[C@H](F)C6)nc4c3F)c12 ; C#Cc1c(F)ccc2cc(O)cc(-c3ccc4c(N5CC[C@@H](C(C)(C)O)C5)nc(OC[C@@]56CCCN5C[C@H](F)C6)nc4c3F)c12 ; C#Cc1c(F)ccc2cc(O)cc(-c3ccc4c(N5CCS(=O)(=O)CC5)nc(OC[C@@]56CCCN5C[C@H](F)C6)nc4c3F)c12 ; C#Cc1c(F)ccc2cc(O)cc(-c3ccc4c(N5CCN6C(=O)COC[C@H]6C5)nc(OC[C@@]56CCCN5C[C@H](F)C6)nc4c3F)c12] 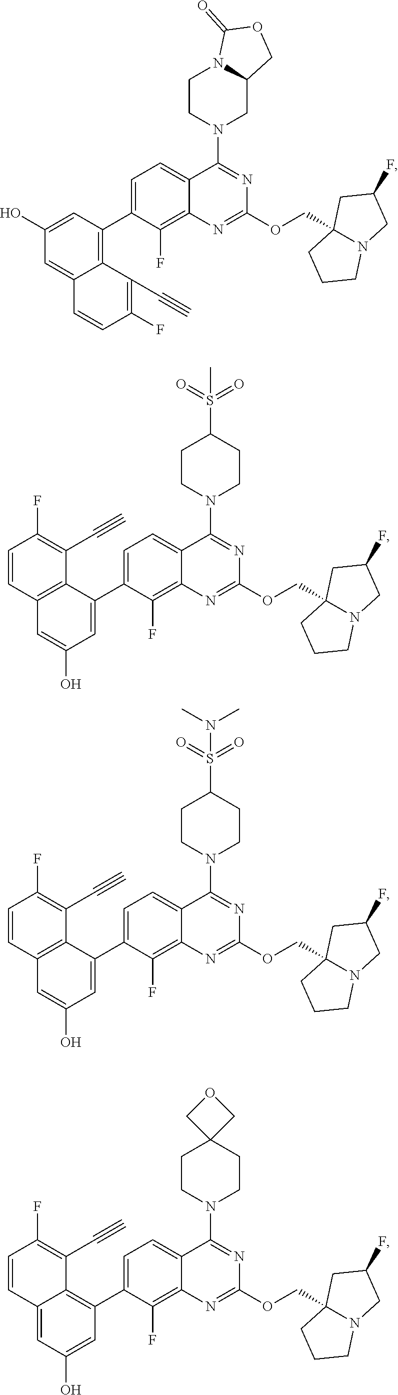 [SMILES: C#Cc1c(F)ccc2cc(O)cc(-c3ccc4c(N5CCN6C(=O)OC[C@H]6C5)nc(OC[C@@]56CCCN5C[C@H](F)C6)nc4c3F)c12 ; C#Cc1c(F)ccc2cc(O)cc(-c3ccc4c(N5CCC(S(C)(=O)=O)CC5)nc(OC[C@@]56CCCN5C[C@H](F)C6)nc4c3F)c12 ; C#Cc1c(F)ccc2cc(O)cc(-c3ccc4c(N5CCC(S(=O)(=O)N(C)C)CC5)nc(OC[C@@]56CCCN5C[C@H](F)C6)nc4c3F)c12 ; C#Cc1c(F)ccc2cc(O)cc(-c3ccc4c(N5CCC6(CC5)COC6)nc(OC[C@@]56CCCN5C[C@H](F)C6)nc4c3F)c12] 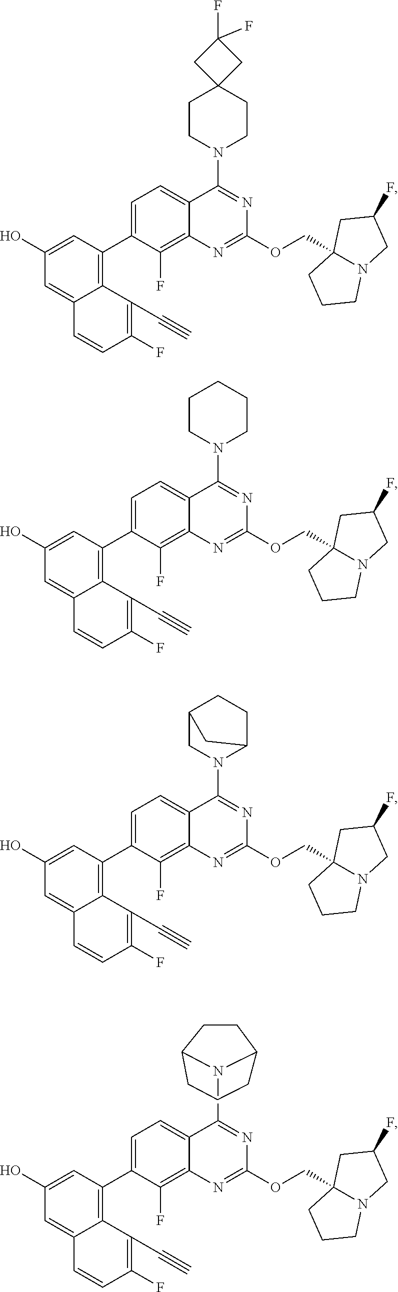 [SMILES: C#Cc1c(F)ccc2cc(O)cc(-c3ccc4c(N5CCC6(CC5)CC(F)(F)C6)nc(OC[C@@]56CCCN5C[C@H](F)C6)nc4c3F)c12 ; C#Cc1c(F)ccc2cc(O)cc(-c3ccc4c(N5CCCCC5)nc(OC[C@@]56CCCN5C[C@H](F)C6)nc4c3F)c12 ; C#Cc1c(F)ccc2cc(O)cc(-c3ccc4c(N5CC6CCC5C6)nc(OC[C@@]56CCCN5C[C@H](F)C6)nc4c3F)c12 ; C#Cc1c(F)ccc2cc(O)cc(-c3ccc4c(N5C6CCCC5CC6)nc(OC[C@@]56CCCN5C[C@H](F)C6)nc4c3F)c12] 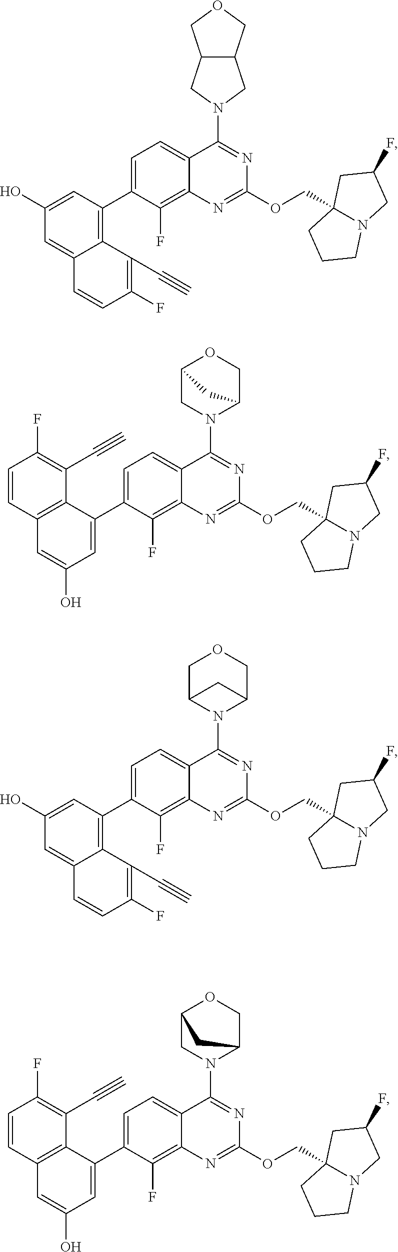 [SMILES: C#Cc1c(F)ccc2cc(O)cc(-c3ccc4c(N5CC6COCC6C5)nc(OC[C@@]56CCCN5C[C@H](F)C6)nc4c3F)c12 ; C#Cc1c(F)ccc2cc(O)cc(-c3ccc4c(N5C[C@@H]6C[C@H]5CO6)nc(OC[C@@]56CCCN5C[C@H](F)C6)nc4c3F)c12 ; C#Cc1c(F)ccc2cc(O)cc(-c3ccc4c(N5C6COCC5C6)nc(OC[C@@]56CCCN5C[C@H](F)C6)nc4c3F)c12 ; C#Cc1c(F)ccc2cc(O)cc(-c3ccc4c(N5C[C@H]6C[C@@H]5CO6)nc(OC[C@@]56CCCN5C[C@H](F)C6)nc4c3F)c12] 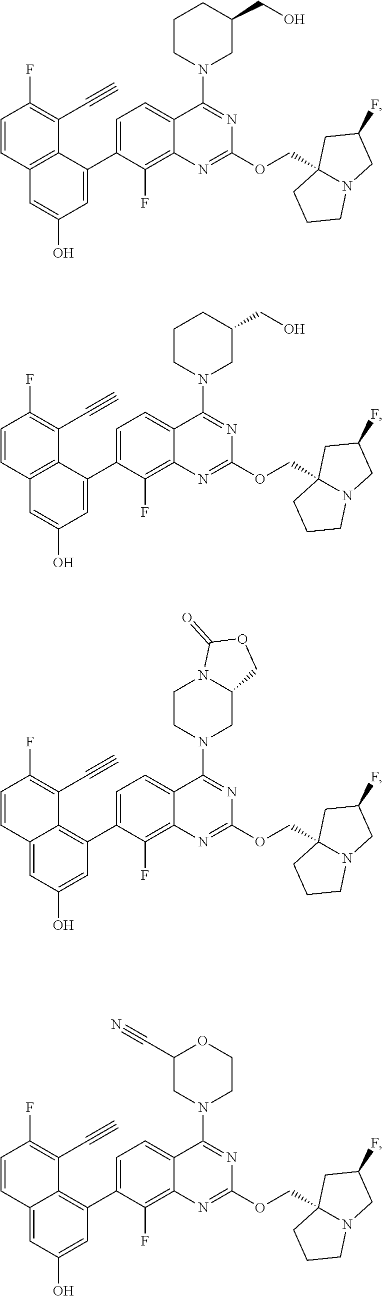 [SMILES: C#Cc1c(F)ccc2cc(O)cc(-c3ccc4c(N5CCC[C@@H](CO)C5)nc(OC[C@@]56CCCN5C[C@H](F)C6)nc4c3F)c12 ; C#Cc1c(F)ccc2cc(O)cc(-c3ccc4c(N5CCC[C@H](CO)C5)nc(OC[C@@]56CCCN5C[C@H](F)C6)nc4c3F)c12 ; C#Cc1c(F)ccc2cc(O)cc(-c3ccc4c(N5CCN6C(=O)OC[C@@H]6C5)nc(OC[C@@]56CCCN5C[C@H](F)C6)nc4c3F)c12 ; C#Cc1c(F)ccc2cc(O)cc(-c3ccc4c(N5CCOC(C#N)C5)nc(OC[C@@]56CCCN5C[C@H](F)C6)nc4c3F)c12] 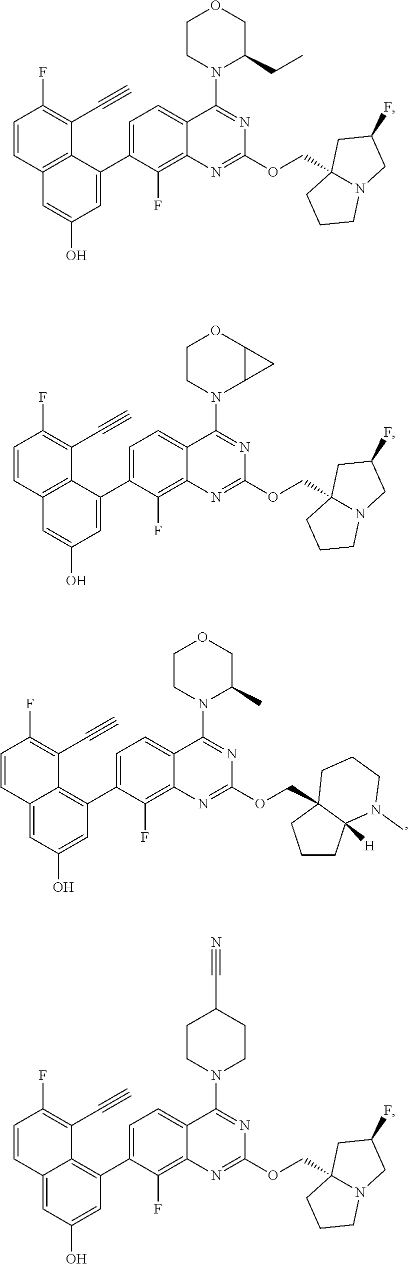 [SMILES: C#Cc1c(F)ccc2cc(O)cc(-c3ccc4c(N5CCOC[C@H]5CC)nc(OC[C@@]56CCCN5C[C@H](F)C6)nc4c3F)c12 ; C#Cc1c(F)ccc2cc(O)cc(-c3ccc4c(N5CCOC6CC65)nc(OC[C@@]56CCCN5C[C@H](F)C6)nc4c3F)c12 ; C#Cc1c(F)ccc2cc(O)cc(-c3ccc4c(N5CCOC[C@H]5C)nc(OC[C@]56CCC[C@H]5N(C)CCC6)nc4c3F)c12 ; C#Cc1c(F)ccc2cc(O)cc(-c3ccc4c(N5CCC(C#N)CC5)nc(OC[C@@]56CCCN5C[C@H](F)C6)nc4c3F)c12] 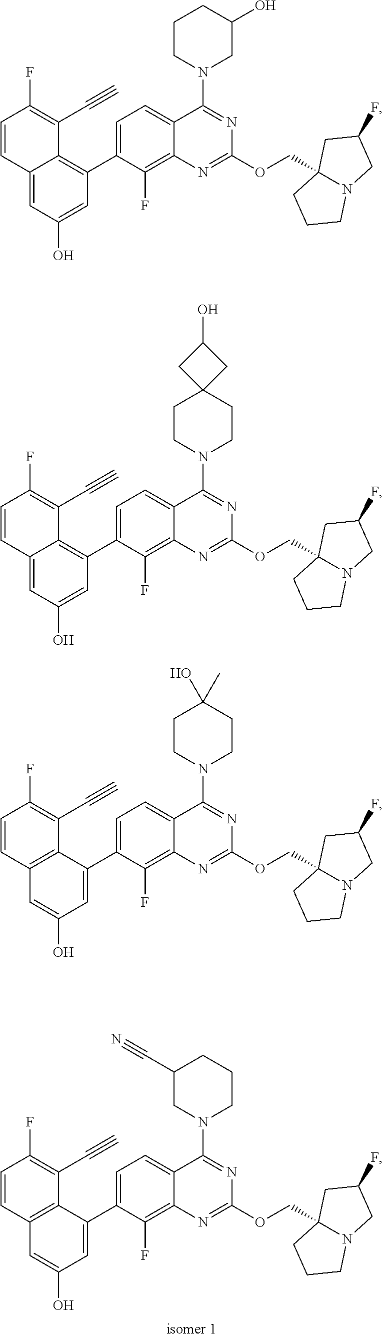 [SMILES: C#Cc1c(F)ccc2cc(O)cc(-c3ccc4c(N5CCCC(O)C5)nc(OC[C@@]56CCCN5C[C@H](F)C6)nc4c3F)c12 ; C#Cc1c(F)ccc2cc(O)cc(-c3ccc4c(N5CCC6(CC5)CC(O)C6)nc(OC[C@@]56CCCN5C[C@H](F)C6)nc4c3F)c12 ; C#Cc1c(F)ccc2cc(O)cc(-c3ccc4c(N5CCC(C)(O)CC5)nc(OC[C@@]56CCCN5C[C@H](F)C6)nc4c3F)c12 ; C#Cc1c(F)ccc2cc(O)cc(-c3ccc4c(N5CCCC(C#N)C5)nc(OC[C@@]56CCCN5C[C@H](F)C6)nc4c3F)c12] 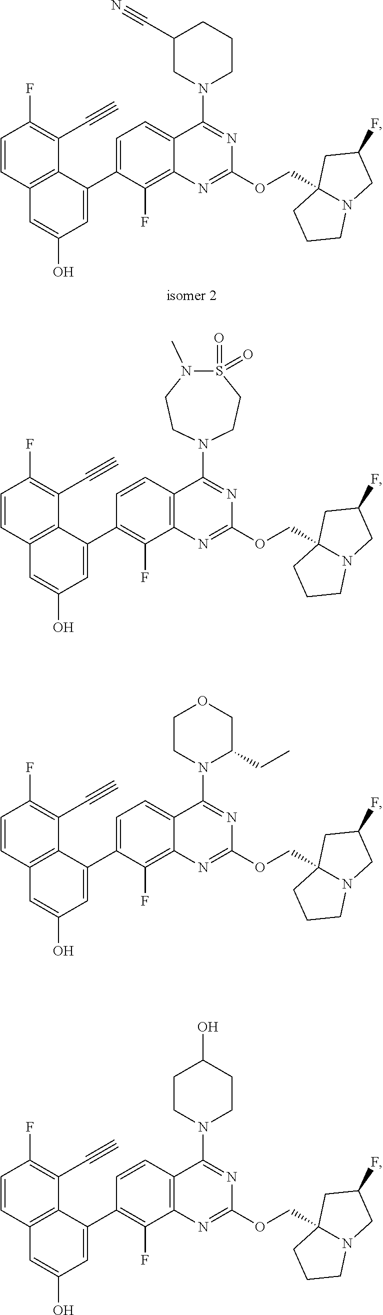 [SMILES: C#Cc1c(F)ccc2cc(O)cc(-c3ccc4c(N5CCCC(C#N)C5)nc(OC[C@@]56CCCN5C[C@H](F)C6)nc4c3F)c12 ; C#Cc1c(F)ccc2cc(O)cc(-c3ccc4c(N5CCN(C)S(=O)(=O)CC5)nc(OC[C@@]56CCCN5C[C@H](F)C6)nc4c3F)c12 ; C#Cc1c(F)ccc2cc(O)cc(-c3ccc4c(N5CCOC[C@@H]5CC)nc(OC[C@@]56CCCN5C[C@H](F)C6)nc4c3F)c12 ; C#Cc1c(F)ccc2cc(O)cc(-c3ccc4c(N5CCC(O)CC5)nc(OC[C@@]56CCCN5C[C@H](F)C6)nc4c3F)c12] 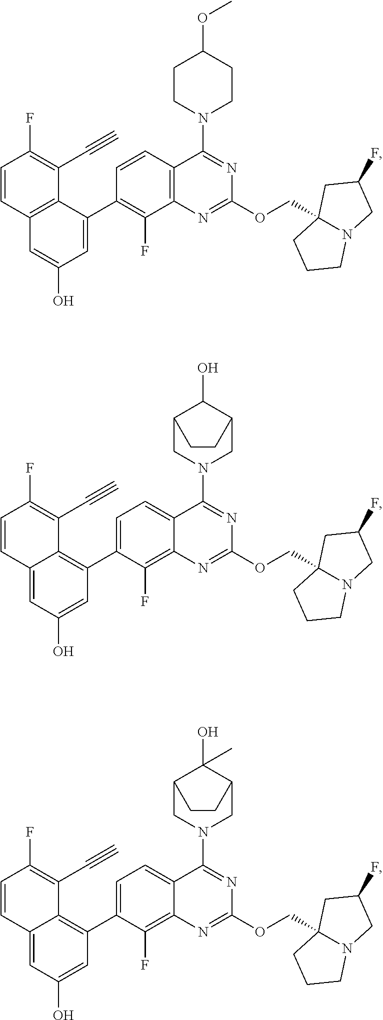 [SMILES: C#Cc1c(F)ccc2cc(O)cc(-c3ccc4c(N5CCC(OC)CC5)nc(OC[C@@]56CCCN5C[C@H](F)C6)nc4c3F)c12 ; C#Cc1c(F)ccc2cc(O)cc(-c3ccc4c(N5CC6CCC(C5)C6O)nc(OC[C@@]56CCCN5C[C@H](F)C6)nc4c3F)c12 ; C#Cc1c(F)ccc2cc(O)cc(-c3ccc4c(N5CC6CCC(C5)C6(C)O)nc(OC[C@@]56CCCN5C[C@H](F)C6)nc4c3F)c12] 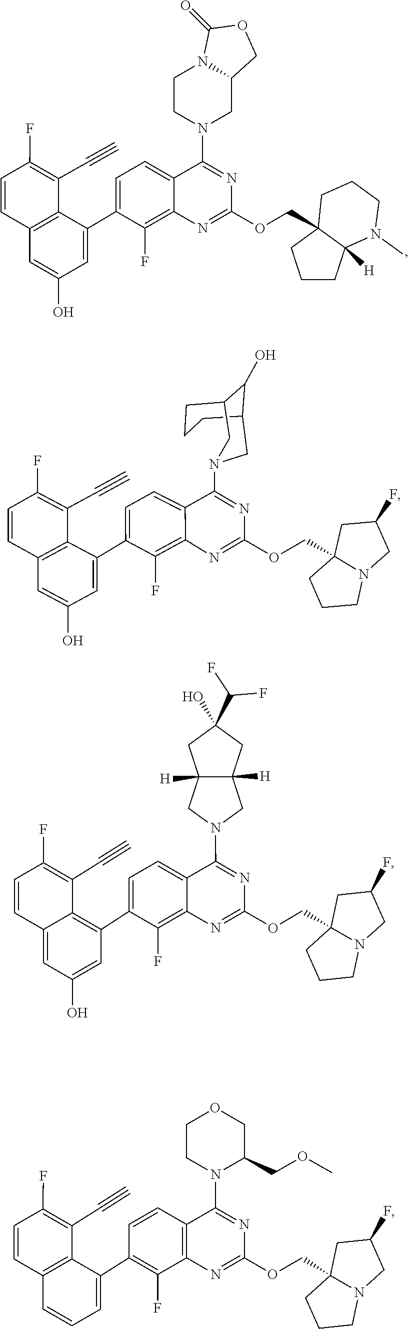 [SMILES: C#Cc1c(F)ccc2cc(O)cc(-c3ccc4c(N5CCN6C(=O)OC[C@@H]6C5)nc(OC[C@]56CCC[C@H]5N(C)CCC6)nc4c3F)c12 ; C#Cc1c(F)ccc2cc(O)cc(-c3ccc4c(N5CC6CCCC(C5)C6O)nc(OC[C@@]56CCCN5C[C@H](F)C6)nc4c3F)c12 ; C#Cc1c(F)ccc2cc(O)cc(-c3ccc4c(N5C[C@@H]6C[C@@](O)(C(F)F)C[C@@H]6C5)nc(OC[C@@]56CCCN5C[C@H](F)C6)nc4c3F)c12 ; C#Cc1c(F)ccc2cccc(-c3ccc4c(N5CCOC[C@H]5COC)nc(OC[C@@]56CCCN5C[C@H](F)C6)nc4c3F)c12] 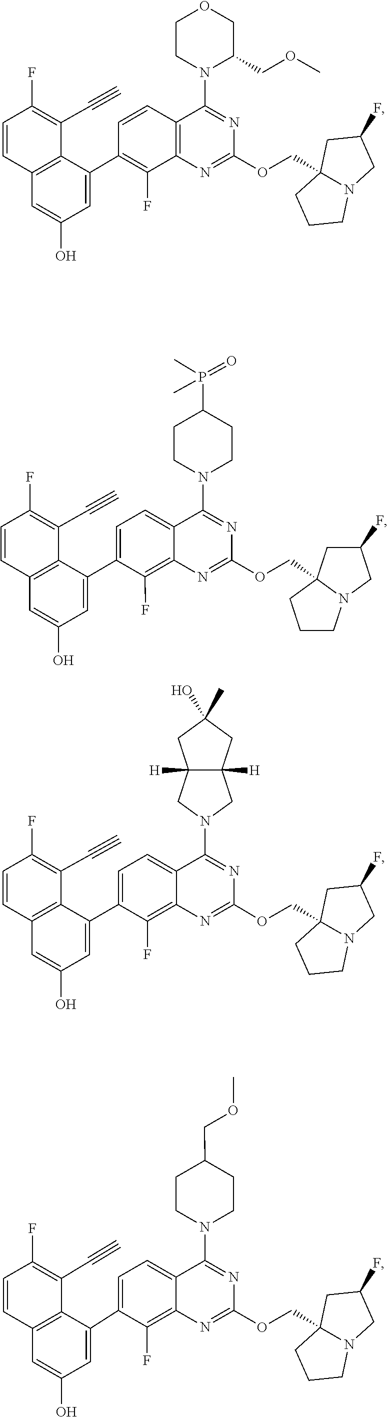 [SMILES: C#Cc1c(F)ccc2cc(O)cc(-c3ccc4c(N5CCOC[C@@H]5COC)nc(OC[C@@]56CCCN5C[C@H](F)C6)nc4c3F)c12 ; C#Cc1c(F)ccc2cc(O)cc(-c3ccc4c(N5CCC(P(C)(C)=O)CC5)nc(OC[C@@]56CCCN5C[C@H](F)C6)nc4c3F)c12 ; C#Cc1c(F)ccc2cc(O)cc(-c3ccc4c(N5C[C@@H]6C[C@](C)(O)C[C@@H]6C5)nc(OC[C@@]56CCCN5C[C@H](F)C6)nc4c3F)c12 ; C#Cc1c(F)ccc2cc(O)cc(-c3ccc4c(N5CCC(COC)CC5)nc(OC[C@@]56CCCN5C[C@H](F)C6)nc4c3F)c12] 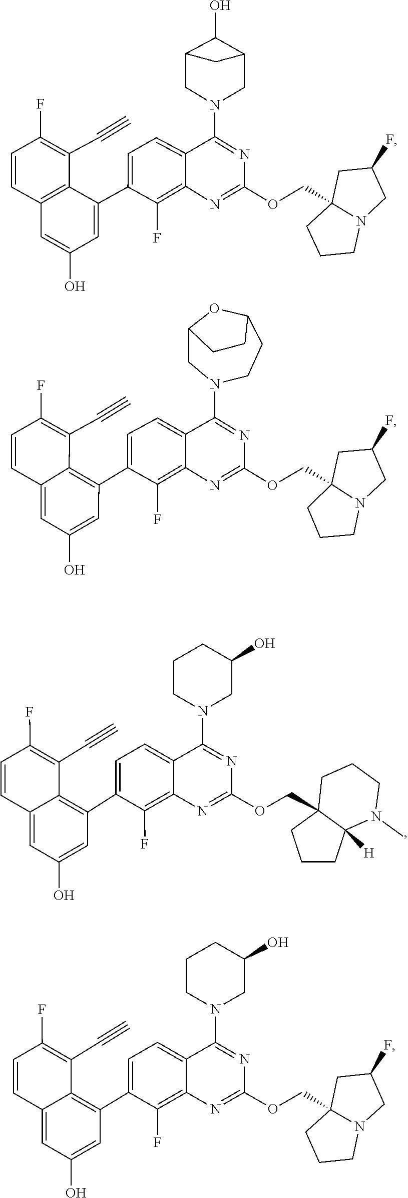 [SMILES: C#Cc1c(F)ccc2cc(O)cc(-c3ccc4c(N5CC6CC(C5)C6O)nc(OC[C@@]56CCCN5C[C@H](F)C6)nc4c3F)c12 ; C#Cc1c(F)ccc2cc(O)cc(-c3ccc4c(N5CCC6CCC(C5)O6)nc(OC[C@@]56CCCN5C[C@H](F)C6)nc4c3F)c12 ; C#Cc1c(F)ccc2cc(O)cc(-c3ccc4c(N5CCC[C@@H](O)C5)nc(OC[C@]56CCC[C@H]5N(C)CCC6)nc4c3F)c12 ; C#Cc1c(F)ccc2cc(O)cc(-c3ccc4c(N5CCC[C@@H](O)C5)nc(OC[C@@]56CCCN5C[C@H](F)C6)nc4c3F)c12] 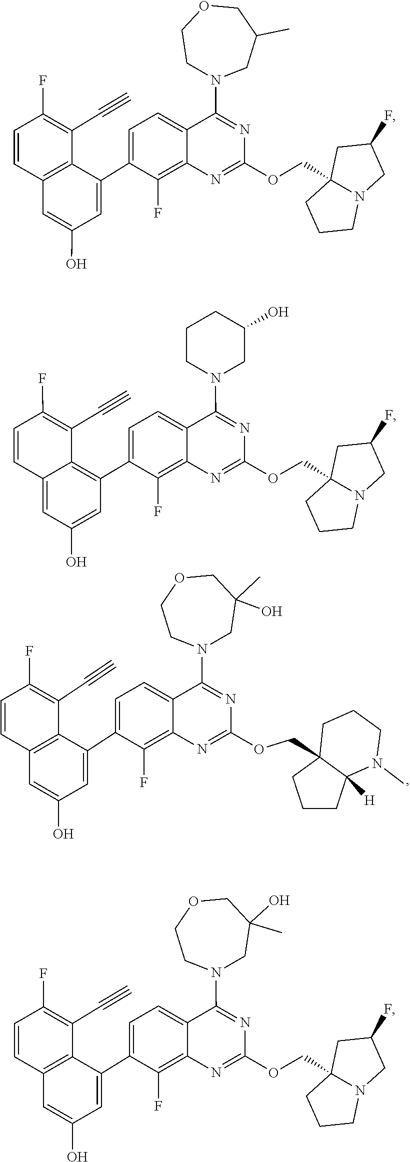 [SMILES: C#Cc1c(F)ccc2cc(O)cc(-c3ccc4c(N5CCOCC(C)C5)nc(OC[C@@]56CCCN5C[C@H](F)C6)nc4c3F)c12 ; C#Cc1c(F)ccc2cc(O)cc(-c3ccc4c(N5CCC[C@H](O)C5)nc(OC[C@@]56CCCN5C[C@H](F)C6)nc4c3F)c12 ; C#Cc1c(F)ccc2cc(O)cc(-c3ccc4c(N5CCOCC(C)(O)C5)nc(OC[C@]56CCC[C@H]5N(C)CCC6)nc4c3F)c12 ; C#Cc1c(F)ccc2cc(O)cc(-c3ccc4c(N5CCOCC(C)(O)C5)nc(OC[C@@]56CCCN5C[C@H](F)C6)nc4c3F)c12] 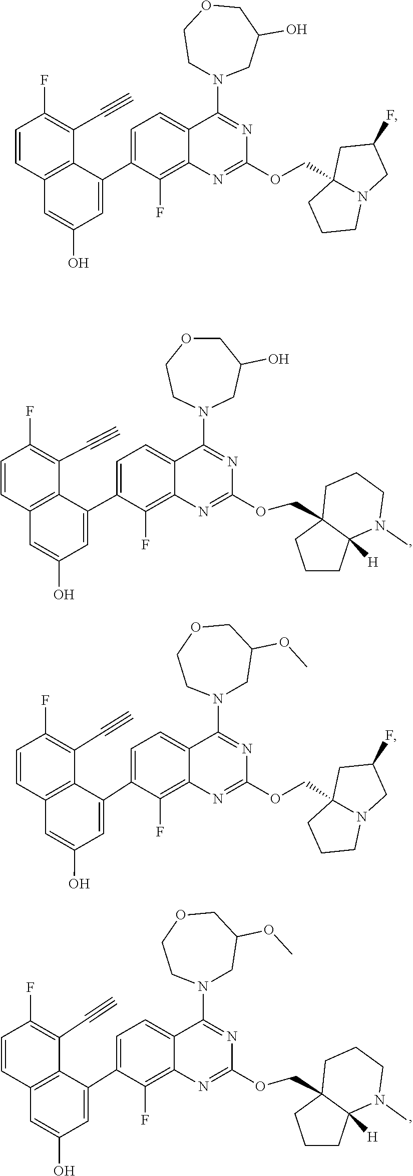 [SMILES: C#Cc1c(F)ccc2cc(O)cc(-c3ccc4c(N5CCOCC(O)C5)nc(OC[C@@]56CCCN5C[C@H](F)C6)nc4c3F)c12 ; C#Cc1c(F)ccc2cc(O)cc(-c3ccc4c(N5CCOCC(O)C5)nc(OC[C@]56CCC[C@H]5N(C)CCC6)nc4c3F)c12 ; C#Cc1c(F)ccc2cc(O)cc(-c3ccc4c(N5CCOCC(OC)C5)nc(OC[C@@]56CCCN5C[C@H](F)C6)nc4c3F)c12 ; C#Cc1c(F)ccc2cc(O)cc(-c3ccc4c(N5CCOCC(OC)C5)nc(OC[C@]56CCC[C@H]5N(C)CCC6)nc4c3F)c12] 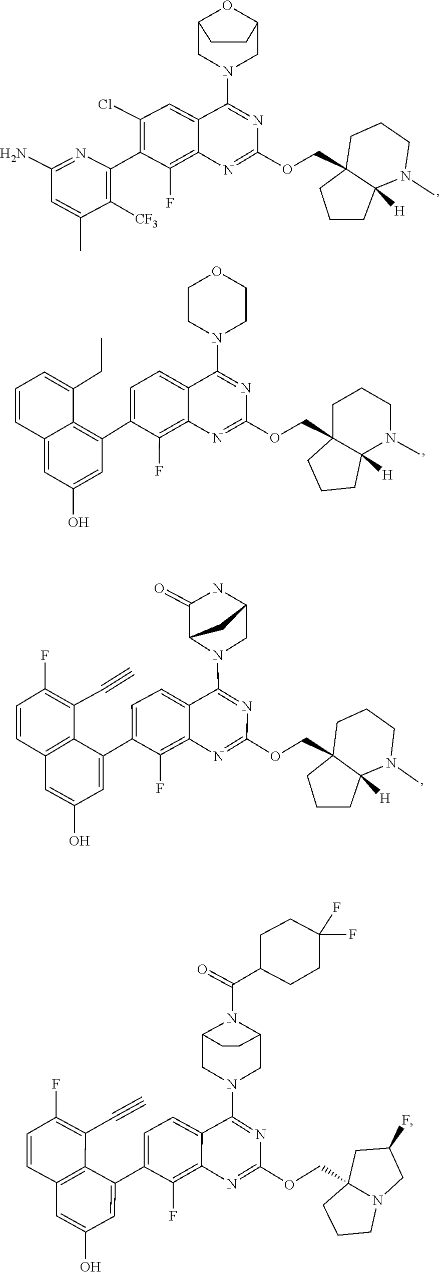 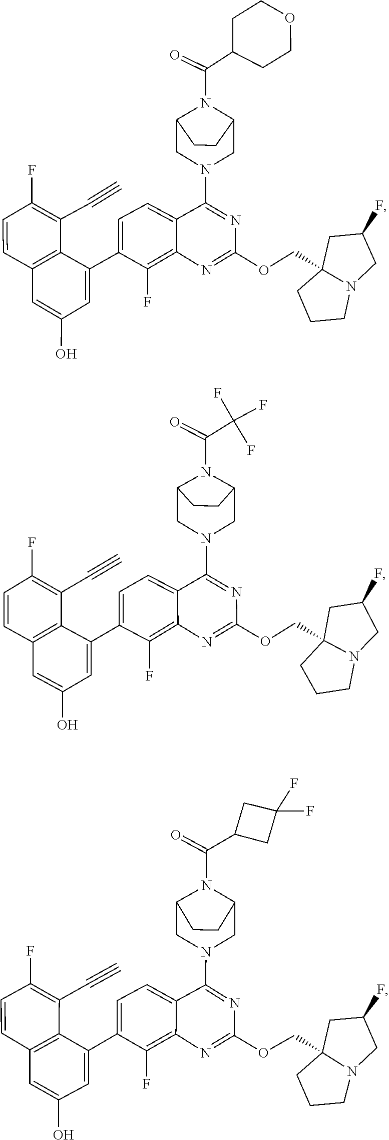 [SMILES: C#Cc1c(F)ccc2cc(O)cc(-c3ccc4c(N5CC6CCC(C5)N6C(=O)C5CCOCC5)nc(OC[C@@]56CCCN5C[C@H](F)C6)nc4c3F)c12 ; C#Cc1c(F)ccc2cc(O)cc(-c3ccc4c(N5CC6CCC(C5)N6C(=O)C(F)(F)F)nc(OC[C@@]56CCCN5C[C@H](F)C6)nc4c3F)c12 ; C#Cc1c(F)ccc2cc(O)cc(-c3ccc4c(N5CC6CCC(C5)N6C(=O)C5CC(F)(F)C5)nc(OC[C@@]56CCCN5C[C@H](F)C6)nc4c3F)c12] 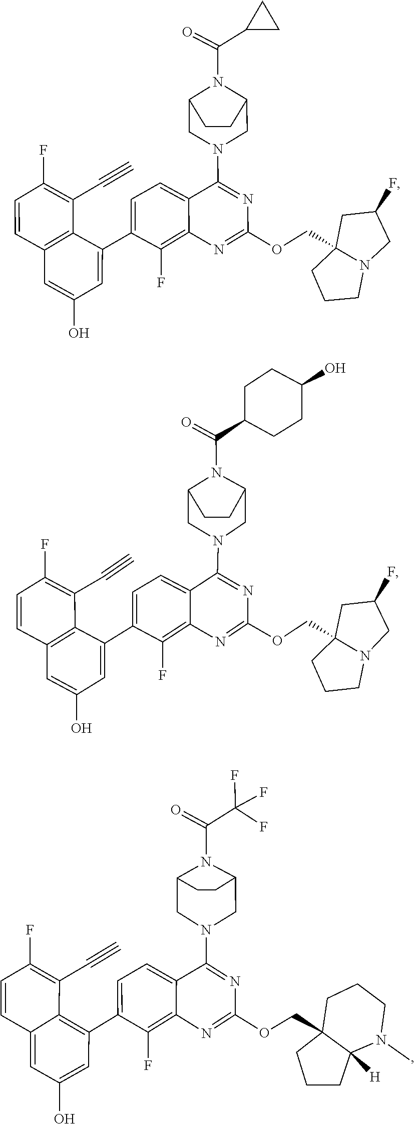 [SMILES: C#Cc1c(F)ccc2cc(O)cc(-c3ccc4c(N5CC6CCC(C5)N6C(=O)C5CC5)nc(OC[C@@]56CCCN5C[C@H](F)C6)nc4c3F)c12 ; C#Cc1c(F)ccc2cc(O)cc(-c3ccc4c(N5CC6CCC(C5)N6C(=O)[C@H]5CC[C@@H](O)CC5)nc(OC[C@@]56CCCN5C[C@H](F)C6)nc4c3F)c12 ; C#Cc1c(F)ccc2cc(O)cc(-c3ccc4c(N5CC6CCC(C5)N6C(=O)C(F)(F)F)nc(OC[C@]56CCC[C@H]5N(C)CCC6)nc4c3F)c12] 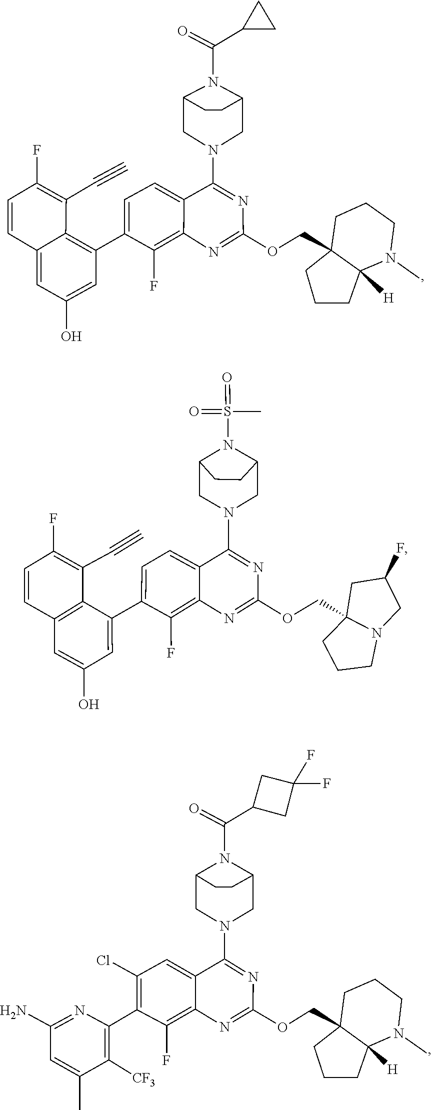 [SMILES: C#Cc1c(F)ccc2cc(O)cc(-c3ccc4c(N5CC6CCC(C5)N6C(=O)C5CC5)nc(OC[C@]56CCC[C@H]5N(C)CCC6)nc4c3F)c12 ; C#Cc1c(F)ccc2cc(O)cc(-c3ccc4c(N5CC6CCC(C5)N6S(C)(=O)=O)nc(OC[C@@]56CCCN5C[C@H](F)C6)nc4c3F)c12 ; Cc1cc(N)nc(-c2c(Cl)cc3c(N4CC5CCC(C4)N5C(=O)C4CC(F)(F)C4)nc(OC[C@]45CCC[C@H]4N(C)CCC5)nc3c2F)c1C(F)(F)F] 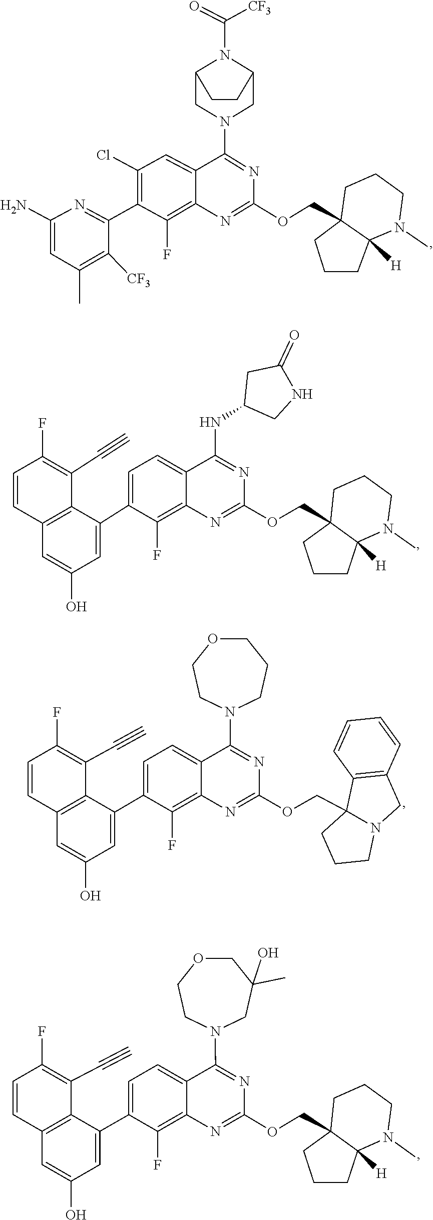 [SMILES: Cc1cc(N)nc(-c2c(Cl)cc3c(N4CC5CCC(C4)N5C(=O)C(F)(F)F)nc(OC[C@]45CCC[C@H]4N(C)CCC5)nc3c2F)c1C(F)(F)F ; C#Cc1c(F)ccc2cc(O)cc(-c3ccc4c(N[C@H]5CNC(=O)C5)nc(OC[C@]56CCC[C@H]5N(C)CCC6)nc4c3F)c12 ; C#Cc1c(F)ccc2cc(O)cc(-c3ccc4c(N5CCCOCC5)nc(OCC56CCCN5Cc5ccccc56)nc4c3F)c12 ; C#Cc1c(F)ccc2cc(O)cc(-c3ccc4c(N5CCOCC(C)(O)C5)nc(OC[C@]56CCC[C@H]5N(C)CCC6)nc4c3F)c12] 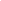 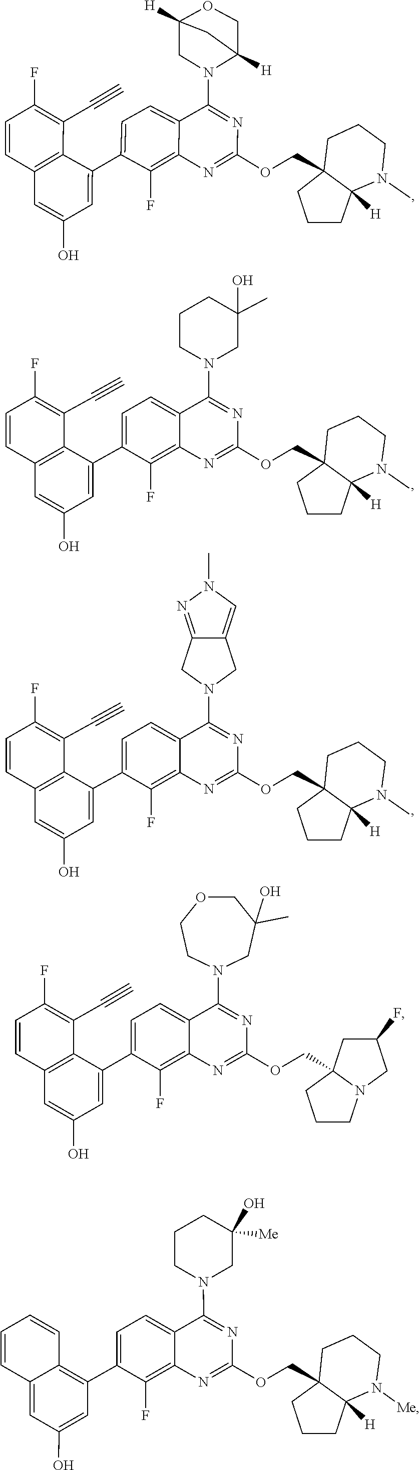 [SMILES: C#Cc1c(F)ccc2cc(O)cc(-c3ccc4c(N5C[C@H]6C[C@@H]5CO6)nc(OC[C@]56CCC[C@H]5N(C)CCC6)nc4c3F)c12 ; C#Cc1c(F)ccc2cc(O)cc(-c3ccc4c(N5CCCC(C)(O)C5)nc(OC[C@]56CCC[C@H]5N(C)CCC6)nc4c3F)c12 ; C#Cc1c(F)ccc2cc(O)cc(-c3ccc4c(N5Cc6cn(C)nc6C5)nc(OC[C@]56CCC[C@H]5N(C)CCC6)nc4c3F)c12 ; C#Cc1c(F)ccc2cc(O)cc(-c3ccc4c(N5CCOCC(C)(O)C5)nc(OC[C@@]56CCCN5C[C@H](F)C6)nc4c3F)c12 ; CN1CCC[C@@]2(COc3nc(N4CCC[C@@](C)(O)C4)c4ccc(-c5cc(O)cc6ccccc56)c(F)c4n3)CCC[C@@H]12] 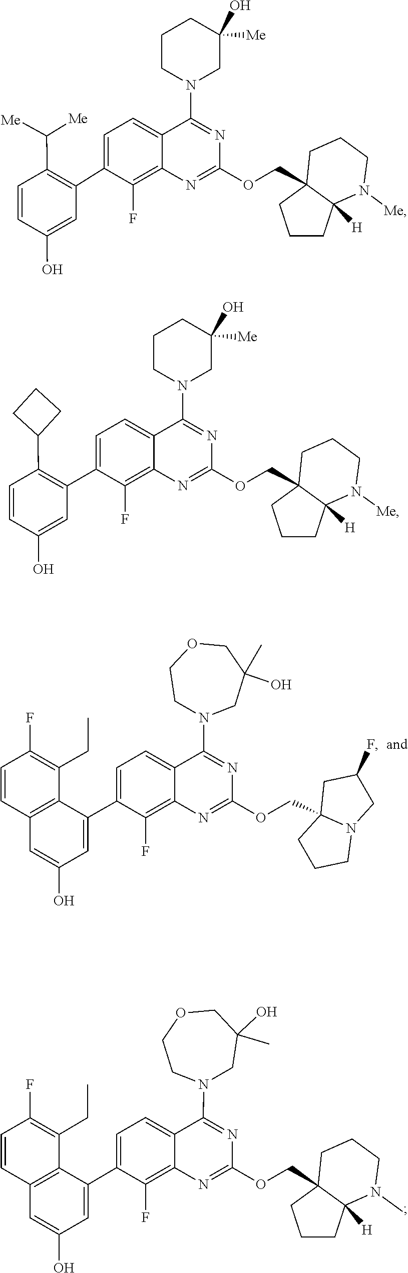 [SMILES: CC(C)c1ccc(O)cc1-c1ccc2c(N3CCC[C@@](C)(O)C3)nc(OC[C@]34CCC[C@H]3N(C)CCC4)nc2c1F ; CN1CCC[C@@]2(COc3nc(N4CCC[C@@](C)(O)C4)c4ccc(-c5cc(O)ccc5C5CCC5)c(F)c4n3)CCC[C@@H]12 ; CCc1c(F)ccc2cc(O)cc(-c3ccc4c(N5CCOCC(C)(O)C5)nc(OC[C@@]56CCCN5C[C@H](F)C6)nc4c3F)c12 ; CCc1c(F)ccc2cc(O)cc(-c3ccc4c(N5CCOCC(C)(O)C5)nc(OC[C@]56CCC[C@H]5N(C)CCC6)nc4c3F)c12]
or a pharmaceutically acceptable salt thereof.

42. The compound of claim 4 that is selected from the group consisting of: 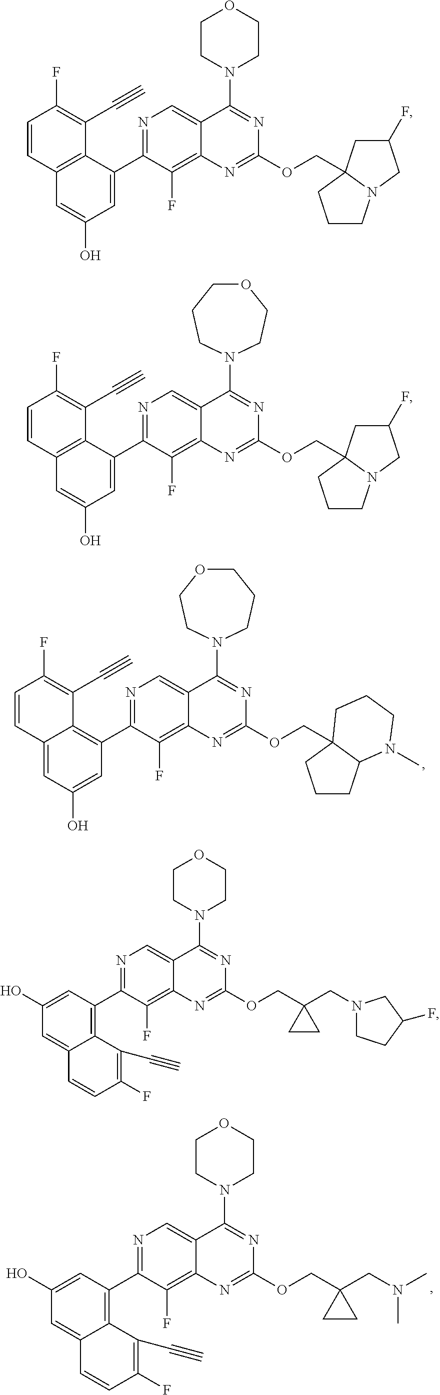 [SMILES: C#Cc1c(F)ccc2cc(O)cc(-c3ncc4c(N5CCOCC5)nc(OCC56CCCN5CC(F)C6)nc4c3F)c12 ; C#Cc1c(F)ccc2cc(O)cc(-c3ncc4c(N5CCCOCC5)nc(OCC56CCCN5CC(F)C6)nc4c3F)c12 ; C#Cc1c(F)ccc2cc(O)cc(-c3ncc4c(N5CCCOCC5)nc(OCC56CCCC5N(C)CCC6)nc4c3F)c12 ; C#Cc1c(F)ccc2cc(O)cc(-c3ncc4c(N5CCOCC5)nc(OCC5(CN6CCC(F)C6)CC5)nc4c3F)c12 ; C#Cc1c(F)ccc2cc(O)cc(-c3ncc4c(N5CCOCC5)nc(OCC5(CN(C)C)CC5)nc4c3F)c12] 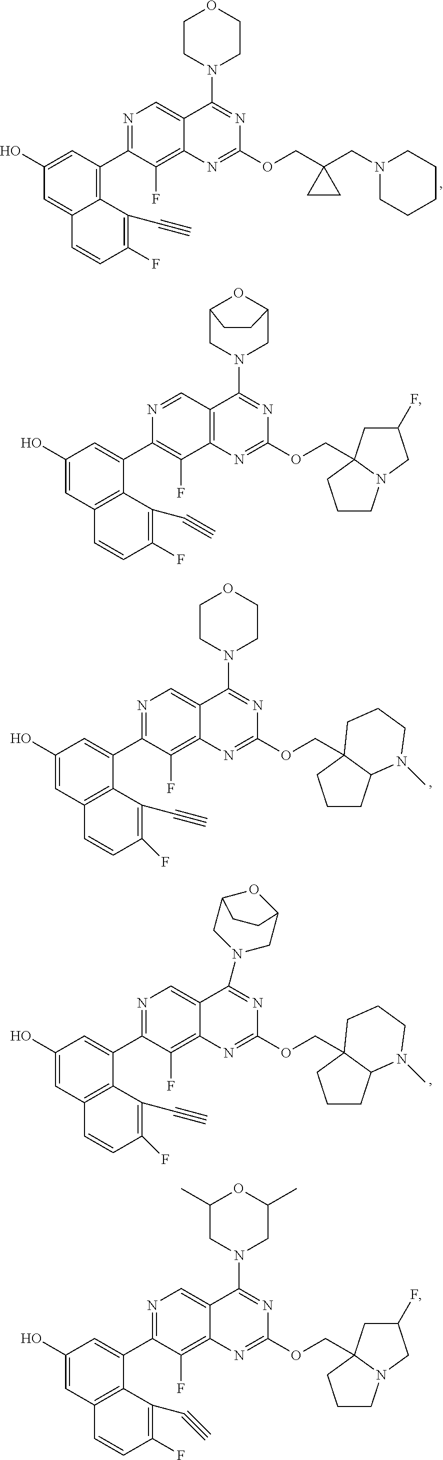 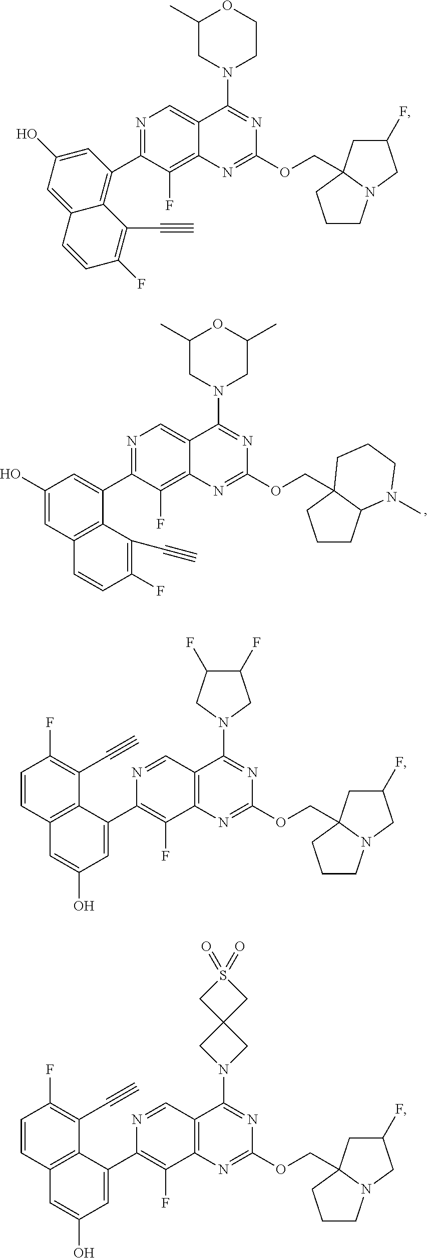 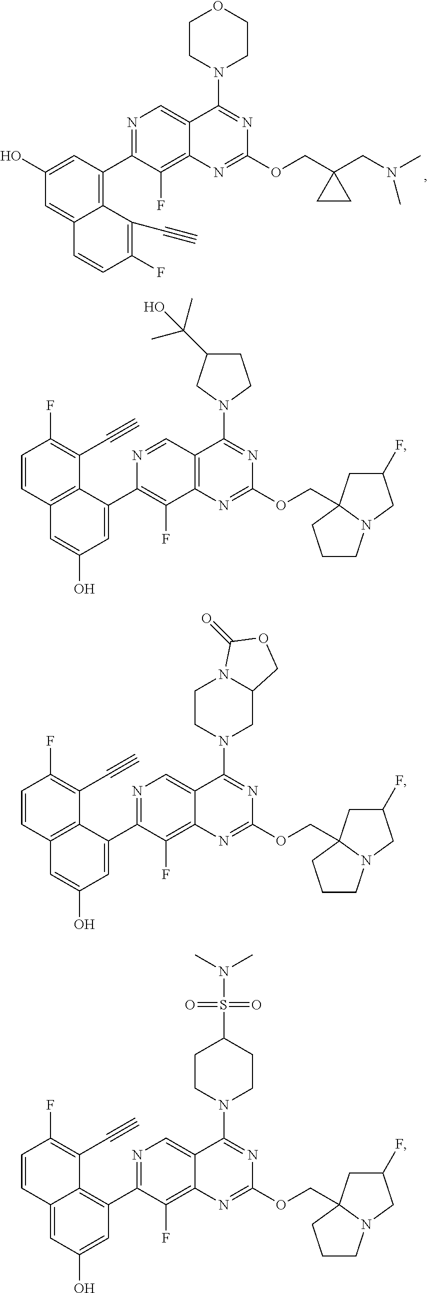 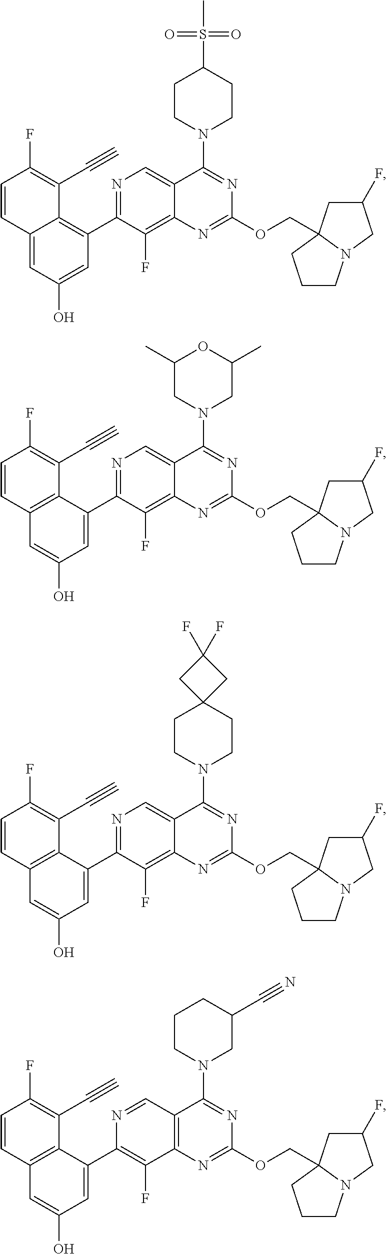 [SMILES: C#Cc1c(F)ccc2cc(O)cc(-c3ncc4c(N5CCC(S(C)(=O)=O)CC5)nc(OCC56CCCN5CC(F)C6)nc4c3F)c12 ; C#Cc1c(F)ccc2cc(O)cc(-c3ncc4c(N5CC(C)OC(C)C5)nc(OCC56CCCN5CC(F)C6)nc4c3F)c12 ; C#Cc1c(F)ccc2cc(O)cc(-c3ncc4c(N5CCC6(CC5)CC(F)(F)C6)nc(OCC56CCCN5CC(F)C6)nc4c3F)c12 ; C#Cc1c(F)ccc2cc(O)cc(-c3ncc4c(N5CCCC(C#N)C5)nc(OCC56CCCN5CC(F)C6)nc4c3F)c12] 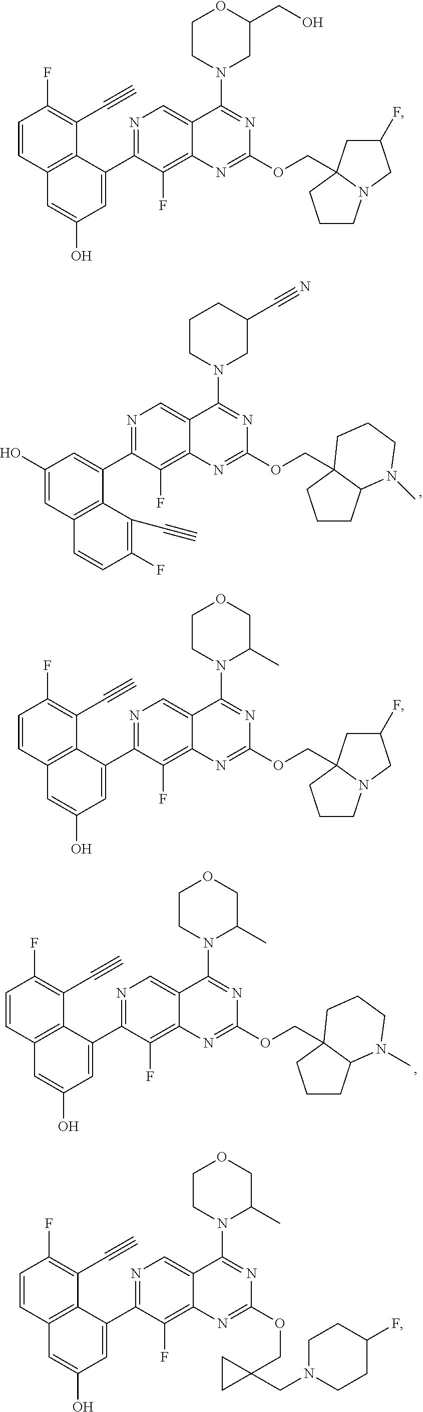 [SMILES: C#Cc1c(F)ccc2cc(O)cc(-c3ncc4c(N5CCOC(CO)C5)nc(OCC56CCCN5CC(F)C6)nc4c3F)c12 ; C#Cc1c(F)ccc2cc(O)cc(-c3ncc4c(N5CCCC(C#N)C5)nc(OCC56CCCC5N(C)CCC6)nc4c3F)c12 ; C#Cc1c(F)ccc2cc(O)cc(-c3ncc4c(N5CCOCC5C)nc(OCC56CCCN5CC(F)C6)nc4c3F)c12 ; C#Cc1c(F)ccc2cc(O)cc(-c3ncc4c(N5CCOCC5C)nc(OCC56CCCC5N(C)CCC6)nc4c3F)c12 ; C#Cc1c(F)ccc2cc(O)cc(-c3ncc4c(N5CCOCC5C)nc(OCC5(CN6CCC(F)CC6)CC5)nc4c3F)c12] 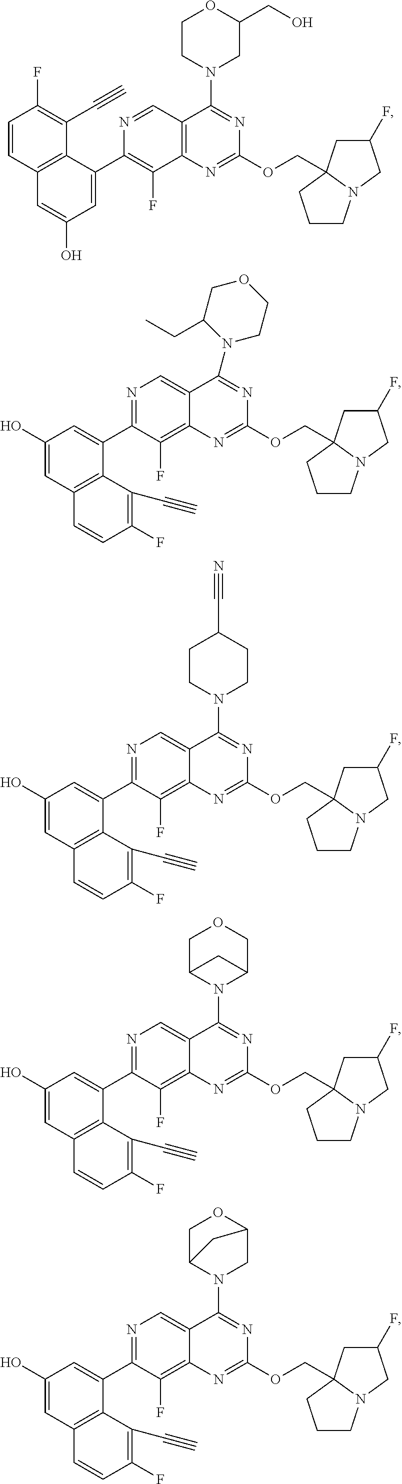 [SMILES: C#Cc1c(F)ccc2cc(O)cc(-c3ncc4c(N5CCOC(CO)C5)nc(OCC56CCCN5CC(F)C6)nc4c3F)c12 ; C#Cc1c(F)ccc2cc(O)cc(-c3ncc4c(N5CCOCC5CC)nc(OCC56CCCN5CC(F)C6)nc4c3F)c12 ; C#Cc1c(F)ccc2cc(O)cc(-c3ncc4c(N5CCC(C#N)CC5)nc(OCC56CCCN5CC(F)C6)nc4c3F)c12 ; C#Cc1c(F)ccc2cc(O)cc(-c3ncc4c(N5C6COCC5C6)nc(OCC56CCCN5CC(F)C6)nc4c3F)c12 ; C#Cc1c(F)ccc2cc(O)cc(-c3ncc4c(N5CC6CC5CO6)nc(OCC56CCCN5CC(F)C6)nc4c3F)c12] 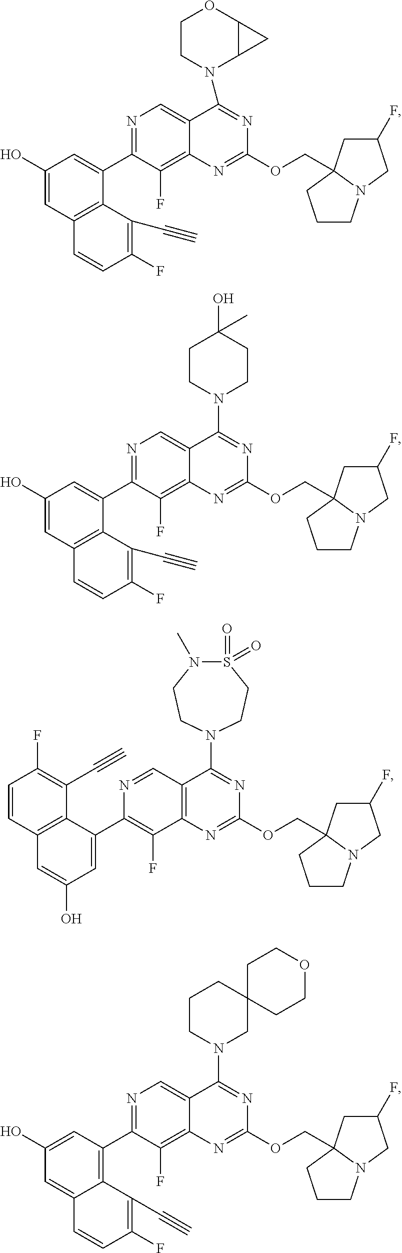 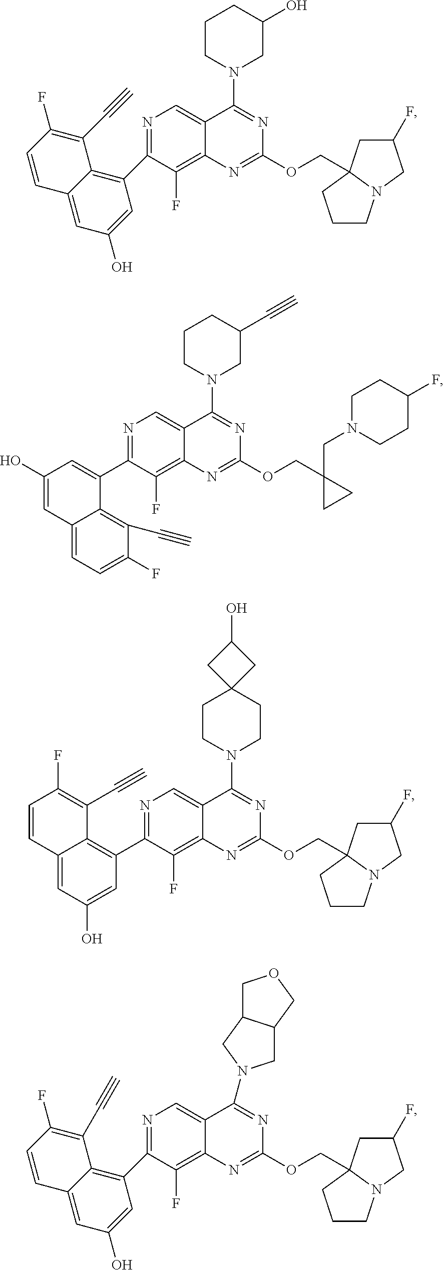 [SMILES: C#Cc1c(F)ccc2cc(O)cc(-c3ncc4c(N5CCCC(O)C5)nc(OCC56CCCN5CC(F)C6)nc4c3F)c12 ; C#Cc1c(F)ccc2cc(O)cc(-c3ncc4c(N5CCCC(C#C)C5)nc(OCC5(CN6CCC(F)CC6)CC5)nc4c3F)c12 ; C#Cc1c(F)ccc2cc(O)cc(-c3ncc4c(N5CCC6(CC5)CC(O)C6)nc(OCC56CCCN5CC(F)C6)nc4c3F)c12 ; C#Cc1c(F)ccc2cc(O)cc(-c3ncc4c(N5CC6COCC6C5)nc(OCC56CCCN5CC(F)C6)nc4c3F)c12] 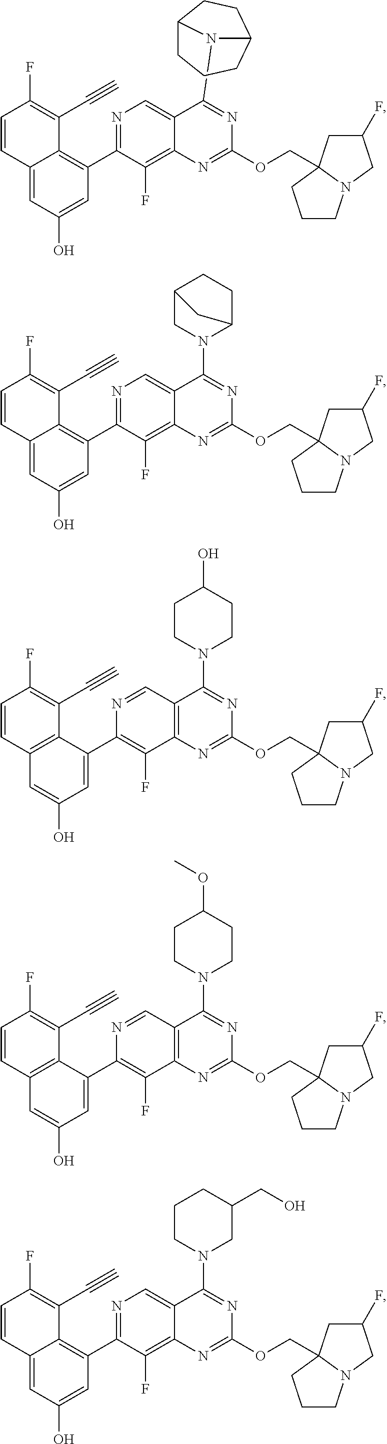 [SMILES: C#Cc1c(F)ccc2cc(O)cc(-c3ncc4c(N5C6CCCC5CC6)nc(OCC56CCCN5CC(F)C6)nc4c3F)c12 ; C#Cc1c(F)ccc2cc(O)cc(-c3ncc4c(N5CC6CCC5C6)nc(OCC56CCCN5CC(F)C6)nc4c3F)c12 ; C#Cc1c(F)ccc2cc(O)cc(-c3ncc4c(N5CCC(O)CC5)nc(OCC56CCCN5CC(F)C6)nc4c3F)c12 ; C#Cc1c(F)ccc2cc(O)cc(-c3ncc4c(N5CCC(OC)CC5)nc(OCC56CCCN5CC(F)C6)nc4c3F)c12 ; C#Cc1c(F)ccc2cc(O)cc(-c3ncc4c(N5CCCC(CO)C5)nc(OCC56CCCN5CC(F)C6)nc4c3F)c12] 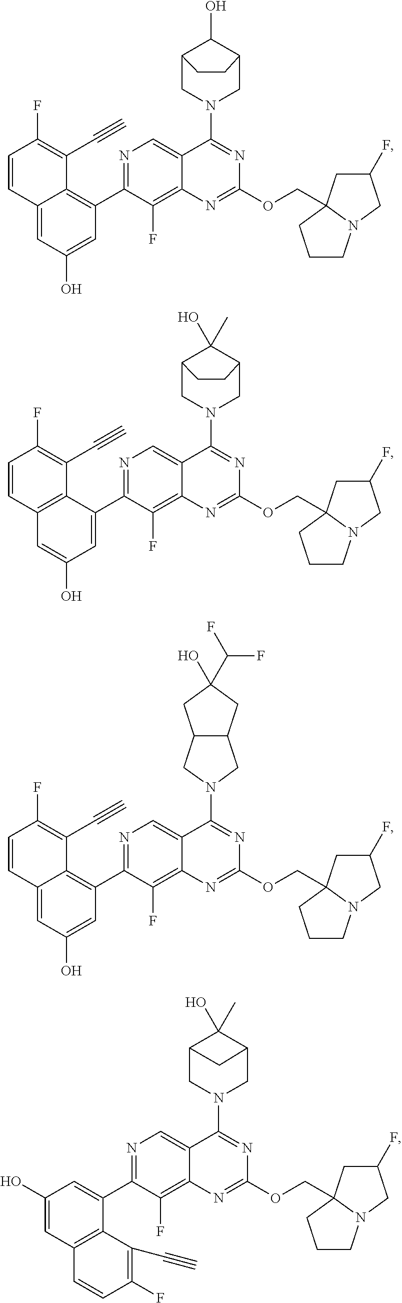 [SMILES: C#Cc1c(F)ccc2cc(O)cc(-c3ncc4c(N5CC6CCC(C5)C6O)nc(OCC56CCCN5CC(F)C6)nc4c3F)c12 ; C#Cc1c(F)ccc2cc(O)cc(-c3ncc4c(N5CC6CCC(C5)C6(C)O)nc(OCC56CCCN5CC(F)C6)nc4c3F)c12 ; C#Cc1c(F)ccc2cc(O)cc(-c3ncc4c(N5CC6CC(O)(C(F)F)CC6C5)nc(OCC56CCCN5CC(F)C6)nc4c3F)c12 ; C#Cc1c(F)ccc2cc(O)cc(-c3ncc4c(N5CC6CC(C5)C6(C)O)nc(OCC56CCCN5CC(F)C6)nc4c3F)c12] 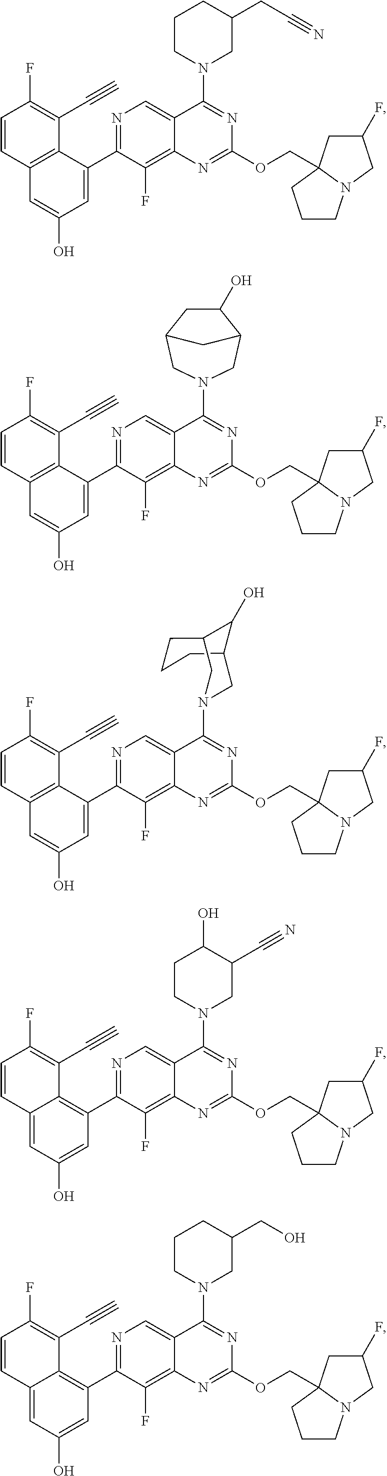 [SMILES: C#Cc1c(F)ccc2cc(O)cc(-c3ncc4c(N5CCCC(CC#N)C5)nc(OCC56CCCN5CC(F)C6)nc4c3F)c12 ; C#Cc1c(F)ccc2cc(O)cc(-c3ncc4c(N5CC6CC(O)C(C6)C5)nc(OCC56CCCN5CC(F)C6)nc4c3F)c12 ; C#Cc1c(F)ccc2cc(O)cc(-c3ncc4c(N5CC6CCCC(C5)C6O)nc(OCC56CCCN5CC(F)C6)nc4c3F)c12 ; C#Cc1c(F)ccc2cc(O)cc(-c3ncc4c(N5CCC(O)C(C#N)C5)nc(OCC56CCCN5CC(F)C6)nc4c3F)c12 ; C#Cc1c(F)ccc2cc(O)cc(-c3ncc4c(N5CCCC(CO)C5)nc(OCC56CCCN5CC(F)C6)nc4c3F)c12] 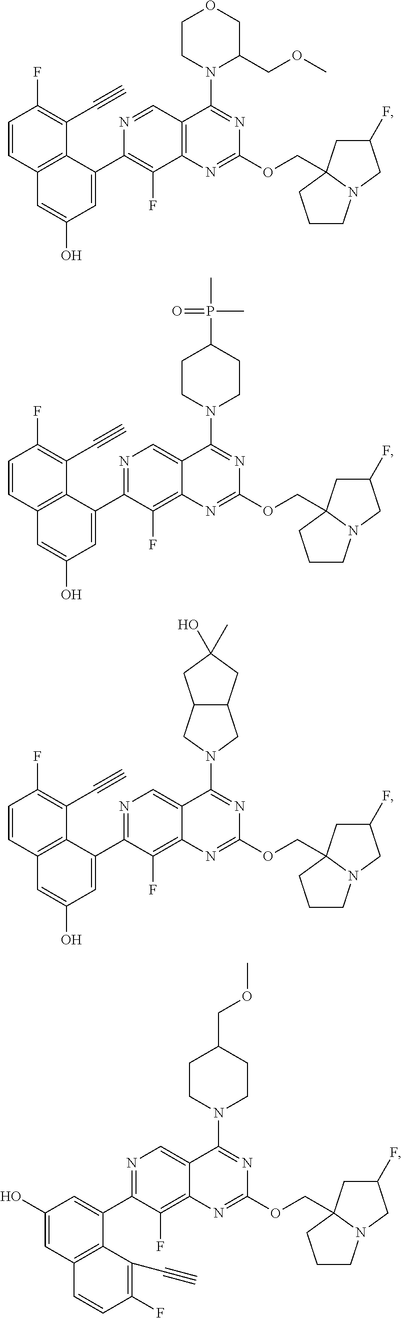 [SMILES: C#Cc1c(F)ccc2cc(O)cc(-c3ncc4c(N5CCOCC5COC)nc(OCC56CCCN5CC(F)C6)nc4c3F)c12 ; C#Cc1c(F)ccc2cc(O)cc(-c3ncc4c(N5CCC(P(C)(C)=O)CC5)nc(OCC56CCCN5CC(F)C6)nc4c3F)c12 ; C#Cc1c(F)ccc2cc(O)cc(-c3ncc4c(N5CC6CC(C)(O)CC6C5)nc(OCC56CCCN5CC(F)C6)nc4c3F)c12 ; C#Cc1c(F)ccc2cc(O)cc(-c3ncc4c(N5CCC(COC)CC5)nc(OCC56CCCN5CC(F)C6)nc4c3F)c12] 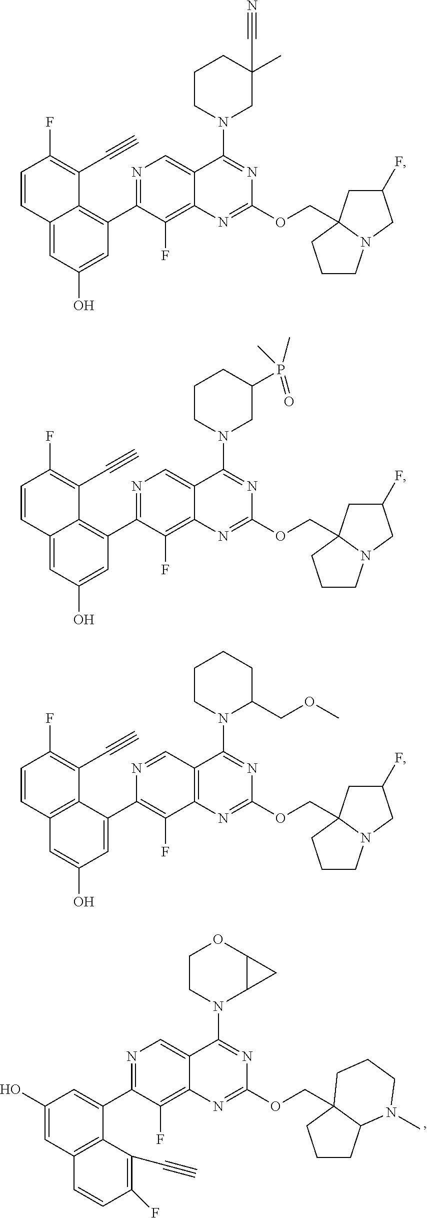 [SMILES: C#Cc1c(F)ccc2cc(O)cc(-c3ncc4c(N5CCCC(C)(C#N)C5)nc(OCC56CCCN5CC(F)C6)nc4c3F)c12 ; C#Cc1c(F)ccc2cc(O)cc(-c3ncc4c(N5CCCC(P(C)(C)=O)C5)nc(OCC56CCCN5CC(F)C6)nc4c3F)c12 ; C#Cc1c(F)ccc2cc(O)cc(-c3ncc4c(N5CCCCC5COC)nc(OCC56CCCN5CC(F)C6)nc4c3F)c12 ; C#Cc1c(F)ccc2cc(O)cc(-c3ncc4c(N5CCOC6CC65)nc(OCC56CCCC5N(C)CCC6)nc4c3F)c12] 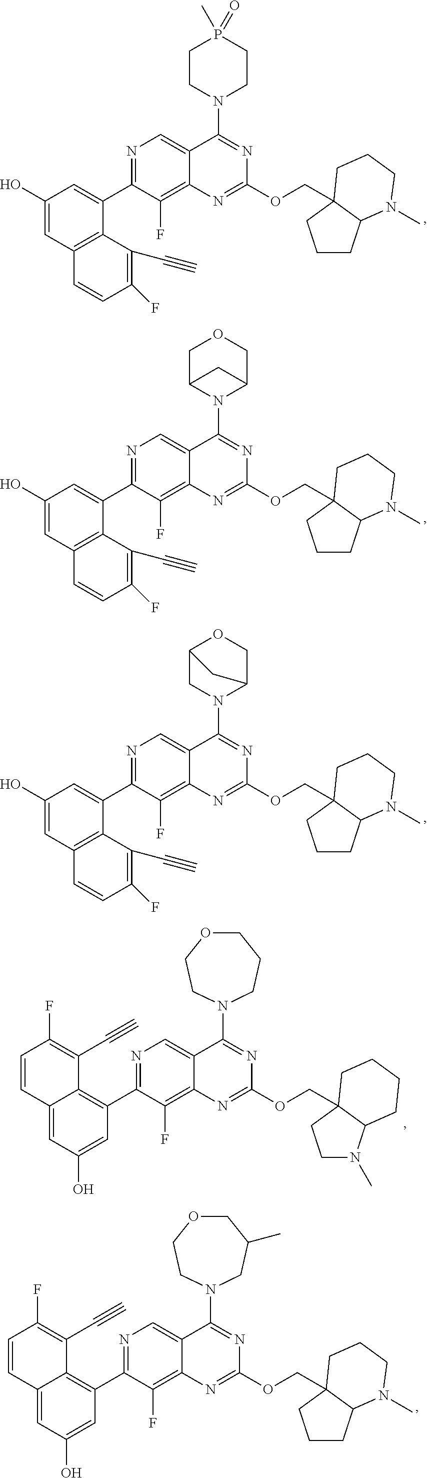 [SMILES: C#Cc1c(F)ccc2cc(O)cc(-c3ncc4c(N5CCP(C)(=O)CC5)nc(OCC56CCCC5N(C)CCC6)nc4c3F)c12 ; C#Cc1c(F)ccc2cc(O)cc(-c3ncc4c(N5C6COCC5C6)nc(OCC56CCCC5N(C)CCC6)nc4c3F)c12 ; C#Cc1c(F)ccc2cc(O)cc(-c3ncc4c(N5CC6CC5CO6)nc(OCC56CCCC5N(C)CCC6)nc4c3F)c12 ; C#Cc1c(F)ccc2cc(O)cc(-c3ncc4c(N5CCCOCC5)nc(OCC56CCCCC5N(C)CC6)nc4c3F)c12 ; C#Cc1c(F)ccc2cc(O)cc(-c3ncc4c(N5CCOCC(C)C5)nc(OCC56CCCC5N(C)CCC6)nc4c3F)c12] 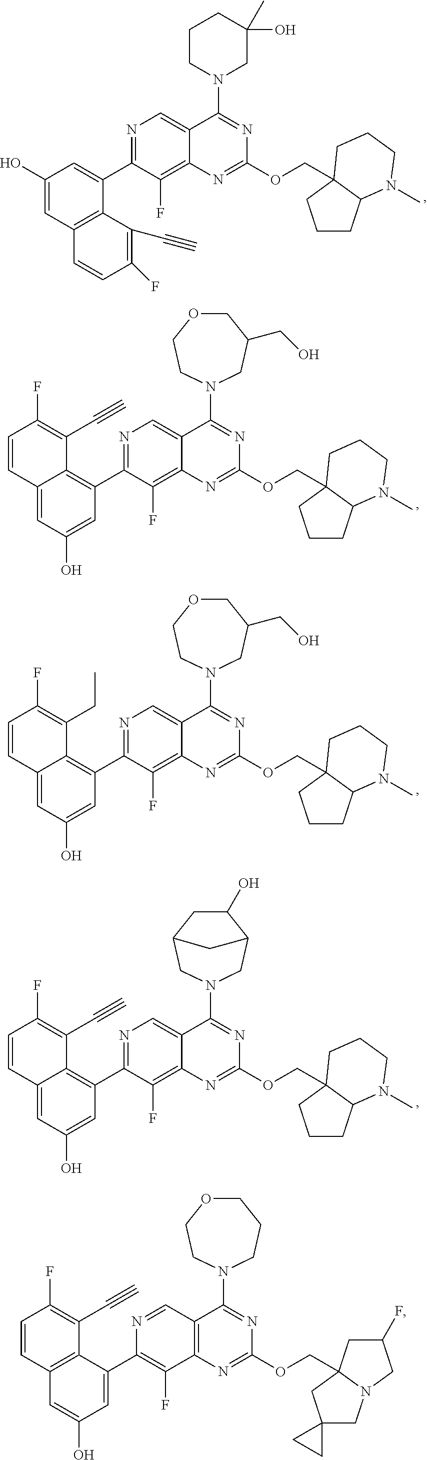 [SMILES: C#Cc1c(F)ccc2cc(O)cc(-c3ncc4c(N5CCCC(C)(O)C5)nc(OCC56CCCC5N(C)CCC6)nc4c3F)c12 ; C#Cc1c(F)ccc2cc(O)cc(-c3ncc4c(N5CCOCC(CO)C5)nc(OCC56CCCC5N(C)CCC6)nc4c3F)c12 ; CCc1c(F)ccc2cc(O)cc(-c3ncc4c(N5CCOCC(CO)C5)nc(OCC56CCCC5N(C)CCC6)nc4c3F)c12 ; C#Cc1c(F)ccc2cc(O)cc(-c3ncc4c(N5CC6CC(O)C(C6)C5)nc(OCC56CCCC5N(C)CCC6)nc4c3F)c12 ; C#Cc1c(F)ccc2cc(O)cc(-c3ncc4c(N5CCCOCC5)nc(OCC56CC(F)CN5CC5(CC5)C6)nc4c3F)c12] 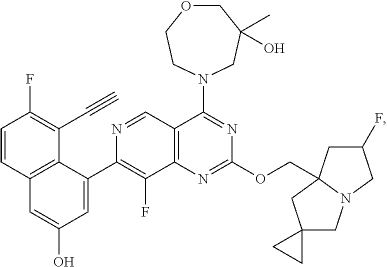 [SMILES: C#Cc1c(F)ccc2cc(O)cc(-c3ncc4c(N5CCOCC(C)(O)C5)nc(OCC56CC(F)CN5CC5(CC5)C6)nc4c3F)c12] 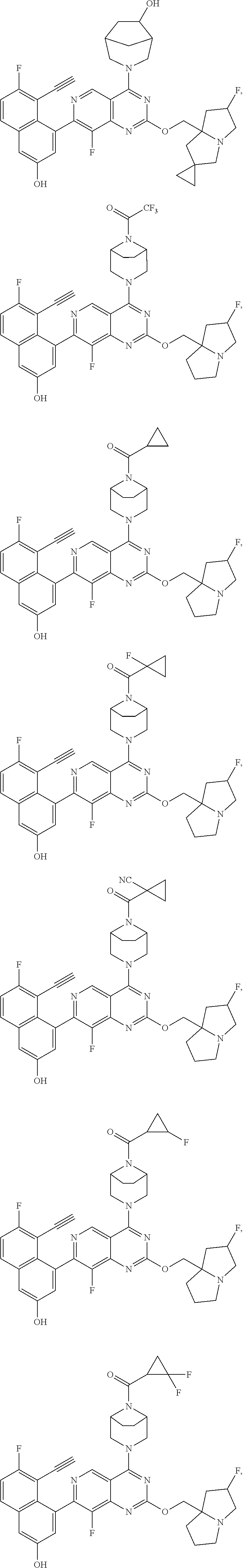 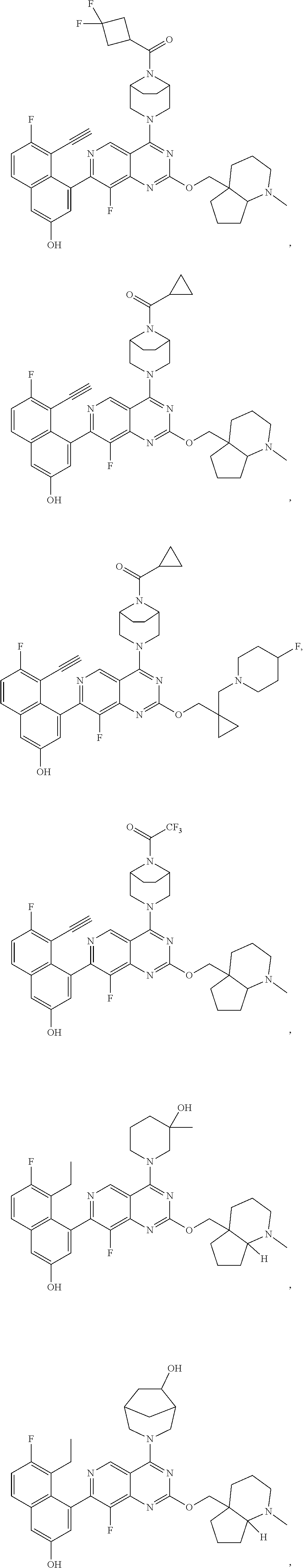 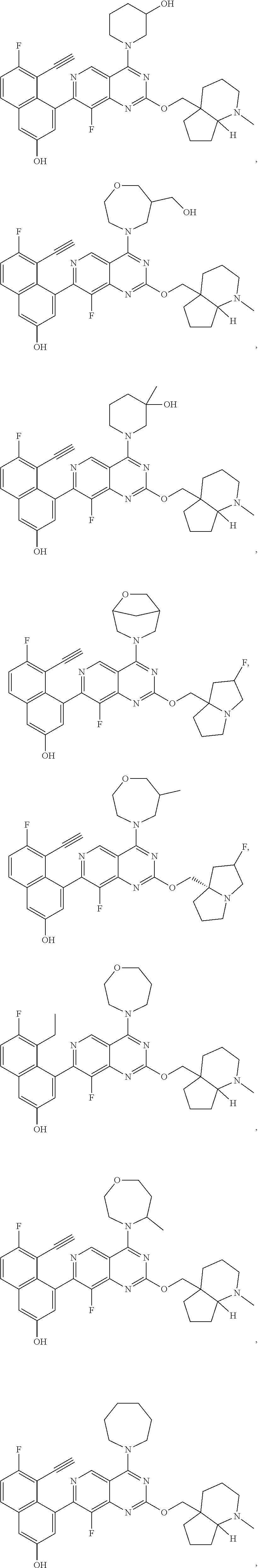 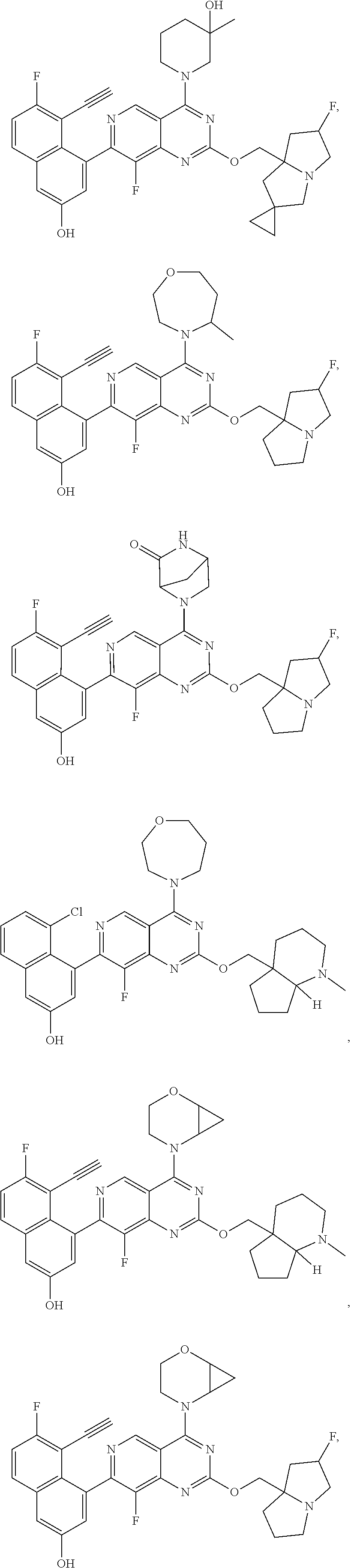 [SMILES: C#Cc1c(F)ccc2cc(O)cc(-c3ncc4c(N5CCCC(C)(O)C5)nc(OCC56CC(F)CN5CC5(CC5)C6)nc4c3F)c12 ; C#Cc1c(F)ccc2cc(O)cc(-c3ncc4c(N5CCOCCC5C)nc(OCC56CCCN5CC(F)C6)nc4c3F)c12 ; C#Cc1c(F)ccc2cc(O)cc(-c3ncc4c(N5CC6CC5C(=O)N6)nc(OCC56CCCN5CC(F)C6)nc4c3F)c12 ; CN1CCCC2(COc3nc(N4CCCOCC4)c4cnc(-c5cc(O)cc6cccc(Cl)c56)c(F)c4n3)CCCC12 ; C#Cc1c(F)ccc2cc(O)cc(-c3ncc4c(N5CCOC6CC65)nc(OCC56CCCC5N(C)CCC6)nc4c3F)c12 ; C#Cc1c(F)ccc2cc(O)cc(-c3ncc4c(N5CCOC6CC65)nc(OCC56CCCN5CC(F)C6)nc4c3F)c12] 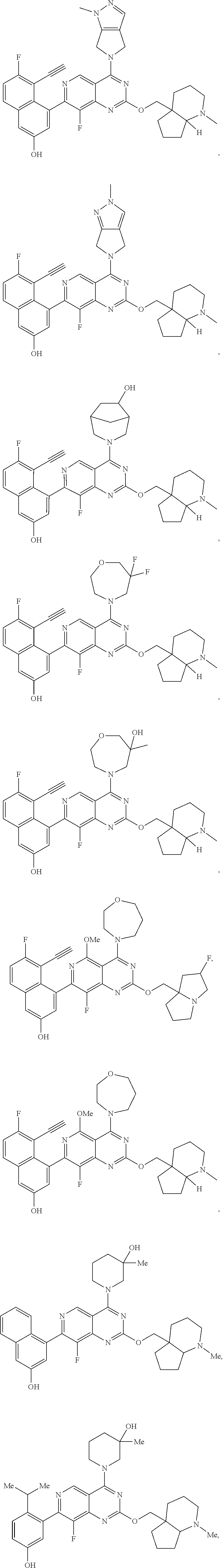 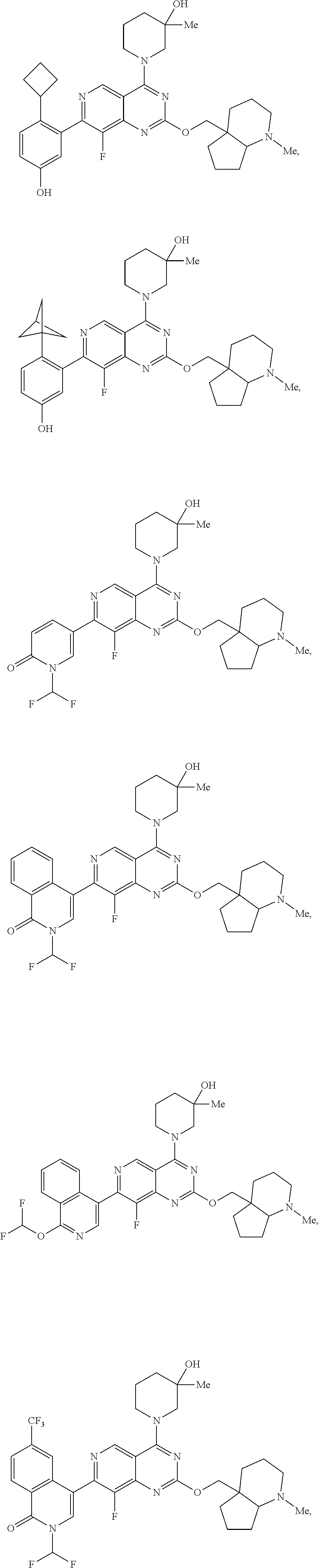 [SMILES: CN1CCCC2(COc3nc(N4CCCC(C)(O)C4)c4cnc(-c5cc(O)ccc5C5CCC5)c(F)c4n3)CCCC12 ; CN1CCCC2(COc3nc(N4CCCC(C)(O)C4)c4cnc(-c5cc(O)ccc5C56CC(C5)C6)c(F)c4n3)CCCC12 ; CN1CCCC2(COc3nc(N4CCCC(C)(O)C4)c4cnc(-c5ccc(=O)n(C(F)F)c5)c(F)c4n3)CCCC12 ; CN1CCCC2(COc3nc(N4CCCC(C)(O)C4)c4cnc(-c5cn(C(F)F)c(=O)c6ccccc56)c(F)c4n3)CCCC12 ; CN1CCCC2(COc3nc(N4CCCC(C)(O)C4)c4cnc(-c5cnc(OC(F)F)c6ccccc56)c(F)c4n3)CCCC12 ; CN1CCCC2(COc3nc(N4CCCC(C)(O)C4)c4cnc(-c5cn(C(F)F)c(=O)c6ccc(C(F)(F)F)cc56)c(F)c4n3)CCCC12] 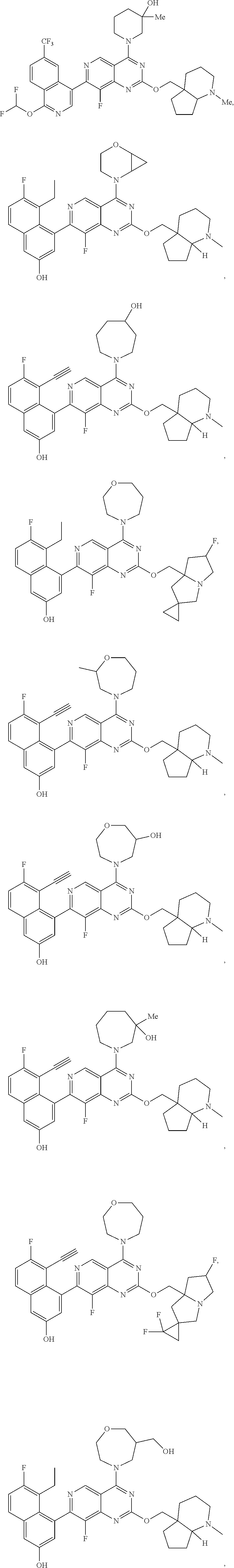 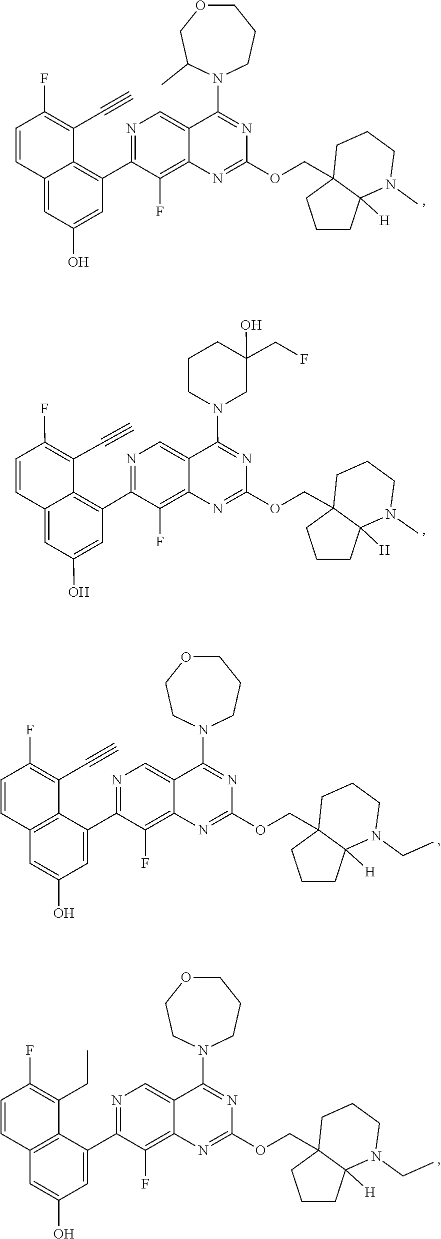 [SMILES: C#Cc1c(F)ccc2cc(O)cc(-c3ncc4c(N5CCCOCC5C)nc(OCC56CCCC5N(C)CCC6)nc4c3F)c12 ; C#Cc1c(F)ccc2cc(O)cc(-c3ncc4c(N5CCCC(O)(CF)C5)nc(OCC56CCCC5N(C)CCC6)nc4c3F)c12 ; C#Cc1c(F)ccc2cc(O)cc(-c3ncc4c(N5CCCOCC5)nc(OCC56CCCC5N(CC)CCC6)nc4c3F)c12 ; CCc1c(F)ccc2cc(O)cc(-c3ncc4c(N5CCCOCC5)nc(OCC56CCCC5N(CC)CCC6)nc4c3F)c12] 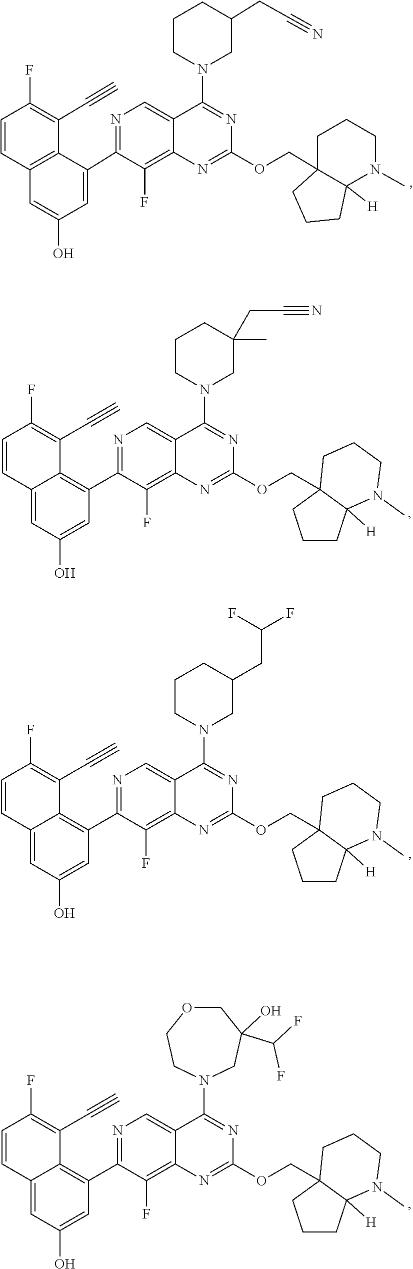 [SMILES: C#Cc1c(F)ccc2cc(O)cc(-c3ncc4c(N5CCCC(CC#N)C5)nc(OCC56CCCC5N(C)CCC6)nc4c3F)c12 ; C#Cc1c(F)ccc2cc(O)cc(-c3ncc4c(N5CCCC(C)(CC#N)C5)nc(OCC56CCCC5N(C)CCC6)nc4c3F)c12 ; C#Cc1c(F)ccc2cc(O)cc(-c3ncc4c(N5CCCC(CC(F)F)C5)nc(OCC56CCCC5N(C)CCC6)nc4c3F)c12 ; C#Cc1c(F)ccc2cc(O)cc(-c3ncc4c(N5CCOCC(O)(C(F)F)C5)nc(OCC56CCCC5N(C)CCC6)nc4c3F)c12] 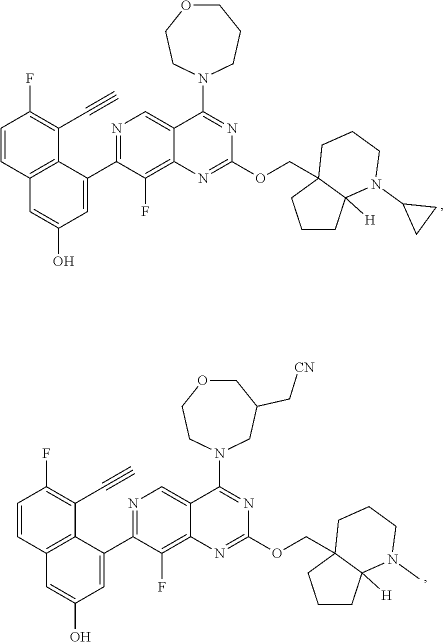 [SMILES: C#Cc1c(F)ccc2cc(O)cc(-c3ncc4c(N5CCCOCC5)nc(OCC56CCCC5N(C5CC5)CCC6)nc4c3F)c12 ; C#Cc1c(F)ccc2cc(O)cc(-c3ncc4c(N5CCOCC(CC#N)C5)nc(OCC56CCCC5N(C)CCC6)nc4c3F)c12] 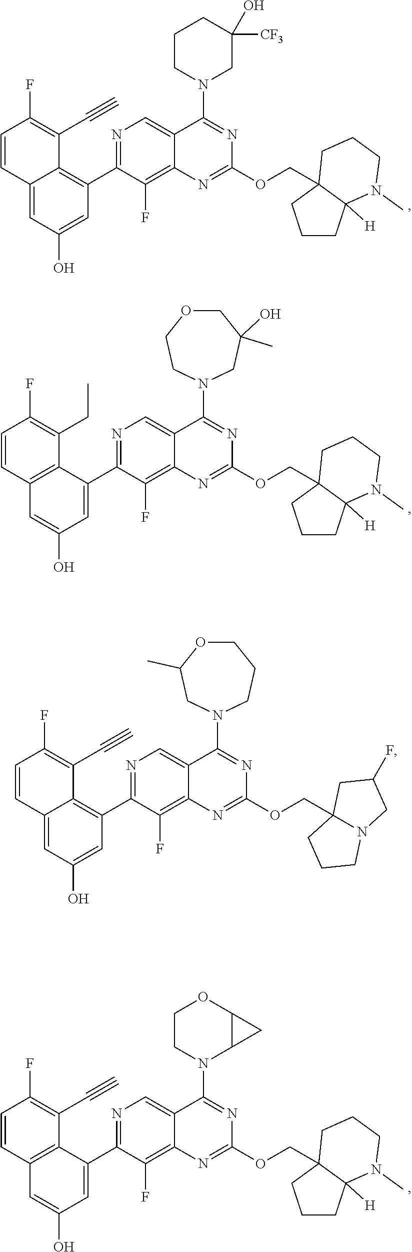 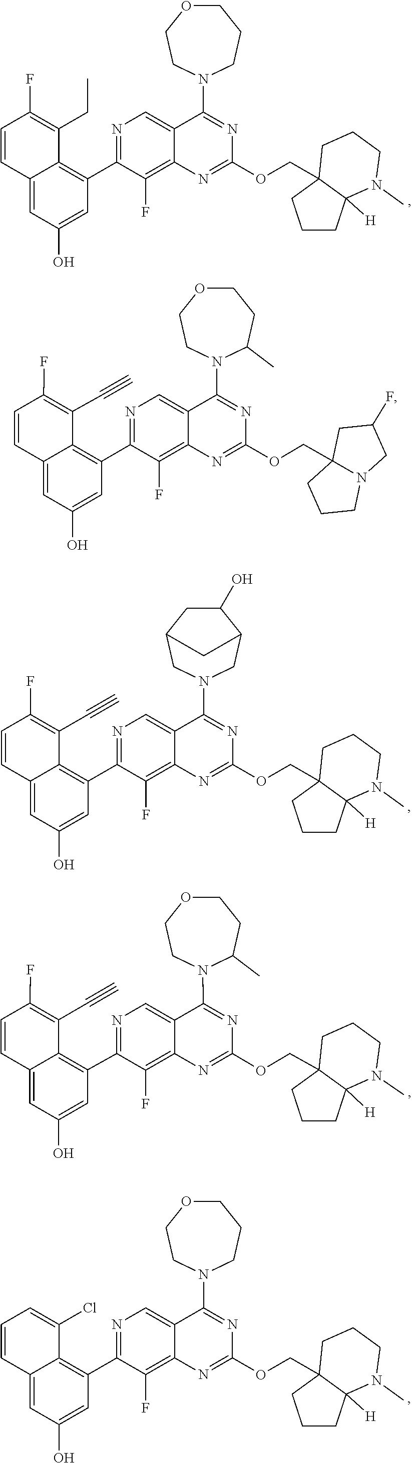 [SMILES: CCc1c(F)ccc2cc(O)cc(-c3ncc4c(N5CCCOCC5)nc(OCC56CCCC5N(C)CCC6)nc4c3F)c12 ; C#Cc1c(F)ccc2cc(O)cc(-c3ncc4c(N5CCOCCC5C)nc(OCC56CCCN5CC(F)C6)nc4c3F)c12 ; C#Cc1c(F)ccc2cc(O)cc(-c3ncc4c(N5CC6CC(O)C(C6)C5)nc(OCC56CCCC5N(C)CCC6)nc4c3F)c12 ; C#Cc1c(F)ccc2cc(O)cc(-c3ncc4c(N5CCOCCC5C)nc(OCC56CCCC5N(C)CCC6)nc4c3F)c12 ; CN1CCCC2(COc3nc(N4CCCOCC4)c4cnc(-c5cc(O)cc6cccc(Cl)c56)c(F)c4n3)CCCC12] 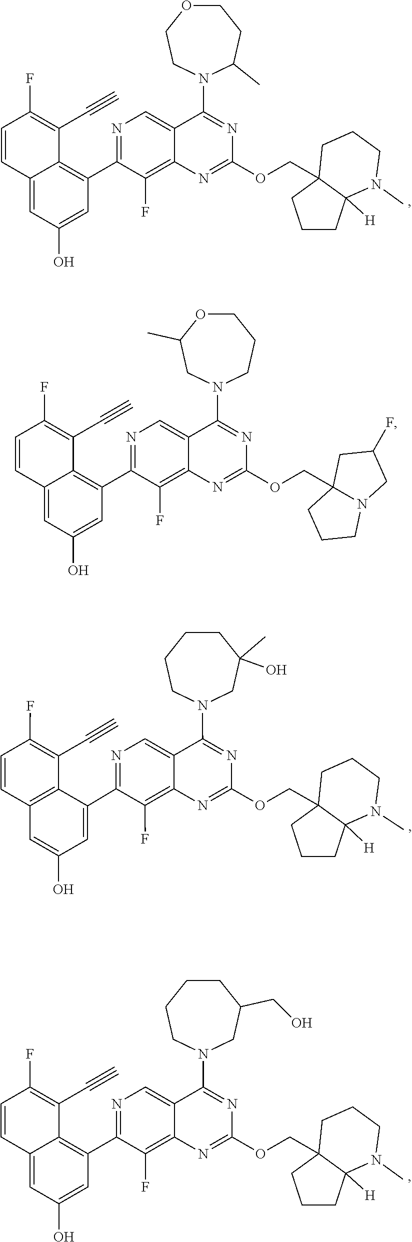 [SMILES: C#Cc1c(F)ccc2cc(O)cc(-c3ncc4c(N5CCOCCC5C)nc(OCC56CCCC5N(C)CCC6)nc4c3F)c12 ; C#Cc1c(F)ccc2cc(O)cc(-c3ncc4c(N5CCCOC(C)C5)nc(OCC56CCCN5CC(F)C6)nc4c3F)c12 ; C#Cc1c(F)ccc2cc(O)cc(-c3ncc4c(N5CCCCC(C)(O)C5)nc(OCC56CCCC5N(C)CCC6)nc4c3F)c12 ; C#Cc1c(F)ccc2cc(O)cc(-c3ncc4c(N5CCCCC(CO)C5)nc(OCC56CCCC5N(C)CCC6)nc4c3F)c12] 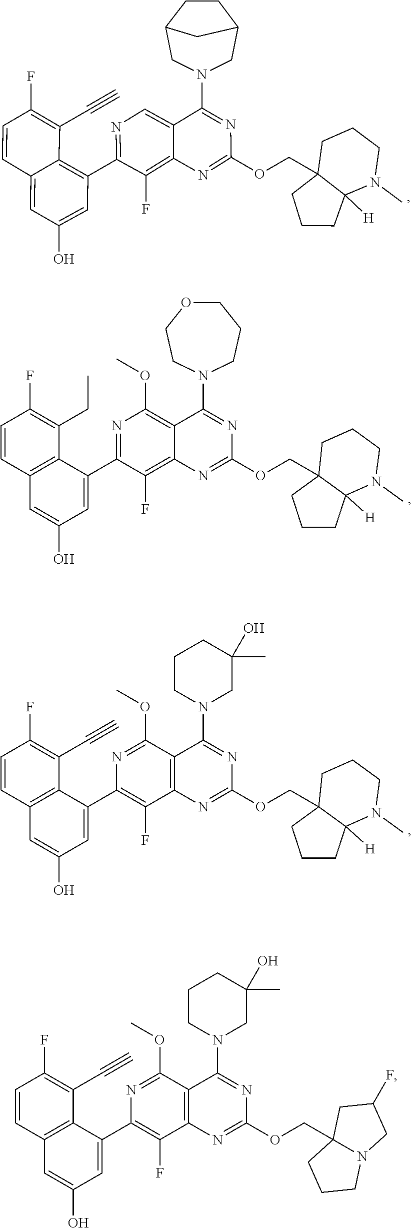 [SMILES: C#Cc1c(F)ccc2cc(O)cc(-c3ncc4c(N5CC6CCC(C6)C5)nc(OCC56CCCC5N(C)CCC6)nc4c3F)c12 ; CCc1c(F)ccc2cc(O)cc(-c3nc(OC)c4c(N5CCCOCC5)nc(OCC56CCCC5N(C)CCC6)nc4c3F)c12 ; C#Cc1c(F)ccc2cc(O)cc(-c3nc(OC)c4c(N5CCCC(C)(O)C5)nc(OCC56CCCC5N(C)CCC6)nc4c3F)c12 ; C#Cc1c(F)ccc2cc(O)cc(-c3nc(OC)c4c(N5CCCC(C)(O)C5)nc(OCC56CCCN5CC(F)C6)nc4c3F)c12] 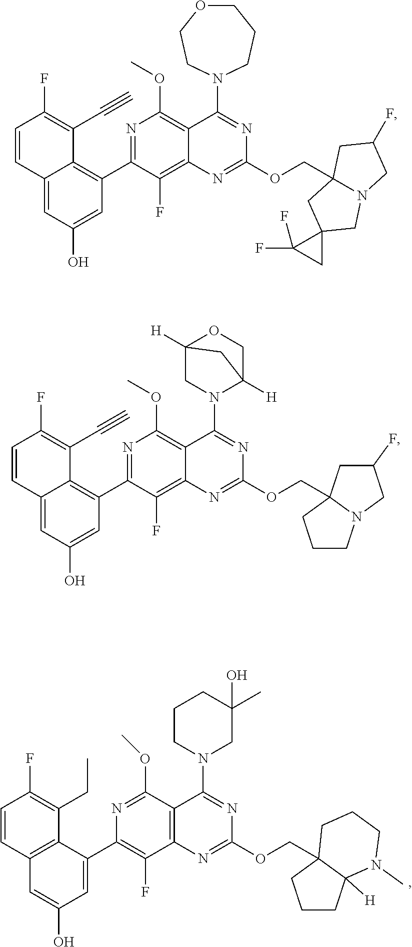 [SMILES: C#Cc1c(F)ccc2cc(O)cc(-c3nc(OC)c4c(N5CCCOCC5)nc(OCC56CC(F)CN5CC5(C6)CC5(F)F)nc4c3F)c12 ; C#Cc1c(F)ccc2cc(O)cc(-c3nc(OC)c4c(N5CC6CC5CO6)nc(OCC56CCCN5CC(F)C6)nc4c3F)c12 ; CCc1c(F)ccc2cc(O)cc(-c3nc(OC)c4c(N5CCCC(C)(O)C5)nc(OCC56CCCC5N(C)CCC6)nc4c3F)c12] 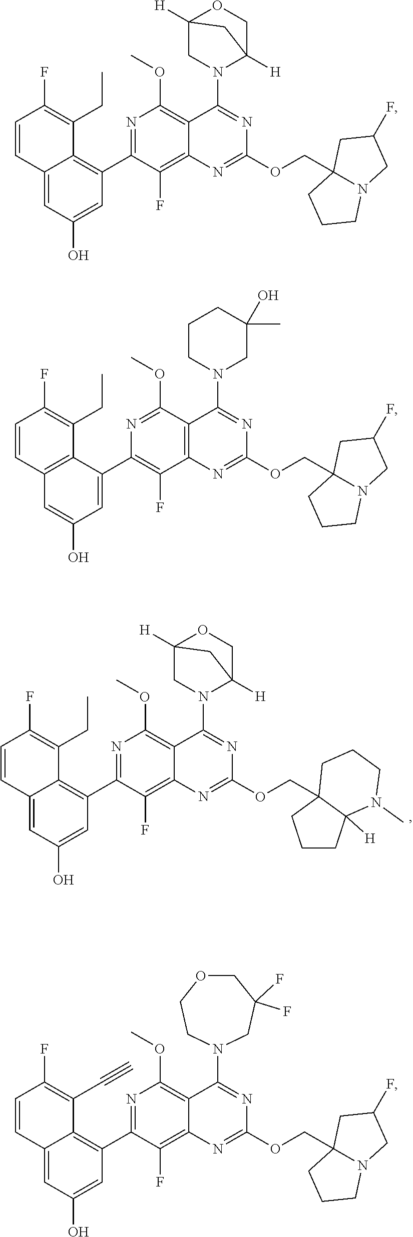 [SMILES: CCc1c(F)ccc2cc(O)cc(-c3nc(OC)c4c(N5CC6CC5CO6)nc(OCC56CCCN5CC(F)C6)nc4c3F)c12 ; CCc1c(F)ccc2cc(O)cc(-c3nc(OC)c4c(N5CCCC(C)(O)C5)nc(OCC56CCCN5CC(F)C6)nc4c3F)c12 ; CCc1c(F)ccc2cc(O)cc(-c3nc(OC)c4c(N5CC6CC5CO6)nc(OCC56CCCC5N(C)CCC6)nc4c3F)c12 ; C#Cc1c(F)ccc2cc(O)cc(-c3nc(OC)c4c(N5CCOCC(F)(F)C5)nc(OCC56CCCN5CC(F)C6)nc4c3F)c12] 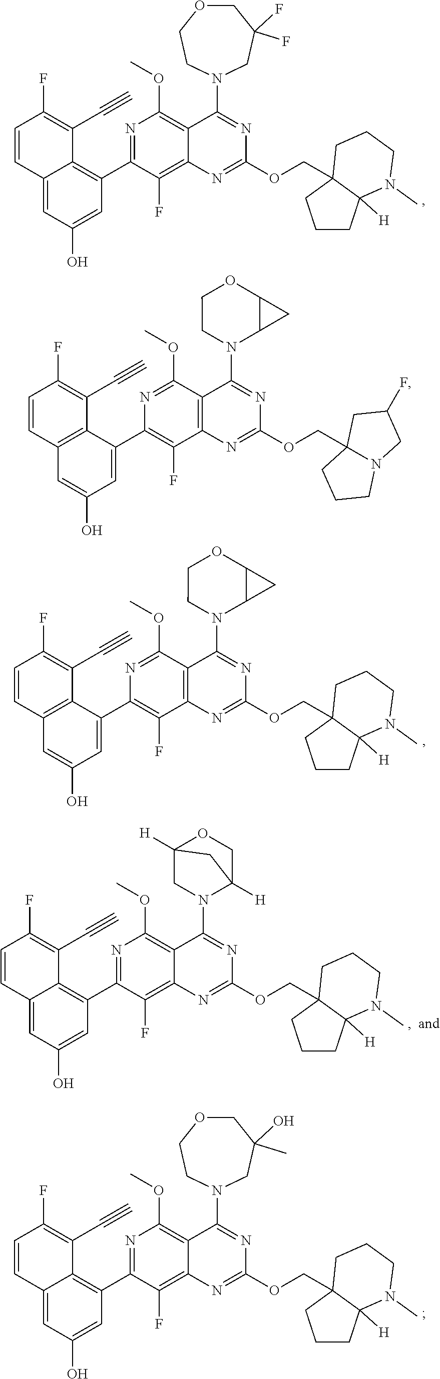
or a pharmaceutically acceptable salt thereof.

43. The compound of claim 42 that is selected from the group consisting of: 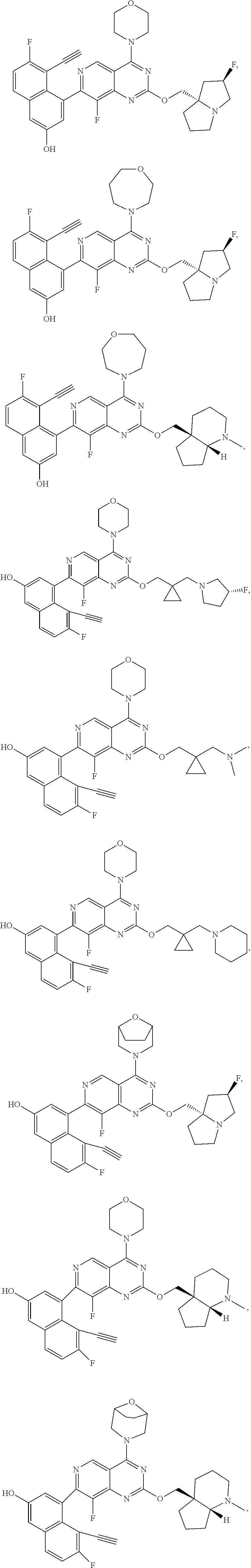 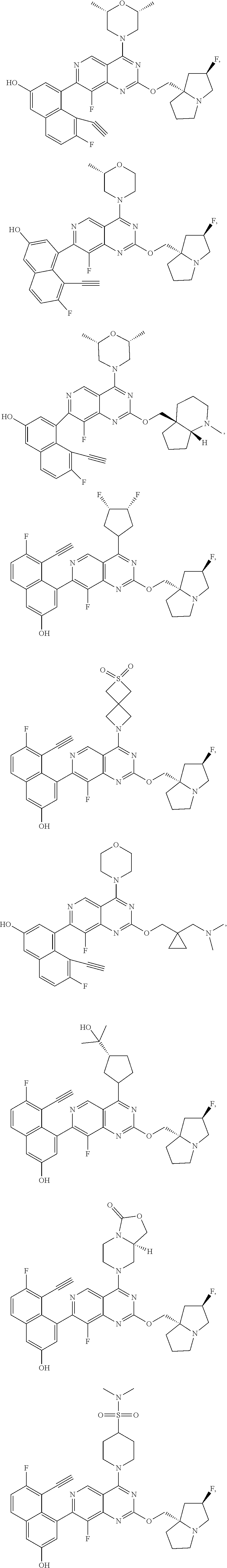 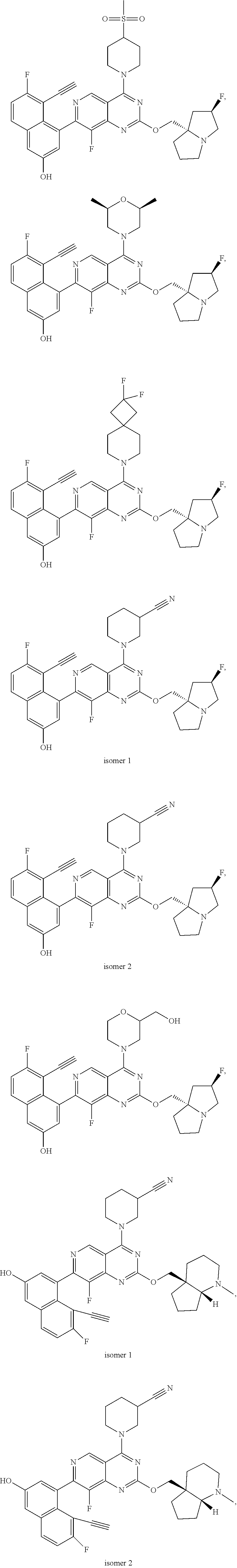 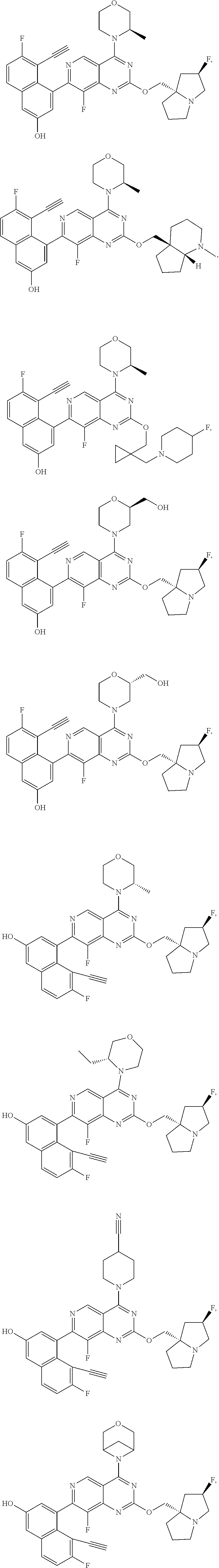 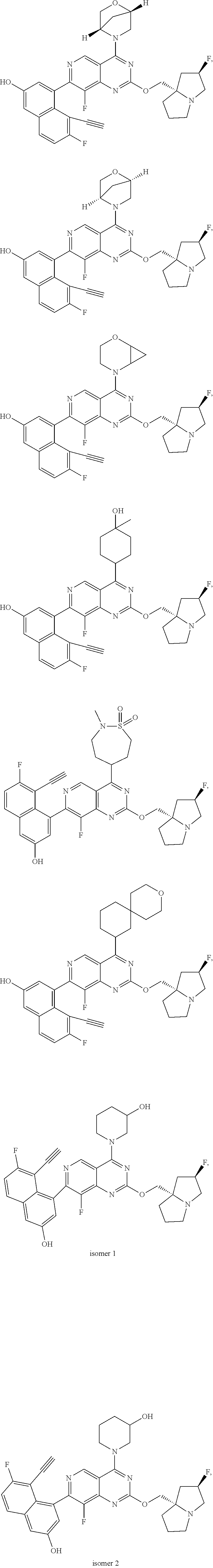 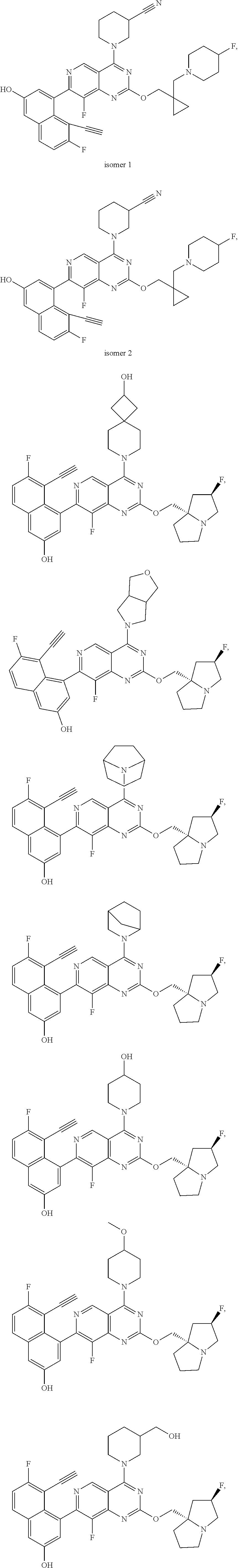 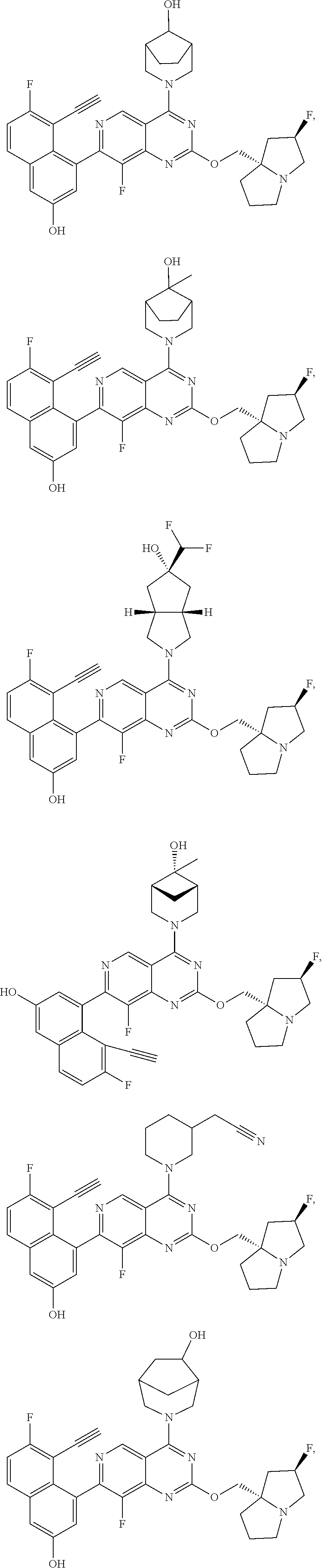 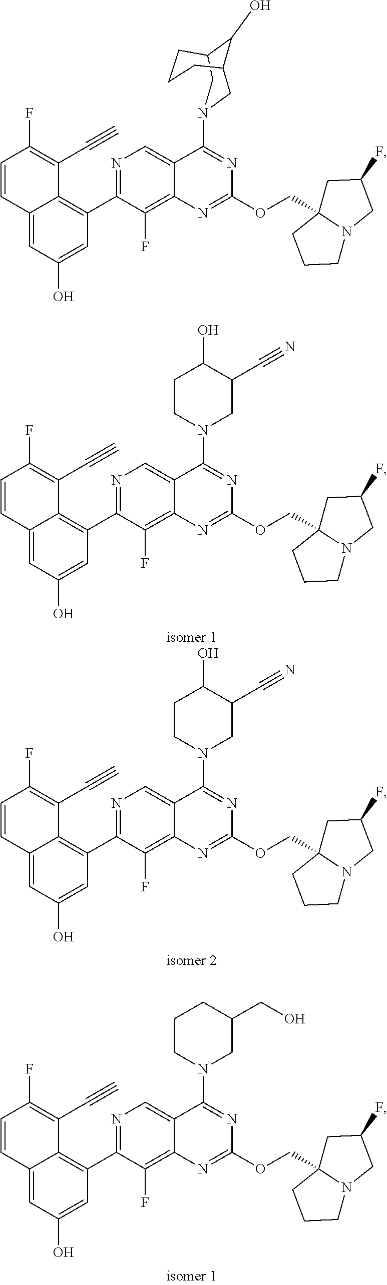 [SMILES: C#Cc1c(F)ccc2cc(O)cc(-c3ncc4c(N5CC6CCCC(C5)C6O)nc(OC[C@@]56CCCN5C[C@H](F)C6)nc4c3F)c12 ; C#Cc1c(F)ccc2cc(O)cc(-c3ncc4c(N5CCC(O)C(C#N)C5)nc(OC[C@@]56CCCN5C[C@H](F)C6)nc4c3F)c12 ; C#Cc1c(F)ccc2cc(O)cc(-c3ncc4c(N5CCC(O)C(C#N)C5)nc(OC[C@@]56CCCN5C[C@H](F)C6)nc4c3F)c12 ; C#Cc1c(F)ccc2cc(O)cc(-c3ncc4c(N5CCCC(CO)C5)nc(OC[C@@]56CCCN5C[C@H](F)C6)nc4c3F)c12] 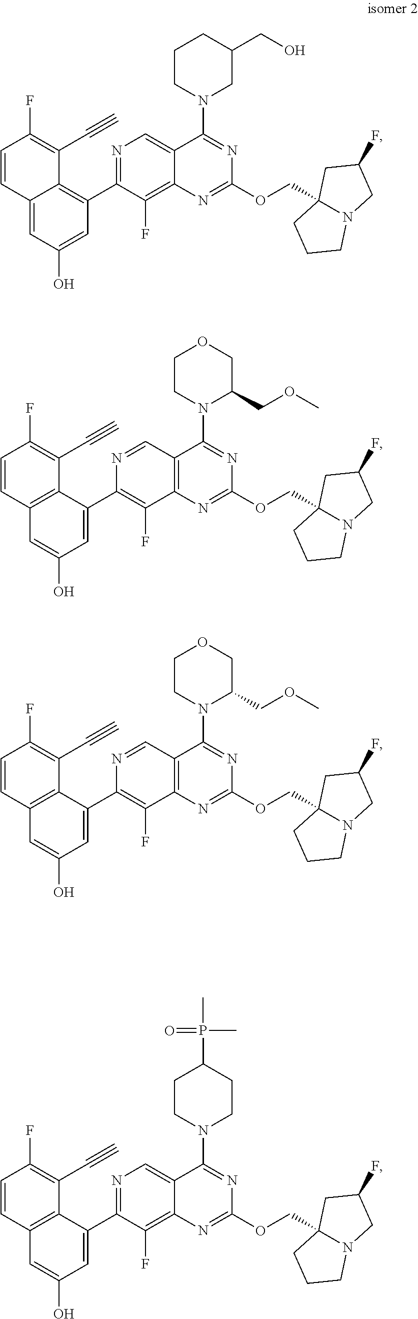 [SMILES: C#Cc1c(F)ccc2cc(O)cc(-c3ncc4c(N5CCCC(CO)C5)nc(OC[C@@]56CCCN5C[C@H](F)C6)nc4c3F)c12 ; C#Cc1c(F)ccc2cc(O)cc(-c3ncc4c(N5CCOC[C@H]5COC)nc(OC[C@@]56CCCN5C[C@H](F)C6)nc4c3F)c12 ; C#Cc1c(F)ccc2cc(O)cc(-c3ncc4c(N5CCOC[C@@H]5COC)nc(OC[C@@]56CCCN5C[C@H](F)C6)nc4c3F)c12 ; C#Cc1c(F)ccc2cc(O)cc(-c3ncc4c(N5CCC(P(C)(C)=O)CC5)nc(OC[C@@]56CCCN5C[C@H](F)C6)nc4c3F)c12] 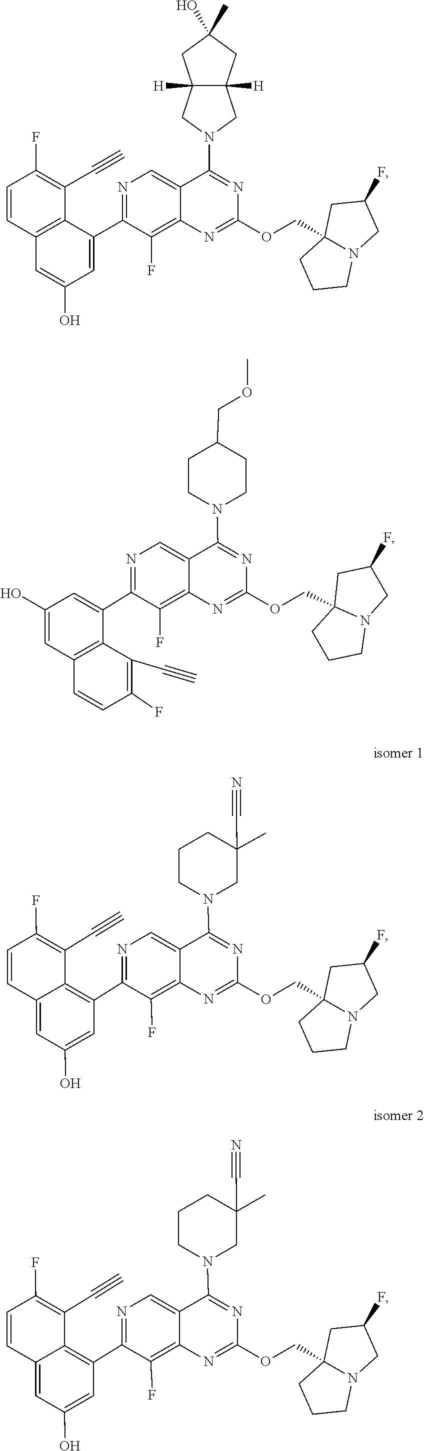 [SMILES: C#Cc1c(F)ccc2cc(O)cc(-c3ncc4c(N5C[C@@H]6C[C@](C)(O)C[C@@H]6C5)nc(OC[C@@]56CCCN5C[C@H](F)C6)nc4c3F)c12 ; C#Cc1c(F)ccc2cc(O)cc(-c3ncc4c(N5CCC(COC)CC5)nc(OC[C@@]56CCCN5C[C@H](F)C6)nc4c3F)c12 ; C#Cc1c(F)ccc2cc(O)cc(-c3ncc4c(N5CCCC(C)(C#N)C5)nc(OC[C@@]56CCCN5C[C@H](F)C6)nc4c3F)c12 ; C#Cc1c(F)ccc2cc(O)cc(-c3ncc4c(N5CCCC(C)(C#N)C5)nc(OC[C@@]56CCCN5C[C@H](F)C6)nc4c3F)c12] 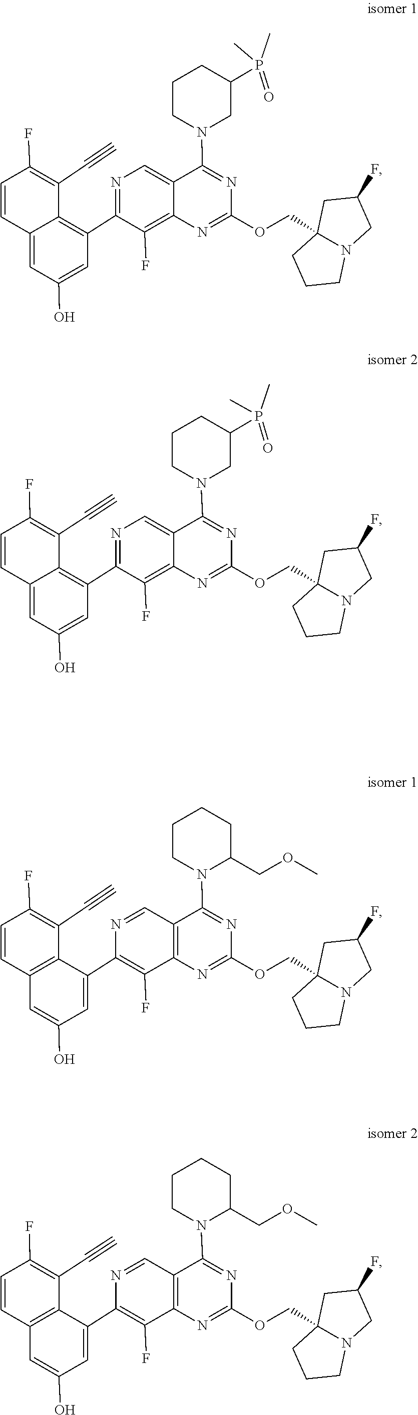 [SMILES: C#Cc1c(F)ccc2cc(O)cc(-c3ncc4c(N5CCCC(P(C)(C)=O)C5)nc(OC[C@@]56CCCN5C[C@H](F)C6)nc4c3F)c12 ; C#Cc1c(F)ccc2cc(O)cc(-c3ncc4c(N5CCCC(P(C)(C)=O)C5)nc(OC[C@@]56CCCN5C[C@H](F)C6)nc4c3F)c12 ; C#Cc1c(F)ccc2cc(O)cc(-c3ncc4c(N5CCCCC5COC)nc(OC[C@@]56CCCN5C[C@H](F)C6)nc4c3F)c12 ; C#Cc1c(F)ccc2cc(O)cc(-c3ncc4c(N5CCCCC5COC)nc(OC[C@@]56CCCN5C[C@H](F)C6)nc4c3F)c12] 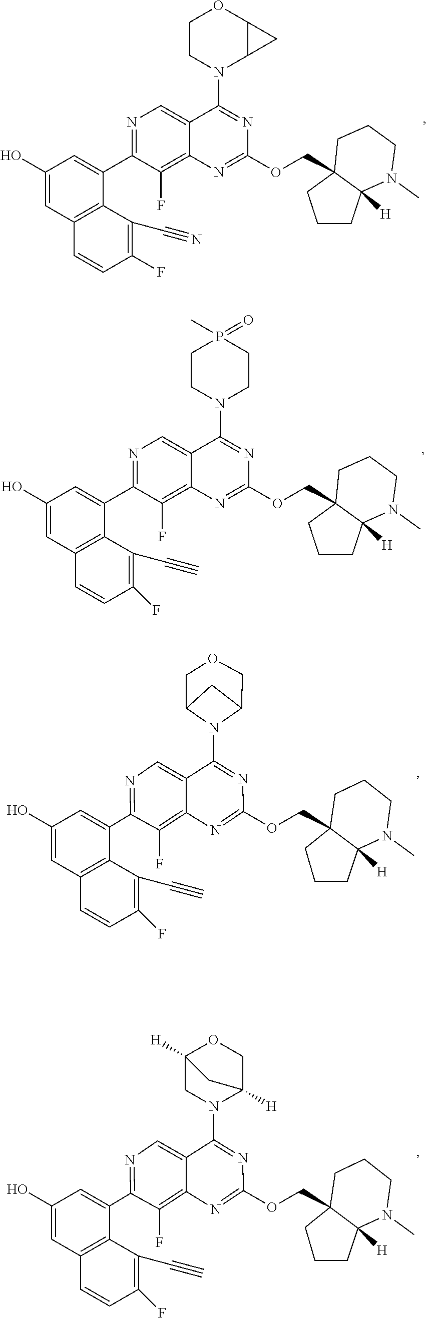 [SMILES: CN1CCC[C@@]2(COc3nc(N4CCOC5CC54)c4cnc(-c5cc(O)cc6ccc(F)c(C#N)c56)c(F)c4n3)CCC[C@@H]12 ; C#Cc1c(F)ccc2cc(O)cc(-c3ncc4c(N5CCP(C)(=O)CC5)nc(OC[C@]56CCC[C@H]5N(C)CCC6)nc4c3F)c12 ; C#Cc1c(F)ccc2cc(O)cc(-c3ncc4c(N5C6COCC5C6)nc(OC[C@]56CCC[C@H]5N(C)CCC6)nc4c3F)c12 ; C#Cc1c(F)ccc2cc(O)cc(-c3ncc4c(N5C[C@@H]6C[C@H]5CO6)nc(OC[C@]56CCC[C@H]5N(C)CCC6)nc4c3F)c12] 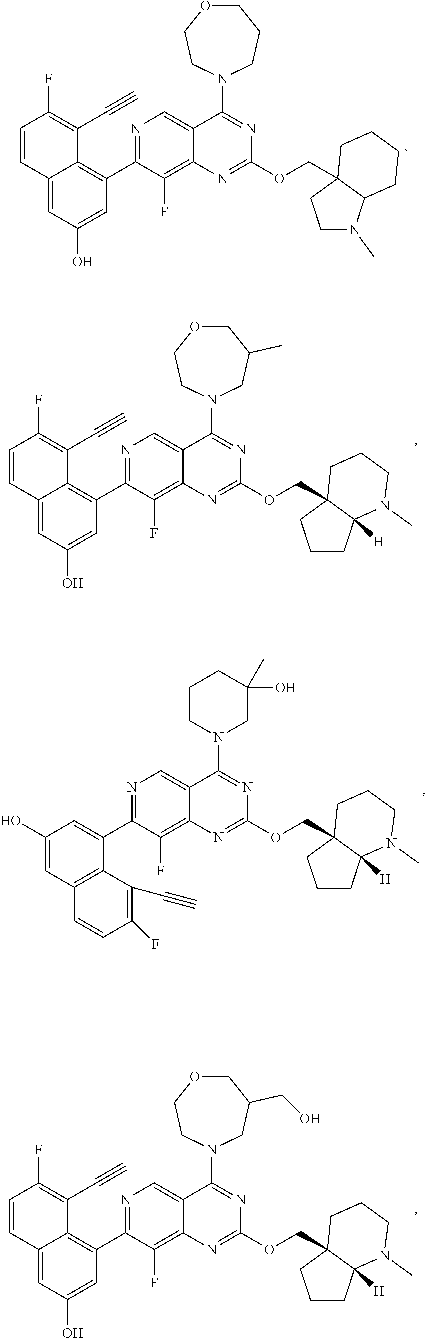 [SMILES: C#Cc1c(F)ccc2cc(O)cc(-c3ncc4c(N5CCCOCC5)nc(OCC56CCCCC5N(C)CC6)nc4c3F)c12 ; C#Cc1c(F)ccc2cc(O)cc(-c3ncc4c(N5CCOCC(C)C5)nc(OC[C@]56CCC[C@H]5N(C)CCC6)nc4c3F)c12 ; C#Cc1c(F)ccc2cc(O)cc(-c3ncc4c(N5CCCC(C)(O)C5)nc(OC[C@]56CCC[C@H]5N(C)CCC6)nc4c3F)c12 ; C#Cc1c(F)ccc2cc(O)cc(-c3ncc4c(N5CCOCC(CO)C5)nc(OC[C@]56CCC[C@H]5N(C)CCC6)nc4c3F)c12] 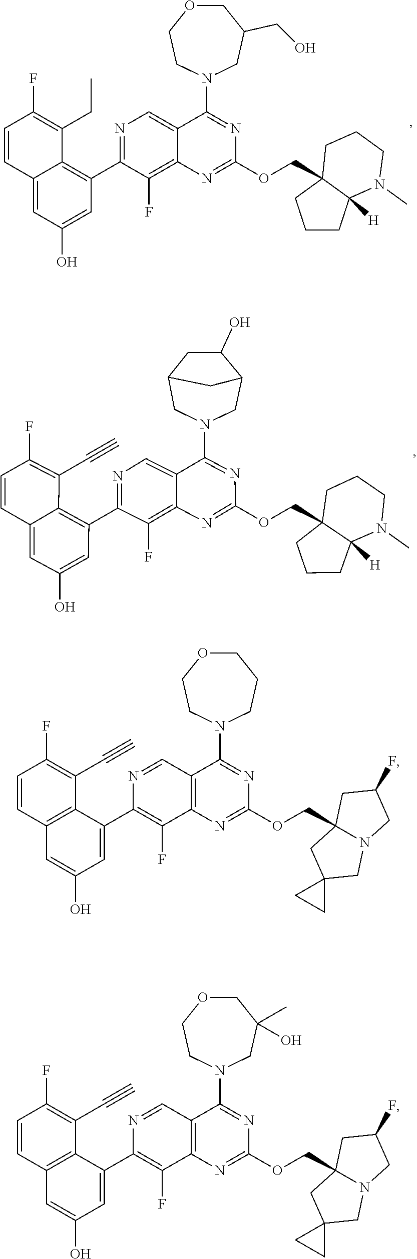 [SMILES: CCc1c(F)ccc2cc(O)cc(-c3ncc4c(N5CCOCC(CO)C5)nc(OC[C@]56CCC[C@H]5N(C)CCC6)nc4c3F)c12 ; C#Cc1c(F)ccc2cc(O)cc(-c3ncc4c(N5CC6CC(O)C(C6)C5)nc(OC[C@]56CCC[C@H]5N(C)CCC6)nc4c3F)c12 ; C#Cc1c(F)ccc2cc(O)cc(-c3ncc4c(N5CCCOCC5)nc(OC[C@@]56C[C@@H](F)CN5CC5(CC5)C6)nc4c3F)c12 ; C#Cc1c(F)ccc2cc(O)cc(-c3ncc4c(N5CCOCC(C)(O)C5)nc(OC[C@@]56C[C@@H](F)CN5CC5(CC5)C6)nc4c3F)c12] 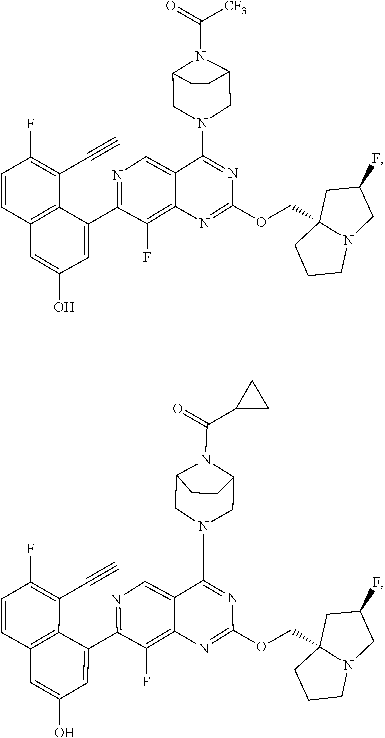 [SMILES: C#Cc1c(F)ccc2cc(O)cc(-c3ncc4c(N5CC6CCC(C5)N6C(=O)C(F)(F)F)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)C5CC5)nc(OC[C@@]56CCCN5C[C@H](F)C6)nc4c3F)c12] 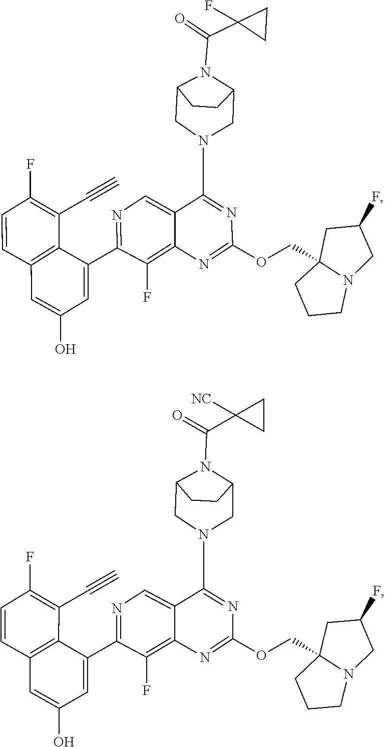 [SMILES: C#Cc1c(F)ccc2cc(O)cc(-c3ncc4c(N5CC6CCC(C5)N6C(=O)C5(F)CC5)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)C5(C#N)CC5)nc(OC[C@@]56CCCN5C[C@H](F)C6)nc4c3F)c12] 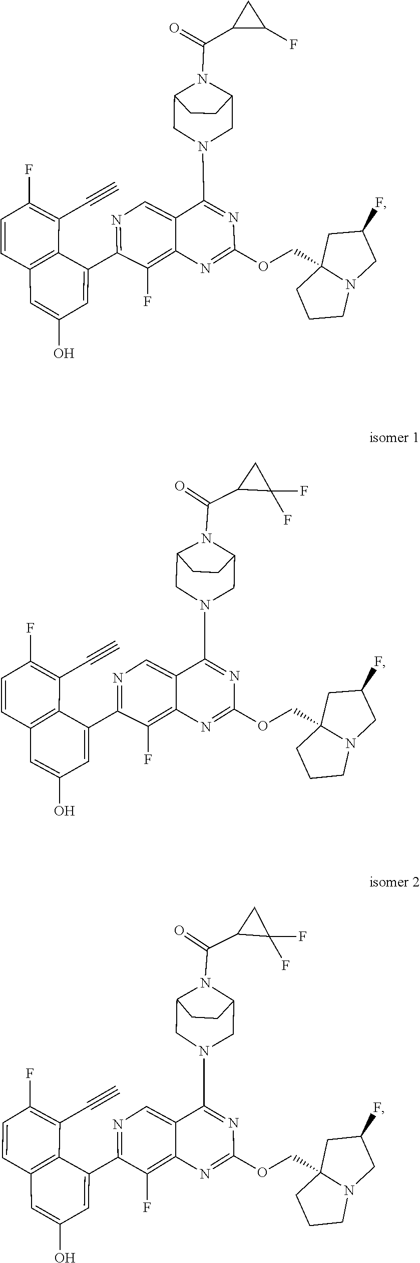 [SMILES: C#Cc1c(F)ccc2cc(O)cc(-c3ncc4c(N5CC6CCC(C5)N6C(=O)C5CC5F)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)C5CC5(F)F)nc(OC[C@@]56CCCN5C[C@H](F)C6)nc4c3F)c12 ; C#Cc1c(F)ccc2cc(O)cc(-c3ncc4c(N5CC6CCC(C5)N6C(=O)C5CC5(F)F)nc(OC[C@@]56CCCN5C[C@H](F)C6)nc4c3F)c12] 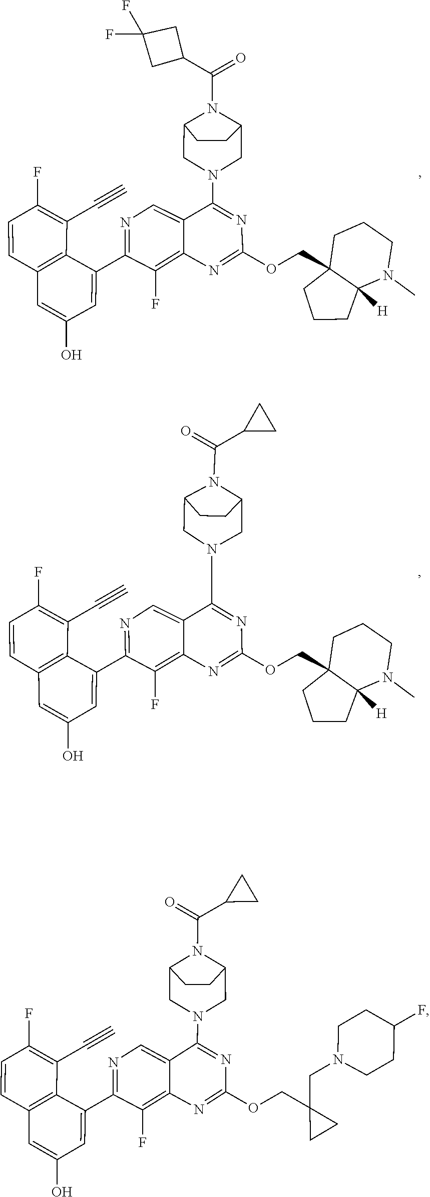 [SMILES: C#Cc1c(F)ccc2cc(O)cc(-c3ncc4c(N5CC6CCC(C5)N6C(=O)C5CC(F)(F)C5)nc(OC[C@]56CCC[C@H]5N(C)CCC6)nc4c3F)c12 ; C#Cc1c(F)ccc2cc(O)cc(-c3ncc4c(N5CC6CCC(C5)N6C(=O)C5CC5)nc(OC[C@]56CCC[C@H]5N(C)CCC6)nc4c3F)c12 ; C#Cc1c(F)ccc2cc(O)cc(-c3ncc4c(N5CC6CCC(C5)N6C(=O)C5CC5)nc(OCC5(CN6CCC(F)CC6)CC5)nc4c3F)c12] 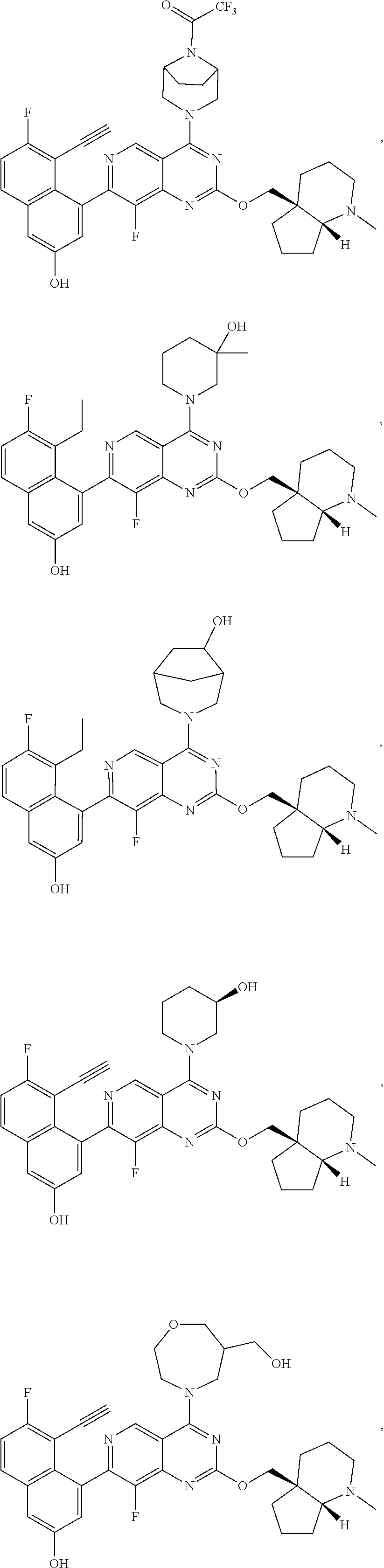 [SMILES: C#Cc1c(F)ccc2cc(O)cc(-c3ncc4c(N5CC6CCC(C5)N6C(=O)C(F)(F)F)nc(OC[C@]56CCC[C@H]5N(C)CCC6)nc4c3F)c12 ; CCc1c(F)ccc2cc(O)cc(-c3ncc4c(N5CCCC(C)(O)C5)nc(OC[C@]56CCC[C@H]5N(C)CCC6)nc4c3F)c12 ; CCc1c(F)ccc2cc(O)cc(-c3ncc4c(N5CC6CC(O)C(C6)C5)nc(OC[C@]56CCC[C@H]5N(C)CCC6)nc4c3F)c12 ; C#Cc1c(F)ccc2cc(O)cc(-c3ncc4c(N5CCC[C@@H](O)C5)nc(OC[C@]56CCC[C@H]5N(C)CCC6)nc4c3F)c12 ; C#Cc1c(F)ccc2cc(O)cc(-c3ncc4c(N5CCOCC(CO)C5)nc(OC[C@]56CCC[C@H]5N(C)CCC6)nc4c3F)c12] 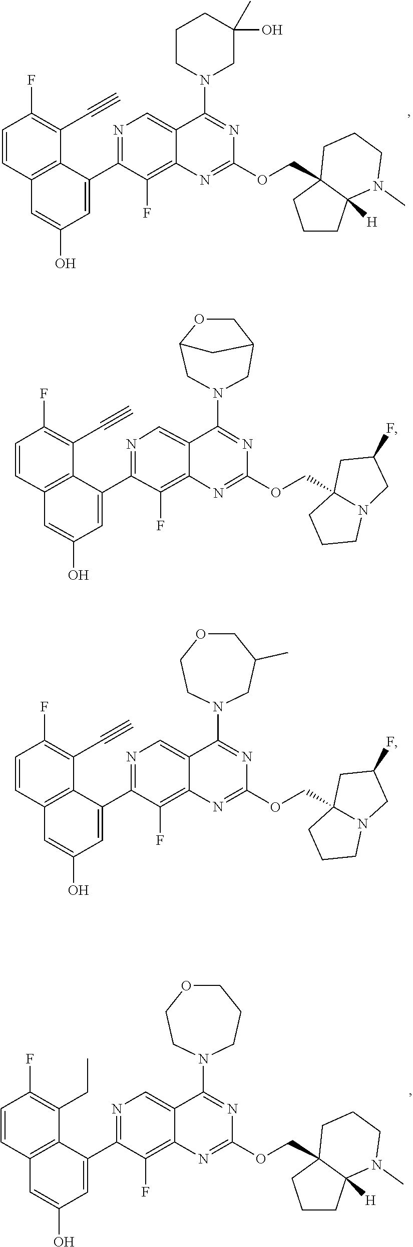 [SMILES: C#Cc1c(F)ccc2cc(O)cc(-c3ncc4c(N5CCCC(C)(O)C5)nc(OC[C@]56CCC[C@H]5N(C)CCC6)nc4c3F)c12 ; C#Cc1c(F)ccc2cc(O)cc(-c3ncc4c(N5CC6COC(C6)C5)nc(OC[C@@]56CCCN5C[C@H](F)C6)nc4c3F)c12 ; C#Cc1c(F)ccc2cc(O)cc(-c3ncc4c(N5CCOCC(C)C5)nc(OC[C@@]56CCCN5C[C@H](F)C6)nc4c3F)c12 ; CCc1c(F)ccc2cc(O)cc(-c3ncc4c(N5CCCOCC5)nc(OC[C@]56CCC[C@H]5N(C)CCC6)nc4c3F)c12] 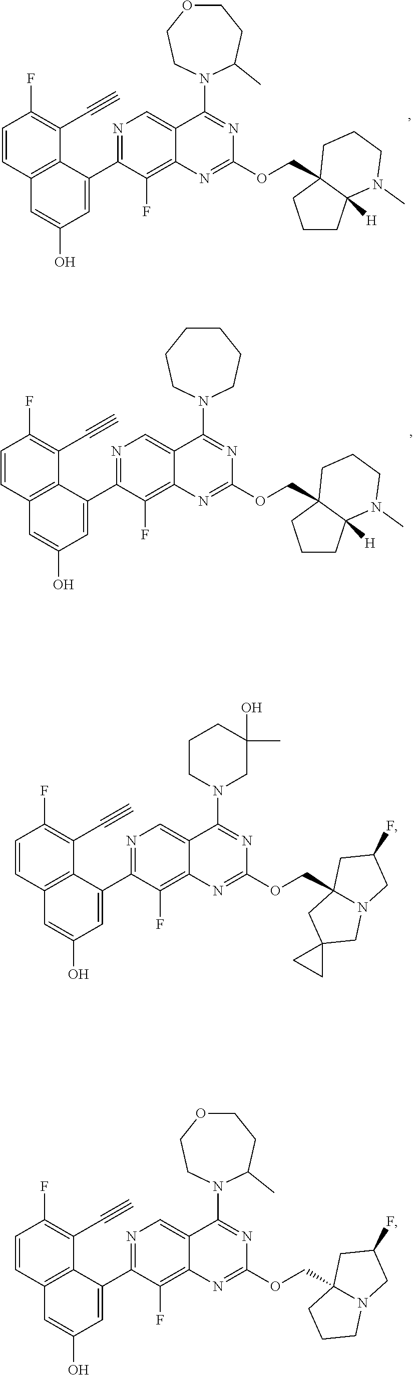 [SMILES: C#Cc1c(F)ccc2cc(O)cc(-c3ncc4c(N5CCOCCC5C)nc(OC[C@]56CCC[C@H]5N(C)CCC6)nc4c3F)c12 ; C#Cc1c(F)ccc2cc(O)cc(-c3ncc4c(N5CCCCCC5)nc(OC[C@]56CCC[C@H]5N(C)CCC6)nc4c3F)c12 ; C#Cc1c(F)ccc2cc(O)cc(-c3ncc4c(N5CCCC(C)(O)C5)nc(OC[C@@]56C[C@@H](F)CN5CC5(CC5)C6)nc4c3F)c12 ; C#Cc1c(F)ccc2cc(O)cc(-c3ncc4c(N5CCOCCC5C)nc(OC[C@@]56CCCN5C[C@H](F)C6)nc4c3F)c12] 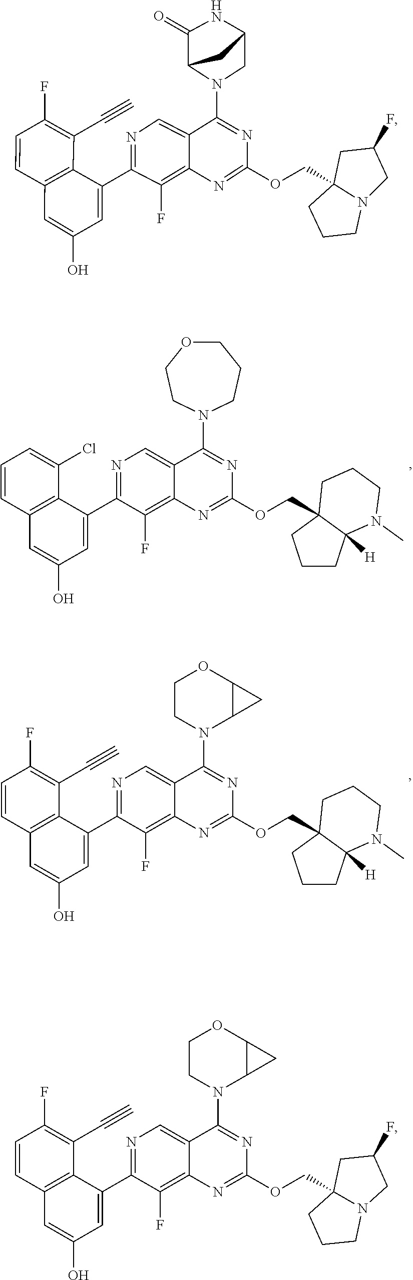 [SMILES: C#Cc1c(F)ccc2cc(O)cc(-c3ncc4c(N5C[C@@H]6C[C@H]5C(=O)N6)nc(OC[C@@]56CCCN5C[C@H](F)C6)nc4c3F)c12 ; CN1CCC[C@@]2(COc3nc(N4CCCOCC4)c4cnc(-c5cc(O)cc6cccc(Cl)c56)c(F)c4n3)CCC[C@@H]12 ; C#Cc1c(F)ccc2cc(O)cc(-c3ncc4c(N5CCOC6CC65)nc(OC[C@]56CCC[C@H]5N(C)CCC6)nc4c3F)c12 ; C#Cc1c(F)ccc2cc(O)cc(-c3ncc4c(N5CCOC6CC65)nc(OC[C@@]56CCCN5C[C@H](F)C6)nc4c3F)c12] 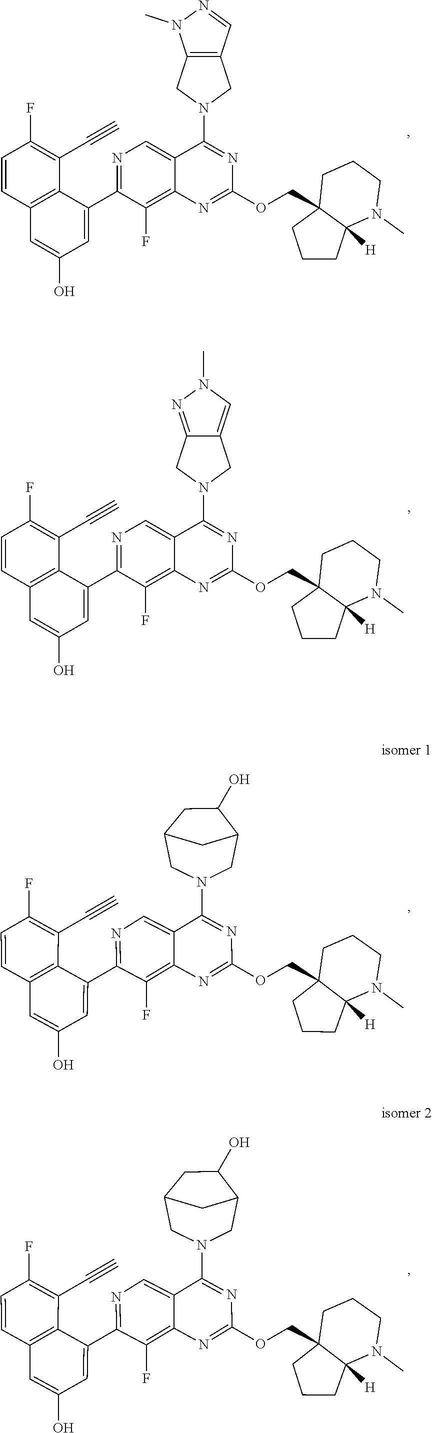 [SMILES: C#Cc1c(F)ccc2cc(O)cc(-c3ncc4c(N5Cc6cnn(C)c6C5)nc(OC[C@]56CCC[C@H]5N(C)CCC6)nc4c3F)c12 ; C#Cc1c(F)ccc2cc(O)cc(-c3ncc4c(N5Cc6cn(C)nc6C5)nc(OC[C@]56CCC[C@H]5N(C)CCC6)nc4c3F)c12 ; C#Cc1c(F)ccc2cc(O)cc(-c3ncc4c(N5CC6CC(O)C(C6)C5)nc(OC[C@]56CCC[C@H]5N(C)CCC6)nc4c3F)c12 ; C#Cc1c(F)ccc2cc(O)cc(-c3ncc4c(N5CC6CC(O)C(C6)C5)nc(OC[C@]56CCC[C@H]5N(C)CCC6)nc4c3F)c12] 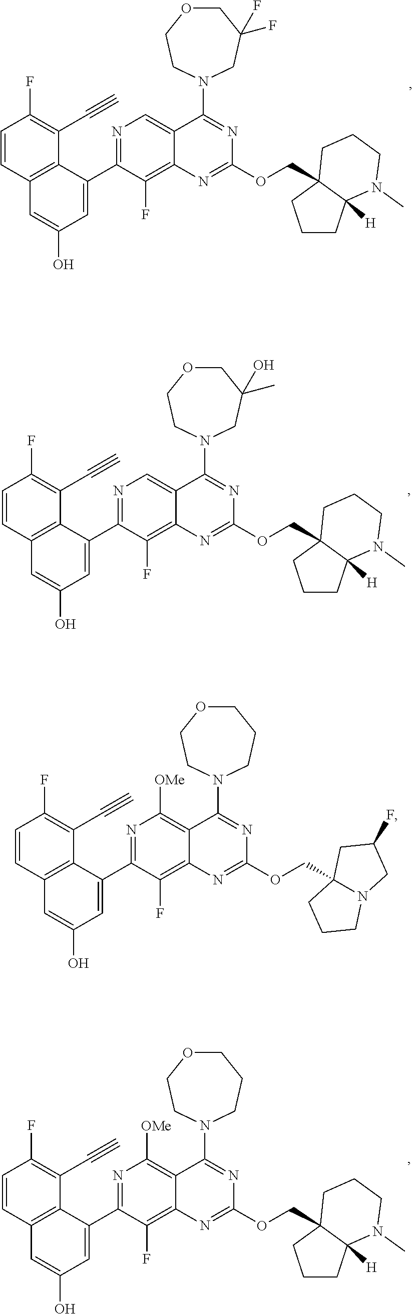 [SMILES: C#Cc1c(F)ccc2cc(O)cc(-c3ncc4c(N5CCOCC(F)(F)C5)nc(OC[C@]56CCC[C@H]5N(C)CCC6)nc4c3F)c12 ; C#Cc1c(F)ccc2cc(O)cc(-c3ncc4c(N5CCOCC(C)(O)C5)nc(OC[C@]56CCC[C@H]5N(C)CCC6)nc4c3F)c12 ; C#Cc1c(F)ccc2cc(O)cc(-c3nc(OC)c4c(N5CCCOCC5)nc(OC[C@@]56CCCN5C[C@H](F)C6)nc4c3F)c12 ; C#Cc1c(F)ccc2cc(O)cc(-c3nc(OC)c4c(N5CCCOCC5)nc(OC[C@]56CCC[C@H]5N(C)CCC6)nc4c3F)c12] 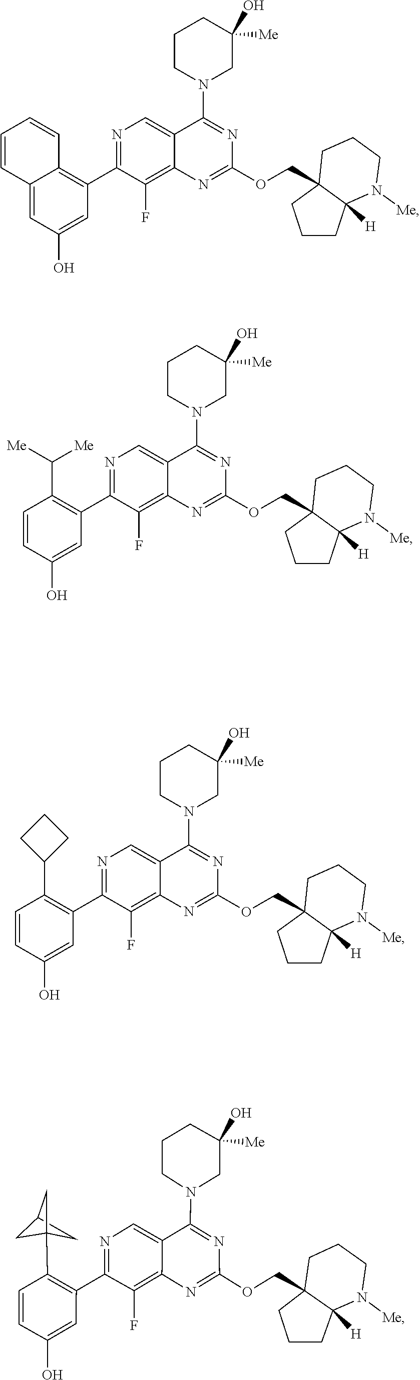 [SMILES: CN1CCC[C@@]2(COc3nc(N4CCC[C@@](C)(O)C4)c4cnc(-c5cc(O)cc6ccccc56)c(F)c4n3)CCC[C@@H]12 ; CC(C)c1ccc(O)cc1-c1ncc2c(N3CCC[C@@](C)(O)C3)nc(OC[C@]34CCC[C@H]3N(C)CCC4)nc2c1F ; CN1CCC[C@@]2(COc3nc(N4CCC[C@@](C)(O)C4)c4cnc(-c5cc(O)ccc5C5CCC5)c(F)c4n3)CCC[C@@H]12 ; CN1CCC[C@@]2(COc3nc(N4CCC[C@@](C)(O)C4)c4cnc(-c5cc(O)ccc5C56CC(C5)C6)c(F)c4n3)CCC[C@@H]12] 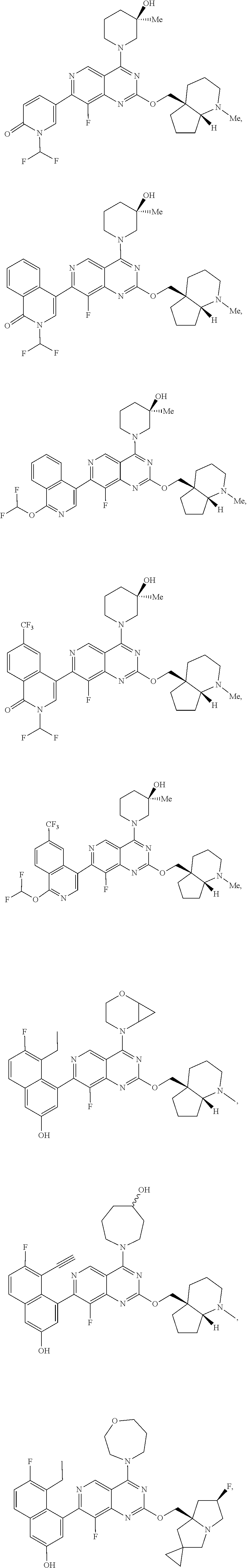 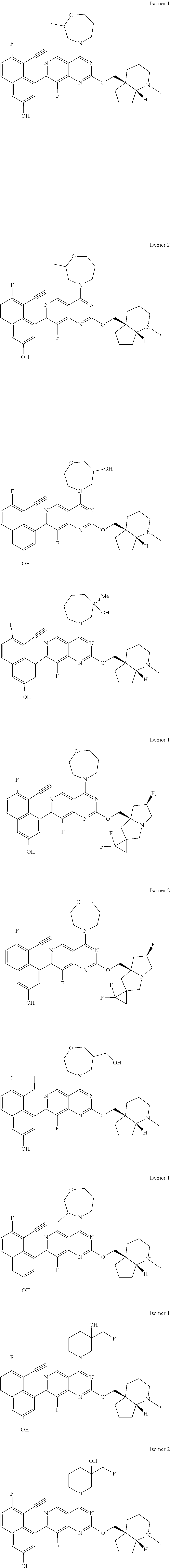 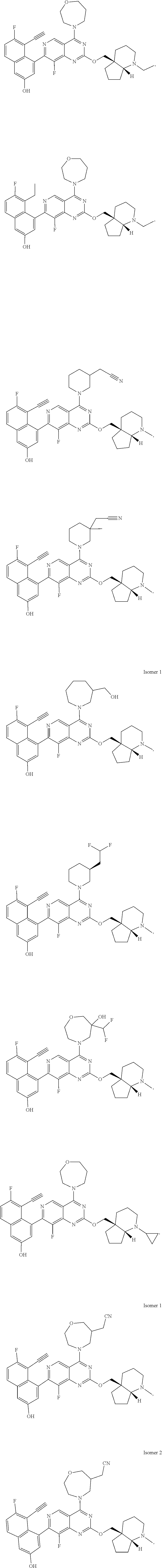 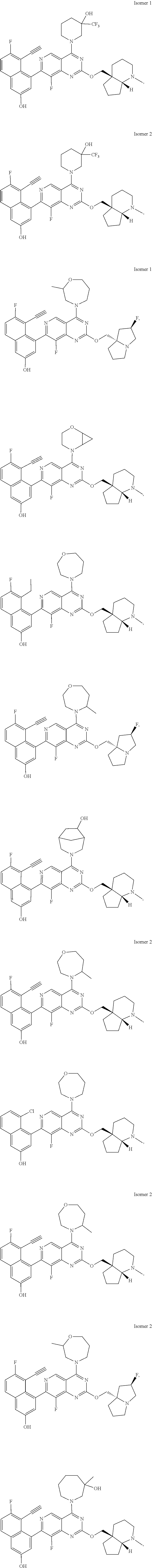 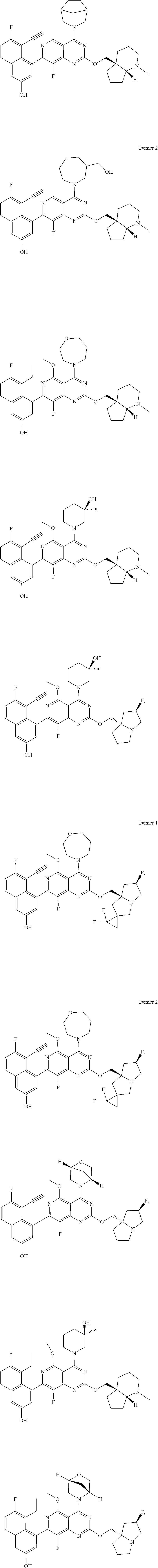 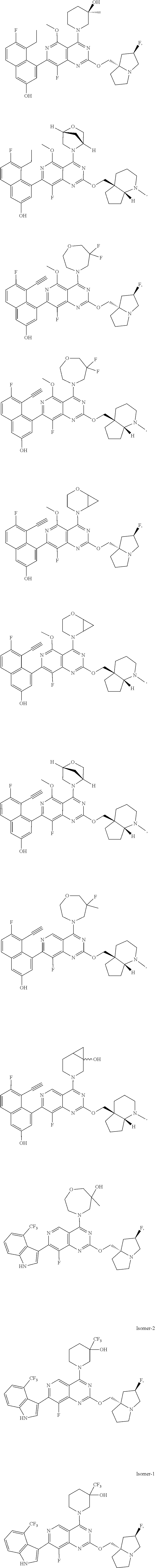 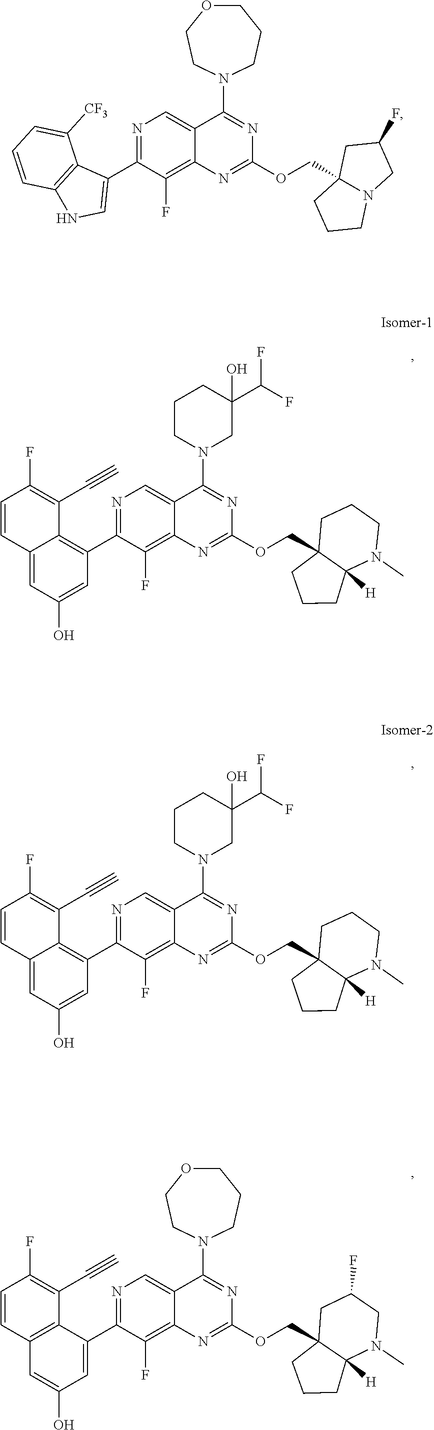 [SMILES: Fc1c(-c2c[nH]c3cccc(C(F)(F)F)c23)ncc2c(N3CCCOCC3)nc(OC[C@@]34CCCN3C[C@H](F)C4)nc12 ; C#Cc1c(F)ccc2cc(O)cc(-c3ncc4c(N5CCCC(O)(C(F)F)C5)nc(OC[C@]56CCC[C@H]5N(C)CCC6)nc4c3F)c12 ; C#Cc1c(F)ccc2cc(O)cc(-c3ncc4c(N5CCCC(O)(C(F)F)C5)nc(OC[C@]56CCC[C@H]5N(C)CCC6)nc4c3F)c12 ; C#Cc1c(F)ccc2cc(O)cc(-c3ncc4c(N5CCCOCC5)nc(OC[C@]56CCC[C@H]5N(C)C[C@@H](F)C6)nc4c3F)c12] 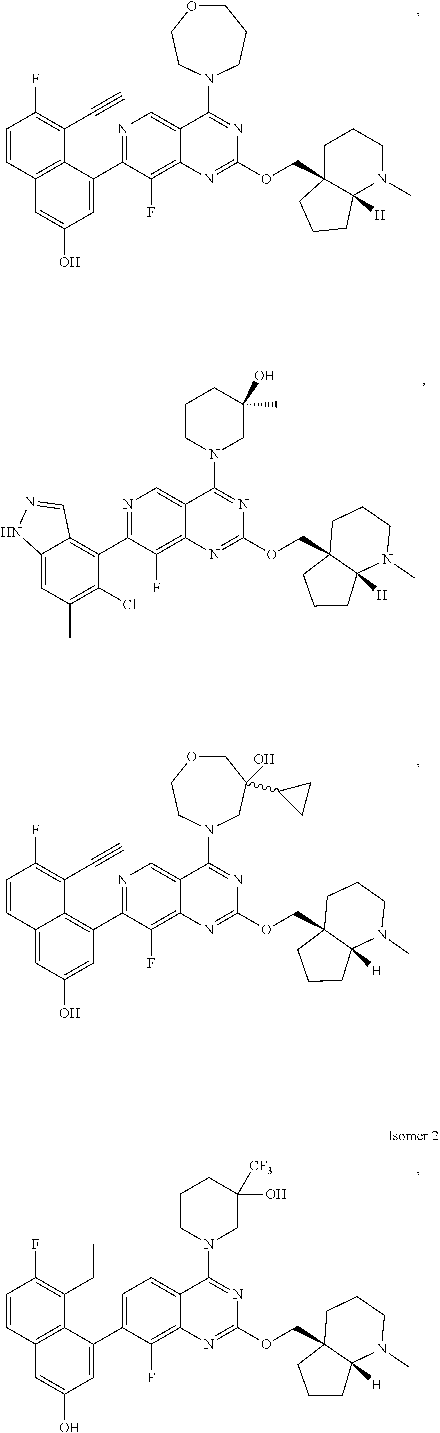 [SMILES: C#Cc1c(F)ccc2cc(O)cc(-c3ncc4c(N5CCCOCC5)nc(OC[C@]56CCC[C@H]5N(C)CCC6)nc4c3F)c12 ; Cc1cc2[nH]ncc2c(-c2ncc3c(N4CCC[C@@](C)(O)C4)nc(OC[C@]45CCC[C@H]4N(C)CCC5)nc3c2F)c1Cl ; C#Cc1c(F)ccc2cc(O)cc(-c3ncc4c(N5CCOCC(O)(C6CC6)C5)nc(OC[C@]56CCC[C@H]5N(C)CCC6)nc4c3F)c12 ; CCc1c(F)ccc2cc(O)cc(-c3ccc4c(N5CCCC(O)(C(F)(F)F)C5)nc(OC[C@]56CCC[C@H]5N(C)CCC6)nc4c3F)c12] 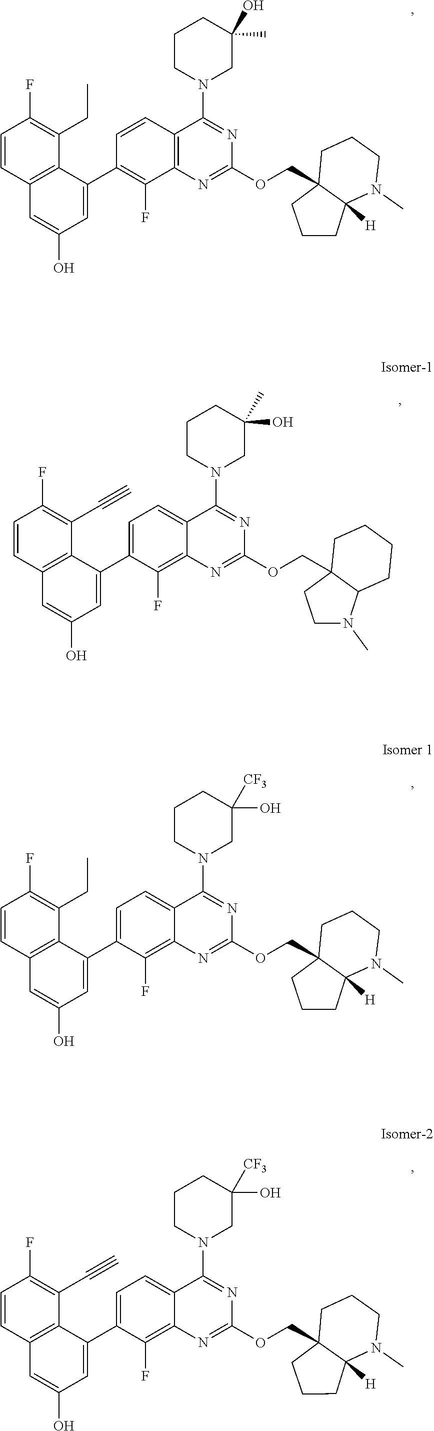 [SMILES: CCc1c(F)ccc2cc(O)cc(-c3ccc4c(N5CCC[C@@](C)(O)C5)nc(OC[C@]56CCC[C@H]5N(C)CCC6)nc4c3F)c12 ; C#Cc1c(F)ccc2cc(O)cc(-c3ccc4c(N5CCC[C@@](C)(O)C5)nc(OCC56CCCCC5N(C)CC6)nc4c3F)c12 ; CCc1c(F)ccc2cc(O)cc(-c3ccc4c(N5CCCC(O)(C(F)(F)F)C5)nc(OC[C@]56CCC[C@H]5N(C)CCC6)nc4c3F)c12 ; C#Cc1c(F)ccc2cc(O)cc(-c3ccc4c(N5CCCC(O)(C(F)(F)F)C5)nc(OC[C@]56CCC[C@H]5N(C)CCC6)nc4c3F)c12] 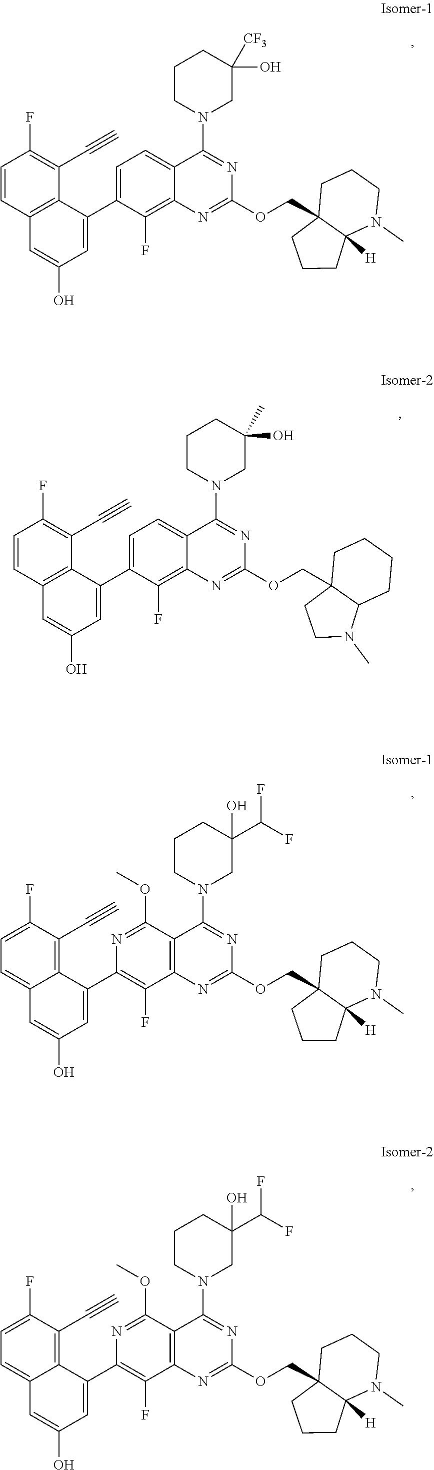 [SMILES: C#Cc1c(F)ccc2cc(O)cc(-c3ccc4c(N5CCCC(O)(C(F)(F)F)C5)nc(OC[C@]56CCC[C@H]5N(C)CCC6)nc4c3F)c12 ; C#Cc1c(F)ccc2cc(O)cc(-c3ccc4c(N5CCC[C@@](C)(O)C5)nc(OCC56CCCCC5N(C)CC6)nc4c3F)c12 ; C#Cc1c(F)ccc2cc(O)cc(-c3nc(OC)c4c(N5CCCC(O)(C(F)F)C5)nc(OC[C@]56CCC[C@H]5N(C)CCC6)nc4c3F)c12 ; C#Cc1c(F)ccc2cc(O)cc(-c3nc(OC)c4c(N5CCCC(O)(C(F)F)C5)nc(OC[C@]56CCC[C@H]5N(C)CCC6)nc4c3F)c12] 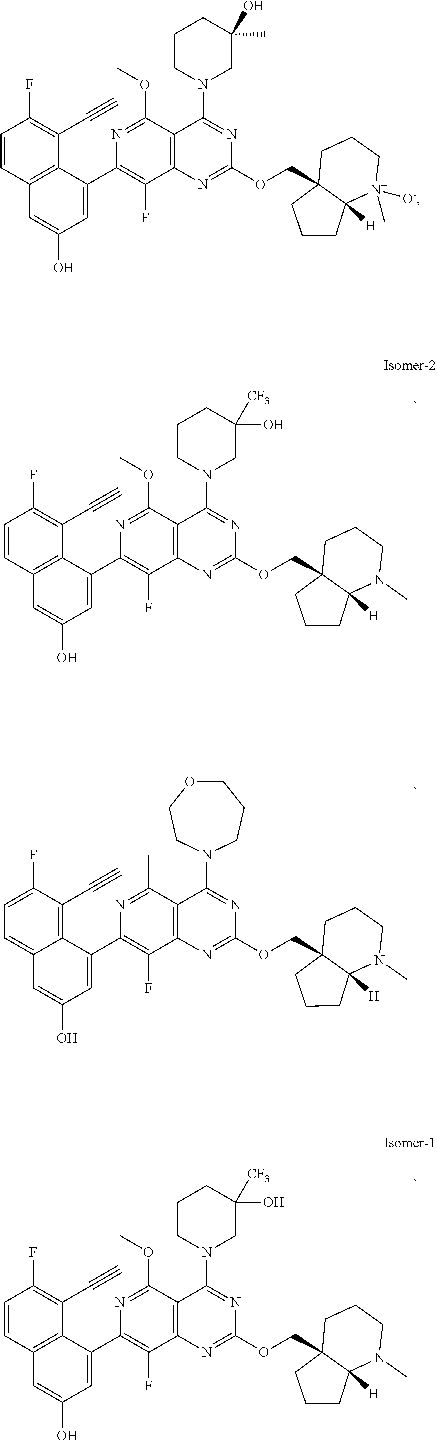 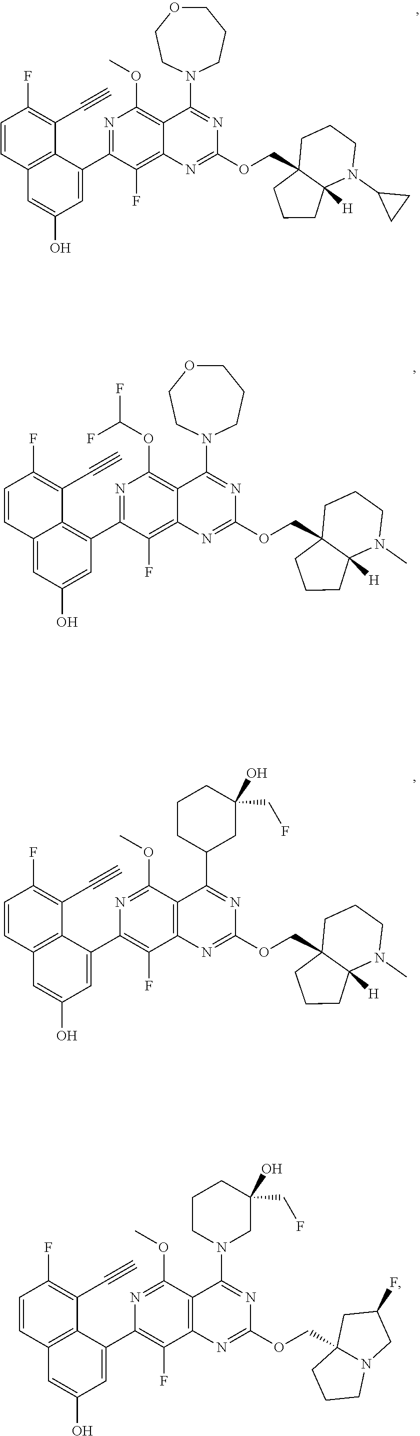 [SMILES: C#Cc1c(F)ccc2cc(O)cc(-c3nc(OC)c4c(N5CCCOCC5)nc(OC[C@]56CCC[C@H]5N(C5CC5)CCC6)nc4c3F)c12 ; C#Cc1c(F)ccc2cc(O)cc(-c3nc(OC(F)F)c4c(N5CCCOCC5)nc(OC[C@]56CCC[C@H]5N(C)CCC6)nc4c3F)c12 ; C#Cc1c(F)ccc2cc(O)cc(-c3nc(OC)c4c(C5CCC[C@](O)(CF)C5)nc(OC[C@]56CCC[C@H]5N(C)CCC6)nc4c3F)c12 ; C#Cc1c(F)ccc2cc(O)cc(-c3nc(OC)c4c(N5CCC[C@](O)(CF)C5)nc(OC[C@@]56CCCN5C[C@H](F)C6)nc4c3F)c12] 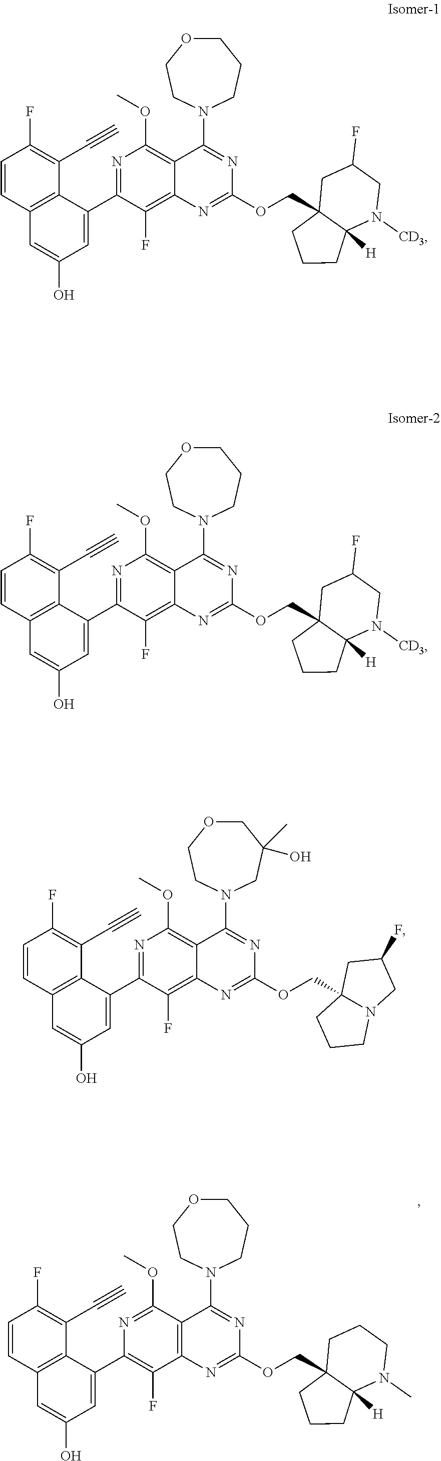 [SMILES: [2H]C([2H])([2H])N1CC(F)C[C@@]2(COc3nc(N4CCCOCC4)c4c(OC)nc(-c5cc(O)cc6ccc(F)c(C#C)c56)c(F)c4n3)CCC[C@@H]12 ; [2H]C([2H])([2H])N1CC(F)C[C@@]2(COc3nc(N4CCCOCC4)c4c(OC)nc(-c5cc(O)cc6ccc(F)c(C#C)c56)c(F)c4n3)CCC[C@@H]12 ; C#Cc1c(F)ccc2cc(O)cc(-c3nc(OC)c4c(N5CCOCC(C)(O)C5)nc(OC[C@@]56CCCN5C[C@H](F)C6)nc4c3F)c12 ; C#Cc1c(F)ccc2cc(O)cc(-c3nc(OC)c4c(N5CCCOCC5)nc(OC[C@]56CCC[C@H]5N(C)CCC6)nc4c3F)c12] 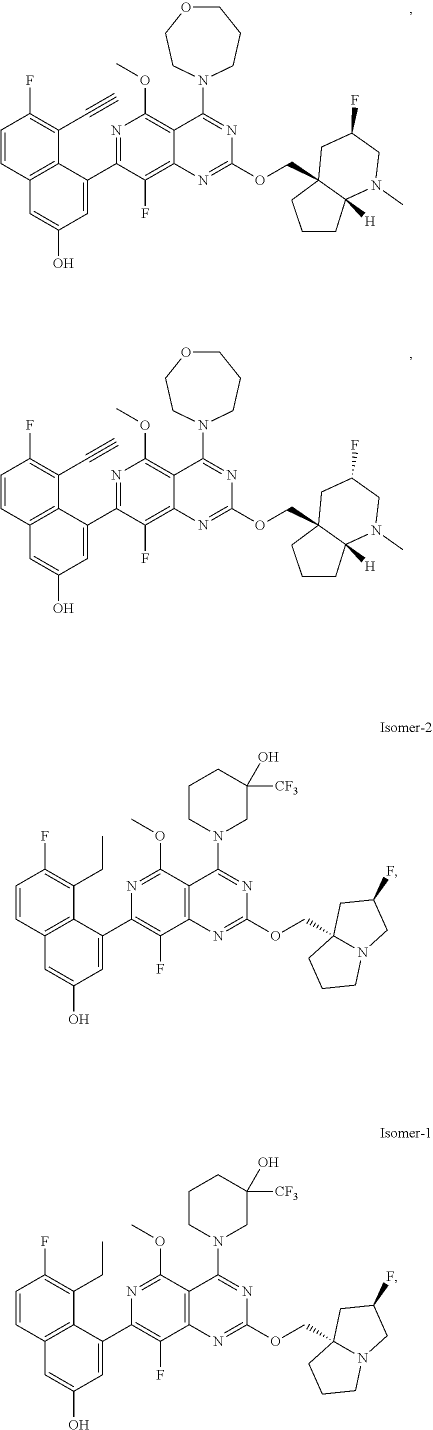 [SMILES: C#Cc1c(F)ccc2cc(O)cc(-c3nc(OC)c4c(N5CCCOCC5)nc(OC[C@]56CCC[C@H]5N(C)C[C@H](F)C6)nc4c3F)c12 ; C#Cc1c(F)ccc2cc(O)cc(-c3nc(OC)c4c(N5CCCOCC5)nc(OC[C@]56CCC[C@H]5N(C)C[C@@H](F)C6)nc4c3F)c12 ; CCc1c(F)ccc2cc(O)cc(-c3nc(OC)c4c(N5CCCC(O)(C(F)(F)F)C5)nc(OC[C@@]56CCCN5C[C@H](F)C6)nc4c3F)c12 ; CCc1c(F)ccc2cc(O)cc(-c3nc(OC)c4c(N5CCCC(O)(C(F)(F)F)C5)nc(OC[C@@]56CCCN5C[C@H](F)C6)nc4c3F)c12] 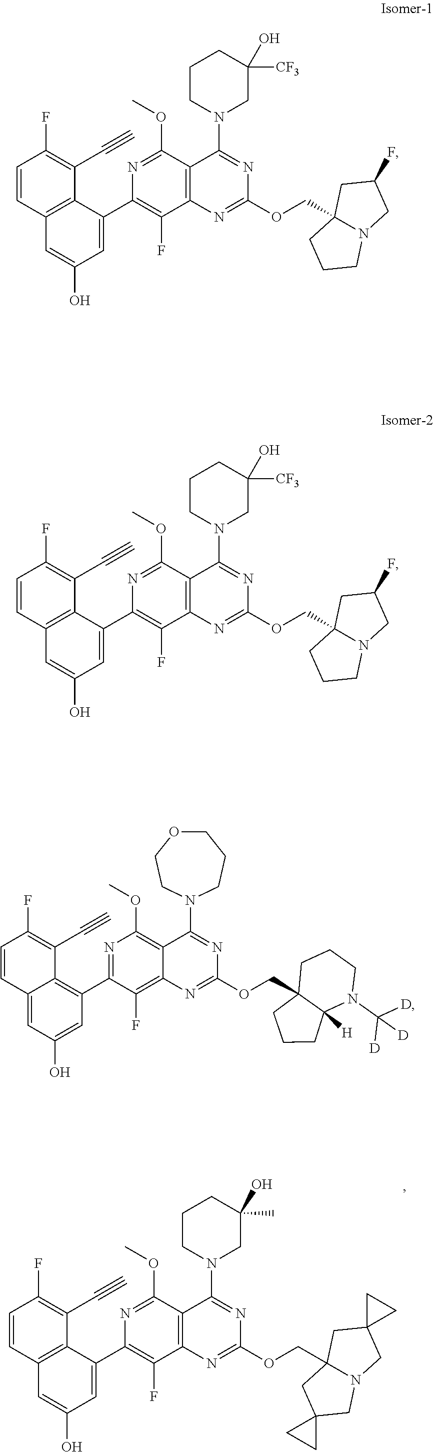 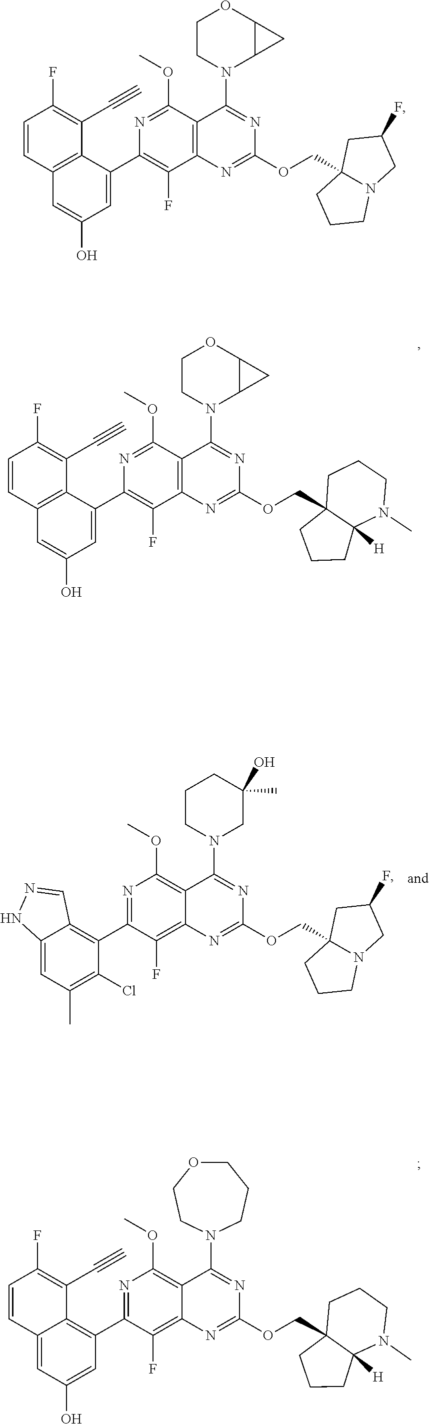 [SMILES: C#Cc1c(F)ccc2cc(O)cc(-c3nc(OC)c4c(N5CCOC6CC65)nc(OC[C@@]56CCCN5C[C@H](F)C6)nc4c3F)c12 ; C#Cc1c(F)ccc2cc(O)cc(-c3nc(OC)c4c(N5CCOC6CC65)nc(OC[C@]56CCC[C@H]5N(C)CCC6)nc4c3F)c12 ; COc1nc(-c2c(Cl)c(C)cc3[nH]ncc23)c(F)c2nc(OC[C@@]34CCCN3C[C@H](F)C4)nc(N3CCC[C@@](C)(O)C3)c12 ; C#Cc1c(F)ccc2cc(O)cc(-c3nc(OC)c4c(N5CCCOCC5)nc(OC[C@]56CCC[C@H]5N(C)CCC6)nc4c3F)c12]
or a pharmaceutically acceptable salt thereof.

44. (canceled)

45. A pharmaceutical composition comprising a compound of claim 4, or a pharmaceutically acceptable salt thereof, and one or more pharmaceutically acceptable excipients.

46. An oral dosage form comprising a compound of claim 4, or a pharmaceutically acceptable salt thereof, and one or more pharmaceutically acceptable excipients.

47. A method of inhibiting KRAS G12A, KRAS G12C, KRAS G12D, KRAS G12R, KRAS G12S, KRAS G12V, KRAS G13D and/or KRAS Q61H in a subject in need thereof, the method comprising administering to the subject a compound, composition, or dosage form of claim 4, or a pharmaceutically acceptable salt thereof.

48. A method for treating a cancer susceptible to KRAS G12A, KRAS G12C, KRAS G12D, KRAS G12R, KRAS G12S, KRAS G12V, KRAS G13D and/or KRAS Q61H inhibition in a subject in need thereof, the method comprising administering to the subject a compound, composition, or dosage form of claim 4, or a pharmaceutically acceptable salt thereof.

49. A method for treating a cancer expressing KRAS G12A, KRAS G12C, KRAS G12D, KRAS G12R, KRAS G12S, KRAS G12V, KRAS G13D and/or KRAS Q61H mutation in a subject in need thereof, the method comprising administering to the subject a compound, composition, or dosage form of claim 4, or a pharmaceutically acceptable salt thereof.

50. A method for treating a cancer in a subject in need thereof, the method comprising administering to the subject a compound, composition, or dosage form of claim 4, or a pharmaceutically acceptable salt thereof.

51. The method of claim 50, wherein the cancer is pancreatic cancer, colorectal cancer, lung cancer, gastric cancer, breast cancer, bladder cancer, cervical cancer, ovarian cancer, cancer of the uterus, or a combination thereof.

52. The method of claim 51, wherein the cancer is non-small cell lung cancer.
